SEQUENCE LISTING (70 sequences)

SEQ ID NO: 1 - protein, 6 aa, Artificial Sequence
  Synthetic protein-like multiblock polymer hexapeptide
  MISC_FEATURE (6)..(6): X can be alanine, tyrosine, valine, or serine
GAGAGX

SEQ ID NO: 2 - protein, 6 aa, Artificial Sequence
  Synthetic protein-like multiblock polymer hexapeptide
GAGAGS

SEQ ID NO: 3 - protein, 5 aa, Artificial Sequence
  Synthetic mammalian elastin
VPGVG

SEQ ID NO: 4 - protein, 5 aa, Artificial Sequence
  Synthetic mammalian elastin
GVGVP

SEQ ID NO: 5 - protein, 44 aa, Artificial Sequence
  Synthetic protein.
SGFGPVANGGSGEASSESDFGSSGFGPVANASSGEASSESDFAG

SEQ ID NO: 6 - protein, 5 aa, Artificial Sequence
  Synthetic consensus peptide
  MISC_FEATURE (4)..(5): X can be alanine, serine, glycine, tyrosine, proline, or glutamine
GPGXX

SEQ ID NO: 7 - protein, 9 aa, Artificial Sequence
  Synthetic pro-resilin elastomeric protein
GGRPSDTYG

SEQ ID NO: 8 - protein, 9 aa, Artificial Sequence
  Synthetic pro-resilin elastomeric protein
GGRPSSSYG

SEQ ID NO: 9 - protein, 35 aa, Artificial Sequence
  Derived from Araneus diadematus
GSSAAAAAAAASGPGGYGPENQGPSGPGGYGPGGP

SEQ ID NO: 10 - protein, 5 aa, Artificial Sequence
  Synthetic recombinant silk protein repetitive unit
GPGAS

SEQ ID NO: 11 - protein, 5 aa, Artificial Sequence
  Synthetic recombinant silk protein repetitive unit
GPGSG

SEQ ID NO: 12 - protein, 5 aa, Artificial Sequence
  Synthetic recombinant silk protein repetitive unit
GPGGY

SEQ ID NO: 13 - protein, 5 aa, Artificial Sequence
  Synthetic recombinant silk protein repetitive unit
GPGGP

SEQ ID NO: 14 - protein, 5 aa, Artificial Sequence
  Synthetic recombinant silk protein repetitive unit
GPGGA

SEQ ID NO: 15 - protein, 5 aa, Artificial Sequence
  Synthetic recombinant silk protein repetitive unit
GPGQQ

SEQ ID NO: 16 - protein, 5 aa, Artificial Sequence
  Synthetic recombinant silk protein repetitive unit
GPGGG

SEQ ID NO: 17 - protein, 5 aa, Artificial Sequence
  Synthetic recombinant silk protein repetitive unit
GPGQG

SEQ ID NO: 18 - protein, 5 aa, Artificial Sequence
  Synthetic recombinant silk protein repetitive unit
GPGGS

SEQ ID NO: 19 - protein, 5 aa, Artificial Sequence
  Synthetic recombinant silk protein repetitive unit
AAAAA

SEQ ID NO: 20 - protein, 6 aa, Artificial Sequence
  Synthetic recombinant silk protein repetitive unit
AAAAAA

SEQ ID NO: 21 - protein, 7 aa, Artificial Sequence
  Synthetic recombinant silk protein repetitive unit
AAAAAAA

SEQ ID NO: 22 - protein, 8 aa, Artificial Sequence
  Synthetic recombinant silk protein repetitive unit
AAAAAAAA

SEQ ID NO: 23 - protein, 9 aa, Artificial Sequence
  Synthetic recombinant silk protein repetitive unit
AAAAAAAAA

SEQ ID NO: 24 - protein, 10 aa, Artificial Sequence
  Synthetic recombinant silk protein repetitive unit
AAAAAAAAAA

SEQ ID NO: 25 - protein, 24 aa, Artificial Sequence
  Synthetic recombinant silk protein repetitive unit
GPYGPGASAAAAAAGGYGPGSGQQ

SEQ ID NO: 26 - protein, 20 aa, Artificial Sequence
  Synthetic recombinant silk protein repetitive unit
GPGQQGPGQQGPGQQGPGQQ

SEQ ID NO: 27 - protein, 27 aa, Artificial Sequence
  Synthetic recombinant silk protein repetitive unit
GPGGAGGPYGPGGAGGPYGPGGAGGPY

SEQ ID NO: 28 - protein, 28 aa, Artificial Sequence
  Synthetic recombinant silk protein repetitive unit
GGTTIIEDLDITIDGADGPITISEELTI

SEQ ID NO: 29 - protein, 34 aa, Artificial Sequence
  Synthetic recombinant silk protein repetitive unit
PGSSAAAAAAAASGPGQGQGQGQGQGGRPSDTYG

SEQ ID NO: 30 - protein, 39 aa, Artificial Sequence
  Synthetic recombinant silk protein repetitive unit
SAAAAAAAAGPGGGNGGRPSDTYGAPGGGNGGRPSSSYG

SEQ ID NO: 31 - protein, 18 aa, Artificial Sequence
  Synthetic recombinant silk protein repetitive unit
GGAGGAGGAGGSGGAGGS

SEQ ID NO: 32 - protein, 30 aa, Artificial Sequence
  Synthetic recombinant silk protein repetitive unit
GPGGAGPGGYGPGGSGPGGYGPGGSGPGGY

SEQ ID NO: 33 - protein, 24 aa, Artificial Sequence
  Synthetic recombinant silk protein repetitive unit
GPYGPGASAAAAAAGGYGPGCGQQ

SEQ ID NO: 34 - protein, 24 aa, Artificial Sequence
  Synthetic recombinant silk protein repetitive unit
GPYGPGASAAAAAAGGYGPGKGQQ

SEQ ID NO: 35 - protein, 35 aa, Artificial Sequence
  Synthetic recombinant silk protein repetitive unit
GSSAAAAAAAASGPGGYGPENQGPCGPGGYGPGGP

SEQ ID NO: 36 - protein, 35 aa, Artificial Sequence
  Synthetic recombinant silk protein repetitive unit
GSSAAAAAAAASGPGGYGPKNQGPSGPGGYGPGGP

SEQ ID NO: 37 - protein, 35 aa, Artificial Sequence
  Synthetic recombinant silk protein repetitive unit
GSSAAAAAAAASGPGGYGPKNQGPCGPGGYGPGGP

SEQ ID NO: 38 - protein, 46 aa, Artificial Sequence
  Synthetic silk protein-like multiblock peptide
  MISC_FEATURE (1)..(20): X can be tyrosine or glutamine
  MISC_FEATURE (7)..(9): XGG may be present or absent
  MISC_FEATURE (16)..(21): each group of GXG may be present or absent
  MISC_FEATURE (37)..(42): each group of AGA may be present or absent
  MISC_FEATURE (44)..(44): G may be present or absent
XGGXGGXGGXGAGXGGXGGXGAGAAGAAGAAGAAGAAGAAGAGGAG

SEQ ID NO: 39 - protein, 43 aa, Artificial Sequence
  Synthetic silk protein-like multiblock peptide
  MISC_FEATURE (21)..(30): GPGGYGPGQQ may be present or absent
  MISC_FEATURE (31)..(32): X can be amino acid sequence GPS or GPG
  MISC_FEATURE (41)..(43): each A may be present or absent
GPGGYGPGQQGPGGYGPGQQGPGGYGPGQQXXSAAAAAAAAAA

SEQ ID NO: 40 - protein, 46 aa, Artificial Sequence
  Synthetic silk protein-like multiblock peptide
  MISC_FEATURE (5)..(14): each sequence of GA may be present or absent
  MISC_FEATURE (15)..(18): each A may be present or absent
  MISC_FEATURE (21)..(30): X can be tyrosine, glutamine, or alanine
  MISC_FEATURE (21)..(30): each sequence of GGX may be present or absent
  MISC_FEATURE (33)..(42): each sequence of GA may be present or absent
  MISC_FEATURE (43)..(46): each A may be present or absent
GRGAGAGAGAGAGAAAAAGGXGGXGGXGGXGAGAGAGAGAGAAAAA

SEQ ID NO: 41 - protein, 26 aa, Artificial Sequence
  Synthetic silk protein-like multiblock peptide
  MISC_FEATURE (3)..(12): X can be tyrosine, glutamine, or alanine
  MISC_FEATURE (4)..(12): each sequence of GGX may be present or absent
  MISC_FEATURE (13)..(20): each sequence of GA may be present or absent
  MISC_FEATURE (22)..(26): each A may be present or absent
GGXGGXGGXGGXGAGAGAGAAAAAAA

SEQ ID NO: 42 - protein, 8 aa, Artificial Sequence
  Chemically synthesized
GCGGGGGG

SEQ ID NO: 43 - protein, 8 aa, Artificial Sequence
  Synthetic modified amino acid sequence
GKGGGGGG

SEQ ID NO: 44 - protein, 14 aa, Artificial Sequence
  Synthetic modified amino acid sequence
GCGGSGGGGSGGGG

SEQ ID NO: 45 - protein, 13 aa, Artificial Sequence
  Chemically synthesized
GKGGGGGGSGGGG

SEQ ID NO: 46 - protein, 13 aa, Artificial Sequence
  Synthetic modified amino acid sequence
GCGGGGGGSGGGG

SEQ ID NO: 47 - protein, 33 aa, Artificial Sequence
  Synthetic modified amino acid sequence
GSSAAAAAAAASGPGQGQGQGQGQGGRPSDTYG

SEQ ID NO: 48 - protein, 747 aa, Araneus diadematus
QGAGAAAAAAGGAGQGGYGGLGGQGAGQGGYGGLGGQGAGQGAGAAAAAAAGGAGQGGYGGLGSQGAGRGGQGAGAAAAAAGGAGQGGYGGLGSQGAGRGGLGGQGAGAAAAAAAGGAGQGGYGGLGNQGAGRGGQGAAAAAAGGAGQGGYGGLGSQGAGRGGLGGQGAGAAAAAAGGAGQGGYGGLGGQGAGQGGYGGLGSQGAGRGGLGGQGAGAAAAAAAGGAGQGGLGGQGAGQGAGASAAAAGGAGQGGYGGLGSQGAGRGGEGAGAAAAAAGGAGQGGYGGLGGQGAGQGGYGGLGSQGAGRGGLGGQGAGAAAAGGAGQGGLGGQGAGQGAGAAAAAAGGAGQGGYGGLGSQGAGRGGLGGQGAGAVAAAAAGGAGQGGYGGLGSQGAGRGGQGAGAAAAAAGGAGQRGYGGLGNQGAGRGGLGGQGAGAAAAAAAGGAGQGGYGGLGNQGAGRGGQGAAAAAGGAGQGGYGGLGSQGAGRGGQGAGAAAAAAVGAGQEGIRGQGAGQGGYGGLGSQGSGRGGLGGQGAGAAAAAAGGAGQGGLGGQGAGQGAGAAAAAAGGVRQGGYGGLGSQGAGRGGQGAGAAAAAAGGAGQGGYGGLGGQGVGRGGLGGQGAGAAAAGGAGQGGYGGVGSGASAASAAASRLSSPQASSRVSSAVSNLVASGPTNSAALSSTISNVVSQIGASNPGLSGCDVLIQALLEVVSALIQILGSSSIGQVNYGSAGQATQIVGQSVYQAL

SEQ ID NO: 49 - protein, 627 aa, Araneus diadematus
PGGYGPGQQGPGGYGPGQQGPSGPGSAAAAAAAAAAGPGGYGPGQQGPGGYGPGQQGPGRYGPGQQGPSGPGSAAAAAAGSGQQGPGGYGPRQQGPGGYGQGQQGPSGPGSAAAASAAASAESGQQGPGGYGPGQQGPGGYGPGQQGPGGYGPGQQGPSGPGSAAAAAAAASGPGQQGPGGYGPGQQGPGGYGPGQQGPSGPGSAAAAAAAASGPGQQGPGGYGPGQQGPGGYGPGQQGLSGPGSAAAAAAAGPGQQGPGGYGPGQQGPSGPGSAAAAAAAAAGPGGYGPGQQGPGGYGPGQQGPSGAGSAAAAAAAGPGQQGLGGYGPGQQGPGGYGPGQQGPGGYGPGSASAAAAAAGPGQQGPGGYGPGQQGPSGPGSASAAAAAAAAGPGGYGPGQQGPGGYAPGQQGPSGPGSASAAAAAAAAGPGGYGPGQQGPGGYAPGQQGPSGPGSAAAAAAAAAGPGGYGPAQQGPSGPGIAASAASAGPGGYGPAQQGPAGYGPGSAVAASAGAGSAGYGPGSQASAAASRLASPDSGARVASAVSNLVSSGPTSSAALSSVISNAVSQIGASNPGLSGCDVLIQALLEIVSACVTILSSSSIGQVNYGAASQFAQVVGQSVLSAF

SEQ ID NO: 50 - protein, 636 aa, Araneus diadematus
ARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 51 - protein, 410 aa, Araneus diadematus
AGSSAAAAAAASGSGGYGPENQGPSGPVAYGPGGPVSSAAAAAAAGSGPGGYGPENQGPSGPGGYGPGGSGSSAAAAAAAASGPGGYGPGSQGPSGPGGSGGYGPGSQGASGPGGPGASAAAAAAAAAASGPGGYGPGSQGPSGPGAYGPGGPGSSAAAAAAAASGPGGYGPGSQGPSGPGVYGPGGPGSSAAAAAAAGSGPGGYGPENQGPSGPGGYGPGGSGSSAAAAAAAASGPGGYGPGSQGPSGPGGSGGYGPGSQGGSGPGASAAAAAAAASGPGGYGPGSQGPSGPGYQGPSGPGAYGPSPSASASVAASVYLRLQPRLEVSSAVSSLVSSGPTNGAAVSGALNSLVSQISASNPGLSGCDALVQALLELVSALVAILSSASIGQVNVSSVSQSTQMISQALS

SEQ ID NO: 52 - protein, 50 aa, Araneus diadematus
SGCDVLVQALLEVVSALVSILGSSSIGQINYGASAQYTQMVGQSVAQALA

SEQ ID NO: 53 - protein, 30 aa, Araneus diadematus
SGCDVLVQALLEVVSALVSILGSSSIGQIN

SEQ ID NO: 54 - protein, 21 aa, Araneus diadematus
SGCDVLVQALLEVVSALVSIL

SEQ ID NO: 55 - protein, 96 aa, Artificial Sequence
  Synthetic recombinant silk protein repetitive fragment
GAGAGSGAGAGSGAGAGSGAGAGSGAGAGSGAGAGSGAGAGSGAGAGSGAGAGSGAGAGSGAGAGSGAGAGSGAGAGSGAGAGSGAGAGSGAGAGS

SEQ ID NO: 56 - protein, 140 aa, Artificial Sequence
  Synthetic recombinant silk protein repetitive fragment
GAGAGSGAGAGSGAGAGSGAGAGSGAGAGSGAGAGSGAGAGSGAGAGSGAGAGSGAGAGSGAGAGSGAGAGSGAGAGSGAGAGSGAGAGSGAGAGSSGFGPVANGGSGEASSESDFGSSGFGPVANASSGEASSESDFAG

SEQ ID NO: 57 - protein, 184 aa, Artificial Sequence
  Synthetic recombinant silk protein repetitive fragment
GAGAGSGAGAGSGAGAGSGAGAGSGAGAGSGAGAGSGAGAGSGAGAGSGAGAGSGAGAGSGAGAGSGAGAGSGAGAGSGAGAGSGAGAGSGAGAGSSGFGPVANGGSGEASSESDFGSSGFGPVANASSGEASSESDFAGSGFGPVANGGSGEASSESDFGSSGFGPVANASSGEASSESDFAG

SEQ ID NO: 58 - protein, 228 aa, Artificial Sequence
  Synthetic recombinant silk protein repetitive fragment
GAGAGSGAGAGSGAGAGSGAGAGSGAGAGSGAGAGSGAGAGSGAGAGSGAGAGSGAGAGSGAGAGSGAGAGSGAGAGSGAGAGSGAGAGSGAGAGSSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAG

SEQ ID NO: 59 - protein, 272 aa, Artificial Sequence
  Synthetic recombinant silk protein repetitive fragment
GAGAGSGAGAGSGAGAGSGAGAGSGAGAGSGAGAGSGAGAGSGAGAGSGAGAGSGAGAGSGAGAGSGAGAGSGAGAGSGAGAGSGAGAGSGAGAGSSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAG

SEQ ID NO: 60 - protein, 448 aa, Artificial Sequence
  Synthetic recombinant silk protein repetitive fragment
GAGAGSGAGAGSGAGAGSGAGAGSGAGAGSGAGAGSGAGAGSGAGAGSGAGAGSGAGAGSGAGAGSGAGAGSGAGAGSGAGAGSGAGAGSGAGAGSSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAG

SEQ ID NO: 61 - protein, 624 aa, Artificial Sequence
  Synthetic recombinant silk protein repetitive fragment
GAGAGSGAGAGSGAGAGSGAGAGSGAGAGSGAGAGSGAGAGSGAGAGSGAGAGSGAGAGSGAGAGSGAGAGSGAGAGSGAGAGSGAGAGSGAGAGSSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAGSGFGPVANGGSGEASSESDFGSSGFGPVANASSGEASSESDFAG

SEQ ID NO: 62 - protein, 124 aa, Artificial Sequence
  Based on ADF-3
GAASAAVSVGGYGPQSSSAPVASAAASRLSSPAASSRVSSAVSSLVSSGPTNQAALSNTISSVVSQVSASNPGLSGCDVLVQALLEVVSALVSILGSSSIGQINYGASAQYTQMVGQSVAQALA

SEQ ID NO: 63 - protein, 109 aa, Artificial Sequence
  Based on ADF-3
GAYGPSPSASASVAASRLSSPAASSRVSSAVSSLVSSGPTNGAAVSGALNSLVSQISASNPGLSGCDALVQALLELVSALVAILSSASIGQVNVSSVSQSTQMISQALS

SEQ ID NO: 64 - protein, 576 aa, Artificial Sequence
  Synthetic spider silk C16 protein
MASMTGGQQMGRGSMGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPG

SEQ ID NO: 65 - protein, 1154 aa, Artificial Sequence
  Recombinant spider silk protein ADF3Kai-Large- NRSH1
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVSRARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSIL

SEQ ID NO: 66 - protein, 24 aa, Artificial Sequence
  His tag and start codon
MHHHHHHHHHHSSGSSLEVLFQGP

SEQ ID NO: 67 - protein, 434 aa, Artificial Sequence
  Recombinant spider silk protein ADF4Kai
MHHHHHHHHHHSSGSSLEVLFQGPAGSSAAAAAAASGSGGYGPENQGPSGPVAYGPGGPVSSAAAAAAAGSGPGGYGPENQGPSGPGGYGPGGSGSSAAAAAAAASGPGGYGPGSQGPSGPGGSGGYGPGSQGASGPGGPGASAAAAAAAAAASGPGGYGPGSQGPSGPGAYGPGGPGSSAAAAAAAASGPGGYGPGSQGPSGPGVYGPGGPGSSAAAAAAAGSGPGGYGPENQGPSGPGGYGPGGSGSSAAAAAAAASGPGGYGPGSQGPSGPGGSGGYGPGSQGGSGPGASAAAAAAAASGPGGYGPGSQGPSGPGYQGPSGPGAYGPSPSASASVAASVYLRLQPRLEVSSAVSSLVSSGPTNGAAVSGALNSLVSQISASNPGLSGCDALVQALLELVSALVAILSSASIGQVNVSSVSQSTQMISQALS

SEQ ID NO: 68 - protein, 355 aa, Artificial Sequence
  Recombinant spider silk protein MaSp2_N
MHHHHHHSSGSSLEVLFQGPARAGPGGYRPGQQGPSGPGSAAAAAAAAAGPGGYGPGQQGPGGYGPGQQGPSGAGSAAAAAAAGPGQQGLGGYGPGQQGPGGYGPGQQGPGGYGPGSASAAAAAAGPGQQGPGGYGPGQQGPSGPGSASAAAAAAGPGGYGPGQQGPGGYAPGQQGPSGPGSAAAAAAAARAGPGGYGPAQQGPSGPGIAASAASAGPGGYGPAQQGPAGYGPGSAVAASAGAGSAGYGPGSQASAAASRLASPDSGARVASAVSNLVSSGPTSSAALSSVISNAVSQIGASNPGLSGCDVLIQALLEIVSACVTILSSSSIGQVNYGAASQFAQVVGQSVLSAF

SEQ ID NO: 69 - protein, 5 aa, Artificial Sequence
  Synthetic recombinant silk protein repeating fragment
  MISC_FEATURE (5)..(5): X can be alanine, serine, tyrosine, or valine
GPGGX

SEQ ID NO: 70 - protein, 559 aa, Artificial Sequence
  Recombinant spider silk protein Flag_92_short2
MHHHHHHHHHHSSGSSLEVLFQGPGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGPGGSYGPGGAGGPYGPGGPYGPGGEGPGGAGGPYGPGGVGPGGGGPGGYGPGGAGPGGYGPGGSGPGGYGPGGSGPGGYGPGGSGPGGYGPGGSGPGGYGPGGSGPGGSGPGGYGPGGSGPGGSGPGGYGPGGSGPGGYGPGGSGPGGSGPGGYGPGGSGPGGSGPGGYGPGGSGPGGFGPGGFGPGGSGPGGYGPGGSGPGGAGPGGVGPGGFGPGGAGPGGAGPGGAGPGGAGPGGAGPGGAGPGGAGPGGAGPGGAGGAGGAGGAGGSGGAGGSGGTTIIEDLDITIDGADGPITISEELTISAYYPSSRVPDMVNGIMSAMQGSGFNYQMFGNMLSQYSSGSGTCNPNNVNVLMDALLAALHCLSNHGSSSFAPSPTPAAMSAYSNSVGRMFAY

(19) United States
(12) Patent Application Publication
(10) Pub. No.: US 2022/0287944 A1
(43) Pub. Date: Sep. 15, 2022

(54) SILK PERSONAL CARE COMPOSITIONS

(71) Applicant: Evolved by Nature, Inc., Medford, MA (US)

(72) Inventors: Marius COSTACHE, Lexington, MA (US); Eva GENEL, Medford, MA (US); Abigail GIARROSSO, Methuen, MA (US); Laura R. MUOLLO, Dracut, MA (US); Gregory H. ALTMAN, Providence, RI (US); Carlos J. BOSQUES, Arlington, MA (US); Rose MAXWELL, Riverdale, NY (US); Samiul AMIN, Riverdale, NY (US)

(21) Appl. No.: 17/636,716

(22) PCT Filed: Aug. 20, 2020
(86) PCT No.: PCT/US2020/047265

§ 371 Date: Feb. 18, 2022

Related U.S. Application Data
(60) Provisional application No. 62/889,350, filed on Aug. 20, 2019.
(60) Provisional application No. 62/902,577, filed on Sep. 19, 2019.
(60) Provisional application No. 62/935,806, filed on Nov. 15, 2019.
(60) Provisional application No. 63/056,394, filed on Jul. 24, 2020.

(51) Int. Cl. A61K 8/64; A61Q 11/02; A61K 8/19; A61Q 19/10

(52) U.S. Cl. CPC A61K 8/64; A61Q 11/02; A61K 8/19; A61Q 19/10; A61K 2800/48; A61K 2800/92

(57) ABSTRACT

This disclosure is in the field of novel surfactant blend of silk fibroin protein fragments and a natural surfactant and personal care compositions and products thereof.

### CROSS REFERENCE TO RELATED APPLICATIONS

This application claims the benefit of U.S. Provisional Patent Application No. 62/889,350 filed Aug. 20, 2019, and U.S. Provisional Patent Application No. 62/902,577 filed Sep. 19, 2019, and U.S. Provisional Patent Application No. 62/935,806 filed Nov. 15, 2019, and U.S. Provisional Patent Application No. 63/056,394 filed Jul. 24, 2020, all of which are incorporated by reference in their entireties.

### REFERENCE TO SEQUENCE LISTING SUBMITTED ELECTRONICALLY

The sequence listing contained in the file named “EBN_5017WO_SEQ_ST25.txt”, created on Aug. 20, 2020, and having a size of 83.8 kilobytes, has been submitted electronically herewith via EFS-Web, and the contents of the txt file are hereby incorporated by reference in their entirety.

### FIELD

This disclosure is in the field of novel surfactant blend of silk fibroin protein fragments and at least one natural surfactant and personal care compositions and products thereof. The natural surfactant improves the surface-active properties of the silk fibroin protein fragments including surface tension, interfacial tension, etc. The silk personal care compositions provide benefits to skin such as promoting cell repair and regeneration, reducing transdermal water loss, boosting collagen level, alleviating sun damage, gentle skin exfoliation, skin tightening, reducing and improving scar's appearance, reducing skin inflammation, rendering high gloss, ultraviolet light protection, and the like.

### BACKGROUND

Personal care compositions such as oral care compositions, skin care compositions are used for a wide variety of purposes such as enhancing personal health, hygiene, and appearance, preventing and treating a variety of diseases, and other conditions in humans and in animals. The formulations of such compositions present a number of challenges. They must be cosmetically acceptable for their intended use. Compositions containing cosmetically functional materials must deliver the materials to the desired locations including oral cavity, skin, or hair at effective amount under the typical use conditions by the consumers. Moreover, aesthetic appeal of all such compositions is important, and play an important role in consumer acceptance of many personal care products. Many of the products on the markets are deficient in providing both the aesthetic appeal and the effective delivery of cosmetic benefits. Thus, there is an ongoing need for new personal care compositions and methods of their use.

### SUMMARY

In an embodiment, this disclosure provides a silk personal care composition comprising silk fibroin fragments having an average weight average molecular weight selected from between about 1 kDa to about 5 kDa, from between about 5 kDa to about 10 kDa, from between about 6 kDa to about 17 kDa, from between about 10 kDa to about 15 kDa, from between about 15 kDa to about 20 kDa, from between about 17 kDa to about 39 kDa, from between about 20 kDa to about 25 kDa, from between about 25 kDa to about 30 kDa, from between about 30 kDa to about 35 kDa, from between about 35 kDa to about 40 kDa, from between about 39 kDa to about 80 kDa, from between about 40 kDa to about 45 kDa, from between about 45 kDa to about 50 kDa, from between about 60 kDa to about 100 kDa, and from between about 80 kDa to about 144 kDa, a polydispersity ranging from about 1 to about 5; from 0 to 500 ppm lithium bromide; from 0 to 500 ppm sodium carbonate; and a carrier. In some embodiments, the silk fibroin fragments of the silk personal care composition is in the form of an aqueous solution (silk solution). In some embodiments, the silk personal care composition further comprise silk amino acids resulted from the hydrolysis of silk of Bombyx mori and silk powders resulted from drying of the silk solution. In some embodiments, the silk fibroin fragments have a polydispersity ranging from 1.0 to about 1.5. In some embodiments, the silk fibroin fragments have a polydispersity ranging from about 1.5 to about 2.0. In some embodiments, the silk fibroin fragments have a polydispersity ranging from about 1.5 to about 3.0. In some embodiments, the silk fibroin fragments have a polydispersity ranging from about 2.0 to about 2.5. In some embodiments, the silk fibroin fragments have a polydispersity ranging from about 2.5 to about 3.0. In some embodiments, the silk fibroin fragments have a polydispersity ranging from about 3.0 to about 3.5. In some embodiments, the silk fibroin fragments have a polydispersity ranging from about 3.5 to about 4.0. In some embodiments, the silk fibroin fragments have a polydispersity ranging from about 4.0 to about 4.5. In some embodiments, the silk fibroin fragments have a polydispersity ranging from about 4.5 to about 5.0. In some embodiments, the silk fibroin fragments are present at an amount ranging from about 0.01 wt. % to about 10.0 wt. % by the total weight of the silk personal care composition. In some embodiments, the silk fibroin fragments are present at an amount ranging from about 0.01 wt. % to about 1.0 wt. % by the total weight of the silk personal care composition. In some embodiments, the silk fibroin fragments are present at an amount ranging from about 1.0 wt. % to about 2.0 wt. % by the total weight of the silk personal care composition. In some embodiments, the silk fibroin fragments are present at an amount ranging from about 2.0 wt. % to about 3.0 wt. % by the total weight of the silk personal care composition. In some embodiments, the silk fibroin fragments are present at an amount ranging from about 3.0 wt. % to about 4.0 wt. % by the total weight of the silk personal care composition. In some embodiments, the silk fibroin fragments are present at an amount ranging from about 4.0 wt. % to about 5.0 wt. % by the total weight of the silk personal care composition. In some embodiments, the silk fibroin fragments are present at an amount ranging from about 5.0 wt. % to about 6.0 wt. % by the total weight of the silk personal care composition. In some embodiments, the silk personal care composition further comprises about 0.01% (w/w) to about 10% (w/w) sericin by the total weight of the silk personal care composition. In some embodiments, the silk fibroin fragments in the silk personal care composition do not spontaneously or gradually gelate and do not visibly change in color or turbidity when in an aqueous solution for at least 10 days prior to be formulated into the silk personal care composition. In some embodiments, the carrier comprises an oil phase. In some embodiments, the carrier comprises an aqueous phase. In some embodiments, the silk personal care composition further comprising an emulsifier. In some embodiments, the silk personal care composition comprises an “oil-in-water” type emulsion or a “water-in-oil” type emulsion.

In some embodiments, the silk personal care composition forms an oral care composition. In some embodiments, the oral care composition further comprises an additive selected from the group consisting of a filler, a diluent, a remineralizing agent, an anti-calculus agent, an anti-plaque agent, a buffer, an abrasive, an alkali metal bicarbonate salt, a binder, a thickening agent, a humectant, a whitening agent, a bleaching agent, a stain removing agent, a surfactant, titanium dioxide, a flavoring agent, xylitol, a coloring agent, a foaming agent, a sweetener, an antibacterial agent, a preservative, a vitamin, a pH-adjusting agent, an anti-caries agent, a desensitizing agent, a coolant, a salivating agent, a warming agent, a numbing agent, a chelating agent, and combinations thereof. In some embodiments, the oral care composition is formulated as a product selected from the group consisting of a toothpaste, a dentifrice, a tooth powder, an oral gel, an aqueous gel, a non-aqueous gel, a mouth rinse, a mouth spray, a plaque removing liquid, a denture product, a dental solution, a lozenge, an oral tablet, a chewing gum, a candy, a fast-dissolving film, a strip, a dental floss, a tooth glossing product, a finishing product, and an impregnated dental implement. In some embodiments, the oral care composition is formulated as a toothpaste comprising a tooth care active agent selected from the group consisting an abrasive, an anti-calculus agent, an anti-plaque agent, a humectant, a whitening agent, an anti-caries agent, a desensitizing agent, a coolant, a salivating agent, a warming agent, a numbing agent, and combinations thereof. In some embodiments, the oral care composition is formulated as a tooth remineralization product comprising a therapeutically effective amount of a remineralizing agent. In some embodiments, the remineralizing agent is selected from the group consisting of fluoride, calcium source compound, phosphate source compound, calcium carbonate, sodium hydrogen phosphate, sodium dihydrogen phosphate, potassium hydrogen phosphate, potassium dihydrogen phosphate, amorphous calcium phosphate (ACP), tricalcium phosphate, casein phosphoprotein-ACP, bioactive glass, calcium sodium phosphosilicate, arginine bicarbonate-calcium carbonate complex, and combinations thereof. In some embodiments, the tooth remineralization composition is formulated as a product selected from the group consisting of remineralizing gel, a remineralizing mouthwash, a remineralizing tooth powder, a remineralizing chewing gums, a remineralizing lozenge, and a remineralizing toothpaste.

In some embodiments, the silk personal care composition is formulated as a skin cleansing composition. In some embodiments, the skin cleansing composition further comprises an additive selected from the group consisting of a cleansing surfactant, a soap base, a detergent, a lathering surfactant, a skin conditioning agent, an oil control agent, an anti-acne agent, an astringent, an exfoliating particle or agent, a skin calming agent, a plant extract, an essential oil, a coolant, a humectant, a moisturizer, a structurant, a gelling agent, an antioxidant, an anti-aging compound, a skin lightening agent, a preservative, a filler, a fragrance, a thickener, a coloring agent, an antimicrobial agent, and combinations thereof. In some embodiments, the skin cleansing composition is formulated as a product selected from the group consisting of a cleansing lotion, a cleansing milk, a cleansing gel, a cleansing soap bar, an exfoliating product, a bath and shower soap in bar, a body wash, a hand wash, a cleansing wipe, a cleansing pad, and a bath product.

In some embodiments, the silk personal care composition is formulated as a makeup composition. In some embodiments, the makeup composition further comprises a cosmetic ingredient selected from the group consisting of an oil control agent, a plant extract, an essential oil, a humectant, a moisturizer, a structurant, a gelling agent, an antioxidant, an anti-aging compound, a sunscreen, a skin lightening agent, a sequestering agent, a preservative, a filler, a fragrance, a thickener, a wetting agent, a coloring agent, a cosmetic powder, and a combination thereof. In some embodiments, the makeup composition is formulated as a product selected from the group consisting of a color cosmetic, a mascara, a lipstick, a lip liner, an eye shadow, an eye-liner, a rouge, a face powder, a foundation, and a blush.

In some embodiments, the silk personal care composition is formulated as a cosmetic composition and the carrier is a cosmetically acceptable medium. In some embodiments, the cosmetic composition further comprises a cosmetic ingredient selected from the group consisting of a surfactant, a skin conditioning agent, an oil control agent, an anti-acne agent, an astringent, an exfoliating particle or agent, a skin calming agent, a plant extract, an essential oil, a coolant, a humectant, a moisturizer, a structurant, a gelling agent, an antioxidant, an anti-aging compound, a sunscreen, a skin lightening agent, a sequestering agent, a preservative, a filler, a fragrance, a thickener, a wetting agent, a coloring agent, a glitter, and combinations thereof. In some embodiments, the cosmetic composition is formulated as a product selected from the group consisting of a cream, an emulsion, a foam, an ointment, a lotion, a liquid, a hydrogel, a shaving or after-shave cream, a conditioner, a cologne, a shaving or after-shave lotion, a perfume, a cosmetic oil, a facial mask, a moisturizer, an anti-wrinkle treatment cream, an eye treatment lotion, a tanning cream, a tanning lotion, a tanning emulsion, a sunscreen cream, a sunscreen lotion, a sunscreen emulsion, a tanning oil, a sunscreen oil, a hand lotion, a tonic, and a body lotion.

In some embodiments, the silk personal care composition is formulated as a deodorant or antiperspirant composition and the carrier is a dermatologically acceptable medium. In some embodiments, the deodorant or antiperspirant composition further comprises an additive selected from the group consisting of a deodorant active, an antiperspirant active, an emollient, a humectant, a moisturizer, an astringent, an antiseptic agent, a gellant, a surfactant, a thickening agent, a cosmetic powder, a fragrance, an antimicrobial agent, a preservative, a coloring agent, a filler, a co-emulsifier, a hardener, a strengthener, a chelating agent, a thixotropic agent, an oil absorbing agent, an antioxidant, and combinations thereof. In some embodiments, the deodorant or antiperspirant composition is formulated as a product selected from the group consisting of a stick, a roll-on, a powder, a gel, an aerosol, a paste, and a cream. In some embodiments, the deodorant or antiperspirant composition is clear, transparent, or translucent.

In some embodiments, the silk personal care composition is formulated as a nail care composition and the carrier is a dermatologically acceptable medium. In some embodiments, the nail care composition further comprises an additive selected from the group consisting of a film-forming agent, a suspending agent, a thixotropic agent, a coloring agent, a pigment, a glitter, a plasticizer, a thickening agent, a nail hydrating agent, a nail hardening agent, boric acid, a vitamin, a plant extract, an essential oil, a preservative, a mineral salt, a fruit extract, an algae extract, a fungus extract, a caviar extract, a vegetable oil, an amino acid, a peptide, a protein, a ceramide, allantoin or an allantoin derivative, an organosilicon derivative, an antioxidant, a UV light absorber, a moisturizer, a stabilizer, a fragrance, a micronutrient, a solvent, and combinations thereof. In some embodiments, the nail care composition is formulated as a product selected from the group consisting of a nail polish, and a nail polish remover.

In an embodiment, this disclosure provides a silk fibroin fragment composition comprising silk fibroin fragments having an average weight average molecular weight selected from between about 1 kDa to about 5 kDa, from between about 5 kDa to about 10 kDa, from between about 6 kDa to about 17 kDa, from between about 10 kDa to about 15 kDa, from between about 15 kDa to about 20 kDa, from between about 17 kDa to about 39 kDa, from between about 20 kDa to about 25 kDa, from between about 25 kDa to about 30 kDa, from between about 30 kDa to about 35 kDa, from between about 35 kDa to about 40 kDa, from between about 39 kDa to about 80 kDa, from between about 40 kDa to about 45 kDa, from between about 45 kDa to about 50 kDa, from between about 60 kDa to about 100 kDa, and from between about 80 kDa to about 144 kDa, and a polydispersity ranging from 1 to about 5, from 0 to 500 ppm lithium bromide, from 0 to 500 ppm sodium carbonate; and at least one emulsifiable component. In some embodiments, the silk fibroin fragments have a polydispersity ranging from 1 to about 1.5. In some embodiments, the silk fibroin fragments have a polydispersity ranging from about 1.5 to about 2.0. In some embodiments, the silk fibroin fragments have a polydispersity ranging from about 1.5 to about 3.0. In some embodiments, the silk fibroin fragments have a polydispersity ranging from about 2.0 to about 2.5. In some embodiments, the silk fibroin fragments have a polydispersity ranging from about 2.5 to about 3.0. In some embodiments, the silk fibroin fragments have a polydispersity ranging from about 3.0 to about 3.5. In some embodiments, the silk fibroin fragments have a polydispersity ranging from about 3.5 to about 4.0. In some embodiments, the silk fibroin fragments have a polydispersity ranging from about 4.0 to about 4.5. In some embodiments, the silk fibroin fragments have a polydispersity ranging from about 4.5 to about 5.0. In some embodiments, the silk fibroin fragments are present at an amount ranging from about 0.01 wt. % to about 10.0 wt. % by the total weight of the silk fibroin fragment composition. In some embodiments, the silk fibroin fragments are present at an amount ranging from at about 0.01 wt. % to about 1.0 wt. % by the total weight of the silk fibroin fragment composition. In some embodiments, the silk fibroin fragments are present at an amount ranging from at about 1.0 wt. % to about 2.0 wt. % by the total weight of the silk fibroin composition. In some embodiments, the silk fibroin fragments are present at an amount ranging from about 2.0 wt. % to about 3.0 wt. % by the total weight of the silk fibroin fragment composition. In some embodiments, the silk fibroin fragments are present at an amount ranging from about 3.0 wt. % to about 4.0 wt. % by the total weight of the silk fibroin fragment composition. In some embodiments, the silk fibroin fragments are present at an amount ranging from about 4.0 wt. % to about 5.0 wt. % by the total weight of the silk fibroin fragment composition. In some embodiments, the silk fibroin fragments are present at an amount ranging from about 5.0 wt. % to about 6.0 wt. % by the total weight of the silk fibroin fragment composition. In some embodiments, the silk fibroin fragment composition further comprising about 0.01% (w/w) to about 10% (w/w) sericin by the total weight of the silk fibroin fragment composition. In some embodiments, the silk fibroin fragment composition do not spontaneously or gradually gelate and do not visibly change in color or turbidity when in an aqueous solution for at least 10 days prior to be formulated into the silk fibroin fragment composition. In some embodiments, the silk fibroin fragment composition further comprises an additive selected from the group consisting of butanediol, propanediol, ethanediol, glycerol, butantetraol, xylitol, D-sorbitol, inositol, polyethylene glycol, hydroxyethyl cellulose, hydroxypropyl methylcellulose, dextran, gelatin, carboxymethyl cellulose, propylene glycol, polysorbate 80, polyvinyl alcohol, povidone, saponin, sucrose, fructose, maltose, carrageenan, chitosan, alginate, hyaluronic acid, and combinations thereof. In some embodiments, the silk fibroin composition comprises one or more solvent selected from the group consisting of methanol, ethanol, propanol, isopropanol, acetonitrile, and combinations thereof. In some embodiments, the emulsifiable component comprises a hydrophobic emulsifiable component, a hydrophilic emulsifiable component, or both. In some embodiments, the emulsifiable component comprises a hydrophobic emulsifiable component. In some embodiments, the hydrophobic emulsifiable component is selected from the group consisting of oil, fat, wax, lipid, and combinations thereof. In some embodiments, the oil of the hydrophobic emulsifiable component is selected from the group consisting of hydrocarbon oil, mineral oil, silicon oil, fatty acid having 8 to 32 carbon atoms, fatty alcohol having 8 to 32 carbon atoms, synthetic ester oil derived from the esterification product of fatty acid having 8 to 32 carbon atoms and an alcohol, fatty acid glyceride, glyceryl trioctanoate, glyceryl triisopalmitate, cholesteryl isostearate, isopropyl palmitate, isopropyl myristate, neopentyl glycol dicaprate, isopropyl isostearate, octadecyl myristate, cetyl 2-ethylhexanoate, cetearyl isononanoate, cetearyl octanoate, isononyl isononanoate, isotridecyl isononanoate, glyceryl tri-2-ethylhexanoate, glyceryl tri(caprylatelcaprate), diethylene glycol monoethyl ether oleate, dicaprylyl ether, caprylic acid/capric acid propylene glycol diester, isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexylate, dipentaerythritol fatty acid ester, N-alkyl glycol monoisostearate, neopentyl glycol dicaprate, diisostearyl malate, glyceryl di-2-heptylundecanoate, trimethylolpropane tri-2-ethylhexylate, trimethylolpropane triisostearate, pentaneerythritol tetra-2-ethylhexylate, glyceryl tri-2-ethylhexylate, trimethylolpropane triisostearate, cetyl 2-ethylhexanoate, 2-ethylhexyl palmitate, glyceryl trimyristate, tri-2-heptylundecanoic glyceride, oleyl oleate, cetostearyl alcohol, 2-heptylundecyl palmitate, diisopropyl adipate, N-lauroyl-L-glutamic acid-2-octyldodecyl ester, di-2-heptylundecyl adipate, ethyl laurate, di-2-ethylhexyl cebatate. 2-hexyldecyl myristate, 2-hexyldecyl palmitate, 2-hexyldecyl adipate, diisopropyl cebatate, 2-ethylhexyl succinate, ethyl acetate, butyl acetate, amyl acetate and triethyl citrate. In some embodiments, the fat of the hydrophobic emulsifiable component is selected from the group consisting of liquid fat, solid fat, avocado oil, tsubaki oil, turtle oil, macademia nut oil, corn oil, mink oil, olive oil, rape seed oil, egg yolk oil, sesame seed oil, persic oil, wheat germ oil, sasanqua oil, castor oil, linseed oil, safflower oil, cotton seed oil, perilla oil, soybean oil, peanut oil, tea seed oil, kaya oil, rice bran oil, Chinese wood oil, Japanese wood oil, jojoba oil, germ oil, sweet almond oil, rosehip seed oil, calendula oil, grape seed oil, apricot kernel oil, flaxseed oil, hazelnut oil, walnut oil, pecan nut oil, sesame oil, emu oil, coconut oil, sunflower oil, canola oil, algae oil, cacao butter, horse tallow, hardened coconut oil, palm oil, beef tallow, sheep tallow, pork tallow, hardened beef tallow, palm kernel oil, Japanese core wax, hydrogenated castor oil, and combinations thereof. In some embodiments, the wax of the hydrophobic emulsifiable component is selected from the group consisting of butter, petrolatum, polyethylene wax, polypropylene wax, Japanese wax, beeswax, candelilla wax, paraffin wax, ozokerite, microcrystalline wax, carnauba wax, cotton wax, esparto wax, bayberry wax, tree wax, whale wax, montan wax, bran wax, lanolin wax, kapok wax, lanolin acetate, sugar cane wax, lanolin fatty acid isopropyl ester, hexyl laurate, reduced lanolin, jojoba wax, hard lanolin, shellac wax, POE lanolin alcohol ether, lanolin alcohols with 40 mols. ethylene oxide, lanolin alcohols with 65-70 mols. ethylene oxide, POE lanolin alcohol acetate, POE cholesterol ether, lanolin fatty acid, POE hydrogenated lanolin alcohol ether, and combinations thereof. In some embodiments, the lipid of the hydrophobic emulsifiable component is selected from the group consisting of phospholipid, polymer-lipid conjugate, carbohydrate-lipid conjugate, dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-dimyristoyl-sn-glycero-3-phosphorylglycerol (DMPG); 1,2-distearoyl-sn-glycero-3-phosphoethanolamine (DSPE); 1,2-dioleoyl-sn-glycero-3-phosphocholine (DOPC); 1,2-dioleoyl-sn-glycero-3-phosphoethanolamine (DOPE); 1,2-dipalmitoyl-sn-glycero-3-phospho-(1′-rac-glycerol) (DPPG); 1,2-distearoyl-sn-glycero-3-phosphocholine (DSPC), distearoylphosphoethanolamine conjugated with polyethylene glycol (DSPE-PEG); phosphatidylserine (PS), phosphatidylethanolamine (PE), phosphatidylglycerol (PG), phosphatidylcholine (PC), cholesterol, 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-diarachidyl-sn-glycero-3-phosphoethanolamine (20:0 PE), 1-stearoyl-2-linoleoyl-sn-glycero-3-phosphoethanolamine, 1,2-diheptadecanoyl-sn-glycero-3-phosphocholine (17:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof. In some embodiments, the lipid is a phospholipid selected from soy lecithin and egg lecithin.

In some embodiments, the silk fibroin fragment composition further comprises a thickening agent or gelling agent selected from the group consisting of hydroxyethyl cellulose, hydroxypropyl methylcellulose, dextran, gelatin, carboxymethyl cellulose, propylene glycol, polysorbate 80, polyvinyl alcohol, povidone, sucrose, fructose, maltose, carrageenan, chitosan, alginate, hyaluronic acid, gum arabic, xanthan gum galactomannans, pectin, and combinations thereof.

In some embodiments, the silk fibroin fragment composition further comprises a density matching agent (also known as weighting agent) selected from the group consisting of ester gum (EG), damar gum (DG), sucrose acetate isobutyrate (SAIB), brominated vegetable oil (BVO), and combinations thereof. In some embodiments, the weighting agent concentrations required to match the oil and aqueous phase densities is of about 10.0 wt. % to about 25.0 wt. % for BVO, about 35.0 wt. % to about 55.0 wt. % for EG, about 35.0 wt. % to about 55.0 wt. % for DG, and about 25.0 wt. % to about 45.0 wt. % for SAIB.

In some embodiments, the silk fibroin fragment composition has a hydrophilic-lipophilic balance (HLB) value selected from the group consisting of from 0 to about 3, from about 3 to about 6, from about 6 to about 9, from about 9 to about 12, from about 12 to about 15, from about 15 to about 18, and greater than 18. In some embodiments, the silk fibroin fragment composition has a HLB value selected from the group consisting of 0, about 1, about 2, about 3, about 4, about 5, about 6, about 7, about 8, about 9, about 10, about 11, about 12, about 13, about 14, about 15, about 16, about 17, about 18, about 19, and about 20. In some embodiments, the silk fibroin fragment composition has a HLB value ranging from about 8 to about 18. In some embodiments, the silk fibroin fragment composition has a HLB value ranging from 0 to about 8.

In some embodiments, the silk fibroin fragment composition further comprises about 0.1 wt. % to about 5.0 wt. % of a natural surfactant having a HLB value of about 0 to about 6, wherein the silk fibroin fragment composition forms oil-in-water emulsion containing about 0.8 wt. % to about 10.0 wt. % silk fibroin fragments, wherein the wt. % is relative to the total weight of the silk fibroin fragment composition. In some embodiments, the natural surfactant is capable of forming a gel network in a continuous aqueous phase. In some embodiments, the natural surfactant is selected from the group consisting of sucrose ester, cetearyl glucoside, caprylyl/capryl glucoside, sucrose laurate, sucrose palmitate, sucrose stearate, sucrose cocoate, sorbitan monostearate, and combinations thereof.

In some embodiments, the silk fibroin fragment composition is suitable for formation of an “oil-in-water” type emulsion. In some embodiments, the silk fibroin fragment composition is suitable for formation of a “water-in-oil” type emulsion.

In an embodiment, this disclosure provides a silk personal care composition comprising the silk fibroin fragment composition and one or more personal care active ingredients, wherein the silk personal care composition is formulated as an oral care composition, a skin care composition, a hair care composition, a cosmetic composition, a makeup composition, a sun care composition, a deodorant, an antiperspirant composition, a nail cosmetic composition, a skin cleansing composition, an aromatic cosmetic, or a bath cosmetic composition.

In some embodiments, this disclosure provides a silk personal care product comprising the silk fibroin fragment composition and one or more personal care active ingredients, wherein the silk personal care product is selected from the group consisting of a beauty soap, a soap bar, a facial wash, a hand wash, a body wash, a cleansing wipe, a cleansing pad, a cleansing foam, a rinse, a cleansing lotion, a cleansing milk, a cleansing gel, a cleansing soap bar, an exfoliating product, a bath and shower soap in bar, a cream, an emulsion, a shaving or after-shave cream, a foam, a conditioner, a cologne, a shaving or after-shave lotion, a perfume, a cosmetic oil, a facial mask, a moisturizer, an anti-wrinkle, an eye treatment, a tanning cream, a tanning lotion, a tanning emulsion, a sunscreen cream, a sunscreen lotion, a sunscreen emulsion, a tanning oil, a sunscreen oil, a hand lotion, a body lotion, a color cosmetic, a mascara, a lipstick, a lip liner, an eye shadow, an eye-liner, a rouge, a face powder, a foundation, a blush, perfume, bath soap in bar, bath product, a toothpaste, a dentifrice, a tooth powder, an oral gel, an aqueous gel, a non-aqueous gel, a mouth rinse, a mouth spray, a plaque removing liquid, a denture product, a dental solution, a lozenge, oral tablet, a chewing gum, a candy, a fast-dissolving film, a strip, a dental floss, a tooth glossing product, a finishing product, an impregnated dental implement, a remineralizing gel, a remineralizing mouthwash, a remineralizing tooth powder, a remineralizing chewing gum, a remineralizing lozenge, a remineralizing toothpaste, a antiperspirant stick, a roll-on deodorant, a powder deodorant, a gel deodorant, an aerosol deodorant, a paste deodorant, and a cream nail polish, and a nail polish remover.

In an embodiment, this disclosure provides a silk personal care product comprising a substantially solid silk composition comprising silk fibroin fragments having an average weight average molecular weight selected from between about 5 kDa to about 10 kDa, from between about 6 kDa to about 17 kDa, from between about 10 kDa to about 15 kDa, from between about 15 kDa to about 20 kDa, from between about 17 kDa to about 39 kDa, from between about 20 kDa to about 25 kDa, from between about 25 kDa to about 30 kDa, from between about 30 kDa to about 35 kDa, from between about 35 kDa to about 40 kDa, from between about 39 kDa to about 80 kDa, from between about 40 kDa to about 45 kDa, from between about 45 kDa to about 50 kDa, from between about 60 kDa to about 100 kDa, and from between about 80 kDa to about 144 kDa, and a polydispersity between 1 and about 5. In some embodiments, the silk fibroin fragments have a polydispersity between 1 and about 1.5. In some embodiments, the silk fibroin fragments have a polydispersity between about 1.5 and about 2.0. In some embodiments, the silk fibroin fragments have a polydispersity between about 1.5 and about 3.0. In some embodiments, the silk fibroin fragments have a polydispersity between about 2.0 and about 2.5. In some embodiments, the silk fibroin fragments have a polydispersity between about 2.5 and about 3.0. In some embodiments, the silk fibroin fragments have a polydispersity ranging from about 3.0 to about 3.5. In some embodiments, the silk fibroin fragments have a polydispersity ranging from about 3.5 to about 4.0. In some embodiments, the silk fibroin fragments have a polydispersity ranging from about 4.0 to about 4.5. In some embodiments, the silk fibroin fragments have a polydispersity ranging from about 4.5 to about 5.0. In some embodiments, the substantially solid silk composition further comprising about 0.01% (w/w) to about 10% (w/w) sericin relative to the silk fibroin fragments. In some embodiments, the silk fibroin fragments are formulated into particles. In some embodiments, the silk particles have a size of between about 1 μm and about 1000 μm. In some embodiments, the silk fibroin fragments are obtained from a precursor solution comprising silk fibroin fragments having an average weight average molecular weight selected from between about 5 kDa to about 10 kDa, from between about 6 kDa to about 17 kDa, from between about 10 kDa to about 15 kDa, from between about 15 kDa to about 20 kDa, from between about 17 kDa to about 39 kDa, from between about 20 kDa to about 25 kDa, from between about 25 kDa to about 30 kDa, from between about 30 kDa to about 35 kDa, from between about 35 kDa to about 40 kDa, from between about 39 kDa to about 80 kDa, from between about 40 kDa to about 45 kDa, from between about 45 kDa to about 50 kDa, from between about 60 kDa to about 100 kDa, and from between about 80 kDa to about 144 kDa, and a polydispersity between 1 and about 5. In some embodiments, the silk fibroin fragments in the precursor solution have a polydispersity between 1 and about 1.5. In some embodiments, the silk fibroin fragments in the precursor solution have a polydispersity between about 1.5 and about 2.0. In some embodiments, the silk fibroin fragments in the precursor solution have a polydispersity between about 1.5 and about 3.0. In some embodiments, the silk fibroin fragments in the precursor solution have a polydispersity between about 2.0 and about 2.5. In some embodiments, the silk fibroin fragments in the precursor solution have a polydispersity between about 2.5 and about 3.0. In some embodiments, the silk fibroin fragments have a polydispersity ranging from about 3.0 to about 3.5. In some embodiments, the silk fibroin fragments have a polydispersity ranging from about 3.5 to about 4.0. In some embodiments, the silk fibroin fragments have a polydispersity ranging from about 4.0 to about 4.5. In some embodiments, the silk fibroin fragments have a polydispersity ranging from about 4.5 to about 5.0. In some embodiments, the precursor solution further comprises about 0.01% (w/w) to about 10% (w/w) sericin relative to the silk fibroin fragments in the precursor solution. In some embodiments, the silk fibroin fragments in the precursor solution do not spontaneously or gradually gelate and do not visibly change in color or turbidity when in the precursor solution for at least 10 days prior to obtaining the silk fibroin fragments in the substantially solid silk composition. In some embodiments, the silk fibroin fragments are obtained from the precursor solution by a process selected from a lyophilization process, a thin film evaporation process, a salting-out process, and a PVA-assisted method.

In an embodiment, this disclosure provides a mixture comprising the substantially solid silk composition as described herein and at least one additional component. In some embodiments, the substantially solid silk composition is present in the mixture at about 0.01 wt. % to about 10.0 wt. % relative to the total weight of the mixture. In some embodiments, the substantially solid silk composition is present in the mixture at about 0.01 wt. % to about 1.0 wt. % relative to the total weight of the mixture. In some embodiments, the substantially solid silk composition is present in the mixture at about 1.0 wt. % to about 2.0 wt. % relative to the total weight of the mixture. In some embodiments, the substantially solid silk composition is present in the mixture at about 2.0 wt. % to about 3.0 wt. % relative to the total weight of the mixture. In some embodiments, the substantially solid silk composition is present in the mixture at about 3.0 wt. % to about 4.0 wt. % relative to the total weight of the mixture. In some embodiments, the substantially solid silk composition is present in the mixture at about 4.0 wt. % to about 5.0 wt. % relative to the total weight of the mixture. In some embodiments, the substantially solid silk composition is present in the mixture at about 5.0 wt. % to about 6.0 wt. % relative to the total weight of the mixture. In some embodiments, the mixture is a personal care composition formulated as an oral care composition, a skin care composition, a hair care composition, a cosmetic composition, a makeup composition, a sun care composition, a deodorant, an antiperspirant composition, a nail cosmetic composition, a skin cleansing composition, an aromatic cosmetic, or a bath cosmetic composition. In some embodiments, the additional component is selected from the group consisting of a filler, a diluent, a remineralizing agent, an anti-calculus agent, an anti-plaque agent, a buffer, an abrasive, an alkali metal bicarbonate salt, a binder, a thickening agent, a humectant, a whitening agent, a bleaching agent, a stain removing agent, a surfactant, titanium dioxide, a flavoring agent, xylitol, a coloring agent, a foaming agent, a sweetener, an antibacterial agent, a preservative, a vitamin, a pH adjusting agent, an anti-caries agent, a desensitizing agent, a coolant, a salivating agent, a warming agent, a numbing agent, a chelating agent, and combinations thereof. In some embodiments, the additional component is selected from the group consisting of fluoride, calcium source compound, phosphate source compound, calcium carbonate, sodium hydrogen phosphate, sodium dihydrogen phosphate, potassium hydrogen phosphate, potassium dihydrogen phosphate, amorphous calcium phosphate (ACP), tricalcium phosphate, casein phosphoprotein-ACP, bioactive glass, calcium sodium phosphosilicate, arginine bicarbonate-calcium carbonate complex, and combinations thereof. In some embodiments, the additional component is selected from the group consisting of a film-forming agent, a suspending agent, a thixotropic agent, a coloring agent, a pigment, a glitter, a plasticizer, a thickening agent, a nail hydrating agent, a nail hardening agent, boric acid, a vitamin, a plant extract, an essential oil, a preservative, a mineral salt, a fruit extract, an algae extract, a fungus extract, a caviar extract, a vegetable oil, an amino acid, a peptide, a protein, a ceramide, allantoin or an allantoin derivative, an organosilicon derivative, an antioxidant, a UV light absorber, a moisturizer, a stabilizer, a fragrance, a micronutrient, a solvent, and combinations thereof. In some embodiments, the additional component is selected from the group consisting of butanediol, propanediol, ethanediol, glycerol, butantetraol, xylitol, D-sorbitol, inositol, polyethylene glycol, hydroxyethyl cellulose, hydroxypropyl methylcellulose, dextran, gelatin, carboxymethyl cellulose, propylene glycol, polysorbate 80, polyvinyl alcohol, povidone, saponin, sucrose, fructose, maltose, carrageenan, chitosan, alginate, hyaluronic acid, and combinations thereof. In some embodiments, the additional component is selected from the group consisting of methanol, ethanol, propanol, isopropanol, acetonitrile, and combinations thereof. In some embodiments, the additional component is selected from the group consisting of hydrocarbon oil, mineral oil, silicon oil, fatty acid having 8 to 32 carbon atoms, fatty alcohol having 8 to 32 carbon atoms, synthetic ester oil derived from the esterification product of fatty acid having 8 to 32 carbon atoms and an alcohol, fatty acid glyceride, glyceryl trioctanoate, glyceryl triisopalmitate, cholesteryl isostearate, isopropyl palmitate, isopropyl myristate, neopentyl glycol dicaprate, isopropyl isostearate, octadecyl myristate, cetyl 2-ethylhexanoate, cetearyl isononanoate, cetearyl octanoate, isononyl isononanoate, isotridecyl isononanoate, glyceryl tri-2-ethylhexanoate, glyceryl tri(caprylatelcaprate), diethylene glycol monoethyl ether oleate, dicaprylyl ether, caprylic acid/capric acid propylene glycol diester, isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexylate, dipentaerythritol fatty acid ester, N-alkyl glycol monoisostearate, neopentyl glycol dicaprate, diisostearyl malate, glyceryl di-2-heptylundecanoate, trimethylolpropane tri-2-ethylhexylate, trimethylolpropane triisostearate, pentaneerythritol tetra-2-ethylhexylate, glyceryl tri-2-ethylhexylate, trimethylolpropane triisostearate, cetyl 2-ethylhexanoate, 2-ethylhexyl palmitate, glyceryl trimyristate, tri-2-heptylundecanoic glyceride, oleyl oleate, cetostearyl alcohol, 2-heptylundecyl palmitate, diisopropyl adipate, N-lauroyl-L-glutamic acid-2-octyldodecyl ester, di-2-heptylundecyl adipate, ethyl laurate, di-2-ethylhexyl cebatate. 2-hexyldecyl myristate, 2-hexyldecyl palmitate, 2-hexyldecyl adipate, diisopropyl cebatate, 2-ethylhexyl succinate, ethyl acetate, butyl acetate, amyl acetate and triethyl citrate. In some embodiments, the additional component is selected from the group consisting of liquid fat, solid fat, avocado oil, tsubaki oil, turtle oil, macadamia nut oil, corn oil, mink oil, olive oil, rape seed oil, egg yolk oil, sesame seed oil, persic oil, wheat germ oil, sasanqua oil, castor oil, linseed oil, safflower oil, cotton seed oil, perilla oil, soybean oil, peanut oil, tea seed oil, kaya oil, rice bran oil, Chinese wood oil, Japanese wood oil, jojoba oil, germ oil, sweet almond oil, rosehip seed oil, calendula oil, grape seed oil, apricot kernel oil, flaxseed oil, hazelnut oil, walnut oil, pecan nut oil, sesame oil, emu oil, coconut oil, sunflower oil, canola oil, algae oil, cacao butter, horse tallow, hardened coconut oil, palm oil, beef tallow, sheep tallow, pork tallow, hardened beef tallow, palm kernel oil, Japanese core wax, hydrogenated castor oil, and combinations thereof. In some embodiments, the additional component is selected from the group consisting of butter, petrolatum, polyethylene wax, polypropylene wax, Japanese wax, beeswax, candelilla wax, paraffin wax, ozokerite, microcrystalline wax, carnauba wax, cotton wax, esparto wax, bayberry wax, tree wax, whale wax, montan wax, bran wax, lanolin wax, kapok wax, lanolin acetate, sugar cane wax, lanolin fatty acid isopropyl ester, hexyl laurate, reduced lanolin, jojoba wax, hard lanolin, shellac wax, POE lanolin alcohol ether, lanolin alcohols with 40 moles ethylene oxide, lanolin alcohols with 65-70 moles ethylene oxide, POE lanolin alcohol acetate, POE cholesterol ether, lanolin fatty acid, POE hydrogenated lanolin alcohol ether, and combinations thereof. In some embodiments, the additional component is selected from the group consisting of phospholipid, polymer-lipid conjugate, carbohydrate-lipid conjugate, dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-dimyristoyl-sn-glycero-3-phosphorylglycerol (DMPG); 1,2-distearoyl-sn-glycero-3-phosphoethanolamine (DSPE); 1,2-dioleoyl-sn-glycero-3-phosphocholine (DOPC); 1,2-dioleoyl-sn-glycero-3-phosphoethanolamine (DOPE); 1,2-dipalmitoyl-sn-glycero-3-phospho-(1′-rac-glycerol) (DPPG); 1,2-distearoyl-snglycero-3-phosphocholine (DSPC), distearoylphosphoethanolamine conjugated with polyethylene glycol (DSPE-PEG); phosphatidylserine (PS), phosphatidylethanolamine (PE), phosphatidylglycerol (PG), phosphatidylcholine (PC), cholesterol, 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-diarachidyl-sn-glycero-3-phosphoethanolamine (20:0 PE), 1-stearoyl-2-linoleoyl-sn-glycero-3-phosphoethanolamine, 1,2-diheptadecanoyl-sn-glycero-3-phosphocholine (17:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

In some embodiments, the additional component is selected from the group consisting of hydroxypropyl methylcellulose, an acrylic polymer, a vinyl chloride copolymer, a vinyl acetate copolymer, an olefin polymer, an olefin copolymer, polyethylene, polypropylene, polystyrene, polyvinyl alcohol, an ethylene-vinyl acetate copolymer, an ethylene-vinyl alcohol copolymer, a diene polymer, a diene copolymer, polybutadiene, an ethylene propylene diene monomers (EPDM) rubber, a styrene-butadiene copolymer, a butadiene acrylonitrile rubber, a polyamide, polyamide-6, polyamide-66, a polyester, polyethylene terephthalate, a hydrocarbon polymer, a polyolefin, and polypropylene.

In an embodiment, this disclosure provides a silk oral care composition comprising silk fibroin fragments having an average weight average molecular weight selected from between about 5 kDa to about 10 kDa, from between about 6 kDa to about 17 kDa, from between about 10 kDa to about 15 kDa, from between about 15 kDa to about 20 kDa, from between about 17 kDa to about 39 kDa, from between about 20 kDa to about 25 kDa, from between about 25 kDa to about 30 kDa, from between about 30 kDa to about 35 kDa, from between about 35 kDa to about 40 kDa, from between about 39 kDa to about 80 kDa, from between about 40 kDa to about 45 kDa, from between about 45 kDa to about 50 kDa, from between about 60 kDa to about 100 kDa, and from between about 80 kDa to about 144 kDa, a polydispersity ranging from 1 to about 5; from 0 to 500 ppm lithium bromide; from 0 to 500 ppm sodium carbonate; a dental care active agent; and one or more dentally acceptable excipients. In some embodiments, the silk fibroin fragments have a polydispersity ranging from 1.0 to about 1.5. In some embodiments, the silk fibroin fragments have a polydispersity ranging from about 1.5 to about 2.0. In some embodiments, the silk fibroin fragments have a polydispersity ranging from about 1.5 to about 3.0. In some embodiments, the silk fibroin fragments have a polydispersity ranging from about 2.0 to about 2.5. In some embodiments, the silk fibroin fragments have a polydispersity ranging from about 2.5 to about 3.0. In some embodiments, the silk fibroin fragments have a polydispersity ranging from about 3.0 to about 3.5. In some embodiments, the silk fibroin fragments have a polydispersity ranging from about 3.5 to about 4.0. In some embodiments, the silk fibroin fragments have a polydispersity ranging from about 4.0 to about 4.5. In some embodiments, the silk fibroin fragments have a polydispersity ranging from about 4.5 to about 5.0. In some embodiments, the silk fibroin fragments are present at an amount ranging from about 0.01 wt. % to about 10.0 wt. % by the total weight of the silk oral care composition. In some embodiments, the silk fibroin fragments are present at an amount ranging from about 0.01 wt. % to about 1.0 wt. % by the total weight of the silk oral care composition. In some embodiments, the silk fibroin fragments are present at an amount ranging from about 1.0 wt. % to about 2.0 wt. % by the total weight of the silk oral care composition. In some embodiments, the silk fibroin fragments are present at an amount ranging from about 2.0 wt. % to about 3.0 wt. % by the total weight of the silk oral care composition. In some embodiments, the silk fibroin fragments are present at an amount ranging from about 3.0 wt. % to about 4.0 wt. % by the total weight of the silk oral care composition. In some embodiments, the silk fibroin fragments are present at an amount ranging from about 4.0 wt. % to about 5.0 wt. % by the total weight of the silk oral care composition. In some embodiments, the silk fibroin fragments are present at an amount ranging from about 5.0 wt. % to about 6.0 wt. % by the total weight of the silk oral care composition. In some embodiments, the silk oral care composition further comprises about 0.01% (w/w) to about 10% (w/w) sericin by the total weight of the silk oral care composition. In some embodiments, the silk fibroin fragments in the silk oral care composition do not spontaneously or gradually gelate and do not visibly change in color or turbidity when in an aqueous solution for at least 10 days prior to be formulated into the silk oral care composition.

In some embodiments, the composition is formulated as a solution. In some embodiments, the composition is formulated as an emulsion. In some embodiments, the composition is formulated as a powder. In some embodiments, the composition is formulated as a plurality of granules. In some embodiments, the composition is formulated as a gel. In some embodiments, the composition is formulated as a film. In some embodiments, the composition is formulated as a suspension. In some embodiments, the active agent is selected from the group consisting of therapeutic agent, plaque removal agent, germicidal agent, anticalculus agents, abrasive polishing agent, whitening/bleaching/stain removing agent, anti-plaque agent, anti-tartar agents, anti-caries agents, remineralizing agent, humectant, and combinations thereof. In some embodiments, the remineralizing agent is selected from the group consisting of fluoride, calcium source compound, phosphate source compound, calcium carbonate, sodium hydrogen phosphate, sodium dihydrogen phosphate, potassium hydrogen phosphate, potassium dihydrogen phosphate, amorphous calcium phosphate (ACP), tricalcium phosphate, casein phosphoprotein-ACP, bioactive glass, calcium sodium phosphosilicate, arginine bicarbonate-calcium carbonate complex, and combinations thereof. In some embodiments, the therapeutic agent is selected from the group consisting of fluoride salt (sodium fluoride, stannous fluoride, sodium monofluorophosphate, ammonium fluoride), strontium salt, potassium salt, stannous fluoride, phosphate fluoride, hydrogen peroxide, potassium chlorate, potassium permanganates, clove oil, wintergreen, pontacaine, hemostatic agent, zinc salt, antioxidant, antibiotic, antimicrobials, antiseptic agent, antifungal agent, anesthetic agent, antiviral agent, anti-ulcer active agent, anti-allergic agent, anti-analgesic agent, analgesic, hemostatic agent, anti-inflammatory agent (flubiprofen, naproxen, ketoprofen, aspirin), growth factor, anti-tumor agent, desensitizing agent, hormones, Vitamin, amino acid, vaccine, caffeine, monoclonal antibody, enzyme, and combinations thereof. In some embodiments, the zinc salt is selected from the group consisting of Zinc chloride, Zinc acetate, Zinc phenol, Sulfonate, Zinc borate, Zinc bromide, Zinc nitrate, Zinc glycerophosphate, Zinc benzoate, Zinc carbonate, Zinc citrate, Zinc hexafluorosilicate, Zinc diacetate trihydrate, Zinc oxide, Zinc peroxide, Zinc Salicylate, Zinc silicate, Zinc Stannate, Zinc tannate, Zinc titanate, Zinc tetrafluoroborate, Zinc gluconate, and Zinc glycinate. In some embodiments, the a desensitizing agent is one or more strontium salts selected from the group consisting of strontium chloride, strontium bromide, strontium iodide, strontium acetate, strontium edetate, strontium nitrate, strontium salicylate, strontium lactate, and combinations thereof. In some embodiments, the antioxidant is selected from the group consisting of vitamin A, vitamin E, pyruvate B-carotene, selenium, N-acetylcysteine, vitamin C, superoxide dismutase (SOD), catalase, glutathione peroxidase, glutathione reductase, and combinations thereof.

In some embodiments, the disclosure provides a silk oral care article comprising a silk oral care composition as described herein and a support. In some embodiments, the support comprises a pellet, wood stick, metal stick, paper, a yarn, a thread, a fiber, a fabric layer, a film, and a hydrogel. In some embodiments, the fabric layer comprises one or more of a natural fiber or yarn comprising one or more of cotton and wool, or a synthetic fiber or yarn comprising one or more of polyester, nylon, polyester-polyurethane copolymer, polyamide, polyaramid, polytetrafluoroethylene, polyethylene, polypropylene, polyurethane, silicone, polyurethane, polyethyleneglycol, polypropylene (PP), thermoplastic polyurethane (TPU), polyethylene (PE), Nylon and combinations thereof. In some embodiments, the fabric layer comprises a nonwoven portion. In some embodiments, the nonwoven portion comprises one or more of cellulose, cotton, rayon, regenerated cellulose, chitosan, silk, polypropylene (PP), thermoplastic polyurethane (TPU), polyethylene (PE), Nylon and combinations thereof. In some embodiments, the silk oral care composition is formulated into a product selected from the group consisting of a dental sheath, a dental patch, a floss, a tooth powder, a tooth tablet, capsule, lozenge, pastille, a toothpick, a whitening strip, a confectionary, a chewing gum, a tooth brushing sheet, toothpaste bite, an impregnated implement, a mouth piece, and an oral care strip. In some embodiments, the article is selected from a dental sheath, a dental patch, a floss, a tooth powder, a tooth tablet, capsule, lozenge, pastille, a toothpick, a whitening strip, a confectionary, a chewing gum, a tooth brushing sheet, toothpaste bite, an impregnated implement, a mouth piece, and an oral care strip

### BRIEF DESCRIPTION OF THE DRAWINGS

FIGS. 1A-B illustrate the emulsification efficiency of the 1% w/v of medium molecular weight silk fibroin protein fragments on oils having low polarity index (i.e., jojoba oil) as compared with those of 1% w/v sorbitan laurate (Span 20). Sp. 3, Sp. 4, Sp. 5 and Sp. 6 of FIG. 1A illustrated 1% w/v silk fibroin protein fragment emulsified jojoba oil at increasing volume ratio of oil to water at 0.2, 0.4, 0.6, 0.8 in a water/oil system. Sp. 7, Sp. 8, Sp. 9 and Sp. 10 of FIG. 1B illustrated 1% w/v sorbitan laurate emulsified jojoba oil at increasing volume ratio of oil to water at 0.2, 0.4, 0.6, 0.8 in a water/oil system.

FIG. 2 illustrate the creaming index evaluation results for Examples Sp. 3, Sp. 4, Sp. 5 and Sp. 6, Sp. 7, Sp. 8, Sp. 9 and Sp. 10 using the oil/water composition without surfactant as negative control.

FIG. 3. Illustrates an 80% w/v jojoba oil emulsion stabilized by Low-MW silk fibroin protein fragments having medium molecular weight at a concentration ranging from 0.6% w/v (Sp. 12), 1.2% w/v (Sp. 13) and 2.4% w/v (Sp. 14).

FIG. 4. Illustrates an 80% squalane emulsion stabilized by Low-MW silk fibroin protein fragments having low molecular weight at a concentration ranging from 0.6% w/v (Sp. 15), 1.2% w/v (Sp. 16) and 2.4% w/v (Sp. 17).

FIG. 5 illustrates the emulsion stability test for Sp. 12, Sp. 16 and control emulsion without surfactant as measured by oil separation after subjecting the silk fibroin protein fragment stabilized emulsions to various stirring conditions at 500 rpm, 900 rpm, 1000 rpm, 1500 rpm.

FIG. 6 illustrates the emulsion stability test for Sp. 12, Sp. 13, Sp. 15, Sp. 16 and control emulsion without surfactant as measured by oil separation after subjecting the silk fibroin protein fragment stabilized emulsions to various stirring conditions at 500 rpm, 900 rpm, 1000 rpm, 1500 rpm.

FIGS. 7A-E illustrate the foam tests for various surfactant systems for foams formed by shaking the surfactant water mixture for 10 second. Then the foams formed thereof were allowed to stand for a period including t=0 minute, t=5 minutes, t=15 minutes, t=30 minutes, and t=45 minutes. At each of these time points, the volume of the remained foam are evaluated. The surfactant systems studied include 6% w/v glucoside (Sp. 16), 6% w/v rhamnolipid (Sp. 17), 1% w/v glucoside and 5% w/v silk fibroin protein (Sp. 18), and 6% sophorolipid (Sp. 19).

FIG. 8 is the diagram for the measured surface tension of a silk-glucoside surfactant system in which the total surfactant concentration was fixed at 6% w/v. The glucoside concentration was varied from 0.3% w/v to 5.5% w/v, the silk fibroin fragments concentration was adjusted such as the total concentration to remain 6.0% w/v.

FIG. 9 is the diagram for the measured surface tension of a silk-glucoside surfactant system in which the total surfactant concentration was fixed at 6% w/v. The silk fibroin fragments concentration was varied from 0.3% w/v to 5.5% w/v, the glucoside was adjusted such as the total concentration to remain 6.0% w/v and pH at 5.5.

FIG. 10 illustrates the surface tension reduction at the air-water interface of 6% w/v surfactant solution by various surfactants including CAPB (cocamidopropyl betaine), sophorolipid, SLES (sodium laureth sulfate), rhamnolipid, surfactant blend of 14:2 SLES:CAPB, glucoside, surfactant blend of 0.5% glucoside and 5.5% silk fibroin protein. Cocamidopropyl betaine (CAPB) is a mixture of closely related organic compounds derived from coconut oil and dimethylaminopropylamine.

FIGS. 11A-E illustrate the effects of thickeners on the stability of foam stabilized by the surfactant system 5.5% w/v silk fibroin protein and 0.5% w/v for foams formed by shaking 20 mL of the above surfactant water mixture for 10 second. Then the foams formed thereof were allowed to stand for a period including t=0 minute, t=5 minutes, t=15 minutes, t=30 minutes, and t=45 minutes. At each of these time points, the volume of the remained foam are evaluated. In Tube A, the thickener added is 0.025 g of carrageenan (0.125% w/v). In tube B, the thickener added is 0.025 g of xanthan gum (0.125% w/v). In tube C, no thickener is added and the sample is a negative control.

FIG. 12 illustrates the effects of thickeners on the surface tension reduction at the air-water interface of a surfactant solution containing 5.5% w/v silk fibroin protein and 0.5% w/v glucoside by xanthan gum and carrageenan in varying amount ranging from 0 g, 0.1 g, 0.15 g, 0.2 g and 0.25 g. The surfactant solution containing 5.5% w/v silk fibroin protein and 0.5% w/v glucoside has a surface tension of 26.4816 mN/m. The surfactant solution with added carrageenan gave a slightly lower surface tension than did with xanthan gum.

FIG. 13A illustrates the effects of shear rate on the viscosities of a surfactant solution containing 5.5% w/v silk fibroin protein, 0.5% w/v glucoside and xanthan gum in varying amount ranging from 0.1 g, 0.15 g and 0.2 g. All viscosities were measured at 25° C. FIG. 13B illustrates the effects of concentration of thickener on the viscosity measured at shear rate of 11/s for xanthan gum in varying amount ranging from 0 g, 0.1 g, 0.15 g, 0.2 g and 0.25 g. Pure xanthan gum at 0.1 g in water has a viscosity of 1.77 Pa·s. FIG. 13C illustrates the effects of shear rate and concentration of thickener on the viscosity measured without shear for carrageenan in varying amount ranging from 0.025 g, 0.05 g, 0.1 g, 0.15 g, 0.2 g, 0.3 g, 0.35 g, 0.4 g and 0.45 g. FIG. 13D illustrates the comparison of effects of carrageen and xanthan gum on viscosity of the surfactant solution containing 5.5% w/v silk fibroin protein and 0.5% w/v. glucoside.

FIG. 14 illustrates the comparison of the surface tension testing results for the silk protein and SLES at the air-water interface.

FIG. 15 illustrates the surface tension testing results for the silk protein in combination with SLES and CAPB.

FIG. 16 illustrates the surface tension testing results for the silk protein in combination with sophorolipid and rhamnolipid.

FIG. 17A illustrates the effects of 0.1 g (0.5 wt. %) of carrageenan (CG) on the flow sweep of the 20 mL aqueous solution containing 5.5% w/v silk fibroin (SF) and 0.5% w/v. caprylyl/capryl glucoside (CCG); SF:CCG (11:1).

FIG. 17B illustrates the effects of 0.1 g (0.5 wt. %) of xanthan gum (XG) on the flow Sweep of 5.5 wt % silk fibroin (SF) and 0.5 wt % caprylyl/capryl glucoside (CCG); SF:CCG (11:1).

FIG. 18A illustrates the effects of 0.1 g (0.5 wt. %) of carrageenan (CG) on the storage and loss modulus of the 20 mL aqueous solution containing 5.5% w/v silk fibroin (SF) and 0.5% w/v. caprylyl/capryl glucoside (CCG); SF:CCG (11:1).

FIG. 18B illustrates the effects of 0.1 g (0.5 wt. %) of xanthan gum (XG) on the storage and loss modulus of the 20 mL aqueous solution containing 5.5% w/v silk fibroin (SF) and 0.5% w/v. caprylyl/capryl glucoside (CCG); SF:CCG (11:1).

FIG. 19 illustrates the effects of different amounts of carrageenan and xanthan gum on the surface tension of the 20 mL aqueous solution containing 5.5% w/v silk fibroin protein and 0.5% w/v. glucoside.

FIGS. 20A-C illustrate Low-MW silk solid resulted from lyophilization (Example 2a below) at different stages of grinding. FIG. 20A illustrate the coarse particles of the Low-MW silk solid immediate after removal from the lyophilization bottle. FIG. 20B illustrates the reduced size particle midway through grinding. FIG. 20C illustrates the fine particles with even size distribution at the completion grinding.

FIG. 21 illustrates solid particles of Mid-MW silk solid.

FIG. 22 illustrates example of two different particle size solid silk particles formed during thin film evaporation in Example 8b described herein.

FIGS. 23A and 23B illustrate examples of microparticles prepared from solution precipitation process in Example 8c described herein.

FIG. 24 illustrates that silk fibroin can adopt different conformations. Schematic representation of silk fibroin heavy chain in unordered (center), stacked beta-sheet (left) and macromolecular micelle (right) conformations. Micellar structure formation is facilitated by the hydrophobic/hydrophilic block copolymer nature of silk.

FIG. 25 illustrates a graph of experimental data demonstrating surface tension of commonly used surfactants and SF. All samples were prepared at 6 wt. % concentration.

FIG. 26 illustrates a graph of experimental data demonstrating the evaluation of synergism between SF and CCG on surface tension. Surface tension of various SF:CCG mixtures. All mixtures have a fixed total surfactant concentration (6 wt. %). As the soluble silk fibroin concentration increases (and glucoside concentration decreases) the surface tension is reduced. The lowest surface tension observed for the system is significantly lower than the one of either surfactant alone (44.58 mN/m for soluble silk fibroin and 28.21 mN/m for glucoside) suggesting a synergistic effect.

FIG. 27 illustrates a graph of experimental data demonstrating that the SF/CCG co-surfactant system at a ratio of 11:1 (SF:CCG) displays lower surface tension than other commercial surfactants.

FIG. 28A-FIG. 28E illustrate foamability and foam stability test results. In each panel, from left to right: CCG, rhamnolipid (RhL), SF:CCG (5:1) and sophorolipid (SoL) solutions (6 wt. %) are shown above from left to right respectively. The time of the photo been taken were as follows: FIG. 28A—Post-shaking, 0 min; FIG. 28B—After 5 min; FIG. 28C—After 15 min; FIG. 28D—After 30 min; FIG. 28E—After 45 min.

FIG. 29 illustrates a graph of experimental data demonstrating results from sebum removal tests for different surfactant systems. Synergistic enhancement of sebum removal is observed when SF and CCG are combined in comparison to its individual components.

FIG. 30A-FIG. 30E illustrate a foamability test of SF/CCG (11:1) with and without rheology modifiers. Samples contain only SF/CCG (right vials) or 0.125% of either carrageenan or xanthan gums (left and middle vials). Pictures taken at: FIG. 30A—0 min; FIG. 30B—5 min; FIG. 30C—min; FIG. 30D—30 min; FIG. 30E—45 min.

FIG. 31 illustrates a schematic representation of the proposed integration of capryl glucoside hydrophobic (yellow tails) and hydrophilic (red circles) domains into soluble silk fibroin micelle with hydrophilic domains (blue) and hydrophobic domains (yellow).

FIG. 32 is a flow chart showing various embodiments for producing silk fibroin protein fragments (SPFs) of the present disclosure.

FIG. 33 is a flow chart showing various parameters that can be modified during the process of producing a silk protein fragment solution of the present disclosure during the extraction and the dissolution steps.

### DETAILED DESCRIPTION

Silk is a natural polymer produced by a variety of insects and spiders. Silk produced by Bombyx mori (silkworm) comprises a filament core protein, silk fibroin, and a glue-like coating consisting of a nonfilamentous protein, sericin. Silk fibroin is a FDA approved, edible, non-toxic, and relative inexpensive silkworm cocoon derived proteins. The structure and content of amino acids in silk fibroin protein are very similar to the skin of the human body.

Methods of making silk fibroin or silk fibroin fragments are known and are described for example in U.S. Pat. Nos. 9,187,538, 9,511,012, 9,517,191, 9,522,107, 9,522,108, 9,545,369, and 10,166,177, all of which are incorporated herein in their entireties. Methods of using sericin in skin care compositions are known as protective film, moisturizer, antioxidant, liquid cleaning emulsion on skin/hair and are described for example in U.S. Patent Publications Nos. 6,497,893, and 5,415,813.

Silk fibroin proteins have found applications in personal care products. In these disclosures, silk fibroin protein fragment solutions were used because of the low solubility of the raw silk fibroin proteins. While providing some beneficial coating effect, the silk fibroin peptides are not as effective as the intact proteins. The silk fibroin powder was reported as an additive in skin care products formulated as soap bar, face creams, toilet powders, face powders, skin barrier compositions etc. (U.S. Pat. Nos. 2,194,858, 4,233,212, 6,497,893). However, this silk fibroin protein powder is insoluble in water and is not as effective in film forming and coating for skin care applications as a water-soluble silk protein. The natural or recombinant spider silk proteins were reported as active ingredient for incorporation into cosmetic and dermatological compositions such as hair care, skin care, make-up, and sunscreen products (U.S. Pat. No. 6,280,747). However, the spider silk is not water-soluble. Therefore, the beneficial effects of the self-assembly and coating properties of the spider silk proteins are not realized.

Silk fibroin protein has shown enormous potentials in various fields, however, application of silk fibroin in emulsion technology is rather limited (See Wen et al., ACS Omega, 2018, vol. 3, pp. 3396-3405). Further, there are very few reports on surfactant system containing silk fibroin and conventional surfactants.

Throughout the cosmetic industry, the desire for surfactants to be biodegradable and biocompatible is almost as desirable as efficient performance. This desire has led to the implementation of biosurfactants and sugar surfactants within the industry. Biosurfactants are synthesize by micro-organisms and reduce interfacial and surface tension similar to chemical surfactants. Besides the obvious advantages of biosurfactants to chemical surfactants like higher biodegradability, superior environmental compatibility, and decreased toxicity amounts, biosurfactants also have higher foaming abilities and lower critical micelle concentration. These advantages demonstrate that biosurfactants have many advantageous characterizes for application in the cosmetic industry as well as health, chemical, petroleum, food and agricultural industries.

The disclosure provides silk fibroin protein fragments as emulsifier, surfactant to stabilize personal care compositions for the topical delivery of personal care active agents.

In an embodiment, this disclosure provides a silk personal care composition comprising (i) silk fibroin protein fragments that are substantially devoid of sericin at weight percent ranging from about 0.0001 wt. % to about 10.0 wt. % by the total weight of the silk personal care composition, (ii) at least one personal care active agent, and (iii) a carrier, wherein the silk fibroin-based protein fragments have a weight average molecular weight selected from between about 5 kDa to about 144 kDa, wherein the silk fibroin-based protein fragments have a polydispersity of between about 1.5 and about 3.0.

In some embodiments, the silk personal care composition is formulate for topical application and in a form selected from the group consisting of an aqueous solution, an ethanolic solution, an oil, a gel, a foam, an emulsion, a suspension, a mousses, a solid (e.g., wax), a film, a lozenge, an oral tablet, a solid, a lotion, a cream, an aerosol spray, a paste, a stick, a fabric, a mesh, a sponge, powder, an ointment, a liniment, a balm, a spray and a tonic.

In some embodiments, the silk personal care composition is a personal care product selected from the group consisting of a feminine hygiene product, a beauty soap, a soap bar, a facial wash, a hand wash, a body wash, a cleansing wipe, a cleansing pad, a cleansing foam, a rinse, a cleansing lotion, a cleansing milk, a cleansing gel, a cleansing soap bar, an exfoliating product, a bath and shower soap in bar, a cream, an emulsion, a shaving or after-shave cream, a foam, a conditioner, a cologne, a shaving or after-shave lotion, a hair care product, a shampoo, a hair conditioner, a hair spray, a perfume, a cosmetic oil, a facial mask, a moisturizer, an anti-wrinkle cream, an anti-wrinkle lotion, an eye lotion, an eye cream, a tanning cream, a tanning lotion, a tanning emulsion, a sunscreen cream, a sunscreen lotion, a sunscreen emulsion, a tanning oil, a sunscreen oil, a hand lotion, a body lotion, a color cosmetic, a mascara, a lipstick, a lip liner, an eye shadow, an eye-liner, a rouge, a face powder, a foundation, a blush, a perfume, a bath soap in bar, a bath product, a toothpaste, a dentifrice, a tooth powder, an oral gel, an aqueous gel, a non-aqueous gel, a mouth rinse, a mouth spray, a plaque removing liquid, a denture product, a dental solution, a lozenge, an oral tablet, a chewing gum, a candy, a fast-dissolving film, a strip, a dental floss, a tooth glossing product, a finishing product, an impregnated dental implement, a remineralizing gel, a remineralizing mouthwash, a remineralizing tooth powder, a remineralizing chewing gums, a remineralizing lozenge, a remineralizing toothpaste, a antiperspirant stick, a roll-on deodorant, a powder deodorant, a gel deodorant, an aerosol deodorant, a paste deodorant, a nail polish, and a nail polish remover.

In some embodiments, the silk personal care composition is transparent, or translucent.

In some embodiments, the silk personal care product containing silk fibroin protein fragments as described above, at least one natural surfactant (e.g. glucoside, sucrose ester), at least one personal care active agent and at least one rheology modifier, e.g., a xanthan gum.

In some embodiments, the silk personal care product contains at most 13 different composition ingredients. In some embodiments, the silk personal care product contains less than twelve different composition ingredients.

### Definitions

As used in the preceding sections and throughout the rest of this specification, unless defined otherwise, all technical and scientific terms used herein have the same meaning as is commonly understood by one skilled in the art to which this disclosure belongs. All patents and publications referred to herein are incorporated by reference in their entireties.

All percentages, parts and ratios are based upon the total weight of the collagen boosting compositions of the present disclosure, unless otherwise specified. All such weights as they pertain to listed ingredients are based on the active level and, therefore, do not include solvents or by-products that may be included in commercially available materials, unless otherwise specified. The term “weight percent” may be denoted as “wt. %” or % w/w herein.

As used herein, the term “a”, “an”, or “the” generally is construed to cover both the singular and the plural forms.

As used herein, the term “about” generally refers to a particular numeric value that is within an acceptable error range as determined by one of ordinary skill in the art, which will depend in part on how the numeric value is measured or determined, i.e., the limitations of the measurement system. For example, “about” can mean a range of ±20%, ±10%, or ±5% of a given numeric value.

As used herein, the term “dermatologically acceptable carrier” means a carrier suitable for use in contact with mammalian keratinous tissue without causing any adverse effects such as undue toxicity, incompatibility, instability, allergic response, for example. A dermatologically acceptable carrier may include, without limitations, water, liquid or solid emollients, humectants, solvents, and the like.

As used herein, the term “hydrophilic-lipophilic balance” (HLB) of a surfactant is a measure of the degree to which it is hydrophilic or hydrophobic, as determined by calculating values for the different regions of the molecule, as described by Griffin's method HLB=20*Mₕ/M, where Mₕ is the molecular mass of the hydrophilic portion of the surfactant, and M is the molecular mass of the entire surfactant molecule, giving a result on a scale of 0 to 20. A HLB value of 0 corresponds to a completely lipophilic molecule, and a value of 20 corresponds to a completely hydrophilic molecule. The HLB value can be used to predict the surfactant properties of a molecule: HLB<10: Lipid-soluble (water-insoluble), HLB>10: Water-soluble (lipid-insoluble), HLB=1-3: anti-foaming agent, 3-6: W/O (water-in-oil) emulsifier, 7-9: wetting and spreading agent, 8-16: O/W (oil-in-water) emulsifier, 13-16: detergent, 16-18: solubilizer or hydrotrope.

As used herein, “average weight average molecular weight” refers to an average of two or more values of weight average molecular weight of silk fibroin or fragments thereof of the same compositions, the two or more values determined by two or more separate experimental readings.

As used herein, the term polymer “polydispersity (PD)” is generally used as a measure of the broadness of a molecular weight distribution of a polymer, and is defined by the formula polydispersity PD=Mw/Mn.

As used herein, the term “substantially homogeneous” may refer to silk fibroin-based protein fragments that are distributed in a normal distribution about an identified molecular weight. As used herein, the term “substantially homogeneous” may refer to an even distribution of a component or an additive, for example, silk fibroin fragments, dermatologically acceptable carrier, etc., throughout a composition of the present disclosure.

As used herein, the terms “silk fibroin peptide,” “silk fibroin protein fragment,” and “silk fibroin fragment” are used interchangeably. Molecular weight or number of amino acids units are defined when molecular size becomes an important parameter.

As used herein, the term “cosmetic benefit” refers to a desired cosmetic change that results from the administration of the silk personal care composition. Cosmetic benefits include but are not limited to improvements in the condition of skin, hair, nail and the oral cavity. In preferred embodiments, at least one cosmetic benefit is provided by the skin care, oral care, hair care, nail care and makeup compositions of the present disclosure.

As used herein, the term “cosmetically acceptable” refers to approved by a regulatory agency of the appropriate governmental agency or listed in the U.S. Pharmacopoeia or other generally recognized pharmacopoeia for use in animals, and more particularly in humans.

As used herein, the term “dentifrice” refers to pastes, gels, or liquid formulations unless otherwise specified. The dentifrice composition may be a single-phase composition or may be a combination of two or more dentifrice compositions. The dentifrice composition may be in any desired form, such as deep striped, surface striped, multilayered, having the gel surrounding the paste, or any combination thereof. Each dentifrice composition in a dentifrice comprising two or more separate dentifrice compositions may be contained in a physically separated compartment of a dispenser and dispensed side-by-side.

As used herein, the term “detergent” refers to a substance or preparation containing soaps and/or other surfactants intended for washing and cleaning processes. Thus, detergents are cleansing agents that differ from soap but can also emulsify oils and hold dirt in suspension. Detergents may be in any form (liquid, powder, paste, bar, cake, molded piece, etc.) and used e.g., in personal care products.

As used herein, the term “film former” or “film forming agent” refers to a polymer or resin that leaves a film on the substrate to which it is applied.

As used herein, the term “long wear” compositions refers to compositions where skin care active agent (e.g., color produced by lipid stick) remains the same or substantially the same as at the time of application, as viewed by the naked eye, after an extended period of time.

As used herein, the term “lozenge” refers to breath mints, troches, pastilles, microcapsules, and fast-dissolving solid forms including freeze dried forms (cakes, wafers, thin films), and compressed tablets.

As used herein, the term “makeup compositions” refer to cosmetic preparations that are used to beautify, caring for, maintaining, or augment the appearance of a human or other animal. “Makeup compositions” include, but are not limited to color cosmetics, mascaras, lipsticks, lip liners, eye shadows, eyeliners, rouges, face powders, foundations, blushes, and nail polish.

As used herein, the term “mild” refers to the silk fibroin fragments based compositions and products thereof demonstrate skin mildness comparable to a mild alkyl glyceryl ether sulfonate surfactant based soap bar.

As used herein, the term “nail care composition” refers to compositions that are applied to the nails to provide beneficial properties such as harder and stronger nails, nail color, etc.

As used herein, the term “nonwoven sheet” refers to a sheet having a structure of individual fibers or threads which are interlaid, but not in an identifiable manner as in a knitted fabric. Nonwoven sheets or fabrics have been formed from many processes, such as, meltblowing processes, spunbonding processes, and bonded carded web processes. The basis weight of nonwoven fabrics is usually expressed in ounces of material per square yard (osy) or grams per square meter (gsm) and the fibers diameters are usually expressed in microns. (Note that to convert from osy to gsm, multiply osy by 33.91).

As used herein, the term “meltblown fiber” refers to fibers formed by extruding a molten thermoplastic material through a plurality of fine, usually circular, die capillaries as molten threads or filaments into converging high velocity, usually hot, gas (e.g. air) streams which attenuate the filaments of thermoplastic material to reduce their diameter, which may be to microfiber diameter. The meltblown fibers are generally tacky when deposited on a collecting surface.

As used herein, “spunbond fibers” refers to small diameter fibers which are formed by extruding molten thermoplastic material as filaments from a plurality of fine, usually circular capillaries of a spinneret with the diameter of the extruded filaments then being rapidly reduced. The spunbond fibers are generally not tacky when they are deposited on a collecting surface.

As used herein, the term “oral care composition” refers to a product which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. The oral care composition of the present disclosure may be in the form of a toothpaste, dentifrice, tooth powder, topical oral gel, mouth rinse, denture product, mouth spray, lozenge, oral tablet, or chewing gum.

As used herein, the term “oral care product” is defined as a product which can be used for maintaining and/or improving oral hygiene in the mouth of humans and animals, and/or preventing or treating dental diseases, tooth whitening. Oral care product can have any suitable physical form (i.e. powder, paste, gel, liquid, ointment, tablet etc.).

As used herein, the terms “peptide” or “protein” refers to a chain of amino acids that are held together by peptide bonds (also called amide bonds). The basic distinguishing factors for proteins and peptides are size and structure. Peptides are smaller than proteins. Traditionally, peptides are defined as molecules that consist of between 2 and 50 amino acids, whereas proteins are made up of 50 or more amino acids. In addition, peptides tend to be less well defined in structure than proteins, which can adopt complex conformations known as secondary, tertiary, and quaternary structures.

As used herein, the term “remineralization” refers to a natural process in which a tooth's minerals are restored or replaced. Remineralization reverses the process of decay and/or erosion caused from demineralization.

As used herein, the term “skin care composition” refers to compositions that are applied to skin in order to provide beneficial properties, including, but not limited to, wrinkle minimizing, wrinkle removal, decoloring, coloring, skin softening, skin smoothing, depilation, cleansing, relubricating dry skin, compensating the loss of lipid and water caused by daily washing, delay skin aging etc. In some embodiments, this disclosure provides skin care compositions that improve skin tone. In these embodiments, the skin tone improvement comprises lessening of wrinkles, smoothing skin texture, moisturizing skin, and other desired cosmetic benefits.

As used herein, the term “fibroin” or “silk protein” is a type of structural protein produced by certain spider and insect species that produce silk (See definition provided in WIPO Pearl-WIPO's Multilingual Terminology Portal database, https://wipopearl.wipo.int/en/linguistic). Fibroin may include silkworm fibroin, insect or spider silk protein (e.g., spidroin), recombinant spider protein, silk proteins present in other spider silk types, e.g., tubuliform silk protein (TuSP), flagelliform silk protein, minor ampullate silk proteins, aciniform silk protein, pyriform silk protein, aggregate silk glue), silkworm fibroin produced by genetically modified silkworm, or recombinant silkworm fibroin.

As used herein, the term “silk fibroin” refers to silkworm fibroin, silk fibroin produced by genetically modified silkworm, or recombinant silkworm fibroin (See (1) Narayan Ed., Encyclopedia of Biomedical Engineering, Vol. 2, Elsevier, 2019; (2) Kobayashi et al. Eds, Encyclopedia of Polymeric Nanomaterials, Springer, 2014, https://link.springer.com/referenceworkentry/10.1007%2F978-3-642-36199-9_323-1). In an embodiment, silk fibroin is obtained from Bombyx mori.

As used herein, the term “substantially homogeneous” may refer to silk fibroin protein fragments that are distributed in a normal distribution about an identified molecular weight. As used herein, the term “substantially homogeneous” may also refer to an even distribution of a component or an additive, for example, silk fibroin fragments, dermatologically acceptable carrier, etc., throughout the silk personal care composition.

As used herein, the term “surface tension” refers to the tendency of fluid surfaces to shrink into the minimum surface area possible. At liquid-air interfaces, surface tension results from the greater attraction of liquid molecules to each other (due to cohesion) than to the molecules in the air (due to adhesion). The net effect is an inward force at its surface that causes the liquid to behave as if its surface were covered with a stretched elastic membrane. Because of the relatively high attraction of water molecules to each other through a web of hydrogen bonds, water has a higher surface tension (72.8 mN/m at 20° C.) than most other liquids.

As used herein, the term “transfer resistance” refers to the quality exhibited by compositions that are not readily removed by contact with another material, such as, for example, an item of clothing. Transfer resistance may be evaluated by any method known in the art for evaluating such. For example, transfer resistance of a composition may be evaluated by a modified “kiss” test. The modified “kiss” test may involve application of the composition to a fingernail followed by rubbing a material, for example, a sheet of paper, against the nail after expiration of a certain amount of time following application, such as 5 minutes after application. Similarly, transfer resistance of a composition may be evaluated by the amount of product transferred from a wearer to any other substrate, such as transfer from the nail of an individual to a sleeve when putting on clothing after the expiration of a certain amount of time following application of the composition to the nail. The amount of composition transferred to the substrate (e.g., sleeve or paper) may then be evaluated and compared. For example, a nail polish composition may be transfer resistant if a majority of the product is left on the wearer's nails. Further, the amount transferred may be compared with that transferred by other compositions, such as commercially available compositions.

### SPF Definitions and Properties

As used herein, “silk protein fragments” (SPF) include, without limitation, one or more of: “silk fibroin fragments” as defined herein; “recombinant silk fragments” as defined herein; “spider silk fragments” as defined herein; “silk fibroin-like protein fragments” as defined herein; “chemically modified silk fragments” as defined herein; and/or “sericin or sericin fragments” as defined herein. SPF may have any molecular weight values or ranges described herein, and any polydispersity values or ranges described herein. As used herein, in some embodiments the term “silk protein fragment” also refers to a silk protein that comprises or consists of at least two identical repetitive units which each independently selected from naturally-occurring silk polypeptides or of variations thereof, amino acid sequences of naturally-occurring silk polypeptides, or of combinations of both.

### SPF Molecular Weight and Polydispersity

In an embodiment, a composition of the present disclosure includes SPF having an average weight average molecular weight selected from between about 1 to about 5 kDa. In an embodiment, a composition of the present disclosure includes SPF having an average weight average molecular weight selected from between about 5 to about 10 kDa. In an embodiment, a composition of the present disclosure includes SPF having an average weight average molecular weight selected from between about 10 to about 15 kDa. In an embodiment, a composition of the present disclosure includes SPF having an average weight average molecular weight selected from between about 15 to about 20 kDa. In an embodiment, a composition of the present disclosure includes SPF having an average weight average molecular weight selected from between about 14 to about 30 kDa. In an embodiment, a composition of the present disclosure includes SPF having an average weight average molecular weight selected from between about 20 to about 25 kDa. In an embodiment, a composition of the present disclosure includes SPF having an average weight average molecular weight selected from between about 25 to about 30 kDa. In an embodiment, a composition of the present disclosure includes SPF having an average weight average molecular weight selected from between about 30 to about 35 kDa. In an embodiment, a composition of the present disclosure includes SPF having an average weight average molecular weight selected from between about 35 to about 40 kDa. In an embodiment, a composition of the present disclosure includes SPF having an average weight average molecular weight selected from between about 39 to about 54 kDa. In an embodiment, a composition of the present disclosure includes SPF having an average weight average molecular weight selected from between about 40 to about 45 kDa. In an embodiment, a composition of the present disclosure includes SPF having an average weight average molecular weight selected from between about 45 to about 50 kDa. In an embodiment, a composition of the present disclosure includes SPF having an average weight average molecular weight selected from between about 50 to about 55 kDa. In an embodiment, a composition of the present disclosure includes SPF having an average weight average molecular weight selected from between about 55 to about 60 kDa. In an embodiment, a composition of the present disclosure includes SPF having an average weight average molecular weight selected from between about 60 to about 65 kDa. In an embodiment, a composition of the present disclosure includes SPF having an average weight average molecular weight selected from between about 65 to about 70 kDa. In an embodiment, a composition of the present disclosure includes SPF having an average weight average molecular weight selected from between about 70 to about 75 kDa. In an embodiment, a composition of the present disclosure includes SPF having an average weight average molecular weight selected from between about 75 to about 80 kDa. In an embodiment, a composition of the present disclosure includes SPF having an average weight average molecular weight selected from between about 80 to about 85 kDa. In an embodiment, a composition of the present disclosure includes SPF having an average weight average molecular weight selected from between about 85 to about 90 kDa. In an embodiment, a composition of the present disclosure includes SPF having an average weight average molecular weight selected from between about 90 to about 95 kDa. In an embodiment, a composition of the present disclosure includes SPF having an average weight average molecular weight selected from between about 95 to about 100 kDa. In an embodiment, a composition of the present disclosure includes SPF having an average weight average molecular weight selected from between about 100 to about 105 kDa. In an embodiment, a composition of the present disclosure includes SPF having an average weight average molecular weight selected from between about 105 to about 110 kDa. In an embodiment, a composition of the present disclosure includes SPF having an average weight average molecular weight selected from between about 110 to about 115 kDa. In an embodiment, a composition of the present disclosure includes SPF having an average weight average molecular weight selected from between about 115 to about 120 kDa. In an embodiment, a composition of the present disclosure includes SPF having an average weight average molecular weight selected from between about 120 to about 125 kDa. In an embodiment, a composition of the present disclosure includes SPF having an average weight average molecular weight selected from between about 125 to about 130 kDa. In an embodiment, a composition of the present disclosure includes SPF having an average weight average molecular weight selected from between about 130 to about 135 kDa. In an embodiment, a composition of the present disclosure includes SPF having an average weight average molecular weight selected from between about 135 to about 140 kDa. In an embodiment, a composition of the present disclosure includes SPF having an average weight average molecular weight selected from between about 140 to about 145 kDa. In an embodiment, a composition of the present disclosure includes SPF having an average weight average molecular weight selected from between about 145 to about 150 kDa. In an embodiment, a composition of the present disclosure includes SPF having an average weight average molecular weight selected from between about 150 to about 155 kDa. In an embodiment, a composition of the present disclosure includes SPF having an average weight average molecular weight selected from between about 155 to about 160 kDa. In an embodiment, a composition of the present disclosure includes SPF having an average weight average molecular weight selected from between about 160 to about 165 kDa. In an embodiment, a composition of the present disclosure includes SPF having an average weight average molecular weight selected from between about 165 to about 170 kDa. In an embodiment, a composition of the present disclosure includes SPF having an average weight average molecular weight selected from between about 170 to about 175 kDa. In an embodiment, a composition of the present disclosure includes SPF having an average weight average molecular weight selected from between about 175 to about 180 kDa. In an embodiment, a composition of the present disclosure includes SPF having an average weight average molecular weight selected from between about 180 to about 185 kDa. In an embodiment, a composition of the present disclosure includes SPF having an average weight average molecular weight selected from between about 185 to about 190 kDa. In an embodiment, a composition of the present disclosure includes SPF having an average weight average molecular weight selected from between about 190 to about 195 kDa. In an embodiment, a composition of the present disclosure includes SPF having an average weight average molecular weight selected from between about 195 to about 200 kDa. In an embodiment, a composition of the present disclosure includes SPF having an average weight average molecular weight selected from between about 200 to about 205 kDa. In an embodiment, a composition of the present disclosure includes SPF having an average weight average molecular weight selected from between about 205 to about 210 kDa. In an embodiment, a composition of the present disclosure includes SPF having an average weight average molecular weight selected from between about 210 to about 215 kDa. In an embodiment, a composition of the present disclosure includes SPF having an average weight average molecular weight selected from between about 215 to about 220 kDa. In an embodiment, a composition of the present disclosure includes SPF having an average weight average molecular weight selected from between about 220 to about 225 kDa. In an embodiment, a composition of the present disclosure includes SPF having an average weight average molecular weight selected from between about 225 to about 230 kDa. In an embodiment, a composition of the present disclosure includes SPF having an average weight average molecular weight selected from between about 230 to about 235 kDa. In an embodiment, a composition of the present disclosure includes SPF having an average weight average molecular weight selected from between about 235 to about 240 kDa. In an embodiment, a composition of the present disclosure includes SPF having an average weight average molecular weight selected from between about 240 to about 245 kDa. In an embodiment, a composition of the present disclosure includes SPF having an average weight average molecular weight selected from between about 245 to about 250 kDa. In an embodiment, a composition of the present disclosure includes SPF having an average weight average molecular weight selected from between about 250 to about 255 kDa. In an embodiment, a composition of the present disclosure includes SPF having an average weight average molecular weight selected from between about 255 to about 260 kDa. In an embodiment, a composition of the present disclosure includes SPF having an average weight average molecular weight selected from between about 260 to about 265 kDa. In an embodiment, a composition of the present disclosure includes SPF having an average weight average molecular weight selected from between about 265 to about 270 kDa. In an embodiment, a composition of the present disclosure includes SPF having an average weight average molecular weight selected from between about 270 to about 275 kDa. In an embodiment, a composition of the present disclosure includes SPF having an average weight average molecular weight selected from between about 275 to about 280 kDa. In an embodiment, a composition of the present disclosure includes SPF having an average weight average molecular weight selected from between about 280 to about 285 kDa. In an embodiment, a composition of the present disclosure includes SPF having an average weight average molecular weight selected from between about 285 to about 290 kDa. In an embodiment, a composition of the present disclosure includes SPF having an average weight average molecular weight selected from between about 290 to about 295 kDa. In an embodiment, a composition of the present disclosure includes SPF having an average weight average molecular weight selected from between about 295 to about 300 kDa. In an embodiment, a composition of the present disclosure includes SPF having an average weight average molecular weight selected from between about 300 to about 305 kDa. In an embodiment, a composition of the present disclosure includes SPF having an average weight average molecular weight selected from between about 305 to about 310 kDa. In an embodiment, a composition of the present disclosure includes SPF having an average weight average molecular weight selected from between about 310 to about 315 kDa. In an embodiment, a composition of the present disclosure includes SPF having an average weight average molecular weight selected from between about 315 to about 320 kDa. In an embodiment, a composition of the present disclosure includes SPF having an average weight average molecular weight selected from between about 320 to about 325 kDa. In an embodiment, a composition of the present disclosure includes SPF having an average weight average molecular weight selected from between about 325 to about 330 kDa. In an embodiment, a composition of the present disclosure includes SPF having an average weight average molecular weight selected from between about 330 to about 335 kDa. In an embodiment, a composition of the present disclosure includes SPF having an average weight average molecular weight selected from between about 335 to about 340 kDa. In an embodiment, a composition of the present disclosure includes SPF having an average weight average molecular weight selected from between about 340 to about 345 kDa. In an embodiment, a composition of the present disclosure includes SPF having an average weight average molecular weight selected from between about 345 to about 350 kDa.

In some embodiments, compositions of the present disclosure include SPF compositions selected from compositions #1001 to #2450, having weight average molecular weights selected from about 1 kDa to about 145 kDa, and a polydispersity selected from between 1 and about 5 (including, without limitation, a polydispersity of 1), between 1 and about 1.5 (including, without limitation, a polydispersity of 1), between about 1.5 and about 2, between about 1.5 and about 3, between about 2 and about 2.5, between about 2.5 and about 3, between about 3 and about 3.5, between about 3.5 and about 4, between about 4 and about 4.5, and between about 4.5 and about 5:

| MW | PDI (about) | | | | | | | | | |
|---|---|---|---|---|---|---|---|---|---|---|
| (about) | 1-5 | 1-1.5 | 1.5-2 | 1.5-3 | 2-2.5 | 2.5-3 | 3-3.5 | 3.5-4 | 4-4.5 | 4.5-5 |
| 1 kDa | 1001 | 1002 | 1003 | 1004 | 1005 | 1006 | 1007 | 1008 | 1009 | 1010 |
| 2 kDa | 1011 | 1012 | 1013 | 1014 | 1015 | 1016 | 1017 | 1018 | 1019 | 1020 |
| 3 kDa | 1021 | 1022 | 1023 | 1024 | 1025 | 1026 | 1027 | 1028 | 1029 | 1030 |
| 4 kDa | 1031 | 1032 | 1033 | 1034 | 1035 | 1036 | 1037 | 1038 | 1039 | 1040 |
| 5 kDa | 1041 | 1042 | 1043 | 1044 | 1045 | 1046 | 1047 | 1048 | 1049 | 1050 |
| 6 kDa | 1051 | 1052 | 1053 | 1054 | 1055 | 1056 | 1057 | 1058 | 1059 | 1060 |
| 7 kDa | 1061 | 1062 | 1063 | 1064 | 1065 | 1066 | 1067 | 1068 | 1069 | 1070 |
| 8 kDa | 1071 | 1072 | 1073 | 1074 | 1075 | 1076 | 1077 | 1078 | 1079 | 1080 |
| 9 kDa | 1081 | 1082 | 1083 | 1084 | 1085 | 1086 | 1087 | 1088 | 1089 | 1090 |
| 10 kDa | 1091 | 1092 | 1093 | 1094 | 1095 | 1096 | 1097 | 1098 | 1099 | 1100 |
| 11 kDa | 1101 | 1102 | 1103 | 1104 | 1105 | 1106 | 1107 | 1108 | 1109 | 1110 |
| 12 kDa | 1111 | 1112 | 1113 | 1114 | 1115 | 1116 | 1117 | 1118 | 1119 | 1120 |
| 13 kDa | 1121 | 1122 | 1123 | 1124 | 1125 | 1126 | 1127 | 1128 | 1129 | 1130 |
| 14 kDa | 1131 | 1132 | 1133 | 1134 | 1135 | 1136 | 1137 | 1138 | 1139 | 1140 |
| 15 kDa | 1141 | 1142 | 1143 | 1144 | 1145 | 1146 | 1147 | 1148 | 1149 | 1150 |
| 16 kDa | 1151 | 1152 | 1153 | 1154 | 1155 | 1156 | 1157 | 1158 | 1159 | 1160 |
| 17 kDa | 1161 | 1162 | 1163 | 1164 | 1165 | 1166 | 1167 | 1168 | 1169 | 1170 |
| 18 kDa | 1171 | 1172 | 1173 | 1174 | 1175 | 1176 | 1177 | 1178 | 1179 | 1180 |
| 19 kDa | 1181 | 1182 | 1183 | 1184 | 1185 | 1186 | 1187 | 1188 | 1189 | 1190 |
| 20 kDa | 1191 | 1192 | 1193 | 1194 | 1195 | 1196 | 1197 | 1198 | 1199 | 1200 |
| 21 kDa | 1201 | 1202 | 1203 | 1204 | 1205 | 1206 | 1207 | 1208 | 1209 | 1210 |
| 22 kDa | 1211 | 1212 | 1213 | 1214 | 1215 | 1216 | 1217 | 1218 | 1219 | 1220 |
| 23 kDa | 1221 | 1222 | 1223 | 1224 | 1225 | 1226 | 1227 | 1228 | 1229 | 1230 |
| 24 kDa | 1231 | 1232 | 1233 | 1234 | 1235 | 1236 | 1237 | 1238 | 1239 | 1240 |
| 25 kDa | 1241 | 1242 | 1243 | 1244 | 1245 | 1246 | 1247 | 1248 | 1249 | 1250 |
| 26 kDa | 1251 | 1252 | 1253 | 1254 | 1255 | 1256 | 1257 | 1258 | 1259 | 1260 |
| 27 kDa | 1261 | 1262 | 1263 | 1264 | 1265 | 1266 | 1267 | 1268 | 1269 | 1270 |
| 28 kDa | 1271 | 1272 | 1273 | 1274 | 1275 | 1276 | 1277 | 1278 | 1279 | 1280 |
| 29 kDa | 1281 | 1282 | 1283 | 1284 | 1285 | 1286 | 1287 | 1288 | 1289 | 1290 |
| 30 kDa | 1291 | 1292 | 1293 | 1294 | 1295 | 1296 | 1297 | 1298 | 1299 | 1300 |
| 31 kDa | 1301 | 1302 | 1303 | 1304 | 1305 | 1306 | 1307 | 1308 | 1309 | 1310 |
| 32 kDa | 1311 | 1312 | 1313 | 1314 | 1315 | 1316 | 1317 | 1318 | 1319 | 1320 |
| 33 kDa | 1321 | 1322 | 1323 | 1324 | 1325 | 1326 | 1327 | 1328 | 1329 | 1330 |
| 34 kDa | 1331 | 1332 | 1333 | 1334 | 1335 | 1336 | 1337 | 1338 | 1339 | 1340 |
| 35 kDa | 1341 | 1342 | 1343 | 1344 | 1345 | 1346 | 1347 | 1348 | 1349 | 1350 |
| 36 kDa | 1351 | 1352 | 1353 | 1354 | 1355 | 1356 | 1357 | 1358 | 1359 | 1360 |
| 37 kDa | 1361 | 1362 | 1363 | 1364 | 1365 | 1366 | 1367 | 1368 | 1369 | 1370 |
| 38 kDa | 1371 | 1372 | 1373 | 1374 | 1375 | 1376 | 1377 | 1378 | 1379 | 1380 |
| 39 kDa | 1381 | 1382 | 1383 | 1384 | 1385 | 1386 | 1387 | 1388 | 1389 | 1390 |
| 40 kDa | 1391 | 1392 | 1393 | 1394 | 1395 | 1396 | 1397 | 1398 | 1399 | 1400 |
| 41 kDa | 1401 | 1402 | 1403 | 1404 | 1405 | 1406 | 1407 | 1408 | 1409 | 1410 |
| 42 kDa | 1411 | 1412 | 1413 | 1414 | 1415 | 1416 | 1417 | 1418 | 1419 | 1420 |
| 43 kDa | 1421 | 1422 | 1423 | 1424 | 1425 | 1426 | 1427 | 1428 | 1429 | 1430 |
| 44 kDa | 1431 | 1432 | 1433 | 1434 | 1435 | 1436 | 1437 | 1438 | 1439 | 1440 |
| 45 kDa | 1441 | 1442 | 1443 | 1444 | 1445 | 1446 | 1447 | 1448 | 1449 | 1450 |
| 46 kDa | 1451 | 1452 | 1453 | 1454 | 1455 | 1456 | 1457 | 1458 | 1459 | 1460 |
| 47 kDa | 1461 | 1462 | 1463 | 1464 | 1465 | 1466 | 1467 | 1468 | 1469 | 1470 |
| 48 kDa | 1471 | 1472 | 1473 | 1474 | 1475 | 1476 | 1477 | 1478 | 1479 | 1480 |
| 49 kDa | 1481 | 1482 | 1483 | 1484 | 1485 | 1486 | 1487 | 1488 | 1489 | 1490 |
| 50 kDa | 1491 | 1492 | 1493 | 1494 | 1495 | 1496 | 1497 | 1498 | 1499 | 1500 |
| 51 kDa | 1501 | 1502 | 1503 | 1504 | 1505 | 1506 | 1507 | 1508 | 1509 | 1510 |
| 52 kDa | 1511 | 1512 | 1513 | 1514 | 1515 | 1516 | 1517 | 1518 | 1519 | 1520 |
| 53 kDa | 1521 | 1522 | 1523 | 1524 | 1525 | 1526 | 1527 | 1528 | 1529 | 1530 |
| 54 kDa | 1531 | 1532 | 1533 | 1534 | 1535 | 1536 | 1537 | 1538 | 1539 | 1540 |
| 55 kDa | 1541 | 1542 | 1543 | 1544 | 1545 | 1546 | 1547 | 1548 | 1549 | 1550 |
| 56 kDa | 1551 | 1552 | 1553 | 1554 | 1555 | 1556 | 1557 | 1558 | 1559 | 1560 |
| 57 kDa | 1561 | 1562 | 1563 | 1564 | 1565 | 1566 | 1567 | 1568 | 1569 | 1570 |
| 58 kDa | 1571 | 1572 | 1573 | 1574 | 1575 | 1576 | 1577 | 1578 | 1579 | 1580 |
| 59 kDa | 1581 | 1582 | 1583 | 1584 | 1585 | 1586 | 1587 | 1588 | 1589 | 1590 |
| 60 kDa | 1591 | 1592 | 1593 | 1594 | 1595 | 1596 | 1597 | 1598 | 1599 | 1600 |
| 61 kDa | 1601 | 1602 | 1603 | 1604 | 1605 | 1606 | 1607 | 1608 | 1609 | 1610 |
| 62 kDa | 1611 | 1612 | 1613 | 1614 | 1615 | 1616 | 1617 | 1618 | 1619 | 1620 |
| 63 kDa | 1621 | 1622 | 1623 | 1624 | 1625 | 1626 | 1627 | 1628 | 1629 | 1630 |
| 64 kDa | 1631 | 1632 | 1633 | 1634 | 1635 | 1636 | 1637 | 1638 | 1639 | 1640 |
| 65 kDa | 1641 | 1642 | 1643 | 1644 | 1645 | 1646 | 1647 | 1648 | 1649 | 1650 |
| 66 kDa | 1651 | 1652 | 1653 | 1654 | 1655 | 1656 | 1657 | 1658 | 1659 | 1660 |
| 67 kDa | 1661 | 1662 | 1663 | 1664 | 1665 | 1666 | 1667 | 1668 | 1669 | 1670 |
| 68 kDa | 1671 | 1672 | 1673 | 1674 | 1675 | 1676 | 1677 | 1678 | 1679 | 1680 |
| 69 kDa | 1681 | 1682 | 1683 | 1684 | 1685 | 1686 | 1687 | 1688 | 1689 | 1690 |
| 70 kDa | 1691 | 1692 | 1693 | 1694 | 1695 | 1696 | 1697 | 1698 | 1699 | 1700 |
| 71 kDa | 1701 | 1702 | 1703 | 1704 | 1705 | 1706 | 1707 | 1708 | 1709 | 1710 |
| 72 kDa | 1711 | 1712 | 1713 | 1714 | 1715 | 1716 | 1717 | 1718 | 1719 | 1720 |
| 73 kDa | 1721 | 1722 | 1723 | 1724 | 1725 | 1726 | 1727 | 1728 | 1729 | 1730 |
| 74 kDa | 1731 | 1732 | 1733 | 1734 | 1735 | 1736 | 1737 | 1738 | 1739 | 1740 |
| 75 kDa | 1741 | 1742 | 1743 | 1744 | 1745 | 1746 | 1747 | 1748 | 1749 | 1750 |
| 76 kDa | 1751 | 1752 | 1753 | 1754 | 1755 | 1756 | 1757 | 1758 | 1759 | 1760 |
| 77 kDa | 1761 | 1762 | 1763 | 1764 | 1765 | 1766 | 1767 | 1768 | 1769 | 1770 |
| 78 kDa | 1771 | 1772 | 1773 | 1774 | 1775 | 1776 | 1777 | 1778 | 1779 | 1780 |
| 79 kDa | 1781 | 1782 | 1783 | 1784 | 1785 | 1786 | 1787 | 1788 | 1789 | 1790 |
| 80 kDa | 1791 | 1792 | 1793 | 1794 | 1795 | 1796 | 1797 | 1798 | 1799 | 1800 |
| 81 kDa | 1801 | 1802 | 1803 | 1804 | 1805 | 1806 | 1807 | 1808 | 1809 | 1810 |
| 82 kDa | 1811 | 1812 | 1813 | 1814 | 1815 | 1816 | 1817 | 1818 | 1819 | 1820 |
| 83 kDa | 1821 | 1822 | 1823 | 1824 | 1825 | 1826 | 1827 | 1828 | 1829 | 1830 |
| 84 kDa | 1831 | 1832 | 1833 | 1834 | 1835 | 1836 | 1837 | 1838 | 1839 | 1840 |
| 85 kDa | 1841 | 1842 | 1843 | 1844 | 1845 | 1846 | 1847 | 1848 | 1849 | 1850 |
| 86 kDa | 1851 | 1852 | 1853 | 1854 | 1855 | 1856 | 1857 | 1858 | 1859 | 1860 |
| 87 kDa | 1861 | 1862 | 1863 | 1864 | 1865 | 1866 | 1867 | 1868 | 1869 | 1870 |
| 88 kDa | 1871 | 1872 | 1873 | 1874 | 1875 | 1876 | 1877 | 1878 | 1879 | 1880 |
| 89 kDa | 1881 | 1882 | 1883 | 1884 | 1885 | 1886 | 1887 | 1888 | 1889 | 1890 |
| 90 kDa | 1891 | 1892 | 1893 | 1894 | 1895 | 1896 | 1897 | 1898 | 1899 | 1900 |
| 91 kDa | 1901 | 1902 | 1903 | 1904 | 1905 | 1906 | 1907 | 1908 | 1909 | 1910 |
| 92 kDa | 1911 | 1912 | 1913 | 1914 | 1915 | 1916 | 1917 | 1918 | 1919 | 1920 |
| 93 kDa | 1921 | 1922 | 1923 | 1924 | 1925 | 1926 | 1927 | 1928 | 1929 | 1930 |
| 94 kDa | 1931 | 1932 | 1933 | 1934 | 1935 | 1936 | 1937 | 1938 | 1939 | 1940 |
| 95 kDa | 1941 | 1942 | 1943 | 1944 | 1945 | 1946 | 1947 | 1948 | 1949 | 1950 |
| 96 kDa | 1951 | 1952 | 1953 | 1954 | 1955 | 1956 | 1957 | 1958 | 1959 | 1960 |
| 97 kDa | 1961 | 1962 | 1963 | 1964 | 1965 | 1966 | 1967 | 1968 | 1969 | 1970 |
| 98 kDa | 1971 | 1972 | 1973 | 1974 | 1975 | 1976 | 1977 | 1978 | 1979 | 1980 |
| 99 kDa | 1981 | 1982 | 1983 | 1984 | 1985 | 1986 | 1987 | 1988 | 1989 | 1990 |
| 100 kDa | 1991 | 1992 | 1993 | 1994 | 1995 | 1996 | 1997 | 1998 | 1999 | 2000 |
| 101 kDa | 2001 | 2002 | 2003 | 2004 | 2005 | 2006 | 2007 | 2008 | 2009 | 2010 |
| 102 kDa | 2011 | 2012 | 2013 | 2014 | 2015 | 2016 | 2017 | 2018 | 2019 | 2020 |
| 103 kDa | 2021 | 2022 | 2023 | 2024 | 2025 | 2026 | 2027 | 2028 | 2029 | 2030 |
| 104 kDa | 2031 | 2032 | 2033 | 2034 | 2035 | 2036 | 2037 | 2038 | 2039 | 2040 |
| 105 kDa | 2041 | 2042 | 2043 | 2044 | 2045 | 2046 | 2047 | 2048 | 2049 | 2050 |
| 106 kDa | 2051 | 2052 | 2053 | 2054 | 2055 | 2056 | 2057 | 2058 | 2059 | 2060 |
| 107 kDa | 2061 | 2062 | 2063 | 2064 | 2065 | 2066 | 2067 | 2068 | 2069 | 2070 |
| 108 kDa | 2071 | 2072 | 2073 | 2074 | 2075 | 2076 | 2077 | 2078 | 2079 | 2080 |
| 109 kDa | 2081 | 2082 | 2083 | 2084 | 2085 | 2086 | 2087 | 2088 | 2089 | 2090 |
| 110 kDa | 2091 | 2092 | 2093 | 2094 | 2095 | 2096 | 2097 | 2098 | 2099 | 2100 |
| 111 kDa | 2101 | 2102 | 2103 | 2104 | 2105 | 2106 | 2107 | 2108 | 2109 | 2110 |
| 112 kDa | 2111 | 2112 | 2113 | 2114 | 2115 | 2116 | 2117 | 2118 | 2119 | 2120 |
| 113 kDa | 2121 | 2122 | 2123 | 2124 | 2125 | 2126 | 2127 | 2128 | 2129 | 2130 |
| 114 kDa | 2131 | 2132 | 2133 | 2134 | 2135 | 2136 | 2137 | 2138 | 2139 | 2140 |
| 115 kDa | 2141 | 2142 | 2143 | 2144 | 2145 | 2146 | 2147 | 2148 | 2149 | 2150 |
| 116 kDa | 2151 | 2152 | 2153 | 2154 | 2155 | 2156 | 2157 | 2158 | 2159 | 2160 |
| 117 kDa | 2161 | 2162 | 2163 | 2164 | 2165 | 2166 | 2167 | 2168 | 2169 | 2170 |
| 118 kDa | 2171 | 2172 | 2173 | 2174 | 2175 | 2176 | 2177 | 2178 | 2179 | 2180 |
| 119 kDa | 2181 | 2182 | 2183 | 2184 | 2185 | 2186 | 2187 | 2188 | 2189 | 2190 |
| 120 kDa | 2191 | 2192 | 2193 | 2194 | 2195 | 2196 | 2197 | 2198 | 2199 | 2200 |
| 121 kDa | 2201 | 2202 | 2203 | 2204 | 2205 | 2206 | 2207 | 2208 | 2209 | 2210 |
| 122 kDa | 2211 | 2212 | 2213 | 2214 | 2215 | 2216 | 2217 | 2218 | 2219 | 2220 |
| 123 kDa | 2221 | 2222 | 2223 | 2224 | 2225 | 2226 | 2227 | 2228 | 2229 | 2230 |
| 124 kDa | 2231 | 2232 | 2233 | 2234 | 2235 | 2236 | 2237 | 2238 | 2239 | 2240 |
| 125 kDa | 2241 | 2242 | 2243 | 2244 | 2245 | 2246 | 2247 | 2248 | 2249 | 2250 |
| 126 kDa | 2251 | 2252 | 2253 | 2254 | 2255 | 2256 | 2257 | 2258 | 2259 | 2260 |
| 127 kDa | 2261 | 2262 | 2263 | 2264 | 2265 | 2266 | 2267 | 2268 | 2269 | 2270 |
| 128 kDa | 2271 | 2272 | 2273 | 2274 | 2275 | 2276 | 2277 | 2278 | 2279 | 2280 |
| 129 kDa | 2281 | 2282 | 2283 | 2284 | 2285 | 2286 | 2287 | 2288 | 2289 | 2290 |
| 130 kDa | 2291 | 2292 | 2293 | 2294 | 2295 | 2296 | 2297 | 2298 | 2299 | 2300 |
| 131 kDa | 2301 | 2302 | 2303 | 2304 | 2305 | 2306 | 2307 | 2308 | 2309 | 2310 |
| 132 kDa | 2311 | 2312 | 2313 | 2314 | 2315 | 2316 | 2317 | 2318 | 2319 | 2320 |
| 133 kDa | 2321 | 2322 | 2323 | 2324 | 2325 | 2326 | 2327 | 2328 | 2329 | 2330 |
| 134 kDa | 2331 | 2332 | 2333 | 2334 | 2335 | 2336 | 2337 | 2338 | 2339 | 2340 |
| 135 kDa | 2341 | 2342 | 2343 | 2344 | 2345 | 2346 | 2347 | 2348 | 2349 | 2350 |
| 136 kDa | 2351 | 2352 | 2353 | 2354 | 2355 | 2356 | 2357 | 2358 | 2359 | 2360 |
| 137 kDa | 2361 | 2362 | 2363 | 2364 | 2365 | 2366 | 2367 | 2368 | 2369 | 2370 |
| 138 kDa | 2371 | 2372 | 2373 | 2374 | 2375 | 2376 | 2377 | 2378 | 2379 | 2380 |
| 139 kDa | 2381 | 2382 | 2383 | 2384 | 2385 | 2386 | 2387 | 2388 | 2389 | 2390 |
| 140 kDa | 2391 | 2392 | 2393 | 2394 | 2395 | 2396 | 2397 | 2398 | 2399 | 2400 |
| 141 kDa | 2401 | 2402 | 2403 | 2404 | 2405 | 2406 | 2407 | 2408 | 2409 | 2410 |
| 142 kDa | 2411 | 2412 | 2413 | 2414 | 2415 | 2416 | 2417 | 2418 | 2419 | 2420 |
| 143 kDa | 2421 | 2422 | 2423 | 2424 | 2425 | 2426 | 2427 | 2428 | 2429 | 2430 |
| 144 kDa | 2431 | 2432 | 2433 | 2434 | 2435 | 2436 | 2437 | 2438 | 2439 | 2440 |
| 145 kDa | 2441 | 2442 | 2443 | 2444 | 2445 | 2446 | 2447 | 2448 | 2449 | 2450 |

As used herein, “low molecular weight,” “low MW,” or “low-MW” SPF may include SPF having a weight average molecular weight, or average weight average molecular weight selected from between about 5 kDa to about 38 kDa, about 14 kDa to about 30 kDa, or about 6 kDa to about 17 kDa. In some embodiments, a target low molecular weight for certain SPF may be weight average molecular weight of about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, about 20 kDa, about 21 kDa, about 22 kDa, about 23 kDa, about 24 kDa, about 25 kDa, about 26 kDa, about 27 kDa, about 28 kDa, about 29 kDa, about 30 kDa, about 31 kDa, about 32 kDa, about 33 kDa, about 34 kDa, about 35 kDa, about 36 kDa, about 37 kDa, or about 38 kDa.

As used herein, “medium molecular weight,” “medium MW,” or “mid-MW” SPF may include SPF having a weight average molecular weight, or average weight average molecular weight selected from between about 31 kDa to about 55 kDa, or about 39 kDa to about 54 kDa. In some embodiments, a target medium molecular weight for certain SPF may be weight average molecular weight of about 31 kDa, about 32 kDa, about 33 kDa, about 34 kDa, about 35 kDa, about 36 kDa, about 37 kDa, about 38 kDa, about 39 kDa, about 40 kDa, about 41 kDa, about 42 kDa, about 43 kDa, about 44 kDa, about 45 kDa, about 46 kDa, about 47 kDa, about 48 kDa, about 49 kDa, about 50 kDa, about 51 kDa, about 52 kDa, about 53 kDa, about 54 kDa, or about 55 kDa.

As used herein, “high molecular weight,” “high MW,” or “high-MW” SPF may include SPF having a weight average molecular weight, or average weight average molecular weight selected from between about 55 kDa to about 150 kDa. In some embodiments, a target high molecular weight for certain SPF may be about 55 kDa, about 56 kDa, about 57 kDa, about 58 kDa, about 59 kDa, about 60 kDa, about 61 kDa, about 62 kDa, about 63 kDa, about 64 kDa, about 65 kDa, about 66 kDa, about 67 kDa, about 68 kDa, about 69 kDa, about 70 kDa, about 71 kDa, about 72 kDa, about 73 kDa, about 74 kDa, about 75 kDa, about 76 kDa, about 77 kDa, about 78 kDa, about 79 kDa, or about 80 kDa.

In some embodiments, the molecular weights described herein (e.g., low molecular weight silk, medium molecular weight silk, high molecular weight silk) may be converted to the approximate number of amino acids contained within the respective SPF, as would be understood by a person having ordinary skill in the art. For example, the average weight of an amino acid may be about 110 daltons (i.e., 110 g/mol). Therefore, in some embodiments, dividing the molecular weight of a linear protein by 110 daltons may be used to approximate the number of amino acid residues contained therein.

In an embodiment, SPF in a composition of the present disclosure have a polydispersity selected from between 1 to about 5.0, including, without limitation, a polydispersity of 1. In an embodiment, SPF in a composition of the present disclosure have a polydispersity selected from between about 1.5 to about 3.0. In an embodiment, SPF in a composition of the present disclosure have a polydispersity selected from between 1 to about 1.5, including, without limitation, a polydispersity of 1. In an embodiment, SPF in a composition of the present disclosure have a polydispersity selected from between about 1.5 to about 2.0. In an embodiment, SPF in a composition of the present disclosure have a polydispersity selected from between about 2.0 to about 2.5. In an embodiment, SPF in a composition of the present disclosure have a polydispersity selected from between about 2.5 to about 3.0. In an embodiment, SPF in a composition of the present disclosure have a polydispersity selected from between about 3.0 to about 3.5. In an embodiment, SPF in a composition of the present disclosure have a polydispersity selected from between about 3.5 to about 4.0. In an embodiment, SPF in a composition of the present disclosure have a polydispersity selected from between about 4.0 to about 4.5. In an embodiment, SPF in a composition of the present disclosure have a polydispersity selected from between about 4.5 to about 5.0.

In an embodiment, SPF in a composition of the present disclosure have a polydispersity of 1. In an embodiment, SPF in a composition of the present disclosure have a polydispersity of about 1.1. In an embodiment, SPF in a composition of the present disclosure have a polydispersity of about 1.2. In an embodiment, SPF in a composition of the present disclosure have a polydispersity of about 1.3. In an embodiment, SPF in a composition of the present disclosure have a polydispersity of about 1.4. In an embodiment, SPF in a composition of the present disclosure have a polydispersity of about 1.5. In an embodiment, SPF in a composition of the present disclosure have a polydispersity of about 1.6. In an embodiment, SPF in a composition of the present disclosure have a polydispersity of about 1.7. In an embodiment, SPF in a composition of the present disclosure have a polydispersity of about 1.8. In an embodiment, SPF in a composition of the present disclosure have a polydispersity of about 1.9. In an embodiment, SPF in a composition of the present disclosure have a polydispersity of about 2.0. In an embodiment, SPF in a composition of the present disclosure have a polydispersity of about 2.1. In an embodiment, SPF in a composition of the present disclosure have a polydispersity of about 2.2. In an embodiment, SPF in a composition of the present disclosure have a polydispersity of about 2.3. In an embodiment, SPF in a composition of the present disclosure have a polydispersity of about 2.4. In an embodiment, SPF in a composition of the present disclosure have a polydispersity of about 2.5. In an embodiment, SPF in a composition of the present disclosure have a polydispersity of about 2.6. In an embodiment, SPF in a composition of the present disclosure have a polydispersity of about 2.7. In an embodiment, SPF in a composition of the present disclosure have a polydispersity of about 2.8. In an embodiment, SPF in a composition of the present disclosure have a polydispersity of about 2.9. In an embodiment, SPF in a composition of the present disclosure have a polydispersity of about 3.0. In an embodiment, SPF in a composition of the present disclosure have a polydispersity of about 3.1. In an embodiment, SPF in a composition of the present disclosure have a polydispersity of about 3.2. In an embodiment, SPF in a composition of the present disclosure have a polydispersity of about 3.3. In an embodiment, SPF in a composition of the present disclosure have a polydispersity of about 3.4. In an embodiment, SPF in a composition of the present disclosure have a polydispersity of about 3.5. In an embodiment, SPF in a composition of the present disclosure have a polydispersity of about 3.6. In an embodiment, SPF in a composition of the present disclosure have a polydispersity of about 3.7. In an embodiment, SPF in a composition of the present disclosure have a polydispersity of about 3.8. In an embodiment, SPF in a composition of the present disclosure have a polydispersity of about 3.9. In an embodiment, SPF in a composition of the present disclosure have a polydispersity of about 4.0. In an embodiment, SPF in a composition of the present disclosure have a polydispersity of about 4.1. In an embodiment, SPF in a composition of the present disclosure have a polydispersity of about 4.2. In an embodiment, SPF in a composition of the present disclosure have a polydispersity of about 4.3. In an embodiment, SPF in a composition of the present disclosure have a polydispersity of about 4.4. In an embodiment, SPF in a composition of the present disclosure have a polydispersity of about 4.5. In an embodiment, SPF in a composition of the present disclosure have a polydispersity of about 4.6. In an embodiment, SPF in a composition of the present disclosure have a polydispersity of about 4.7. In an embodiment, SPF in a composition of the present disclosure have a polydispersity of about 4.8. In an embodiment, SPF in a composition of the present disclosure have a polydispersity of about 4.9. In an embodiment, SPF in a composition of the present disclosure have a polydispersity of about 5.0.

In some embodiments, in compositions described herein having combinations of low, medium, and/or high molecular weight SPF, such low, medium, and/or high molecular weight SPF may have the same or different polydispersities.

### Silk Fibroin Fragments

Methods of making silk fibroin or silk fibroin protein fragments and their applications in various fields are known and are described for example in U.S. Pat. Nos. 9,187,538, 9,511,012, 9,517,191, 9,522,107, 9,522,108, 9,545,369, and 10,166,177, 10,287,728 and 10,301,768, all of which are incorporated herein in their entireties. Raw silk from silkworm Bombyx mori is composed of two primary proteins: silk fibroin (approximately 75%) and sericin (approximately 25%). Silk fibroin is a fibrous protein with a semi-crystalline structure that provides stiffness and strength. As used herein, the term “silk fibroin” means the fibers of the cocoon of Bombyx mori having a weight average molecular weight of about 370,000 Da. The crude silkworm fiber consists of a double thread of fibroin. The adhesive substance holding these double fibers together is sericin. The silk fibroin is composed of a heavy chain having a weight average molecular weight of about 350,000 Da (H chain), and a light chain having a weight average molecular weight about 25,000 Da (L chain). Silk fibroin is an amphiphilic polymer with large hydrophobic domains occupying the major component of the polymer, which has a high molecular weight. The hydrophobic regions are interrupted by small hydrophilic spacers, and the N- and C-termini of the chains are also highly hydrophilic. The hydrophobic domains of the H-chain contain a repetitive hexapeptide sequence of Gly-Ala-Gly-Ala-Gly-Ser and repeats of Gly-Ala/Ser/Tyr dipeptides, which can form stable anti-parallel-sheet crystallites. The amino acid sequence of the L-chain is non-repetitive, so the L-chain is more hydrophilic and relatively elastic. The hydrophilic (Tyr, Ser) and hydrophobic (Gly, Ala) chain segments in silk fibroin molecules are arranged alternatively such that allows self-assembling of silk fibroin molecules.

Provided herein are methods for producing pure and highly scalable silk fibroin-protein fragment mixture solutions that may be used across multiple industries for a variety of applications. Without wishing to be bound by any particular theory, it is believed that these methods are equally applicable to fragmentation of any SPF described herein, including without limitation recombinant silk proteins, and fragmentation of silk-like or fibroin-like proteins.

As used herein, the term “fibroin” includes silk worm fibroin and insect or spider silk protein. In an embodiment, fibroin is obtained from Bombyx mori. Raw silk from Bombyx mori is composed of two primary proteins: silk fibroin (approximately 75%) and sericin (approximately 25%). Silk fibroin is a fibrous protein with a semi-crystalline structure that provides stiffness and strength. As used herein, the term “silk fibroin” means the fibers of the cocoon of Bombyx mori having a weight average molecular weight of about 370,000 Da. Conversion of these insoluble silk fibroin fibrils into water-soluble silk fibroin protein fragments requires the addition of a concentrated neutral salt (e.g., 8-10 M lithium bromide), which interferes with inter- and intramolecular ionic and hydrogen bonding that would otherwise render the fibroin protein insoluble in water. Methods of making silk fibroin protein fragments, and/or compositions thereof, are known and are described for example in U.S. Pat. Nos. 9,187,538, 9,511,012, 9,517,191, 9,522,107, 9,522,108, 9,545,369, and 10,166,177.

The raw silk cocoons from the silkworm Bombyx mori was cut into pieces. The pieces silk cocoons were processed in an aqueous solution of Na₂CO₃ at about 100° C. for about 60 minutes to remove sericin (degumming). The volume of the water used equals about 0.4× raw silk weight and the amount of Na₂CO₃ is about 0.848× the weight of the raw silk cocoon pieces. The resulting degummed silk cocoon pieces were rinsed with deionized water three times at about 60° C. (20 minutes per rinse). The volume of rinse water for each cycle was 0.2 L×the weight of the raw silk cocoon pieces. The excess water from the degummed silk cocoon pieces was removed. After the DI water washing step, the wet degummed silk cocoon pieces were dried at room temperature. The degummed silk cocoon pieces were mixed with a LiBr solution, and the mixture was heated to about 100° C. The warmed mixture was placed in a dry oven and was heated at about 100° C. for about 60 minutes to achieve complete dissolution of the native silk protein. The resulting silk fibroin solution was filtered and dialyzed using Tangential Flow Filtration (TFF) and a 10 kDa membrane against deionized water for 72 hours. The resulting silk fibroin aqueous solution has a concentration of about 8.5 wt. %. Then, 8.5% silk solution was diluted with water to result in a 1.0% w/v silk solution. TFF can then be used to further concentrate the pure silk solution to a concentration of 20.0% w/w silk to water.

Dialyzing the silk through a series of water changes is a manual and time intensive process, which could be accelerated by changing certain parameters, for example diluting the silk solution prior to dialysis. The dialysis process could be scaled for manufacturing by using semi-automated equipment, for example a tangential flow filtration system.

In some embodiments, the silk solutions are prepared under various preparation condition parameters such as: 90° C. 30 min, 90° C. 60 min, 100° C. 30 min, and 100° C. 60 min. Briefly, 9.3 M LiBr was prepared and allowed to sit at room temperature for at least 30 minutes. 5 mL of LiBr solution was added to 1.25 g of silk and placed in the 60° C. oven. Samples from each set were removed at 4, 6, 8, 12, 24, 168 and 192 hours.

In some embodiments, the silk solutions are prepared under various preparation condition parameters such as: 90° C. 30 min, 90° C. 60 min, 100° C. 30 min, and 100° C. 60 min. Briefly, 9.3 M LiBr solution was heated to one of four temperatures: 60° C., 80° C., 100° C. or boiling. 5 mL of hot LiBr solution was added to 1.25 g of silk and placed in the 60° C. oven. Samples from each set were removed at 1, 4 and 6 hours.

In some embodiments, the silk solutions are prepared under various preparation condition parameters such as: Four different silk extraction combinations were used: 90° C. 30 min, 90° C. 60 min, 100° C. 30 min, and 100° C. 60 min. Briefly, 9.3 M LiBr solution was heated to one of four temperatures: 60° C., 80° C., 100° C. or boiling. 5 mL of hot LiBr solution was added to 1.25 g of silk and placed in the oven at the same temperature of the LiBr. Samples from each set were removed at 1, 4 and 6 hours. 1 mL of each sample was added to 7.5 mL of 9.3 M LiBr and refrigerated for viscosity testing.

In some embodiments, SPF are obtained by dissolving raw unscoured, partially scoured, or scoured silkworm fibers with a neutral lithium bromide salt. The raw silkworm silks are processed under selected temperature and other conditions in order to remove any sericin and achieve the desired weight average molecular weight (Mw) and polydispersity (PD) of the fragment mixture. Selection of process parameters may be altered to achieve distinct final silk protein fragment characteristics depending upon the intended use. The resulting final fragment solution is silk fibroin protein fragments and water with parts per million (ppm) to non-detectable levels of process contaminants, levels acceptable in the pharmaceutical, medical and consumer eye care markets. The concentration, size and polydispersity of SPF may further be altered depending upon the desired use and performance requirements.

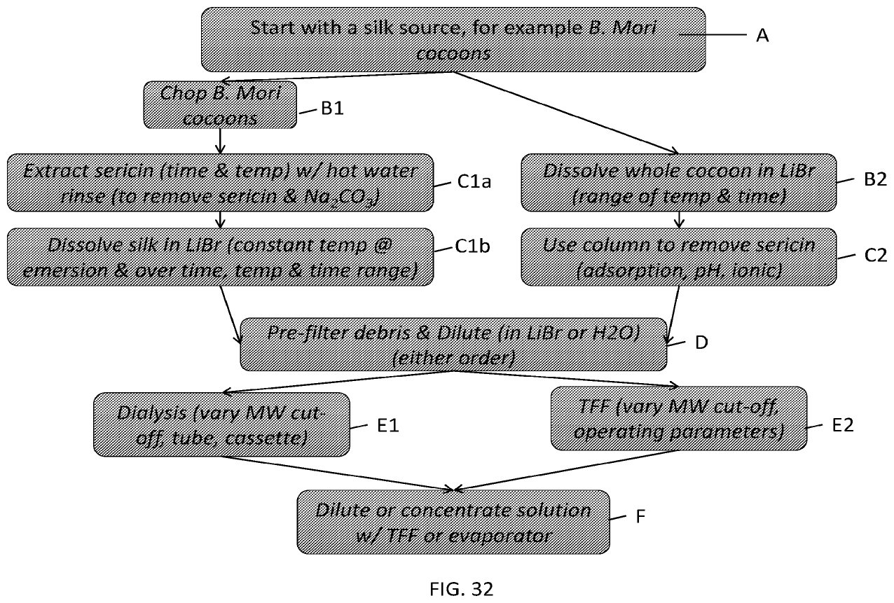

FIG. 32 is a flow chart showing various embodiments for producing pure silk fibroin protein fragments (SPFs) of the present disclosure. It should be understood that not all of the steps illustrated are necessarily required to fabricate all silk solutions of the present disclosure. As illustrated in FIG. 32, step A, cocoons (heat-treated or non-heat-treated), silk fibers, silk powder, spider silk or recombinant spider silk can be used as the silk source. If starting from raw silk cocoons from Bombyx mori, the cocoons can be cut into small pieces, for example pieces of approximately equal size, step B1. The raw silk is then extracted and rinsed to remove any sericin, step C1a. This results in substantially sericin free raw silk. In an embodiment, water is heated to a temperature between 84° C. and 100° C. (ideally boiling) and then Na₂CO₃ (sodium carbonate) is added to the boiling water until the Na₂CO₃ is completely dissolved. The raw silk is added to the boiling water/Na₂CO₃ (100° C.) and submerged for approximately 15-90 minutes, where boiling for a longer time results in smaller silk protein fragments. In an embodiment, the water volume equals about 0.4× raw silk weight and the Na₂CO₃ volume equals about 0.848× raw silk weight. In an embodiment, the water volume equals 0.1× raw silk weight and the Na₂CO₃ volume is maintained at 2.12 g/L.

Subsequently, the water dissolved Na₂CO₃ solution is drained and excess water/Na₂CO₃ is removed from the silk fibroin fibers (e.g., ring out the fibroin extract by hand, spin cycle using a machine, etc.). The resulting silk fibroin extract is rinsed with warm to hot water to remove any remaining adsorbed sericin or contaminate, typically at a temperature range of about 40° C. to about 80° C., changing the volume of water at least once (repeated for as many times as required). The resulting silk fibroin extract is a substantially sericin-depleted silk fibroin. In an embodiment, the resulting silk fibroin extract is rinsed with water at a temperature of about 60° C. In an embodiment, the volume of rinse water for each cycle equals 0.1 L to 0.2 L×raw silk weight. It may be advantageous to agitate, turn or circulate the rinse water to maximize the rinse effect. After rinsing, excess water is removed from the extracted silk fibroin fibers (e.g., ring out fibroin extract by hand or using a machine). Alternatively, methods known to one skilled in the art such as pressure, temperature, or other reagents or combinations thereof may be used for the purpose of sericin extraction. Alternatively, the silk gland (100% sericin free silk protein) can be removed directly from a worm. This would result in liquid silk protein, without any alteration of the protein structure, free of sericin.

The extracted fibroin fibers are then allowed to dry completely. Once dry, the extracted silk fibroin is dissolved using a solvent added to the silk fibroin at a temperature between ambient and boiling, step C1b. In an embodiment, the solvent is a solution of Lithium bromide (LiBr) (boiling for LiBr is 140° C.). Alternatively, the extracted fibroin fibers are not dried but wet and placed in the solvent; solvent concentration can then be varied to achieve similar concentrations as to when adding dried silk to the solvent. The final concentration of LiBr solvent can range from 0.1 M to 9.3 M. Complete dissolution of the extracted fibroin fibers can be achieved by varying the treatment time and temperature along with the concentration of dissolving solvent. Other solvents may be used including, but not limited to, phosphate phosphoric acid, calcium nitrate, calcium chloride solution or other concentrated aqueous solutions of inorganic salts. To ensure complete dissolution, the silk fibers should be fully immersed within the already heated solvent solution and then maintained at a temperature ranging from about 60° C. to about 140° C. for 1-168 hrs. In an embodiment, the silk fibers should be fully immersed within the solvent solution and then placed into a dry oven at a temperature of about 100° C. for about 1 hour.

The temperature at which the silk fibroin extract is added to the LiBr solution (or vice versa) has an effect on the time required to completely dissolve the fibroin and on the resulting molecular weight and polydispersity of the final SPF mixture solution. In an embodiment, silk solvent solution concentration is less than or equal to 20% w/v. In addition, agitation during introduction or dissolution may be used to facilitate dissolution at varying temperatures and concentrations. The temperature of the LiBr solution will provide control over the silk protein fragment mixture molecular weight and polydispersity created. In an embodiment, a higher temperature will more quickly dissolve the silk offering enhanced process scalability and mass production of silk solution. In an embodiment, using a LiBr solution heated to a temperature from 80° C. to 140° C. reduces the time required in an oven in order to achieve full dissolution. Varying time and temperature at or above 60° C. of the dissolution solvent will alter and control the MW and polydispersity of the SPF mixture solutions formed from the original molecular weight of the native silk fibroin protein.

Alternatively, whole cocoons may be placed directly into a solvent, such as LiBr, bypassing extraction, step B2. This requires subsequent filtration of silk worm particles from the silk and solvent solution and sericin removal using methods know in the art for separating hydrophobic and hydrophilic proteins such as a column separation and/or chromatography, ion exchange, chemical precipitation with salt and/or pH, and or enzymatic digestion and filtration or extraction, all methods are common examples and without limitation for standard protein separation methods, step C2. Non-heat treated cocoons with the silkworm removed, may alternatively be placed into a solvent such as LiBr, bypassing extraction. The methods described above may be used for sericin separation, with the advantage that non-heat treated cocoons will contain significantly less worm debris.

Dialysis may be used to remove the dissolution solvent from the resulting dissolved fibroin protein fragment solution by dialyzing the solution against a volume of water, step E1. Pre-filtration prior to dialysis is helpful to remove any debris (i.e., silk worm remnants) from the silk and LiBr solution, step D. In one example, a 3 μm or 5 μm filter is used with a flow-rate of 200-300 mL/min to filter a 0.1% to 1.0% silk-LiBr solution prior to dialysis and potential concentration if desired. A method disclosed herein, as described above, is to use time and/or temperature to decrease the concentration from 9.3 M LiBr to a range from 0.1 M to 9.3 M to facilitate filtration and downstream dialysis, particularly when considering creating a scalable process method. Alternatively, without the use of additional time or temperate, a 9.3 M LiBr-silk protein fragment solution may be diluted with water to facilitate debris filtration and dialysis. The result of dissolution at the desired time and temperate filtration is a translucent particle-free room temperature shelf-stable silk protein fragment-LiBr solution of a known MW and polydispersity. It is advantageous to change the dialysis water regularly until the solvent has been removed (e.g., change water after 1 hour, 4 hours, and then every 12 hours for a total of 6 water changes). The total number of water volume changes may be varied based on the resulting concentration of solvent used for silk protein dissolution and fragmentation. After dialysis, the final silk solution maybe further filtered to remove any remaining debris (i.e., silk worm remnants).

Alternatively, Tangential Flow Filtration (TFF), which is a rapid and efficient method for the separation and purification of biomolecules, may be used to remove the solvent from the resulting dissolved fibroin solution, step E2. TFF offers a highly pure aqueous silk protein fragment solution and enables scalability of the process in order to produce large volumes of the solution in a controlled and repeatable manner. The silk and LiBr solution may be diluted prior to TFF (20% down to 0.1% silk in either water or LiBr). Pre-filtration as described above prior to TFF processing may maintain filter efficiency and potentially avoids the creation of silk gel boundary layers on the filter's surface as the result of the presence of debris particles. Pre-filtration prior to TFF is also helpful to remove any remaining debris (i.e., silk worm remnants) from the silk and LiBr solution that may cause spontaneous or long-term gelation of the resulting water only solution, step D. TFF, recirculating or single pass, may be used for the creation of water-silk protein fragment solutions ranging from 0.1% silk to 30.0% silk (more preferably, 0.1%-6.0% silk). Different cutoff size TFF membranes may be required based upon the desired concentration, molecular weight and polydispersity of the silk protein fragment mixture in solution. Membranes ranging from 1-100 kDa may be necessary for varying molecular weight silk solutions created for example by varying the length of extraction boil time or the time and temperate in dissolution solvent (e.g., LiBr). In an embodiment, a TFF 5 or 10 kDa membrane is used to purify the silk protein fragment mixture solution and to create the final desired silk-to-water ratio. As well, TFF single pass, TFF, and other methods known in the art, such as a falling film evaporator, may be used to concentrate the solution following removal of the dissolution solvent (e.g., LiBr) (with resulting desired concentration ranging from 0.1% to 30% silk). This can be used as an alternative to standard HFIP concentration methods known in the art to create a water-based solution. A larger pore membrane could also be utilized to filter out small silk protein fragments and to create a solution of higher molecular weight silk with and/or without tighter polydispersity values.

An assay for LiBr and Na₂CO₃ detection can be performed using an HPLC system equipped with evaporative light scattering detector (ELSD). The calculation was performed by linear regression of the resulting peak areas for the analyte plotted against concentration. More than one sample of a number of formulations of the present disclosure was used for sample preparation and analysis. Generally, four samples of different formulations were weighed directly in a 10 mL volumetric flask. The samples were suspended in 5 mL of 20 mM ammonium formate (pH 3.0) and kept at 2-8° C. for 2 hours with occasional shaking to extract analytes from the film. After 2 hours the solution was diluted with 20 mM ammonium formate (pH 3.0). The sample solution from the volumetric flask was transferred into HPLC vials and injected into the HPLC-ELSD system for the estimation of sodium carbonate and lithium bromide.

The analytical method developed for the quantitation of Na₂CO₃ and LiBr in silk protein formulations was found to be linear in the range 10-165 μg/mL, with RSD for injection precision as 2% and 1% for area and 0.38% and 0.19% for retention time for sodium carbonate and lithium bromide respectively. The analytical method can be applied for the quantitative determination of sodium carbonate and lithium bromide in silk protein formulations.

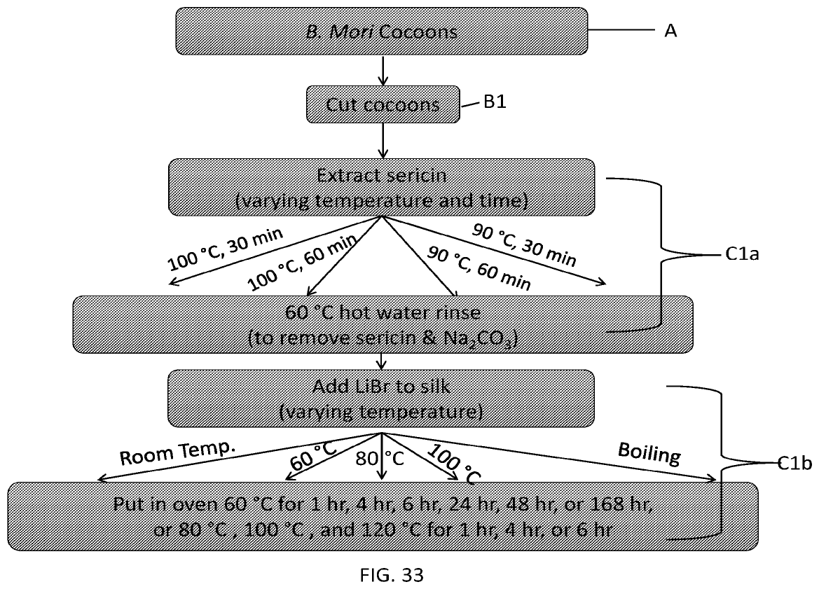

FIG. 33 is a flow chart showing various parameters that can be modified during the process of producing a silk protein fragment solution of the present disclosure during the extraction and the dissolution steps. Select method parameters may be altered to achieve distinct final solution characteristics depending upon the intended use, e.g., molecular weight and polydispersity. It should be understood that not all of the steps illustrated are necessarily required to fabricate all silk solutions of the present disclosure.

In an embodiment, silk protein fragment solutions useful for a wide variety of applications are prepared according to the following steps: forming pieces of silk cocoons from the Bombyx mori silkworm; extracting the pieces at about 100° C. in a Na₂CO₃ water solution for about 60 minutes, wherein a volume of the water equals about 0.4× raw silk weight and the amount of Na₂CO₃ is about 0.848× the weight of the pieces to form a silk fibroin extract; triple rinsing the silk fibroin extract at about 60° C. for about 20 minutes per rinse in a volume of rinse water, wherein the rinse water for each cycle equals about 0.2 L×the weight of the pieces; removing excess water from the silk fibroin extract; drying the silk fibroin extract; dissolving the dry silk fibroin extract in a LiBr solution, wherein the LiBr solution is first heated to about 100° C. to create a silk and LiBr solution and maintained; placing the silk and LiBr solution in a dry oven at about 100° C. for about 60 minutes to achieve complete dissolution and further fragmentation of the native silk protein structure into mixture with desired molecular weight and polydispersity; filtering the solution to remove any remaining debris from the silkworm; diluting the solution with water to result in a 1.0 wt. % silk solution; and removing solvent from the solution using Tangential Flow Filtration (TFF). In an embodiment, a 10 kDa membrane is utilized to purify the silk solution and create the final desired silk-to-water ratio. TFF can then be used to further concentrate the silk solution to a concentration of 2.0 wt. % silk in water.

Without wishing to be bound by any particular theory, varying extraction (i.e., time and temperature), LiBr (i.e., temperature of LiBr solution when added to silk fibroin extract or vice versa) and dissolution (i.e., time and temperature) parameters results in solvent and silk solutions with different viscosities, homogeneities, and colors. Also without wishing to be bound by any particular theory, increasing the temperature for extraction, lengthening the extraction time, using a higher temperature LiBr solution at emersion and over time when dissolving the silk and increasing the time at temperature (e.g., in an oven as shown here, or an alternative heat source) all resulted in less viscous and more homogeneous solvent and silk solutions.

The extraction step could be completed in a larger vessel, for example an industrial washing machine where temperatures at or in between 60° C. to 100° C. can be maintained. The rinsing step could also be completed in the industrial washing machine, eliminating the manual rinse cycles. Dissolution of the silk in LiBr solution could occur in a vessel other than a convection oven, for example a stirred tank reactor. Dialyzing the silk through a series of water changes is a manual and time intensive process, which could be accelerated by changing certain parameters, for example diluting the silk solution prior to dialysis. The dialysis process could be scaled for manufacturing by using semi-automated equipment, for example a tangential flow filtration system.

Varying extraction (i.e., time and temperature), LiBr (i.e., temperature of LiBr solution when added to silk fibroin extract or vice versa) and dissolution (i.e., time and temperature) parameters results in solvent and silk solutions with different viscosities, homogeneities, and colors. Increasing the temperature for extraction, lengthening the extraction time, using a higher temperature LiBr solution at emersion and over time when dissolving the silk and increasing the time at temperature (e.g., in an oven as shown here, or an alternative heat source) all resulted in less viscous and more homogeneous solvent and silk solutions. While almost all parameters resulted in a viable silk solution, methods that allow complete dissolution to be achieved in fewer than 4 to 6 hours are preferred for process scalability.

In an embodiment, solutions of silk fibroin protein fragments having a weight average selected from between about 6 kDa to about 17 kDa are prepared according to following steps: degumming a silk source by adding the silk source to a boiling (100° C.) aqueous solution of sodium carbonate for a treatment time of between about 30 minutes to about 60 minutes; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 60° C. to about 140° C.; maintaining the solution of silk fibroin-lithium bromide in an oven having a temperature of about 140° C. for a period of at most 1 hour; removing the lithium bromide from the silk fibroin extract; and producing an aqueous solution of silk protein fragments, the aqueous solution comprising: fragments having a weight average molecular weight selected from between about 6 kDa to about 17 kDa, and a polydispersity of between 1 and about 5, or between about 1.5 and about 3.0. The method may further comprise drying the silk fibroin extract prior to the dissolving step. The aqueous solution of silk fibroin protein fragments may comprise lithium bromide residuals of less than 300 ppm as measured using a high-performance liquid chromatography lithium bromide assay. The aqueous solution of silk fibroin protein fragments may comprise sodium carbonate residuals of less than 100 ppm as measured using a high-performance liquid chromatography sodium carbonate assay. The aqueous solution of silk fibroin protein fragments may be lyophilized. In some embodiments, the silk fibroin protein fragment solution may be further processed into various forms including gel, powder, and nanofiber.

In an embodiment, solutions of silk fibroin protein fragments having a weight average molecular weight selected from between about 17 kDa to about 39 kDa are prepared according to the following steps: adding a silk source to a boiling (100° C.) aqueous solution of sodium carbonate for a treatment time of between about 30 minutes to about 60 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at most 1 hour; removing the lithium bromide from the silk fibroin extract; and producing an aqueous solution of silk fibroin protein fragments, wherein the aqueous solution of silk fibroin protein fragments comprises lithium bromide residuals of between about 10 ppm and about 300 ppm, wherein the aqueous solution of silk protein fragments comprises sodium carbonate residuals of between about 10 ppm and about 100 ppm, wherein the aqueous solution of silk fibroin protein fragments comprises fragments having a weight average molecular weight selected from between about 17 kDa to about 39 kDa, and a polydispersity of between 1 and about 5, or between about 1.5 and about 3.0. The method may further comprise drying the silk fibroin extract prior to the dissolving step. The aqueous solution of silk fibroin protein fragments may comprise lithium bromide residuals of less than 300 ppm as measured using a high-performance liquid chromatography lithium bromide assay. The aqueous solution of silk fibroin protein fragments may comprise sodium carbonate residuals of less than 100 ppm as measured using a high-performance liquid chromatography sodium carbonate assay.

In some embodiments, a method for preparing an aqueous solution of silk fibroin protein fragments having an average weight average molecular weight selected from between about 6 kDa to about 17 kDa includes the steps of: degumming a silk source by adding the silk source to a boiling (100° C.) aqueous solution of sodium carbonate for a treatment time of between about 30 minutes to about 60 minutes; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 60° C. to about 140° C.; maintaining the solution of silk fibroin-lithium bromide in an oven having a temperature of about 140° C. for a period of at least 1 hour; removing the lithium bromide from the silk fibroin extract; and producing an aqueous solution of silk protein fragments, the aqueous solution comprising: fragments having an average weight average molecular weight selected from between about 6 kDa to about 17 kDa, and a polydispersity of between 1 and about 5, or between about 1.5 and about 3.0. The method may further comprise drying the silk fibroin extract prior to the dissolving step. The aqueous solution of pure silk fibroin protein fragments may comprise lithium bromide residuals of less than 300 ppm as measured using a high-performance liquid chromatography lithium bromide assay. The aqueous solution of pure silk fibroin protein fragments may comprise sodium carbonate residuals of less than 100 ppm as measured using a high-performance liquid chromatography sodium carbonate assay. The method may further comprise adding a therapeutic agent to the aqueous solution of pure silk fibroin protein fragments. The method may further comprise adding a molecule selected from one of an antioxidant or an enzyme to the aqueous solution of pure silk fibroin protein fragments. The method may further comprise adding a vitamin to the aqueous solution of pure silk fibroin protein fragments. The vitamin may be vitamin C or a derivative thereof. The aqueous solution of pure silk fibroin protein fragments may be lyophilized. The method may further comprise adding an alpha hydroxy acid to the aqueous solution of pure silk fibroin protein fragments. The alpha hydroxy acid may be selected from the group consisting of glycolic acid, lactic acid, tartaric acid and citric acid. The method may further comprise adding hyaluronic acid or its salt form at a concentration of about 0.5% to about 10.0% to the aqueous solution of pure silk fibroin protein fragments. The method may further comprise adding at least one of zinc oxide or titanium dioxide. A film may be fabricated from the aqueous solution of pure silk fibroin protein fragments produced by this method. The film may comprise from about 1.0 wt. % to about 50.0 wt. % of vitamin C or a derivative thereof. The film may have a water content ranging from about 2.0 wt. % to about 20.0 wt. %. The film may comprise from about 30.0 wt. % to about 99.5 wt. % of pure silk fibroin protein fragments. A gel may be fabricated from the aqueous solution of pure silk fibroin protein fragments produced by this method. The gel may comprise from about 0.5 wt. % to about 20.0 wt. % of vitamin C or a derivative thereof. The gel may have a silk content of at least 2% and a vitamin content of at least 20%.

In some embodiments, a method for preparing an aqueous solution of silk fibroin protein fragments having an average weight average molecular weight selected from between about 17 kDa to about 39 kDa includes the steps of: adding a silk source to a boiling (100° C.) aqueous solution of sodium carbonate for a treatment time of between about 30 minutes to about 60 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at least 1 hour; removing the lithium bromide from the silk fibroin extract; and producing an aqueous solution of pure silk fibroin protein fragments, wherein the aqueous solution of pure silk fibroin protein fragments comprises lithium bromide residuals of between about 10 ppm and about 300 ppm, wherein the aqueous solution of silk protein fragments comprises sodium carbonate residuals of between about 10 ppm and about 100 ppm, wherein the aqueous solution of pure silk fibroin protein fragments comprises fragments having an average weight average molecular weight selected from between about 17 kDa to about 39 kDa, and a polydispersity of between 1 and about 5, or between about 1.5 and about 3.0. The method may further comprise drying the silk fibroin extract prior to the dissolving step. The aqueous solution of pure silk fibroin protein fragments may comprise lithium bromide residuals of less than 300 ppm as measured using a high-performance liquid chromatography lithium bromide assay. The aqueous solution of pure silk fibroin protein fragments may comprise sodium carbonate residuals of less than 100 ppm as measured using a high-performance liquid chromatography sodium carbonate assay. The method may further comprise adding a therapeutic agent to the aqueous solution of pure silk fibroin protein fragments. The method may further comprise adding a molecule selected from one of an antioxidant or an enzyme to the aqueous solution of pure silk fibroin protein fragments. The method may further comprise adding a vitamin to the aqueous solution of pure silk fibroin protein fragments. The vitamin may be vitamin C or a derivative thereof. The aqueous solution of pure silk fibroin protein fragments may be lyophilized. The method may further comprise adding an alpha hydroxy acid to the aqueous solution of pure silk fibroin protein fragments. The alpha hydroxy acid may be selected from the group consisting of glycolic acid, lactic acid, tartaric acid and citric acid. The method may further comprise adding hyaluronic acid or its salt form at a concentration of about 0.5% to about 10.0% to the aqueous solution of pure silk fibroin protein fragments. The method may further comprise adding at least one of zinc oxide or titanium dioxide. A film may be fabricated from the aqueous solution of pure silk fibroin protein fragments produced by this method. The film may comprise from about 1.0 wt. % to about 50.0 wt. % of vitamin C or a derivative thereof. The film may have a water content ranging from about 2.0 wt. % to about 20.0 wt. %. The film may comprise from about 30.0 wt. % to about 99.5 wt. % of pure silk fibroin protein fragments. A gel may be fabricated from the aqueous solution of pure silk fibroin protein fragments produced by this method. The gel may comprise from about 0.5 wt. % to about 20.0 wt. % of vitamin C or a derivative thereof. The gel may have a silk content of at least 2% and a vitamin content of at least 20%.

In an embodiment, solutions of silk fibroin protein fragments having a weight average molecular weight selected from between about 39 kDa to about 80 kDa are prepared according to the following steps: adding a silk source to a boiling (100° C.) aqueous solution of sodium carbonate for a treatment time of about 30 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at most 1 hour; removing the lithium bromide from the silk fibroin extract; and producing an aqueous solution of silk fibroin protein fragments, wherein the aqueous solution of silk fibroin protein fragments comprises lithium bromide residuals of between about 10 ppm and about 300 ppm, sodium carbonate residuals of between about 10 ppm and about 100 ppm, fragments having a weight average molecular weight selected from between about 39 kDa to about 80 kDa, and a polydispersity of between 1 and about 5, or between about 1.5 and about 3.0. The method may further comprise drying the silk fibroin extract prior to the dissolving step. The aqueous solution of silk fibroin protein fragments may comprise lithium bromide residuals of less than 300 ppm as measured using a high-performance liquid chromatography lithium bromide assay. The aqueous solution of silk fibroin protein fragments may comprise sodium carbonate residuals of less than 100 ppm as measured using a high-performance liquid chromatography sodium carbonate assay. In some embodiments, the method may further comprise adding an active agent (e.g., therapeutic agent) to the aqueous solution of pure silk fibroin protein fragments. The method may further comprise adding an active agent selected from one of an antioxidant or an enzyme to the aqueous solution of pure silk fibroin protein fragments. The method may further comprise adding a vitamin to the aqueous solution of pure silk fibroin protein fragments. The vitamin may be vitamin C or a derivative thereof. The aqueous solution of pure silk fibroin protein fragments may be lyophilized. The method may further comprise adding an alpha-hydroxy acid to the aqueous solution of pure silk fibroin protein fragments. The alpha hydroxy acid may be selected from the group consisting of glycolic acid, lactic acid, tartaric acid and citric acid. The method may further comprise adding hyaluronic acid or its salt form at a concentration of about 0.5% to about 10.0% to the aqueous solution of pure silk fibroin protein fragments. A film may be fabricated from the aqueous solution of pure silk fibroin protein fragments produced by this method. The film may comprise from about 1.0 wt. % to about 50.0 wt. % of vitamin C or a derivative thereof. The film may have a water content ranging from about 2.0 wt. % to about 20.0 wt. %. The film may comprise from about 30.0 wt. % to about 99.5 wt. % of pure silk fibroin protein fragments. A gel may be fabricated from the aqueous solution of pure silk fibroin protein fragments produced by this method. The gel may comprise from about 0.5 wt. % to about 20.0 wt. % of vitamin C or a derivative thereof. The gel may have a silk content of at least 2 wt. % and a vitamin content of at least 20 wt. %.

Molecular weight of the silk protein fragments may be controlled based upon the specific parameters utilized during the extraction step, including extraction time and temperature; specific parameters utilized during the dissolution step, including the LiBr temperature at the time of submersion of the silk in to the lithium bromide and time that the solution is maintained at specific temperatures; and specific parameters utilized during the filtration step. By controlling process parameters using the disclosed methods, it is possible to create silk fibroin protein fragment solutions with polydispersity equal to or lower than 2.5 at a variety of different molecular weight selected from between 5 kDa to 200 kDa, or between 10 kDa and 80 kDa. By altering process parameters to achieve silk solutions with different molecular weights, a range of fragment mixture end products, with desired polydispersity of equal to or less than 2.5 may be targeted based upon the desired performance requirements. For example, a higher molecular weight silk film containing an ophthalmic drug may have a controlled slow release rate compared to a lower molecular weight film making it ideal for a delivery vehicle in eye care products. Additionally, the silk fibroin protein fragment solutions with a polydispersity of greater than 2.5 can be achieved. Further, two solutions with different average molecular weights and polydispersity can be mixed to create combination solutions. Alternatively, a liquid silk gland (100% sericin free silk protein) that has been removed directly from a worm could be used in combination with any of the silk fibroin protein fragment solutions of the present disclosure. Molecular weight of the pure silk fibroin protein fragment composition was determined using High Pressure Liquid Chromatography (HPLC) with a Refractive Index Detector (RID). Polydispersity was calculated using Cirrus GPC Online GPC/SEC Software Version 3.3 (Agilent).

Differences in the processing parameters can result in regenerated silk fibroins that vary in molecular weight, and peptide chain size distribution (polydispersity, PD). This, in turn, influences the regenerated silk fibroin performance, including mechanical strength, water solubility etc.

Parameters were varied during the processing of raw silk cocoons into the silk solution. Varying these parameters affected the MW of the resulting silk solution. Parameters manipulated included (i) time and temperature of extraction, (ii) temperature of LiBr, (iii) temperature of dissolution oven, and (iv) dissolution time. Experiments were carried out to determine the effect of varying the extraction time. Tables A-G summarize the results. Below is a summary:
A sericin extraction time of 30 minutes resulted in larger molecular weight than a sericin extraction time of 60 minutes
Molecular weight decreases with time in the oven
140° C. LiBr and oven resulted in the low end of the confidence interval to be below a molecular weight of 9500 Da
30 min extraction at the 1 hour and 4 hour time points have undigested silk
30 min extraction at the 1 hour time point resulted in a significantly high molecular weight with the low end of the confidence interval being 35,000 Da
The range of molecular weight reached for the high end of the confidence interval was 18000 to 216000 Da (important for offering solutions with specified upper limit).

TABLE A
The effect of extraction time (30 min vs 60 min)
on molecular weight of silk processed under the
conditions of 100° C. Extraction Temperature,
100° C. Lithium Bromide (LiBr) and 100° C. Oven
Dissolution (Oven/Dissolution Time was varied).

| Boil | Oven | Average | | | | |
|---|---|---|---|---|---|---|
| Time | Time | Mw | Std dev | Confidence Interval | | PD |
| 30 | 1 | 57247 | 12780 | 35093 | 93387 | 1.63 |
| 60 | 1 | 31520 | 1387 | 11633 | 85407 | 2.71 |
| 30 | 4 | 40973 | 2632 | 14268 | 117658 | 2.87 |
| 60 | 4 | 25082 | 1248 | 10520 | 59803 | 2.38 |
| 30 | 6 | 25604 | 1405 | 10252 | 63943 | 2.50 |
| 60 | 6 | 20980 | 1262 | 10073 | 43695 | 2.08 |

TABLE B
The effect of extraction time (30 min vs 60 min) on
molecular weight of silk processed under the conditions
of 100° C. Extraction Temperature, boiling Lithium
Bromide (LiBr) and 60° C. Oven Dissolution for 4 hr.

| | Boil | Average | Std | | | |
|---|---|---|---|---|---|---|
| Sample | Time | Mw | dev | Confidence Interval | | PD |
| 30 min, 4 hr | 30 | 49656 | 4580 | 17306 | 142478 | 2.87 |
| 60 min, 4 hr | 60 | 30042 | 1536 | 11183 | 80705 | 2.69 |

TABLE C
The effect of extraction time (30 min vs 60 min) on molecular
weight of silk processed under the conditions of 100° C. Extraction
Temperature, 60° C. Lithium Bromide (LiBr) and 60° C. Oven
Dissolution (Oven/Dissolution Time was varied).

| | Boil | Oven | Average | Std | | | |
|---|---|---|---|---|---|---|---|
| Sample | Time | Time | Mw | dev | Confidence Interval | | PD |
| 30 min, 1 hr | 30 | 1 | 58436 | | 22201 | 153809 | 2.63 |
| 60 min, 1 hr | 60 | 1 | 31700 | | 11931 | 84224 | 2.66 |
| 30 min, 4 hr | 30 | 4 | 61956.5 | 13337 | 21463 | 178847 | 2.89 |
| 60 min, 4 hr | 60 | 4 | 25578.5 | 2446 | 9979 | 65564 | 2.56 |

TABLE D
The effect of extraction time (30 min vs 60 min) on molecular weight of silk processed
under the conditions of 100° C. Extraction Temperature, 80° C. Lithium Bromide (LiBr)
and 80° C. Oven Dissolution for 6 hr.

| | Boil | Average | Std | Confidence | | |
|---|---|---|---|---|---|---|
| Sample | Time | Mw | dev | Interval | | PD |
| 30 min, 6 hr | 30 | 63510 | | 18693 | 215775 | 3.40 |
| 60 min, 6 hr | 60 | 25164 | 238 | 9637 | 65706 | 2.61 |

TABLE E
The effect of extraction time (30 min vs 60 min) on molecular weight of silk processed
under the conditions of 100° C. Extraction Temperature, 80° C. Lithium Bromide (LiBr)
and 60° C. Oven Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 30 min, 4 hr | 30 | 4 | 59202 | 14028 | 19073 | 183760 | 3.10 |
| 60 min, 4 hr | 60 | 4 | 26312.5 | 637 | 10266 | 67442 | 2.56 |
| 30 min, 6 hr | 30 | 6 | 46824 | | 18076 | 121293 | 2.59 |
| 60 min, 6 hr | 60 | 6 | 26353 | | 10168 | 68302 | 2.59 |

TABLE F
The effect of extraction time (30 min vs 60 min) on molecular weight of silk processed
under the conditions of 100° C. Extraction Temperature, 140° C. Lithium Bromide (LiBr)
and 140° C. Oven Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 30 min, 4 hr | 30 | 4 | 9024.5 | 1102 | 4493 | 18127 | 2.00865 |
| 60 min, 4 hr | 60 | 4 | 15548 | | 6954 | 34762 | 2.2358 |
| 30 min, 6 hr | 30 | 6 | 13021 | | 5987 | 28319 | 2.1749 |
| 60 min, 6 hr | 60 | 6 | 10888 | | 5364 | 22100 | 2.0298 |

Experiments were carried out to determine the effect of varying the extraction temperature. Table G summarizes the results. Below is a summary:
Sericin extraction at 90° C. resulted in higher MW than sericin extraction at 100° C. extraction
Both 90° C. and 100° C. show decreasing MW over time in the oven.

TABLE G
The effect of extraction temperature (90° C. vs. 100° C.) on molecular weight of silk
processed under the conditions of 60 min. Extraction Temperature, 100° C. Lithium
Bromide (LiBr) and 100° C. Oven Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 90° C., 4 hr | 60 | 4 | 37308 | 4204 | 13368 | 104119 | 2.79 |
| 100° C., 4 hr | 60 | 4 | 25082 | 1248 | 10520 | 59804 | 2.38 |
| 90° C., 6 hr | 60 | 6 | 34224 | 1135 | 12717 | 92100 | 2.69 |
| 100° C., 6 hr | 60 | 6 | 20980 | 1262 | 10073 | 43694 | 2.08 |

Experiments were carried out to determine the effect of varying the Lithium Bromide (LiBr) temperature when added to silk. Tables H-I summarize the results. Below is a summary:
No impact on molecular weight or confidence interval (all CI˜10500-6500 Da)
Studies illustrated that the temperature of LiBr-silk dissolution, as LiBr is added and begins dissolving, rapidly drops below the original LiBr temperature due to the majority of the mass being silk at room temperature

TABLE H
The effect of Lithium Bromide (LiBr) temperature on molecular weight of silk
processed under the conditions of 60 min. Extraction Time., 100° C. Extraction
Temperature and 60° C. Oven Dissolution (Oven/Dissolution Time was varied).

| | LiBr | | | | | | |
|---|---|---|---|---|---|---|---|
| | Temp | Oven | Average | Std | Confidence | | |
| Sample | (° C.) | Time | Mw | dev | Interval | | PD |
| 60° C. LiBr, | 60 | 1 | 31700 | | 11931 | 84223 | 2.66 |
| 1 hr | | | | | | | |
| 100° C. LiBr, | 100 | 1 | 27907 | 200 | 10735 | 72552 | 2.60 |
| 1 hr | | | | | | | |
| RT LiBr, | RT | 4 | 29217 | 1082 | 10789 | 79119 | 2.71 |
| 4 hr | | | | | | | |
| 60° C. LiBr, | 60 | 4 | 25578 | 2445 | 9978 | 65564 | 2.56 |
| 4 hr | | | | | | | |
| 80° C. LiBr, | 80 | 4 | 26312 | 637 | 10265 | 67441 | 2.56 |
| 4 hr | | | | | | | |
| 100° C. LiBr, | 100 | 4 | 27681 | 1729 | 11279 | 67931 | 2.45 |
| 4 hr | | | | | | | |
| Boil LiBr, | Boil | 4 | 30042 | 1535 | 11183 | 80704 | 2.69 |
| 4 hr | | | | | | | |
| RT LiBr, | RT | 6 | 26543 | 1893 | 10783 | 65332 | 2.46 |
| 6 hr | | | | | | | |
| 80° C. LiBr, | 80 | 6 | 26353 | | 10167 | 68301 | 2.59 |
| 6 hr | | | | | | | |
| 100° C. LiBr, | 100 | 6 | 27150 | 916 | 11020 | 66889 | 2.46 |
| 6 hr | | | | | | | |

TABLE I
The effect of Lithium Bromide (LiBr) temperature on molecular weight of silk
processed under the conditions of 30 min. Extraction Time, 100° C. Extraction
Temperature and 60° C. Oven Dissolution (Oven/Dissolution Time was varied).

| | LiBr | | | | | | |
|---|---|---|---|---|---|---|---|
| | Temp | Oven | Average | Std | Confidence | | |
| Sample | (° C.) | Time | Mw | dev | Interval | | PD |
| 60° C. LiBr, | 60 | 4 | 61956 | 13336 | 21463 | 178847 | 2.89 |
| 4 hr | | | | | | | |
| 80° C. LiBr, | 80 | 4 | 59202 | 14027 | 19073 | 183760 | 3.10 |
| 4 hr | | | | | | | |
| 100° C. LiBr, | 100 | 4 | 47853 | | 19757 | 115899 | 2.42 |
| 4 hr | | | | | | | |
| 80° C. LiBr, | 80 | 6 | 46824 | | 18075 | 121292 | 2.59 |
| 6 hr | | | | | | | |
| 100° C. LiBr, | 100 | 6 | 55421 | 8991 | 19152 | 160366 | 2.89 |
| 6 hr | | | | | | | |

Experiments were carried out to determine the effect of v oven/dissolution temperature. Tables J-N summarize the results. Below is a summary:
Oven temperature has less of an effect on 60 min extracted silk than 30 min extracted silk. Without wishing to be bound by theory, it is believed that the 30 min silk is less degraded during extraction and therefore the oven temperature has more of an effect on the larger MW, less degraded portion of the silk.
For 60° C. vs. 140° C. oven the 30 min extracted silk showed a very significant effect of lower MW at higher oven temp, while 60 min extracted silk had an effect but much less
The 140° C. oven resulted in a low end in the confidence interval at ˜6000 Da.

TABLE J
The effect of oven/dissolution temperature on molecular weight of silk
processed under the conditions of 100° C. Extraction Temperature, 30 min. Extraction
Time, and 100° C. Lithium Bromide (LiBr) (Oven/Dissolution Time was varied)

| Boil | Oven Temp | Oven | Average | Std | Confidence | | |
|---|---|---|---|---|---|---|---|
| Time | (° C.) | Time | Mw | dev | Interval | | PD |
| 30 | 60 | 4 | 47853 | | 19758 | 115900 | 2.42 |
| 30 | 100 | 4 | 40973 | 2632 | 14268 | 117658 | 2.87 |
| 30 | 60 | 6 | 55421 | 8992 | 19153 | 160366 | 2.89 |
| 30 | 100 | 6 | 25604 | 1405 | 10252 | 63943 | 2.50 |

TABLE K
The effect of oven/dissolution temperature on molecular weight of silk
processed under the conditions of 100° C. Extraction Temperature, 60 min. Extraction
Time, and 100° C. Lithium Bromide (LiBr) (Oven/Dissolution Time was varied).

| Boil Time | Oven | Oven | Average | Std | Confidence | | |
|---|---|---|---|---|---|---|---|
| (minutes) | Temp | Time | Mw | dev | Interval | | PD |
| 60 | 60 | 1 | 27908 | 200 | 10735 | 72552 | 2.60 |
| 60 | 100 | 1 | 31520 | 1387 | 11633 | 85407 | 2.71 |
| 60 | 60 | 4 | 27681 | 1730 | 11279 | 72552 | 2.62 |
| 60 | 100 | 4 | 25082 | 1248 | 10520 | 59803 | 2.38 |
| 60 | 60 | 6 | 27150 | 916 | 11020 | 66889 | 2.46 |
| 60 | 100 | 6 | 20980 | 1262 | 10073 | 43695 | 2.08 |

TABLE L
The effect of oven/dissolution temperature on molecular weight of silk
processed under the conditions of 100° C. Extraction Temperature, 60 min. Extraction
Time, and 140° C. Lithium Bromide (LiBr) (Oven/Dissolution Time was varied).

| Boil Time | Oven | Oven | | Std | Confidence | | |
|---|---|---|---|---|---|---|---|
| (minutes) | Temp (° C.) | Time | Average | dev | Interval | | PD |
| 60 | 60 | 4 | 30042 | 1536 | 11183 | 80705 | 2.69 |
| 60 | | 4 | 15548 | | 7255 | 33322 | 2.14 |

TABLE M
The effect of oven/dissolution temperature on molecular weight of silk
processed under the conditions of 100° C. Extraction Temperature, 30 min. Extraction
Time, and 140° C. Lithium Bromide (LiBr) (Oven/Dissolution Time was varied).

| Boil Time | Oven | Oven | Average | Std | Confidence | | |
|---|---|---|---|---|---|---|---|
| (minutes) | Temp (° C.) | Time | Mw | dev | Interval | | PD |
| 30 | 60 | 4 | 49656 | 4580 | 17306 | 142478 | 2.87 |
| 30 | 140 | 4 | 9025 | 1102 | 4493 | 18127 | 2.01 |
| 30 | 60 | 6 | 59383 | 11640 | 17641 | 199889 | 3.37 |
| 30 | 140 | 6 | 13021 | | 5987 | 28319 | 2.17 |

TABLE N
The effect of oven/dissolution temperature on molecular weight of silk
processed under the conditions of 100° C. Extraction Temperature, 60 min. Extraction
Time, and 80° C. Lithium Bromide (LiBr) (Oven/Dissolution Time was varied).

| Boil Time | Oven | Oven | Average | Std | Confidence | | |
|---|---|---|---|---|---|---|---|
| (minutes) | Temp (° C.) | Time | Mw | dev | Interval | | PD |
| 60 | 60 | 4 | 26313 | 637 | 10266 | 67442 | 2.56 |
| 60 | 80 | 4 | 30308 | 4293 | 12279 | 74806 | 2.47 |
| 60 | 60 | 6 | 26353 | | 10168 | 68302 | 2.59 |
| 60 | 80 | 6 | 25164 | 238 | 9637 | 65706 | 2.61 |

The raw silk cocoons from the silkworm Bombyx mori was cut into pieces. The pieces of raw silk cocoons were boiled in an aqueous solution of Na₂CO₃ (about 100° C.) for a period of time between about 30 minutes to about 60 minutes to remove sericin (degumming). The volume of the water used equals about 0.4× raw silk weight and the amount of Na₂CO₃ is about 0.848× the weight of the raw silk cocoon pieces. The resulting degummed silk cocoon pieces were rinsed with deionized water three times at about 60° C. (20 minutes per rinse). The volume of rinse water for each cycle was 0.2 L×the weight of the raw silk cocoon pieces. The excess water from the degummed silk cocoon pieces was removed. After the DI water washing step, the wet degummed silk cocoon pieces were dried at room temperature. The degummed silk cocoon pieces were mixed with a LiBr solution, and the mixture was heated to about 100° C. The warmed mixture was placed in a dry oven and was heated at a temperature ranging from about 60° C. to about 140° C. for about 60 minutes to achieve complete dissolution of the native silk protein. The resulting solution was allowed to cool to room temperature and then was dialyzed to remove LiBr salts using a 3,500 Da MWCO membrane. Multiple exchanges were performed in Di water until Br⁻ ions were less than 1 ppm as determined in the hydrolyzed fibroin solution read on an Oakton Bromide (Br⁻) double junction ion-selective electrode.

The resulting silk fibroin aqueous solution has a concentration of about 8.0% w/v containing pure silk fibroin protein fragments having an average weight average molecular weight selected from between about 6 kDa to about 16 kDa, about 17 kDa to about 39 kDa, and about 39 kDa to about 80 kDa and a polydispersity of between about 1.5 and about 3.0. The 8.0% w/v was diluted with DI water to provide a 1.0% w/v, 2.0% w/v, 3.0% w/v, 4.0% w/v, 5.0% w/v by the coating solution.

A variety of % silk concentrations have been produced through the use of Tangential Flow Filtration (TFF). In all cases a 1% silk solution was used as the input feed. A range of 750-18,000 mL of 1% silk solution was used as the starting volume. Solution is diafiltered in the TFF to remove lithium bromide. Once below a specified level of residual LiBr, solution undergoes ultrafiltration to increase the concentration through removal of water. See examples below.

Six (6) silk solutions were utilized in standard silk structures with the following results:

Solution #1 is a silk concentration of 5.9 wt. %, average MW of 19.8 kDa and 2.2 PDI (made with a 60 min boil extraction, 100° C. LiBr dissolution for 1 hour).

Solution #2 is a silk concentration of 6.4 wt. % (made with a 30 min boil extraction, 60° C. LiBr dissolution for 4 hrs).

Solution #3 is a silk concentration of 6.17 wt. % (made with a 30 min boil extraction 100° C. LiBr dissolution for 1 hour).

Solution #4 is a silk concentration of 7.30 wt. %: A 7.30% silk solution was produced beginning with 30 minute extraction batches of 100 g silk cocoons per batch. Extracted silk fibers were then dissolved using 100° C. 9.3 M LiBr in a 100° C. oven for 1 hour. 100 g of silk fibers were dissolved per batch to create 20% silk in LiBr. Dissolved silk in LiBr was then diluted to 1% silk and filtered through a 5 μm filter to remove large debris. 15,500 mL of 1%, filtered silk solution was used as the starting volume/diafiltration volume for TFF. Once LiBr was removed, the solution was ultrafiltered to a volume around 1300 mL. 1262 mL of 7.30% silk was then collected. Water was added to the feed to help remove the remaining solution and 547 mL of 3.91% silk was then collected.

Solution #5 is a silk concentration of 6.44 wt. %: A 6.44 wt. % silk solution was produced beginning with 60 minute extraction batches of a mix of 25, 33, 50, 75 and 100 g silk cocoons per batch. Extracted silk fibers were then dissolved using 100° C. 9.3 M LiBr in a 100° C. oven for 1 hour. 35, 42, 50 and 71 g per batch of silk fibers were dissolved to create 20% silk in LiBr and combined. Dissolved silk in LiBr was then diluted to 1% silk and filtered through a 5 μm filter to remove large debris. 17,000 mL of 1%, filtered silk solution was used as the starting volume/diafiltration volume for TFF. Once LiBr was removed, the solution was ultrafiltered to a volume around 3000 mL. 1490 mL of 6.44% silk was then collected. Water was added to the feed to help remove the remaining solution and 1454 mL of 4.88% silk was then collected.

Solution #6 is a silk concentration of 2.70 wt. %: A 2.70% silk solution was produced beginning with 60-minute extraction batches of 25 g silk cocoons per batch. Extracted silk fibers were then dissolved using 100° C. 9.3 M LiBr in a 100° C. oven for 1 hour. 35.48 g of silk fibers were dissolved per batch to create 20% silk in LiBr. Dissolved silk in LiBr was then diluted to 1% silk and filtered through a 5 μm filter to remove large debris. 1000 mL of 1%, filtered silk solution was used as the starting volume/diafiltration volume for TFF. Once LiBr was removed, the solution was ultrafiltered to a volume around 300 mL. 312 mL of 2.7% silk was then collected.

The preparation of silk fibroin solutions with higher molecular weights is given in Table O.

TABLE O
Preparation and properties of silk fibroin solutions

| | Extraction | Extraction | LiBr | | | Average weight | |
|---|---|---|---|---|---|---|---|
| Sample | Time | Temp | Temp | Oven/Sol'n | | average molecular | Average |
| Name | (mins) | (° C.) | (° C.) | Temp | | weight (kDa) | polydispersity |
| Group A | 60 | 100 | 100 | 100° C. | oven | 34.7 | 2.94 |
| TFF | | | | | | | |
| Group A | 60 | 100 | 100 | 100° C. | oven | 44.7 | 3.17 |
| DIS | | | | | | | |
| Group B | 60 | 100 | 100 | 100° C. | sol'n | 41.6 | 3.07 |
| TFF | | | | | | | |
| Group B | 60 | 100 | 100 | 100° C. | sol'n | 44.0 | 3.12 |
| DIS | | | | | | | |
| Group D | 30 | 90 | 60 | 60° C. | sol'n | 129.7 | 2.56 |
| DIS | | | | | | | |
| Group D | 30 | 90 | 60 | 60° C. | sol'n | 144.2 | 2.73 |
| FIL | | | | | | | |
| Group E | 15 | 100 | RT | 60° C. | sol'n | 108.8 | 2.78 |
| DIS | | | | | | | |
| Group E | 15 | 100 | RT | 60° C. | sol'n | 94.8 | 2.62 |

FIL

Silk aqueous coating composition for application to fabrics are given in Tables P and Q below.

TABLE P
Silk Solution Characteristics

| Molecular Weight: | 57 kDa | | | |
|---|---|---|---|---|
| Polydispersity: | 1.6 | | | |
| % Silk | 5.0% | 3.0% | 1.0% | 0.5% |

Process Parameters

| Extraction | | |
|---|---|---|
| Boil Time: | 30 | minutes |
| Boil Temperature: | 100° | C. |
| Rinse Temperature: | 60° | C. |
| Dissolution | | |
| LiBr Temperature: | 100 | |
| Oven Temperature: | 100° | C. |
| Oven Time: | 60 | minutes |

TABLE Q
Silk Solution Characteristics

| Molecular Weight: | 25 kDa | | | |
|---|---|---|---|---|
| Polydispersity: | 2.4 | | | |
| % Silk | 5.0% | 3.0% | 1.0% | 0.5% |

Process Parameters

| | | |
|---|---|---|
| Extraction | | |
| Boil Time: | 60 | minutes |
| Boil Temperature: | 100° | C. |
| Rinse Temperature: | 60° | C. |
| Dissolution | | |
| LiBr Temperature: | 100° | C. |
| Oven Temperature: | 100° | C. |
| Oven Time: | 60 | minutes |

Three (3) silk solutions were utilized in film making with the following results:

Solution #1 is a silk concentration of 5.9%, average MW of 19.8 kDa and 2.2 PD (made with a 60 min boil extraction, 100° C. LiBr dissolution for 1 hr).

Solution #2 is a silk concentration of 6.4% (made with a 30 min boil extraction, 60° C. LiBr dissolution for 4 hrs).

Solution #3 is a silk concentration of 6.17% (made with a 30 min boil extraction, 100° C. LiBr dissolution for 1 hour).

Films were made in accordance with Rockwood et al. (Nature Protocols; Vol. 6; No. 10; published on-line Sep. 22, 2011; doi:10.1038/nprot.2011.379). 4 mL of 1% or 2% (wt/vol) aqueous silk solution was added into 100 mm Petri dish (Volume of silk can be varied for thicker or thinner films and is not critical) and allowed to dry overnight uncovered. The bottom of a vacuum desiccator was filled with water. Dry films were placed in the desiccator and vacuum applied, allowing the films to water anneal for 4 hours prior to removal from the dish. Films cast from solution #1 did not result in a structurally continuous film; the film was cracked in several pieces. These pieces of film dissolved in water in spite of the water annealing treatment.

Silk solutions of various molecular weights and/or combinations of molecular weights can be optimized for gel applications. The following provides an example of this process but it not intended to be limiting in application or formulation. Three (3) silk solutions were utilized in gel making with the following results:

Solution #1 is a silk concentration of 5.9%, average MW of 19.8 kDa and 2.2 PD (made with a 60 min boil extraction, 100° C. LiBr dissolution for 1 hr).

Solution #2 is a silk concentration of 6.4% (made with a 30 min boil extraction, 60° C. LiBr dissolution for 4 hrs).

Solution #3 is a silk concentration of 6.17% (made with a 30 min boil extraction, 100° C. LiBr dissolution for 1 hour).

“Egel” is an electrogelation process as described in Rockwood of al. Briefly, 10 ml of aqueous silk solution is added to a 50 ml conical tube and a pair of platinum wire electrodes immersed into the silk solution. A 20 volt potential was applied to the platinum electrodes for 5 minutes, the power supply turned off and the gel collected. Solution #1 did not form an EGEL over the 5 minutes of applied electric current.

Solutions #2 and #3 were gelled in accordance with the published horseradish peroxidase (HRP) protocol. Behavior seemed typical of published solutions.

Materials and Methods: the following equipment and material are used in determination of Silk Molecular weight: Agilent 1100 with chemstation software ver. 10.01; Refractive Index Detector (RID); analytical balance; volumetric flasks (1000 mL, 10 mL and 5 mL); HPLC grade water; ACS grade sodium chloride; ACS grade sodium phosphate dibasic heptahydrate; phosphoric acid; dextran MW Standards-Nominal Molecular Weights of 5 kDa, 11.6 kDa, 23.8 kDa, 48.6 kDa, and 148 kDa; 50 mL PET or polypropylene disposable centrifuge tubes; graduated pipettes; amber glass HPLC vials with Teflon caps; Phenomenex PolySep GFC P-4000 column (size: 7.8 mm×300 mm).

### Procedural Steps:

### A) Preparation of 1 L Mobile Phase (0.1 M Sodium Chloride Solution in 0.0125 M Sodium Phosphate Buffer)

Take a 250 mL clean and dry beaker, place it on the balance and tare the weight. Add about 3.3509 g of sodium phosphate dibasic heptahydrate to the beaker. Note down the exact weight of sodium phosphate dibasic weighed. Dissolve the weighed sodium phosphate by adding 100 mL of HPLC water into the beaker. Take care not to spill any of the content of the beaker. Transfer the solution carefully into a clean and dry 1000 mL volumetric flask. Rinse the beaker and transfer the rinse into the volumetric flask. Repeat the rinse 4-5 times. In a separate clean and dry 250 mL beaker weigh exactly about 5.8440 g of sodium chloride. Dissolve the weighed sodium chloride in 50 mL of water and transfer the solution to the sodium phosphate solution in the volumetric flask. Rinse the beaker and transfer the rinse into the volumetric flask. Adjust the pH of the solution to 7.0±0.2 with phosphoric acid. Make up the volume in volumetric flask with HPLC water to 1000 mL and shake it vigorously to homogeneously mix the solution. Filter the solution through 0.45 μm polyamide membrane filter. Transfer the solution to a clean and dry solvent bottle and label the bottle. The volume of the solution can be varied to the requirement by correspondingly varying the amount of sodium phosphate dibasic heptahydrate and sodium chloride.

### B) Preparation of Dextran Molecular Weight Standard Solutions

At least five different molecular weight standards are used for each batch of samples that are run so that the expected value of the sample to be tested is bracketed by the value of the standard used. Label six 20 mL scintillation glass vials respective to the molecular weight standards. Weigh accurately about 5 mg of each of dextran molecular weight standards and record the weights. Dissolve the dextran molecular weight standards in 5 mL of mobile phase to make a 1 mg/mL standard solution.

### C) Preparation of Sample Solutions

When preparing sample solutions, if there are limitations on how much sample is available, the preparations may be scaled as long as the ratios are maintained. Depending on sample type and silk protein content in sample weigh enough sample in a 50 mL disposable centrifuge tube on an analytical balance to make a 1 mg/mL sample solution for analysis. Dissolve the sample in equivalent volume of mobile phase make a 1 mg/mL solution. Tightly cap the tubes and mix the samples (in solution). Leave the sample solution for 30 minutes at room temperature. Gently mix the sample solution again for 1 minute and centrifuge at 4000 RPM for 10 minutes.

### D) HPLC Analysis of the Samples

Transfer 1.0 mL of all the standards and sample solutions into individual HPLC vials. Inject the molecular weight standards (one injection each) and each sample in duplicate. Analyze all the standards and sample solutions using the following HPLC conditions:

| | |
|---|---|
| Column | PolySep GFC P-4000 (7.8 × 300 mm) |
| Column Temperature | 25° C. |
| Detector | Refractive Index Detector (Temperature @ |
| | 35° C.) |
| Injection Volume | 25.0 μL |
| Mobile Phase | 0.1M Sodium Chloride solution in 0.0125M |
| | sodium phosphate buffer |
| Flow Rate | 1.0 mL/min |
| Run Time | 20.0 min |

### E) Data Analysis and Calculations—Calculation of Average Molecular Weight Using Cirrus Software

Upload the chromatography data files of the standards and the analytical samples into Cirrus SEC data collection and molecular weight analysis software. Calculate the weight average molecular weight (M_{w}), number average molecular weight (Mₙ), peak average molecular weight (Mₚ), and polydispersity for each injection of the sample.

### Spider Silk Fragments

Spider silks are natural polymers that consist of three domains: a repetitive middle core domain that dominates the protein chain, and non-repetitive N-terminal and C-terminal domains. The large core domain is organized in a block copolymer-like arrangement, in which two basic sequences, crystalline [poly(A) or poly(GA)] and less crystalline (GGX or GPGXX (SEQ ID NO: 6)) polypeptides alternate. Dragline silk is the protein complex composed of major ampullate dragline silk protein 1 (MaSp1) and major ampullate dragline silk protein 2 (MaSp2). Both silks are approximately 3500 amino acid long. MaSp1 can be found in the fibre core and the periphery, whereas MaSp2 forms clusters in certain core areas. The large central domains of MaSp1 and MaSp2 are organized in block copolymer-like arrangements, in which two basic sequences, crystalline [poly(A) or poly(GA)] and less crystalline (GGX or GPGXX (SEQ ID NO: 6)) polypeptides alternate in core domain. Specific secondary structures have been assigned to poly(A)/(GA), GGX and GPGXX (SEQ ID NO: 6) motifs including β-sheet, α-helix and β-spiral respectively. The primary sequence, composition and secondary structural elements of the repetitive core domain are responsible for mechanical properties of spider silks; whereas, non-repetitive N- and C-terminal domains are essential for the storage of liquid silk dope in a lumen and fibre formation in a spinning duct.

The main difference between MaSp1 and MaSp2 is the presence of proline (P) residues accounting for 15% of the total amino acid content in MaSp2, whereas MaSp1 is proline-free. By calculating the number of proline residues in N. clavipes dragline silk, it is possible to estimate the presence of the two proteins in fibres; 81% MaSp1 and 19% MaSp2. Different spiders have different ratios of MaSp1 and MaSp2. For example, a dragline silk fibre from the orb weaver Argiope aurantia contains 41% MaSp1 and 59% MaSp2. Such changes in the ratios of major ampullate silks can dictate the performance of the silk fibre.

At least seven different types of silk proteins are known for one orb-weaver species of spider. Silks differ in primary sequence, physical properties and functions. For example, dragline silks used to build frames, radii and lifelines are known for outstanding mechanical properties including strength, toughness and elasticity. On an equal weight basis, spider silk has a higher toughness than steel and Kevlar. Flageliform silk found in capture spirals has extensibility of up to 500%. Minor ampullate silk, which is found in auxiliary spirals of the orb-web and in prey wrapping, possesses high toughness and strength almost similar to major ampullate silks, but does not supercontract in water.

Spider silks are known for their high tensile strength and toughness. The recombinant silk proteins also confer advantageous properties to cosmetic or dermatological compositions, in particular to be able to improve the hydrating or softening action, good film forming property and low surface density. Diverse and unique biomechanical properties together with biocompatibility and a slow rate of degradation make spider silks excellent candidates as biomaterials for tissue engineering, guided tissue repair and drug delivery, for cosmetic products (e.g. nail and hair strengthener, skin care products), and industrial materials (e.g. nanowires, nanofibers, surface coatings).

In an embodiment, a silk protein may include a polypeptide derived from natural spider silk proteins. The polypeptide is not limited particularly as long as it is derived from natural spider silk proteins, and examples of the polypeptide include natural spider silk proteins and recombinant spider silk proteins such as variants, analogs, derivatives or the like of the natural spider silk proteins. In terms of excellent tenacity, the polypeptide may be derived from major dragline silk proteins produced in major ampullate glands of spiders. Examples of the major dragline silk proteins include major ampullate spidroin MaSp1 and MaSp2 from Nephila clavipes, and ADF3 and ADF4 from Araneus diadematus, etc. Examples of the polypeptide derived from major dragline silk proteins include variants, analogs, derivatives or the like of the major dragline silk proteins. Further, the polypeptide may be derived from flagelliform silk proteins produced in flagelliform glands of spiders. Examples of the flagelliform silk proteins include flagelliform silk proteins derived from Nephila clavipes, etc.

Examples of the polypeptide derived from major dragline silk proteins include a polypeptide containing two or more units of an amino acid sequence represented by the formula 1: REP1-REP2 (1), preferably a polypeptide containing five or more units thereof, and more preferably a polypeptide containing ten or more units thereof. Alternatively, the polypeptide derived from major dragline silk proteins may be a polypeptide that contains units of the amino acid sequence represented by the formula 1: REP1-REP2 (1) and that has, at a C-terminal, an amino acid sequence represented by any of SEQ ID NOS: 52 to 54, which is also described in U.S. Pat. No. 9,051,453, which is incorporated by reference herein in its entirety, or an amino acid sequence having a homology of 90% or more with the amino acid sequence represented by any of SEQ ID NOS: 52 to 54, which is also described in U.S. Pat. No. 9,051,453, which is incorporated by reference herein in its entirety. In the polypeptide derived from major dragline silk proteins, units of the amino acid sequence represented by the formula 1: REP1-REP2 (1) may be the same or may be different from each other. In the case of producing a recombinant protein using a microbe such as Escherichia coli as a host, the molecular weight of the polypeptide derived from major dragline silk proteins is 500 kDa or less, or 300 kDa or less, or 200 kDa or less, in terms of productivity.

In the formula (1), the REP1 indicates polyalanine. In the REP1, the number of alanine residues arranged in succession is preferably 2 or more, more preferably 3 or more, further preferably 4 or more, and particularly preferably 5 or more. Further, in the REP1, the number of alanine residues arranged in succession is preferably 20 or less, more preferably 16 or less, further preferably 12 or less, and particularly preferably 10 or less. In the formula (1), the REP2 is an amino acid sequence composed of 10 to 200 amino acid residues. The total number of glycine, serine, glutamine and alanine residues contained in the amino acid sequence is 40% or more, preferably 60% or more, and more preferably 70% or more with respect to the total number of amino acid residues contained therein.

In the major dragline silk, the REP1 corresponds to a crystal region in a fiber where a crystal β sheet is formed, and the REP2 corresponds to an amorphous region in a fiber where most of the parts lack regular configurations and that has more flexibility. Further, the [REP1-REP2] corresponds to a repetitious region (repetitive sequence) composed of the crystal region and the amorphous region, which is a characteristic sequence of dragline silk proteins.

Recombinant Silk Fragments

In some embodiments, the recombinant silk protein refers to recombinant spider silk polypeptides, recombinant insect silk polypeptides, or recombinant mussel silk polypeptides. In some embodiments, the recombinant silk protein fragment disclosed herein include recombinant spider silk polypeptides of Araneidae or Araneoids, or recombinant insect silk polypeptides of Bombyx mori. In some embodiments, the recombinant silk protein fragment disclosed herein include recombinant spider silk polypeptides of Araneidae or Araneoids. In some embodiments, the recombinant silk protein fragment disclosed herein include block copolymer having repetitive units derived from natural spider silk polypeptides of Araneidae or Araneoids. In some embodiments, the recombinant silk protein fragment disclosed herein include block copolymer having synthetic repetitive units derived from spider silk polypeptides of Araneidae or Araneoids and non-repetitive units derived from natural repetitive units of spider silk polypeptides of Araneidae or Araneoids.

Recent advances in genetic engineering have provided a route to produce various types of recombinant silk proteins. Recombinant DNA technology has been used to provide a more practical source of silk proteins. As used herein “recombinant silk protein” refers to synthetic proteins produced heterologously in prokaryotic or eukaryotic expression systems using genetic engineering methods.

Various methods for synthesizing recombinant silk peptides are known and have been described by Ausubel et al., Current Protocols in Molecular Biology § 8 (John Wiley & Sons 1987, (1990)), incorporated herein by reference. A gram-negative, rod-shaped bacterium E. coli is a well-established host for industrial scale production of proteins. Therefore, the majority of recombinant silks have been produced in E. coli. E. coli which is easy to manipulate, has a short generation time, is relatively low cost and can be scaled up for larger amounts protein production.

The recombinant silk proteins can be produced by transformed prokaryotic or eukaryotic systems containing the cDNA coding for a silk protein, for a fragment of this protein or for an analog of such a protein. The recombinant DNA approach enables the production of recombinant silks with programmed sequences, secondary structures, architectures and precise molecular weight. There are four main steps in the process: (i) design and assembly of synthetic silk-like genes into genetic ‘cassettes’, (ii) insertion of this segment into a DNA recombinant vector, (iii) transformation of this recombinant DNA molecule into a host cell and (iv) expression and purification of the selected clones.

The term “recombinant vectors”, as used herein, includes any vectors known to the skilled person including plasmid vectors, cosmid vectors, phage vectors such as lambda phage, viral vectors such as adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, or plant) or in in vitro expression systems. Cloning vectors are generally used to engineer and amplify a certain desired DNA fragment and may lack functional sequences needed for expression of the desired DNA fragments.

The prokaryotic systems include Gram-negative bacteria or Gram-positive bacteria. The prokaryotic expression vectors can include an origin of replication which can be recognized by the host organism, a homologous or heterologous promoter which is functional in the said host, the DNA sequence coding for the spider silk protein, for a fragment of this protein or for an analogous protein. Nonlimiting examples of prokaryotic expression organisms are Escherichia coli, Bacillus subtilis, Bacillus megaterium, Corynebacterium glutamicum, Anabaena, Caulobacter, Gluconobacter, Rhodobacter, Pseudomonas, Para coccus, Bacillus (e.g. Bacillus subtilis) Brevibacterium, Corynebacterium, Rhizobium (Sinorhizobium), Flavobacterium, Klebsiella, Enterobacter, Lactobacillus, Lactococcus, Methylobacterium, Propionibacterium, Staphylococcus or Streptomyces cells.

The eukaryotic systems include yeasts and insect, mammalian or plant cells. In this case, the expression vectors can include a yeast plasmid origin of replication or an autonomous replication sequence, a promoter, a DNA sequence coding for a spider silk protein, for a fragment or for an analogous protein, a polyadenylation sequence, a transcription termination site and, lastly, a selection gene. Nonlimiting examples of eukaryotic expression organisms include yeasts, such as Saccharomyces cerevisiae, Pichia pastoris, basidiosporogenous, ascosporogenous, filamentous fungi, such as Aspergillus niger, Aspergillus oryzae, Aspergillus nidulans, Trichoderma reesei, Acremonium chrysogenum, Candida, Hansenula, Kluyveromyces, Saccharomyces (e.g. Saccharomyces cerevisiae), Schizosaccharomyces, Pichia (e.g. Pichia pastoris) or Yarrowia cells etc., mammalian cells, such as HeLa cells, COS cells, CHO cells etc., insect cells, such as Sf9 cells, MEL cells, etc., “insect host cells” such as Spodoptera frugiperda or Trichoplusia ni cells. SF9 cells, SF-21 cells or High-Five cells, wherein SF-9 and SF-21 are ovarian cells from Spodoptera frugiperda, and High-Five cells are egg cells from Trichoplusia ni., “plant host cells”, such as tobacco, potato or pea cells.

A variety of heterologous host systems have been explored to produce different types of recombinant silks. Recombinant partial spidroins as well as engineered silks have been cloned and expressed in bacteria (Escherichia coli), yeast (Pichia pastoris), insects (silkworm larvae), plants (tobacco, soybean, potato, Arabidopsis), mammalian cell lines (BHT/hamster) and transgenic animals (mice, goats). Most of the silk proteins are produced with an N- or C-terminal His-tags to make purification simple and produce enough amounts of the protein.

In some embodiments, the host suitable for expressing the recombinant spider silk protein using heterogeneous system may include transgenic animals and plants. In some embodiments, the host suitable for expressing the recombinant spider silk protein using heterogeneous system comprises bacteria, yeasts, mammalian cell lines. In some embodiments, the host suitable for expressing the recombinant spider silk protein using heterogeneous system comprises E. coli. In some embodiments, the host suitable for expressing the recombinant spider silk protein using heterogeneous system comprises transgenic B. mori silkworm generated using genome editing technologies (e.g. CRISPR).

The recombinant silk protein in this disclosure comprises synthetic proteins which are based on repeat units of natural silk proteins. Besides the synthetic repetitive silk protein sequences, these can additionally comprise one or more natural nonrepetitive silk protein sequences.

In some embodiments, “recombinant silk protein” refers to recombinant silkworm silk protein or fragments thereof. The recombinant production of silk fibroin and silk sericin has been reported. A variety of hosts are used for the production including E. coli, Saccharomyces cerevisiae, Pseudomonas sp., Rhodopseudomonas sp., Bacillus sp., and Streptomyces. See EP 0230702, which is incorporate by reference herein by its entirety.

Provided herein also include design and biological-synthesis of silk fibroin protein-like multiblock polymer comprising GAGAGX (SEQ ID NO: 1) hexapeptide (X is A, Y, V or S) derived from the repetitive domain of B. mori silk heavy chain (H chain)

In some embodiments, this disclosure provides silk protein-like multiblock polymers derived from the repetitive domain of B. mori silk heavy chain (H chain) comprising the GAGAGS (SEQ ID NO: 2) hexapeptide repeating units. The GAGAGS (SEQ ID NO: 2) hexapeptide is the core unit of H-chain and plays an important role in the formation of crystalline domains. The silk protein-like multiblock polymers containing the GAGAGS (SEQ ID NO: 2) hexapeptide repeating units spontaneously aggregate into β-sheet structures, similar to natural silk fibroin protein, where in the silk protein-like multiblock polymers having any weight average molecular weight described herein.

In some embodiments, this disclosure provides silk-peptide like multiblock copolymers composed of the GAGAGS (SEQ ID NO: 2) hexapeptide repetitive fragment derived from H chain of B. mori silk heavy chain and mammalian elastin VPGVG (SEQ ID NO: 3) motif produced by E. coli. In some embodiments, this disclosure provides fusion silk fibroin proteins composed of the GAGAGS (SEQ ID NO: 2) hexapeptide repetitive fragment derived from H chain of B. mori silk heavy chain and GVGVP (SEQ ID NO: 4) produced by E. coli, where in the silk protein-like multiblock polymers having any weight average molecular weight described herein.

In some embodiments, this disclosure provides B. mori silkworm recombinant proteins composed of the (GAGAGS)₁₆ (SEQ ID NO: 55) repetitive fragment. In some embodiments, this disclosure provides recombinant proteins composed of the (GAGAGS)₁₆ (SEQ ID NO: 55) repetitive fragment and the non-repetitive (GAGAGS)₁₆—F—COOH (SEQ ID NO: 56), (GAGAGS)₁₆—F—F—COOH (SEQ ID NO: 57), (GAGAGS)₁₆—F—F—F—COOH (SEQ ID NO: 58), (GAGAGS)₁₆—F—F—F—F—COOH (SEQ ID NO: 59), (GAGAGS)₁₆—F—F—F—F—F—F—F—F—COOH (SEQ ID NO: 60), (GAGAGS)₁₆—F—F—F—F—F—F—F—F—F—F—F—F—COOH (SEQ ID NO: 61) produced by E. coli, where F has the following amino acid sequence SGFGPVANGGSGEASSESDFGSSGFGPVANASSGEASSESDFAG (SEQ ID NO: 5), and where in the silk protein-like multiblock polymers having any weight average molecular weight described herein.

In some embodiments, “recombinant silk protein” refers to recombinant spider silk protein or fragments thereof. The productions of recombinant spider silk proteins based on a partial cDNA clone have been reported. The recombinant spider silk proteins produced as such comprise a portion of the repetitive sequence derived from a dragline spider silk protein, Spidroin 1, from the spider Nephila clavipes. see Xu et al. (Proc. Natl. Acad. Sci. U.S.A., 87:7120-7124 (1990). cDNA clone encoding a portion of the repeating sequence of a second fibroin protein, Spidroin 2, from dragline silk of Nephila clavipes and the recombinant synthesis thereof is described in J. Biol. Chem., 1992, volume 267, pp. 19320-19324. The recombinant synthesis of spider silk proteins including protein fragments and variants of Nephila clavipes from transformed E. coli is described in U.S. Pat. Nos. 5,728,810 and 5,989,894. cDNA clones encoding minor ampullate spider silk proteins and the expression thereof is described in U.S. Pat. Nos. 5,733,771 and 5,756,677. cDNA clone encoding the flagelliform silk protein from an orb-web spinning spider is described in U.S. Pat. No. 5,994,099. U.S. Pat. No. 6,268,169 describes the recombinant synthesis of spider silk like proteins derived from the repeating peptide sequence found in the natural spider dragline of Nephila clavipes by E. coli, Bacillus subtilis, and Pichia pastoris recombinant expression systems. WO 03/020916 describes the cDNA clone encoding and recombinant production of spider silk proteins having repetitive sequences derived from the major ampullate glands of Nephila madagascariensis, Nephila senegalensis, Tetragnatha kauaiensis, Tetragnatha versicolor, Argiope aurantia, Argiope trifasciata, Gasteracantha mammosa, and Latrodectus geometricus, the flagelliform glands of Argiope trifasciata, the ampullate glands of Dolomedes tenebrosus, two sets of silk glands from Plectreurys tristis, and the silk glands of the mygalomorph Euagrus chisoseus. Each of the above reference is incorporated herein by reference in its entirety.

In some embodiments, the recombinant spider silk protein is a hybrid protein of a spider silk protein and an insect silk protein, a spider silk protein and collagen, a spider silk protein and resilin, or a spider silk protein and keratin. The spider silk repetitive unit comprises or consists of an amino acid sequence of a region that comprises or consists of at least one peptide motif that repetitively occurs within a naturally occurring major ampullate gland polypeptide, such as a dragline spider silk polypeptide, a minor ampullate gland polypeptide, a flagelliform polypeptide, an aggregate spider silk polypeptide, an aciniform spider silk polypeptide or a pyriform spider silk polypeptide.

In some embodiments, the recombinant spider silk protein in this disclosure comprises synthetic spider silk proteins derived from repetitive units of natural spider silk proteins, consensus sequence, and optionally one or more natural non-repetitive spider silk protein sequences. The repeated units of natural spider silk polypeptide may include dragline spider silk polypeptides or flagelliform spider silk polypeptides of Araneidae or Araneoids.

As used herein, the spider silk “repetitive unit” comprises or consists of at least one peptide motif that repetitively occurs within a naturally occurring major ampullate gland polypeptide, such as a dragline spider silk polypeptide, a minor ampullate gland polypeptide, a flagelliform polypeptide, an aggregate spider silk polypeptide, an aciniform spider silk polypeptide or a pyriform spider silk polypeptide. A “repetitive unit” refers to a region which corresponds in amino acid sequence to a region that comprises or consists of at least one peptide motif (e.g. AAAAAA (SEQ ID NO: 20)) or GPGQQ (SEQ ID NO: 15)) that repetitively occurs within a naturally occurring silk polypeptide (e.g. MaSpI, ADF-3, ADF-4, or Flag) (i.e. identical amino acid sequence) or to an amino acid sequence substantially similar thereto (i.e. variational amino acid sequence). A “repetitive unit” having an amino acid sequence which is “substantially similar” to a corresponding amino acid sequence within a naturally occurring silk polypeptide (i.e. wild-type repetitive unit) is also similar with respect to its properties, e.g. a silk protein comprising the “substantially similar repetitive unit” is still insoluble and retains its insolubility. A “repetitive unit” having an amino acid sequence which is “identical” to the amino acid sequence of a naturally occurring silk polypeptide, for example, can be a portion of a silk polypeptide corresponding to one or more peptide motifs of MaSpI (SEQ ID NO: 48), MaSpII (SEQ ID NO: 49), ADF-3 (SEQ ID NO: 50) and/or ADF-4 (SEQ ID NO: 51). A “repetitive unit” having an amino acid sequence which is “substantially similar” to the amino acid sequence of a naturally occurring silk polypeptide, for example, can be a portion of a silk polypeptide corresponding to one or more peptide motifs of MaSpI (SEQ ID NO: 48), MaSpII (SEQ ID NO: 49), ADF-3 (SEQ ID NO: 50) and/or ADF-4 (SEQ ID NO: 51) but having one or more amino acid substitution at specific amino acid positions.

As used herein, the term “consensus peptide sequence” refers to an amino acid sequence which contains amino acids which frequently occur in a certain position (e.g. “G”) and wherein, other amino acids which are not further determined are replaced by the place holder “X”. In some embodiments, the consensus sequence is at least one of (i) GPGXX (SEQ ID NO: 6), wherein X is an amino acid selected from A, S, G, Y, P and Q; (ii) GGX, wherein X is an amino acid selected from Y, P, R, S, A, T, N and Q, preferably Y, P and Q; (iii) Aₓ, wherein x is an integer from 5 to 10.

The consensus peptide sequences GPGXX (SEQ ID NO: 6) and GGX, i.e. glycine rich motifs, provide flexibility to the silk polypeptide and thus, to the thread formed from the silk protein containing said motifs. In detail, the iterated GPGXX (SEQ ID NO: 6) motif forms turn spiral structures, which imparts elasticity to the silk polypeptide. Major ampullate and flagelliform silks both have a GPGXX (SEQ ID NO: 6) motif. The iterated GGX motif is associated with a helical structure having three amino acids per turn and is found in most spider silks. The GGX motif may provide additional elastic properties to the silk. The iterated polyalanine Ax (peptide) motif forms a crystalline β-sheet structure that provides strength to the silk polypeptide, as described for example in WO 03/057727.

In some embodiments, the recombinant spider silk protein in this disclosure comprises two identical repetitive units each comprising at least one, preferably one, amino acid sequence selected from the group consisting of: GGRPSDTYG (SEQ ID NO: 7) and GGRPSSSYG (SEQ ID NO: 8) derived from Resilin. Resilin is an elastomeric protein found in most arthropods that provides low stiffness and high strength.

As used herein, “non-repetitive units” refers to an amino acid sequence which is “substantially similar” to a corresponding non-repetitive (carboxy terminal) amino acid sequence within a naturally occurring dragline polypeptide (i.e. wild-type non-repetitive (carboxy terminal) unit), preferably within ADF-3 (SEQ ID NO: 50), ADF-4 (SEQ ID NO: 51), NR3 (SEQ ID NO: 62), NR4 (SEQ ID NO: 63) of the spider Araneus diadematus, which is also described in U.S. Pat. No. 9,217,017, which is incorporated by reference herein in its entirety, C16 peptide (spider silk protein eADF4, molecular weight of 47.7 kDa, AMSilk) comprising the 16 repeats of the sequence GSSAAAAAAAASGPGGYGPENQGPSGPGGYGPGGP (SEQ ID NO: 9), an amino acid sequence adapted from the natural sequence of ADF4 from A. diadematus. Non-repetitive ADF-4 and variants thereof display efficient assembly behavior.

Among the synthetic spider silk proteins, the recombinant silk protein in this disclosure comprises in some embodiments the C16-protein having the polypeptide sequence SEQ ID NO: 64, which is also described in U.S. Pat. No. 8,288,512, which is incorporated by reference herein in its entirety. Besides the polypeptide sequence shown in SEQ ID NO: 64, particularly functional equivalents, functional derivatives and salts of this sequence are also included.

As used herein, “functional equivalents” refers to mutant which, in at least one sequence position of the abovementioned amino acid sequences, have an amino acid other than that specifically mentioned.

In some embodiments, the recombinant spider silk protein in this disclosure comprises, in an effective amount, at least one natural or recombinant silk protein including spider silk protein, corresponding to Spidroin major 1 described by Xu et al., PNAS, USA, 87, 7120, (1990), Spidroin major 2 described by Hinman and Lewis, J. Biol. Chem., 267, 19320, (1922), recombinant spider silk protein as described in U.S. Patent Application No. 2016/0222174 and U.S. Pat. Nos. 9,051,453, 9,617,315, 9,689,089, 8,173,772, 8,642,734, 8,367,803 8,097,583, 8,030,024, 7,754,851, 7,148,039, 7,060,260, or alternatively the minor Spidroins described in patent application WO 95/25165. Each of the above-cited references is incorporated herein by reference in its entirety. Additional recombinant spider silk proteins suitable for the recombinant RSPF of this disclosure include ADF3 and ADF4 from the “Major Ampullate” gland of Araneus diadematus.

Recombinant silk is also described in other patents and patent applications, incorporated by reference herein: US 2004590196, U.S. Pat. No. 7,754,851, US 2007654470, U.S. Pat. No. 7,951,908, US 2010785960, U.S. Pat. No. 8,034,897, US 20090263430, US 2008226854, US 20090123967, US 2005712095, US 2007991037, US 20090162896, US 200885266, U.S. Pat. No. 8,372,436, US 2007989907, US 2009267596, US 2010319542, US 2009265344, US 2012684607, US 2004583227, U.S. Pat. No. 8,030,024, US 2006643569, U.S. Pat. No. 7,868,146, US 2007991916, U.S. Pat. No. 8,097,583, US 2006643200, U.S. Pat. Nos. 8,729,238, 8,877,903, US 20190062557, US 20160280960, US 20110201783, US 2008991916, US 2011986662, US 2012697729, US 20150328363, U.S. Pat. No. 9,034,816, US 20130172478, U.S. Pat. No. 9,217,017, US 20170202995, U.S. Pat. No. 8,721,991, US 2008227498, U.S. Pat. Nos. 9,233,067, 8,288,512, US 2008161364, U.S. Pat. No. 7,148,039, U.S. Ser. No. 19/992,47806, US 2001861597, US 2004887100, U.S. Pat. Nos. 9,481,719, 8,765,688, US 200880705, US 2010809102, U.S. Pat. No. 8,367,803, US 2010664902, U.S. Pat. No. 7,569,660, U.S. Ser. No. 19/991,38833, US 2000591632, US 20120065126, US 20100278882, US 2008161352, US 20100015070, US 2009513709, US 20090194317, US 2004559286, US 200589551, US 2008187824, US 20050266242, US 20050227322, and US 20044418.

Recombinant silk is also described in other patents and patent applications, incorporated by reference herein: US 20190062557, US 20150284565, US 20130225476, US 20130172478, US 20130136779, US 20130109762, US 20120252294, US 20110230911, US 20110201783, US 20100298877, U.S. Pat. Nos. 10,478,520, 10,253,213, 10,072,152, 9,233,067, 9,217,017, 9,034,816, 8,877,903, 8,729,238, 8,721,991, 8,097,583, 8,034,897, 8,030,024, 7,951,908, 7,868,146, and 7,754,851.

In some embodiments, the recombinant spider silk protein in this disclosure comprises or consists of 2 to 80 repetitive units, each independently selected from GPGXX (SEQ ID NO: 6), GGX and Aₓ as defined herein.

In some embodiments, the recombinant spider silk protein in this disclosure comprises or consists of repetitive units each independently selected from selected from the group consisting of GPGAS (SEQ ID NO: 10), GPGSG (SEQ ID NO: 11), GPGGY (SEQ ID NO: 12), GPGGP (SEQ ID NO: 13), GPGGA (SEQ ID NO: 14), GPGQQ (SEQ ID NO: 15), GPGGG (SEQ ID NO: 16), GPGQG (SEQ ID NO: 17), GPGGS (SEQ ID NO: 18), GGY, GGP, GGA, GGR, GGS, GGT, GGN, GGQ, AAAAA (SEQ ID NO: 19), AAAAAA (SEQ ID NO: 20), AAAAAAA (SEQ ID NO: 21), AAAAAAAA (SEQ ID NO: 22), AAAAAAAAA (SEQ ID NO: 23), AAAAAAAAAA (SEQ ID NO: 24), GGRPSDTYG (SEQ ID NO: 7) and GGRPSSSYG (SEQ ID NO: 8), (i) GPYGPGASAAAAAAGGYGPGSGQQ (SEQ ID NO: 25), (ii) GSSAAAAAAAASGPGGYGPENQGPSGPGGYGPGGP (SEQ ID NO: 9), (iii) GPGQQGPGQQGPGQQGPGQQ (SEQ ID NO: 26): (iv) GPGGAGGPYGPGGAGGPYGPGGAGGPY (SEQ ID NO: 27), (v) GGTTIIEDLDITIDGADGPITISEELTI (SEQ ID NO: 28), (vi) PGSSAAAAAAAASGPGQGQGQGQGQGGRPSDTYG (SEQ ID NO: 29), (vii) SAAAAAAAAGPGGGNGGRPSDTYGAPGGGNGGRPSSSYG (SEQ ID NO: 30), (viii) GGAGGAGGAGGSGGAGGS (SEQ ID NO: 31), (ix) GPGGAGPGGYGPGGSGPGGYGPGGSGPGGY (SEQ ID NO: 32), (x) GPYGPGASAAAAAAGGYGPGCGQQ (SEQ ID NO: 33), (xi) GPYGPGASAAAAAAGGYGPGKGQQ (SEQ ID NO: 34), (xii) GSSAAAAAAAASGPGGYGPENQGPCGPGGYGPGGP (SEQ ID NO: 35), (xiii) GSSAAAAAAAASGPGGYGPKNQGPSGPGGYGPGGP (SEQ ID NO: 36), (xiv) GSSAAAAAAAASGPGGYGPKNQGPCGPGGYGPGGP (SEQ ID NO: 37), or variants thereof as described in U.S. Pat. No. 8,877,903, for example, a synthetic spider peptide having sequential order of GPGAS (SEQ ID NO: 10), GGY, GPGSG (SEQ ID NO: 11) in the peptide chain, or sequential order of AAAAAAAA (SEQ ID NO: 22), GPGGY (SEQ ID NO: 12), GPGGP (SEQ ID NO: 13) in the peptide chain, sequential order of AAAAAAAA (SEQ ID NO: 22), GPGQG (SEQ ID NO: 17), GGR in the peptide chain.

In some embodiments, this disclosure provides silk protein-like multiblock peptides that imitate the repeating units of amino acids derived from natural spider silk proteins such as Spidroin major 1 domain, Spidroin major 2 domain or Spidroin minor 1 domain and the profile of variation between the repeating units without modifying their three-dimensional conformation, wherein these silk protein-like multiblock peptides comprise a repeating unit of amino acids corresponding to one of the sequences (I), (II), (III) and/or (IV) below.

[(XGG)_{w}(XGA)(GXG)ₓ(AGA)_{y}(G)_{z}AG]ₚ (SEQ ID NO: 38) Formula (I) in which: X corresponds to tyrosine or to glutamine, w is an integer equal to 2 or 3, x is an integer from 1 to 3, y is an integer from 5 to 7, z is an integer equal to 1 or 2, and p is an integer and having any weight average molecular weight described herein, and/or

[(GPG₂YGPGQ₂)ₐ(X′)₂S(A)_{b}]ₚ (SEQ ID NO: 39) Formula (II) in which: X′ corresponds to the amino acid sequence GPS or GPG, a is equal to 2 or 3, b is an integer from 7 to 10, and p is an integer and having any weight average molecular weight described herein, and/or

[(GR)(GA)ₗ(A)ₘ(GGX)ₙ(GA)ₗ(A)ₘ]ₚ (SEQ ID NO: 40) Formula (III) and/or [(GGX″)ₙ(GA)ₘ(A)ₗ]ₚ (SEQ ID NO: 41) Formula (IV) in which: X″ corresponds to tyrosine, glutamine or alanine, l is an integer from 1 to 6, m is an integer from 0 to 4, n is an integer from 1 to 4, and p is an integer.

In some embodiments, the recombinant spider silk protein or an analog of a spider silk protein comprising an amino acid repeating unit of sequence (V):

[(Xaa Gly Gly)_{w}(Xaa Gly Ala)(Gly Xaa Gly)ₓ(Ala Gly Ala)_{y}(Gly)_{z}Ala Gly]ₚ Formula (V), wherein Xaa is tyrosine or glutamine, w is an integer equal to 2 or 3, x is an integer from 1 to 3, y is an integer from 5 to 7, z is an integer equal to 1 or 2, and p is an integer.

In some embodiments, the recombinant spider silk protein in this disclosure is selected from the group consisting of ADF-3 or variants thereof, ADF-4 or variants thereof, MaSpI or variants thereof, MaSpII or variants thereof as described in U.S. Pat. No. 9,217,017.

In some embodiments, this disclosure provides water soluble recombinant spider silk proteins produced in mammalian cells. The solubility of the spider silk proteins produced in mammalian cells was attributed to the presence of the COOH-terminus in these proteins, which makes them more hydrophilic. These COOH-terminal amino acids are absent in spider silk proteins expressed in microbial hosts.

In some embodiments, the recombinant spider silk protein in this disclosure comprises water soluble recombinant spider silk protein C16 modified with an amino or carboxyl terminal selected from the amino acid sequences consisting of: GCGGGGGG (SEQ ID NO: 42), GKGGGGGG (SEQ ID NO: 43), GCGGSGGGGSGGGG (SEQ ID NO: 44), GKGGGGGGSGGGG (SEQ ID NO: 45), and GCGGGGGGSGGGG (SEQ ID NO: 46). In some embodiments, the recombinant spider silk protein in this disclosure comprises C₁₆NR4, C₃₂NR4, C16, C32, NR4C₁₆NR4, NR4C₃₂NR4, NR3C₁₆NR3, or NR3C₃₂NR3 such that the molecular weight of the protein ranges as described herein.

In some embodiments, the recombinant spider silk protein in this disclosure comprises recombinant spider silk protein having a synthetic repetitive peptide segments and an amino acid sequence adapted from the natural sequence of ADF4 from A. diadematus as described in U.S. Pat. No. 8,877,903. In some embodiments, the RSPF in this disclosure comprises the recombinant spider silk proteins having repeating peptide units derived from natural spider silk proteins such as Spidroin major 1 domain, Spidroin major 2 domain or Spidroin minor 1 domain, wherein the repeating peptide sequence is GSSAAAAAAAASGPGQGQGQGQGQGGRPSDTYG (SEQ ID NO: 47) or SAAAAAAAAGPGGGNGGRPSDTYGAPGGGNGGRPSSSYG (SEQ ID NO: 30), as described in U.S. Pat. No. 8,367,803, which is incorporated by reference herein in its entirety.

In some embodiments, this disclosure provides recombinant spider proteins composed of the GPGGAGPGGYGPGGSGPGGYGPGGSGPGGY (SEQ ID NO: 32) repetitive fragment and having a molecular weight as described herein.

As used herein, the term “recombinant silk” refers to recombinant spider and/or silkworm silk protein or fragments thereof. In an embodiment, the spider silk protein is selected from the group consisting of swathing silk (Achniform gland silk), egg sac silk (Cylindriform gland silk), egg case silk (Tubuliform silk), non-sticky dragline silk (Ampullate gland silk), attaching thread silk (Pyriform gland silk), sticky silk core fibers (Flagelliform gland silk), and sticky silk outer fibers (Aggregate gland silk). For example, recombinant spider silk protein, as described herein, includes the proteins described in U.S. Patent Application No. 2016/0222174 and U.S. Pat. Nos. 9,051,453, 9,617,315, 9,689,089, 8,173,772, and 8,642,734.

Some organisms make multiple silk fibers with unique sequences, structural elements, and mechanical properties. For example, orb weaving spiders have six unique types of glands that produce different silk polypeptide sequences that are polymerized into fibers tailored to fit an environmental or lifecycle niche. The fibers are named for the gland they originate from and the polypeptides are labeled with the gland abbreviation (e.g. “Ma”) and “Sp” for spidroin (short for spider fibroin). In orb weavers, these types include Major Ampullate (MaSp, also called dragline), Minor Ampullate (MiSp), Flagelliform (Flag), Aciniform (AcSp), Tubuliform (TuSp), and Pyriform (PySp). This combination of polypeptide sequences across fiber types, domains, and variation amongst different genus and species of organisms leads to a vast array of potential properties that can be harnessed by commercial production of the recombinant fibers. To date, the vast majority of the work with recombinant silks has focused on the Major Ampullate Spidroins (MaSp).

Aciniform (AcSp) silks tend to have high toughness, a result of moderately high strength coupled with moderately high extensibility. AcSp silks are characterized by large block (“ensemble repeat”) sizes that often incorporate motifs of poly serine and GPX. Tubuliform (TuSp or Cylindrical) silks tend to have large diameters, with modest strength and high extensibility. TuSp silks are characterized by their poly serine and poly threonine content, and short tracts of poly alanine. Major Ampullate (MaSp) silks tend to have high strength and modest extensibility. MaSp silks can be one of two subtypes: MaSp1 and MaSp2. MaSp1 silks are generally less extensible than MaSp2 silks, and are characterized by poly alanine, GX, and GGX motifs. MaSp2 silks are characterized by poly alanine, GGX, and GPX motifs. Minor Ampullate (MiSp) silks tend to have modest strength and modest extensibility. MiSp silks are characterized by GGX, GA, and poly A motifs, and often contain spacer elements of approximately 100 amino acids. Flagelliform (Flag) silks tend to have very high extensibility and modest strength. Flag silks are usually characterized by GPG, GGX, and short spacer motifs.

Silk polypeptides are characteristically composed of a repeat domain (REP) flanked by non-repetitive regions (e.g., C-terminal and N-terminal domains). In an embodiment, both the C-terminal and N-terminal domains are between 75-350 amino acids in length. The repeat domain exhibits a hierarchical architecture. The repeat domain comprises a series of blocks (also called repeat units). The blocks are repeated, sometimes perfectly and sometimes imperfectly (making up a quasi-repeat domain), throughout the silk repeat domain. The length and composition of blocks varies among different silk types and across different species. Table 1 of U.S. Published Application No. 2016/0222174, the entirety of which is incorporated herein, lists examples of block sequences from selected species and silk types, with further examples presented in Rising, A. et al., Spider silk proteins: recent advances in recombinant production, structure-function relationships and biomedical applications, Cell Mol. Life Sci., 68:2, pg 169-184 (2011); and Gatesy, J. et al., Extreme diversity, conservation, and convergence of spider silk fibroin sequences, Science, 291:5513, pg. 2603-2605 (2001). In some cases, blocks may be arranged in a regular pattern, forming larger macro-repeats that appear multiple times (usually 2-8) in the repeat domain of the silk sequence. Repeated blocks inside a repeat domain or macro-repeat, and repeated macro-repeats within the repeat domain, may be separated by spacing elements.

The construction of certain spider silk block copolymer polypeptides from the blocks and/or macro-repeat domains, according to certain embodiments of the disclosure, is illustrated in U.S. Published Patent Application No. 2016/0222174.

The recombinant block copolymer polypeptides based on spider silk sequences produced by gene expression in a recombinant prokaryotic or eukaryotic system can be purified according to methods known in the art. In a preferred embodiment, a commercially available expression/secretion system can be used, whereby the recombinant polypeptide is expressed and thereafter secreted from the host cell, to be easily purified from the surrounding medium. If expression/secretion vectors are not used, an alternative approach involves purifying the recombinant block copolymer polypeptide from cell lysates (remains of cells following disruption of cellular integrity) derived from prokaryotic or eukaryotic cells in which a polypeptide was expressed. Methods for generation of such cell lysates are known to those of skill in the art. In some embodiments, recombinant block copolymer polypeptides are isolated from cell culture supernatant.

Recombinant block copolymer polypeptide may be purified by affinity separation, such as by immunological interaction with antibodies that bind specifically to the recombinant polypeptide or nickel columns for isolation of recombinant polypeptides tagged with 6-8 histidine residues at their N-terminus or C-terminus Alternative tags may comprise the FLAG epitope or the hemagglutinin epitope. Such methods are commonly used by skilled practitioners.

A solution of such polypeptides (i.e., recombinant silk protein) may then be prepared and used as described herein.

In another embodiment, recombinant silk protein may be prepared according to the methods described in U.S. Pat. No. 8,642,734, the entirety of which is incorporated herein, and used as described herein.

In an embodiment, a recombinant spider silk protein is provided. The spider silk protein typically consists of from 170 to 760 amino acid residues, such as from 170 to 600 amino acid residues, preferably from 280 to 600 amino acid residues, such as from 300 to 400 amino acid residues, more preferably from 340 to 380 amino acid residues. The small size is advantageous because longer spider silk proteins tend to form amorphous aggregates, which require use of harsh solvents for solubilization and polymerization. The recombinant spider silk protein may contain more than 760 residues, in particular in cases where the spider silk protein contains more than two fragments derived from the N-terminal part of a spider silk protein, The spider silk protein comprises an N-terminal fragment consisting of at least one fragment (NT) derived from the corresponding part of a spider silk protein, and a repetitive fragment (REP) derived from the corresponding internal fragment of a spider silk protein. Optionally, the spider silk protein comprises a C-terminal fragment (CT) derived from the corresponding fragment of a spider silk protein. The spider silk protein comprises typically a single fragment (NT) derived from the N-terminal part of a spider silk protein, but in preferred embodiments, the N-terminal fragment include at least two, such as two fragments (NT) derived from the N-terminal part of a spider silk protein. Thus, the spidroin can schematically be represented by the formula NTₘ-REP, and alternatively NTₘ-REP-CT, where m is an integer that is 1 or higher, such as 2 or higher, preferably in the ranges of 1-2, 1-4, 1-6, 2-4 or 2-6. Preferred spidroins can schematically be represented by the formulas NT₂-REP or NT-REP, and alternatively NT₂-REP-CT or NT-REP-CT. The protein fragments are covalently coupled, typically via a peptide bond. In one embodiment, the spider silk protein consists of the NT fragment(s) coupled to the REP fragment, which REP fragment is optionally coupled to the CT fragment.

In one embodiment, the first step of the method of producing polymers of an isolated spider silk protein involves expression of a polynucleic acid molecule which encodes the spider silk protein in a suitable host, such as Escherichia coli. The thus obtained protein is isolated using standard procedures. Optionally, lipopolysaccharides and other pyrogens are actively removed at this stage.

In the second step of the method of producing polymers of an isolated spider silk protein, a solution of the spider silk protein in a liquid medium is provided. By the terms “soluble” and “in solution” is meant that the protein is not visibly aggregated and does not precipitate from the solvent at 60,000×g. The liquid medium can be any suitable medium, such as an aqueous medium, preferably a physiological medium, typically a buffered aqueous medium, such as a 10-50 mM Tris-HCl buffer or phosphate buffer. The liquid medium has a pH of 6.4 or higher and/or an ion composition that prevents polymerization of the spider silk protein. That is, the liquid medium has either a pH of 6.4 or higher or an ion composition that prevents polymerization of the spider silk protein, or both.

Ion compositions that prevent polymerization of the spider silk protein can readily be prepared by the skilled person utilizing the methods disclosed herein. A preferred ion composition that prevents polymerization of the spider silk protein has an ionic strength of more than 300 mM. Specific examples of ion compositions that prevent polymerization of the spider silk protein include above 300 mM NaCl, 100 mM phosphate and combinations of these ions having desired preventive effect on the polymerization of the spider silk protein, e.g. a combination of 10 mM phosphate and 300 mM NaCl.

The presence of an NT fragment improves the stability of the solution and prevents polymer formation under these conditions. This can be advantageous when immediate polymerization may be undesirable, e.g. during protein purification, in preparation of large batches, or when other conditions need to be optimized. It is preferred that the pH of the liquid medium is adjusted to 6.7 or higher, such as 7.0 or higher, or even 8.0 or higher, such as up to 10.5, to achieve high solubility of the spider silk protein. It can also be advantageous that the pH of the liquid medium is adjusted to the range of 6.4-6.8, which provides sufficient solubility of the spider silk protein but facilitates subsequent pH adjustment to 6.3 or lower.

In the third step, the properties of the liquid medium are adjusted to a pH of 6.3 or lower and ion composition that allows polymerization. That is, if the liquid medium wherein the spider silk protein is dissolved has a pH of 6.4 or higher, the pH is decreased to 6.3 or lower. The skilled person is well aware of various ways of achieving this, typically involving addition of a strong or weak acid. If the liquid medium wherein the spider silk protein is dissolved has an ion composition that prevents polymerization, the ion composition is changed so as to allow polymerization. The skilled person is well aware of various ways of achieving this, e.g. dilution, dialysis or gel filtration. If required, this step involves both decreasing the pH of the liquid medium to 6.3 or lower and changing the ion composition so as to allow polymerization. It is preferred that the pH of the liquid medium is adjusted to 6.2 or lower, such as 6.0 or lower. In particular, it may be advantageous from a practical point of view to limit the pH drop from 6.4 or 6.4-6.8 in the preceding step to 6.3 or 6.0-6.3, e.g. 6.2 in this step. In a preferred embodiment, the pH of the liquid medium of this step is 3 or higher, such as 4.2 or higher. The resulting pH range, e.g. 4.2-6.3 promotes rapid polymerization,

In the fourth step, the spider silk protein is allowed to polymerize in the liquid medium having pH of 6.3 or lower and an ion composition that allows polymerization of the spider silk protein. Although the presence of the NT fragment improves solubility of the spider silk protein at a pH of 6.4 or higher and/or an ion composition that prevents polymerization of the spider silk protein, it accelerates polymer formation at a pH of 6.3 or lower when the ion composition allows polymerization of the spider silk protein. The resulting polymers are preferably solid and macroscopic, and they are formed in the liquid medium having a pH of 6.3 or lower and an ion composition that allows polymerization of the spider silk protein. In a preferred embodiment, the pH of the liquid medium of this step is 3 or higher, such as 4.2 or higher. The resulting pH range, e.g. 4.2-6.3 promotes rapid polymerization, Resulting polymer may be provided at the molecular weights described herein and prepared as a solution form that may be used as necessary for article coatings.

Ion compositions that allow polymerization of the spider silk protein can readily be prepared by the skilled person utilizing the methods disclosed herein. A preferred ion composition that allows polymerization of the spider silk protein has an ionic strength of less than 300 mM. Specific examples of ion compositions that allow polymerization of the spider silk protein include 150 mM NaCl, 10 mM phosphate, 20 mM phosphate and combinations of these ions lacking preventive effect on the polymerization of the spider silk protein, e.g. a combination of 10 mM phosphate or 20 mM phosphate and 150 mM NaCl. It is preferred that the ionic strength of this liquid medium is adjusted to the range of 1-250 mM.

Without desiring to be limited to any specific theory, it is envisaged that the NT fragments have oppositely charged poles, and that environmental changes in pH affects the charge balance on the surface of the protein followed by polymerization, whereas salt inhibits the same event.

At neutral pH, the energetic cost of burying the excess negative charge of the acidic pole may be expected to prevent polymerization. However, as the dimer approaches its isoelectric point at lower pH, attractive electrostatic forces will eventually become dominant, explaining the observed salt and pH-dependent polymerization behavior of NT and NT-containing minispidroins. It is proposed that, in some embodiments, pH-induced NT polymerization, and increased efficiency of fiber assembly of NT-minispidroins, are due to surface electrostatic potential changes, and that clustering of acidic residues at one pole of NT shifts its charge balance such that the polymerization transition occurs at pH values of 6.3 or lower.

In a fifth step, the resulting, preferably solid spider silk protein polymers are isolated from said liquid medium. Optionally, this step involves actively removing lipopolysaccharides and other pyrogens from the spidroin polymers.

Without desiring to be limited to any specific theory, it has been observed that formation of spidroin polymers progresses via formation of water-soluble spidroin dimers. The present disclosure thus also provides a method of producing dimers of an isolated spider silk protein, wherein the first two method steps are as described above. The spider silk proteins are present as dimers in a liquid medium at a pH of 6.4 or higher and/or an ion composition that prevents polymerization of said spider silk protein. The third step involves isolating the dimers obtained in the second step, and optionally removal of lipopolysaccharides and other pyrogens. In a preferred embodiment, the spider silk protein polymer of the disclosure consists of polymerized protein dimers. The present disclosure thus provides a novel use of a spider silk protein, preferably those disclosed herein, for producing dimers of the spider silk protein.

According to another aspect, the disclosure provides a polymer of a spider silk protein as disclosed herein. In an embodiment, the polymer of this protein is obtainable by any one of the methods therefor according to the disclosure. Thus, the disclosure provides various uses of recombinant spider silk protein, preferably those disclosed herein, for producing polymers of the spider silk protein as recombinant silk based coatings. According to one embodiment, the present disclosure provides a novel use of a dimer of a spider silk protein, preferably those disclosed herein, for producing polymers of the isolated spider silk protein as recombinant silk based coatings. In these uses, it is preferred that the polymers are produced in a liquid medium having a pH of 6.3 or lower and an ion composition that allows polymerization of said spider silk protein. In an embodiment, the pH of the liquid medium is 3 or higher, such as 4.2 or higher. The resulting pH range, e.g. 4.2-6.3 promotes rapid polymerization,

Using the method(s) of the present disclosure, it is possible to control the polymerization process, and this allows for optimization of parameters for obtaining silk polymers with desirable properties and shapes.

In an embodiment, the recombinant silk proteins described herein, include those described in U.S. Pat. No. 8,642,734, the entirety of which is incorporated by reference.

In another embodiment, the recombinant silk proteins described herein may be prepared according to the methods described in U.S. Pat. No. 9,051,453, the entirety of which is incorporated herein by reference.

An amino acid sequence represented by SEQ ID NO: 52, which is also described in U.S. Pat. No. 9,051,453, is identical to an amino acid sequence that is composed of 50 amino acid residues of an amino acid sequence of ADF3 at the C-terminal (NCBI Accession No.: AAC47010, GI: 1263287). An amino acid sequence represented by SEQ ID NO: 53, which is also described in U.S. Pat. No. 9,051,453, is identical to an amino acid sequence represented by SEQ ID NO: 52, which is also described in U.S. Pat. No. 9,051,453, from which 20 residues have been removed from the C-terminal. An amino acid sequence represented by SEQ ID NO: 54, which is also described in U.S. Pat. No. 9,051,453, is identical to an amino acid sequence represented by SEQ ID NO: 52 from which 29 residues have been removed from the C-terminal.

An example of the polypeptide that contains units of the amino acid sequence represented by the formula 1: REP1-REP2 (1) and that has, at a C-terminal, an amino acid sequence represented by any of SEQ ID NOS: 52 to 54 or an amino acid sequence having a homology of 90% or more with the amino acid sequence represented by any of SEQ ID NOS: 52 to 54, which are also described in U.S. Pat. No. 9,051,453, is a polypeptide having an amino acid sequence represented by SEQ ID NO: 65, which is also described in U.S. Pat. No. 9,051,453, which is incorporated by reference herein in its entirety. The polypeptide having the amino acid sequence represented by SEQ ID NO: 65, which is also described in U.S. Pat. No. 9,051,453, is obtained by the following mutation: in an amino acid sequence of ADF3 (NCBI Accession No.: AAC47010, GI: 1263287) to the N-terminal of which has been added an amino acid sequence (SEQ ID NO: 66, which is also described in U.S. Pat. No. 9,051,453) composed of a start codon, His 10 tags and an HRV3C Protease (Human rhinovirus 3C Protease) recognition site, 1^{st} to 13^{th} repetitive regions are about doubled and the translation ends at the 1154^{th} amino acid residue. In the polypeptide having the amino acid sequence represented by SEQ ID NO: 65, which is also described in U.S. Pat. No. 9,051,453, the C-terminal sequence is identical to the amino acid sequence represented by SEQ ID NO: 54.

Further, the polypeptide that contains units of the amino acid sequence represented by the formula 1: REP1-REP2 (1) and that has, at a C-terminal, an amino acid sequence represented by any of SEQ ID NOS: 52 to 54, which are also described in U.S. Pat. No. 9,051,453, or an amino acid sequence having a homology of 90% or more with the amino acid sequence represented by any of SEQ ID NOS: 52 to 54, which are also described in U.S. Pat. No. 9,051,453, may be a protein that has an amino acid sequence represented by SEQ ID NO: 65, which is also described in U.S. Pat. No. 9,051,453, in which one or a plurality of amino acids have been substituted, deleted, inserted and/or added and that has a repetitious region composed of a crystal region and an amorphous region.

Further, an example of the polypeptide containing two or more units of the amino acid sequence represented by the formula 1: REP1-REP2 (1) is a recombinant protein derived from ADF4 having an amino acid sequence represented by SEQ ID NO: 67, which is also described in U.S. Pat. No. 9,051,453, which is incorporated by reference herein in its entirety. The amino acid sequence represented by SEQ ID NO: 67, which is also described in U.S. Pat. No. 9,051,453, is an amino acid sequence obtained by adding the amino acid sequence (SEQ ID NO: 66, which is also described in U.S. Pat. No. 9,051,453) composed of a start codon, His 10 tags and an HRV3C Protease (Human rhinovirus 3C Protease) recognition site, to the N-terminal of a partial amino acid sequence of ADF4 obtained from the NCBI database (NCBI Accession No.: AAC47011, GI: 1263289). Further, the polypeptide containing two or more units of the amino acid sequence represented by the formula 1: REP1-REP2 (1) may be a polypeptide that has an amino acid sequence represented by SEQ ID NO: 67, which is also described in U.S. Pat. No. 9,051,453, in which one or a plurality of amino acids have been substituted, deleted, inserted and/or added and that has a repetitious region composed of a crystal region and an amorphous region. Further, an example of the polypeptide containing two or more units of the amino acid sequence represented by the formula 1: REP1-REP2 (1) is a recombinant protein derived from MaSp2 that has an amino acid sequence represented by SEQ ID NO: 68, which is also described in of U.S. Pat. No. 9,051,453, which is incorporated by reference here in its entirety. The amino acid sequence represented by SEQ ID NO: 68, which is also described in of U.S. Pat. No. 9,051,453, is an amino acid sequence obtained by adding the amino acid sequence (SEQ ID NO: 66, which is also described in of U.S. Pat. No. 9,051,453,) composed of a start codon, His 10 tags and an HRV3C Protease (Human rhinovirus 3C Protease) recognition site, to the N-terminal of a partial sequence of MaSp2 obtained from the NCBI web database (NCBI Accession No.: AAT75313, GI: 50363147). Furthermore, the polypeptide containing two or more units of the amino acid sequence represented by the formula 1: REP1-REP2 (1) may be a polypeptide that has an amino acid sequence represented by SEQ ID NO: 68, which is also described in of U.S. Pat. No. 9,051,453, in which one or a plurality of amino acids have been substituted, deleted, inserted and/or added and that has a repetitious region composed of a crystal region and an amorphous region.

Examples of the polypeptide derived from flagelliform silk proteins include a polypeptide containing 10 or more units of an amino acid sequence represented by the formula 2: REP3 (2), preferably a polypeptide containing 20 or more units thereof, and more preferably a polypeptide containing 30 or more units thereof. In the case of producing a recombinant protein using a microbe such as Escherichia coli as a host, the molecular weight of the polypeptide derived from flagelliform silk proteins is preferably 500 kDa or less, more preferably 300 kDa or less, and further preferably 200 kDa or less, in terms of productivity.

In the formula (2), the REP 3 indicates an amino acid sequence composed of Gly-Pro-Gly-Gly-X (SEQ ID NO: 69), where X indicates an amino acid selected from the group consisting of Ala, Ser, Tyr and Val.

A major characteristic of the spider silk is that the flagelliform silk does not have a crystal region, but has a repetitious region composed of an amorphous region. Since the major dragline silk and the like have a repetitious region composed of a crystal region and an amorphous region, they are expected to have both high stress and stretchability. Meanwhile, as to the flagelliform silk, although the stress is inferior to that of the major dragline silk, the stretchability is high. The reason for this is considered to be that most of the flagelliform silk is composed of amorphous regions.

An example of the polypeptide containing 10 or more units of the amino acid sequence represented by the formula 2: REP3 (2) is a recombinant protein derived from flagelliform silk proteins having an amino acid sequence represented by SEQ ID NO: 70, which is also described in U.S. Pat. No. 9,051,453, which is incorporated by reference herein in its entirety. The amino acid sequence represented by SEQ ID NO: 70, which is also described in U.S. Pat. No. 9,051,453, is an amino acid sequence obtained by combining a partial sequence of flagelliform silk protein of Nephila clavipes obtained from the NCBI database (NCBI Accession No.: AAF36090, GI: 7106224), specifically, an amino acid sequence thereof from the 1220^{th} residue to the 1659^{th} residue from the N-terminal that corresponds to repetitive sections and motifs (referred to as a PR1 sequence), with a partial sequence of flagelliform silk protein of Nephila clavipes obtained from the NCBI database (NCBI Accession No.: AAC38847, GI: 2833649), specifically, a C-terminal amino acid sequence thereof from the 816^{th} residue to the 907^{th} residue from the C-terminal, and thereafter adding the amino acid sequence (SEQ ID NO: 66, which is also described in U.S. Pat. No. 9,051,453,) composed of a start codon, His 10 tags and an HRV3C Protease recognition site, to the N-terminal of the combined sequence. Further, the polypeptide containing 10 or more units of the amino acid sequence represented by the formula 2: REP3 (2) may be a polypeptide that has an amino acid sequence represented by SEQ ID NO: 70, which is also described in U.S. Pat. No. 9,051,453, in which one or a plurality of amino acids have been substituted, deleted, inserted and/or added and that has a repetitious region composed of an amorphous region.

The polypeptide can be produced using a host that has been transformed by an expression vector containing a gene encoding a polypeptide. A method for producing a gene is not limited particularly, and it may be produced by amplifying a gene encoding a natural spider silk protein from a cell derived from spiders by a polymerase chain reaction (PCR), etc., and cloning it, or may be synthesized chemically. Also, a method for chemically synthesizing a gene is not limited particularly, and it can be synthesized as follows, for example: based on information of amino acid sequences of natural spider silk proteins obtained from the NCBI web database, etc., oligonucleotides that have been synthesized automatically with AKTA oligopilot plus 10/100 (GE Healthcare Japan Corporation) are linked by PCR, etc. At this time, in order to facilitate the purification and observation of protein, it is possible to synthesize a gene that encodes a protein having an amino acid sequence of the above-described amino acid sequence to the N-terminal of which has been added an amino acid sequence composed of a start codon and His 10 tags.

Examples of the expression vector include a plasmid, a phage, a virus, and the like that can express protein based on a DNA sequence. The plasmid-type expression vector is not limited particularly as long as it allows a target gene to be expressed in a host cell and it can amplify itself. For example, in the case of using Escherichia coli Rosetta (DE3) as a host, a pET22b(+) plasmid vector, a pCold plasmid vector, and the like can be used. Among these, in terms of productivity of protein, it is preferable to use the pET22b(+) plasmid vector. Examples of the host include animal cells, plant cells, microbes, etc.

The polypeptide used in the present disclosure is preferably a polypeptide derived from ADF3, which is one of two principal dragline silk proteins of Araneus diadematus. This polypeptide has advantages of basically having high strength-elongation and toughness and of being synthesized easily.

Accordingly, the recombinant silk protein (e.g., the recombinant spider silk-based protein) used in accordance with the embodiments, articles, and/or methods described herein, may include one or more recombinant silk proteins described above or recited in U.S. Pat. Nos. 8,173,772, 8,278,416, 8,618,255, 8,642,734, 8,691,581, 8,729,235, 9,115,204, 9,157,070, 9,309,299, 9,644,012, 9,708,376, 9,051,453, 9,617,315, 9,968,682, 9,689,089, 9,732,125, 9,856,308, 9,926,348, 10,065,997, 10,316,069, and 10,329,332; and U.S. Patent Publication Nos. 2009/0226969, 2011/0281273, 2012/0041177, 2013/0065278, 2013/0115698, 2013/0316376, 2014/0058066, 2014/0079674, 2014/0245923, 2015/0087046, 2015/0119554, 2015/0141618, 2015/0291673, 2015/0291674, 2015/0239587, 2015/0344542, 2015/0361144, 2015/0374833, 2015/0376247, 2016/0024464, 2017/0066804, 2017/0066805, 2015/0293076, 2016/0222174, 2017/0283474, 2017/0088675, 2019/0135880, 2015/0329587, 2019/0040109, 2019/0135881, 2019/0177363, 2019/0225646, 2019/0233481, 2019/0031842, 2018/0355120, 2019/0186050, 2019/0002644, 2020/0031887, 2018/0273590, 20191/094403, 2019/0031843, 2018/0251501, 2017/0066805, 2018/0127553, 2019/0329526, 2020/0031886, 2018/0080147, 2019/0352349, 2020/0043085, 2019/0144819, 2019/0228449, 2019/0340666, 2020/0000091, 2019/0194710, 2019/0151505, 2018/0265555, 2019/0352330, 2019/0248847, and 2019/0378191, the entirety of which are incorporated herein by reference.

### Silk Fibroin-Like Protein Fragments

The recombinant silk protein in this disclosure comprises synthetic proteins which are based on repeat units of natural silk proteins. Besides the synthetic repetitive silk protein sequences, these can additionally comprise one or more natural nonrepetitive silk protein sequences. As used herein, “silk fibroin-like protein fragments” refer to protein fragments having a molecular weight and polydispersity as defined herein, and a certain degree of homology to a protein selected from native silk protein, fibroin heavy chain, fibroin light chain, or any protein comprising one or more GAGAGS (SEQ ID NO: 2) hexa amino acid repeating units. In some embodiments, a degree of homology is selected from about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, or less than 75%.

As described herein, a protein such as native silk protein, fibroin heavy chain, fibroin light chain, or any protein comprising one or more GAGAGS (SEQ ID NO: 2) hexa amino acid repeating units includes between about 9% and about 45% glycine, or about 9% glycine, or about 10% glycine, about 43% glycine, about 44% glycine, about 45% glycine, or about 46% glycine. As described herein, a protein such as native silk protein, fibroin heavy chain, fibroin light chain, or any protein comprising one or more GAGAGS (SEQ ID NO: 2) hexa amino acid repeating units includes between about 13% and about 30% alanine, or about 13% alanine, or about 28% alanine, or about 29% alanine, or about 30% alanine, or about 31% alanine. As described herein, a protein such as native silk protein, fibroin heavy chain, fibroin light chain, or any protein comprising one or more GAGAGS (SEQ ID NO: 2) hexa amino acid repeating units includes between 9% and about 12% serine, or about 9% serine, or about 10% serine, or about 11% serine, or about 12% serine.

In some embodiments, a silk fibroin-like protein described herein includes about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, or about 55% glycine. In some embodiments, a silk fibroin-like protein described herein includes about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, or about 39% alanine. In some embodiments, a silk fibroin-like protein described herein includes about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, or about 22% serine. In some embodiments, a silk fibroin-like protein described herein may include independently any amino acid known to be included in natural fibroin. In some embodiments, a silk fibroin-like protein described herein may exclude independently any amino acid known to be included in natural fibroin. In some embodiments, on average 2 out of 6 amino acids, 3 out of 6 amino acids, or 4 out of 6 amino acids in a silk fibroin-like protein described herein is glycine. In some embodiments, on average 1 out of 6 amino acids, 2 out of 6 amino acids, or 3 out of 6 amino acids in a silk fibroin-like protein described herein is alanine. In some embodiments, on average none out of 6 amino acids, 1 out of 6 amino acids, or 2 out of 6 amino acids in a silk fibroin-like protein described herein is serine.

### Sericin or Sericin Fragments

The main body of the raw silk is silk fibroin fiber, and the silk fibroin fiber is coated with an adhesive substance silk sericin. Sericin is a colloidal silk protein that covers the surface of the silk thread and is composed of bulky amino acids rich in chemical reactivity such as serine, threonine, and aspartic acid, in addition to glycine and alanine. In the various processes of producing silk from raw silk, sericin is important in controlling the solubility of silk and producing high quality silk. Moreover, it plays an extremely important role as an adhesion functional protein. When silk fiber is used as a clothing material, most of the silk sericin covering the silk thread is removed and discarded, so sericin is a valuable unused resource.

In some embodiments, the silk protein fragments described herein include sericin or sericin fragments. Methods of preparing sericin or sericin fragments and their applications in various fields are known and are described herein, and are also described, for example, in U.S. Pat. Nos. 7,115,388, 7,157,273, and 9,187,538, all of which are incorporated by reference herein in their entireties.

In some embodiments, sericin removed from the raw silk cocoons, such as in a degumming step, can be collected and used in the methods described herein. Sericin can also be reconstituted from a powder, and used within the compositions and methods of the disclosure.

### Other Properties of SPF

Compositions of the present disclosure are “biocompatible” or otherwise exhibit “biocompatibility” meaning that the compositions are compatible with living tissue or a living system by not being toxic, injurious, or physiologically reactive and not causing immunological rejection or an inflammatory response. Such biocompatibility can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely. For example, in some embodiments, the coatings described herein are biocompatible coatings.

In some embodiments, compositions described herein, which may be biocompatible compositions (e.g., biocompatible coatings that include silk), may be evaluated and comply with International Standard ISO 10993-1, titled the “Biological evaluation of medical devices—Part 1: Evaluation and testing within a risk management process.” In some embodiments, compositions described herein, which may be biocompatible compositions, may be evaluated under ISO 106993-1 for one or more of cytotoxicity, sensitization, hemocompatibility, pyrogenicity, implantation, genotoxicity, carcinogenicity, reproductive and developmental toxicity, and degradation.

Compositions of the present disclosure are “hypoallergenic” meaning that they are relatively unlikely to cause an allergic reaction. Such hypoallergenicity can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely.

In an embodiment, the stability of a composition of the present disclosure is about 1 day. In an embodiment, the stability of a composition of the present disclosure is about 2 days. In an embodiment, the stability of a composition of the present disclosure is about 3 days. In an embodiment, the stability of a composition of the present disclosure is about 4 days. In an embodiment, the stability of a composition of the present disclosure is about 5 days. In an embodiment, the stability of a composition of the present disclosure is about 6 days. In an embodiment, the stability of a composition of the present disclosure is about 7 days. In an embodiment, the stability of a composition of the present disclosure is about 8 days. In an embodiment, the stability of a composition of the present disclosure is about 9 days. In an embodiment, the stability of a composition of the present disclosure is about 10 days.

In an embodiment, the stability of a composition of the present disclosure is about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days.

In an embodiment, the stability of a composition of the present disclosure is 10 days to 6 months. In an embodiment, the stability of a composition of the present disclosure is 6 months to 12 months. In an embodiment, the stability of a composition of the present disclosure is 12 months to 18 months. In an embodiment, the stability of a composition of the present disclosure is 18 months to 24 months. In an embodiment, the stability of a composition of the present disclosure is 24 months to 30 months. In an embodiment, the stability of a composition of the present disclosure is 30 months to 36 months. In an embodiment, the stability of a composition of the present disclosure is 36 months to 48 months. In an embodiment, the stability of a composition of the present disclosure is 48 months to 60 months.

In an embodiment, a SPF composition of the present disclosure is not soluble in an aqueous solution due to the crystallinity of the protein. In an embodiment, a SPF composition of the present disclosure is soluble in an aqueous solution. In an embodiment, the SPF of a composition of the present disclosure include a crystalline portion of about two-thirds and an amorphous region of about one-third. In an embodiment, the SPF of a composition of the present disclosure include a crystalline portion of about one-half and an amorphous region of about one-half. In an embodiment, the SPF of a composition of the present disclosure include a 99% crystalline portion and a 1% amorphous region. In an embodiment, the SPF of a composition of the present disclosure include a 95% crystalline portion and a 5% amorphous region. In an embodiment, the SPF of a composition of the present disclosure include a 90% crystalline portion and a 10% amorphous region. In an embodiment, the SPF of a composition of the present disclosure include a 85% crystalline portion and a 15% amorphous region. In an embodiment, the SPF of a composition of the present disclosure include a 80% crystalline portion and a 20% amorphous region. In an embodiment, the SPF of a composition of the present disclosure include a 75% crystalline portion and a 25% amorphous region. In an embodiment, the SPF of a composition of the present disclosure include a 70% crystalline portion and a 30% amorphous region. In an embodiment, the SPF of a composition of the present disclosure include a 65% crystalline portion and a 35% amorphous region. In an embodiment, the SPF of a composition of the present disclosure include a 60% crystalline portion and a 40% amorphous region. In an embodiment, the SPF of a composition of the present disclosure include a 50% crystalline portion and a 50% amorphous region. In an embodiment, the SPF of a composition of the present disclosure include a 40% crystalline portion and a 60% amorphous region. In an embodiment, the SPF of a composition of the present disclosure include a 35% crystalline portion and a 65% amorphous region. In an embodiment, the SPF of a composition of the present disclosure include a 30% crystalline portion and a 70% amorphous region. In an embodiment, the SPF of a composition of the present disclosure include a 25% crystalline portion and a 75% amorphous region. In an embodiment, the SPF of a composition of the present disclosure include a 20% crystalline portion and a 80% amorphous region. In an embodiment, the SPF of a composition of the present disclosure include a 15% crystalline portion and a 85% amorphous region. In an embodiment, the SPF of a composition of the present disclosure include a 10% crystalline portion and a 90% amorphous region. In an embodiment, the SPF of a composition of the present disclosure include a 5% crystalline portion and a 90% amorphous region. In an embodiment, the SPF of a composition of the present disclosure include a 1% crystalline portion and a 99% amorphous region.

As used herein, the term “substantially free of inorganic residuals” means that the composition exhibits residuals of 0.1% (w/w) or less. In an embodiment, substantially free of inorganic residuals refers to a composition that exhibits residuals of 0.05% (w/w) or less. In an embodiment, substantially free of inorganic residuals refers to a composition that exhibits residuals of 0.01% (w/w) or less. In an embodiment, the amount of inorganic residuals is between 0 ppm (“non-detectable” or “ND”) and 1000 ppm. In an embodiment, the amount of inorganic residuals is ND to about 500 ppm. In an embodiment, the amount of inorganic residuals is ND to about 400 ppm. In an embodiment, the amount of inorganic residuals is ND to about 300 ppm. In an embodiment, the amount of inorganic residuals is ND to about 200 ppm. In an embodiment, the amount of inorganic residuals is ND to about 100 ppm. In an embodiment, the amount of inorganic residuals is between 10 ppm and 1000 ppm.

As used herein, the term “substantially free of organic residuals” means that the composition exhibits residuals of 0.1% (w/w) or less, in an embodiment, substantially free of organic residuals refers to a composition that exhibits residuals of 0.05% (w/w) or less. In an embodiment, substantially free of organic residuals refers to a composition that exhibits residuals of 0.01% (w/w) or less. In an embodiment, the amount of organic residuals is between 0 ppm (“non-detectable” or “ND”) and 1000 ppm. In an embodiment, the amount of organic residuals is ND to about 500 ppm. In an embodiment, the amount of organic residuals is ND to about 400 ppm. In an embodiment, the amount of organic residuals is ND to about 300 ppm. In an embodiment, the amount of organic residuals is ND to about 200 ppm. In an embodiment, the amount of organic residuals is ND to about 100 ppm. In an embodiment, the amount of organic residuals is between 10 ppm and 1000 ppm.

Compositions of the present disclosure exhibit “biocompatibility” meaning that the compositions are compatible with living tissue or a living system by not being toxic, injurious, or physiologically reactive and not causing immunological rejection. Such biocompatibility can be evidenced by participants topically applying compositions of the present disclosure on their skin for an extended period of time. In an embodiment, the extended period of time is about 3 days. In an embodiment, the extended period of time is about 7 days, in an embodiment, the extended period of time is about 14 days, in an embodiment, the extended period of time is about 21 days. In an embodiment, the extended period of time is about 30 days. In an embodiment, the extended period of time is selected from the group consisting of about I month, about 2 months, about 3 months, about 4 months, about 5 months, about 6 months, about 7 months, about 8 months, about 9 months, about 10 months, about 11 months, about 12 months, and indefinitely.

Compositions of the present disclosure are “hypoallergenic” meaning that they are relatively unlikely to cause an allergic reaction. Such hypoallergenicity can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely.

Following are non-limiting examples of suitable ranges for various parameters in and for preparation of the silk solutions of the present disclosure. The silk solutions of the present disclosure may include one or more, but not necessarily all, of these parameters and may be prepared using various combinations of ranges of such parameters.

In an embodiment, the percent SPF in the solution is less than 30.0 wt. %. In an embodiment, the percent SPF in the solution is less than 25.0 wt. %. In an embodiment, the percent SPF in the solution is less than 20.0 wt. %. In an embodiment, the percent SPF in the solution is less than 19.0 wt. %. In an embodiment, the percent SPF in the solution is less than 18.0 wt. %. In an embodiment, the percent SPF in the solution is less than 17.0 wt. %. In an embodiment, the percent SPF in the solution is less than 16.0 wt. %. In an embodiment, the percent SPF in the solution is less than 15.0 wt. %. In an embodiment, the percent SPF in the solution is less than 14.0 wt. %. In an embodiment, the percent SPF in the solution is less than 13.0 wt. %. In an embodiment, the percent SPF in the solution is less than 12.0 wt. %. In an embodiment, the percent SPF in the solution is less than 11.0 wt. %. In an embodiment, the percent SPF in the solution is less than 10.0 wt. %. In an embodiment, the percent SPF in the solution is less than 9.0 wt. %. In an embodiment, the percent SPF in the solution is less than 8.0 wt. %. In an embodiment, the percent SPF in the solution is less than 7.0 wt. %. In an embodiment, the percent SPF in the solution is less than 6.0 wt. %. In an embodiment, the percent SPF in the solution is less than 5.0 wt. %. In an embodiment, the percent SPF in the solution is less than 4.0 wt. %. In an embodiment, the percent SPF in the solution is less than 3.0 wt. %. In an embodiment, the percent SPF in the solution is less than 2.0 wt. %. In an embodiment, the percent SPF in the solution is less than 1.0 wt. %. In an embodiment, the percent SPF in the solution is less than 0.9 wt. %. In an embodiment, the percent SPF in the solution is less than 0.8 wt. %. In an embodiment, the percent SPF in the solution is less than 0.7 wt. %. In an embodiment, the percent SPF in the solution is less than 0.6 wt. %. In an embodiment, the percent SPF in the solution is less than 0.5 wt. %. In an embodiment, the percent SPF in the solution is less than 0.4 wt. %. In an embodiment, the percent SPF in the solution is less than 0.3 wt. %. In an embodiment, the percent SPF in the solution is less than 0.2 wt. %. In an embodiment, the percent SPF in the solution is less than 0.1 wt. %.

In an embodiment, the percent SPF in the solution is greater than 0.1 wt. %. In an embodiment, the percent SPF in the solution is greater than 0.2 wt. %. In an embodiment, the percent SPF in the solution is greater than 0.3 wt. %. In an embodiment, the percent SPF in the solution is greater than 0.4 wt. %. In an embodiment, the percent SPF in the solution is greater than 0.5 wt. %. In an embodiment, the percent SPF in the solution is greater than 0.6 wt. %. In an embodiment, the percent SPF in the solution is greater than 0.7 wt. %. In an embodiment, the percent SPF in the solution is greater than 0.8 wt. %. In an embodiment, the percent SPF in the solution is greater than 0.9 wt. %. In an embodiment, the percent SPF in the solution is greater than 1.0 wt. %. In an embodiment, the percent SPF in the solution is greater than 2.0 wt. %. In an embodiment, the percent SPF in the solution is greater than 3.0 wt. %. In an embodiment, the percent SPF in the solution is greater than 4.0 wt. %. In an embodiment, the percent SPF in the solution is greater than 5.0 wt. %. In an embodiment, the percent SPF in the solution is greater than 6.0 wt. %. In an embodiment, the percent SPF in the solution is greater than 7.0 wt. %. In an embodiment, the percent SPF in the solution is greater than 8.0 wt. %. In an embodiment, the percent SPF in the solution is greater than 9.0 wt. %. In an embodiment, the percent SPF in the solution is greater than 10.0 wt. %. In an embodiment, the percent SPF in the solution is greater than 11.0 wt. %. In an embodiment, the percent SPF in the solution is greater than 12.0 wt. %. In an embodiment, the percent SPF in the solution is greater than 13.0 wt. %. In an embodiment, the percent SPF in the solution is greater than 14.0 wt. %. In an embodiment, the percent SPF in the solution is greater than 15.0 wt. %. In an embodiment, the percent SPF in the solution is greater than 16.0 wt. %. In an embodiment, the percent SPF in the solution is greater than 17.0 wt. %. In an embodiment, the percent SPF in the solution is greater than 18.0 wt. %. In an embodiment, the percent SPF in the solution is greater than 19.0 wt. %. In an embodiment, the percent SPF in the solution is greater than 20.0 wt. %. In an embodiment, the percent SPF in the solution is greater than 25.0 wt. %.

In an embodiment, the percent SPF in the solution ranges from about 0.1 wt. % to about 30.0 wt. %. In an embodiment, the percent SPF in the solution ranges from about 0.1 wt. % to about 25.0 wt. %. In an embodiment, the percent SPF in the solution ranges from about 0.1 wt. % to about 20.0 wt. %. In an embodiment, the percent SPF in the solution ranges from about 0.1 wt. to about 15.0 wt. %. In an embodiment, the percent SPF in the solution ranges from about 0.1 wt. % to about 10.0 wt. %. In an embodiment, the percent SPF in the solution ranges from about 0.1 wt. % to about 9.0 wt. %. In an embodiment, the percent SPF in the solution ranges from about 0.1 wt. % to about 8.0 wt. %. In an embodiment, the percent SPF in the solution ranges from about 0.1 wt. % to about 7.0 wt. %. In an embodiment, the percent SPF in the solution ranges from about 0.1 wt. % to about 6.5 wt. %. In an embodiment, the percent SPF in the solution ranges from about 0.1 wt. % to about 6.0 wt. %. In an embodiment, the percent SPF in the solution ranges from about 0.1 wt. % to about 5.5 wt. %. In an embodiment, the percent SPF in the solution ranges from about 0.1 wt. % to about 5.0 wt. %. In an embodiment, the percent SPF in the solution ranges from about 0.1 wt. % to about 4.5 wt. %. In an embodiment, the percent SPF in the solution ranges from about 0.1 wt. % to about 4.0 wt. %. In an embodiment, the percent SPF in the solution ranges from about 0.1 wt. % to about 3.5 wt. %. In an embodiment, the percent SPF in the solution ranges from about 0.1 wt. % to about 3.0 wt. %. In an embodiment, the percent SPF in the solution ranges from about 0.1 wt. % to about 2.5 wt. %. In an embodiment, the percent SPF in the solution ranges from about 0.1 wt. % to about 2.0 wt. %. In an embodiment, the percent SPF in the solution ranges from about 0.1 wt. % to about 2.4 wt. %. In an embodiment, the percent SPF in the solution ranges from about 0.5 wt. % to about 5.0 wt. %. In an embodiment, the percent SPF in the solution ranges from about 0.5 wt. % to about 4.5 wt. %. In an embodiment, the percent SPF in the solution ranges from about 0.5 wt. % to about 4.0 wt. %. In an embodiment, the percent SPF in the solution ranges from about 0.5 wt. % to about 3.5 wt. %. In an embodiment, the percent SPF in the solution ranges from about 0.5 wt. % to about 3.0 wt. %. In an embodiment, the percent SPF in the solution ranges from about 0.5 wt. % to about 2.5 wt. %. In an embodiment, the percent SPF in the solution ranges from about 1.0 wt. % to about 4.0 wt. %. In an embodiment, the percent SPF in the solution ranges from about 1.0 wt. % to about 3.5 wt. %. In an embodiment, the percent SPF in the solution ranges from about 1.0 wt. % to about 3.0 wt. %. In an embodiment, the percent SPF in the solution ranges from about 1.0 wt. % to about 2.5 wt. %. In an embodiment, the percent SPF in the solution ranges from about 1.0 wt. % to about 2.4 wt. %. In an embodiment, the percent SPF in the solution ranges from about 1.0 wt. % to about 2.0 wt. %.

In an embodiment, the percent SPF in the solution ranges from about 20.0 wt. % to about 30.0 wt. %. In an embodiment, the percent SPF in the solution ranges from about 0.1 wt. % to about 10.0 wt. %. In an embodiment, the percent SPF in the solution ranges from about 1.0 wt. % to about 10.0 wt. %. In an embodiment, the percent SPF in the solution ranges from about 2 wt. % to about 10.0 wt. %. In an embodiment, the percent SPF in the solution ranges from about 0.1 wt. % to about 6.0 wt. %. In an embodiment, the percent SPF in the solution ranges from about 6.0 wt. % to about 10.0 wt. %. In an embodiment, the percent SPF in the solution ranges from about 6.0 wt. % to about 8.0 wt. %. In an embodiment, the percent SPF in the solution ranges from about 6.0 wt. % to about 9.0 wt. %. In an embodiment, the percent SPF in the solution ranges from about 10.0 wt. % to about 20.0 wt. %. In an embodiment, the percent SPF in the solution ranges from about 11.0 wt. % to about 19.0 wt. %. In an embodiment, the percent SPF in the solution ranges from about 12.0 wt. % to about 18.0 wt. %. In an embodiment, the percent SPF in the solution ranges from about 13.0 wt. % to about 17.0 wt. %. In an embodiment, the percent SPF in the solution ranges from about 14.0 wt. % to about 16.0 wt. %. In an embodiment, the percent SPF in the solution is about 1.0 wt. %. In an embodiment, the percent SPF in the solution is about 1.5 wt. %. In an embodiment, the percent SPF in the solution is about 2.0 wt. %. In an embodiment, the percent SPF in the solution is about 2.4 wt. %. In an embodiment, the percent SPF in the solution is 3.0 wt. %. In an embodiment, the percent SPF in the solution is 3.5 wt. %. In an embodiment, the percent SPF in the solution is about 4.0 wt. %. In an embodiment, the percent SPF in the solution is about 4.5 wt. %. In an embodiment, the percent SPF in the solution is about 5.0 wt. %. In an embodiment, the percent SPF in the solution is about 5.5 wt. %. In an embodiment the percent SPF in the solution is about 6.0 wt. %. In an embodiment, the percent SPF in the solution is about 6.5 wt. %. In an embodiment, the percent SPF in the solution is about 7.0 wt. %. In an embodiment, the percent SPF in the solution is about 7.5 wt. %. In an embodiment, the percent SPF in the solution is about 8.0 wt. %. In an embodiment, the percent SPF in the solution is about 8.5 wt. %. In an embodiment, the percent SPF in the solution is about 9.0 wt. %. In an embodiment, the percent SPF in the solution is about 9.5 wt. %. In an embodiment, the percent SPF in the solution is about 10.0 wt. %.

In an embodiment, the percent sericin in the solution is non-detectable to 25.0 wt. %. In an embodiment, the percent sericin in the solution is non-detectable to 5.0 wt. %. In an embodiment, the percent sericin in the solution is 1.0 wt. %. In an embodiment, the percent sericin in the solution is 2.0 wt. %. In an embodiment, the percent sericin in the solution is 3.0 wt. %. In an embodiment, the percent sericin in the solution is 4.0 wt. %. In an embodiment, the percent sericin in the solution is 5.0 wt. %. In an embodiment, the percent sericin in the solution is 10.0 wt. %. In an embodiment, the percent sericin in the solution is 25.0 wt. %.

In some embodiments, the silk fibroin protein fragments of the present disclosure are shelf stable (they will not slowly or spontaneously gel when stored in an aqueous solution and there is no aggregation of fragments and therefore no increase in molecular weight over time), from 10 days to 3 years depending on storage conditions, percent SPF, and number of shipments and shipment conditions. Additionally, pH may be altered to extend shelf life and/or support shipping conditions by preventing premature folding and aggregation of the silk. In an embodiment, the stability of the LiBr-silk fragment solution is 0 to 1 year. In an embodiment, the stability of the LiBr-silk fragment solution is 0 to 2 years. In an embodiment, the stability of the LiBr-silk fragment solution is 0 to 3 years. In an embodiment, the stability of the LiBr-silk fragment solution is 0 to 4 years. In an embodiment, the stability of the LiBr-silk fragment solution is 0 to 5 years. In an embodiment, the stability of the LiBr-silk fragment solution is 1 to 2 years. In an embodiment, the stability of the LiBr-silk fragment solution is 1 to 3 years. In an embodiment, the stability of the LiBr-silk fragment solution is 1 to 4 years. In an embodiment, the stability of the LiBr-silk fragment solution is 1 to 5 years. In an embodiment, the stability of the LiBr-silk fragment solution is 2 to 3 years. In an embodiment, the stability of the LiBr-silk fragment solution is 2 to 4 years. In an embodiment, the stability of the LiBr-silk fragment solution is 2 to 5 years. In an embodiment, the stability of the LiBr-silk fragment solution is 3 to 4 years. In an embodiment, the stability of the LiBr-silk fragment solution is 3 to 5 years. In an embodiment, the stability of the LiBr-silk fragment solution is 4 to 5 years.

In an embodiment, the stability of a composition of the present disclosure is 10 days to 6 months. In an embodiment, the stability of a composition of the present disclosure is 6 months to 12 months. In an embodiment, the stability of a composition of the present disclosure is 12 months to 18 months. In an embodiment, the stability of a composition of the present disclosure is 18 months to 24 months. In an embodiment, the stability of a composition of the present disclosure is 24 months to 30 months. In an embodiment, the stability of a composition of the present disclosure is 30 months to 36 months. In an embodiment, the stability of a composition of the present disclosure is 36 months to 48 months. In an embodiment, the stability of a composition of the present disclosure is 48 months to 60 months.

In an embodiment, a composition of the present disclosure having SPF has non-detectable levels of LiBr residuals. In an embodiment, the amount of the LiBr residuals in a composition of the present disclosure is between 10 ppm and 1000 ppm. In an embodiment, the amount of the LiBr residuals in a composition of the present disclosure is between 10 ppm and 300 ppm. In an embodiment, the amount of the LiBr residuals in a composition of the present disclosure is less than 25 ppm. In an embodiment, the amount of the Li Br residuals in a composition of the present disclosure is less than 50 ppm. In an embodiment, the amount of the LiBr residuals in a composition of the present disclosure is less than 75 ppm. In an embodiment, the amount of the LiBr residuals in a composition of the present disclosure is less than 100 ppm. In an embodiment, the amount of the LiBr residuals in a composition of the present disclosure is less than 200 ppm. In an embodiment, the amount of the LiBr residuals in a composition of the present disclosure is less than 300 ppm. In an embodiment, the amount of the LiBr residuals in a composition of the present disclosure is less than 400 ppm. In an embodiment, the amount of the LiBr residuals in a composition of the present disclosure is less than 500 ppm. In an embodiment, the amount of the LiBr residuals in a composition of the present disclosure is less than 600 ppm. In an embodiment, the amount of the LiBr residuals in a composition of the present disclosure is less than 700 ppm. In an embodiment, the amount of the LiBr residuals in a composition of the present disclosure is less than 800 ppm. In an embodiment, the amount of the LiBr residuals in a composition of the present disclosure is less than 900 ppm. In an embodiment, the amount of the LiBr residuals in a composition of the present disclosure is less than 1000 ppm. In an embodiment, the amount of the LiBr residuals in a composition of the present disclosure is non-detectable to 500 ppm. In an embodiment, the amount of the LiBr residuals in a composition of the present disclosure is non-detectable to 450 ppm. In an embodiment, the amount of the LiBr residue in a composition of the present disclosure is non-detectable to 400 ppm. In an embodiment, the amount of the LiBr residuals in a composition of the present disclosure is non-detectable to 350 ppm. In an embodiment, the amount of the LiBr residuals in a composition of the present disclosure is non-detectable to 300 ppm. In an embodiment, the amount of the LiBr residuals in a composition of the present disclosure is non-detectable to 250 ppm. In an embodiment, the amount of the LiBr residuals in a composition of the present disclosure is non-detectable to 200 ppm. In an embodiment, the amount of the LiBr residuals in a composition of the present disclosure is non-detectable to 150 ppm. In an embodiment, the amount of the LiBr residuals in a composition of the present disclosure is non-detectable to 100 ppm. In an embodiment, the amount of the LiBr residuals in a composition of the present disclosure is 100 ppm to 200 ppm. In an embodiment, the amount of the LiBr residuals in a composition of the present disclosure is 200 ppm to 300 ppm. In an embodiment, the amount of the LiBr residuals in a composition of the present disclosure is 300 ppm to 400 ppm. In an embodiment, the amount of the LiBr residuals in a composition of the present disclosure is 400 ppm to 500 ppm.

In an embodiment, a composition of the present disclosure having SPF, has non-detectable levels of Na₂CO₃ residuals. In an embodiment, the amount of the Na₂CO₃ residuals in a composition of the present disclosure is less than 100 ppm. In an embodiment, the amount of the Na₂CO₃ residuals in a composition of the present disclosure is less than 200 ppm. In an embodiment, the amount of the Na₂CO₃ residuals in a composition of the present disclosure is less than 300 ppm. In an embodiment, the amount of the Na₂CO₃ residuals in a composition of the present disclosure is less than 400 ppm. In an embodiment, the amount of the Na₂CO₃ residuals in a composition of the present disclosure is less than 500 ppm. In an embodiment, the amount of the Na₂CO₃ residuals in a composition of the present disclosure is less than 600 ppm. In an embodiment, the amount of the Na₂CO₃ residuals in a composition of the present disclosure is less than 700 ppm. In an embodiment, the amount of the Na₂CO₃ residuals in a composition of the present disclosure is less than 800 ppm. In an embodiment, the amount of the Na₂CO₃ residuals in a composition of the present disclosure is less than 900 ppm. In an embodiment, the amount of the Na₂CO₃ residuals in a composition of the present disclosure is less than 1000 ppm. In an embodiment, the amount of the Na₂CO₃ residuals in a composition of the present disclosure is non-detectable to 500 ppm. In an embodiment, the amount of the Na₂CO₃ residuals in a composition of the present disclosure is non-detectable to 450 ppm. In an embodiment, the amount of the Na₂CO₃ residuals in a composition of the present disclosure is non-detectable to 400 ppm. In an embodiment, the amount of the Na₂CO₃ residuals in a composition of the present disclosure is non-detectable to 350 ppm. In an embodiment, the amount of the Na₂CO₃ residuals in a composition of the present disclosure is non-detectable to 300 ppm. In an embodiment, the amount of the Na₂CO₃ residuals in a composition of the present disclosure is non-detectable to 250 ppm. In an embodiment, the amount of the Na₂CO₃ residuals in a composition of the present disclosure is non-detectable to 200 ppm. In an embodiment, the amount of the Na₂CO₃ residuals in a composition of the present disclosure is non-detectable to 150 ppm. In an embodiment, the amount of the Na₂CO₃ residuals in a composition of the present disclosure is non-detectable to 100 ppm. In an embodiment, the amount of the Na₂CO₃ residuals in a composition of the present disclosure is 100 ppm to 200 ppm. In an embodiment, the amount of the Na₂CO₃ residuals in a composition of the present disclosure is 200 ppm to 300 ppm. In an embodiment, the amount of the Na₂CO₃ residuals in a composition of the present disclosure is 300 ppm to 400 ppm. In an embodiment, the amount of the Na₂CO₃ residuals in a composition of the present disclosure is 400 ppm to 500 ppm.

A unique feature of the SPF compositions of the present disclosure are shelf stability (they will not slowly or spontaneously gel when stored in an aqueous solution and there is no aggregation of fragments and therefore no increase in molecular weight over time), from 10 days to 3 years depending on storage conditions, percent silk, and number of shipments and shipment conditions. Additionally pH may be altered to extend shelf-life and/or support shipping conditions by preventing premature folding and aggregation of the silk. In an embodiment, a SPF solution composition of the present disclosure has a shelf stability for up to 2 weeks at room temperature (RT). In an embodiment, a SPF solution composition of the present disclosure has a shelf stability for up to 4 weeks at RT. In an embodiment, a SPF solution composition of the present disclosure has a shelf stability for up to 6 weeks at RT. In an embodiment, a SPF solution composition of the present disclosure has a shelf stability for up to 8 weeks at RT. In an embodiment, a SPF solution composition of the present disclosure has a shelf stability for up to 10 weeks at RT. In an embodiment, a SPF solution composition of the present disclosure has a shelf stability for up to 12 weeks at RT. In an embodiment, a SPF solution composition of the present disclosure has a shelf stability ranging from about 4 weeks to about 52 weeks at RT.

Table R below shows shelf stability test results for embodiments of SPF compositions of the present disclosure.

TABLE R
Shelf Stability of SPF Compositions of the Present Disclosure

| % Silk | Temperature | Time to Gelation | |
|---|---|---|---|
| 2 | RT | 4 | weeks |
| 2 | 4° C. | >9 | weeks |
| 4 | RT | 4 | weeks |
| 4 | 4° C. | >9 | weeks |
| 6 | RT | 2 | weeks |
| 6 | 4° C. | >9 | weeks |

In some embodiments, the water solubility of the silk film derived from silk fibroin protein fragments as described herein can be modified by solvent annealing (water annealing or methanol annealing), chemical crosslinking, enzyme crosslinking and heat treatment.

In some embodiments, the process of annealing may involve inducing beta-sheet formation in the silk fibroin protein fragment solutions used as a coating material. Techniques of annealing (e.g., increase crystallinity) or otherwise promoting “molecular packing” of silk fibroin-protein based fragments have been described. In some embodiments, the amorphous silk film is annealed to introduce beta-sheet in the presence of a solvent selected from the group of water or organic solvent. In some embodiments, the amorphous silk film is annealed to introduce beta-sheet in the presence of water (water annealing process). In some embodiments, the amorphous silk fibroin protein fragment film is annealed to introduce beta-sheet in the presence of methanol. In some embodiments, annealing (e.g., the beta sheet formation) is induced by addition of an organic solvent. Suitable organic solvents include, but are not limited to methanol, ethanol, acetone, isopropanol, or combination thereof.

In some embodiments, annealing is carried out by so-called “water-annealing” or “water vapor annealing” in which water vapor is used as an intermediate plasticizing agent or catalyst to promote the packing of beta-sheets. In some embodiments, the process of water annealing may be performed under vacuum. Suitable such methods have been described in Jin H-J et al. (2005), Water-stable Silk Films with Reduced Beta-Sheet Content, Advanced Functional Materials, 15: 1241-1247; Xiao H. et al. (2011), Regulation of Silk Material Structure by Temperature-Controlled Water Vapor Annealing, Biomacromolecules, 12(5): 1686-1696.

The important feature of the water annealing process is to drive the formation of crystalline beta-sheet in the silk fibroin protein fragment peptide chain to allow the silk fibroin self-assembling into a continuous film. In some embodiments, the crystallinity of the silk fibroin protein fragment film is controlled by controlling the temperature of water vapor and duration of the annealing. In some embodiments, the annealing is performed at a temperature ranging from about 65° C. to about 110° C. In some embodiments, the temperature of the water is maintained at about 80° C. In some embodiments, annealing is performed at a temperature selected from the group of about 65° C., about 70° C., about 75° C., about 80° C., about 85° C., about 90° C., about 95° C., about 100° C., about 105° C., and about 110° C.

In some embodiments, the annealing process lasts a period of time selected from the group of about 1 minute to about 40 minutes, about 1 minute to about 50 minutes, about 1 minute to about 60 minutes, about 1 minute to about 70 minutes, about 1 minute to about 80 minutes, about 1 minute to about 90 minutes, about 1 minute to about 100 minutes, about 1 minute to about 110 minutes, about 1 minute to about 120 minutes, about 1 minute to about 130 minutes, about 5 minutes to about 40 minutes, about 5 minutes to about 50 minutes, about 5 minutes to about 60 minutes, about 5 minutes to about 70 minutes, about 5 minutes to about 80 minutes, about 5 minutes to about 90 minutes, about 5 minutes to about 100 minutes, about 5 minutes to about 110 minutes, about 5 minutes to about 120 minutes, about 5 minutes to about 130 minutes, about 10 minutes to about 40 minutes, about 10 minutes to about 50 minutes, about 10 minutes to about 60 minutes, about 10 minutes to about 70 minutes, about 10 minutes to about 80 minutes, about 10 minutes to about 90 minutes, about 10 minutes to about 100 minutes, about 10 minutes to about 110 minutes, about 10 minutes to about 120 minutes, about 10 minutes to about 130 minutes, about 15 minutes to about 40 minutes, about 15 minutes to about 50 minutes, about 15 minutes to about 60 minutes, about 15 minutes to about 70 minutes, about 15 minutes to about 80 minutes, about 15 minutes to about 90 minutes, about 15 minutes to about 100 minutes, about 15 minutes to about 110 minutes, about 15 minutes to about 120 minutes, about 15 minutes to about 130 minutes, about 20 minutes to about 40 minutes, about 20 minutes to about 50 minutes, about 20 minutes to about 60 minutes, about 20 minutes to about 70 minutes, about 20 minutes to about 80 minutes, about 20 minutes to about 90 minutes, about 20 minutes to about 100 minutes, about 20 minutes to about 110 minutes, about 20 minutes to about 120 minutes, about 20 minutes to about 130 minutes, about 25 minutes to about 40 minutes, about 25 minutes to about 50 minutes, about 25 minutes to about 60 minutes, about 25 minutes to about 70 minutes, about 25 minutes to about 80 minutes, about 25 minutes to about 90 minutes, about 25 minutes to about 100 minutes, about 25 minutes to about 110 minutes, about 25 minutes to about 120 minutes, about 25 minutes to about 130 minutes, about 30 minutes to about 40 minutes, about 30 minutes to about 50 minutes, about 30 minutes to about 60 minutes, about 30 minutes to about 70 minutes, about 30 minutes to about 80 minutes, about 30 minutes to about 90 minutes, about 30 minutes to about 100 minutes, about 30 minutes to about 110 minutes, about 30 minutes to about 120 minutes, about 30 minutes to about 130 minutes, about 35 minutes to about 40 minutes, about 35 minutes to about 50 minutes, about 35 minutes to about 60 minutes, about 35 minutes to about 70 minutes, about 35 minutes to about 80 minutes, about 35 minutes to about 90 minutes, about 35 minutes to about 100 minutes, about 35 minutes to about 110 minutes, about 35 minutes to about 120 minutes, about 35 minutes to about 130 minutes, about 40 minutes to about 50 minutes, about 40 minutes to about 60 minutes, about 40 minutes to about 70 minutes, about 40 minutes to about 80 minutes, about 40 minutes to about 90 minutes, about 40 minutes to about 100 minutes, about 40 minutes to about 110 minutes, about 40 minutes to about 120 minutes, about 40 minutes to about 130 minutes, about 45 minutes to about 50 minutes, about 45 minutes to about 60 minutes, about 45 minutes to about 70 minutes, about 45 minutes to about 80 minutes, about 45 minutes to about 90 minutes, about 45 minutes to about 100 minutes, about 45 minutes to about 110 minutes, about 45 minutes to about 120 minutes, and about 45 minutes to about 130 minutes. In some embodiments, the annealing process lasts a period of time ranging from about 1 minute to about 60 minutes. In some embodiments, the annealing process lasts a period of time ranging from about 45 minutes to about 60 minutes. The longer water annealing post-processing corresponded an increased crystallinity of silk fibroin protein fragments.

In some embodiments, the annealed silk fibroin protein fragment film is immersing the wet silk fibroin protein fragment film in 100% methanol for 60 minutes at room temperature. The methanol annealing changed the composition of silk fibroin protein fragment film from predominantly amorphous random coil to crystalline antiparallel beta-sheet structure.

In some embodiments, the SPF as described herein can be used to prepare SPF microparticles by precipitation with methanol. Alternative flash drying, fluid-bed drying, spray drying or vacuum drying can be applied to remove water from the silk solution. The SPF powder can then be stored and handled without refrigeration or other special handling procedures. In some embodiments, the SPF powders comprise low molecular weight silk fibroin protein fragments. In some embodiments, the SPF powders comprise mid-molecular weight silk fibroin protein fragments. In some embodiments, the SPF powders comprise a mixture of low molecular weight silk fibroin protein fragments and mid-molecular weight silk fibroin protein fragment.

### 1. Silk Fibroin Protein Fragment Solution

Raw silk from Bombyx mori is composed of two primary proteins: silk fibroin (approximately 75%) and sericin (approximately 25%). Silk fibroin is a fibrous protein with a semi-crystalline structure that provides stiffness and strength. As used herein, the term “silk fibroin” means the fibers of the cocoon of Bombyx mori having a weight average molecular weight of about 370,000 Da.

Conversion of these insoluble silk fibroin fibrils into water-soluble silk fibroin protein fragments requires the addition of a concentrated neutral salt (e.g., 8-10 M lithium bromide), which interferes with inter- and intramolecular ionic and hydrogen bonding that would otherwise render the fibroin protein insoluble in water. Methods of making silk fibroin fragments, and/or compositions thereof, are known and are described for example in U.S. Pat. Nos. 9,187,538, 9,511,012, 9,517,191, 9,522,107, 9,522,108, 9,545,369, and 10,166,177.

In an embodiment, silk protein fragment (SPF) mixture solutions are obtained by dissolving raw unscoured, partially scoured, or scoured silkworm fibers with a neutral lithium bromide salt. The raw silkworm silks are processed under selected temperature and other conditions in order to remove any sericin and achieve the desired weight average molecular weight (Mw) and polydispersity (PD) of the fragment mixture. Select process parameters may be altered to achieve distinct final silk protein fragment characteristics depending upon the intended use. The resulting final fragment solution is silk fibroin protein fragments and water with parts per million (ppm) to non-detectable levels of process contaminants, levels acceptable in the pharmaceutical, medical and consumer cosmetic markets. The concentration, size and polydispersity of silk fibroin protein fragments in the solution may further be altered depending upon the desired use and performance requirements.

In an embodiment, silk protein fragment solutions useful for applications in personal care products are prepared according to the following steps: forming pieces of silk cocoons from the Bombyx mori silkworm; extracting the pieces at about 100° C. in a Na₂CO₃ water solution for about 60 minutes, wherein a volume of the water equals about 0.4× raw silk weight and the amount of Na₂CO₃ is about 0.848× the weight of the pieces to form a silk fibroin extract; triple rinsing the silk fibroin extract at about 60° C. for about 20 minutes per rinse in a volume of rinse water, wherein the rinse water for each cycle equals about 0.2 L×the weight of the pieces; removing excess water from the silk fibroin extract; drying the silk fibroin extract; dissolving the dry silk fibroin extract in a LiBr solution, wherein the LiBr solution is first heated to about 100° C. to create a silk and LiBr solution and maintained; placing the silk and LiBr solution in a dry oven at about 100° C. for about 60 minutes to achieve complete dissolution and further fragmentation of the native silk protein structure into mixture with desired molecular weight and polydispersity; filtering the solution to remove any remaining debris from the silkworm; diluting the solution with water to result in a 1.0 wt. % silk solution; and removing solvent from the solution using Tangential Flow Filtration (TFF). In an embodiment, a 10 kDa membrane is utilized to purify the silk solution and create the final desired silk-to-water ratio. TFF can then be used to further concentrate the silk solution to a concentration of 2.0 wt. % silk in water.

Without wishing to be bound by any particular theory, varying extraction (i.e., time and temperature), LiBr (i.e., temperature of LiBr solution when added to silk fibroin extract or vice versa) and dissolution (i.e., time and temperature) parameters results in solvent and silk solutions with different viscosities, homogeneities, and colors. Also without wishing to be bound by any particular theory, increasing the temperature for extraction, lengthening the extraction time, using a higher temperature LiBr solution at emersion and over time when dissolving the silk and increasing the time at temperature (e.g., in an oven as shown here, or an alternative heat source) all resulted in less viscous and more homogeneous solvent and silk solutions.

In an embodiment, solutions of silk fibroin-based protein fragments having a weight average ranging from about 6 kDa to about 17 kDa are prepared according to following steps: degumming a silk source by adding the silk source to a boiling (100° C.) aqueous solution of sodium carbonate for a treatment time of between about 30 minutes to about 60 minutes; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 60° C. to about 140° C.; maintaining the solution of silk fibroin-lithium bromide in an oven having a temperature of about 140° C. for a period of at least 1 hour; removing the lithium bromide from the silk fibroin extract; and producing an aqueous solution of silk protein fragments, the aqueous solution comprising: fragments having a weight average molecular weight ranging from about 6 kDa to about 17 kDa, and wherein the aqueous solution of silk fibroin-based protein fragments comprises a polydispersity of between about 1.5 and about 3.0. The method may further comprise drying the silk fibroin extract prior to the dissolving step. The aqueous solution of silk fibroin-based protein fragments may comprise lithium bromide residuals of less than 300 ppm as measured using a high-performance liquid chromatography lithium bromide assay. The aqueous solution of silk fibroin-based protein fragments may comprise sodium carbonate residuals of less than 100 ppm as measured using a high-performance liquid chromatography sodium carbonate assay. The aqueous solution of silk fibroin-based protein fragments may be lyophilized. In some embodiments, the silk fibroin protein fragment solution may be further processed into various forms including gel, powder, and nanofiber.

In an embodiment, solutions of silk fibroin-based protein fragments having a weight average molecular weight ranging from about 17 kDa to about 39 kDa are prepared according to the following steps: adding a silk source to a boiling (100° C.) aqueous solution of sodium carbonate for a treatment time of between about 30 minutes to about 60 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at least 1 hour; removing the lithium bromide from the silk fibroin extract; and producing an aqueous solution of silk fibroin-based protein fragments, wherein the aqueous solution of silk fibroin-based protein fragments comprises lithium bromide residuals of between about 10 ppm and about 300 ppm, wherein the aqueous solution of silk protein fragments comprises sodium carbonate residuals of between about 10 ppm and about 100 ppm, wherein the aqueous solution of silk fibroin-based protein fragments comprises fragments having a weight average molecular weight ranging from about 17 kDa to about 39 kDa, and wherein the aqueous solution of silk fibroin-based protein fragments comprises a polydispersity of between about 1.5 and about 3.0. The method may further comprise drying the silk fibroin extract prior to the dissolving step. The aqueous solution of silk fibroin-based protein fragments may comprise lithium bromide residuals of less than 300 ppm as measured using a high-performance liquid chromatography lithium bromide assay. The aqueous solution of silk fibroin-based protein fragments may comprise sodium carbonate residuals of less than 100 ppm as measured using a high-performance liquid chromatography sodium carbonate assay.

In an embodiment, solutions of silk fibroin-based protein fragments having a weight average molecular weight ranging from about 39 kDa to about 80 kDa are prepared according to the following steps: adding a silk source to a boiling (100° C.) aqueous solution of sodium carbonate for a treatment time of about 30 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at least 1 hour; removing the lithium bromide from the silk fibroin extract; and producing an aqueous solution of silk fibroin-based protein fragments, wherein the aqueous solution of silk fibroin-based protein fragments comprises lithium bromide residuals of between about 10 ppm and about 300 ppm, sodium carbonate residuals of between about 10 ppm and about 100 ppm, fragments having a weight average molecular weight ranging from about 39 kDa to about 80 kDa, and wherein the aqueous solution of silk fibroin-based protein fragments comprises a polydispersity of between about 1.5 and about 3.0. The method may further comprise drying the silk fibroin extract prior to the dissolving step. The aqueous solution of silk fibroin-based protein fragments may comprise lithium bromide residuals of less than 300 ppm as measured using a high-performance liquid chromatography lithium bromide assay. The aqueous solution of silk fibroin-based protein fragments may comprise sodium carbonate residuals of less than 100 ppm as measured using a high-performance liquid chromatography sodium carbonate assay.

In an embodiment, the silk fibroin-based protein fragments in the solution are substantially devoid of sericin, have a weight average molecular weight ranging from about 6 kDa to about 17 kDa, and have a polydispersity ranging from about 1.5 and about 3.0. In an embodiment, the silk fibroin-based protein fragments in the solution are substantially devoid of sericin, have a weight average molecular weight ranging from about 17 kDa to about 39 kDa, and have a polydispersity ranging from about 1.5 and about 3.0. In an embodiment, the silk fibroin-based protein fragments in the solution are substantially devoid of sericin, have a weight average molecular weight ranging from about 39 kDa to about 80 kDa, and have a polydispersity ranging from about 1.5 and about 3.0.

As used herein, the terms “substantially sericin free” or “substantially devoid of sericin” refer to silk fibers in which a majority of the sericin protein has been removed. In an embodiment, silk fibroin that is substantially devoid of sericin refers to silk fibroin having from about 0.01 wt. % to about 10.0 wt. % sericin. In an embodiment, silk fibroin that is substantially devoid of sericin refers to silk fibroin having about 0.01 wt. % to about 9.0 wt. % sericin. In an embodiment, silk fibroin that is substantially devoid of sericin refers to silk fibroin having from about 0.01 wt. % to about 8.0 wt. % sericin. In an embodiment, silk fibroin that is substantially devoid of sericin refers to silk fibroin having from about 0.01 wt. % to about 7.0 wt. % sericin. In an embodiment, silk fibroin that is substantially devoid of sericin refers to silk fibroin having from about 0.01 wt. % to about 6.0 wt. % sericin. In an embodiment, silk fibroin that is substantially devoid of sericin refers to silk fibroin having from about 0.01 wt. % to about 5.0 wt. % sericin. In an embodiment, silk fibroin that is substantially devoid of sericin refers to silk fibroin having from about 0 wt. % to about 4.0 wt. % sericin. In an embodiment, silk fibroin that is substantially devoid of sericin refers to silk fibroin having from about 0.05 wt. % to about 4.0 wt. % sericin. In an embodiment, silk fibroin that is substantially devoid of sericin refers to silk fibroin having from about 0.1 wt. % to about 4.0 wt. % sericin. In an embodiment, silk fibroin that is substantially devoid of sericin refers to silk fibroin having from about 0.5 wt. % to about 4.0 wt. % sericin. In an embodiment, silk fibroin that is substantially devoid of sericin refers to silk fibroin having from about 1.0 wt. % to about 4.0 wt. % sericin. In an embodiment, silk fibroin that is substantially devoid of sericin refers to silk fibroin having from about 1.5 wt. % to about 4.0 wt. % sericin. In an embodiment, silk fibroin that is substantially devoid of sericin refers to silk fibroin having from about 2.0 wt. % to about 4.0 wt. % sericin. In an embodiment, silk fibroin that is substantially devoid of sericin refers to silk fibroin having from about 2.5 wt. % to about 4.0 wt. % sericin. In an embodiment, silk fibroin that is substantially devoid of sericin refers to silk fibroin having a sericin content from about 0.01 wt. % to about 0.1 wt. %. In an embodiment, silk fibroin that is substantially devoid of sericin refers to silk fibroin having a sericin content below about 0.1 wt. %. In an embodiment, silk fibroin that is substantially devoid of sericin refers to silk fibroin having a sericin content below about 0.05 wt. %. In an embodiment, when a silk source is added to a boiling (100° C.) aqueous solution of sodium carbonate for a treatment time of between about 30 minutes to about 60 minutes, a degumming loss of about 26.0 wt. % to about 31.0 wt. % is obtained.

Following are non-limiting examples of suitable ranges for various parameters in and for preparation of the silk solutions of the present disclosure. The silk solutions of the present disclosure may include one or more, but not necessarily all, of these parameters and may be prepared using various combinations of ranges of such parameters.

In an embodiment, the percent silk in the solution is less than 30.0 wt. %. In an embodiment, the percent silk in the solution is less than 25.0 wt. %. In an embodiment, the percent silk in the solution is less than 20.0 wt. %. In an embodiment, the percent silk in the solution is less than 19.0 wt. %. In an embodiment, the percent silk in the solution is less than 18.0 wt. %. In an embodiment, the percent silk in the solution is less than 17.0 wt. %. In an embodiment, the percent silk in the solution is less than 16.0 wt. %. In an embodiment, the percent silk in the solution is less than 15.0 wt. %. In an embodiment, the percent silk in the solution is less than 14.0 wt. %. In an embodiment, the percent silk in the solution is less than 13.0 wt. %. In an embodiment, the percent silk in the solution is less than 12.0 wt. %. In an embodiment, the percent silk in the solution is less than 11.0 wt. %. In an embodiment, the percent silk in the solution is less than 10.0 wt. %. In an embodiment, the percent silk in the solution is less than 9.0 wt. %. In an embodiment, the percent silk in the solution is less than 8.0 wt. %. In an embodiment, the percent silk in the solution is less than 7.0 wt. %. In an embodiment, the percent silk in the solution is less than 6.0 wt. %. In an embodiment, the percent silk in the solution is less than 5.0 wt. %. In an embodiment, the percent silk in the solution is less than 4.0 wt. %. In an embodiment, the percent silk in the solution is less than 3.0 wt. %. In an embodiment, the percent silk in the solution is less than 2.0 wt. %. In an embodiment, the percent silk in the solution is less than 1.0 wt. %. In an embodiment, the percent silk in the solution is less than 0.9 wt. %. In an embodiment, the percent silk in the solution is less than 0.8 wt. %. In an embodiment, the percent silk in the solution is less than 0.7 wt. %. In an embodiment, the percent silk in the solution is less than 0.6 wt. %. In an embodiment, the percent silk in the solution is less than 0.5 wt. %. In an embodiment, the percent silk in the solution is less than 0.4 wt. %. In an embodiment, the percent silk in the solution is less than 0.3 wt. %. In an embodiment, the percent silk in the solution is less than 0.2 wt. %. In an embodiment, the percent silk in the solution is less than 0.1 wt. %.

In an embodiment, the percent silk in the solution is greater than 0.1 wt. %. In an embodiment, the percent silk in the solution is greater than 0.2 wt. %. In an embodiment, the percent silk in the solution is greater than 0.3 wt. %. In an embodiment, the percent silk in the solution is greater than 0.4 wt. %. In an embodiment, the percent silk in the solution is greater than 0.5 wt. %. In an embodiment, the percent silk in the solution is greater than 0.6 wt. %. In an embodiment, the percent silk in the solution is greater than 0.7 wt. %. In an embodiment, the percent silk in the solution is greater than 0.8 wt. %. In an embodiment, the percent silk in the solution is greater than 0.9 wt. %. In an embodiment, the percent silk in the solution is greater than 1.0 wt. %. In an embodiment, the percent silk in the solution is greater than 2.0 wt. %. In an embodiment, the percent silk in the solution is greater than 3.0 wt. %. In an embodiment, the percent silk in the solution is greater than 4.0 wt. %. In an embodiment, the percent silk in the solution is greater than 5.0 wt. %. In an embodiment, the percent silk in the solution is greater than 6.0 wt. %. In an embodiment, the percent silk in the solution is greater than 7.0 wt. %. In an embodiment, the percent silk in the solution is greater than 8.0 wt. %. In an embodiment, the percent silk in the solution is greater than 9.0 wt. %. In an embodiment, the percent silk in the solution is greater than 10.0 wt. %. In an embodiment, the percent silk in the solution is greater than 11.0 wt. %. In an embodiment, the percent silk in the solution is greater than 12.0 wt. %. In an embodiment, the percent silk in the solution is greater than 13.0 wt. %. In an embodiment, the percent silk in the solution is greater than 14.0 wt. %. In an embodiment, the percent silk in the solution is greater than 15.0 wt. %. In an embodiment, the percent silk in the solution is greater than 16.0 wt. %. In an embodiment, the percent silk in the solution is greater than 17.0 wt. %. In an embodiment, the percent silk in the solution is greater than 18.0 wt. %. In an embodiment, the percent silk in the solution is greater than 19.0 wt. %. In an embodiment, the percent silk in the solution is greater than 20.0 wt. %. In an embodiment, the percent silk in the solution is greater than 25.0 wt. %.

In an embodiment, the percent silk in the solution ranges from about 0.1 wt. % to about 30.0 wt. %. In an embodiment, the percent silk in the solution ranges from about 0.1 wt. % to about 25.0 wt. %. In an embodiment, the percent silk in the solution ranges from about 0.1 wt. % to about 20.0 wt. %. In an embodiment, the percent silk in the solution ranges from about 0.1 wt. % to about 15.0 wt. %. In an embodiment, the percent silk in the solution ranges from about 0.1 wt. % to about 10.0 wt. %. In an embodiment, the percent silk in the solution ranges from about 0.1 wt. % to about 9.0 wt. %. In an embodiment, the percent silk in the solution ranges from about 0.1 wt. % to about 8.0 wt. %. In an embodiment, the percent silk in the solution ranges from about 0.1 wt. % to about 7.0 wt. %. In an embodiment, the percent silk in the solution ranges from about 0.1 wt. % to about 6.5 wt. %. In an embodiment, the percent silk in the solution ranges from about 0.1 wt. % to about 6.0 wt. %. In an embodiment, the percent silk in the solution ranges from about 0.1 wt. % to about 5.5 wt. %. In an embodiment, the percent silk in the solution ranges from about 0.1 wt. % to about 5.0 wt. %. In an embodiment, the percent silk in the solution ranges from about 0.1 wt. % to about 4.5 wt. %. In an embodiment, the percent silk in the solution ranges from about 0.1 wt. % to about 4.0 wt. %. In an embodiment, the percent silk in the solution ranges from about 0.1 wt. % to about 3.5 wt. %. In an embodiment, the percent silk in the solution ranges from about 0.1 wt. % to about 3.0 wt. %. In an embodiment, the percent silk in the solution ranges from about 0.1 wt. % to about 2.5 wt. %. In an embodiment, the percent silk in the solution ranges from about 0.1 wt. % to about 2.0 wt. %. In an embodiment, the percent silk in the solution ranges from about 0.1 wt. % to about 2.4 wt. %. In an embodiment, the percent silk in the solution ranges from about 0.5 wt. % to about 5.0 wt. %. In an embodiment, the percent silk in the solution ranges from about 0.5 wt. % to about 4.5 wt. %. In an embodiment, the percent silk in the solution ranges from about 0.5 wt. % to about 4.0 wt. %. In an embodiment, the percent silk in the solution ranges from about 0.5 wt. % to about 3.5 wt. %. In an embodiment, the percent silk in the solution ranges from about 0.5 wt. % to about 3.0 wt. %. In an embodiment, the percent silk in the solution ranges from about 0.5 wt. % to about 2.5 wt. %. In an embodiment, the percent silk in the solution ranges from about 1.0 wt. % to about 4.0 wt. %. In an embodiment, the percent silk in the solution ranges from about 1.0 wt. % to about 3.5 wt. %. In an embodiment, the percent silk in the solution ranges from about 1.0 wt. % to about 3.0 wt. %. In an embodiment, the percent silk in the solution ranges from about 1.0 wt. % to about 2.5 wt. %. In an embodiment, the percent silk in the solution ranges from about 1.0 wt. % to about 2.4 wt. %. In an embodiment, the percent silk in the solution ranges from about 1.0 wt. % to about 2.0 wt. %.

In an embodiment, the percent silk in the solution ranges from about 20.0 wt. % to about 30.0 wt. %. In an embodiment, the percent silk in the solution ranges from about 0.1 wt. % to about 10.0 wt. %. In an embodiment, the percent silk in the solution ranges from about 1.0 wt. % to about 10.0 wt. %. In an embodiment, the percent silk in the solution ranges from about 2 wt. % to about 10.0 wt. %. In an embodiment, the percent silk in the solution ranges from about 0.1 wt. % to about 6.0 wt. %. In an embodiment, the percent silk in the solution ranges from about 6.0 wt. % to about 10.0 wt. %. In an embodiment, the percent silk in the solution ranges from about 6.0 wt. % to about 8.0 wt. %. In an embodiment, the percent silk in the solution ranges from about 6.0 wt. % to about 9.0 wt. %. In an embodiment, the percent silk in the solution ranges from about 10.0 wt. % to about 20.0 wt. %. In an embodiment, the percent silk in the solution ranges from about 11.0 wt. % to about 19.0 wt. %. In an embodiment, the percent silk in the solution ranges from about 12.0 wt. % to about 18.0 wt. %. In an embodiment, the percent silk in the solution ranges from about 13.0 wt. % to about 17.0 wt. %. In an embodiment, the percent silk in the solution ranges from about 14.0 wt. % to about 16.0 wt. %. In an embodiment, the percent silk in the solution is about 1.0 wt. %. In an embodiment, the percent silk in the solution is about 1.5 wt. %. In an embodiment, the percent silk in the solution is about 2.0 wt. %. In an embodiment, the percent silk in the solution is about 2.4 wt. %. In an embodiment, the percent silk in the solution is 3.0 wt. %. In an embodiment, the percent silk in the solution is 3.5 wt. %. In an embodiment, the percent silk in the solution is about 4.0 wt. %. In an embodiment, the percent silk in the solution is about 4.5 wt. %. In an embodiment, the percent silk in the solution is about 5.0 wt. %. In an embodiment, the percent silk in the solution is about 5.5 wt. %. In an embodiment the percent silk in the solution is about 6.0 wt. %. In an embodiment, the percent silk in the solution is about 6.5 wt. %. In an embodiment, the percent silk in the solution is about 7.0 wt. %. In an embodiment, the percent silk in the solution is about 7.5 wt. %. In an embodiment, the percent silk in the solution is about 8.0 wt. %. In an embodiment, the percent silk in the solution is about 8.5 wt. %. In an embodiment, the percent silk in the solution is about 9.0 wt. %. In an embodiment, the percent silk in the solution is about 9.5 wt. %. In an embodiment, the percent silk in the solution is about 10.0 wt. %.

In an embodiment, the percent sericin in the solution is non-detectable to 30.0 wt. %. In an embodiment, the percent sericin in the solution is non-detectable to 5.0 wt. %. In an embodiment, the percent sericin in the solution is 1.0 wt. %. In an embodiment, the percent sericin in the solution is 2.0 wt. %. In an embodiment, the percent sericin in the solution is 3.0 wt. %. In an embodiment, the percent sericin in the solution is 4.0 wt. %. In an embodiment, the percent sericin in the solution is 5.0 wt. %. In an embodiment, the percent sericin in the solution is 10.0 wt. %. In an embodiment, the percent sericin in the solution is 30.0 wt. %.

In some embodiments, the silk fibroin protein fragments of the present disclosure are shelf stable (they will not slowly or spontaneously gel when stored in an aqueous solution and there is no aggregation of fragments and therefore no increase in molecular weight over time), from 10 days to 3 years depending on storage conditions, percent silk, and number of shipments and shipment conditions. Additionally, pH may be altered to extend shelf life and/or support shipping conditions by preventing premature folding and aggregation of the silk. In an embodiment, the stability of the LiBr-silk fragment solution is 0 to 1 year. In an embodiment, the stability of the LiBr-silk fragment solution is 0 to 2 years. In an embodiment, the stability of the LiBr-silk fragment solution is 0 to 3 years. In an embodiment, the stability of the LiBr-silk fragment solution is 0 to 4 years. In an embodiment, the stability of the LiBr-silk fragment solution is 0 to 5 years. In an embodiment, the stability of the LiBr-silk fragment solution is 1 to 2 years. In an embodiment, the stability of the LiBr-silk fragment solution is 1 to 3 years. In an embodiment, the stability of the LiBr-silk fragment solution is 1 to 4 years. In an embodiment, the stability of the LiBr-silk fragment solution is 1 to 5 years. In an embodiment, the stability of the LiBr-silk fragment solution is 2 to 3 years. In an embodiment, the stability of the LiBr-silk fragment solution is 2 to 4 years. In an embodiment, the stability of the LiBr-silk fragment solution is 2 to 5 years. In an embodiment, the stability of the LiBr-silk fragment solution is 3 to 4 years. In an embodiment, the stability of the LiBr-silk fragment solution is 3 to 5 years. In an embodiment, the stability of the LiBr-silk fragment solution is 4 to 5 years.

In an embodiment, the stability of a composition of the present disclosure is 10 days to 6 months. In an embodiment, the stability of a composition of the present disclosure is 6 months to 12 months. In an embodiment, the stability of a composition of the present disclosure is 12 months to 18 months. In an embodiment, the stability of a composition of the present disclosure is 18 months to 24 months. In an embodiment, the stability of a composition of the present disclosure is 24 months to 30 months. In an embodiment, the stability of a composition of the present disclosure is 30 months to 36 months. In an embodiment, the stability of a composition of the present disclosure is 36 months to 48 months. In an embodiment, the stability of a composition of the present disclosure is 48 months to 60 months.

In an embodiment, a composition of the present disclosure includes silk fibroin-based protein fragments having a weight average molecular weight ranging from 6 kDa to 17 kDa. In an embodiment, a composition of the present disclosure includes silk fibroin-based protein fragments having a weight average molecular weight ranging from 17 kDa to 39 kDa. In an embodiment, a composition of the present disclosure includes silk fibroin-based protein fragments having a weight average molecular weight ranging from 39 kDa to 80 kDa. In an embodiment, a composition of the present disclosure includes silk fibroin-based protein fragments having a weight average molecular weight ranging from 40 kDa to 65 kDa. In an embodiment, a composition of the present disclosure includes silk fibroin-based protein fragments having a weight average molecular weight ranging from 1 kDa to 5 kDa. In an embodiment, a composition of the present disclosure includes silk fibroin-based protein fragments having a weight average molecular weight ranging from 5 kDa to 10 kDa. In an embodiment, a composition of the present disclosure includes silk fibroin-based protein fragments having a weight average molecular weight ranging from 10 kDa to 15 kDa. In an embodiment, a composition of the present disclosure includes silk fibroin-based protein fragments having a weight average molecular weight ranging from 15 kDa to 20 kDa. In an embodiment, a composition of the present disclosure includes silk fibroin-based protein fragments having a weight average molecular weight ranging from 20 kDa to 25 kDa. In an embodiment, a composition of the present disclosure includes silk fibroin-based protein fragments having a weight average molecular weight ranging from 25 kDa to 30 kDa. In an embodiment, a composition of the present disclosure includes silk fibroin-based protein fragments having a weight average molecular weight ranging from 30 kDa to 35 kDa. In an embodiment, a composition of the present disclosure includes silk fibroin-based protein fragments having a weight average molecular weight ranging from 35 kDa to 40 kDa. In an embodiment, a composition of the present disclosure includes silk fibroin-based protein fragments having a weight average molecular weight ranging from 40 kDa to 45 kDa. In an embodiment, a composition of the present disclosure includes silk fibroin-based protein fragments having a weight average molecular weight ranging from 45 kDa to 50 kDa. In an embodiment, a composition of the present disclosure includes silk fibroin-based protein fragments having a weight average molecular weight ranging from 50 kDa to 55 kDa. In an embodiment, a composition of the present disclosure includes silk fibroin-based protein fragments having a weight average molecular weight ranging from 55 kDa to 60 kDa. In an embodiment, a composition of the present disclosure includes silk fibroin-based protein fragments having a weight average molecular weight ranging from 60 kDa to 65 kDa. In an embodiment, a composition of the present disclosure includes silk fibroin-based protein fragments having a weight average molecular weight ranging from 65 kDa to 70 kDa. In an embodiment, a composition of the present disclosure includes silk fibroin-based protein fragments having a weight average molecular weight ranging from 70 kDa to 75 kDa. In an embodiment, a composition of the present disclosure includes silk fibroin-based protein fragments having a weight average molecular weight ranging from 75 kDa to 80 kDa. In an embodiment, a composition of the present disclosure includes silk fibroin-based protein fragments having a weight average molecular weight ranging from 80 kDa to 85 kDa. In an embodiment, a composition of the present disclosure includes silk fibroin-based protein fragments having a weight average molecular weight ranging from 85 kDa to 90 kDa. In an embodiment, a composition of the present disclosure includes silk fibroin-based protein fragments having a weight average molecular weight ranging from 90 kDa to 95 kDa. In an embodiment, a composition of the present disclosure includes silk fibroin-based protein fragments having a weight average molecular weight ranging from 95 kDa to 100 kDa. In an embodiment, a composition of the present disclosure includes silk fibroin-based protein fragments having a weight average molecular weight ranging from 100 kDa to 105 kDa. In an embodiment, a composition of the present disclosure includes silk fibroin-based protein fragments having a weight average molecular weight ranging from 105 kDa to 110 kDa. In an embodiment, a composition of the present disclosure includes silk fibroin-based protein fragments having a weight average molecular weight ranging from 110 kDa to 115 kDa. In an embodiment, a composition of the present disclosure includes silk fibroin-based protein fragments having a weight average molecular weight ranging from 115 kDa to 120 kDa. In an embodiment, a composition of the present disclosure includes silk fibroin-based protein fragments having a weight average molecular weight ranging from 120 kDa to 125 kDa. In an embodiment, a composition of the present disclosure includes silk fibroin-based protein fragments having a weight average molecular weight ranging from 125 kDa to 130 kDa. In an embodiment, a composition of the present disclosure includes silk fibroin-based protein fragments having a weight average molecular weight ranging from 130 kDa to 135 kDa. In an embodiment, a composition of the present disclosure includes silk fibroin-based protein fragments having a weight average molecular weight ranging from 135 kDa to 140 kDa. In an embodiment, a composition of the present disclosure includes silk fibroin-based protein fragments having a weight average molecular weight ranging from 140 kDa to 145 kDa. In an embodiment, a composition of the present disclosure includes silk fibroin-based protein fragments having a weight average molecular weight ranging from 145 kDa to 150 kDa. In an embodiment, a composition of the present disclosure includes silk fibroin-based protein fragments having a weight average molecular weight ranging from 150 kDa to 155 kDa. In an embodiment, a composition of the present disclosure includes silk fibroin-based protein fragments having a weight average molecular weight ranging from 155 kDa to 160 kDa. In an embodiment, a composition of the present disclosure includes silk fibroin-based protein fragments having a weight average molecular weight ranging from 160 kDa to 165 kDa. In an embodiment, a composition of the present disclosure includes silk fibroin-based protein fragments having a weight average molecular weight ranging from 165 kDa to 170 kDa. In an embodiment, a composition of the present disclosure includes silk fibroin-based protein fragments having a weight average molecular weight ranging from 170 kDa to 175 kDa. In an embodiment, a composition of the present disclosure includes silk fibroin-based protein fragments having a weight average molecular weight ranging from 175 kDa to 180 kDa. In an embodiment, a composition of the present disclosure includes silk fibroin-based protein fragments having a weight average molecular weight ranging from 180 kDa to 185 kDa. In an embodiment, a composition of the present disclosure includes silk fibroin-based protein fragments having a weight average molecular weight ranging from 185 kDa to 190 kDa. In an embodiment, a composition of the present disclosure includes silk fibroin-based protein fragments having a weight average molecular weight ranging from 190 kDa to 195 kDa. In an embodiment, a composition of the present disclosure includes silk fibroin-based protein fragments having a weight average molecular weight ranging from 195 kDa to 200 kDa. In an embodiment, a composition of the present disclosure includes silk fibroin-based protein fragments having a weight average molecular weight ranging from 200 kDa to 205 kDa. In an embodiment, a composition of the present disclosure includes silk fibroin-based protein fragments having a weight average molecular weight ranging from 205 kDa to 210 kDa. In an embodiment, a composition of the present disclosure includes silk fibroin-based protein fragments having a weight average molecular weight ranging from 210 kDa to 215 kDa. In an embodiment, a composition of the present disclosure includes silk fibroin-based protein fragments having a weight average molecular weight ranging from 215 kDa to 220 kDa. In an embodiment, a composition of the present disclosure includes silk fibroin-based protein fragments having a weight average molecular weight ranging from 220 kDa to 225 kDa. In an embodiment, a composition of the present disclosure includes silk fibroin-based protein fragments having a weight average molecular weight ranging from 225 kDa to 230 kDa. In an embodiment, a composition of the present disclosure includes silk fibroin-based protein fragments having a weight average molecular weight ranging from 230 kDa to 235 kDa. In an embodiment, a composition of the present disclosure includes silk fibroin-based protein fragments having a weight average molecular weight ranging from 235 kDa to 240 kDa. In an embodiment, a composition of the present disclosure includes silk fibroin-based protein fragments having a weight average molecular weight ranging from 240 kDa to 245 kDa. In an embodiment, a composition of the present disclosure includes silk fibroin-based protein fragments having a weight average molecular weight ranging from 245 kDa to 250 kDa. In an embodiment, a composition of the present disclosure includes silk fibroin-based protein fragments having a weight average molecular weight ranging from 250 kDa to 255 kDa. In an embodiment, a composition of the present disclosure includes silk fibroin-based protein fragments having a weight average molecular weight ranging from 255 kDa to 260 kDa. In an embodiment, a composition of the present disclosure includes silk fibroin-based protein fragments having a weight average molecular weight ranging from 260 kDa to 265 kDa. In an embodiment, a composition of the present disclosure includes silk fibroin-based protein fragments having a weight average molecular weight ranging from 265 kDa to 270 kDa. In an embodiment, a composition of the present disclosure includes silk fibroin-based protein fragments having a weight average molecular weight ranging from 270 kDa to 275 kDa. In an embodiment, a composition of the present disclosure includes silk fibroin-based protein fragments having a weight average molecular weight ranging from 275 kDa to 280 kDa. In an embodiment, a composition of the present disclosure includes silk fibroin-based protein fragments having a weight average molecular weight ranging from 280 kDa to 285 kDa. In an embodiment, a composition of the present disclosure includes silk fibroin-based protein fragments having a weight average molecular weight ranging from 285 kDa to 290 kDa. In an embodiment, a composition of the present disclosure includes silk fibroin-based protein fragments having a weight average molecular weight ranging from 290 kDa to 295 kDa. In an embodiment, a composition of the present disclosure includes silk fibroin-based protein fragments having a weight average molecular weight ranging from 295 kDa to 300 kDa. In an embodiment, a composition of the present disclosure includes silk fibroin-based protein fragments having a weight average molecular weight ranging from 300 kDa to 305 kDa. In an embodiment, a composition of the present disclosure includes silk fibroin-based protein fragments having a weight average molecular weight ranging from 305 kDa to 310 kDa. In an embodiment, a composition of the present disclosure includes silk fibroin-based protein fragments having a weight average molecular weight ranging from 310 kDa to 315 kDa. In an embodiment, a composition of the present disclosure includes silk fibroin-based protein fragments having a weight average molecular weight ranging from 315 kDa to 320 kDa. In an embodiment, a composition of the present disclosure includes silk fibroin-based protein fragments having a weight average molecular weight ranging from 320 kDa to 325 kDa. In an embodiment, a composition of the present disclosure includes silk fibroin-based protein fragments having a weight average molecular weight ranging from 325 kDa to 330 kDa. In an embodiment, a composition of the present disclosure includes silk fibroin-based protein fragments having a weight average molecular weight ranging from 330 kDa to 335 kDa. In an embodiment, a composition of the present disclosure includes silk fibroin-based protein fragments having a weight average molecular weight ranging from 350 kDa to 340 kDa. In an embodiment, a composition of the present disclosure includes silk fibroin-based protein fragments having a weight average molecular weight ranging from 340 kDa to 345 kDa. In an embodiment, a composition of the present disclosure includes silk fibroin-based protein fragments having a weight average molecular weight ranging from 345 kDa to 350 kDa.

In an embodiment, the silk fibroin-based protein fragments in this disclosure has a polydispersity ranging from about 1.0 to about 5.0. In an embodiment, a composition of the silk fibroin-based protein fragments has a polydispersity ranging from about 1.5 to about 3.0. In an embodiment, a composition of the silk fibroin-based protein fragments has a polydispersity ranging from about 1.0 to about 1.5. In an embodiment, a composition of the silk fibroin-based protein fragments has a polydispersity ranging from about 1.5 to about 2.0. In an embodiment, a composition of the silk fibroin-based protein fragments has a polydispersity ranging from about 2.0 to about 2.5. In an embodiment, a composition of the silk fibroin-based protein fragments, has a polydispersity ranging from about is 2.0 to about 3.0. In an embodiment, a composition of the silk fibroin-based protein fragments has a polydispersity ranging from about is 2.5 to about 3.0. In some embodiments, the silk solution described above can be dried to a SPF powder. This can be accomplished by placing the silk solution in a lyophilizer at an appropriate temperature (e.g., room temperature), at a pressure of less than about 100 millitorr (mtorr) until the water and other volatiles have been evaporated (about 1.0 wt. % to about 10 wt. % moisture content), and a fine SPF powder remains. The solid silk powder resulted from lyophilization is then pulverized to form fine powders of desired particle size.

In some embodiments, the silk solution as described above can be casted on a substrate to form a silk film containing silk fibroin protein fragments after drying. The silk film is then pulverized to form fine powders.

In some embodiments, the silk solution as described above can be dried by subjecting to thin film evaporation process (also known as Rototherm) followed by milling. The silk solution is placed in a thin film evaporator under reduced pressure, gentle heating and water is continuously removed from the aqueous solution to result in a solid of variable particle size. The particle size can be varied by controlling the evaporation process parameters including pressure, temperature, rotational speed of the cylinder, thickness of the liquid film in the evaporator. The dry protein powder resulted from the rototherm evaporation contains less than 10.0 wt. % moisture content.

In some embodiments, the silk solution as described above can be used to prepare SPF microparticles by precipitation with methanol.

Alternative flash drying, fluid-bed drying, spray drying or vacuum drying can be applied to remove water from the silk solution.

The SPF powder can then be stored and handled without refrigeration or other special handling procedures.

In some embodiments, the SPF powders comprise low molecular weight silk fibroin protein fragments. In some embodiments, the SPF powders comprise mid-molecular weight silk fibroin protein fragments. In some embodiments, the SPF powders comprise a mixture of low molecular weight silk fibroin protein fragments and mid-molecular weight silk fibroin protein fragment.

In some embodiments, the SPF powders comprise low molecular weight silk fibroin protein fragments having a weight average molecular weight ranging from about 5 kDa to about 20 kDa. In some embodiments, the SPF powders comprise low molecular weight silk fibroin protein fragments having an average weight average molecular weight selected from between about 14 kDa to about 30 kDa. In some embodiments, the SPF powders comprise low molecular weight silk fibroin protein fragments having a weight average molecular weight selected from the group consisting of from about 5 kDa to 10 kDa, about 10 kDa to about 20 kDa, and about 20 kDa to about 25 kDa. In some embodiments, the SPF powders comprise low molecular weight silk fibroin protein fragments having a weight average molecular weight ranging from about 10 kDa to about 20 kDa.

In some embodiments, the SPF powders comprise mid-molecular weight silk fibroin protein fragments having an average weight average molecular weight selected from between about 25 kDa to about 30 kDa, from between about 30 kDa to about 35 kDa, from between about 35 kDa to about 40 kDa, from between about 17 kDa to about 39 kDa, from between about 45 kDa to about 50 kDa, from between about 50 kDa to about 55 kDa, from between about 55 kDa to about 60 kDa, from between about 60 kDa to about 65 kDa, from between about 40 kDa to about 65 kDa, from 65 kDa to about 70 kDa, from between about 70 kDa to about 75 kDa, from between about 75 kDa to about 80 kDa, from between about 39 kDa to about 80 kDa, from between about 80 kDa to about 85 kDa, from between about 85 kDa to about 90 kDa, from between about 90 kDa to about 95 kDa, from between about 95 kDa to about 100 kDa, from between about 100 kDa to about 105 kDa, from between about 105 kDa to about 110 kDa, from between about 60 kDa to about 100 kDa, and from between about 80 kDa to about 144 kDa. In some embodiments, the SPF powders comprise mid-molecular weight silk fibroin protein fragments having a weight average molecular weight selected from between about 17 kDa to about 39 kDa. In some embodiments, the SPF powders comprise mid-molecular weight silk fibroin protein fragments having a weight average molecular weight selected from between about 40 kDa to about 65 kDa. In some embodiments, the SPF powders comprise mid-molecular weight silk fibroin protein fragments having a weight average molecular weight selected from between about 39 kDa to about 80 kDa. In some embodiments, the SPF powders comprise mid-molecular weight silk fibroin protein fragments having a weight average molecular weight selected from between about 80 kDa to about 144 kDa.

In some embodiments, the SPF powders comprise low molecular weight silk fibroin fragments (low-MW silk) having a weight average molecular weight (Mw) selected from between about 6 kDa and about 17 kDa and a polydispersity between about 1.5 and about 3.0. In some embodiments, the SPF powders comprise low molecular weight silk fibroin fragments (low-MW silk) having a weight average molecular weight (Mw) selected from between 14 kDa and about 30 kDa and a polydispersity between about 1.5 and about 3.0. In some embodiments, the SPF powders comprise mid-molecular weight silk fibroin fragments (Med-MW silk) having a weight average molecular weight selected from between about 17 kDa and about 39 kDa and a polydispersity between about 1.5 and about 3.0. In some embodiments, the SPF powders comprise mid-molecular weight silk fibroin fragments (high-MW silk) having a weight average molecular weight selected from between about 39 kDa to about 80 kDa and a polydispersity between about 1.5 and about 3.0.

In some embodiments, the moisture content in the SPF powder ranges from 0.1 wt. % to 20 wt. % by the total weight of the SPF powder. In some embodiments, the moisture content in the SPF powder ranges from 1.0 wt. % to 10 wt. % by the total weight of the SPF powder. In some embodiments, the moisture content in the SPF powder is less than 1.0 wt. % by the total weight of the SPF powder. In some embodiments, the moisture content in the SPF powder is less than 5.0 wt. % by the total weight of the SPF powder. In some embodiments, the moisture content in the SPF powder is less than 10.0 wt. % by the total weight of the SPF powder. In some embodiments, the moisture content in the SPF powder is selected from the group consisting of less than 1.0 wt. %, less than 1.5 wt. %, less than 2.0 wt. %, less than 2.5 wt. %, less than 3.0 wt. %, less than 3.5 wt. %, less than 4.0 wt. %, less than 4.5 wt. %, less than 5.0 wt. %, less than 5.5 wt. %, less than 6.0 wt. %, less than 6.5 wt. %, less than 7.0 wt. %, less than 7.5 wt. %, less than 8.0 wt. %, less than 8.5 wt. %, less than 9.0 wt. %, less than 9.5 wt. % and less than 10.0 wt. % by the total weight of the SPF powder.

In some embodiments, the SPF powder are solid particles having median particle size ranging from 1.0 μm to 1000 μm. In some embodiments, the SPF powder are microparticles having median particle size ranging from 1.0 μm to 500 μm. In some embodiments, the SPF powder are microparticles having median particle size ranging from 1.0 μm to 300 μm. In some embodiments, the SPF powder are microparticles having median particle size ranging from 1.0 μm to 250 μm. In some embodiments, the SPF powder are microparticles having median particle size ranging from 1.0 μm to 200 μm. In some embodiments, the SPF powder are microparticles having median particle size ranging from 1.0 μm to 100 μm. In some embodiments, the SPF powder are microparticles having median particle size ranging from 1.0 μm to 50.0 μm. In some embodiments, the SPF powder are microparticles having median particle size ranging from 1.0 μm to 25.0 μm. In some embodiments, the SPF powder are microparticles having median particle size ranging from 1.0 μm to 10.0 μm. In some embodiments, the SPF powder are microparticles having median particle size selected from the group consisting of about 1.0 μm, about 2.0 μm, about 3.0 μm, about 4.0 μm, about 5.0 μm, about 6.0 μm, about 7.0 μm, about 8.0 μm, about 9.0 μm, about 10.0 μm, about 11.0 μm, about 12.0 μm, about 13.0 μm, about 14.0 μm, about 15.0 μm, about 16.0 μm, about 17.0 μm, about 18.0 μm, about 19.0 μm, about 20.0 μm, about 21.0 μm, about 22.0 μm, about 23.0 μm, about 24.0 μm, about 25.0 μm, about 26.0 μm, about 27.0 μm, about 28.0 μm, about 29.0 μm, about 30.0 μm, about 31.0 μm, about 32.0 μm, about 33.0 μm, about 34.0 μm, about 35.0 μm, about 36.0 μm, about 37.0 μm, about 38.0 μm, about 39.0 μm, about 40.0 μm, about 41.0 μm, about 42.0 μm, about 43.0 μm, about 44.0 μm, about 45.0 μm, about 46.0 μm, about 47.0 μm, about 48.0 μm, about 49.0 μm, about 50.0 μm, about 51.0 μm, about 52.0 μm, about 53.0 μm, about 54.0 μm, about 55.0 μm, about 56.0 μm, about 57.0 μm, about 58.0 μm, about 59.0 μm, about 60.0 μm, about 61.0 μm, about 62.0 μm, about 63.0 μm, about 64.0 μm, about 65.0 μm, about 66.0 μm, about 67.0 μm, about 68.0 μm, about 69.0 μm, about 70.0 μm, about 71.0 μm, about 72.0 μm, about 73.0 μm, about 74.0 μm, about 75.0 μm, about 76.0 μm, about 77.0 μm, about 78.0 μm, about 79.0 μm, about 80.0 μm, about 81.0 μm, about 82.0 μm, about 83.0 μm, about 84.0 μm, about 85.0 μm, about 86.0 μm, about 87.0 μm, about 88.0 μm, about 89.0 μm, about 90.0 μm, about 91.0 μm, about 92.0 μm, about 93.0 μm, about 94.0 μm, about 95.0 μm, about 96.0 μm, about 97.0 μm, about 98.0 μm, about 99.0 μm, about 100.0 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, about 500 μm, about 510 μm, about 520 μm, about 530 μm, about 540 μm, about 550 μm, about 560 μm, about 570 μm, about 580 μm, about 590 μm, about 600 μm, about 610 μm, about 620 μm, about 630 μm, about 640 μm, about 650 μm, about 660 μm, about 670 μm, about 680 μm, about 690 μm, about 700 μm, about 710 μm, about 720 μm, about 730 μm, about 740 μm, about 750 μm, about 760 μm, about 770 μm, about 780 μm, about 790 μm, about 800 μm, about 810 μm, about 820 μm, about 830 μm, about 840 μm, about 850 μm, about 860 μm, about 870 μm, about 880 μm, about 890 μm, about 900 μm, about 910 μm, about 920 μm, about 930 μm, about 940 μm, about 950 μm, about 960 μm, about 970 μm, about 980 μm, about 990 μm, and about 1000 μm.

In some embodiments, the SPF powder are microparticles having median particle size less than 500 μm. In some embodiments, the SPF powder are microparticles having median particle size less than 325 μm. In some embodiments, the SPF powder are microparticles having median particle size less than 250 μm. In some embodiments, the SPF powder are microparticles having median particle size less than 100 μm. In some embodiments, the SPF powder are microparticles having median particle size less than 50 μm. In some embodiments, the SPF powder are microparticles having median particle size less than 10 μm.

The silk powder described herein may find application in cosmetics, personal care, house care, food and textile industry.

In some embodiments, the silk microparticles described herein may find applications as active agent for personal care product, for example, as micro-exfoliators or micro-exfoliates, as delivery systems for scents/volatile molecule (e.g., perfume encapsulated silk microparticles), as delivery systems for oral care active agents, as mucoadhesive delivery systems for systemic delivery of therapeutic agent, as mucoadhesive delivery systems for local delivery of therapeutic drug to oral cavity.

In some embodiments, the silk microparticles described herein may find applications as delivery systems for therapeutically active agent, e.g., delivery systems for sustained release of drugs.

In some embodiments, the fibroin protein fragment solution can be freeze dried to form lyophilized silk powder. In some embodiments, lyophilized silk powder can be resuspended in water, hexafluoroisopropanol (HFIP), or organic solution following storage to create silk solutions of varying concentrations, including higher concentration solutions than those produced initially.

In some embodiments, the fibroin protein fragment solution can be casted on a substrate to form a silk fibroin film after drying.

In some embodiments, the silk fibroin-based protein fragments are dried using a rototherm evaporator or other methods known in the art for creating a dry protein form containing less than 10.0% water by mass. In an embodiment, the solubility of silk fibroin-based protein fragments of the present disclosure in organic solutions ranges from about 50.0% to about 100%. In an embodiment, the solubility of silk fibroin-based protein fragments of the present disclosure in organic solutions ranges from about 60.0% to about 100%. In an embodiment, the solubility of silk fibroin-based protein fragments of the present disclosure in organic solutions ranges from about 70.0% to about 100%. In an embodiment, the solubility of silk fibroin-based protein fragments of the present disclosure in organic solutions ranges from about 80.0% to about 100%. In an embodiment, the solubility of silk fibroin-based protein fragments of the present disclosure in organic solutions ranges from about 90.0% to about 100%. In some embodiments, the silk fibroin-based fragments of the present disclosure are non-soluble in organic solutions.

In some embodiments, silk fibroin protein fragments useful for applications in personal care products also include an aqueous gel of the silk fibroin protein fragments. The gelation of silk fibroin protein fragment solutions may be induced by sonication, vortex, heating, solvent treatment (e.g. methanol, ethanol), electrogelation, ultrasonication, chemicals (e.g. vitamin C), or the like.

In some embodiments, the silk fibroin protein fragments comprise cationic quaternized amino acid residue (cationic quaternized silk fibroin) with fatty alkyl groups, wherein the silk fibroin protein fragments having an average weight average molecular weight selected from the group consisting of 5 kDa, 10 kDa, 15 kDa, 20 kDa, 25 kDa, 30 KDa, 35 kDa, 40 kDa, 45 kDa, 50 KDa, 55 kDa, 60 kDa, 65 kDa, 70 kDa, 75 kDa, 80 kDa, 85 kDa, 90 kDa, 95 kDa, and 100 kDa, and a polydispersity of about 1.5 to about 3.0. In some embodiments, the fatty alkyl group for quaternization of amine groups of the silk fibroin fragment is selected from the group consisting of cocodimonium hydroxypropyl, hydroxypropyltrimonium, lauryidimonium hydroxypropyl, steardimonium hydroxypropyl, quaternium-79, and combinations thereof.

Silk peptide is an extract from natural silk fibroin hydrolysate. Silk peptide exhibits pearl luster and silky feel when incorporated into personal care products. The structure of silk peptide is similar to human hair and skin tissue. The silk peptides are serine rich polypeptides having 2 to 50 amino acid residues and weight average molecular weights as described herein. Thus, the silk peptides incorporated in the silk personal care products having high affinity to skin after the application.

### 2. Silk Fibroin Protein Fragment Composition

Emulsions are thermodynamically unstable systems consisting of at least two immiscible fluids, one of which is dispersed in form of droplets in the other. Emulsions are characterized as oil-in-water emulsion (O/W), or water-in-oil emulsion (W/O) depending on the identities of the dispersed droplets and continuous phase.

The emulsion tends to break down over time due to a variety of physicochemical mechanisms, such as gravitational separation or creaming, flocculation, coalescence and Ostwald ripening. Emulsions can be stabilized kinetically by adding emulsifiers with their capability of absorbing at the oil/water interface by lowering the interfacial tension and preventing the droplets from aggregation. Emulsifiers play a central role in forming emulsions that are widely used in cosmetic, encapsulation, drug delivery, material and biomedical fields.

Conventional synthetic amphiphilic surfactants are most often used to stabilize emulsions. Two classes of emulsifiers are commonly used including low molecular weight synthetic surfactants and macromolecular emulsifiers (e.g., proteins) which are used singly or in synergistic combinations.

However, the synthetic surfactant exhibits some disadvantages including potential toxicity, skin irritation, inflammation, and inferior stability toward pH, salts, and temperature.

Copolymers, lipids, proteins, polymersomes, and solid particles are developed to complement conventional synthetic surfactants. Proteins are most desirable due to their biocompatibility, biodegradability, and intrinsic amphiphilic properties. Peptides have also been extensively studied as emulsifiers due to their sequence and size control, biocompatibility, versatility, and stabilizing capacity. However, cost and mass production remains the challenges for broader utility for these protein/peptide emulsifiers.

Thus, the development of new emulsifiers with well-defined stability, biocompatibility, and biodegradability is desired for drug, cosmetic and biomedical applications. Further, the demand for replacement of synthetic surfactant with natural surfactant is on the rise due to the growing demand for healthier, environmental friendly personal care products.

Silk fibroin is an amphiphilic polymer with large hydrophobic domains occupying the backbone component of the peptide chain. The hydrophobic regions are interrupted by small hydrophilic spacers, and the N- and C-termini of the peptide chains are highly hydrophilic. The hydrophobic domains of the fibroin protein H-chain contain a repetitive hexapeptide sequence of Gly-Ala-Gly-Ala-Gly-Ser and repeats of Gly-Ala/Ser/Tyr dipeptides, which can form stable anti-parallel-sheet crystallites. The amino acid sequence of the silk fibroin L-chain is non-repetitive. Therefore, the L-chain is more hydrophilic and relatively elastic. The hydrophilic blocks (Tyr, Ser) and the hydrophobic (Gly, Ala) blocks in silk fibroin molecules are arranged alternatively such that allows self-assembling of silk fibroin molecules. Silk fibroin has a hydrophobic tail like section formed by the Gly-Ala repeats followed by a polar amino acid such as serine such that it behaves as the surfactant head group.

The silk fibroin molecule exhibits surface activity and amphiphilic characteristics because of its hydrophobic and hydrophilic regions arrangements in the polypeptide chain. This allows the silk fibroin to self-assemble at interfaces and form stable viscoelastic films at the surface of the air-water interface or oil-water interface. The stable viscoelastic layers that the silk fibroin creates prevent droplets or bubbles from coalescence as well as macroscopic phase separation.

Silk fibroin and other proteins diffuse and absorb at a slower pace than surfactants to the air-water interface. Once absorbed, the protein begins to change conformation, unfold and form a two dimensional (2D) viscoelastic gel with other molecules. In some embodiments, the storage modulus (G′) instantly rises and then increases further at steady state over time. In some embodiments, the loss modulus (G″) has a decrease initially but does not change with time like the G′ does. The silk fibroin at the interface displays the characteristics of a strong interfacial gel. The elastic moduli values at the silk fibroins surface are substantially larger than other proteins like β-casein, lysozyme, and insulin. Regardless of the concentration of silk protein, it exhibits elastic-like behavior across all frequencies at the air-water interface with G′ dominating over G″. The molecular chains of the silk fibroin display gel-like behavior because of β-sheet crystalline structure in the silk fibroin protein.

Studies in this disclosure on surface active property of silk fibroin fragments and emulsion behavior supported that silk fibroin peptide has the propensity to adsorb at the water-air interface (See Examples 1-5). Once silk fibroin is adsorbed at the air-water interface, interfacial gel-like structures are formed. The adsorption process and the structure formed at the air-water interface are important when assessing the suitability for applications dependent on surface activity. In some embodiments, silk protein can be used as a novel surfactant in the cosmetic industry because of its behavior as a biocompatible emulsion stabilizer.

In one embodiment, the disclosure provides a silk fibroin fragment composition comprising SPF as defined herein, including, without limitation, silk fibroin protein and silk fibroin fragments, and a polydispersity ranging from 1 to about 5, from 0 to 500 ppm lithium bromide, from 0 to 500 ppm sodium carbonate; and at least one emulsifiable component. In some embodiments, the silk fibroin fragments have an average weight average molecular weight selected from between about 1 kDa to about 5 kDa, from between about 5 kDa to about 10 kDa, from between about 6 kDa to about 17 kDa, from between about 10 kDa to about 15 kDa, from between about 15 kDa to about 20 kDa, from between about 17 kDa to about 39 kDa, from between about 20 kDa to about 25 kDa, from between about 25 kDa to about 30 kDa, from between about 30 kDa to about 35 kDa, from between about 35 kDa to about 40 kDa, from between about 39 kDa to about 80 kDa, from between about 40 kDa to about 45 kDa, from between about 45 kDa to about 50 kDa, from between about 60 kDa to about 100 kDa, and from between about 80 kDa to about 144 kDa.

In an embodiment, this disclosure provides a silk fibroin fragment composition comprising silk fibroin fragments having an average weight average molecular weight selected from between about 1 kDa to about 5 kDa, from between about 5 kDa to about 10 kDa, from between about 6 kDa to about 17 kDa, from between about 10 kDa to about 15 kDa, from between about 15 kDa to about 20 kDa, from between about 17 kDa to about 39 kDa, from between about 20 kDa to about 25 kDa, from between about 25 kDa to about 30 kDa, from between about 30 kDa to about 35 kDa, from between about 35 kDa to about 40 kDa, from between about 39 kDa to about 80 kDa, from between about 40 kDa to about 45 kDa, from between about 45 kDa to about 50 kDa, from between about 60 kDa to about 100 kDa, and from between about 80 kDa to about 144 kDa, and a polydispersity ranging from 1 to about 5, from 0 to 500 ppm lithium bromide, from 0 to 500 ppm sodium carbonate; and at least one emulsifiable component.

In some embodiments, the silk fibroin fragments have a polydispersity ranging from about 1 to about 1.5. In some embodiments, the silk fibroin fragments have a polydispersity ranging from about 1.5 to about 2.0. In some embodiments, the silk fibroin fragments have a polydispersity ranging from about 1.5 to about 3.0. In some embodiments, the silk fibroin fragments have a polydispersity ranging from about 2.0 to about 2.5. In some embodiments, the silk fibroin fragments have a polydispersity ranging from about 2.5 to about 3.0.

In some embodiments, the silk fibroin fragments are present at an amount ranging from about 0.01 wt. % to about 10.0 wt. % by the total weight of the silk fibroin fragment composition. In some embodiments, the silk fibroin fragments are present at an amount ranging from at about 0.01 wt. % to about 1.0 wt. % by the total weight of the silk fibroin fragment composition. In some embodiments, the silk fibroin fragments are present at an amount ranging from at about 1.0 wt. % to about 2.0 wt. % by the total weight of the silk fibroin composition. In some embodiments, the silk fibroin fragments are present at an amount ranging from about 2.0 wt. % to about 3.0 wt. % by the total weight of the silk fibroin fragment composition. In some embodiments, the silk fibroin fragments are present at an amount ranging from about 3.0 wt. % to about 4.0 wt. % by the total weight of the silk fibroin fragment composition. In some embodiments, the silk fibroin fragments are present at an amount ranging from about 4.0 wt. % to about 5.0 wt. % by the total weight of the silk fibroin fragment composition. In some embodiments, the silk fibroin fragments are present at an amount ranging from about 5.0 wt. % to about 6.0 wt. % by the total weight of the silk fibroin fragment composition.

In some embodiments, the silk fibroin fragment composition further comprising about 0.01% (w/w) to about 10% (w/w) sericin by the total weight of the silk fibroin fragment composition.

In some embodiments, the silk fibroin fragments do not spontaneously or gradually gelate and do not visibly change in color or turbidity when in an aqueous solution for at least 10 days prior to formulation into the silk fibroin fragment composition.

In some embodiments, the silk fibroin protein fragments alone as described herein can act as emulsifiers to create stable emulsions. The emulsifier system comprises an aqueous solution of silk fibroin protein fragments and is substantially free of any secondary surface-active agent as co-emulsifier. The term “substantially free” of any secondary surface active agent refers to the percent weight amount of the secondary surface active agent present in the emulsion is less than 5.0 wt. % by the total weight of the emulsion. In some embodiments, the term “substantially free of” refers to the percent weight amount of the secondary surface active agent is less than an value selected from about 5.0 Wt. %, about 4.0 wt. %, about 3.0 wt. %, about 2.0 wt. %, about 1.0 wt., about 0.5 wt. %, about 0.1 wt. %, about 0.01 wt. %, about 0.001 wt. %, about 0.0001 wt. %, and 0 wt. % by the total weight of the emulsion.

In some embodiments, the emulsifier comprises low molecular weight silk fibroin protein fragments having an average weight average molecular weight ranging from about 5 kDa to about 20 kDa. In some embodiments, the emulsifier comprises low molecular weight silk fibroin protein fragments having an average weight average molecular weight selected from between about 14 kDa to about 30 kDa. In some embodiments, the emulsifier comprises low molecular weight silk fibroin protein fragments having an average weight average molecular weight selected from the group consisting of from about 5 kDa to 10 kDa, about 10 kDa to about 20 kDa, and about 20 kDa to about 25 kDa. In some embodiments, the emulsifier comprises low molecular weight silk fibroin protein fragments having an average weight average molecular weight ranging from about 10 kDa to about 20 kDa.

In some embodiments, the emulsifier comprises medium molecular weight silk fibroin protein fragments having an average weight average molecular weight selected from between about 25 kDa to about 30 kDa, from between about 30 kDa to about 35 kDa, from between about 35 kDa to about 40 kDa, from between about 17 kDa to about 39 kDa, from between about 45 kDa to about 50 kDa, from between about 50 kDa to about 55 kDa, from between about 55 kDa to about 60 kDa, from between about 60 kDa to about 65 kDa, from between about 40 kDa to about 65 kDa, from between 65 kDa to about 70 kDa, from between about 70 kDa to about 75 kDa, from between about 75 kDa to about 80 kDa, from between about 39 kDa to about 80 kDa, from between about 80 kDa to about 85 kDa, from between about 85 kDa to about 90 kDa, from between about 90 kDa to about 95 kDa, from between about 95 kDa to about 100 kDa, from between about 100 kDa to about 105 kDa, from between about 105 kDa to about 110 kDa, from between about 60 kDa to about 100 kDa, and from between about 80 kDa to about 144 kDa. In some embodiments, the emulsifier comprises medium molecular weight silk fibroin protein fragments having an average weight average molecular weight selected from between about 17 kDa to about 39 kDa. In some embodiments, the emulsifier comprises medium molecular weight silk fibroin protein fragments having an average weight average molecular weight selected from between about 40 kDa to about 65 kDa. In some embodiments, the emulsifier comprises medium molecular weight silk fibroin protein fragments having an average weight average molecular weight selected from between about 39 kDa to about 80 kDa. In some embodiments, the emulsifier comprises medium molecular weight silk fibroin protein fragments having an average weight average molecular weight selected from between about 80 kDa to about 144 kDa.

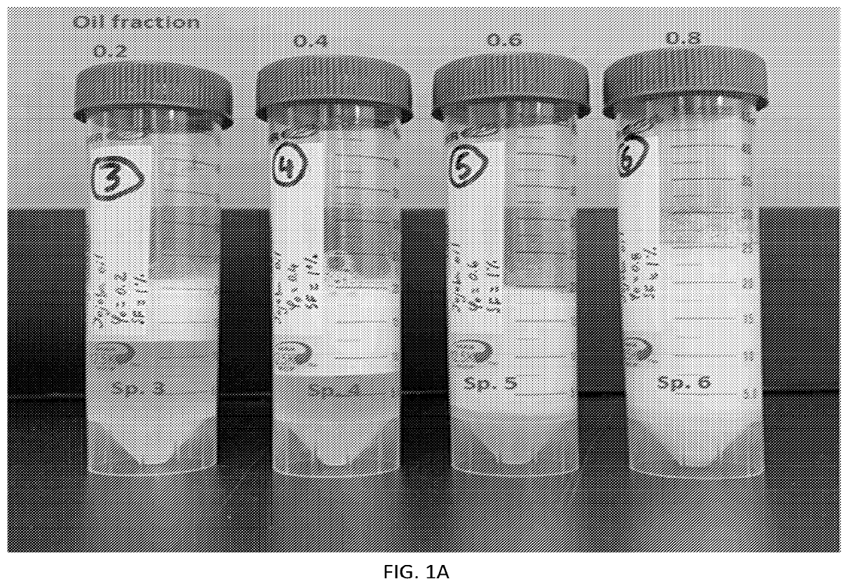

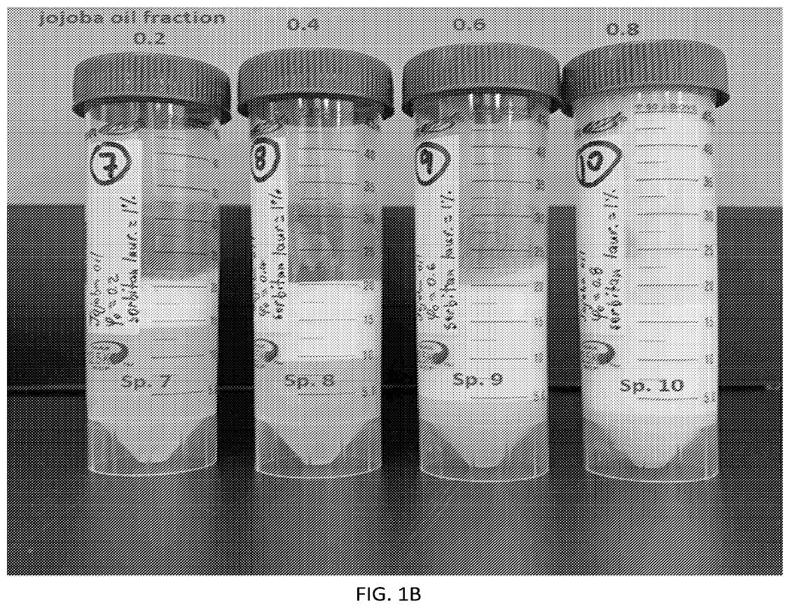

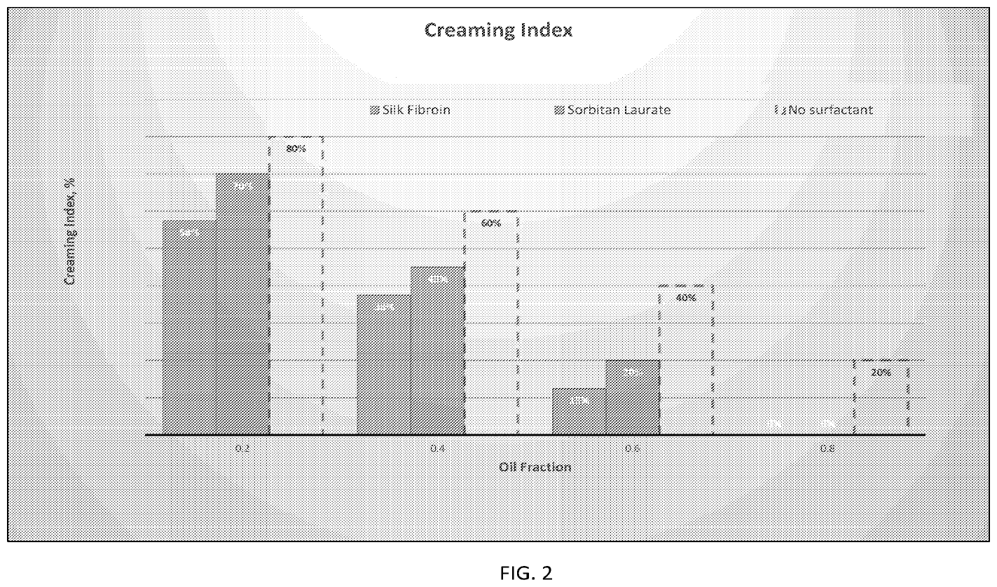

In some embodiments, the silk fibroin protein fragment composition exhibits enhanced emulsification power as compared with whole silk fibroin protein. In some embodiments, the silk fibroin protein fragment solution (calculated HLB=6.2) exhibits better emulsification efficiency as measured by creaming index as compared to the comparable synthetic surfactant sorbitan laurate (Span 20™, HLB=8.6) in an jojoba oil based emulsion with each of the emulsifier used at 1.0% w/v (FIGS. 1A-B and FIG. 2). The creaming index for the silk fibroin protein fragments is of 13.0% to 58.0%, whereas the creaming index for sorbitan laurate is of 20.0% to 70.0%.

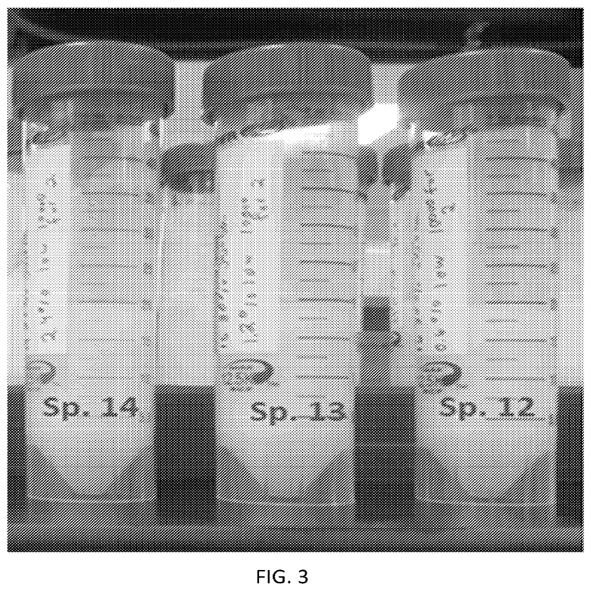

In some embodiments, water in 80 wt. % jojoba oil/squalane emulsion with good creaming stability are produced at silk fibroin concentration selected from the group consisting of about 0.6% w/v, w/w, or v/v; about 1.2% w/v, w/w or v/v; and about 2.4% w/v, w/w or v/v (See FIGS. 3-5).

In some embodiments, the silk fibroin protein emulsifier is present at an amount ranging from about 0.5% w/v, w/w or v/v to about 6.0% w/v, w/w or v/v by the total weight of the emulsion. In some embodiments, the silk fibroin protein emulsifier is present at an amount ranging from about 0.6% w/v, w/w or v/v to about 3.0% w/v, w/w or v/v by the total weight of the emulsion. In some embodiments, the silk fibroin protein emulsifier is present at an amount ranging from about 1.0% w/v, w/w or v/v to about 3.0% w/v, w/w or v/v by the total weight of the emulsion. In some embodiments, the silk fibroin protein emulsifier is present at an amount ranging from about 1.2% w/v, w/w or v/v to about 2.4% w/v, w/w or v/v by the total weight of the emulsion. In some embodiments, the silk fibroin protein emulsifier is present at a weight percent amount selected from the group consisting of about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, and about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, and about 6.0% w/v, w/w or v/v.

In some embodiments, the silk fibroin fragment composition comprises silk fibroin protein nanofibers as emulsifier formed by concentration-dilution process to induce silk assembly in the silk fibroin solution as described above.

In some embodiments, synergistic effects exist for the surfactant blend containing glucoside and dodecylbenzene sulfonate and its surface tension reduction property. Blends of conventional surfactant with biosurfactant for surface tension reduction can also exist. For example, cocamidopropyl betaine (CAPB) was blended with rhamnolipid and/or sophorolipid surfactants as binary and ternary mixtures. It was found that with the addition of the biosurfactants can not only reduce surface tension but also improve surface elasticity. This effect was due to the rhamnolipid dominating the interface and was seen in all binary and ternary mixtures that had the rhamnolipid present in it.

Without wishing to be bound by any particular theory, it is believed that the fibroin protein stability is improved by the surfactants that shield the exposure of the protein's hydrophobic surfaces. This shielding effect prevents the denaturization of the silk fibroin protein at the air-water interface. The surfactants that are used most often to blend with proteins are amphipathic and non-ionic surfactant such as polysorbates. When silk protein is combined with polysorbate 80 in the application to control the release and recovery of antibody, the surfactant functions to disrupt the secondary structure of the silk fibroin protein hydrophobic β-sheet. Others surfactant blend of proteins and surfactants have been explored. It was found that the type of protein and its structure controlled the proteins and surfactants adsorption capacity. Bovine serum albumin showed a higher surface elasticity because of its rigid structure compared to lysozyme. This also made bovine serum albumin more efficient at the interface.

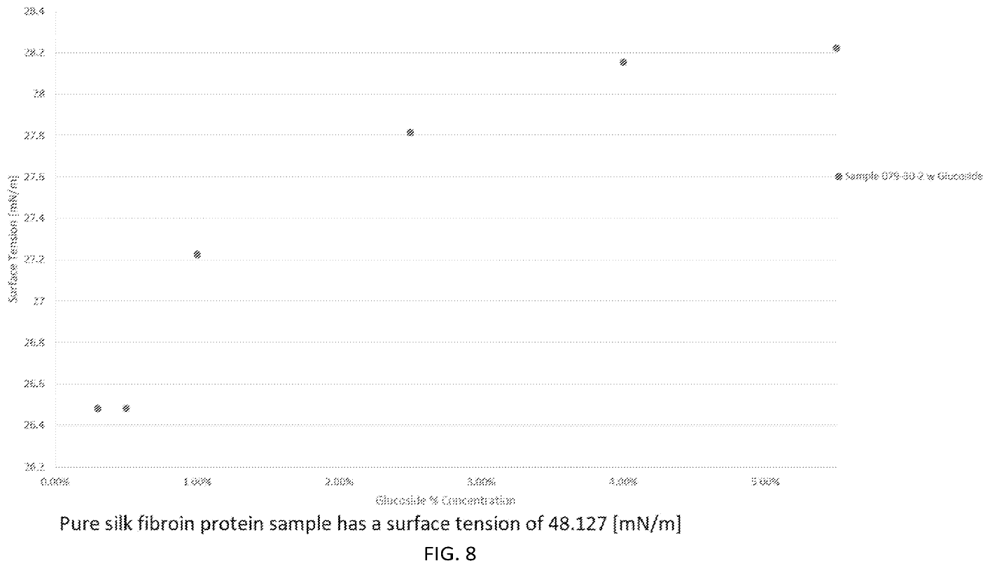

Silk protein alone does not exhibit very high emulsification efficiency (surface tension reduced from 72 mN/m of pure water to 48.127 mN/m for silk-water mixture, a reduction of 24 surface tension units) as compared with that of traditional surfactants (˜35 mN/m, reduction of 37 surface tension units) such as sodium laureth sulfate (SLES), cocamidopropyl betaine (CAPB) and natural surfactant such as glucosides (29 mN/m, reduction of 43 surface tension units), rhamnolipids, or sophorolipids (See FIG. 8).

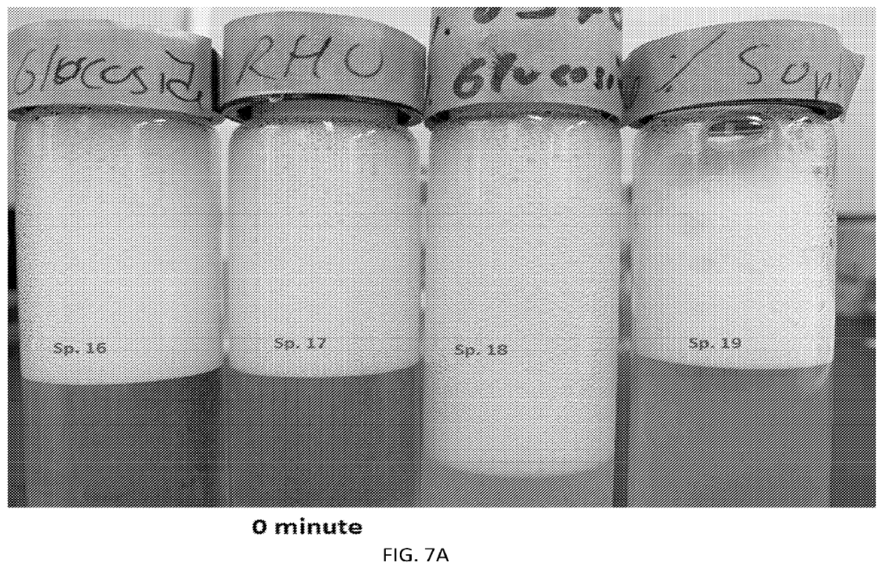

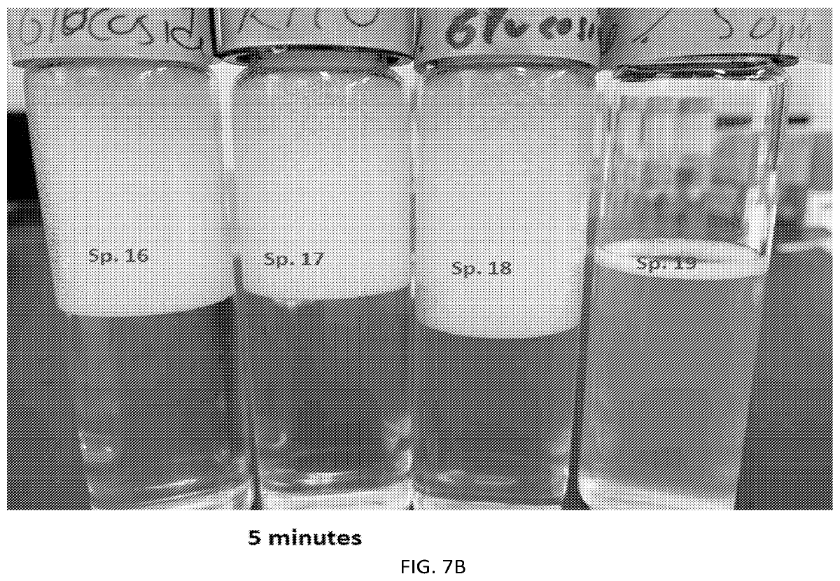

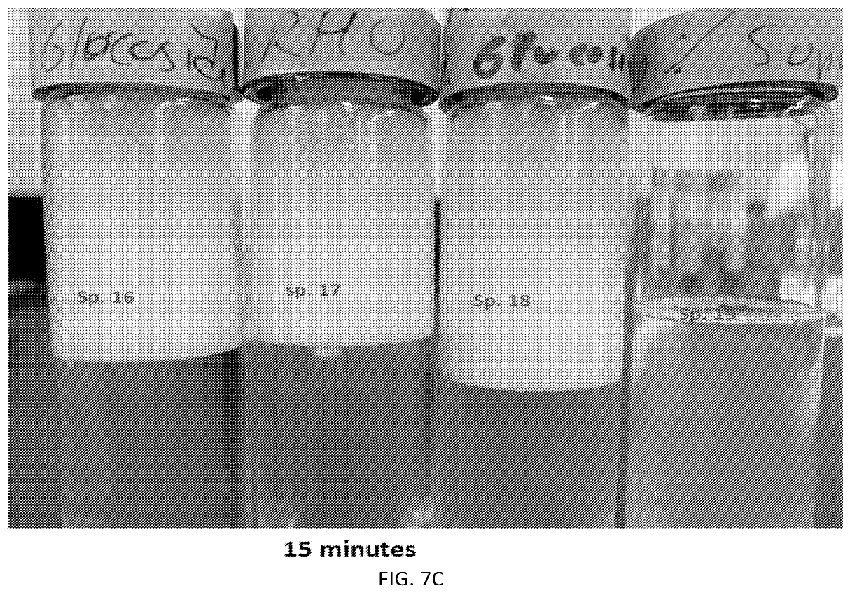

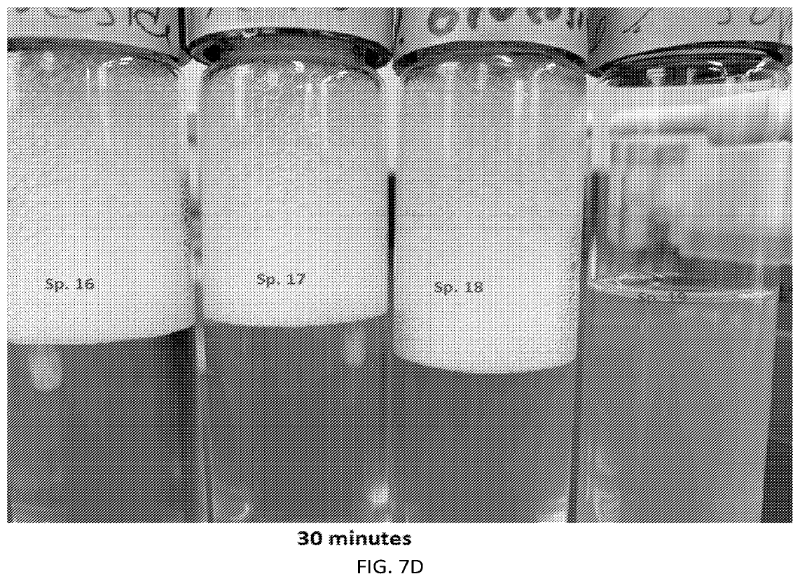

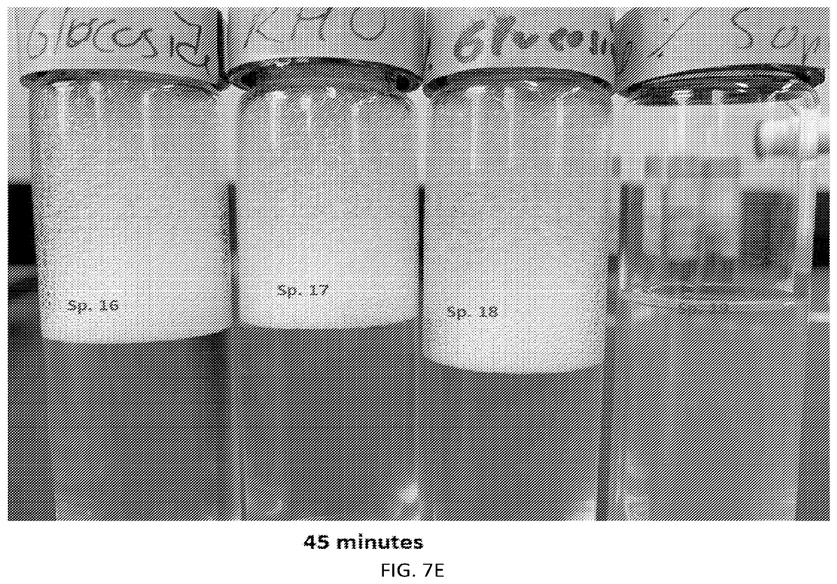

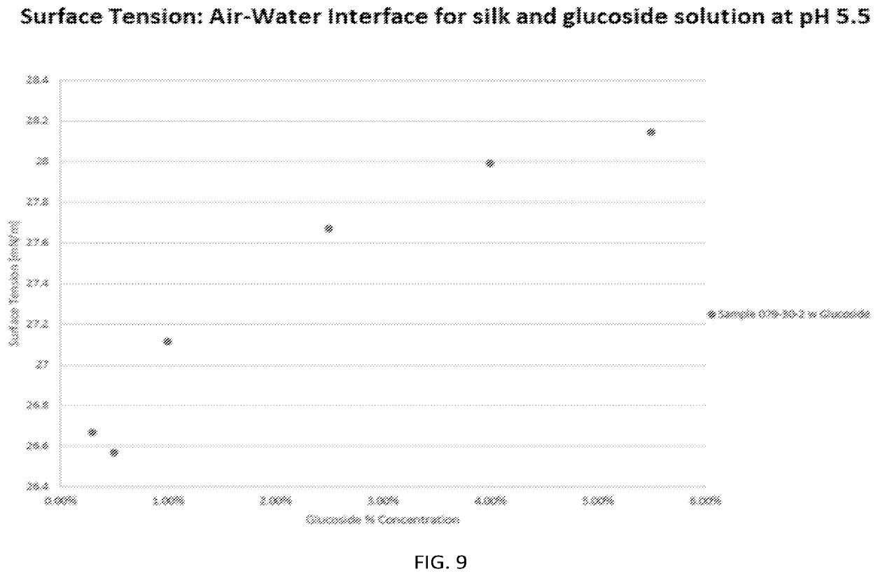

This disclosure discovered surprising synergistic effects on reducing surface tension at the water-gas interface by an emulsifier blend containing a mixture of silk fibroin protein fragments and an alkyl polyglucoside as compared with either of the pure silk fibroin protein or pure glucoside emulsifier. The extremely low surface tension exhibited by the silk fibroin protein/glucoside emulsifier blend resulted in high foam volume generation and good cleansing efficacy (See FIGS. 7A-7E). It was also discovered that lowering pH to 5.5 for the foam stabilized by the silk fibroin protein-glucoside emulsifier blend reduces surface tension slightly (See FIG. 9). In some embodiments, on its own, the silk protein does not seem to be very surface active or efficient at forming strong elastic layers at the air-water interface but in combination with glucoside there is a synergistic effect that results in effectively reducing surface tension. Even when the amount of silk protein increases, surface tension continues to decrease when mixed with glucoside. The combination of silk protein and glucoside reduces surface tension from 44.93 mN/m at 5% pure silk protein to 27.22 mN/m at 5% silk protein and 1% glucoside.

In some embodiments, the emulsifier blend comprises from about 1 wt. % to about 20.0 wt. % of glucoside as co-emulsifier and from about 80 wt. % to about 99 wt. % silk fibroin fragments as primary emulsifier by the total weight of the emulsifier blend. In some embodiments, the emulsifier blend comprises about 16.7 wt. % of glucoside as co-emulsifier and from about 83.3 wt. % silk fibroin fragments as primary emulsifier by the total weight of the emulsifier blend. In some embodiments, the emulsifier blend comprises about 9.0 wt. % of glucoside as co-emulsifier and from about 91.0 wt. % silk fibroin fragments as primary emulsifier by the total weight of the emulsifier blend. In some embodiments, the emulsifier blend comprises about 5.7 wt. % of glucoside as co-emulsifier and from about 94.3 wt. % silk fibroin fragments as primary emulsifier by the total weight of the emulsifier blend. In some embodiments, the weight ratio of glucoside to silk fibroin protein fragments in the blend is of about 1:5. In some embodiments, the weight ratio of glucoside to silk fibroin protein fragments in the blend ranges from about 1:5, 1:6, 1:7, 1:8, 1:9, 1:10, 1:11, 1:12, 1:13, 1:14, 1:15, or 1:16.7. In some embodiments, the weight ratio of the alkyl glucoside emulsifier to the silk fibroin fragments in the blend is a value ranging from about 1:4 to about 1:20. In some embodiments, the weight ratio of the alkyl glucoside emulsifier to the silk fibroin fragments in the blend is a value selected from about 1:5 to about 1:11. In some embodiments, the weight ratio of the alkyl glucoside emulsifier to the silk fibroin fragments in the blend is about 1:5. In some embodiments, the weight ratio of the alkyl glucoside emulsifier to the silk fibroin fragments in the blend is about 1:11.

In some embodiments, the weight ratio of the alkyl glucoside emulsifier to the silk fibroin fragments in the blend is about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99.

In some embodiments, the silk fibroin fragment composition comprises about 0.2% w/w, w/v or v/v to about 1.0% w/w, w/v or v/v of the alkyl glucoside emulsifier and about 1.0% w/w, w/v or v/v to about 5.0% w/w, w/v or v/v of the silk fibroin protein fragment, wherein the alkyl glucoside emulsifier is selected from the group consisting of cetearyl glucoside, caprylyl/capryl glucoside, and combinations thereof. In some embodiments, the silk fibroin fragment composition comprises about 1.0% w/w, w/v or v/v of the alkyl glucoside emulsifier and about 5.0% w/w, w/v or v/v of silk fibroin protein fragment, wherein the alkyl glucoside emulsifier is selected from the group consisting of cetearyl glucoside, caprylyl/capryl glucoside, and combinations thereof. In some embodiments, the alkyl glucoside emulsifier is caprylyl/capryl glucoside.

In some embodiments, the alkyl glucoside emulsifier is present in an amount ranging from about 0.5% w/w, w/v or v/v to about 1.0% w/w, w/v or v/v of and the silk fibroin protein fragment is present in an amount ranging from about 5.0 w/w, w/v or v/v to about 5.5 w/w, w/v or v/v by the basis of the silk fibroin fragment composition. In some embodiments, the alkyl glucoside emulsifier is present in an amount of about 0.5% w/w and the silk fibroin protein fragment is present in an amount of about 5.5 w/w by the basis of the silk fibroin fragment composition. In some embodiments, the alkyl glucoside emulsifier is present in an amount of about 1 w/w and the silk fibroin protein fragment is present in an amount of about 5.0 w/w by the basis of the silk fibroin fragment composition.

In some embodiments, the alkyl glucoside emulsifier is caprylyl/capryl glucoside. In some embodiments, the silk fibroin protein fragment composition comprises from about 0.01% w/w, w/v or v/v to about 0.1% w/w, w/v or v/v of caprylyl/capryl glucoside and about 0.01% w/w, w/v or v/v to about 0.1% w/w, w/v or v/v of the silk fibroin fragments. In some embodiments, the silk fibroin protein fragment composition comprises from about 0.2% w/w, w/v or v/v to about 1.0% w/w, w/v or v/v of caprylyl/capryl glucoside and about 1.0% w/w, w/v or v/v to about 5.0% w/w, w/v or v/v of the silk fibroin fragments. In some embodiments, the silk fibroin protein fragment composition comprises from about 0.5% w/w, w/v or v/v to about 1.0% w/w, w/v or v/v of caprylyl/capryl glucoside and about 5.0% w/w, w/v or v/v to about 5.5% w/w, w/v or v/v of the silk fibroin fragments. In some embodiments, the silk fibroin protein fragment composition comprises from about 0.5 w/w of caprylyl/capryl glucoside and about 5.5% w/w of the silk fibroin fragments. In some embodiments, the silk fibroin protein fragment composition comprises from about 1.0% w/w of caprylyl/capryl glucoside and about 5.0 w/w of the silk fibroin fragments. In some embodiments, the silk fibroin protein fragment composition comprises the caprylyl/capryl glucoside and silk fibroin fragments in a weight ratio at a value selected from about 1:5 to about 1:11. In some embodiments, the silk fibroin protein fragment composition comprises caprylyl/capryl glucoside and silk fibroin fragments in a weight ratio of about 1:5. In some embodiments, the silk fibroin protein fragment composition comprises caprylyl/capryl glucoside and silk fibroin fragments in a weight ratio of about 1:11. In some embodiments, the silk fibroin protein fragment composition comprises caprylyl/capryl glucoside and silk fibroin fragments in a weight ratio of about 1:1. In some embodiments, the silk fibroin protein fragment composition comprises caprylyl/capryl glucoside and silk fibroin fragments in a weight ratio of about 1:2. In some embodiments, the silk fibroin protein fragment composition comprises caprylyl/capryl glucoside and silk fibroin fragments in a weight ratio of about 1:3. In some embodiments, the silk fibroin protein fragment composition comprises caprylyl/capryl glucoside and silk fibroin fragments in a weight ratio of about 1:4. In some embodiments, the silk fibroin protein fragment composition comprises caprylyl/capryl glucoside and silk fibroin fragments in a weight ratio of about 1:6. In some embodiments, the silk fibroin protein fragment composition comprises caprylyl/capryl glucoside and silk fibroin fragments in a weight ratio of about 1:7. In some embodiments, the silk fibroin protein fragment composition comprises caprylyl/capryl glucoside and silk fibroin fragments in a weight ratio of about 1:8. In some embodiments, the silk fibroin protein fragment composition comprises caprylyl/capryl glucoside and silk fibroin fragments in a weight ratio of about 1:9. In some embodiments, the silk fibroin protein fragment composition comprises caprylyl/capryl glucoside and silk fibroin fragments in a weight ratio of about 1:10. In some embodiments, the silk fibroin protein fragment composition comprises caprylyl/capryl glucoside and silk fibroin fragments in a weight ratio of about 1:12. In some embodiments, the silk fibroin protein fragment composition comprises caprylyl/capryl glucoside and silk fibroin fragments in a weight ratio of about 1:13. In some embodiments, the silk fibroin protein fragment composition comprises caprylyl/capryl glucoside and silk fibroin fragments in a weight ratio of about 1:14. In some embodiments, the silk fibroin protein fragment composition comprises caprylyl/capryl glucoside and silk fibroin fragments in a weight ratio of about 1:15.

In some embodiments, the silk emulsifier blend comprises silk fibroin protein fragments and a natural surfactant as co-surfactant. In some embodiments, the silk emulsifier blend may optionally comprises an additional protein/peptide emulsifier. In some embodiments, the silk emulsifier blend may optionally comprises a C12-C24 fatty alcohol. In some embodiments, the silk emulsifier blend may optionally comprises a glycolipid. In some embodiments, the silk emulsifier blend may optionally comprises a lipid.

In some embodiments, the natural surfactant is selected from the group consisting of protein, peptide, sugar surfactant, biosurfactant, lipid, and combinations thereof.

In some embodiments, a natural surfactant as co-emulsifier together with silk fibroin protein fragments form a synergistic emulsifier blend to reduce the surface tension of gas-water interface to greater than 50 mN/m at 20° C. as measured by standard surface tension apparatus and methods known to those of ordinary skill in the art, for example ASTM D1331-89 (2001) Method A, “Surface Tension”. Preferred synergistic emulsifier blends exhibit a minimum surface tension at water-gas interface of 30 mN/m or less. Suitable synergistic emulsifier blends promote stability of the oil in water emulsion by inhibiting coalescence of the oil droplets, and/or inhibiting phase separation of the oil and water phases.

In some embodiments, the natural surfactant in the synergistic emulsifier blend comprises sugar surfactants. In some embodiments, the sugar surfactant can be blends of different sugar fatty acid esters, such as sugar fatty acid monoesters, diesters, triesters, and polyesters. In some embodiments, the sugar surfactant is selected from the group consisting of sucrose fatty acid ester, sorbitan or sorbitol fatty acid ester, alkyl glucoside, alkyl polyglucoside, and combinations thereof. In some embodiments, the sugar surfactant is sucrose fatty acid ester. In some embodiments, the sugar surfactant is alkyl polyglucoside.

In some embodiments, the sugar surfactant has a HLB value greater than 8. In some embodiments, the sugar surfactant has a HLB value greater than 9.

In some embodiments, the sucrose fatty acid ester based co-emulsifier is added to the silk fibroin protein fragment composition to enhance silk fibroin protein fragment emulsification efficiency. In some embodiments, the sucrose fatty acid ester comprises sucrose fatty acid monoesters. In some embodiments, the natural surfactant may comprise a blend of sucrose esters. In some embodiments, the different sucrose fatty acid esters in the blend can vary in the length and/or saturation of the carbon chain of the fatty acid portion of the ester, or in the degree of esterification (e.g., whether the ester is a monoester, diester, triester, or polyester). Typically, the sucrose fatty acid ester surfactant comprises proportionally more monoesters than other types of esters (e.g., diesters, triesters, and polyesters).

In some embodiments, the sucrose fatty acid ester surfactants comprises a fatty acid chain having 12 to 18 carbon atoms (e.g., 12, 13, 14, 15, 16, 17, or 18 carbon atoms), such as stearic acid, lauric acid, oleic acid, and palmitic acid.

In some embodiments, the sucrose fatty acid ester surfactant has a HLB value ranging from 2 to 18. Typically, the lower the degree of esterification (e.g., average degree), the higher the HLB value of the sucrose fatty acid ester or mixture thereof. Exemplary HLB value for various sucrose esters include sucrose distearate (HLB=3), sucrose distearate/monostearate (HLB=12), sucrose dipalmitate (HLB=7.4); sucrose monostearate (HLB=15), sucrose monopalmitate (HLB>10), and sucrose monolaurate (HLB=15). In some embodiments, the sucrose ester has a HLB value ranging from about 14 to about 18. In some embodiments, the sucrose ester has a HLB value selected from the group consisting of about 14, about 15, about 16, about 17, about 18, about 19, and about 20. In some embodiments, the sucrose esters have an HLB value ranging from about 15 to about 18 (e.g., at or about 15, 16, 17, or 18).

In some embodiments, the sucrose ester is selected from the group consisting of sucrose cocoate, sucrose dilaurate, sucrose distearate, sucrose hexaerucate, sucrose laurate, sucrose myristate, sucrose oleate, sucrose palmitate, sucrose caprylate, sucrose decanoate, sucrose tridecanoate, sucrose undecanoate, sucrose pentadeconoate, sucrose heptadecanoate, sucrose pelargonate, sucrose pentaerucate, sucrose polybehenate, sucrose polycottonseedate, sucrose polylaurate, sucrose polylinoleate, sucrose polyoleate, sucrose polypalmate, sucrose polysoyate, sucrose polystearate, sucrose ricinoleate, sucrose stearate, sucrose tetraisostearate, sucrose tribehenate, sucrose tristearat, and combinations thereof. In some embodiments, the sucrose ester is selected from the group consisting of sucrose monostearate, sucrose monooleate, sucrose monopalmitate, sucrose monolaurate, and combinations thereof.

In some embodiments, the silk fibroin protein fragments composition is formed by mixing a sucrose fatty acid ester, the silk solution or the silk aqueous gel, and the hydrophobic emulsifiable component as described above, wherein the sucrose fatty acid ester is sucrose palmitate and/or sucrose laurate ester.

In some embodiments, the sucrose fatty acid esters may include commercial products sold under various trademark names, for example, DK Ester® F-160 (HLB=16, 1.23 degree of esterification, wt. % of Mono:Di:Tri-ester=72%:23%:5%), DK Ester® F-140 (HLB=13, 1.35 degree of esterification, wt. % of Mono:Di:Tri:poly-ester=61%:30%:7%:2%), DK Ester® F-110 (HLB=11, 1.48 degree of esterification, wt. % of Mono:Di:Tri:poly-ester=52%:36%:10%:2%), DK Ester® F-90 (HLB=9.5, 1.53 degree of esterification, wt. % of Mono:Di:Tri:poly-ester=45%:39%:12%:4%), DK Ester® F-70 (HLB=8, 1.60 degree of esterification, wt. % of Mono:Di:Tri:poly-ester=39%:45%:12%:4%), DK Ester® F-50 (HLB=6, 1.69 degree of esterification, wt. % of Mono:Di:Tri:poly-ester=34%:46%:17%:3%), DK Ester® F-20W (HLB=2, 3.11 degree of esterification, wt. % of Mono:Di:Tri:poly-ester=11%:21%:14%:54%), SURFHOPE® SE PHARMA J-1205 (HLB=5, 100% C12, 32 wt. % of Monoester and 68 wt. % Di:Tri:poly-ester), SURFHOPE® SE PHARMA J-1216 (HLB=16, 100% C12, 81 wt. % of Monoester and 19 wt. % Di:Tri:poly-ester), SURFHOPE® SE PHARMA J-1616 (HLB=16, 80% C16 and 20% C18, 79 wt. % of Monoester and 21 wt. % Di:Tri:poly-ester), SURFHOPE® SE PHARMA J-1805 (HLB=5, 70% C16 and 30% C18, 30 wt. % of Monoester and 70 wt. % Di:Tri:poly-ester), PHARMA J-1807 (HLB=7, 70% C16 and 30% C18, 41 wt. % of Monoester and 59 wt. % Di:Tri:poly-ester), SURFHOPE® SE PHARMA J-1816 (HLB=16, 70% C16 and 30% C18, 75 wt. % of Monoester and 25 wt. % Di:Tri:poly-ester), SURFHOPE® SE PHARMA D-1803 (HLB=3, sucrose stearate, 20 wt. % of Monoester and 80 wt. % Di:Tri:poly-ester), SURFHOPE® SE PHARMA D-1805 (HLB=5, sucrose stearate, 30 wt. % of Monoester and 70 wt. % Di:Tri:poly-ester), SURFHOPE® SE PHARMA D-1809 (HLB=7, sucrose stearate, 40 wt. % of Monoester and 60 wt. % Di:Tri:poly-ester), SURFHOPE® SE PHARMA D-1809 (HLB=9, sucrose stearate, 50 wt. % of Monoester and 50 wt. % Di:Tri:poly-ester), SURFHOPE® SE PHARMA D-1811 (HLB=11, sucrose stearate, 55 wt. % of Monoester and 45 wt. % Di:Tri:poly-ester), SURFHOPE® SE PHARMA D-1815 (HLB=15, sucrose stearate, 70 wt. % of Monoester and 30 wt. % Di:Tri:poly-ester), SURFHOPE® SE PHARMA D-1816 (HLB=16, sucrose stearate, 75 wt. % of Monoester and 25 wt. % Di:Tri:poly-ester), Ryoto S-970® (HLB=9, sucrose stearate, 50% monoester).

In some embodiments, a glucoside emulsifier having HLB value >10 is added to the silk fibroin protein fragment composition described herein to enhance silk fibroin protein emulsification efficiency. In some embodiments, the glucoside emulsifier is selected from the group consisting of alkyl polyglucoside having an alkyl group with 8 to 22 carbon atoms and a degree of glucoside unit condensation ranging from 1 to 7, alkyl polyglucoside having an alkyl group with 8 to 11 carbon atoms and a degree of glucoside unit condensation ranging from 1.0 to 1.4, alkyl polyglucoside having an alkyl group with 12 to 20 carbon atoms and a degree of glucoside unit condensation ranging from 1 to 7, alkyl polyglucoside having an alkyl group with 12 to 14 carbon atoms and a degree of glucoside unit condensation ranging from 1.5 to 4.0, methyl glycoside ester, ethyl glycoside esters, cetearyl glucoside, caprylyl/capryl glucoside, and combinations thereof. In some embodiments, the glucoside emulsifier is selected from the group consisting of cetearyl glucoside, caprylyl/capryl glucoside (APG C8-C10, e.g., a 63% aqueous solution of alkyl polyglucosides with 8-10 carbon alkyl chains and the average degree of polymerization DP=1.5), and combinations thereof. In some embodiments, the glucoside emulsifier is selected from the group consisting of octyl polyglucoside, 2-ethylhexyl polyglucoside, decyl polyglucoside, lauryl polyglucoside, myristyl polyglucoside, palmityl polyglucoside, isostearyl polyglucoside, stearyl polyglucoside, oleyl polyglucoside, behenyl polyglucoside, and combinations thereof. In some embodiments, the glucoside emulsifier is caprylyl/capryl glucoside.

In some embodiments, the synergistic emulsifier blend comprises a water-soluble glucoside containing an alkyl polyglucoside compound having alkyl chains with 6 to 14 carbons and degree of glucoside unit condensation ranging from 1.0 to 5.0. In some embodiments, the synergistic emulsifier blend comprises an oil soluble glucoside containing an alkyl polyglucoside compounds with alkyl chains having 16 to 22 carbon atoms. In general, increasing the degree of polymerization of the alkyl polyglucoside increases solubility in a polar medium, while lengthening of the alkyl chain increases solubility in a non-polar medium.

In some embodiments, the alkyl glucoside fatty ester based emulsifier is saponin. Saponins are natural alkyl glucoside surfactants consisting of molecules having one or more linear or branched hydrophilic glycoside moieties attached to a lipophilic triterpene or steroid aglycone (sapogenin). The saponins that are useful here is a triterpene glycoside. For example, saponins are found in soapwort plant (genus Saponaria), the root of which was used historically as a soap. The saponins are also found in soapbark tree, the inner bark of the soapbark tree can be reduced to powder and employed as a substitute for soap, since it forms a lather with water, owing to the presence of a glycoside saponin. They are amphipathic glycosides capable of producing soap-like foam when shaken in aqueous solutions.

In some embodiments, the saponin comprises at least one soya plant saponin component selected from the group consisting of a soya plant triterpenic saponin, a soya plant triterpenic sapogenol, and a soya plant extract containing at least one of said soya plant triterpenic saponin and of said soya plant triterpenic sapogenol. In some embodiments, the soya plant saponin component is extracted from a soya plant selected from the group consisting of Glycine max, Phaseolus vulgaris, Phaseolus aureus, Phaseolus lunatus, Vicia faba, Lens culinaris, Cicer arietum, Vigna angularis, Vigna mungo, Oxytropis ochrocephala, Oxytropis glabra, Pisum sativum, Sophora favescens, Asparalus membranaceus, Crotalaria albida, Arachis hypogea, Galega officinalis, Wistaria brachybotrys, and Trifolium repens, wherein the combination of the soya plant saponin component and the silk fibroin protein fragment composition are employed in a synergistic effective amount. The soya plant triterpenic saponin or sapogenol is reported as a cosmetic agent in skin care products for increasing the amount of collagen IV in the dermo-epidermal junction. The soya plant triterpenic saponin or sapogenol is a multifunctional cosmetic agent useful as silk fibroin protein fragment emulsifier co-emulsifier, and as skin care active agent. The silk fibroin protein and the soya plant triterpenic saponin or sapogenol act synergistically to increase collagen IV production in the skin and improve skin cosmetic appearance.

In some embodiments, the silk fibroin protein fragment composition comprises from about 0.05% w/w, w/v or v/v to about 8.0% w/w, w/v or v/v of glucoside emulsifier. In some embodiments, the silk fibroin protein fragment composition comprises from about 0.1% w/w, w/v or v/v to about 5.0% w/w, w/v or v/v of glucoside emulsifier. In some embodiments, the silk fibroin protein fragment composition comprises from about 0.3% w/w, w/v or v/v to about 1.0% w/w, w/v or v/v of glucoside emulsifier. In some embodiments, the silk fibroin protein fragment composition comprises a glucoside emulsifier at an weight percent selected from the group consisting of about 0.3%, 0.4%, 0.5%, 0.6%, 0.7%, 0.8%, 0.9% and 1.0% w/w, w/v or v/v.

In some embodiments, the glucoside emulsifier is present in an amount ranging from about 0.5% w/w, w/v or v/v to about 1.0% w/w, w/v or v/v and the silk fibroin protein fragment is present in an amount ranging from about 5.0% w/w, w/v or v/v to about 5.5 w/w, w/v or v/v by the basis of the silk fibroin fragment composition. In some embodiments, the glucoside emulsifier is present in an amount of about 0.5 w/w and the silk fibroin protein fragment is present in an amount of about 5.5 w/w by the basis of the silk fibroin fragment composition. In some embodiments, the glucoside emulsifier is present in an amount of about 1% w/w and the silk fibroin protein fragment is present in an amount of about 5.0 w/w by the basis of the silk fibroin fragment composition.

In some embodiments, the glucoside emulsifier has a weight ratio of the glucoside emulsifier to the silk fibroin fragments at a value ranging from 1:4 to 1:20. In some embodiments, the glucoside emulsifier has a weight ratio of the glucoside emulsifier to the silk fibroin fragments at a value selected from the group consisting of 1:4, 1:5, 1:6, 1:7, 1:8, 1:9, 1:10, 1:11, 1:12, 1:13, 1:14, 1:15, 1:16, 1:17, 1:18, 1:19 and 1:20. In some embodiments, the glucoside emulsifier has a weight ratio of the glucoside emulsifier to the silk fibroin fragments at a value selected from about 1:5 to about 1:11. In some embodiments, the glucoside emulsifier has a weight ratio of the glucoside emulsifier to the silk fibroin fragments of about 1:5. In some embodiments, the glucoside emulsifier has a weight ratio of the glucoside emulsifier to the silk fibroin fragments of about 1:11.

In some embodiments, the glucoside emulsifier is caprylyl/capryl glucoside. In some embodiments, the silk fibroin protein fragment composition comprises from about 0.2% w/w, w/v or v/v to about 1.0% w/w, w/v or v/v of caprylyl/capryl glucoside and about 1.0% w/w, w/v or v/v to about 5.0% w/w, w/v or v/v of the silk fibroin fragments. In some embodiments, the silk fibroin protein fragment composition comprises from about 0.5 w/w, w/v or v/v to about 1.0% w/w, w/v or v/v of caprylyl/capryl glucoside and about 5.0% w/w, w/v or v/v to about 5.5% w/w, w/v or v/v of the silk fibroin fragments. In some embodiments, the silk fibroin protein fragment composition comprises from about 0.5% w/w of caprylyl/capryl glucoside and about 5.5% w/w of the silk fibroin fragments. In some embodiments, the silk fibroin protein fragment composition comprises from about 1.0% w/w of caprylyl/capryl glucoside and about 5.0% w/w of the silk fibroin fragments. In some embodiments, the silk fibroin protein fragment composition comprises the caprylyl/capryl glucoside and silk fibroin fragments in a weight ratio at a value selected from about 1:5 to about 1:11. In some embodiments, the silk fibroin protein fragment composition comprises caprylyl/capryl glucoside and silk fibroin fragments in a weight ratio of about 1:5. In some embodiments, the silk fibroin protein fragment composition comprises caprylyl/capryl glucoside and silk fibroin fragments in a weight ratio of about 1:11.

In some embodiments, a fatty acid sorbitan ester or sorbitol ester is added to the silk fibroin protein fragment composition as co-emulsifier to enhance silk fibroin protein emulsification efficiency. Sorbitan ester emulsifier is prepared by the reaction of sorbitol with fatty acids or derivatives thereof, and results in a complex mixture of products including sorbitol mono- di-, tri-, and higher esters, sorbitan mono-, di-, and higher-esters, isosorbide mono-, and di-esters, and non-esterified sorbitol, sorbitan and isosorbide.

In some embodiments, the sorbitan ester is selected from the group consisting of sorbitan fatty acid esters having C10-20 fatty acid, polyoxyethylene sorbitan fatty acid esters having C10-20 fatty acid, and combinations thereof. In some embodiments, the sorbitan ester is sorbitan stearate, sorbitan isostearate, sorbitan palmitate, sorbitan monolaurate (TEGO® SML, Evonik), sorbitan monooleate, sorbitan sesquicaprylate (ANTIL® Soft SC Evonik), sorbitol laurate, sorbitan cocoate, sorbitan caprylate, sorbitan caprylate, sorbitan myristate, sorbitan octanoate, sorbitan 2-ethylhexanoate, sorbitan behenate, and combinations thereof. In some embodiments, the sorbitan ester is selected from the group consisting of sorbitan stearate, sorbitan palmitate, sorbitan laurate, and combinations thereof. In some embodiments, the synergistic emulsifier blend comprises a mixture of silk fibroin protein fragments as described above and one or more of sorbitan monostearate and sorbitan monooleate.

In some embodiments, the sorbitan ester is present in an amount less than 5.0% w/w, w/v or v/v by the basis of the silk fibroin protein fragment composition. In some embodiments, the sorbitan ester is present in an amount less than 3.0% w/w, w/v or v/v. In some embodiments, the sorbitan ester is present in an amount ranging from about 0.2% w/w, w/v or v/v to about 2.0% w/w, w/v or v/v by the total weight of the silk fibroin protein fragment composition. In some embodiments, the sorbitan ester is present in an amount ranging from about 0.5% w/w, w/v or v/v to about 1.5 w/w, w/v or v/v by the total weight of the silk fibroin protein fragment composition.

In some embodiments, the silk fibroin protein fragment composition comprises from about 0.2% w/w, w/v or v/v to about 2.0% w/w, w/v or v/v of sorbitan ester and about 1.0% w/w, w/v or v/v to about 5.0% w/w, w/v or v/v of silk fibroin protein fragments. In some embodiments, the silk fibroin protein fragment composition comprises from about 1.0% w/w, w/v or v/v of sorbitan ester and about 5.0% w/w, w/v or v/v of silk fibroin protein fragments.

In some embodiments, an acyl N-methylglucamine is added to the silk fibroin protein fragment composition as emulsion stabilizer to enhance silk fibroin protein emulsification efficiency. In some embodiment, the acyl N-methylglucamine has an acyl group selected from the group consisting of C18-24 acyl group, acyl group derived from palmitic acid (C16:0), acyl group derived from stearic acid (C18:0), acyl group derived from oleic acid (C18:1), and acyl group derived from linoleic acid.

In some embodiments, the silk fibroin protein fragment composition comprises from about 0.2% w/w, w/v or v/v to about 1.0% w/w, w/v or v/v of acyl N-methylglucamine and about 1.0% w/w, w/v or v/v to about 5.0% w/w, w/v or v/v of the silk fibroin fragments. In some embodiments, the silk fibroin protein fragment composition comprises from about 1.0% w/w, w/v or v/v of acyl N-methylglucamine and about 5.0% w/w, w/v or v/v of the silk fibroin fragments.

In some embodiments, the silk fibroin protein fragment composition comprises from about 0.2% w/w, w/v or v/v to about 1.0% w/w, w/v or v/v of caprylyl/capryl glucoside and about 1.0% w/w, w/v or v/v to about 5.5% w/w, w/v or v/v of the silk fibroin fragments. In some embodiments, the silk fibroin protein fragment composition comprises from about 0.5% w/w, w/v or v/v of caprylyl/capryl glucoside and about 5.5% w/w, w/v or v/v of the silk fibroin fragments. In some embodiments, the silk fibroin protein fragment composition comprises from about 1.0% w/w, w/v or v/v of caprylyl/capryl glucoside and about 5.0% w/w, w/v or v/v of the silk fibroin fragments.

In some embodiments, the silk fibroin protein fragment composition comprises from about 0.2% w/w, w/v or v/v to about 1.0% w/w, w/v or v/v of caprylyl/capryl glucoside and about 1.0% w/w, w/v or v/v to about 5.0 w/w, w/v or v/v of the silk fibroin fragments. In some embodiments, the silk fibroin protein fragment composition comprises from about 0.5% w/w, w/v or v/v to about 1.0% w/w, w/v or v/v of caprylyl/capryl glucoside and about 5.0% w/w, w/v or v/v to about 5.5 w/w, w/v or v/v of the silk fibroin fragments. In some embodiments, the silk fibroin protein fragment composition comprises from about 0.5 w/w of caprylyl/capryl glucoside and about 5.5 w/w of the silk fibroin fragments. In some embodiments, the silk fibroin protein fragment composition comprises from about 1.0% w/w of caprylyl/capryl glucoside and about 5.0 w/w of the silk fibroin fragments. In some embodiments, the silk fibroin protein fragment composition comprises the caprylyl/capryl glucoside and silk fibroin fragments in a weight ratio at a value selected from about 1:5 to about 1:11. In some embodiments, the silk fibroin protein fragment composition comprises caprylyl/capryl glucoside and silk fibroin fragments in a weight ratio of about 1:5. In some embodiments, the silk fibroin protein fragment composition comprises caprylyl/capryl glucoside and silk fibroin fragments in a weight ratio of about 1:11.

In some embodiments, a glycolipid is added to the silk fibroin protein fragment composition as emulsion stabilizer to enhance silk fibroin protein emulsification efficiency. In some embodiments, the emulsifier system for the silk fibroin protein fragment composition comprises a blend of silk fibroin protein fragments and one or more selected form the group consisting of SLES, CAPB, rhamnolipids, sophorolipids to stabilize an emulsion at pH ranging from 4.5 to 9.0. In some embodiments, the natural surfactant comprises the glycolipid selected from the group consisting of rhamnolipid, monorhamnolipid, dirhamnolipid, sophorolipid, lactonic sophorolipid, trehalolipid, mannosylerythritol lipid (ustilipid), and combinations thereof.

In some embodiments, the silk fibroin protein fragment composition comprises less than 3.0% w/w, w/v or v/v of the glycolipid and about 1.0% w/w, w/v or v/v to about 5.0% w/w, w/v or v/v of the silk fibroin fragments. In some embodiments, the silk fibroin protein fragment composition comprises about 0.2% w/w, w/v or v/v to about 2.0% w/w, w/v or v/v of the glycolipid and about 1.0% w/w, w/v or v/v to about 5.0% w/w, w/v or v/v of the silk fibroin fragments. In some embodiments, the silk fibroin protein fragment composition comprises about 0.5% w/w, w/v or v/v to about 1.5% w/w, w/v or v/v of the glycolipid and about 1.0% w/w, w/v or v/v to about 5.0% w/w, w/v or v/v of the silk fibroin fragments. In some embodiments, the silk fibroin protein fragment composition comprises from about 1.0% w/w, w/v or v/v of glycolipid and about 5.0% w/w, w/v or v/v of the silk fibroin fragments.

In some embodiments, the natural surfactants are present in an amount ranging from about 0.001% w/w, w/v or v/v to about 2.0% w/w, w/v or v/v by the basis of the silk fibroin fragment composition. In some embodiments, the natural surfactants are present in an amount ranging from about 0.01% w/w, w/v or v/v to 2.0% w/w, w/v or v/v. In some embodiments, the natural surfactant has a weight ratio of the natural surfactant to the silk fibroin fragments at a value ranging from 1:4 to 1:20. In some embodiments, the natural surfactant has a weight ratio of the natural surfactant to the silk fibroin fragments at a value selected from the group consisting of 1:4, 1:5, 1:6, 1:7, 1:8, 1:9, 1:10, 1:11, 1:12, 1:13, 1:14, 1:15, 1:16, 1:17, 1:18, 1:19 and 1:20. In some embodiments, the natural surfactant has a weight ratio of the natural surfactant to the silk fibroin fragments at a value selected from about 1:5 to about 1:11. In some embodiments, the natural surfactant has a weight ratio of the natural surfactant to the silk fibroin fragments at a value selected from about 1:5. In some embodiments, the natural surfactant has a weight ratio of the natural surfactant to the silk fibroin fragments at a value selected from about 1:11.

In some embodiments, the silk fibroin fragment composition comprises silk fibroin fragments and a natural surfactant, wherein the weight ratio of the natural surfactant to the silk fibroin fragments is a value selected from the group consisting of 1:4, 1:5, 1:6, 1:7, 1:8, 1:9, 1:10, 1:11, 1:12, 1:13, 1:14, 1:15, 1:16, 1:17, 1:18, 1:19 and 1:20. In some embodiments, the silk fibroin fragment composition comprises silk fibroin fragments and a natural surfactant, wherein weight ratio of the natural surfactant to the silk fibroin fragments is a value selected from about 1:5 to about 1:11. In some embodiments, the silk fibroin fragment composition comprises silk fibroin fragments and a natural surfactant, wherein the weight ratio of the natural surfactant to the silk fibroin fragments is about 1:5. In some embodiments, the silk fibroin fragment composition comprises silk fibroin fragments and a natural surfactant, wherein the weight ratio of the natural surfactant to the silk fibroin fragments is about 1:11.

In some embodiments, the natural surfactant is present in an amount ranging from about 0.2% w/w, w/v or v/v to about 1.0% w/w, w/v or v/v of and the silk fibroin protein fragment is present in an amount ranging from about 1.0% w/w, w/v or v/v to about 5.0% w/w, w/v or v/v by the basis of the silk fibroin fragment composition. In some embodiments, the natural surfacing is present in an amount of about 1.0% w/w, w/v or v/v and silk fibroin protein fragment is present in an amount of about 5.0% w/w, w/v or v/v by the basis the silk fibroin fragment composition. In some embodiments, the natural surfactant is present in an amount ranging from about 0.5% w/w, w/v or v/v to about 1.0% w/w, w/v or v/v of and the silk fibroin protein fragment is present in an amount ranging from about 5.0% w/w, w/v or v/v to about 5.5% w/w, w/v or v/v by the basis of the silk fibroin fragment composition. In some embodiments, the natural surfactant is present in an amount of about 0.5% w/w and the silk fibroin protein fragment is present in an amount of about 5.5% w/w by the basis of the silk fibroin fragment composition. In some embodiments, the natural surfactant is present in an amount of about 1% w/w and the silk fibroin protein fragment is present in an amount of about 5.0 w/w by the basis of the silk fibroin fragment composition.

In some embodiments, the natural surfactant is caprylyl/capryl glucoside. In some embodiments, the silk fibroin protein fragment composition comprises from about 0.2% w/w, w/v or v/v to about 1.0% w/w, w/v or v/v of caprylyl/capryl glucoside and about 1.0% w/w, w/v or v/v to about 5.0% w/w, w/v or v/v of the silk fibroin fragments. In some embodiments, the silk fibroin protein fragment composition comprises from about 0.5% w/w, w/v or v/v to about 1.0% w/w, w/v or v/v of caprylyl/capryl glucoside and about 5.0% w/w, w/v or v/v to about 5.5% w/w, w/v or v/v of the silk fibroin fragments. In some embodiments, the silk fibroin protein fragment composition comprises from about 0.5 w/w of caprylyl/capryl glucoside and about 5.5% w/w of the silk fibroin fragments. In some embodiments, the silk fibroin protein fragment composition comprises from about 1.0% w/w of caprylyl/capryl glucoside and about 5.0 w/w of the silk fibroin fragments. In some embodiments, the silk fibroin protein fragment composition comprises caprylyl/capryl glucoside and silk fibroin fragments in a weight ratio at a value selected about 1:5 to about 1:11. In some embodiments, the silk fibroin protein fragment composition comprises caprylyl/capryl glucoside and silk fibroin fragments in a weight ratio of about 1:5. In some embodiments, the silk fibroin protein fragment composition comprises caprylyl/capryl glucoside and silk fibroin fragments in a weight ratio of about 1:11.

In some embodiments, the natural surfactant comprises the biosurfactant selected from the group consisting of glycolipids, fatty acid, neutral lipid, phospholipids, polymeric biosurfactants, lipopeptides (surfactin, iturin, fengycin, lichenysin), and combinations thereof.

In some embodiments, the silk fibroin protein fragments used as emulsifier has a weight average molecular weight of greater than about 5 kDa. In some embodiments, the silk fibroin protein used as emulsifier has a weight average molecular weight ranging from about 5 kDa to about 350 kDa. In some embodiments, the silk fibroin protein used as emulsifier has a weight average molecular weight ranging from about 20 kDa to about 80 kDa. In some embodiments, the silk fibroin protein used as emulsifier has a weight average molecular weight ranging from about 40 kDa to about 60 kDa. In other embodiments, any silk fibroin fragments described herein can be used as emulsifiers.

In some embodiments, the silk fibroin protein fragments composition comprises about 0.1% w/w, w/v or v/v to about 15.0% w/w, w/v or v/v of the synergistic emulsifier blend. In some embodiments, the silk fibroin protein fragments composition comprises about 0.75% w/w, w/v or v/v to about 10.0% w/w, w/v or v/v of the synergistic emulsifier blend. In some embodiments, the silk fibroin protein fragments composition comprises the synergistic emulsifier blend at an amount selected from the group consisting of about 0.1%, about 0.2%, about 0.3%, about 0.4%, about 0.5%, about 0.6%, about 0.7%, about 0.8%, about 0.9%, about 1.0%, about 1.25%, about 1.50%, about 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, about 5.0%, about 5.25%, about 5.5%, about 5.75%, about 6.0%, about 6.25%, about 7.5%, about 7.75%, about 8.0%, about 8.25%, about 8.5%, about 8.75%, about 9.0%, about 9.25%, about 9.5%, about 9.75%, about 10.0%, about 10.25%, about 10.5%, about 10.75%, about 11.0%, about 11.25%, about 11.5%, about 11.75%, about 12.0%, about 12.25%, about 12.50%, about 12.75%, about 13.0%, about 13.25%, about 13.50%, about 13.75%, about 14.0%, about 14.25%, about 14.50%, about 14.75%, and about 15.0% w/w, w/v or v/v by the basis the silk fibroin fragment composition.

In some embodiments, the emulsifiable component comprises a hydrophobic emulsifiable component, a hydrophilic emulsifiable component, or both. In an embodiment, the aqueous solution of silk fibroin protein fragments as described above may be admixed with the emulsifiable component to achieve uniform emulsification. In an embodiment, an aqueous gel of the silk fibroin protein fragments may be mixed with the emulsifiable component to achieve uniform emulsification.

In some embodiments, the emulsifiable component comprises a hydrophobic emulsifiable component. In some embodiments, the hydrophobic emulsifiable component is selected from the group consisting of oil, fat, wax, lipid, and combinations thereof.

In some embodiments, the oil in the silk fibroin fragment composition is selected from the group consisting of hydrocarbon oils, higher fatty acids, higher alcohols, synthetic ester oils, glyceride fatty esters, glyceryl trioctanoate, glyceryl triisopalmitate, cholesteryl isostearate, isopropyl palmitate, isopropyl myristate, neopentylglycol dicaprate, isopropyl isostearate, octadecyl myristate, cetyl 2-ethylhexanoate, cetearyl isononanoate, cetearyl octanoate, isononyl isononanoate, isotridecyl isononanoate, glyceryl tri-2-ethylhexanoate, glyceryl tri(caprylatelcaprate), diethylene glycol monoethyl ether oleate, dicaprylyl ether, caprylic acid/capric acid propylene glycol diester, isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexylate, dipentaerythritol fatty acid ester, N-alkyl glycol monoisostearate, neopentyl glycol dicaprate, diisostearyl malate, glyceryl di-2-heptylundecanoate, trimethylolpropane tri-2-ethylhexylate, trimethylolpropane triisostearate, pentaneerythritol tetra-2-ethylhexylate, glyceryl tri-2-ethylhexylate, trimethylolpropane triisostearate, cetyl 2-ethylhexanoate, 2-ethylhexyl palmitate, glyceryl trimyristate, tri-2-heptylundecanoic glyceride, castor oil fatty acid methyl ester, oleyl oleate, cetostearyl alcohol, acetoglyceride, 2-heptylundecyl palmitate, diisopropyl adipate, N-lauroyl-L-glutamic acid-2-octyldodecyl ester, di-2-heptylundecyl adipate, ethyl laurate, di-2-ethylhexyl cebatate. 2-hexyldecyl myristate, 2-hexyldecyl palmitate, 2-hexyldecyl adipate, diisopropyl cebatate, 2-ethylhexyl succinate, ethyl acetate, butyl acetate, amyl acetate and triethyl |citrate, mineral oil, light mineral oil, squalane, paraffin oil, silicone oil, lauric acid, myristic acid, stearic acid, lauryl alcohol, myristyl alcohol, palmityl alcohol, stearyl alcohol, cetyl alcohol, and combinations thereof. In some embodiments, the oil in the silk fibroin fragment composition comprises hydrocarbon oil selected from the group consisting of liquid petrolatum, squalane, squalene, pristane, paraffin, isoparaffin, ceresin, squalene, mineral oil, light mineral oil, blend of light mineral oil and heavy mineral oil, polyisobutene, hydrogenated polyisobutene, terpene oil, and combinations thereof. In some embodiments, the oil in the silk fibroin fragment composition comprises squalane or terpene oil.

In some embodiments, the hydrocarbon oil is present in the silk fibroin protein fragments composition at an amount of about 80.0% w/w, w/v or v/v by the basis of the silk fibroin protein fragments composition. In some embodiments, the hydrocarbon oil is present in the silk fibroin protein fragments composition at an amount selected from about 20.0%, about 30.0%, about 40.0%, about 50.0%, about 60.0%, about 70.0%, about 80.0% w/w, w/v or v/v by the basis of the silk fibroin protein fragments composition.

In some embodiments, the fat in the silk fibroin fragment composition is selected from the group consisting of liquid fats, solid fats, avocado oil, tsubaki oil, turtle oil, macadamia nut oil, corn oil, mink oil, olive oil, rape seed oil, egg yolk oil, sesame seed oil, persic oil, wheat germ oil, sasanqua oil, castor oil, linseed oil, safflower oil, cotton seed oil, perilla oil, soybean oil, peanut oil, tea seed oil, kaya oil, rice bran oil, Chinese wood oil, Japanese wood oil, jojoba oil, germ oil, sweet almond oil, rosehip seed oil, calendula oil, grape seed oil, apricot kernel oil, flaxseed oil, hazelnut oil, walnut oil, pecan nut oil, macadamia nut oil, sesame oil, emu oil, coconut oil, sunflower oil, canola oil, soybean oil, algae oil, cacao butter, coconut oil, horse tallow, hardened coconut oil, palm oil, beef tallow, sheep tallow, hardened beef tallow, palm kernel oil, pork tallow, beef bone tallow, Japanese core wax, hardened oil, neatsfoot tallow, Japanese wax, hydrogenated castor oil, synthetic ester oil derived from the condensation product of long chain mono unsaturated acid having 16 to 24 carbon atoms and fatty alcohol having 16 to 26 carbon atoms, ester oil of oleic acid and erucic acid with oleic alcohol or erucyl alcohol, and combinations thereof. In some embodiments, the fat in the silk fibroin fragment composition comprises soybean oil and olive oil. In some embodiments, the fat in the silk fibroin protein fragment composition comprise jojoba oil selected from the group consisting of natural jojoba oil, partially hydrogenated natural or synthetic jojoba oil, completely hydrogenated natural or synthetic jojoba oil, and isomerized natural or synthetic jojoba oil. In some embodiments, the fat in the silk fibroin protein fragment composition comprise ester oil of oleic acid and erucic acid with oleic alcohol or erucyl alcohol.

In some embodiments, jojoba oil is present in the silk fibroin protein fragments composition at an amount of about 80% w/w, w/v or v/v by the silk fibroin protein fragments composition. In some embodiments, jojoba oil is present in the silk fibroin protein fragments composition at an amount selected from about 20%, about 30%, about 40%, about 50%, about 60%, about 70%, about 80% w/w, w/v or v/v by the basis of the silk fibroin protein fragments composition.

In some embodiments, the wax in the silk fibroin fragment composition is selected from the group consisting of butter, petrolatum, polyethylene wax, polypropylene wax, beeswax, candelilla wax, paraffin wax, ozokerite, microcrystalline waxes, carnauba wax, cotton wax, esparto wax, bayberry wax, tree wax, whale wax, montan wax, bran wax, lanolin, kapok wax, lanolin acetate, liquid lanolin, sugar cane wax, lanolin fatty acid isopropyl ester, hexyl laurate, reduced lanolin, jojoba wax, hard lanolin, shellac wax, POE lanolin alcohol ether, POE lanolin alcohol acetate, POE cholesterol ether, lanolin fatty acid polyethylene glycol, POE hydrogenated lanolin alcohol ether.

In some embodiments, the lipid is selected from the group consisting of phospholipids, polymer-lipid conjugate, carbohydrate-lipid conjugate, dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-dimyristoyl-sn-glycero-3-phosphorylglycerol (DMPG); 1,2-distearoyl-sn-glycero-3-phosphoethanolamine (DSPE); 1,2-dioleoyl-sn-glycero-3-phosphocholine (DOPC); 1,2-dioleoyl-sn-glycero-3-phosphoethanolamine (DOPE); 1,2-dipalmitoyl-sn-glycero-3-phospho-(1′-rac-glycerol) (DPPG); 1,2-distearoyl-sn-glycero-3-phosphocholine (DSPC), distearoylphosphoethanolamine conjugated with polyethylene glycol (DSPE-PEG); phosphatidylserine (PS), phosphatidylethanolamine (PE), phosphatidylglycerol (PG), phosphatidylcholine (PC), and combinations thereof. In an embodiment, the particle comprise the lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, PG, 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-diarachidyl-sn-glycero-3-phosphoethanolamine (20:0 PE), 1-stearoyl-2-linoleoyl-sn-glycero-3-phosphoethanolamine, 1,2-diheptadecanoyl-sn-glycero-3-phosphocholine (17:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC). In some embodiments, the phospholipid is selected from the group consisting of soy lecithin and egg lecithin.

In some embodiments, the hydrophobic emulsifiable component is selected from the group consisting of jojoba oil, squalane, liquid paraffin, liquid isoparaffin, neopentylglycol dicaprate, isopropyl isostearate, cetyl 2-ethylhesanoate, isononyl isononanoate, glyceryl tri(caprylatelcaprate), methyl polysiloxane having a molecular weight ranging from 100 to 500, decamethylcydopentasiloxane, octamethylcydotetrasiloxane, higher fatty acids having a carbon number ranging from 12 to 22, higher alcohols having a carbon number ranging from 12 to 22, ceramides, glycolipids, terpene oil, and combinations thereof. In some embodiments, the hydrophobic emulsifiable component is selected from the group consisting of jojoba oil, squalane, isononyl isononanoate, glyceryl tri(caprylatelcaprate), and combinations thereof. In some embodiments, the hydrophobic emulsifiable component comprises jojoba oil and/or squalane.

In some embodiments, the silk fibroin protein fragments composition comprises about 20% to about 80% w/v, w/w or v/v of the emulsifiable component. In some embodiments, the silk fibroin protein fragments composition comprises about 80% w/v, w/w or v/v of the emulsifiable component. In some embodiments, In some embodiments, the emulsifiable component has a weight percent selected from the group consisting of about 10.0%, about 11.0%, about 12.0%, about 13.0%, about 14.0%, about 15.0%, about 16.0%, about 17.0%, about 18.0%, about 19.0%, about 20.0%, about 21.0%, about 22.0%, about 23.0%, about 24.0%, about 25.0%, about 26.0%, about 27.0%, about 28.0%, about 29.0%, about 30%, about 31.0%, about 32.0%, about 33.0%, about 34.0%, about 35%, about 36.0%, about 37.0%, about 38.0%, about 39.0%, about 40%, about 41.0%, about 42.0%, about 43.0%, about 44.0, about 45%, about 46.0%, about 47.0%, about 48.0%, about 49.0%, about 50%, about 55%, about 56.0%, about 57.0%, about 58.0%, about 59.0%, about 60%, about 61.0%, about 62.0%, about 63.0%, about 64.0%, about 65%, about 66.0%, about 67.0%, about 68.0%, about 69.0%, about 70%, about 71.0%, about 72.0%, about 73.0%, about 74.0%, about 75%, about 76.0%, about 77.0%, about 78.0%, about 79.0%, about 80%, about 81.0%, about 82.0%, about 83.0%, about 84.0%, about 85%, about 86.0%, about 87.0%, about 88.0%, about 89.0%, about 90%, about 91.0%, about 92.0%, about 93.0%, about 94.0%, about 95%, about 96.0%, about 97.0%, about 98.0%, about 99.0%, and about 99% w/v, w/w or v/v by the basis of the silk fibroin protein fragments composition.

Silk protein in the aqueous solution tends to fibrillate more readily by shear of vibration or stirring if it has a higher molecular weight (e.g., Mw greater than 100 kDa). The water-insoluble masses of the fibrillated protein causes reduction of pleasant feel during use of the cosmetic materials.

In some embodiments, the silk fibroin protein fragments are blended with hydrophilic substance with high HLB value to enhance the hydrophilic environment and to prevent silk fibroin protein fragments composition from gelation. It is important to prevent fibroin transformation from random coils to β-sheet structure (fibrillate).

In some embodiments, the hydrophilic substance is selected from the group consisting of propanediol, ethanediol, glycerol, butantetraol, xylitol, cyclodextrin, α-cyclodextrin, β-cyclodextrin, γ-cyclodextrin, D-sorbitol, inositol polyethylene glycol, polyethylene oxide, polylactic acid, cellulose, chitin, polyvinyl alcohol, and combinations thereof. In some embodiments, the hydrophilic substance is glycerol. In some embodiments, the hydrophilic substance is cyclodextrin.

In some embodiments, the silk fibroin protein fragments composition comprises the hydrophilic substance at a weight percent ranging from about 0.5 wt. % to about 10.0 wt. %. In some embodiments, the silk fibroin protein fragments composition comprises the hydrophilic substance at a weight percent ranging from about 0.5 wt. % to about 5.0 wt. %. In some embodiments, the silk fibroin protein fragments composition comprises the hydrophilic substance at a weight percent ranging from about 0.5 wt. % to about 3.0 wt. %. In some embodiments, the silk fibroin protein fragments composition comprises the hydrophilic substance at a weight percent ranging from about 0.5 wt. % to about 1.0 wt. %. In some embodiments, the silk fibroin protein fragments composition comprises the hydrophilic substance at a weight percent selected from the group consisting of about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7.0 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8.0 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9.0 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, and about 10.0 wt. %,

In some embodiments, the silk fibroin protein fragment composition comprises the hydrophilic substance at a weight ratio of the hydrophilic molecule to the silk fibroin protein fragments of 1:1 to 1:10. In some embodiments, the weight ratio of the hydrophilic molecule to the silk fibroin protein fragments is selected from the group consisting of 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5.0, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:8, 1:9, and 1:10. In some embodiments, the weight ratio of the hydrophilic molecule to the silk fibroin protein fragments is 1:1. In some embodiments, silk fibroin protein fragment composition comprises glycerol at a weight ratio of glycerol to the silk fibroin protein fragments of 1:1 to 1:3. In some embodiments, the a weight ratio of glycerol to the silk fibroin protein fragments is selected from the group consisting of 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, and 1:3.0.

In some embodiments, the silk fibroin fragment composition further comprises a thickening agent or gelling agent selected from the group consisting of hydroxyethyl cellulose, hydroxypropyl methylcellulose, cyclodextrin, dextran, gelatin, carboxymethyl cellulose, propylene glycol, polyethylene glycol, polysorbate 80, polyvinyl alcohol, povidone, sucrose, fructose, maltose, carrageenan, chitosan, alginate, hyaluronic acid, gum arabic, galactomannans, pectin, and combinations thereof. Without the thickening agent, O/W emulsions are unstable to creaming once radius of the emulsion droplets is greater than 0.5 μm. In some embodiments, the thickening/gelling agent comprises carrageenan. In some embodiments, the thickening/gelling agent comprises xanthan gum.

Xanthan gum is used throughout the cosmetic, pharmaceutical and agricultural industries because of its ability to stabilize emulsions and act as a dispersing agent due to its ability to thicken aqueous solutions. Xanthan gum has various applications due to its rheological properties that has led to its industrial success. Xanthan gum was added to surfactants to improve viscosity and other rheological properties because surfactants elevating oxygen transfer.

Polysaccharides and surfactants are typically added into emulsions to improve the emulsion systems stability. Surfactants are added to improve the formulations interfacial properties while the polysaccharides are added to improve its rheological performance, specifically its viscoelastic properties. Carrageenan is a natural polysaccharides derived from seaweed. Carrageenan is applied to emulsion to increase viscosity and induce gelling. Carrageenan's ability for stabilizing formulations is due to thickening and gelling properties. Gel and surfactant and surfactant mixtures show either cubic system, lamellar ordering or hexagonal arrangement. Due to the hydrophobic interactions of the surfactant used and the polymeric gel and surfactants electrostatic interaction the ordered structures are formed. The mixture of κ-carrageenan gel and an ionic surfactant also forms an ordered structure. The polymer network and surfactant system cause the ordering by way of hydrophobic and electrostatic interactions. The type of ordering that is formed from these interactions is the lamellar type, self-assembled by the carrageenan molecules.

In some embodiments, the synergistic surfactant blend of silk fibroin protein fragments and sugar surfactant described above further combined with a thickening/gelling agent selected from the group consisting of carrageenan, xanthan gum, and combinations thereof. In some embodiments, the blend preferably has 5.5 wt. % silk fibroin protein fragments and 0.5 wt. % glucoside and the blend attains low surface tension of 26.48 mN/m. Carrageenan and xanthan gum slightly increase the surface tension of the aqueous solution of the synergistic surfactant blend to 27.5 mN/m and 29.73 mN/m respectively with 0.1 grams of thickener added to a 20 ml of surfactant solution. Despite the slight increase of surface tension, there is barely a change in the mixtures foaming capabilities when the thickeners were added. Both carrageenan and xanthan gum significantly increase the viscosity of the sample. However, carrageenan increased viscosity more than xanthan gum. The aqueous solution of synergistic surfactant blend of 5.5 wt. % silk fibroin protein fragments and 0.5 wt. % glucoside without any thickener has a viscosity of 0.0027 Pa·s, whereas the addition of a small amount of carrageenan and xanthan gum, as low as 0.1 gram to a 20 mL aqueous solution of the surfactant blend increased the viscosity to 3.08 Pa·s for carrageenan and 3.58 Pa·s for xanthan gum.

In some embodiments, the silk fibroin fragment composition comprises about 0.01 wt. % to about 10.0 wt. % of the thickening/gelling agent. In some embodiments, the silk fibroin fragment composition comprises about 0.2 wt. % to about 2.0 wt. % of the thickening/gelling agent. In some embodiments, the silk fibroin fragment composition comprises the thickening/gelling agent at an amount selected from the group consisting of about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7.0 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8.0 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9.0 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, and about 10.0 wt. % by the basis of the silk fibroin fragment composition. In some embodiments, the silk fibroin fragment composition comprises the thickening/gelling agent at about 0.5 wt. % by the basis of the silk fibroin fragment composition.

In some embodiments, the thickening/gelling agent is hyaluronic acid at about 0.2 wt. % by the total weight of the silk fibroin fragment composition. In some embodiments, the thickening/gelling agent is xanthan gum at about 0.5 wt. % by the total weight of the silk fibroin fragment composition. In some embodiments, the thickening/gelling agent is carrageenan at about 0.5 wt. % by the total weight of the silk fibroin fragment composition.

In some embodiments, the silk fibroin fragment composition comprises silk fibroin fragments, a natural surfactant, and a thickening agent. In some embodiments, the weight ratio of the natural surfactant to the thickening/gelling agent to the silk fibroin fragments is a value selected from the group consisting of 1:1:4, 1:1:5, 1:1:6, 1:1:7, 1:1:8, 1:9, 1:10, 1:1:11, 1:1:12, 1:1:13, 1:1:14, 1:1:15, 1:1:16, 1:1:17, 1:1:18, 1:1:19 and 1:1:20. In some embodiments, the silk fibroin fragment composition comprises silk fibroin fragments, a natural surfactant, and a thickening agent, wherein the weight ratio of the natural surfactant to the thickening/gelling agent to the silk fibroin fragments is a value selected from about 1:1:5 to about 1:1:11. In some embodiments, the silk fibroin fragment composition comprises silk fibroin fragments, a natural surfactant, and a thickening agent, wherein the weight ratio of the natural surfactant to the thickening/gelling agent to the silk fibroin fragments is about 1:5. In some embodiments, the silk fibroin fragment composition comprises silk fibroin fragments, a natural surfactant, and a thickening agent, wherein the weight ratio of the natural surfactant to the thickening/gelling agent to the silk fibroin fragments is about 1:11.

In some embodiments, the silk fibroin fragment composition comprises the natural surfactant in an amount ranging from about 0.2% w/w, w/v or v/v to about 1.0% w/w, w/v or v/v, the thickening/gelling agent in an amount ranging from about 0.2% w/w, w/v or v/v to about 1.0% w/w, w/v or v/v, and the silk fibroin protein fragment in an amount ranging from about 1.0% w/w, w/v or v/v to about 5.0% w/w, w/v or v/v by the basis of the silk fibroin fragment composition. In some embodiments, the silk fibroin fragment composition comprises the natural surfactant in an amount of about 1.0% w/w, w/v or v/v, the thickening/gelling agent in an amount of about 1.0% w/w, w/v or v/v, and silk fibroin protein fragment in an amount of about 5.0 w/w, w/v or v/v by the basis the silk fibroin fragment composition. In some embodiments, the silk fibroin fragment composition comprises the natural surfactant in an amount ranging from about 0.5% w/w, w/v or v/v to about 1.0% w/w, w/v or v/v, the thickening/gelling agent in an amount ranging from about 0.5 w/w, w/v or v/v to about 1.0% w/w, w/v or v/v, and the silk fibroin protein fragment in an amount ranging from about 5.0% w/w, w/v or v/v to about 5.5 w/w, w/v or v/v by the basis of the silk fibroin fragment composition. In some embodiments, the silk fibroin fragment composition comprises the natural surfactant in an amount of about 0.5% w/w, the thickening/gelling agent in an amount of about 0.5% w/w, and the silk fibroin protein fragment in an amount of about 5.5 w/w by the basis of the silk fibroin fragment composition.

In some embodiments, the natural surfactant is caprylyl/capryl glucoside and the thickening/gelling agent is xanthan gum. In some embodiments, the silk fibroin fragment composition comprises the caprylyl/capryl glucoside in an amount ranging from about 0.2% w/w, w/v or v/v to about 1.0% w/w, w/v or v/v, xanthan gum in an amount ranging from about 0.2% w/w, w/v or v/v to about 1.0% w/w, w/v or v/v, and the silk fibroin protein fragment in an amount ranging from about 1.0% w/w, w/v or v/v to about 5.0% w/w, w/v or v/v by the basis of the silk fibroin fragment composition. In some embodiments, the silk fibroin fragment composition comprises caprylyl/capryl glucoside in an amount of about 1.0% w/w, w/v or v/v, xanthan gum in an amount of about 1.0% w/w, w/v or v/v, and silk fibroin protein fragment in an amount of about 5.0 w/w, w/v or v/v by the basis the silk fibroin fragment composition. In some embodiments, the silk fibroin fragment composition comprises caprylyl/capryl glucoside in an amount ranging from about 0.5% w/w, w/v or v/v to about 1.0% w/w, w/v or v/v, xanthan gum in an amount ranging from about 0.5 w/w, w/v or v/v to about 1.0% w/w, w/v or v/v, and the silk fibroin protein fragment in an amount ranging from about 5.0% w/w, w/v or v/v to about 5.5 w/w, w/v or v/v by the basis of the silk fibroin fragment composition. In some embodiments, the silk fibroin fragment composition comprises caprylyl/capryl glucoside in an amount of about 0.5% w/w, xanthan gum in an amount of about 0.5% w/w, and the silk fibroin protein fragment in an amount of about 5.5% w/w by the basis of the silk fibroin fragment composition.

In some embodiments, the silk fibroin protein fragment composition comprises caprylyl/capryl glucoside, xanthan gum, and silk fibroin fragments in a weight ration at a value selected about 1:1:5 to about 1:1:11. In some embodiments, the silk fibroin protein fragment composition comprises caprylyl/capryl glucoside, xanthan gum, and silk fibroin fragments in a weight ratio of about 1:1:11.

In some embodiments, the natural surfactant is caprylyl/capryl glucoside and the thickening/gelling agent is carrageenan In some embodiments, the silk fibroin fragment composition comprises the caprylyl/capryl glucoside in an amount ranging from about 0.2% w/w, w/v or v/v to about 1.0% w/w, w/v or v/v, carrageenan in an amount ranging from about 0.2% w/w, w/v or v/v to about 1.0% w/w, w/v or v/v, and the silk fibroin protein fragment in an amount ranging from about 1.0% w/w, w/v or v/v to about 5.0% w/w, w/v or v/v by the basis of the silk fibroin fragment composition. In some embodiments, the silk fibroin fragment composition comprises caprylyl/capryl glucoside in an amount of about 1.0% w/w, w/v or v/v, carrageenan in an amount of about 1.0% w/w, w/v or v/v, and silk fibroin protein fragment in an amount of about 5.0 w/w, w/v or v/v by the basis the silk fibroin fragment composition. In some embodiments, the silk fibroin fragment composition comprises caprylyl/capryl glucoside in an amount ranging from about 0.5% w/w, w/v or v/v to about 1.0% w/w, w/v or v/v, carrageenan in an amount ranging from about 0.5 w/w, w/v or v/v to about 1.0% w/w, w/v or v/v, and the silk fibroin protein fragment in an amount ranging from about 5.0% w/w, w/v or v/v to about 5.5 w/w, w/v or v/v by the basis of the silk fibroin fragment composition. In some embodiments, the silk fibroin fragment composition comprises caprylyl/capryl glucoside in an amount of about 0.5% w/w, carrageenan in an amount of about 0.5% w/w, and the silk fibroin protein fragment in an amount of about 5.5% w/w by the basis of the silk fibroin fragment composition.

In some embodiments, the silk fibroin protein fragment composition comprises caprylyl/capryl glucoside, carrageenan, and silk fibroin fragments in a weight ration at a value selected about 1:1:5 to about 1:1:11. In some embodiments, the silk fibroin protein fragment composition comprises caprylyl/capryl glucoside, carrageenan, and silk fibroin fragments in a weight ratio of about 1:1:11.

In some embodiments, the silk fibroin fragment composition further comprises a buffering agent selected from the group consisting of phosphate buffered saline, borate buffered saline, citrate buffered saline, saline, sodium chloride, calcium chloride, magnesium chloride, potassium chloride, sodium bicarbonate, zinc chloride, hydrochloric acid, sodium hydroxide, edetate disodium, and combinations thereof.

In some embodiments, the silk fibroin fragment composition further comprises a density matching agent (also known as weighting agent) selected from the group consisting of ester gum (EG), damar gum (DG), sucrose acetate isobutyrate (SAIB), brominated vegetable oil (BVO), and combinations thereof. In some embodiments, the weighting agent concentrations required to match the oil and aqueous phase densities is of 25.0 wt. % for BVO, 55.0 wt. % for EG, 55.0 wt. % for DG, and 45.0 wt. % for SAIB.

In some embodiments, the silk fibroin fragment composition further comprises a preservative selected from the group consisting of sodium perborate, polyquaternium-1, benzalkonium chloride, brimonidine, brimonidine purite, polexitonium, and combinations thereof.

In some embodiments, the silk fibroin fragment composition has a hydrophilic-lipophilic balance (HLB) value of 0 to 19. In some embodiments, the silk emulsifier system has a HLB value selected from the group consisting from 0 to about 3, from about 3 to about 6, from about 6 to about 9, from about 9 to about 12, from about 12 to about 15, from about 15 to about 18, and greater than 18. In some embodiments, the silk fibroin fragment composition has a HLB value of about 1, about 2, about 3, about 4, about 5, about 6, about 7, about 8, about 9, about 10, about 11, about 12, about 13, about 14, about 15, about 16, about 17, or about 18. In some embodiments, the silk fibroin fragment composition has a HLB value ranging from about 6 to about 11. In some embodiments, the silk fibroin fragment composition has a HLB value ranging from about 12 to about 16.

In some embodiments, the silk fibroin fragment composition as described above is an oil based emulsion concentrate. The concentrate is homogeneous for at least 24 hours and emulsify readily on dilution into water.

In some embodiments, the silk fibroin fragment compositions as described above may be useful for manufacturing personal care products, feminine hygiene product, household products (e.g., dry and liquid laundry detergent, dish soap, dishwasher detergents, toilet bowl cleaner, upholstery cleaner, glass cleaner, general purpose cleaner, fabric softener), pet care product (e.g., shampoo), cosmeceutical products, dermatological products, nutraceutical products, food composition, beverage, eye drop formulation (e.g., artificial tears, ocular lubricants, lid scrubs), veterinary compositions, and pharmaceutical formulations.

In some embodiments, the silk fibroin fragment compositions have an aqueous phase containing a polyhydric alcohol and natural sugar surfactant as described above may be useful for manufacturing personal care products, household products, pet care product, cosmeceutical products, dermatological products, nutraceutical products, food composition, beverage, eye drop formulation (e.g., artificial tears, ocular lubricants, lid scrubs), veterinary compositions, and pharmaceutical formulations.

### 3. Silk Personal Care Composition

Human skin is made of proteins. Silk protein has high similarity to human skin. Most skin care products use silk fibroin protein raw material because this protein has a high percentage of glycine and alanine. The combination of glycine and alanine gives silk a remarkable effect on the skin. Coatings of silk protein on skin resistant removal, thereby providing a protective barrier against chemically- and biochemically induced skin damages. The silk personal care composition also provides a vehicle for administering an effective dose of personal care active agent to the skin surface.

Glycine and alanine are two of the simplest form of amino acids that the body is able to manufacture through the diet. Glycine produces a protein enriched with collagen. Glycine can help to repair skin damage and to speed up the wound healing process. Alanine is a great skin-conditioning agent. Most masks contain alanine as a leave-on ingredient and it can penetrate the epidermal cells. This helps to fill up lines and give skin a smoother appearance.

Due to silk fibroin is inherently stable to changes in temperature, pH and moisture and is mechanically robust. Silk fibroin protein is reputed to be an excellent water-binding and absorbing protein. Silk fibroin by nature has antibacterial and anti-fungal properties. Silk fibroin allows skin to breathe and is a natural moisture and heat regulator. Silk fibroin is naturally hypoallergenic and provides relief in conditions like eczema, sensitive skin, allergic rash, shingles, and psoriasis.

Skin care products incorporating silk fibroin protein can offer many benefits. Silk fibroin helps to calm inflamed skin by increasing cell metabolism and promoting blood circulation. In addition, the reduction of inflammation can help to promote even skin tone and ameliorate acne. The silk fibroin improves skin elasticity, reduce appearance of wrinkle and rejuvenating skin appearance. The silk fibroin increases blood circulation to scar tissue and reduces the appearance of scar. The silk fibroin protein imparts antioxidative effects and help to reverse the oxidative damage caused by free radicals and to repair/mitigate sun damage. When used in leave-on skincare products, silk fibroin imparts an attractive sheen and help the skin barrier to retain moisture.

In an embodiments, this disclosure provides compositions and methods for topical administration of skin treatment composition to the skin of mammals, specifically human, to protect skin by preserving and restoring the natural integrity of the skin.

In some embodiments, this disclosure provides personal care products in the form of an oil-in-water emulsion (o/w), or water-in-oil emulsion (w/o) stabilized with surfactant and/or co-surfactant. In some embodiments, the co-surfactant comprises protein, or peptide emulsifiers.

In one embodiment, the disclosure provides a silk personal care composition comprising SPF as defined herein, including, without limitation, silk fibroin protein and silk fibroin fragments, a polydispersity ranging from 1 to about 5; from 0 to 500 ppm lithium bromide; from 0 to 500 ppm sodium carbonate; and a carrier. In some embodiments, the silk fibroin fragments have an average weight average molecular weight selected from between about 1 kDa to about 5 kDa, from between about 5 kDa to about 10 kDa, from between about 6 kDa to about 17 kDa, from between about 10 kDa to about 15 kDa, from between about 15 kDa to about 20 kDa, from between about 17 kDa to about 39 kDa, from between about 20 kDa to about 25 kDa, from between about 25 kDa to about 30 kDa, from between about 30 kDa to about 35 kDa, from between about 35 kDa to about 40 kDa, from between about 39 kDa to about 80 kDa, from between about 40 kDa to about 45 kDa, from between about 45 kDa to about 50 kDa, from between about 60 kDa to about 100 kDa, and from between about 80 kDa to about 144 kDa.

In some embodiments, silk fibroin protein fragments useful for applications in personal care products also include silk fibroin protein derivatives such as low molecular weight silk fibroin peptides (weight average molecular weight selected from between about 5 kDa to about 38 kDa, about 14 kDa to about 30 kDa, or about 6 kDa to about 17 kDa). In some embodiments, silk fibroin peptides useful for applications in personal care products also include low molecular weight silk fibroin peptides having 2-50 amino acids. The low molecular weight silk fibroin peptides derived from silk fibroin protein hydrolysate can complement the natural moisturizing factors in the free amino acids to improve the skin moisture content. In some embodiments, the low molecular weight silk fibroin peptides can penetrate deep into the skin dermis to repair, replenish water, nourish skin, and improve the moisture balance.

In some embodiments, silk fibroin protein fragments useful for applications in personal care products also include lyophilized silk powder derived from the silk solution as described above.

In some embodiments, silk fibroin protein fragments useful for applications in personal care products also include silk fibroin protein derivatives such as silk fibroin protein amino acids derived from the hydrolyzed silk fibroin.

In some embodiments, the silk fibroin protein fragments as described herein can act as detergents for cleansing, wetting agents for better spreadability, emulsifiers to create stable mixtures of oil and water, film forming agent to form skin barrier layer, conditioning agents to improve the appearance of skin. In some embodiments, the silk solution exhibits enhanced emulsification power as compared with colloidal silk fibroin protein. In some embodiments, the silk personal care composition incorporated with silk fibroin protein fragment solution exhibits enhanced beneficial effects of the self-assembly and coating properties of the silk fibroin peptides in view of those of the full length silk fibroin protein with functional folding structure.

In some embodiments, the silk personal care composition further comprises one more personal care active agent to form various functional personal care products, wherein the personal active agent is selected from the group consisting of skin care active agent, cosmetically active agent, oral care active agent, deodorant and antiperspirant active agent, and nail care active agent.

In an embodiment, this disclosure provides a personal care composition comprising the silk fibroin protein fragments and the silk fibroin protein fragment based emulsion composition as described above, and a carrier.

In some embodiments, the silk personal care composition comprises silk fibroin protein derivatives containing (1) silk fibroin protein fragments having a weight average molecular weight selected from between about 5 kDa to about 144 kDa, (2) lyophilized silk powder derived from the silk solution, and (3) silk fibroin protein amino acids (glycine, alanine, serine) derived from the hydrolyzed silk fibroin and/or low molecular weight silk fibroin peptides having 2-50 amino acids.

In some embodiments, the silk fibroin fragments in the silk personal care composition have a polydispersity between 1 and about 1.5. In some embodiments, the silk fibroin fragments in the silk personal care composition have a polydispersity between about 1.5 and about 2.0. In some embodiments, the silk fibroin fragments in the silk personal care composition have a polydispersity between about 1.5 and about 3.0. In some embodiments, the silk fibroin fragments in the silk personal care composition have a polydispersity between about 2.0 and about 2.5. In some embodiments, the silk fibroin fragments in the silk personal care composition have a polydispersity between about 2.5 and about 3.0.

In some embodiments, the silk personal care composition comprises about 0.01 wt. % to about 10.0 wt. % of the silk fibroin fragments. In some embodiments, the silk personal care composition comprises about 0.01 wt. % to about 1.0 wt. % of the silk fibroin fragments. In some embodiments, the silk personal care composition comprises about 1.0 wt. % to about 2.0 wt. % of the silk fibroin fragments. In some embodiments, the silk personal care composition comprises about 2.0 wt. % to about 3.0 wt. % of the silk fibroin fragments. In some embodiments, the silk personal care composition comprises about 3.0 wt. % to about 4.0 wt. % of the silk fibroin fragments. In some embodiments, the silk personal care composition comprises about 4.0 wt. % to about 5.0 wt. % of the silk fibroin fragments. In some embodiments, the silk personal care composition comprises about 5.0 wt. % to about 6.0 wt. % of the silk fibroin fragments.

In some embodiments, the silk personal care composition further comprises about 0.01% (w/w) to about 10% (w/w) sericin by the total weight of the silk personal care composition. In some embodiments, the silk personal care composition further comprising about 0.01% (w/w) to about 10% (w/w) sericin by the total weight of the silk fibroin fragments.

In some embodiments, the silk fibroin fragments in the silk personal care composition do not spontaneously or gradually gelate and do not visibly change in color or turbidity when in an aqueous solution for at least 10 days prior to formulation into the silk personal care composition.

In some embodiments, the carrier comprises an oil phase. In some embodiments, the carrier comprises an aqueous phase. In some embodiments, the silk personal care composition further comprising an emulsifier other than silk fibroin protein fragments. In some embodiments, the carrier comprises an “oil-in-water” type emulsion or a “water-in-oil” type emulsion. In some embodiments, the carrier is obtained by diluting the emulsion concentrate of the silk fibroin fragment composition into water.

In some embodiments, the silk personal care composition forms an oral care composition. In some embodiments, the oral care composition further comprises an additive selected from the group consisting of a filler, a diluent, a remineralizing agent, an anti-calculus agent, an anti-plaque agent, a buffer, an abrasive, an alkali metal bicarbonate salt, a binder, a thickening agent, a humectant, a whitening agent, a bleaching agent, a stain removing agent, a surfactant, titanium dioxide, a flavoring agent, xylitol, a coloring agent, a foaming agent, a sweetener, an antibacterial agent, a preservative, a vitamin, a pH-adjusting agent, an anti-caries agent, a teeth whitening active agent, a desensitizing agent, a coolant, a salivating agent, a warming agent, a numbing agent, a chelating agent, and combinations thereof. In some embodiments, the oral care composition further comprises lyophilized silk powder derived from the silk solution described above. In some embodiments, the oral care composition is formulated as a product selected from the group consisting of a toothpaste, a dentifrice, a tooth powder, an oral gel, an aqueous gel, a non-aqueous gel, a mouth rinse, a mouth spray, a plaque removing liquid, a denture product, a dental solution, a lozenge, an oral tablet, a chewing gum, a candy, a fast-dissolving film, a strip, a dental floss, a tooth glossing product, a finishing product, and an impregnated dental implement.

In some embodiments, the oral care composition is formulated as a toothpaste comprising a tooth care active agent selected from the group consisting an abrasive, lyophilized silk powder, an anti-calculus agent, an anti-plaque agent, a humectant, a whitening agent, an anti-caries agent, a desensitizing agent, a coolant, a salivating agent, a warming agent, a numbing agent, and combinations thereof.

In some embodiments, the oral care composition is formulated as a tooth remineralization composition comprising a therapeutically effective amount of a remineralizing agent. In some embodiments, the remineralizing agent is selected from the group consisting of fluoride, calcium and/or phosphate, amorphous calcium phosphate (ACP), tricalcium phosphate, casein phosphoprotein-ACP, bioactive glass, calcium sodium phosphosilicate, arginine bicarbonate-calcium carbonate complex. In some embodiments, the tooth remineralization composition is formulated as a remineralizing gel, a remineralizing mouthwash, a remineralizing tooth powder, a remineralizing chewing gum, a remineralizing lozenge, or a remineralizing toothpaste.

In some embodiments, the silk personal care composition is a skin cleansing composition. In some embodiments, the emulsifier system for the skin cleansing composition is selected from the group consisting of a blend of silk fibroin protein fragments and an alkyl glucoside ester, or a blend of silk fibroin protein fragments and a sucrose ester. In some embodiments, the skin cleansing composition further comprises a dermatologically acceptable additive selected from the group consisting of a cleansing surfactant, a soap base, a detergent, a lathering surfactant, a skin conditioning agent, an oil control agent, an anti-acne agent, an astringent, a scrub particle or agent, an exfoliating particle or agent, a skin calming agent, a plant extract, an essential oil, a coolant, a humectant, a moisturizer, a structurant, a gelling agent, an antioxidant, an anti-aging compound, a sunscreen, a skin lightening agent, a sequestering agent, a preserving agent, a filler, a fragrance, a thickener, a wetting agent, a dye, a pigment, and combinations thereof. In some embodiments, the skin cleansing composition further comprises lyophilized silk powder derived from the silk solution described above. In some embodiments, the skin cleansing composition further comprises lyophilized silk powder derived from the silk solution and silk amino acids (glycine, alanine and serine) and/or silk peptides having 2-50 amino acids described above. In some embodiments, the skin cleansing composition is formulated as a product selected from the group consisting of a cleansing lotion, a cleansing milk, a cleansing gel, a cleansing soap bar, an exfoliating product, a bath and shower soap in bar, a body wash, a hand wash, a cleansing wipe, a cleansing pad, and a bath product.

In some embodiments, the silk personal care composition is a makeup composition. In some embodiments, the makeup composition further comprises a cosmetic ingredient selected from the group consisting of a skin conditioning agent, an oil control agent, an anti-acne agent, an astringent, a skin calming agent, a plant extract, an essential oil, a humectant, a moisturizer, a structurant, a gelling agent, an antioxidant, an anti-aging compound, a sunscreen, a skin lightening agent, a sequestering agent, a preserving agent, a filler, a fragrance, a thickener, a wetting agent, a dye, a pigment, a cosmetic powder, and combinations thereof. In some embodiments, the makeup composition further comprises lyophilized silk powder derived from the silk solution described above. In some embodiments, the makeup composition further comprises lyophilized silk powder derived from the silk solution and silk amino acids (glycine, alanine and serine) and/or silk peptides having 2-50 amino acids described above. In some embodiments, the makeup composition is formulated as a product selected from the group consisting of a color cosmetic, a mascara, a lipstick, a lip liner, an eye shadow, an eye-liner, a rouge, a face powder, a foundation, and a blush.

In some embodiments, the silk personal care composition is a cosmetic composition and the carrier is a cosmetically acceptable medium. In some embodiments, the cosmetic composition further comprises a cosmetic ingredient selected from the group consisting of a surfactant, a skin conditioning agent, an oil control agent, an anti-acne agent, an astringent, a scrub particle or agent, an exfoliating particle or agent, a skin calming agent, a plant extract, an essential oil, a coolant, a humectant, a moisturizer, a structurant, a gelling agent, an antioxidant, an anti-aging compound, a sunscreen, a skin lightening agent, a sequestering agent, a preserving agent, a filler, a fragrance, a thickener, a wetting agent, a dye, a pigment, a glitter, and combinations thereof. In some embodiments, the cosmetic composition further comprises lyophilized silk powder derived from the silk solution described above. In some embodiments, the cosmetic composition further comprises lyophilized silk powder derived from the silk solution and silk amino acids (glycine, alanine and serine) and/or silk peptides having 2-50 amino acids described above. In some embodiments, the cosmetic composition is formulated as a product selected from the group consisting of a cream, an emulsion, a shaving or after-shave cream, a foam, a conditioner, a cologne, a shaving or after-shave lotion, a perfume, a cosmetic oil, a facial mask, a moisturizer, an anti-wrinkle, an eye treatment, a tanning cream, a tanning lotion, a tanning emulsion, a sunscreen cream, a sunscreen lotion, a sunscreen emulsion, a tanning oil, a sunscreen oil, a hand lotion, and a body lotion.

In some embodiments, the silk personal care composition is a deodorant or antiperspirant composition and the carrier is a dermatologically acceptable medium. In some embodiments, the deodorant or antiperspirant composition further comprises an additive selected from the group consisting of a deodorant active, an antiperspirant active, an emollient, a humectant, a moisturizer, an astringent, an antiseptic agent, a gellant, a surfactant, a thickening agent, a cosmetic powder, a fragrance, a sunscreen, an antimicrobial, a preservative, a coloring agent, a filler, a co-emulsifier, a hardener, a strengthener, a chelating agent, a thixotropic agent, an oil absorbing agent, an antioxidant, and combinations thereof. In some embodiments, the deodorant or antiperspirant composition further comprises lyophilized silk powder derived from the silk solution described above. In some embodiments, the deodorant or antiperspirant composition further comprises lyophilized silk powder derived from the silk solution and silk amino acids (glycine, alanine and serine) and/or silk peptides having 2-50 amino acids described above. In some embodiments, the deodorant or antiperspirant composition is formulated as a product selected from the group consisting of a stick, a roll-on, a powder, a gel, an aerosol, a paste, and a cream. In some embodiments, the deodorant or antiperspirant composition has clear, transparent, or translucent appearance.

In some embodiments, the silk personal care composition is a nail care composition and the carrier is a dermatologically acceptable medium. In some embodiments, the nail care composition further comprises an additive selected from the group consisting of a film-forming agent, a suspending agent, a thixotropic agent, a coloring agent, a pigment, a glitter, a plasticizer, a thickening agent, a nail hydrating agent, a nail hardening agent, boric acid, a vitamin, a plant extract, an essential oil, a preservative, a mineral salt, a fruit extract, an algae extract, a fungus extract, a caviar extract, an aldehydes, a vegetable oil, an amino acid, a peptide, a protein, a ceramide, allantoin or an allantoin derivative, an organosilicon derivative, an antioxidant, a UV light absorber, a moisturizer, a stabilizer, a fragrance, a micronutrient, a dye, a pigment, and combinations thereof. In some embodiments, the nail care composition further comprises silk amino acids (glycine, alanine and serine) and/or silk peptides having 2-50 amino acids described above. In some embodiments, the nail care composition is formulated as a product selected from the group consisting of a nail varnish, a nail enamel, and a nail polish.

### 4. Carrier for Silk Personal Care Composition

### I. Cosmetically Acceptable Carriers

### (1) Emulsion Carrier

In some embodiments, the silk personal care composition comprises an emulsion as the cosmetically acceptable carrier. In some embodiments, the cosmetically acceptable carrier exists as a conventional emulsion. In some embodiments, the cosmetically acceptable carrier exits as a microemulsion. In some embodiments, the cosmetically acceptable carrier exits as a water-in-oil emulsion. In some embodiments, the cosmetically acceptable carrier exits as an oil-in-water emulsion. In some embodiments, the cosmetically acceptable carrier exits as a nano-emulsion. In some embodiments, the cosmetically acceptable carrier exits as a water-in-silicone oil emulsion. In some embodiments, the cosmetically acceptable carrier exits as a silicone oil-in-water emulsion. In some embodiments, the cosmetically acceptable carrier exits as O/W emulsion having multilamellar gel network. In some embodiments, the emulsion carrier comprises the synergistic emulsifier blend containing silk fibroin protein fragments and natural surfactant as described above, an oily component and water.

As used herein, the “conventional emulsions” have one continuous phase and one disperse phase, which is present as very small spheres stabilized by coating with surfactants. Depending on the nature of the continuous phase, the emulsions are described as oil-in-water or water-in-oil. These emulsions are kinetically stable in the ideal case, i.e. they are retained even for a prolonged period, but not indefinitely. During temperature fluctuations in particular, they may have a tendency toward phase separation because of sedimentation, creaming, thickening or flocculation.

As used herein, the “microemulsions” are thermodynamically stable, isotropic, fluid, optically clear single liquid phase containing a ternary system having three ingredients of an oily component, an aqueous component and a surfactant. Microemulsions arise when a surfactant, or more frequently a mixture of a surfactant and a co-surfactant, reduces the oil/water interfacial tension to extremely low values, often in the range 10⁻³ to 10⁻⁹ N/m (1 mN/m to 10⁻⁶ mN/m), preferably 10⁻⁴ to 10⁻⁶ N/m (0.1 mN/m to 0.001 mN/m), such that the two insoluble phases remain dispersed by themselves in a homogeneous manner as a result of the thermal agitation. Microemulsions often have bicontinuous structures with equilibrium regions, so-called subphases in the order of magnitude from 100 to 1000 Angstroms. The microemulsion refers to either one state of an O/W (oil-in-water) type microemulsion in which oil is solubilized by micelles, or a bicontinuous microemulsion in which the number of associations of surfactant molecules are rendered infinite so that both the aqueous phase and oil phase have a continuous structure.

For properties, the microemulsion appears transparent or translucent and may exist as a solution in a monophasic state in which all the formulated ingredients and components are uniformly dissolved therein.

Regardless of manufacturing processes, microemulsions may take the same state if they have the same formulation components and prepared at the same temperature. Therefore, the above-described three ingredients (oil, water and surfactant) and the remaining ingredients may be added and mixed in any orders as appropriate and may be agitated using mechanical forces at any power to consequently yield a microemulsion having substantially the same state (in appearance, viscosity, feeling of use, etc.).

Bicontinuous microemulsions comprise two phases, a water phase and an oil phase, in the form of extended adjoining and intertwined domains at whose interface stabilizing interface-active surfactants are concentrated in a monomolecular layer. Bicontinuous micro emulsions form very readily, usually spontaneously due to the very low interfacial tension, when the individual components, water, oil and a suitable emulsifier system, are mixed. Since the domains have only very small extensions in the order of magnitude of nanometers in at least one dimension, the microemulsions appear visually transparent and are thermodynamically, i.e. indefinitely, stable in a certain temperature range depending on the emulsifier system used.

As used herein, the term “nanoemulsions” refer to emulsions presenting transparent or translucent appearances due to their nano particle sizes, e.g. less than 1000 nm.

### A. Emulsifier System

Emulsifiers (e.g., surfactants) are substances that reduce the interfacial tension between liquid phases which are not miscible with one another, a polar phase, often water and a nonpolar, organic phase, and thus increase their mutual solubility. Surfactants have a characteristic structure feature of at least one hydrophilic and one hydrophobic structural unit. This structure feature is also referred to as amphiphilic. Emulsifier reduces the surface tension between the phases by being arranged at the interface between the two liquids. For stabilizing emulsions, mixture of emulsifiers are often used.

Anionic, cationic, amphoteric and nonionic surfactants have conventionally been used as emulsifiers for production of emulsified cosmetic materials by emulsification of water and oily substances. However, since synthetic surfactants have been implicated in the destruction of skin surface tissue and constituting a cause of liver damage when entering the body, numerous naturally derived protein-based emulsifiers including natural protein based emulsifiers have been employed because of their high safety.

Although emulsified cosmetic materials obtained using protein-based emulsifiers generally have a soft, moist feel during use, it is often the case finished products impart a crumbling feel and lack spreadability. The important factors for emulsifiers used in cosmetic products include not only safety and emulsifying power, but also feel during use. The disclosure provides the use of silk fibroin protein fragments as emulsifier (thereafter silk emulsifier) to stabilize the emulsion carrier for the personal care composition disclosed herein.

### (i) Additional Protein Emulsifier

Globular proteins play an important role in the formation and stabilization of oil-in-water emulsions. Globular protein emulsifiers can facilitate the production of small droplets to improve long-term stability of emulsions against droplet aggregation by lowering the interfacial tension during homogenization.

In some embodiments, in addition to the silk fibroin protein as emulsifier as described herein, an additional protein may be present in an amount of about 01. Wt. % to about 4.0 wt. % by the total weight of the silk personal care composition.

Proteins like casein are known for their emulsifying function, but if used alone, the obtained oil-in-water emulsion usually would not be heat-stable. Hence, although the emulsion might have high viscosity, it would not lead to products that have smooth texture and acceptable emulsion stability.

Additional protein emulsifier may be selected from the group consisting of hydrolyzed animal collagen obtained by enzymatic hydrolysis, hydrolyzed keratin, lexeine protein, egg white protein, egg yolk protein, lipoprotein, skim milk powder, casein, sodium caseinate, whey protein, hydrolyzed wheat protein, pea protein, soy protein, and mixture thereof.

### (ii) Natural Surfactant as Emulsifier

In some embodiments, this disclosure provides silk personal care composition comprises a natural surfactant as co-emulsifier selected from the group consisting of protein, peptide, sugar surfactant, biosurfactant, and combinations thereof. In some embodiments, the biosurfactant is selected from the group consisting of glycolipids, fatty acid, neutral lipid, phospholipids, polymeric biosurfactants, lipopeptides (surfactin, iturin, fengycin, lichenysin), and combinations thereof. In some embodiments, the glycolipid is selected from the group consisting of rhamnolipid, monorhamnolipid, dirhamnolipid, sophorolipid, lactonic sophorolipid, trehalolipid, mannosylerythritol lipid (ustilipid), and combinations thereof. In some embodiments, the sugar surfactant is selected from the group consisting of sucrose ester, sorbitan or sorbitol ester, alkyl polyglucoside, and combinations thereof. In some embodiments, the sugar surfactant is sucrose ester. In some embodiments, the sugar surfactant is alkyl polyglucoside.

As used herein, the term “natural surfactant” refers to surface-active substances derived from natural raw materials.

As used herein, the term “sugar surfactant” refers to sugar esters having carbohydrate (mono- or oligosaccharide) as hydrophilic head and fatty acid as hydrophobic tail. Sugar esters are non-ionic biodegradable surfactants available in a wide range of HLB values related to different sugar and fatty acid combinations.

As used herein, the term “biosurfactant” refers to natural amphiphilic compounds produced by yeast or bacteria. Biosurfactants are mainly classified according to their chemical structure and their microbial origin.

In some embodiments, the emulsion carrier for the silk personal care composition further comprise one or more sugar ester emulsifiers.

In some embodiments, the emulsifier system for the silk personal care composition comprises a synergistic emulsifier blend containing silk fibroin protein fragments and a sugar ester. In some embodiments, the emulsifier system for the silk personal care composition comprises a mixture of silk fibroin protein fragments as described above and a sucrose ester. In some embodiments, the emulsifier system for the silk personal care composition comprises a mixture of silk fibroin protein fragments as described above and an alkyl polyglucoside. The details on synergistic emulsifier blend are set forth above.
(iii) Synthetic Surfactants as Emulsifiers

In some embodiments, the emulsion carrier for the silk personal care composition may further comprise one or more ionic surfactants as co-emulsifiers.

An ionic surfactant is a surfactant that is ionized to have an electric charge in an aqueous solution; depending on the type of the electric charge, it is classified into ampholytic surfactants, cationic surfactants, or anionic surfactants. When an anionic surfactant and an ampholytic surfactant, or an anionic surfactant and a cationic surfactant, are mixed in an aqueous solution, the interfacial tension against oil decreases.

An ampholytic surfactant has at least one cationic functional group and one anionic functional group, is cationic when the solution is acidic and anionic when the solution is alkaline, and assumes characteristics similar to a nonionic surfactant around the isoelectric point.

Ampholytic surfactants are classified, based on the type of the anionic group, into the carboxylic acid type, the sulfuric ester type, the sulfonic acid type, and the phosphoric ester type. For the present disclosure, the carboxylic acid type, the sulfuric ester type, and the sulfonic acid type are preferable. The carboxylic acid type is further classified into the amino acid type and the betaine type. Particularly preferable is the betaine type.

Specific examples include: imidazoline type ampholytic surfactants (for example, 2-undecyl-1-hydroxyethyl-1-carboxymethyl-4,5-dihydro-2-imidazolium sodium salt and 1-[2-(carboxymethoxy)ethyl]-1-(carboxymethyl)-4,5-dihydro-2-norcocoalkylimidazolium hydroxide disodium salt); and betaine type surfactants (for example, 2-heptadecyl-N-carboxymethyl-N-hydroxyethyl imidazolinium betaine, lauryldimethylaminoacetic acid betaine, alkyl betaine, amide betaine, and sulfobetaine).

Examples of the cationic surfactant include quaternary ammonium salts such as cetyltrimethylammonium chloride, stearyltrimethylammonium chloride, benenyltrimethylammonium chloride, behenyldimethylhydroxyethylammonium chloride, stearyldimethylbenzylammonium chloride, and cetyltrimethylammonium methyl sulfate. Other examples include amide amine compounds such as stearic diethylaminoethylamide, stearic dimethylaminoethylamide, palmitic diethylaminoethylamide, palmitic dimethylaminoethylamide, myristic diethylaminoethylamide, myristic dimethylaminoethylamide, behenic diethylaminoethylamide, behenic dimethylaminoethylamide, stearic diethylaminopropylamide, stearic dimethylaminopropylamide, palmitic diethylaminopropylamide, palmitic dimethylaminopropylamide, myristic diethylaminopropylamide, myristic dimethylaminopropylamide, behenic diethylaminopropylamide, and behenic dimethylaminopropylamide.

In some embodiments, the emulsifier system for the silk personal care composition may further comprise one or more anionic surfactants. Anionic surfactants are classified into the carboxylate type such as fatty acid soaps, N-acyl glutamates, and alkyl ether acetates, the sulfonic acid type such as α-olefin sulfonates, alkane sulfonates, and alkylbenzene sulfonates, the sulfuric ester type such as higher alcohol sulfuric ester salts, and phosphoric ester salts. Preferable are the carboxylate type, the sulfonic acid type, and the sulfuric ester salt type; particularly preferable is the sulfuric ester salt type.

In some embodiments, the anionic surfactant for the personal care composition is selected from the group consisting of higher alkyl sulfuric acid ester salts (for example, sodium lauryl sulfate and potassium lauryl sulfate); alkyl ether sulfuric acid ester salts (e.g., POE-triethanolamine lauryl sulfate and sodium POE-lauryl sulfate); N-acyl sarcosinic acids (e.g., sodium lauroyl sarcosinate); higher fatty acid amide sulfonic acid salts (e.g., sodium N-myristoyl N-methyl taurate, Sodium N-cocoyl-N-methyl taurate, and Sodium jauroylmethyl taurate); phosphoric ester salts (e.g., sodium POE-oleyl ether phosphate and POE stearyl ether phosphoric acid); sulfosuccinates (e.g., sodium di-2-ethylhexylsulfosuccinate, sodium monolauroyl monoethanol amide polyoxyethylene sulfosuccinate, and sodium lauryl polypropylene glycol sulfosuccinate); alkyl benzene sulfonates (e.g., sodium linear dodecyl benzene sulfonate, triethanolamine linear dodecyl benzene sulfonate, and linear dodecyl benzene sulfonic acid); higher fatty acid ester sulfates (e.g., hydrogenated coconut oil aliphatic acid glyceryl sodium sulfate); N-acyl glutamates (e.g., mono sodium N-lauroylglutamate, disodium N-stearoylglutamate, and sodium N-myristoyl-L-glutamate); sulfated oils (e.g., turkey red oil); POE-alkyl ether carboxylic acid; POE-alkyl aryl ether carboxylate; α-olefin sulfonate; higher fatty acid ester sulfonates; sec-alcohol sulfates; higher fatty acid alkyl amide sulfates; sodium lauroyl monoethanolamine succinates; ditriethanolamine N-palmitoylaspartate; and sodium caseinate.

In some embodiments, the emulsifier system for the silk personal care composition may further comprise one or more nonionic surfactants as co-emulsifiers. The nonionic surfactant preferably has an HLB value of 8.9-14. It is generally known that the solubility into water and the solubility into oil balance when the HLB is 7. That is, a surfactant preferable for the present disclosure would have medium solubility in oil/water.

The nonionic surfactants may include: (1) polyethylene oxide extended sorbitan monoalkylates (e.g., polysorbates); (2) polyalkoxylated alkanols; (3) polyalkoxylated alkylphenols include polyethoxylated octyl or nonyl phenols having HLB values of at least about 14, which are commercially available under the trade designations ICONOL® and TRITON®; (4) polaxamers. Surfactants based on block copolymers of ethylene oxide (EO) and propylene oxide (PO) may also be effective. Both EO-PO-EO blocks and PO-EO-PO blocks are expected to work well as long as the HLB is at least about 14, and preferably at least about 16. Such surfactants are commercially available under the trade designations PLURONIC® and TETRONIC® from BASF; (5) polyalkoxylated esters: polyalkoxylated glycols such as ethylene glycol, propylene glycol, glycerol, and the like may be partially or completely esterified, i.e. one or more alcohols may be esterified, with a (C8 to C22) alkyl carboxylic acid. Such polyethoxylated esters having an HLB of at least about 14, and preferably at least about 16, may be suitable for use in compositions of the present disclosure; (6) alkyl polyglucosides. This includes glucopon 425, which has a (C8 to C16) alkyl chain length; (7) sucrose fatty acid ester having high HLB value (8-18): sucrose cocoate, sucrose dilaurate, sucrose distearate, sucrose hexaerucate, sucrose hexaoleate/hexapalmitate/hexstearate, sucrose hexapalmitate, sucrose laurate, sucrose myristate, sucrose oleate, sucrose palmitate, sucrose pentaerucate, sucrose polybehenate, sucrose polycottonseedate, sucrose polylaurate, sucrose polylinoleate, sucrose polyoleate, sucrose polypalmate, sucrose polysoyate, sucrose polystearate, sucrose ricinoleate, sucrose stearate, sucrose tetraisostearate, sucrose trilaurate.

In some embodiments, the emulsifier system comprises a lipophilic nonionic surfactants selected from the group consisting of sorbitan fatty acid esters (e.g., sorbitan mono oleate monooleate, sorbitan mono isostearate monoisostearate, sorbitan mono laurate monolaurate, sorbitan mono palmitate monopalmitate, sorbitan mono stearate monostearate, sorbitan sesquioleate, sorbitan trioleate, diglyceryl sorbitan penta-2-ethylhexylate, diglyceryl sorbitan tetra-2-ethylhexylate); glyceryl and polyglyceryl aliphatic acids (e.g., mono cottonseed oil fatty acid glycerine, glyceryl monoerucate, glyceryl sesquioleate, glyceryl monostearate, α,α′-glyceryl oleate pyroglutamate, monostearate glyceryl malic acid); propylene glycol fatty acid esters (e.g., propylene glycol monostearate); hydrogenated castor oil derivatives; glyceryl alkylethers, and combination thereof.

In some embodiments, the emulsifier system comprises a hydrophilic nonionic surfactants selected from the group consisting of POE-sorbitan fatty acid esters (e.g., POE-sorbitan monooleate, POE-sorbitan monostearate, POE-sorbitan monooleate, and POE-sorbitan tetraoleate); POE sorbitol fatty acid esters (e.g., POE sorbitol monolaurate, POE-sorbitol monooleate, POE-sorbitolpentaoleate, and POE-sorbitol monostearate); POE-glyceryl fatty acid esters (e.g., POE-monooleates such as POE-glyceryl monostearate, POE-glyceryl monoisostearate, and POE glycerin glyceryl triisostearate); POE-fatty acid esters (e.g., POE-distearate, POE-monodioleate, and ethylene glycol distearate); POE-alkylethers (e.g., POE-lauryl ether, POE-oleyl ether, POE-stearyl ether, POE-behenyl ether, POE 2-octyl dodecyl ether, and POE-cholesterol ether); pluronics (e.g., Pluronic F-68); POE-POP-alkylethers (e.g., POE-POP-cetyl ether, POE-POP₂-decyl tetradecyl ether, POE-POP-monobutyl ether, POE-POP-lanolin hydrate, and POE-POP glycerin glyceryl ether); tetra POE-tetra POP-ethylenediamino condensates (e.g., tetronic); POE-castor oil hydrogenated castor oil derivatives (e.g., POE-castor oil, POE-hydrogenated castor oil, POE-hydrogenated castor oil monoisostearate, POE-hydrogenated castor oil triisostearate, POE-hydrogenated castor oil monopyroglutamic monoisostearic diester, and POE-hydrogenated castor oil maleic acid); POE-beeswax-lanolin derivatives (e.g., POE-sorbitol beeswax); alkanol amides (e.g., palm oil fatty acid diethanol amide, laurate monoethanolamide, and fatty acid isopropanol amide); POE-propylene glycol fatty acid esters; POE-alkylamines; POE-fatty acid amides; sucrose fatty acid esters; alkyl ethoxydimethylamine oxides; and trioleyl phosphoric acid.

In some embodiments, the emulsifier system comprises mono-glycerol derivatives and/or diglycerol derivatives. Specific examples include: monoglycerol derivatives such as monoglycerol monooctanoate, monooctyl monoglyceryl ether, monoglycerol monononanoate, monononyl monoglyceryl ether, monoglycerol monodecanoate, monodecyl monoglyceryl ether, monoglycerol monoundecylenate, monoundecylenyl glyceryl ether, monoglycerol monododecanoate, monododecyl monoglyceryl ether, monoglycerol monotetradecanoate, monoglycerol monohexadecanoate, monoglycerol monooleate, and monoglycerol monoisostearate, as well as diglycerol derivatives such as diglycerol monooctanoate, monooctyl diglyceryl ether, diglycerol monononanoate, monononyl diglyceryl ether, diglycerol monodecanoate, monodecyl diglyceryl ether, diglycerol monoundecylenate, monoundecylenyl glyceryl ether, diglycerol monododecanoate, monododecyl diglyceryl ether, diglycerol monotetradecanoate, diglycerol monohexadecanoate, diglycerol monooleate, and diglycerol monoisostearate.

In some embodiments, the emulsifier system is incorporated in the emulsion carrier at a weight percent ranging from 0.1 wt. % to 5.0 wt. % by the total weight of the personal care composition. In some embodiments, the emulsifier system is incorporated in the emulsion carrier at a weight percent ranging from 0.1 wt. % to 3.0 wt. % by the total weight of the personal care composition. In some embodiments, the emulsifier system is incorporated in the emulsion carrier at a weight percent ranging from 0.1 wt. % to 2.0 wt. % by the total weight of the personal care composition.

In some embodiments, the emulsion containing silk fibroin protein fragment is substantially free of synthetic emulsifier.

### B. Oil Phase

In some embodiments, the emulsion carrier comprises an oil phase emulsified with the emulsifier system containing the silk emulsifier as described above. The fatty materials may be useful for forming the oil phase. The fatty material is selected from the group consisting of hydrocarbon oils, silicon oil, higher fatty acids, higher alcohols, synthetic ester oils, liquid oils/fats, solid oils/fats, waxes, emu oil, and combination thereof.

In some embodiments, the emulsion carrier comprises a synergistic emulsifier blend containing silk fibroin protein fragments and one or motr sugar surfactant as co-emulsifier and an oil selected from the group consisting of mineral oil, hydrogenated cotton seed oil, linseed oil, mustard oil, neem oil, niger seed oil, oiticica oil, olive oil, palm oil, palm kernel oil, peanut oil, perilla oil, poppy seed oil, rape seed oil, safflower oil, sesame oil, soybean oil, eucalyptus oil, lavender oil, tea tree oil, green tea oil, rosemary oil, patchouli oil, cedar wood atlas oil, clover leaf oil, palmarosa oil, grapefruit oil, bergamot calabrian oil, pine oil, cardamom oil, peppermint oil, cinnamon leaf oil, and ylang oil, vitamin A, vitamin E, vitamin K, and combinations thereof.

In some embodiments, the emulsion carrier comprises emu oil as oily component. Emu oil, an animal-derived lipid composition, is extracted from the Emu. Emu oil is comprised of approximately 50% to 70% monounsaturated fatty acids, with the rest being both saturated and polyunsaturated fatty acids. Emu oil contains triglyceride esters of long chain fatty acids including oleic acid and linoleic acid as well as the saturated fatty acids, palmitic acid and stearic acid (neutral lipid). Emu oil is non-comedogenic, has anti-inflammatory properties, is deeply moisturizing, and deeply penetrating the skin epidermis. The ability of emu oil to penetrate the stratum corneum dermal barrier and concomitantly act as a carrier makes it highly valuable for use in cosmetic composition for the treatment of a variety of skin conditions.

Emu oil is useful to treat pigmentation disorders such as hypopigmentation, stimulate the proliferation of cells in mammalian skin tissue, and stimulating melanogenesis to enhance skin tanning, useful for treating aging, photo-damaged skin and skin ulcerations, dry skin (lack of dermal hydration), undue skin slackness (i.e., insufficient skin firmness) and insufficient sebum secretion.

Emu oil is commercially available from New World Technology, Inc., Greenwich, Conn., under the name “Kalaya Oil™”. In some embodiments, the emu oil is presented in the cosmetically acceptable carrier in an amount ranging from about 1.0 wt. % to about 99 wt. % by the total weight of the cosmetically acceptable carrier. In some embodiments, the emu oil is presented in the cosmetically acceptable carrier in an amount selected from the group consisting of about 1.0 wt. %, about 5.0 wt. %, about 10.0 wt. %, about 15.0 wt. %, about 20.0 wt. %, about 25.0 wt. %, about 30.0 wt. %, about 35.0 wt. %, about 40.0 wt. %, about 45.0 wt. %, about 50.0 wt. %, about 55.0 wt. %, about 60.0 wt. %, about 65.0 wt. %, about 70.0 wt. %, about 75.0 wt. %, about 80.0 wt. %, about 85.0 wt. %, about 90.0 wt. %, about 95.0 wt. %, and about 99.0 wt. %.

In an embodiment, the oil phase optionally comprises a wax. The wax is selected from the group consisting of polyethylene wax, polypropylene wax, beeswax, candelilla wax, paraffin wax, ozokerite, microcrystalline waxes, carnauba wax, cotton wax, esparto wax, carnauba wax, bayberry wax, tree wax, whale wax, montan wax, bran wax, lanolin, kapok wax, lanolin acetate, liquid lanolin, sugar cane wax, lanolin fatty acid isopropyl ester, hexyl laurate, reduced lanolin, jojoba wax, hard lanolin, shellac wax, POE lanolin alcohol ether, POE lanolin alcohol acetate, POE cholesterol ether, lanolin fatty acid polyethylene glycol, POE hydrogenated lanolin alcohol ether, and combination thereof.

In an embodiment, the oil phase optionally comprises an ester oil. The ester oil is selected from the group consisting of cholesteryl isostearate, isopropyl palmitate, isopropyl myristate, neopentylglycol dicaprate, isopropyl isostearate, octadecyl myristate, cetyl 2-ethylhexanoate, cetearyl isononanoate, cetearyl octanoate, isononyl isononanoate, isotridecyl isononanoate, glyceryl tri-2-ethylhexanoate, glyceryl tri(caprylatelcaprate), diethylene glycol monoethyl ether oleate, dicaprylyl ether, caprylic acid/capric acid propylene glycol diester, and combination thereof.

In an embodiment, the oil phase optionally comprises a glyceride fatty ester. As used herein, the term “glyceride fatty esters” refers to the mono-, di-, and tri-esters formed between glycerol and long chain carboxylic acids such as C₆-C₃₀ carboxylic acids. The carboxylic acids may be saturated or unsaturated or contain hydrophilic groups such as hydroxyl. Preferred glyceride fatty esters are derived from carboxylic acids of carbon chain length ranging from C₁₀ to C₂₄, preferably C₁₀ to C₂₂ most preferably C₁₂ to C₂₀.

In an embodiment, the oil phase optionally comprises synthetic ester oils. In some embodiments, the synthetic ester oil is selected from the group consisting of isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexylate, dipentaerythritol fatty acid ester, N-alkyl glycol monoisostearate, neopentyl glycol dicaprate, diisostearyl malate, glyceryl di-2-heptylundecanoate, trimethylolpropane tri-2-ethylhexylate, trimethylolpropane triisostearate, pentaneerythritol tetra-2-ethylhexylate, glyceryl tri-2-ethylhexylate, trimethylolpropane triisostearate, cetyl 2-ethylhexanoate, 2-ethylhexyl palmitate, glyceryl trimyristate, tri-2-heptylundecanoic glyceride, castor oil fatty acid methyl ester, oleyl oleate, cetostearyl alcohol, acetoglyceride, 2-heptylundecyl palmitate, diisopropyl adipate, N-lauroyl-L-glutamic acid-2-octyldodecyl ester, di-2-heptylundecyl adipate, ethyl laurate, di-2-ethylhexyl cebatate. 2-hexyldecyl myristate, 2-hexyldecyl palmitate, 2-hexyldecyl adipate, diisopropyl cebatate, 2-ethylhexyl succinate, ethyl acetate, butyl acetate, amyl acetate and triethyl |citrate, and combination thereof.

In an embodiment, the oil phase optionally comprises ether oil. In some embodiments, the ether oils are selected from the group consisting of alkyl-1,3-dimethylethyl ether, nonylphenyl ether, and combination thereof.

In an embodiment, the oil phase optionally comprises higher fatty acids. As used herein, the higher fatty acids have a carbon number ranging from 8 to 22. In some embodiments, the higher fatty acid is selected from the group consisting of lauric acid, myristic acid, palmitic acid, stearic acid, behenic acid, oleic acid, 12-hydroxystearic acid, undecylenic acid, tall oil, isostearic acid, linoleic acid, linolenic acid, eicosapentaenoic acid (EPA), docosahexaenoic acid (DHA), and combination thereof.

In an embodiment, the oil phase optionally comprises higher fatty alcohols. As used herein, the higher fatty alcohols have a carbon number ranging from 8 to 22. In some embodiments, the higher fatty acid is selected from the group consisting of straight chain alcohols (for example, lauryl alcohol, cetyl alcohol, stearyl alcohol, behenyl alcohol, myristyl alcohol, oleyl alcohol, and cetostearyl alcohol) and branched chain ethyl alcohols (for example, mono stearyl glyceryl ether (batyl alcohol), 2-decyltetradecynol, lanolin alcohol, cholesterol, phytosterol, hexyl dodecanol, isostearyl alcohol, and octyl dodecanol), and combination thereof.

In an embodiment, the oil phase optionally comprises one or more silicone oils. As used herein, the term “silicone oil” (also as silicone fluid) is used herein to designate water-insoluble silicone polymers that are applied to skin to improve its feel or appearance. Silicone oils can provide the skin with a silky, lubricious feel. They can also provide a lusterization effect. These results are obtained by coating skin with thin films of silicone oil. Since silicone oils are substantially water-insoluble, after application to the skin they tend to remain thereon despite rinsing with water.

In some embodiments, the oil phase comprises a non-volatile silicone, which may be a polyalkyl siloxane, a polyalkylaryl siloxane, or mixtures thereof. Suitable polyalkyl siloxanes include polydimethyl siloxanes having a viscosity of from 5 to 100,000 centistokes at 25° C. These siloxanes are available commercially from the General Electric Company as the VISCASIL® series and from Dow Corning as the DC 200 series.

In some embodiments, the silicone oil is selected from the group consisting of linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol. The polymethylphenyl polysiloxanes having a viscosity of from 15 to 65 centistokes at 25° C. These siloxanes are available commercially from Dow Corning as DC-556 grade silicone fluid.

In some embodiments, the silicone oils is selected from the group consisting of methyl polysiloxane, decamethylcydopentasiloxane, octamethylcydotetrasiloxane, and combination thereof.

In some embodiments, the silicone oil comprises volatile silicon oil selected from the group consisting of cyclic siloxanes have four to eight membered rings. In some embodiments, the volatile silicone comprises cyclomethicone selected from the group consisting of dodecamethyl cyclohexasiloxane, decamethylcydopentasiloxane (D5), octamethylcydotetrasiloxane (D4), and combination thereof.

In some embodiments, the oil phase comprises liquid oils/fats. In some embodiments, the liquid oils/fats are selected from the group consisting of avocado oil, tsubaki oil, turtle oil, macademia nut oil, corn oil, mink oil, olive oil, rape seed oil, egg yolk oil, sesame seed oil, persic oil, wheat germ oil, sasanqua oil, castor oil, linseed oil, safflower oil, cotton seed oil, perilla oil, soybean oil, peanut oil, tea seed oil, kaya oil, rice bran oil, Chinese wood oil, Japanese wood oil, jojoba oil, germ oil, triglycerol, glyceryl trioctanoate and glyceryl triisopalmitate, and combination thereof.

In some embodiments, the oil phase comprises solid fats/oils. In some embodiments, the solid oils/fats are selected from the group consisting of cacao butter, coconut oil, horse tallow, hardened coconut oil, palm oil, beef tallow, sheep tallow, hardened beef tallow, palm kernel oil, pork tallow, beef bone tallow, Japanese core wax, hardened oil, neatsfoot tallow, Japanese wax and hydrogenated castor oil, and combination thereof.

In some embodiments, the oil phase comprises vegetable oils. In some embodiments, the vegetable oils are selected from the group consisting of buriti oil, soybean oil, olive oil, tea tree oil, rosemary oil, jojoba oil, coconut oil, sesame seed oil, sesame oil, palm oil, avocado oil, babassu oil, rice oil, almond oil, argon oil, sunflower oil, safflower oil, black currant seed, borage oil, palm kernel oil, and combination thereof. In some embodiments, the vegetable oil is selected from the group consisting of coconut oil, sunflower oil and sesame oil. In some embodiments, the oily component is selected from olive oil, cocoa butter, palm stearin, sunflower oil, soybean oil and coconut oil.

In some embodiments, the oil phase for the silk personal care composition comprises lipid material. In some embodiments, the lipid materials are selected from the group consisting of soybean oil, ceramides, phospholipids (e.g., soy lecithin, egg lecithin), egg phosphatides, soybean phosphatides, phosphatides of marine origin, glycolipids, medium chain triglyceride (MCT), olive oil, sesame oil, sunflower oil, flax seed oil, cotton seed oil, egg-yolk, fish oil, krill oil, and combination thereof.

In some embodiments, the oil phase for the silk personal care composition comprises hydrocarbon oil. As used herein, the hydrocarbon oils have average carbon chain length less than 20 carbon atoms. Suitable hydrocarbon oils include cyclic hydrocarbons, straight chain aliphatic hydrocarbons (saturated or unsaturated), and branched chain aliphatic hydrocarbons (saturated or unsaturated). Straight chain hydrocarbon oils will typically contain from about 6 to about 16 carbon atoms, preferably from about 8 up to about 14 carbon atoms. Branched chain hydrocarbon oils can and typically may contain higher numbers of carbon atoms, e.g. from about 6 up to about 20 carbon atoms, preferably from about 8 up to about 18 carbon atoms. Suitable hydrocarbon oils of the disclosure will generally have a viscosity at ambient temperature (25 to 30° C.) of from 0.0001 to 0.5 Pa·s, preferably from 0.001 to 0.05 Pa·s, more preferably from 0.001 to 0.02 Pa·s.

In some embodiments, the hydrogen carbon oils are selected from the group consisting of liquid petrolatum, squalane, pristane, paraffin, isoparaffin, ceresin, squalane, squalene, mineral oil, light mineral oil, blend of light mineral oil and heavy mineral oil, polyisobutene, hydrogenated polyisobutene, terpene oil and combination thereof.

In some embodiments, the hydrogen carbon oils light mineral oil. As used herein, mineral oils are clear oily liquids obtained from petroleum oil, from which waxes have been removed, and the more volatile fractions removed by distillation. The fraction distilling between 250° C. to 300° C. is termed mineral oil, and it consists of a mixture of hydrocarbons, in which the number of carbon atoms per hydrocarbon molecule generally ranges from C10 to C40. Mineral oil may be characterized in terms of its viscosity, where light mineral oil is relatively less viscous than heavy mineral oil, and these terms are defined more specifically in the U.S. Pharmacopoeia, 22nd revision, p. 899 (1990). A commercially available example of a suitable light mineral oil for use in the disclosure is Sirius® M40 (carbon chain length C0-C28 mainly C12-C20, viscosity 4.3×10 Pa·s), available from Silkolene®. Other hydrocarbon oils that may be used in the disclosure include relatively lower molecular weight hydrocarbons including linear saturated hydrocarbons such a tetradecane, hexadecane, and octadecane, cyclic hydrocarbons such as dioctylcyclohexane (e.g. CETIOL® S from Henkel), branched chain hydrocarbons (e.g. ISOPAR® and ISOPAR® V from Exxon Corp.).

In some embodiments, the fatty material for the oil phase is selected from the group consisting of neopentyl glycol diheptanoate, propylene glycol dicaprylate, dioctyl adipate, coco-caprylate/caprate, diethylhexyl adipate, diisopropyl dimer dilinoleate, diisostearyl dimer dilinoleate, butyrospermum parkii (shea butter), C12-C13 alkyl lactate, di-C12-C13 alkyl tartrate, tri-C12-C13 alkyl citrate, C12-C15 alkyl lactate, ppg dioctanoate, diethylene glycol dioctanoate, meadow foam oil, C12-15 alkyl oleate, tridecyl neopentanoate, cetearyl alcohol and polysorbate 60, C18-C26 triglycerides, cetearyl alcohol & cetearyl glucoside, acetylated lanolin, vp/eicosene copolymer, glyceryl hydroxystearate, C18-36 acid glycol ester, C18-36 triglycerides, glyceryl hydroxystearate, and mixtures thereof. In some embodiments, the fatty material for the oil phase is selected from the group consisting of cetyl alcohol & glyceryl stearate & PEG-75, stearate & ceteth-20 & steareth-20, lauryl glucoside & polyglyceryl-2 dipolyhydroxystearate, beheneth-25, polyamide-3 & pentaerythrityl tetra-di-t-butyl hydroxycinnamate, polyamide-4 and PEG-100 stearate, potassium cethylphosphate, stearic acid, and hectorites.

In some embodiments, the fatty material for the oil phase is selected from the group consisting of paraffin oil, glyceryl stearate, isopropyl myristate, diisopropyl adipate, cetylstearyl 2-ethylhexanoate, hydrogenated polyisobutene, vaseline, caprylic/capric triglycerides, microcrystalline wax, lanolin and stearic acid, silicone oils and combination thereof.

In an embodiment, the fatty material for the oil phase is selected from the group consisting of jojoba oil, olive oil, camellia oil, avocado oil, cacao oil, sunflower oil, persic oil, palm oil, castor oil, buriti oil, medium chain triglycerides, and combinations thereof.

In an embodiment, the emulsion carrier comprises one or more sucrose ester as co-emulsifier and the oily materials emulsifiable by the silk emulsifier is selected from the group consisting of a vegetable oil, isododecane, and isohexadecane, and one or more oily esters of fatty acids, wherein the vegetable oil is selected from jojoba oils and/or camellia oils, wherein the oily esters are selected from isononyl isononanoate and coco caprylate.

In some embodiments, the oil phase is present in the cosmetically acceptable carrier at a weight percent ranging from 1.0 wt. % to about 95 wt. % by the total weight of the cosmetically acceptable carrier. In some embodiments, the oil phase is present in the cosmetically acceptable carrier at a weight percent ranging from 45.0 wt. % to about 95 wt. % by the total weight of cosmetically acceptable carrier. In some embodiments, the oil phase is present in the cosmetically acceptable carrier at a weight percent ranging from 45.0 wt. % to about 65.0 wt. % by the total weight of the cosmetically acceptable carrier. In some embodiments, the oil phase is present in the cosmetically acceptable carrier at a weight percent ranging from 5.0 wt. % to about 45 wt. % by the total weight of the cosmetically acceptable carrier. In some embodiments, the oil phase is present in the cosmetically acceptable carrier at a weight percent ranging from 5.0 wt. % to about 35 wt. % by the total weight of the cosmetically acceptable carrier. In some embodiments, the oil phase is present in the cosmetically acceptable carrier at a weight percent ranging from 10.0 wt. % to about 25 wt. % by the total weight of the cosmetically acceptable carrier.

In some embodiments, the oil phase is presented in the cosmetically acceptable carrier in a weight percent ranging from about 50.0 wt. % to 95.0 weight % by the total weight of the cosmetically acceptable carrier. In some embodiments, the oil phase is presented in the cosmetically acceptable carrier in a weight percent ranging from about 5 wt. % to 45 weight % by the total weight of the cosmetically acceptable carrier, because such a content allows the emulsion carrier to have a stability over a wider temperature range around the room temperatures and a good feeling.

### C. Aqueous Phase

In some embodiments, the aqueous phase for the emulsion carrier comprises water, an aqueous solution, a blend of alcohol and water, or a lyotropic liquid crystalline phase as aqueous carrier. Selection of the water contained in the silk personal care composition of the present disclosure is not limited in particular; specific examples include purified water, ion-exchanged water, and tap water. In some embodiments, the aqueous further comprise one or more small molecule polyhydric alcohols selected from the group consisting of ethanediol, propanediol, glycerol, butanediol, butantetraol, xylitol, sorbitol, inositol, ethylene glycol, polyethylene glycol. In some embodiments, the aqueous phase further comprise one or more low alcohol solvent including methanol, ethanol, and isopropanol.

The blend ratio of water and polyhydric alcohol is determined appropriately based on emulsion formulation types.

In some embodiments, the emulsion comprises from about 50.0 wt. % to about 98.0 wt. % of the aqueous phase by the total weight of the cosmetically acceptable carrier. In some embodiments, the emulsion comprises from about 60.0 wt. % to about 90.0 wt. % of the aqueous phase by the total weight of the cosmetically acceptable carrier. In some embodiments, the amount of the aqueous phase in the emulsion carrier is selected from the group consisting of about 50.0 wt. %, about 51.0 wt. %, about 52.0 wt. %, about 53.0 wt. %, about 54.0 wt. %, about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, about 80.0 wt. %, about 81.0 wt. %, about 82.0 wt. %, about 83.0 wt. %, about 84.0 wt. %, about 85.0 wt. %, about 86.0 wt. %, about 87.0 wt. %, about 88.0 wt. %, about 89.0 wt. %, about 90.0 wt. %, about 91.0 wt. %, about 92.0 wt. %, about 93.0 wt. %, about 94.0 wt. %, about 95.0 wt. %, about 96.0 wt. %, about 97.0 wt. %, and about 98.0 wt. %, by the total weight of the cosmetically acceptable carrier.

In some embodiments, the synergistic emulsifier blend is present in the aqueous phase. In some embodiments, the synergistic emulsifier blend is present in the oil phase

### (2). Multi-Lamellar Liquid Crystal Gel Network (Structured Fluid, Gel Network).

The stratum corneum serves important barrier functions, specifically to prevent excessive trans-epidermal water loss and protect against ingress of foreign chemicals and microorganism. Emulsifiers that form multi-lamellar liquid crystals are marketed as mimicking the multi-lamellar lipid structure of the stratum corneum. Because they are biomimetic, lamellar liquid crystals serve as barrier and water-retention functions. The multi-lamellar liquid crystal networks can be formed in oil-in-water emulsions by combining a high HLB primary emulsifier (e.g., hydrophilic surfactant) and a second low-to-medium HLB co-emulsifier (e.g., a hydrophobic surfactant). The high HLB primary emulsifier reduces interfacial tension and facilitates the formation of small oil droplets in the outer aqueous phase. The low HLB co-emulsifier forms a gel network. This network structure stabilizes the emulsion by preventing creaming and coalescence of the oil droplets as well as by building viscosity.

In some embodiments, this disclosure provides a cosmetically acceptable oil-in-water emulsion carrier comprising a sugar surfactant having HLB value ranging from about 10 to about 16 (thereafter high HLB surfactant) and silk fibroin protein fragments disclosed herein, and at least one solid fatty alcohol that forms a multi-lamellar liquid crystalline network to effectively moisturize and protect the skin and to provide a useful vehicle for delivery cosmetically active agents. In some embodiments, the high HLB surfactant comprises a mixture of sucrose palmitate and sucrose laurate in 3:1 to 1:3 weight ratio. In some embodiments, the high HLB surfactant comprises a glucoside surfactant.

The calculated HLB for silk fibroin protein fragments as described herein is 6.2. The high HLB surfactants that are known to produce lamellar liquid crystals is selected from the group consisting of sucrose ester of fatty acids, sucrose monostearate, sucrose distearate, blend of sucrose cocoate and sorbitan stearate, blend of cetearyl alcohol and cetearyl glucoside (Montanov 68™).

The alkyl polyglucoside surfactant emulsifies all types of oils (ester oil, mineral oil, vegetable oil, and silicone oil). The emulsions stabilized by alkyl polyglucoside gives rich feel, produces cream to butter textures, promotes liquid crystals around oil droplets and in the continuous phase, and provides long lasting moisturizing effect 5 hours after application.

In some embodiments, the high HLB surfactant is a mixture of two surfactants selected from the group consisting of sucrose stearate, sucrose palmitate, sucrose cocoate, and sucrose laurate. In some embodiments, the high HLB surfactant comprises a mixture of sucrose palmitate and sucrose laurate in 1:1 weight ratio. In some embodiments, the high HLB surfactant is present in the emulsion at an amount ranging from about 0.2 wt. % to about 3.0 wt. % by the total weight of the emulsion. In some embodiments, the high HLB surfactant is present in the emulsion at an amount ranging from about 0.2 wt. % to about 0.3 wt. % by the total weight of the gelled emulsion carrier to form light lotion. In some embodiments, the high HLB surfactant is present in the emulsion at an amount ranging from about 0.4 wt. % to about 0.6 wt. % by the total weight of the gelled emulsion carrier to form a heavy lotion. In some embodiments, the high HLB surfactant is present in the emulsion at an amount ranging from about 0.5 wt. % to about 0.75 wt. % by the total weight of the gelled emulsion carrier to form a soft cream. In some embodiments, the high HLB surfactant is present in the emulsion at an amount ranging from about 0.8 wt. % to about 1.2 wt. % by the total weight of the gelled emulsion carrier to form a firm cream.

In some embodiments, the high HLB surfactant is present in the emulsion at an amount ranging from about 0.8 wt. % to about 1.2 wt. % by the total weight of the gelled emulsion carrier.

In some embodiments, the silk fibroin protein fragments is presented in the gel network at an amount ranging from about 0.1 wt. % to 3.0 wt. % by the total weight of the gelled emulsion carrier.

The aqueous phase of the gelled emulsion is present in an amount ranging from about 65 wt. % to about 95 wt. % by the total weight of the emulsion. The aqueous phase may contain water or a mixture of water and polyhydric alcohol. In some embodiments, the aqueous phase contains water and glycerin. In some embodiments, glycerin is present in an amount ranging from about 5 wt. % to about 20 wt. % by the total weight of the emulsion.

In some embodiments, the solid fatty alcohol is selected from the group consisting of cetyl alcohol, stearyl alcohol, behenyl alcohol, and combinations thereof. In some embodiments, the solid fatty alcohol is a mixture of cetyl alcohol and stearyl alcohol having a weight ratio of 30:70 to 70:30. In some embodiments, the solid fatty alcohol is a mixture of cetyl alcohol and stearyl alcohol in 1:1 weight ratio.

The silk fibroin protein fragments based multi-lamellar liquid crystalline network allows preparing uniform dispersions of creams and lotions, both with high gloss, good surface spreading and water resistance. The oils suitable for the gelled emulsion is selected from the group consisting of hydrocarbon oil, mineral oil, petrolatum, polydecene, polyolephin, glyceride, silicone oil, lanolin, lecithin, sunflower oil, rapeseed oil, soy bean oil, algae oil, and synthetic fatty ester oil.

In some embodiments, the multi-lamellar liquid crystalline gel network of the emulsion further comprise a thickener selected from the group consisting of acrylic acid polymer, carrageenan, xanthan gum, guar gum, and magnesium aluminum silicate, and combinations thereof. In some embodiments, the thickener is carrageenan, xanthan gum and guar gum. In some embodiments, the thickener is presented in the emulsion at an amount ranging from about 0.05 wt. % to about 0.5 wt. % by the total weight of the emulsion.

### (3). Aerosol Foam Carrier

In some embodiments, this disclosure provides an aerosol foam carrier for the personal care composition comprising the emulsion carrier as described above and a propellant that serves to expel the other materials from the container. Aerosol foams are obtaining by dispensing an emulsion charged with propellants from a pressurized container such that the pressurized emulsion and propellant expands to forming the foam bubbles (e.g., mousses).

The aerosol propellant included in silk personal care compositions of the present disclosure can be any liquefiable gas conventionally used for aerosol containers. Examples of suitable propellants include dimethyl ether and hydrocarbon propellants such as propane, n-butane and iso-butane. The propellants may be used singly or admixed. Water insoluble propellants, especially hydrocarbons, are preferred because they form emulsion droplets on agitation and can create suitable mousse foam densities when needed.

The amount of the propellant used is governed by factors well known in the aerosol art. For mousses, the level of propellant is generally up to 35.0 wt. %, preferably from 2.0 wt. % to 30.0 wt. %, most preferably from 3.0 wt. % to 15.0 wt. % by weight based on total weight of the composition. In some embodiments, the propellant is selected from the group consisting of propane, n-butane, isobutene, dimethyl ether, and combinations thereof. Preferably, the propellant comprises dimethyl ether and at least one of propane, n-butane and isobutene. The method of aerosol foam compositions as described herein follows the conventional aerosol filling procedures. The composition ingredients (not including the propellant) are charged into a suitable pressurizable container that is sealed and then charged with the propellant according to conventional techniques.

In some embodiments, this disclosure provides foam compositions comprising an oil-in-water emulsion having an emulsified oil phase by the synergistic emulsifier blends as described above.

The aerosol foam products can be easily distributed on the skin and leave good skin feeling. The physical structure of the foam acts positively on the protective function of the skin. Upon application, balanced foam formulations have stable multidispersed structures that form on the skin a network structures to develop a long lasting protective action due to high affinity to the skin and excellent film forming properties from the silk fibroin protein fragments.

### II. Orally Acceptable Carrier

“Orally acceptable carrier” refers to any safe and effective materials for use in the compositions of the present disclosure. Such materials include fluoride ion sources, additional anticalculus agents, buffers, abrasive polishing materials, peroxide sources, alkali metal bicarbonate salts, thickening materials, humectants, water, surfactants, titanium dioxide, flavor system, sweetening agents, xylitol, coloring agents, and mixtures thereof.

The oral care acceptable carrier is a toothpaste, dentifrice, tooth powder, topical oral gel, mouth rinse, denture product, mouth spray, lozenge, oral tablet, chewing gum, fast-dissolving films, strips, or impregnated dental implement. In some embodiments, the orally acceptable carrier comprises one or more compatible solid or liquid filler diluents or encapsulating substances that are suitable for topical oral administration.

The choice of orally acceptable carrier to be used is determined by the way the composition is to be introduced into the oral cavity. If a toothpaste, including tooth gels, other dentifrices, etc. is to be used, then a toothpaste carrier is chosen (e.g., abrasive materials, foaming agents, binders, humectants, flavoring and sweetening agents).

If a mouth rinse is to be used, then a mouth rinse carrier is chosen. Similarly, if a mouth spray is to be used, then a mouth spray carrier is chosen or if a lozenge is to be used, then a lozenge carrier is chosen (e.g., a candy base). If a chewing gum is to be used, then a chewing gum carrier is chosen. If a sachet is to be used, then a sachet carrier is chosen, sachet bag, flavoring, and sweetening agents. If a subgingival gel is to be used, for delivery of actives into the periodontal pockets or around the periodontal pockets, then a subgingival gel carrier is chosen.

### III. Aqueous Liquid Carrier Substantially Free of Non-Silk Surfactant

In some embodiments, the silk personal care product comprises an aqueous liquid carrier substantially free of non-silk surfactant. As used herein, the term “substantially free of non-silk surfactant” refers to the amount of non-silk surfactant in the aqueous liquid carrier at an amount less than 1.0 wt. %. In some embodiments, the amount of non-silk surfactant in the aqueous liquid carrier at an amount less than a weight percent selected from the group consisting of 1.0 wt. %, 0.9 wt. %, 0.8 wt. %, 0.7 wt. %, 0.6 wt. %, 0.5 wt. %, 0.4 wt. %, 0.3 wt. %, 0.2 wt. %. 0.1 wt. %, 0.05 wt. %, 0.01 wt. %, and 0.001 wt. %. In some embodiments, the amount of non-silk surfactant in the aqueous liquid carrier is 0%.

In some embodiments, the aqueous liquid carrier is selected from water, an aqueous solution, an alcohol, a blend of alcohol and water, or a lyotropic liquid crystalline phase. Water is an ingredient that constitutes the water phase of the emulsion carrier for the silk personal care composition. Selection of the water contained in the silk personal care composition of the present disclosure is not limited in particular; specific examples include purified water, ion-exchanged water, and tap water.

In some embodiments, the aqueous liquid carrier comprises one or more small molecule polyhydric alcohols selected from the group consisting of ethanediol, propanediol, glycerol, butanediol, butantetraol, xylitol, sorbitol, inositol, ethylene glycol, polyethylene glycol. In some embodiments, the aqueous liquid carrier comprises water and glycerol. In some embodiments, the aqueous liquid carrier comprises water and glycerol in a weight ratio of water to glycerol at 1:10. In some embodiments, the aqueous liquid carrier comprises water and glycerol in a weight ratio of water to glycerol selected from 1:10, 1:9, 1:8, 1:7, 1:6, 1:5, 1:4, 1:3, 1:2, and 1:1. In some embodiments, the aqueous liquid carrier comprises water and glycerol in a weight ratio of water to glycerol at 1:1. In some embodiments, the aqueous liquid carrier comprises water and glycerol in a weight ratio of water to glycerol at 1:10. In some embodiments, the aqueous liquid carrier comprises silk fibroin protein fragments and glycerol in a weight ratio of silk fibroin protein fragments to glycerol selected from 1:10, 1:9, 1:8, 1:7, 1:6, 1:5, 1:4, 1:3, 1:2, and 1:1. In some embodiments, the aqueous liquid carrier comprises silk fibroin protein fragments and glycerol in a weight ratio of silk fibroin protein fragments to glycerol at 1:1.

In some embodiments, the pH of the aqueous liquid phase is adjusted ranging from about 4.0 to about 9.0. In some embodiments, the pH of the aqueous liquid phase is adjusted ranging from about 4.5 to about 8.5. In some embodiments, the pH of the aqueous liquid phase is adjusted ranging from about 5.0 to about 7.0. The pH-adjusting agent may include buffer (e.g. PBS buffer), alkali metal salt, acid, citric acid, succinic acid, phosphoric acid, sodium hydroxide, ammonium hydroxide, ethanolamine, sodium carbonate, and combination thereof.

In some embodiments, the silk personal care composition comprises from about 1.0 wt. % to about 99.0 wt. % of the aqueous liquid carrier. In some embodiments, the silk personal care composition comprises from about 5.0 wt. % to about 45.0 wt. % of the aqueous liquid carrier. In some embodiments, the silk personal care composition comprises from about 5.0 wt. % to about 35.0 wt. % of the aqueous liquid carrier. In some embodiments, the silk personal care composition comprises from about 10.0 wt. % to about 30.0 wt. % of the aqueous liquid carrier. In some embodiments, the silk personal care composition comprises from about 45.0 wt. % to about 95.0 wt. % of the aqueous liquid carrier. In some embodiments, the silk personal care composition comprises from about 60.0 wt. % to about 90.0 wt. % of the aqueous liquid carrier. In some embodiments, the silk personal care composition comprises from about 45.0 wt. % to about 75.0 wt. % of the aqueous liquid carrier. In some embodiments, the silk personal care composition comprises from about 60.0 wt. % to about 75.0 wt. % of the aqueous liquid carrier. In some embodiments, the amount of the aqueous liquid carrier in the silk personal care composition is selected from the group consisting of about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, about 11.0 wt. %, about 12.0 wt. %, about 13.0 wt. %, about 14.0 wt. %, about 15.0 wt. %, about 16.0 wt. %, about 17.0 wt. %, about 18.0 wt. %, about 19.0 wt. %, about 20.0 wt. %, about 21.0 wt. %, about 22.0 wt. %, about 23.0 wt. %, about 24.0 wt. %, about 25.0 wt. %, about 26.0 wt. %, about 27.0 wt. %, about 28.0 wt. %, about 29.0 wt. %, about 30.0 wt. %, about 31.0 wt. %, about 32.0 wt. %, about 33.0 wt. %, about 34.0 wt. %, about 35.0 wt. %, about 36.0 wt. %, about 37.0 wt. %, about 38.0 wt. %, about 39.0 wt. %, about 40.0 wt. %, about 41.0 wt. %, about 42.0 wt. %, about 43.0 wt. %, about 44.0 wt. %, about 45.0 wt. %, about 46.0 wt. %, about 47.0 wt. %, about 48.0 wt. %, about 49.0 wt. %, about 50.0 wt. %, about 51.0 wt. %, about 52.0 wt. %, about 53.0 wt. %, about 54.0 wt. %, about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, about 80.0 wt. %, about 81.0 wt. %, about 82.0 wt. %, about 83.0 wt. %, about 84.0 wt. %, about 85.0 wt. %, about 86.0 wt. %, about 87.0 wt. %, about 88.0 wt. %, about 89.0 wt. %, about 90.0 wt. %, about 91.0 wt. %, about 92.0 wt. %, about 93.0 wt. %, about 94.0 wt. %, about 95.0 wt. %, about 96.0 wt. %, about 97.0 wt. %, and about 98.0 wt. % by the total weight of the silk personal care composition.

### IV. Non-Aqueous Liquid Carrier

In some embodiments, the silk personal care composition comprises a non-aqueous liquid carrier. Non-aqueous liquid carrier as used herein means that the liquid carrier is substantially free of water. In the present disclosure, “the liquid carrier being substantially free of water” means that: the liquid carrier is free of water; or, if the liquid carrier contains water, the level of water is very low. In the present disclosure, the level of water, if included, 1% or less, preferably 0.5% or less, more preferably 0.3% or less, still more preferably 0.1% or less, even more preferably 0% by weight of the silk personal care composition.

In some embodiments, the non-aqueous liquid carrier comprises an oily material selected from the group consisting of mineral oil, hydrocarbon oils, hydrogenated polydecene, polyisobutene, isoparaffin, isododecane, isohexadecane, volatile silicone oil, non-volatile silicone oil, isohexadecane, squalene, squalene, ester oil and combination thereof. In some embodiments, the non-aqueous liquid carrier comprises an oily material selected from the group consisting of white mineral oils, squalane, hydrogenated polyisobutene, isohexadecane, and isododecane. In some embodiments, the non-aqueous liquid carrier comprises squalane and hydrogenated polyisobutene. In some embodiments, the non-aqueous liquid carrier comprises white mineral oils, isohexadecane, and isododecane.

In some embodiments, the non-aqueous liquid carrier comprises a volatile isoparaffin having from about 8 to about 20 carbon atoms. In some embodiments, the non-aqueous liquid carrier comprises a volatile isoparaffin having from about 8 to about 16 carbon atoms. In some embodiments, the non-aqueous liquid carrier comprises a volatile isoparaffin having from about 10 to about 16 carbon atoms. In some embodiments, the volatile isoparaffin is selected from the group consisting of trimer, tetramer, and pentamer of isobutene, and mixtures thereof. Commercially available isoparaffin hydrocarbons may have distributions of its polymerization degree, and may be mixtures of, for example, trimer, tetramer, and pentamer. What is meant by tetramer herein is that a commercially available isoparaffin hydrocarbons in which tetramer has the highest content, i.e., tetramer is included at a level of preferably 70% or more, more preferably 80% or more, still more preferably 85% or more.

In some embodiments, the volatile isoparaffin is a mixture of several grades of isoparaffins. In some embodiments, the volatile isoparaffin has a viscosity range selected from: about 0.5 mm²·s⁻¹ to about 50 mm²·s⁻¹, about 0.8 mm²·s⁻¹ to about 40 mm²·s⁻¹, about 1 mm²·s⁻¹ to about 30 mm²·s⁻¹, about 1 mm²·s⁻¹ to about 20 mm²·s⁻¹, and about 1 mm²·s⁻¹ to about 10 mm²·s⁻¹, at 37.8° C. When using two or more isoparaffin hydrocarbon solvents, it is preferred that the mixture of isoparaffin hydrocarbon solvents have the above viscosity.

In some embodiments, the non-aqueous liquid carrier comprises ester oil. In some embodiments, the ester oils have an HLB of 3 or less, and as liquid at room temperature. In some embodiments, the ester oil is selected from the group consisting of methyl palmitate, methyl stearate, methyl oleate, methyl linoleate, and methyl laurate. In an embodiment, the ester oil methyl stearate.

In some embodiments, the ester oil is included in the non-aqueous liquid carrier at a weight percent selected from: about 0.1 wt. % to about 25 wt. %, about 0.5 wt. % to about 15 wt. %, about 1.0 wt. % to about 10 wt. %, about 1.0 wt. % to about 5.0 wt. % by the total weight of the silk personal care composition, in view of the balance between conditioned feel and product stability, and/or in view of prevent foaming.

In some embodiments, the non-aqueous liquid carrier comprises fatty esters selected from the group consisting of trimethyloylpropane tricaprylate/tricaprylate, C12-C15 alkyl benzoate, ethylhexyl stearate, ethylhexyl cocoate, decyl oleate, decyl cocoate, ethyl oleate, isopropyl myristate, ethylhexyl perlagonate, pentaerythrityl tetracaprylate/tetracaprate, PPG-3 benzyl ether myristate, propylene glycol dicaprylate/dicaprate, ethylhexyl isostearate, ethylhexyl palmitate and natural oils such as Glycine soja, Helianthus annuus, Simmondsia chinensis, Carthamus tinctorius, Oenothera biennis, Rapae oleum, and combination thereof.

In some embodiments, the non-aqueous liquid carrier comprises glyceride fatty ester. In some embodiments, the suitable glyceride fatty esters for use in skin oils of the disclosure have a viscosity at ambient temperature (25 to 30° C.) of from 0.01 to 0.8 Pa·s, preferably from 0.015 to 0.6 Pa·s, more preferably from 0.02 to 0.065 Pa·s.

In an embodiment, the fatty material comprises a glyceride fatty ester. As used herein, the term “glyceride fatty esters” refers to the mono-, di-, and tri-esters formed between glycerol and long chain carboxylic acids such as C6-C30 carboxylic acids. The carboxylic acids may be saturated or unsaturated or contain hydrophilic groups such as hydroxyl. Preferred glyceride fatty esters are derived from carboxylic acids of carbon chain length ranging from C10 to C24, preferably C10 to C22, most preferably C12 to C 20, most preferably C12 to C 18. In some embodiments, glyceride fatty ester is a medium-chain triglyceride having C6-C12 fatty acid chain.

In some embodiments, glyceride fatty ester is sourced from varieties of vegetable and animal fats and oils, such as camellia oil, coconut oil, castor oil, safflower oil, sunflower oil, peanut oil, cottonseed oil, corn oil, olive oil, cod liver oil, almond oil, avocado oil, palm oil, sesame oil, lanolin, and soybean oil. Synthetic oils include trimyristin, triolein and tristearin glyceryl dilaurate. Vegetable derived glyceride fatty esters include almond oil, castor oil, coconut oil, palm kernel oil, sesame oil, sunflower oil and soybean oil.

In some embodiments, the glyceride fatty ester is selected from coconut oil, sunflower oil, almond oil and mixtures thereof.

The non-aqueous liquid carrier is included at a level by weight of the silk personal care composition of, from about 50.0 wt. % to about 99.9 wt. %, from about 60.0 wt. % to about 99.8 wt. %, more preferably from about 65.0 wt. % to about 98.0 wt. % by the total weight of the silk personal care composition.

### 5. Silk Personal Care Products

### I. Skin Cleansing Product Carrier

Personal cleansing and conditioning products have traditionally been marketed in a variety of forms such as bar soaps, creams, lotions, and gels. These formulations have attempted to satisfy a number of criteria to be acceptable to consumers. These criteria include cleansing effectiveness, skin feel, mildness to skin, good lather volume, cleanse the skin or hair gently, cause little or no irritation, and not leave the skin or hair overly dry after frequent use. However, these traditional forms of personal cleansing products have the inherent problem of balancing—cleansing efficacy against delivering skin conditioning benefits. In a typical cleansing composition, the conditioning ingredients are difficult to formulate because many conditioners are incompatible with the surfactants, resulting in an undesirable non-homogenous mixture. Many conditioning agents have the disadvantage of suppressing lather generation. Lather suppression is also problematic because many consumers seek cleansing products that provide a rich, creamy, and generous lather. In addition, it is difficult to deposit of water-soluble conditioning agents with traditional cleansers since water-soluble conditioning agents are rinsed away.

Thus, there is a need for novel skin cleansing products that provides effective cleansing and yet deposit both oil soluble conditioning agents (e.g., emollients and lipids) and water soluble conditioning agents (e.g., humectants) to the skin.

In one embodiment, the disclosure provides a silk personal care composition comprising SPF as defined herein, including, without limitation, silk fibroin protein and silk fibroin fragments. In some embodiments, the silk personal care composition further comprises a natural surfactant. In some embodiments, the silk personal care composition further comprises a thickening/gelling agent. In some embodiments, the silk personal care composition comprises a silk fibroin protein fragment composition of the disclosure.

In some embodiments, the silk personal care composition is a skin cleansing composition. In some embodiments, the skin cleansing composition further comprises a dermatologically acceptable additive selected from the group consisting of a cleansing surfactant, a soap base, a detergent, a lathering surfactant, a skin conditioning agent, an oil control agent, an anti-acne agent, an astringent, a scrub particle or agent, an exfoliating particle or agent, a skin calming agent, a plant extract, an essential oil, a coolant, a humectant, a moisturizer, a structurant, a gelling agent, an antioxidant, an anti-aging compound, a sunscreen, a skin lightening agent, a sequestering agent, a preserving agent, a filler, a fragrance, a thickener, a wetting agent, a dye, a pigment, and combinations thereof. In some embodiments, the skin cleansing composition is formulated as a product selected from the group consisting of cleansing water, a cleansing lotion, a cleansing milk, a cleansing gel, a cleansing soap bar, an exfoliating product, a bath and shower soap in bar, a body wash, a hand wash, a cleansing wipe, a cleansing pad, and a bath product.

The silk skin cleansing compositions described herein are most useful for cleaning of the face and removing decorative cosmetics. The cleansing composition containing silk fibroin protein fragments has the advantage to have high affinity to skin for imparting both cleansing and delivery of long lasting skin conditioning benefits due to the silk fibroin film coated on the skin surface as compared to the conventional soap cleansing products.

### (1) Cleansing Phase

In some embodiments, the cleansing phase comprises a cleansing surfactant system and the emulsion carrier described above. In some embodiments, the cleansing phase comprises a cleansing surfactant system and the silk fibroin protein fragment compositions as described above.

### A. Cleansing Surfactant System

In some embodiments, the skin cleansing composition comprises a cleansing surfactant system to provide cleansing performance. The cleansing surfactant system may comprise surfactant selected from anionic detersive surfactant, zwitterion or amphoteric detersive surfactant, or a combination thereof. Such surfactants should be physically and chemically compatible with the essential components described herein, or should not otherwise unduly impair product stability, aesthetics or performance.

In some embodiments, the cleansing surfactant system in the skin cleansing composition comprises a lathering surfactant selected from the group consisting of anionic lathering surfactants, nonionic lather surfactants, amphoteric lathering surfactants, and mixtures thereof. The term “lathering surfactant” as used herein refers to a surfactant, which when combined with water and mechanically agitated, generates a foam or lather. Preferably, these surfactants or combinations of surfactants should be mild, which means that these surfactants provide sufficient cleansing or detersive benefits but do not overly dry the skin, and yet produce rich lathering.

In some embodiments, the cleansing phase comprises silk fibroin protein fragments described above as a lathering surfactant. The natural silk fibroin protein and peptides derived thereof provide skin cleansing and conditioning benefits including skin moisturizing, skin barrier protection by coating the skin surface with a thin film of silk fibroin protein fragments.

In some embodiments, the cleansing phase comprises about 2.0 wt. % to about 5.0 wt. % of silk fibroin-based protein fragments that are substantially devoid of sericin, wherein the silk fibroin-based protein fragments have a weight average molecular weight selected from between about 5 kDa to about 80 kDa, wherein the silk fibroin-based protein fragments have a polydispersity of between about 1.5 and about 3.0. In some embodiments, the cleansing phase comprises about 2.0 wt. % to about 5.0 wt. % of any silk fibroin-based protein fragments described herein.

In some embodiments, the cleansing phase comprises about 2.0 wt. % to about 5.0 wt. % of silk fibroin-based protein fragments that are substantially devoid of sericin, wherein the silk fibroin-based protein fragments have a weight average molecular selected from between about 5 kDa to about 17 kDa, wherein the silk fibroin-based protein fragments have a polydispersity of between about 1.5 and about 3.0.

In some embodiments, the cleansing phase comprises about 2.0 wt. % to about 5.0 wt. % of silk fibroin-based protein fragments that are substantially devoid of sericin, wherein the silk fibroin-based protein fragments have a weight average molecular weight selected from between about 5 kDa to about 25 kDa, wherein the silk fibroin-based protein fragments have a polydispersity of between about 1.5 and about 3.0.

In some embodiments, the cleansing phase comprises about 2.0 wt. % to about 5.0 wt. % of silk fibroin-based protein fragments that are substantially devoid of sericin, wherein the silk fibroin-based protein fragments have a weight average molecular weight selected from between about 17 kDa to about 39 kDa, wherein the silk fibroin-based protein fragments have a polydispersity of between about 1.5 and about 3.0.

In some embodiments, the cleansing phase comprises about 2.0 wt. % to about 5.0 wt. % of silk fibroin-based protein fragments that are substantially devoid of sericin, wherein the silk fibroin-based protein fragments have a weight average molecular selected from between about 25 kDa to about 40 kDa, wherein the silk fibroin-based protein fragments have a polydispersity of between about 1.5 and about 3.0.

In some embodiments, the cleansing phase comprises about 2.0 wt. % to about 5.0 wt. % of silk fibroin-based protein fragments that are substantially devoid of sericin, wherein the silk fibroin-based protein fragments have a weight average molecular weight selected from between about 40 kDa to about 65 kDa, wherein the silk fibroin-based protein fragments have a polydispersity of between about 1.5 and about 3.0.

In some embodiments, the cleansing phase comprises about 2.0 wt. % to about 5.0 wt. % of silk fibroin-based protein fragments that are substantially devoid of sericin, wherein the silk fibroin-based protein fragments have a weight average molecular weight selected from between about 39 kDa to about 80 kDa, wherein the silk fibroin-based protein fragments have a polydispersity of between about 1.5 and about 3.0.

In some embodiments, the cleansing surfactant system optionally comprise one or more additional surfactant selected from soap, anionic surfactant, and amphiprotic surfactant.

In some embodiments, the cleansing surfactant system optionally comprises a fatty acid soap as cleansing agent. Soap as used herein refers to the salts of fatty acids of which the fatty acid has an alkyl carbon chain of 12 to 32 carbon atoms. In some embodiments, the cleansing phase comprises C12-C14 fatty acid soap, C16-C18 fatty acid soap, or 80/20 blend of 80% C16-C18/20% C12-C14 fatty acid soap. The C16-C18 fatty acid soap can be obtained from tallow, and the C12-C14 fatty acid soap can be obtained from lauric, palm kernel, or coconut oils. In some embodiments, the fatty acid soaps are selected from the group consisting of sodium laurate and sodium palmitate. In some embodiments, small amount of fatty acid (e.g., 1.0 wt. %) is added to the fatty acid soap to improve lather quality.

In some embodiments, the cleansing surfactant system optionally comprises sulfonates and sulfates as cleansing agent. The suitable sulfonates and sulfates are selected from the group consisting of alkyl sulfates, alkylether sulfates, alkyl sulfonates, alkylether sulfonates, alkylbenzene sulfonates, alkylbenzene ether sulfates, alkylsulfoacetates, secondary alkane sulfonates, secondary alkylsulfates, alkyl sulfosuccinates, and combination thereof. The alkyl and acyl groups generally contain from 8 to 18, preferably from 10 to 16 carbon atoms and may be unsaturated. The alkyl ether sulphates, alkyl ether sulphosuccinates and salts thereof may contain from 1 to 20 ethylene oxide or propylene oxide units per molecule.

In some embodiments, the anionic detersive surfactant is selected from the group consisting of sodium oleyl succinate, ammonium lauryl sulphosuccinate, sodium lauryl sulphate, sodium lauryl ether sulphate, sodium lauryl ether sulphosuccinate, ammonium lauryl sulphate, ammonium lauryl ether sulphate, sodium dodecylbenzene sulphonate, triethanolamine dodecylbenzene sulphonate, sodium cocoyl isethionate, sodium lauryl isethionate, lauryl ether carboxylic acid, sodium lauryl sulphate and sodium lauryl ether sulphate (EO)₁₋₃, sodium lauryl ether sulphate (EO)₁₋₃, sodium lauryl ether sulphate (EO), and sodium N-lauryl sarcosinate.

In some embodiments, the optional surfactant for the cleansing phase may include water-soluble salts of higher fatty acid monosulfated monoglyceride, for example, sodium salt of the monosulfated monoglyceride of hydrogenated coconut oil fatty acids, sodium lauryl sulfate, sodium dodecyl benzene sulfonate, alkyl sulfoacetates, and fatty acid esters of 1,2-dihydroxy propane sulfonate.

In some embodiments, the cleansing surfactant system optionally comprises phosphates and phosphonates as detersive surfactant. The suitable phosphates and phosphonates are selected from the group consisting of alkyl phosphates, alkylether phosphates, aralkylphosphates, aralkylether phosphates, trilaureth-4-phosphate (a mixture of mono-, di- and tri-(alkyltetraglycolether)-o-phosphoric acid esters, HOSTAPHAT® 340KL from Clariant Corp.), and PPG-5 ceteth 10 phosphate (CRODAPHOS® SG from Croda Inc., Parsipanny, N.J.).

In some embodiments, the cleansing surfactant system optionally comprises amine oxides as cleansing agent. The suitable amine oxide surfactants are selected from the group consisting of lauryldimethylamine oxide, laurylamidopropyldimethylamine oxide, cetyl amine oxide, and combinations thereof.

In some embodiments, the cleansing surfactant system optionally comprises an anionic detergents selected from the group consisting of ammonium laurel sulfosuccinate, ammonium laurel sulfate, triethanolamine dodecalbenzene sulfonate, ammonium laureth sulfate, and combinations thereof. In some embodiments, the cleansing surfactant system optionally comprises laurel sulfates.

In some embodiments, small amounts of free fatty acid, e.g. about 0.01 wt. % to about 1.0 wt. % is added to the cleansing phase to produce creamier and thicker lather. In order to provide a combination of quick lathering and length of lather, a combination of ammonium laurel sulfate and ammonium laureth sulfate is particularly preferred. Addition of cocomonoethanol amide to ammonium laurel sulfate to the cleansing phase increases the lather. Behenyl alcohol may be combined with the cleansing surfactant to improve lather quality similar to the effects of adding small amounts of free fatty acid.

In some embodiments, the cleansing surfactant system optionally comprises sarcosinates and sarcosine derivatives as detersive surfactant. As used herein, sarcosinates are the derivatives of sarcosine and N-methyl glycine, acylated with a fatty acid chloride. In some embodiments, the sarcosinate is selected from sodium lauryl sarcosinate, lauryl sarcosine, cocoyl sarcosine, and combination thereof. In some embodiments, the sarcosinate is sodium lauryl sarcosinate.

The amount of the anionic surfactant component in the silk personal care composition should be sufficient to provide the desired cleaning and lather performance, and generally range from about 5.0 wt. % to about 50.0 wt. % by the total weight of the silk personal care composition. In some embodiments, the amount of the anionic surfactant component in the silk personal care composition ranges from about 8.0 wt. % to about 30.0 wt. % by the total weight of the silk personal care composition. In some embodiments, the amount of the anionic surfactant component in the silk personal care composition ranges from about 10.0 wt. % to about 25.0 wt. % by the total weight of the silk personal care composition. In some embodiments, the amount of the anionic surfactant component in the silk personal care composition ranges from about 12.0 wt. % to about 22.0 wt. % by the total weight of the silk personal care composition.

In some embodiments, the cleansing surfactant system comprises zwitterion or amphoteric detersive surfactants as detersive surfactant. Amphoteric detersive surfactants suitable for use in the personal care composition include surfactants broadly described as derivatives of aliphatic secondary and tertiary amines in which the aliphatic chain can be straight or branched chains with at least one having from about 8 to about 18 carbon atoms and at least one having an anionic group including carboxyl, sulfonate, sulfate, phosphate, or phosphonate.

In some embodiments, the amphoteric detersive surfactants are selected from the group consisting of cocoampho acetate, cocoamphodiacetate, lauroamphoacetate, lauroamphodiacetate, cocobetaine and cocamidopropyl betaine, monoacetates (e.g. sodium lauroamphoacetate), diacetates (e.g. disodium lauroamphoacetate), amino- and alkylamino-propionates (e.g. lauraminopropionic acid), sultaines (also as sulfobetaines), cocamidopropylhydroxysultaine, and combinations thereof.

In some embodiments, the total weight of the silk personal care composition comprises about 0.5 wt. % to about 20.0 wt. % of the amphoteric detersive surfactant. In some embodiments, the total weight of the silk personal care composition comprises about 1.0 wt. % to about 10.0 wt. % of the amphoteric detersive surfactant. Additional examples of suitable zwitterion or amphoteric surfactants can be found in U.S. Pat. Nos. 5,104,646 and 5,106,609.

In some embodiments, the cleansing surfactant system may further a single surfactant in addition to silk fibroin protein fragments, e.g., an anionic surfactant to provide foam (e.g., sodium lauryl ether sulphate). In some embodiments, the cleansing surfactant system may further a mixture of sodium lauryl ether sulphate and cocoamidopropyl betaine in addition to silk fibroin protein fragments.

In some embodiments, in addition to silk fibroin protein fragments, the cleaning composition comprises a detersive surfactant selected from the group consisting of sodium lauryl sulfate, sodium laureth sulfate, disodium lauryl sulfosuccinate, cocoyl sarcosinate, cocoamphocarboxy glycinate and cocobetaine, and combination thereof.

In some embodiments, in addition to silk fibroin protein fragments, the cleaning composition comprises a non-sulfate surfactant selected from the group consisting of Cannabis sativa seed oil PEG-8 esters, sodium lauryl oat amino acid, sodium cocoyl glutamate, sodium cocoyl hydrolyzed, amaranth protein, disodium sulfosuccinate lauryl glucoside cross-polymer, hydrolyzed oat protein, sodium cocoyl apple amino acids, decyl glucoside, lauryl glucoside, arachidyl glucoside, caprylyl/capryl glucoside, coco-glucoside, sweet almond amphoacetate, saponins, betaine, and combination thereof.

In some embodiments, the cleansing composition further comprises alkyl polyglucoside. In some embodiments, alkyl polyglucoside is selected from the group consisting of decyl glucoside, lauryl glucoside, arachidyl glucoside, caprylyl/capryl glucoside, coco-glucoside, and combinations thereof. In some embodiments, alkyl polyglucoside is caprylyl/capryl glucoside.

In some embodiments, the cleansing composition further comprises alkyl polyglucoside and a carbohydrate binding protein, wherein alkyl polyglucoside is selected from the group consisting of decyl glucoside, lauryl glucoside, arachidyl glucoside, caprylyl/capryl glucoside, coco-glucoside, and combinations thereof, and the carbohydrate binding protein is galectin selected from the group consisting of Gal-4, Gal-8, Gal-7, and Gal-9. Typically, the galectin comprises a C-terminal carbohydrate recognition domain.

In some embodiments, the cleansing phase comprises the alkyl glucoside and silk fibroin protein fragments in a ratio of glucoside to silk fibroin at 1:5 w/w. In some embodiments, the cleansing phase comprises the alkyl glucoside and silk fibroin protein fragments in a weight ratio of glucoside to silk fibroin selected from the group consisting of 1:5, 1:6, 1:7, 1:8, 1:9, 1:10, 1:11, 1:12, 1:13, 1:14, 1:15, 1:16, 1:17, 1:18, 1:19, and 1:20. In some embodiments, the cleansing phase comprises the alkyl glucoside and silk fibroin protein fragments, wherein alkyl glucoside is presented in the silk personal care composition in an amount of about 1.0 wt. % and silk fibroin protein fragments are presented at an amount of about 5.0 wt. % by the total weight of the silk personal care composition.

In some embodiments, the cleansing phase comprises the alkyl glucoside and silk fibroin protein fragments in a ratio of alkyl glucoside to silk fibroin at a value of about 1:5 to about 1:11. In some embodiments, the cleansing phase comprises the alkyl glucoside and silk fibroin protein fragments in a ratio of alkyl glucoside to silk fibroin of about 1:5. In some embodiments, the cleansing phase comprises the alkyl glucoside and silk fibroin protein fragments in a ratio of alkyl glucoside to silk fibroin of about 1:11. In some embodiments, the cleansing phase comprises the alkyl glucoside and silk fibroin protein fragments in a ratio of about 99:1, about 98:2, about 97:3, about 96:4, about 95:5, about 94:6, about 93:7, about 92:8, about 91:9, about 90:10, about 89:11, about 88:12, about 87:13, about 86:14, about 85:15, about 84:16, about 83:17, about 82:18, about 81:19, about 80:20, about 79:21, about 78:22, about 77:23, about 76:24, about 75:25, about 74:26, about 73:27, about 72:28, about 71:29, about 70:30, about 69:31, about 68:32, about 67:33, about 66:34, about 65:35, about 64:36, about 63:37, about 62:38, about 61:39, about 60:40, about 59:41, about 58:42, about 57:43, about 56:44, about 55:45, about 54:46, about 53:47, about 52:48, about 51:49, about 50:50, about 49:51, about 48:52, about 47:53, about 46:54, about 45:55, about 44:56, about 43:57, about 42:58, about 41:59, about 40:60, about 39:61, about 38:62, about 37:63, about 36:64, about 35:65, about 34:66, about 33:67, about 32:68, about 31:69, about 30:70, about 29:71, about 28:72, about 27:73, about 26:74, about 25:75, about 24:76, about 23:77, about 22:78, about 21:79, about 20:80, about 19:81, about 18:82, about 17:83, about 16:84, about 15:85, about 14:86, about 13:87, about 12:88, about 11:89, about 10:90, about 9:91, about 8:92, about 7:93, about 6:94, about 5:95, about 4:96, about 3:97, about 2:98, or about 1:99.

In some embodiments, the alkyl glucoside is presented in the silk personal care composition in an amount ranging from about 0.3 wt. % to about 6.0 wt. %. In some embodiments, the alkyl glucoside is presented in the silk personal care composition in an amount in a range of about 0.5 wt. % to about 1 wt. %. In some embodiments, the alkyl glucoside is presented in the silk personal care composition in an amount selected from the group consisting of about 0.3 wt. %, about 0.4 wt. %, about 0.5 wt. %, about 0.6 wt. %, about 0.7 wt. %, about 0.8 wt. %, about 0.9 wt. %, and about 1.0 wt. %. about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, and about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, and about 6.0 wt. %. In some embodiments, the alkyl glucoside is present in the silk personal care composition in an amount of about 0.5 wt. %. In some embodiments, the alkyl glucoside is present in the silk personal care composition in an amount of about 1 wt. %.

In some embodiments, the silk personal care composition comprises the silk fibroin protein fragments at an amount ranging from about 0.5 wt. % to about 6.0 wt. %. In some embodiments, the silk personal care composition comprises the silk fibroin protein fragments at an amount selected from the group consisting of about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, and about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, and about 6.0 wt. %.

In some embodiments, the alkyl glucoside is present in an amount ranging from about 0.5% w/w, w/v or v/v to about 1.0% w/w, w/v or v/v of and the silk fibroin protein fragments is present in an amount ranging from about 5.0% w/w, w/v or v/v to about 5.5 w/w, w/v or v/v by the basis of the cleansing phase. In some embodiments, the alkyl glucoside is present in an amount of about 0.5% w/w and the silk fibroin protein fragment is present in an amount of about 5.5% w/w by the basis of the cleansing phase. In some embodiments, the alkyl glucoside is present in an amount of about 1% w/w and the silk fibroin protein fragment is present in an amount of about 5.0 w/w by the basis of the cleansing phase.

In some embodiments, the alkyl glucoside is caprylyl/capryl glucoside. In some embodiments, the cleansing phase comprises from about 0.2% w/w, w/v or v/v to about 1.0% w/w, w/v or v/v of caprylyl/capryl glucoside and about 1.0% w/w, w/v or v/v to about 5.0% w/w, w/v or v/v of the silk fibroin fragments. In some embodiments, the cleansing phase comprises from about 0.5% w/w, w/v or v/v to about 1.0% w/w, w/v or v/v of caprylyl/capryl glucoside and about 5.0% w/w, w/v or v/v to about 5.5% w/w, w/v or v/v of the silk fibroin fragments. In some embodiments, the cleansing phase comprises from about 0.5 w/w of caprylyl/capryl glucoside and about 5.5% w/w of the silk fibroin fragments. In some embodiments, the cleansing phase comprises from about 1.0% w/w of caprylyl/capryl glucoside and about 5.0% w/w of the silk fibroin fragments. In some embodiments, the cleansing phase comprises the caprylyl/capryl glucoside and silk fibroin fragments in a weight ration at a value selected from about 1:5 to about 1:11. In some embodiments, the cleansing phase comprises caprylyl/capryl glucoside and silk fibroin fragments in a weight ratio of about 1:5. In some embodiments, the cleansing phase comprises caprylyl/capryl glucoside and silk fibroin fragments in a weight ratio of about 1:11.

In some embodiments, the total weight amount of cleansing surfactant in the cleansing phase ranges from about 1.0 wt. % to about 50.0 wt. % by the total weight of the silk personal care composition. In some embodiments, the total weight amount of cleansing surfactant in the cleansing phase ranges from about 2.0 wt. % to about 40.0 wt. % by the total weight of the silk personal care composition. In some embodiments, the total weight amount of cleansing surfactant in the cleansing phase ranges from about 10.0 wt. % to about 25.0 wt. % by the total weight of the silk personal care composition. In some embodiments, the total weight amount of cleansing surfactant in the cleansing phase is selected from the group consisting of about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, about 11.0 wt. %, about 12.0 wt. %, about 13.0 wt. %, about 14.0 wt. %, about 15.0 wt. %, about 16.0 wt. %, about 17.0 wt. %, about 18.0 wt. %, about 19.0 wt. %, about 20.0 wt. %, about 21.0 wt. %, about 22.0 wt. %, about 23.0 wt. %, about 24.0 wt. %, about 25.0 wt. %, about 26.0 wt. %, about 27.0 wt. %, about 28.0 wt. %, about 29.0 wt. %, about 30.0 wt. %, about 31.0 wt. %, about 32.0 wt. %, about 33.0 wt. %, about 34.0 wt. %, about 35.0 wt. %, about 36.0 wt. %, about 37.0 wt. %, about 38.0 wt. %, about 39.0 wt. %, about 40.0 wt. %, about 41.0 wt. %, about 42.0 wt. %, about 43.0 wt. %, about 44.0 wt. %, about 45.0 wt. %, about 46.0 wt. %, about 47.0 wt. %, about 48.0 wt. %, about 49.0 wt. %, and about 50.0 wt. % by the total weight of the silk personal care composition.

### B. Thickener and Viscosity Modifying Agent

In some embodiments, the cleansing phase further comprises viscosity modifiers and/or thickeners.

In some embodiments, the thickener is selected from the group consisting of carbomer polymers, carboxyvinyl polymer, acrylic copolymers, methyl cellulose, copolymers of lactide and glycolide monomers, ethyl cellulose, hydroxyethyl cellulose, hydroxypropyl cellulose, hydroxypropyl methylcellulose, carrageenan, hydrophobically modified hydroxy-ethyl-cellulose, laponite, cellulose ether, sodium carboxymethylcellulose, sodium carboxymethyl hydroxyethyl cellulose, hydrocolloid, gum karaya, gum arabic, guar, HP guar, agar, alginate, curdlan, gelatin, 3-glucan, guar gum, locust bean gum, pectin, starch, arabinoxylan, xanthan gum, gum tragacanth, and combinations thereof. In some embodiments, the thickener is xanthan gum. In some embodiments, the thickener is carrageenan.

In some embodiments, the thickener is selected from the group consisting of talc, fumed silica, polymeric polyether compound (e.g., polyethylene or polypropylene oxide having MW of 300 da to 1,000,000 Da capped with alkyl or acyl groups containing 1 to about 18 carbon atoms), ethylene glycol stearate, alkanolamides of fatty acids having 16 to 22 carbon atoms, polyethylene glycol distearate, polyacrylic acids (e.g., Carbopol® 420, Carbopol® 488 or Carbopol® 493), cross-linked polymers of acrylic acid, copolymers of acrylic acid with a hydrophobic monomer, copolymers of carboxylic acid-containing monomers and acrylic esters (e.g. Carbopol® 1342), cross-linked copolymers of acrylic acid and acrylate esters, polyacrylic acids cross-linked with polyfunctional agent (e.g., Carbopol® 910, Carbopol® 934, Carbopol® 940, Carbopol® 941 and Carbopol® 980, Ultrez® 10), and combinations thereof.

In some embodiments, the cleansing phase comprises about 0.1 wt. % to about 15.0 wt. % of thickener/viscosity modifying agent by the total weight of the silk personal care composition. In some embodiments, the cleansing phase comprises about 0.1 wt. % to about 10.0 wt. % of thickener/viscosity modifying agent by the total weight of the silk personal care composition. In some embodiments, the cleansing phase comprises about 0.5 wt. % to about 6.0 wt. % of thickener/viscosity modifying agent by the total weight of the silk personal care composition. In some embodiments, the cleansing phase comprises about 0.9 wt. % to about 4.0 wt. % of thickener/viscosity modifying agent by the total weight of the silk personal care composition. In some embodiments, the cleansing phase comprises about 2.0 wt. % of thickener/viscosity modifying agent by the total weight of the silk personal care composition. In some embodiments, the cleansing phase comprising the thickener/viscosity modifying agent at an amount selected from the group consisting of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.25 wt. %, about 1.50 wt. %, about 1.75 wt. %, about 2.0 wt. %, about 2.25 wt. %, about 2.5 wt. %, about 2.75 wt. %, about 3.0 wt. %, about 3.25 wt. %, about 3.5 wt. %, about 3.75 wt. %, about 4.0 wt. %, about 4.25 wt. %, about 4.5 wt. %, about 4.75 wt. %, about 5.0 wt. %, about 5.25 wt. %, about 5.5 wt. %, about 5.75 wt. %, about 6.0 wt. %, about 6.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. %, about 10.1 wt. %, about 10.2 wt. %, about 10.3 wt. %, about 10.4 wt. %, about 10.5 wt. %, about 10.6 wt. %, about 10.7 wt. %, about 10.8 wt. %, about 10.9 wt. %, about 11.0 wt. %, about 11.1 wt. %, about 11.2 wt. %, about 11.3 wt. %, about 11.4 wt. %, about 11.5 wt. %, about 11.6 wt. %, about 11.7 wt. %, about 11.8 wt. %, about 11.9 wt. %, about 12.0 wt. %, about 12.1 wt. %, about 12.2 wt. %, about 12.3 wt. %, about 12.4 wt. %, about 12.5 wt. %, about 12.6 wt. %, about 12.7 wt. %, about 12.8 wt. %, about 12.9 wt. %, about 13.0 wt. %, about 13.1 wt. %, about 13.2 wt. %, about 13.3 wt. %, about 13.4 wt. %, about 13.5 wt. %, about 13.6 wt. %, about 13.7 wt. %, about 13.8 wt. %, about 13.9 wt. %, about 14.0 wt. %, about 14.1 wt. %, about 14.2 wt. %, about 14.3 wt. %, about 14.4 wt. %, about 14.5 wt. %, about 14.6 wt. %, about 14.7 wt. %, about 14.8 wt. %, about 14.9 wt. %, and about 15.0 wt. % by the total weight of the silk personal care composition. In some embodiments, the cleansing phase comprising the thickener/viscosity modifying agent at about 0.5 wt. % by the total weight of the silk personal care composition.

### C. Soap Base

In some embodiments, the silk personal care composition is formulated as soap bar comprising a soap base as the cosmetically acceptable carrier. In some embodiments, the soap base comprises a cleansing surfactant system and a structurant system, wherein the structurant system including (a) from 10.0 wt. % to 45.0 wt. % of a structurant, (b) from 6.0 wt. % to 30.0 wt. % by weight of the soap base of a humectant, and (c) 0 to 15.0 wt. % by weight of the soap base of a filler by the total weight of the soap bar; wherein the structurant system forms a highly extended three-dimensional network.

In some embodiments, the cleansing surfactant system comprises a fatty acid with fatty alkyl chain having 8 to 18 carbon atoms, and about 20.0 wt. % to 70 wt. % of N-acyl sarcosinate by the total weight of the soap base, wherein the fatty acid is selected from the group consisting of stearic acid, myristic acid, palmitic acid and lauric acid, wherein sodium N-acyl sarcosinate is selected from lauryl sarcosinate, cocoyl sarcosinate, myristoyl sarcosinate, oleoyl sarcosinate, stearoyl sarcosinate, and combinations thereof. In some embodiments, the cleansing surfactant system may further comprise sodium cocoyl isethionate.

In some embodiments, the cleansing surfactant system comprises sodium coco glyceryl sulfonate and sodium lauryl sarcosinate in addition to silk fibroin protein fragments. In some embodiments, in addition to silk fibroin protein fragments, the cleansing surfactant system comprises stearic acid, lauric acid, and a base selected from the group consisting of magnesium hydroxide, potassium hydroxide, sodium hydroxide and triethanolamine, wherein the weight ratio of stearic acid to lauric acid ranging from 2:1 to 1:1. In some embodiments, in addition to silk fibroin protein fragments, the cleansing surfactant system comprises sodium stearate and sodium cocoyl sarcosine. In some embodiments, in addition to silk fibroin protein fragments, the cleansing surfactant system comprises acyl isethionates and sodium sarcosinate.

In some embodiments, in addition to silk fibroin protein fragments, the cleansing surfactant system comprises a mixture of a soap and a surfactant selected from a synthetic surfactant, a natural surfactant as described above, and combinations thereof. The term “soap” as used herein refers to salts of fatty acids in particular alkali metal, alkaline metal, or alkanolamine salts of fatty acids containing 6 to 22 carbon atoms, preferably from 12 to 18 carbon atoms. The alkali metal, alkaline metal salts include sodium, potassium and magnesium salts. The alkanolamine salts include mono-, di-, or triethanolamine salts. The soaps may be derived from pure fatty acids or from fatty acid mixtures derived from natural oil such as coconut oil, palm oil, rapeseed oil, peanut oil, tallow and olive oil. Soaps may be made by neutralizing fatty acids including lauric acid (C12), myristic acid (C14), palmitic acid (C16), or stearic acid (C18) with alkali metal hydroxide or carbonate.

In some embodiments, the soap has the fatty acid distribution of coconut oil, tallow, or mixtures thereof. The proportion of fatty acids having at least 12 carbon atoms in coconut oil soap is about 85%. This proportion will be greater when using the mixtures of coconut oil and fats such as tallow, palm oil, or non-tropical nut oils or fats of which the principle chain lengths are C16 and higher. In some embodiments, the soap comprises about 50.0 wt. % or greater of saturated soaps by weight of the fatty acid soap.

In some embodiments, the soap is derived from the hydrolysis of nut oils such as coconut oil and palm kernel oil. In some embodiments, the soap is derived from the hydrolysis of triglyceride oils including tallow, palm oil and palm stearin. In some embodiments, the soap is derived from the hydrolysis of triglyceride oils and fats such as tallow, palm oil, sunflower seed oil and soybean oil. In some embodiments, coconut oil in the soap may be substituted in whole or in part by palm kernel oil, babassu oil, ouricuri oil, tucum oil, cohune nut oil, murumuru oil, jaboty kernel oil, khakan kernel oil, dika nut oil, and ucuhuba butter.

In some embodiments, the cleansing surfactant system comprises a soap produced by saponification of fatty materials containing a mixture of about 10.0 wt. % to about 40.0 wt. % coconut oil, palm kernel oil or other laurics rich oils, and about 60.0 wt. % to about 90.0 wt. % tallow, palm oil, palm stearin or other stearics rich oils, or a combination thereof, wherein the wt. % is by the total weight of the soap.

In some embodiments, the cleansing surfactant system comprises an anionic soap produced by saponification of fatty materials containing about 20.0 wt. % to 30.0 wt. % coconut oil, about 30.0 wt. % to 40.0 wt. % tallow, about 30.0 wt. % to 40.0 wt. % palm kernel oil, about 2.0 wt. % to 5.0 wt. % peanut oil, and about 5.0 wt. % to 10.0 wt. % castor oil, wherein the wt. % is by the total weight of the soap base.

In some embodiments, the co-surfactant is selected from the group consisting of anionic surfactant (non-soap), Zwitterion surfactant, ampholytic surfactant, cationic surfactant, and combinations thereof.

In some embodiments, the non-soap anionic surfactant is selected from the group consisting of C8-C22 alkyl sulfonate, C8-C22 alkyl disulfonate, C8-C22 alkene sulfonate, C8-C22 hydroxyalkane sulfonate, alkyl glyceryl ether sulfonate, alkyl benzene sulfonate, alpha olefin sulfonate, C12-C18 alkyl sulfate, alkyl glyceryl ether sulfates, alpha sulfonated tallow fatty acid, alpha sulfonated methyl tallowate, alkyl sulfosuccinates (including mono- and dialkyl, e.g., C6-C22 sulfosuccinates); alkyl and acyl taurates, alkyl and acyl sarcosinates, sulfoacetates, C8-C22 alkyl phosphates, alkyl phosphate esters and alkoxyl alkyl phosphate esters, acyl lactates or lactylates, C8-C22 monoalkyl succinates and maleates, sulphoacetates, acyl isethionates, and combinations thereof.

In some embodiments, the amphoteric surfactant is selected from the group consisting of amphoacetates, alkyl and alkyl amido betaines, alkyl and alkyl amido sulphobetaines, sodium 3-dodecylamino-propionate, sodium 3-dodecylaminopropane sulphonate, sodium N-2-hydroxydodecyl-N-methyltaurate, N-alkyl taurines and N-higher alkyl aspartic acids, and combinations thereof.

In some embodiments, the Zwitterion surfactant is selected from the group consisting of 3-(N—N-dimethyl-N-hexadecylammonium) propane-1-sulphonate betaine, 3-(dodecylmethyl sulphonium) propane-1-sulphonate betaine, 3-(cetylmethylphosphonium)ethane sulphonate betaine, coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl)carboxy methyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine, coco dimethyl sulfopropyl betaine, stearyl dimethyl sulfopropyl betaine, amido betaine, amidosulfobetaine, cocamidopropyl betaine, cocoamidoethyl betaine and combinations thereof. In some embodiments, the zwitterion surfactant comprises cocamidopropyl betaine.

In some embodiments, the nonionic surfactant is selected from the group consisting of ethoxylated fatty alcohol, steareth-2, steareth-4, steareth-10, steareth-20, steareth-100, polysorbate 20, long chain alkyl glucosides having C8-C22 alkyl groups, decyl glucoside, lauryl glucoside, arachidyl glucoside, caprylyl/capryl glucoside and coco-glucoside, coconut fatty acid monoethanolamide (e.g., cocamide MEA), coconut fatty acid diethanolamide, fatty alcohol ethoxylate (alkylpolyethylene glycol), alkylphenol polyethylene glycol; alkyl mercaptan polyethylene glycol; fatty amine ethoxylate (alkylaminopolyethylene glycol), fatty acid ethoxylate (acylpolyethylene glycol), polypropylene glycol ethoxylate (e.g., Pluronic™ block copolymers by BASF), fatty acid alkylolamide, fatty acid amide polyethylene glycol, N-alkyl-fatty acid amide, N-alkoxypolyhydroxy fatty acid amide, sucrose ester, sorbitol ester; polyglycol ether, and combinations thereof.

In some embodiments, the synthetic surfactant is presented in the soap base at an amount selected from the group consisting of about 5.0 wt. % to about 25.0 wt. %, about 8.0 wt. % to about 25.0 wt. %, about 10.0 wt. % to about 25.0 wt. %, about 10 wt. % to about 20 wt. %, about 20 wt. %, about 5.0 wt. % to about 15.0 wt. %, about 10.0 wt. % to about 15.0 wt. %, 0% to about 10.0 wt. %, and about 2.0 wt. % to about 5.0 wt. % by the total weight of the soap base.

In some embodiments, the structuring system comprises gelling agent or structurant. In some embodiments, the structurant system comprises a polysaccharide structurant selected from the group consisting of carbohydrate, starch, cellulose, and combinations thereof. In some embodiments, the structuring system comprises a structurant selected from the group consisting of adhesive, glue, wax, fatty acid, fatty alcohol, silicone grease, biopolymer adhesive, sulfopolyester, polyvinyl alcohol polymer, and combinations thereof. In some embodiments, the structuring system comprises a structurant selected from the group consisting of hydrogenated oil, hydrogenated soybean oil, stearyl alcohol, behenyl alcohol, wax, petroleum waxes, paraffin, castor wax, ceresine, ozokerite, carnauba, bees wax, candelilla wax, polymethylene wax, polyethylene wax, and combinations thereof.

In some embodiments, the structuring agent is a starch selected from the group consisting of natural starch (from corn, wheat, rice, potato, tapioca, cassava), pregelatinized starch, physically and chemically modified starch, and combinations thereof. The term “natural starch” (also known as raw or native starch) as used herein refers to starch that has not been subjected to further chemical or physical modification apart from steps associated with separation and milling.

In some embodiments, the structuring agent is a cellulose selected from the group consisting of microcrystalline cellulose, hydroxyalkyl alkylcellulose ether, and combinations thereof.

In some embodiments, the weight ratio of starch and/or cellulose to polyol in the soap base is selected from the group consisting of 2:1 to 6:1, 3:1 to 5:1, and 4:1.

In some embodiments, the soap base comprises the structurant at an amount selected from the group consisting of at least 1.0 wt. %, at least 2.0 wt. %, at least 3.0 wt. %, at least 4.0 wt. %, at least 5.0 wt. %, at least 6.0 wt. %, at least 7.0 wt. %, at least 8.0 wt. %, at least 9.0 wt. %, and at least 10.0 wt. % by the total weight of the soap base.

In some embodiments, the structurant system comprises about 1.0 wt. % to about 50.0 wt. % of at least one fatty compound having a melting point higher than body temperature, e.g., higher than 40° C. In some embodiments, the fatty compound is selected from the group consisting of fatty alcohol having 12 to 30 carbon atoms (e.g., myristyl alcohol, 1-pentadecanol, cetyl alcohol, 1-heptadecanol, stearyl alcohol, 1-nonadecanol, arachidyl alcohol, 1-heneicosanol, behenyl alcohol, brassidyl alcohol, lignoceryl alcohol, cetyl alcohol, myricyl alcohol, Guerbet alcohol), fatty acid having 9 to 34 carbon atoms (e.g., myristic acid, pentadecanoic acid, palmitic acid, margaric acid, stearic acid, nonadecanoic acid, arachic acid, behenic acid, oleic acid, lauric acid, 12-hydroxystearic acid), oil, fat, wax, and combinations thereof. In some embodiments, the wax is selected from the group consisting of purcelline, shea butter, cocoa butter, Japan wax, esparto gras wax, cork wax, Guaruma wax, rice shoot wax, ouricury wax, montan wax, sunflower wax, ceresine wax, sugar cane wax, carnauba wax, candelilla wax, lanolin, orange wax, shellac wax, ceresine, ozokerite wax, paraffin wax, vaseline, petrolatum, microcrystalline wax, polyalkylene and polyethylene wax, halowax, hydrogenated jojoba wax, and combinations thereof. In some embodiments, the oil is selected from the group consisting of almond oil, soybean oil, sunflower oil, safflower oil, corn oil, palm kernel oil, canola oil, borage oil, evening primrose oil, grapeseed oil, wheat germ oil, avocado oil, jojoba oil, sesame oil, walnut oil, linseed oil, palm oil, olive oil, macadamia oil, castor oil, rapeseed oil, peanut oil, coconut oil, turnip seed oil, hardened castor oil, peanut oil, soya oil, turnip oil, cotton seed oil, sunflower oil, palm oil, almond oil, corn oil, sesame oil, cocoa butter, shea butter and coconut oil.

In some embodiments, the fatty compound is present in the soap base in an amount ranging from about 5.0 wt. % to about 35.0 wt. % by the total weight of the soap bar. In some embodiments, the fatty compound is present in the soap base in an amount ranging from about 8.0 wt. % to about 20.0 wt. % by the total weight of the soap bar.

In some embodiments, the soap base further comprises a humectant and/or emollient. In some embodiments, the humectant/emollient comprises a polyhydric alcohol selected from the group consisting of glycerol, sorbitol, mannitol, sucrose, glucose, hydrolyzed starch, dextrin, maltodextrin, polyoxyethylene glycol (PEG), polyoxypropylene glycol (PPG), and combinations thereof.

In some embodiments, the polyol is present in the soap base at an amount selected from the group consisting of from about 6.0 wt. % to about 30.0 wt. %, from about 8.0 wt. % to about 20.0 wt. %, from about 8.0 wt. % to about 15.0 wt. % by the total weight of the soap base.

In some embodiments, the filler is a water insoluble inorganic particulate material or a water insoluble organic particulate material. In some embodiments, the filler is selected from the group consisting of starch-derived filler, calcium carbonate, calcite, aragonite, vaterite, amorphous alumina, alumino-silicate, talc, clay, kaolin, sepiolite, palygorskite, and combinations thereof.

In some embodiments, the soap base comprises about 30.0 wt. % to about 72.0 wt. % olive oil, about 5.0 wt. % to about 15.0 wt. % coconut oil, about 5.0 wt. % to about 20.0 wt. % buriti oil, about 5.0 wt. % to about 30.0 wt. % shea butter, about 5.0 wt. % or less of castor oil, about 0 wt. % to about 25.0 wt. % casein, and about 2.0 wt. % or less coco butter or beeswax, wherein the wt. % is by the total weight of the soap base.

In some embodiments, the soap base comprises: (1) about 25 wt. % to about 95 wt. % of one or more cleansing surfactant as described herein, (2) about 1.0 wt. % to about 50.0 wt. % of at least one wax, (3) 0 wt. % to 10.0 wt. % of at least one fatty material selected from fatty alcohol or free fatty acid. The free fatty acid is useful for improving lather, as well as modifying the rheology at low levels to increase soap bar plasticity. In some embodiments, the fatty acids are saturated, straight-chain C12-C18 fatty acids. Some of the naturally sourced C12-C18 fatty acids are derived from coconut oil, palm kernel oil, and babassu oil.

In some embodiments, the soap base further comprises an additive selected from the group consisting of organic amine including isopropyl amine, glycerol, EDTA, filler, binder, colorant, perfume, and combinations thereof

### (2). Benefit Phase

When washing skin with conventional cleansing compositions, the natural skin lipids are removed together with the dirt and unwanted oils. When too much of the natural lipid is removed, for example by especially frequent washing, the skin becomes dry and irritate. Skin conditioning agents have been incorporated into the skin cleansing composition to restore the condition of the skin. It is believed that the conditioning agent provides improved conditioning benefits to the skin, e.g., moisturizing the skin.

In some embodiments, this disclosure provides a skin cleansing composition comprising a conditioning emulsion phase containing the silk fibroin protein derivatives as described above and skin conditioning agent, wherein the conditioning emulsion comprises an aqueous internal phase and an external oil phase. In some embodiments, the skin condition agent include both water soluble conditioning agent (e.g., humectant) and oil soluble conditioning agent (e.g., emollient, lipid). In some embodiments, the conditioning emulsion comprises the emulsion carriers as described above.

### A. Silk Fibroin Protein Fragments as Skin Conditioning Agent

The silk protein in the silk personal care composition can provide skin conditioning benefits such as promoting cell repair and regeneration, reducing transdermal water loss, boosting collagen level, alleviating sun damage, gentle skin exfoliator, skin tightening, improving appearance of scar, and reducing skin inflammation.

In some embodiments, one or more of the silk fibroin protein based fragments, the silk fibroin amino acids and silk peptides are incorporated as functional additives to the silk personal care composition to impart skin conditioning benefits, for example, adding water soluble silk fibroin protein derived peptide having 2-50 amino acid units to a detersive surfactant for skin cleansing composition, silk fibroin hydrolysate as humectant, silk fibroin protein hydrolysate with an average molecular weight of 1000 Da as skin conditioning agent for a skin cleansing composition, amino acids derived from the silk fibroin protein hydrolysate as skin nutrients.

The silk fibroin protein fragments as described herein have adjustable molecular weight and excellent film forming properties and good humectancy. The silk fibroin protein fragments described herein find uses in skin treatment liquids, skin lotion, skin cream, cleansing cream, and soap.

Silk fibroin is composed of strings of amino acids having the same pH as that of skin. Studies show amino acids from silk fibroin can counter the effects of aging in facial skin and can help calm the nervous system. Silk fibroin also contains natural cellular albumen that helps speed up the metabolism of skin cells thus helping to reduce signs of aging. Further, it is well documented that silk fibroin can form a barrier layer on the skin to help retain moisture and having a plumping, anti-wrinkle effect. Silk fibroin is naturally hypoallergenic. Clinical studies have proven that silk fibroin can help ease conditions such as eczema, atopic dermatitis, sensitive skin, allergic rash, psoriasis, post-chemotherapy sensitive skin, physiological skin flora. Silk fibroin is a natural heat and moisture regulator due to the presence of the hydrophilic functional groups —NH₂ (arginine), —OH (serine), and —COOH (glutamic acid) from amino acids residue of the silk peptide chain. (http://www.mulberrytreesilk.com/blog/benefits-of-silk; Dixit, Silk in personal care products & cosmetics, hpicindia, 2016, pp. 47-55).

In some embodiments, the silk fibroin amino acids are from commercially available hydrolyzed silk (CAS Number: 96690-41-4). The amino acid composition derived from the silk fibroin protein of Bombyx mori consists mainly of Gly (43%), Ala (30%), and Ser (12%). Glycine is a non-essential amino acid used therapeutically as a nutrient. Glycine is also a fast neurotransmitter inhibitor, important in the generating of hormones responsible for a strong immune system, triggering the release of oxygen to energy for cell-making process. Alanine is a non-essential amino acid that degrades in the liver to produce important biomolecules such as pyruvate and glutamate. Serine is a non-essential amino acid known for assisting in production of immunoglobulin's and antibodies for a healthy immune system. Serine is also known for helping the absorption of creatine that helps build and maintain the muscles.

In some embodiments, the skin-conditioning agent comprises low molecular weight silk fibroin peptides have a weight average molecular weight selected from between about 200 Da to about 4 kDa. In some embodiments, the skin-conditioning agent comprises the silk fibroin-based protein fragments have a weight average molecular weight selected from between about 200 Da to about 1 kDa. In some embodiments, the skin conditioning agent comprises the silk fibroin-based protein fragments have a weight average molecular weight of about 1 kDa. In some embodiments, the skin conditioning agent comprises the silk fibroin-based protein fragments have a weight average molecular weight selected from between about 14 kDa to about 30 kDa.

In some embodiments, the skin-conditioning agent comprises the silk fibroin-based protein fragment having a weight average molecular weight selected from between about 5 kDa to about 17 kDa, wherein the silk fibroin-based protein fragments have a polydispersity ranging from about 1.5 to about 3.0.

In some embodiments, the personal care composition comprises silk fibroin derived conditioning agent at an amount ranges from about 0.2 wt. % to about 0.6 wt. %. In some embodiments, the personal care composition comprises silk fibroin derived conditioning agent at an amount selected from the group consisting of about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, about 0.5 wt. %, about 0.51 wt. %, about 0.52 wt. %, about 0.53 wt. %, about 0.54 wt. %, about 0.55 wt. %, about 0.56 wt. %, about 0.57 wt. %, about 0.58 wt. %, about 0.59 wt. %, and about 0.6 wt. %.

In some embodiments, the disclosure provides the following non-limiting soap formulas. All combinations and concentrations of components, including concentrations of silk solutions, can be used as described herein, and any component can be substituted with other similar components described herein. All molecular weights and polydispersities of silk fibroin fragments described herein can be used. The limitation of any formula, such as an ingredient amount or range, can be combined with the limitation(s) of any other formula.

Soap Formula 100

| Ingredient | Wt % |
|---|---|
| Caprylyl/capryl glucoside (may be 100% or less active | 0.01-0.08 |
| material; in some embodiments, is between 60-70% active) | |
| Activated Silk (1-15% soln; low, medium, and/or high | 0.10-1.00 |
| molecular weight) | |
| Cocobetaine | 2.50-12.50 |
| Decyl glucoside | 0.50-2.50 |
| Aspen bark extract | 0.10-2.00 |
| Dermosoft anisate | 0.10-1.50 |
| Natrosol 250 HHR CS | 0.10-2.00 |
| 1,3-propanediol | 0.10-2.00 |
| about 50/50 citric acid | 0.001-0.25 |
| Water | Balance |
| | to 100 |

Soap Formula 101

| Ingredient | Wt % |
|---|---|
| Caprylyl/capryl glucoside (may be 100% or less active | 0.001-0.10 |
| material; in some embodiments, is between 60-70% active) | |
| Activated Silk (about 6% soln; MW/PDI herein) | about 0.59 |
| Cocobetaine | about 7.50 |
| Decyl glucoside | about 1.80 |
| Aspen bark extract | about 0.90 |
| Dermosoft anisate | about 0.50 |
| Natrosol 250 HHR CS | about 0.85 |
| 1,3-propanediol | about 0.90 |
| about 50/50 citric acid | about 0.05 |
| Water | Balance |
| | to 100 |

Soap Formula 102

| Ingredient | Wt % |
|---|---|
| Caprylyl/capryl glucoside (may be 100% or less active | about 0.04 |
| material; in some embodiments, is between 60-70% active) | |
| Activated Silk (about 6% soln; MW/PDI herein) | 0.01-5.00 |
| Cocobetaine | about 7.50 |
| Decyl glucoside | about 1.80 |
| Aspen bark extract | about 0.90 |
| Dermosoft anisate | about 0.50 |
| Natrosol 250 HHR CS | about 0.85 |
| 1,3-propanediol | about 0.90 |
| about 50/50 citric acid | about 0.05 |
| Water | Balance |
| | to 100 |

Soap Formula 103

| Ingredient | Wt % |
|---|---|
| Caprylyl/capryl glucoside (may be 100% or less active | about 0.04 |
| material; in some embodiments, is between 60-70% active) | |
| Activated Silk (about 6% soln; MW/PDI herein) | about 0.59 |
| Cocobetaine | 1.00-15.00 |
| Decyl glucoside | about 1.80 |
| Aspen bark extract | about 0.90 |
| Dermosoft anisate | about 0.50 |
| Natrosol 250 HHR CS | about 0.85 |
| 1,3-propanediol | about 0.90 |
| about 50/50 citric acid | about 0.05 |
| Water | Balance |
| | to 100 |

Soap Formula 104

| Ingredient | Wt % |
|---|---|
| Caprylyl/capryl glucoside (may be 100% or less active | about 0.04 |
| material; in some embodiments, is between 60-70% active) | |
| Activated Silk (about 6% soln; MW/PDI herein) | about 0.59 |
| Cocobetaine | about 7.50 |
| Decyl glucoside | 0.10-5.00 |
| Aspen bark extract | about 0.90 |
| Dermosoft anisate | about 0.50 |
| Natrosol 250 HHR CS | about 0.85 |
| 1,3-propanediol | about 0.90 |
| about 50/50 citric acid | about 0.05 |
| Water | Balance |
| | to 100 |

Soap Formula 105

| Ingredient | Wt % |
|---|---|
| Caprylyl/capryl glucoside (may be 100% or less active | about 0.04 |
| material; in some embodiments, is between 60-70% active) | |
| Activated Silk (about 6% soln; MW/PDI herein) | about 0.59 |
| Cocobetaine | about 7.50 |
| Decyl glucoside | about 1.80 |
| Aspen bark extract | 0.01-5.00 |
| Dermosoft anisate | about 0.50 |
| Natrosol 250 HHR CS | about 0.85 |
| 1,3-propanediol | about 0.90 |
| about 50/50 citric acid | about 0.05 |
| Water | Balance |
| | to 100 |

Soap Formula 106

| Ingredient | Wt % |
|---|---|
| Caprylyl/capryl glucoside (may be 100% or less active | about 0.04 |
| material; in some embodiments, is between 60-70% active) | |
| Activated Silk (about 6% soln; MW/PDI herein) | about 0.59 |
| Cocobetaine | about 7.50 |
| Decyl glucoside | about 1.80 |
| Aspen bark extract | about 0.90 |
| Dermosoft anisate | 0.01-5.00 |
| Natrosol 250 HHR CS | about 0.85 |
| 1,3-propanediol | about 0.90 |
| about 50/50 citric acid | about 0.05 |
| Water | Balance |
| | to 100 |

Soap Formula 107

| Ingredient | Wt % |
|---|---|
| Caprylyl/capryl glucoside (may be 100% or less active | about 0.04 |
| material; in some embodiments, is between 60-70% active) | |
| Activated Silk (about 6% soln; MW/PDI herein) | about 0.59 |
| Cocobetaine | about 7.50 |
| Decyl glucoside | about 1.80 |
| Aspen bark extract | about 0.90 |
| Dermosoft anisate | about 0.50 |
| Natrosol 250 HHR CS | 0.01-5.00 |
| 1,3-propanediol | about 0.90 |
| about 50/50 citric acid | about 0.05 |
| Water | Balance |
| | to 100 |

Soap Formula 108

| Ingredient | Wt % |
|---|---|
| Caprylyl/capryl glucoside (may be 100% or less active | about 0.04 |
| material; in some embodiments, is between 60-70% active) | |
| Activated Silk (about 6% soln; MW/PDI herein) | about 0.59 |
| Cocobetaine | about 7.50 |
| Decyl glucoside | about 1.80 |
| Aspen bark extract | about 0.90 |
| Dermosoft anisate | about 0.50 |
| Natrosol 250 HHR CS | about 0.85 |
| 1,3-propanediol | 0.01-5.00 |
| about 50/50 citric acid | about 0.05 |
| Water | Balance |
| | to 100 |

Soap Formula 109

| Ingredient | Wt % |
|---|---|
| Caprylyl/capryl glucoside (may be 100% or less active | about 0.04 |
| material; in some embodiments, is between 60-70% active) | |
| Activated Silk (about 6% soln; MW/PDI herein) | about 0.59 |
| Cocobetaine | about 7.50 |
| Decyl glucoside | about 1.80 |
| Aspen bark extract | about 0.90 |
| Dermosoft anisate | about 0.50 |
| Natrosol 250 HHR CS | about 0.85 |
| 1,3-propanediol | about 0.90 |
| about 50/50 citric acid | 0.001-0.50 |
| Water | Balance |
| | to 100 |

In some embodiments, the disclosure provides the following non-limiting hand sanitizer formulas. All combinations and concentrations of components, including concentrations of silk solutions, can be used as described herein, and any component can be substituted with other similar components described herein. All molecular weights and polydispersities of silk fibroin fragments described herein can be used. The limitation of any formula, such as an ingredient amount or range, can be combined with the limitation(s) of any other formula.

Hand Sanitizer Formula 200

| Ingredient | Wt % |
|---|---|
| Activated Silk (1-15% soln; low, medium, and/or high | 0.10-2.50 |
| molecular weight) | |
| Hydroxypropylcellulose | 0.10-1.50 |
| Water | 10.00-35.00 |
| Ethanol | Balance |
| | to 100 |

Hand Sanitizer Formula 201

| Ingredient | Wt % |
|---|---|
| Activated Silk (about 6% soln; MW/PDI herein) | 0.05-5.00 |
| Hydroxypropylcellulose | about 0.5 |
| Water | about 28.2 |
| Ethanol | Balance |
| | to 100 |

Hand Sanitizer Formula 202

| Ingredient | Wt % |
|---|---|
| Activated Silk (about 6% soln; MW/PDI herein) | about 1.0 |
| Hydroxypropylcellulose | 0.01-5.00 |
| Water | about 28.2 |
| Ethanol | Balance |
| | to 100 |

Hand Sanitizer Formula 203

| Ingredient | Wt % |
|---|---|
| Activated Silk (about 6% soln; MW/PDI herein) | about 1.0 |
| Hydroxypropylcellulose | about 0.5 |
| Water | 5.00-50.00 |
| Ethanol | Balance |
| | to 100 |

### B. Plant Extracts

In some embodiments, the silk personal care composition optionally comprises plant extract that enhances the beneficial effects of silk fibroin protein derivatives. In some embodiments, the plant extract is selected from the group consisting of extracts from microbial exopolysaccharide, rice, oat, almond, Camellia sinensis (green tea) extract, Butyrospermum Parkii (shea butter), coconut, papaya, mango, peach, lemon, wheat, rosemary, apricot, algae, grapefruit, sandalwood, lime, orange, Acacia concinna, Butea parviflora, Butea superb, Butea frondosa, Campanulata (fire tulip), Adansonia Digitata (Baobab), Phoenix Dactylifera (date), Hibiscus Sabdariffa (hibiscus), Aframomum Melegueta (African pepper), Khaya senegalensis (mahogany wood), Tamarindus Indica (tamarind, or curcumin), Cyperus Papyrus (papyrus), Ageratum spp., birch, burdock, horsetail, lavender, marjoram, nettle, tail cat, thyme, oak bark, echinacea, stinging nettle, witch hazel, hops, henna, chamomile, whitethorn, lime-tree blossom, almond, pine needles, horse chestnut, juniper, kiwi, melon, mallow, cuckoo flower, wild thyme, yarrow, melissa, rest harrow, coltsfoot, marshmallow, rice meristem, moringa, ginseng and ginger root, aloe vera, aloe barbadensis leaf extract, Lavandula angustifolia (lavender) flower extract, Sambucus nigra (elderberry) fruit extract, Phoenix dactylifera (date) seed extract, Avandula stoechas (Spanish lavender) extract, Spiraea ulmaria (meadowsweet) leave extract, Chamomilla recutita (chamomile) leaf extract, and Symphytum officinale (comfrey) leaf extract, algae polysaccharide, tomato, spinach, carrot, lettuce, bean sprouts, Chinese cabbage, onion, mugwort, allium, eggplant, soybean, cabbage, injinssuk, matricaria, comfrey, cucumber, and combination thereof. The extracts of these plants are obtained from seeds, roots, stem, leaves, flowers, bark, fruits, and/or whole plant. The plant extracts may include aqueous extracts, or oil extract.

In some embodiments, the silk personal care composition comprises about 0.001 wt. % to about 10.0 wt. % of the plant extract. In some embodiments, the silk personal care composition comprises about 0.005 wt. % to about 5.0 wt. % of the plant extract. In some embodiments, the silk personal care composition comprises about 0.01 wt. % to about 2.0 wt. % of the plant extract. In some embodiments, the silk personal care composition comprises 0.0045 wt. % to 0.0055 wt. % of the plant extract.

### C. Emollients

In some embodiments, the silk personal care composition optionally comprises an emollient selected from the group consisting of a hydrocarbon oil, a hydrocarbon wax, a silicone oil, an acetoglyceride ester, an ethoxylated glyceride, an alkyl ester of a fatty acid, an alkenyl ester of a fatty acid, a fatty acid, a fatty alcohol, a fatty alcohol ether, an ether-ester, lanolin, a lanolin derivative, a polyhydric alcohol, a polyether derivative, a polyhydric ester, a wax ester, a beeswax derivative, a vegetable wax, a natural or essential oil, a phospholipid, a sterol, an amide, and combination thereof.

In some embodiments, the emollients incorporated in the silk personal care compositions comprise one or more of (1) hydrocarbon oils and waxes, e.g., mineral oil, petrolatum, paraffin, ozokerite, microcrystalline wax, polyethylene, squalene, and perhydrosqualene; (2) silicone oils, e.g., dimethyl polysiloxanes, methylphenyl polysiloxanes, water-soluble and alcohol-soluble silicone glycol copolymers; (3) acetoglyceride esters, e.g., acetylated monoglycerides; (4) ethoxylated glycerides, e.g., ethoxylated glyceryl monostearate; (5) alkyl esters of fatty acids having 10 to 20 carbon atoms, e.g., hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, methyl, isopropyl, butyl esters of fatty acids; (6) alkenyl esters of fatty acids having 10 to 20 carbon atoms, e.g., oleyl myristate, oleyl stearate, and oleyl oleate; (7) fatty acids having 10 to 20 carbon atoms, e.g., pelargonic, lauric, myristic, palmitic, stearic, isostearic, hydroxystearic, oleic, linoleic, ricinoleic, arachidic, behenic, and erucic acids; (8) fatty alcohols having 10 to 20 carbon atoms, e.g., lauryl, myristyl, cetyl, hexadecyl, stearyl, isostearyl, hydroxystearyl, oleyl, ricinoleyl, behenyl, erucyl alcohols, and 2-octyl dodecanol; (9) fatty alcohols ethers, e.g., ethoxylated fatty alcohols of 10 to 20 carbon atoms, lauryl, cetyl, stearyl, isostearyl, oelyl, and cholesterol alcohols having attached thereto from 1 to 50 ethylene oxide groups or 1 to 50 propylene oxide groups; (10) ether-esters, e.g. fatty acid esters of ethoxylated fatty alcohols; (11) lanolin and its derivatives, e.g., lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, ethoxylated sorbitol lanolin, and liquid and semisolid lanolin absorption bases; (12) polyhydric alcohols and polyether derivatives, e.g., propylene glycol, dipropylene glycol, polypropylene glycols 2000 and 4000, polyoxyethylene glycols, polyoxypropylene polyoxyethylene glycols, glycerol, sorbitol, ethoxylated sorbitol, hydroxypropyl sorbitol, polyethylene glycols 200-6000, methoxy polyethylene glycols 350, 550, 750, 2000 and 5000, poly[ethylene oxide]homopolymers (weight average molecular weight of 100,000-5,000,000 Da), polyalkylene glycols and derivatives, hexylene glycol (2-methyl-2,4-pentanediol), 1, 3-butylene glycol, 1,2,6-hexanetriol, ethohexadiol USP (2-ethyl-1,3-hexanediol), C15-C18 vicinal glycol, and polyoxypropylene derivatives of trimethylolpropane; (13) polyhydric alcohol esters, e.g., ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters, sucrose cocoate, sucrose dilaurate, sucrose distearate, sucrose hexaerucate, sucrose laurate, sucrose myristate, sucrose oleate, sucrose palmitate, sucrose pentaerucate, sucrose polybehenate, sucrose polycottonseedate, sucrose polylaurate, sucrose polylinoleate, sucrose polyoleate, sucrose polypalmate, sucrose polysoyate, sucrose polystearate, sucrose ricinoleate, sucrose stearate, sucrose tetraisostearate, sucrose tribehenate, sucrose tristearat; (14) wax esters, e.g., beeswax, spermaceti, myristyl myristate, and stearyl stearate; (15) beeswax derivatives, e.g., polyoxyethylene sorbitol beeswax which are reaction products of beeswax with ethoxylated sorbitol of varying ethylene oxide content; (16) vegetable waxes, e.g., carnauba and candelilla waxes; (17) natural or essential oils, e.g., citrus oil, non-citrus fruit oil, nut oils, oils having flavors, perfume or scents, canola oil, corn oil, neem oil, olive oil, cottonseed oil, coconut oil, fractionated coconut oil, palm oil, nut oils, safflower oil, sesame oil, soybean oil, peanut oil, almond oil, cashew oil, hazelnut oil, macadamia oil, pecan oil, pine nut oil, pistachio oil, walnut oil, grapefruit seed oil, lemon oil, orange oil, sweet orange oil, tangerine oil, lime oil, mandarin oil, omega 3 oil, flaxseed oil (linseed oil), apricot oil, avocado oil, carrot oil, cocoa butter oil, coconut oil, fractionated coconut oil, hemp oil, papaya seed oil, rice bran oil, shea butter oil, tea tree seed oil, and wheat germ oil, lavender oil, rosemary oil, tung oil, jojoba oil, poppy seed oil, shea butter, castor oil, mango oil, rose hip oil, tall oil chamomile oil, cinnamon oil, citronella oil, eucalyptus oil, fennel seed oil, jasmine oil, juniper berry oil, raspberry seed oil, lavender oil, primrose oil, lemon grass oil, nutmeg oil, patchouli oil, peppermint oil, pine oil, rose oil, rose hip oil, rosemary oil, eucalyptus oil, tea tree oil, rosewood oil, sandalwood oil, sassafras oil, spearmint oil, Ricinus communis (castor) seed oil, wintergreen oil; (18) phospholipids, e.g., lecithin and derivatives; (19) sterols, e.g., cholesterol and cholesterol fatty acid esters; and (20) fatty acid amides, ethoxylated fatty acid amides, and solid fatty acid alkanolamides, (21) lanolin, Therbroma cacao (cocoa) seed butter, petrolatum, Euphorbia cerifera (candelilla) wax, honey, geraniol, menthol, camphor, cetyl esters, mineral oil, salicylic acid, phenol, palmitoyl isoleucine,

### D. Moisturizers

In some embodiments, the silk personal care composition optionally comprises a moisturizer selected from the group consisting of water-soluble, low molecular weight moisturizers, fat-soluble, low molecular weight moisturizers, water-soluble, high molecular weight moisturizers and fat-soluble, high molecular weight moisturizers, humectant, and combination thereof.

In some embodiments, the moisturizer comprises a humectant. As used herein, the term “humectant” refer to a hygroscopic substance used to keep things moist. A humectant attracts and retains the moisture in the air nearby via absorption, drawing the water vapor into or beneath the organism's or object's surface.

In some embodiments, the personal care composition optionally comprises a water-soluble silk fibroin peptide as humectant. The amino peptides derived from the silk fibroin protein fragments can be easily absorbed by skin. In some embodiments, a water-soluble silk fibroin peptide may be added to the silk personal care composition to give an enhanced after use feeling. In some embodiments, amino acids (glycine, alanine, and serine) derived from the silk fibroin protein fragments may be added to the silk personal care composition as a conditioning agent (e.g. to exert excellent condition effects such as moist feel, softness, smoothness, gloss).

In some embodiments, the silk personal care composition may comprise one or more additional humectant selected from the group consisting of honey, aloe vera, aloe vera leaf juice, aloe vera leaf extract, sorbitol, urea, lactic acid, sodium lactate, pyrrolidone carboxylic acid, trehalose, maltitol, alpha-hydroxy acids, sodium pyroglutamate, pyrolidonecarboxylate, N-acetyl-ethanolamine, sodium lactate, isopropanol, polyalkylene glycols (e.g., ethylene glycol, propylene glycol, hexylene glycol, 1,3-butylene glycol, dipropylene glycol, triethylene glycol), 1,3-propanediol, diethylene glycol monoethyl ether, glyceryl coconate, hydroxystearate, myristate, oleate, sodium hyaluronate, hyaluronic acid, chondroitin sulfuric acid, phospholipids, collagen, elastin, ceramides, lecithin sorbitol, OH—(CH₂—CH₂—O)₄—H (PEG-4), and combination thereof.

In some embodiments, the silk personal care composition optionally comprises polyhydric alcohols as moisturizer selected from the group consisting of ethylene glycol, propylene glycol, 1,3 butylene glycol, glycerin, sorbitol, polyethylene glycol, glutamine, mannitol, pyrrolidone-sodium carboxylate, (polymerization degree n=2 or more), polypropylene glycol (polymerization degree n=2 or more), polyglycerin (polymerization degree n=2 or more), lactic acid, lactate, and combination thereof.

In some embodiments, the silk personal care composition optionally comprises fat-soluble, low molecular weight moisturizers selected from the group consisting of cholesterol and cholesterol ester. In some embodiments, the silk personal care composition optionally comprises water-soluble, high molecular weight moisturizers selected from the group consisting of carboxyvinyl polymers, polyaspartate, tragacanth, xanthane gum, methyl cellulose, hydroxymethyl cellulose, hydroxyethyl cellulose, hydroxypropyl cellulose, carboxymethyl cellulose, water-soluble chitin, chitosan and dextrin. In some embodiments, the silk personal care composition optionally comprises fat-soluble, high molecular weight moisturizers selected from the group consisting of polyvinylpyrrolidone-eicosene copolymers, polyvinylpyrrolidone-hexadecene copolymers, nitrocellulose, dextrin fatty acid ester and high molecular silicone.

Additional suitable moisturizers suitable for the silk personal care composition include water-soluble and/or water swellable polymeric moisturizers. In some embodiments, hyaluronic acid, or chitosan is combined with moisturizers to enhance their properties.

In some embodiments, the silk personal care composition comprises about 0.1 wt. % to about 30.0 wt. % of the moisturizer. In some embodiments, the silk personal care composition comprises about 0.5 wt. % to about 25.0 wt. % of the moisturizer. In some embodiments, the silk personal care composition comprises about 1.0 wt. % to about 20.0 wt. % of the moisturizer.

### E. Antimicrobial Agent

In some embodiments, the silk personal care composition further comprises an antimicrobial agent to inhibit the growth of pathogenic or potentially pathogenic bacteria and fungi, or to kill such organisms.

In some embodiments, the silk personal care composition containing the antimicrobial agent is formulated as skin cleansing and moisturizing product. The antimicrobial component of the silk personal care composition also inhibits the growth of spoilage organisms during storage of the product.

In some embodiments, the antimicrobial agent is selected from the group consisting of bacteriostatic agent, bactericidal agent, fungistatic or fungicidal agent, quaternary ammonium salt, and combinations thereof. In some embodiments, the antimicrobial agent is selected from the group consisting of triclosan, sulfur, citric acid, zinc oxide, chlorhexidene digluconate, chlorhexidene acetate, chlorhexidene isethionate, chlorhexidene hydrochloride, enzalkonium chloride, benzethonium chloride, polyhexamethylene biguanide, cetyl puridium chloride, methyl and benzothonium chloride, parachlorometa xylenol, ethanol, propanol, and combinations thereof.

In some embodiments, the silk personal care composition comprises about 0.001 wt. % to about 5.0 wt. % of the antimicrobial agent. In some embodiments, the silk personal care composition comprises about 0.05 wt. % to about 2.0 wt. % % of the antimicrobial agent. In some embodiments, the silk personal care composition comprises about 0.1 wt. % to about 1.0 wt. % of the antimicrobial agent.

### F. Antioxidant

In some embodiments, the silk personal care composition further comprises an antioxidant. In some embodiments, the antioxidant is selected from the group consisting of uric acid, lipoic acid (lipoic acid, Vitamin A (retinol), Vitamin C (ascorbic acid), Vitamin E (tocopherol acetate), ubiquinol (Ubiquinol, Coenzyme Q10, Se (selenium), lycopene, beta-carotene, alpha-carotene, saponins, tannins, glutathione, polyphenols, superoxide dismutase (SOD), catalase, glutathione oxidoreductase, thioredoxin disulfide reductase, ascorbyl palmirate, ascorbyl myristate, ascorbyl stearate, tocopheryl acetate, tocopheryl propionate, tocopheryl butyrate, panthenol, butylated hydroxytoluene (BHT), ascorbyl palmitate, butylated hydroxyanisole, α-tocopherol, phenyl-α-naphthylamine, sodium sulfite, sodium metabisulfite, sodium bisulfite, sodium thiosulfite, sodium formaldehyde sulfoxylate, isoascorbic acid, thioglycerol, thiosorbitol, thiourea, thioglycolic acid, cysteine hydrochloride, 1,4-diazobicyclo-(2,2,2)-octane, hydroquinone, propyl gallate, nordihydroguiaretic acid, emu oil, and combinations thereof.

In some embodiments, the antioxidant is a radical scavenger selected from the group consisting of ascorbic acid (vitamin C) and its salts, tocopherol (vitamin E), tocopherol sorbate, butylated hydroxy benzoic acids and their salts, 6-hydroxy-2,5,7,8-tetramethylchroman-2-carboxylic acid, gallic acid and its alkyl esters, propyl gallate, uric acid and its salts, uric acid alkyl esters, sorbic acid and its salts, ascorbyl esters of fatty acids, amines (e.g., N,N-diethylhydroxylamine, amino-guanidine), sulfhydryl compounds (e.g., glutathione), dihydroxy fumaric acid and its salts, EDTA, BHT, and combinations thereof.

In some embodiments, the antioxidant comprises at least one water-soluble antioxidant and at least one oil soluble antioxidant. In some embodiments, the water soluble antioxidant is selected from the group consisting of ascorbic acid, sodium sulfite, sodium metabisulfite, sodium bisulfite, sodium thiosulfite, sodium formaldehyde sulfoxylate, isoascorbic acid, thioglycerol, thiosorbitol, thiourea, thioglycolic acid, cysteine hydrochloride, 1,4-diazobicyclo-(2,2,2)-octane, and mixtures thereof. In some embodiments, the oil-soluble antioxidants is selected from the group consisting of butylated hydroxytoluene, ascorbyl palmitate, butylated hydroxyanisole, α-tocopherol, phenyl-α-naphthylamine, and mixtures thereof.

In some embodiments, the antioxidant is ascorbic acid. In some embodiments, the antioxidant is vitamin A. In some embodiments, the antioxidant system comprise emu oil.

In some embodiments, the silk personal care composition comprises about 0.0001 wt. % to about 5.0 wt. % of the antioxidant. In some embodiments, the silk personal care composition comprises about 0.01 wt. % to about 1.0 wt. % of the antioxidant.

### G. Humectant

In some embodiments, the silk personal care composition further comprises one or more humectant. The humectant is a water-soluble component, i.e., it is primarily present in the aqueous phase. The humectant used herein provide stability to the water phase, however it may also provide other functions, such as promotion of water retention by the skin or hair, emolliency, and other moisturizing or conditioning functions.

In some embodiments, the humectant is selected from the group consisting of polyhydric alcohol, C3-C6 diol and triol, polyethylene glycol, propylene glycol, dipropyleneglycol, hexylene glycol, 1,4-dihydroxyhexane, 1,2,6-hexanetriol, sorbitol, butylene glycol, propanediols, such as methyl propane diol, dipropylene glycol, triethylene glycol, glycerin (glycerol), polyethylene glycols, ethoxydiglycol, polyethylene sorbitol, glycolic acid, glycolate salts, lactate salts, lactic acid, sodium pyrrolidone carboxylic acid, hyaluronic acid, chitin and combinations thereof.

In some embodiment, the silk personal care composition comprises about 2.0 wt. % to about 15.0 wt. % of the humectant. In some embodiments, the silk personal care composition comprises about 2.0 wt. % to about 10.0 wt. % of the humectant. In some embodiment, the silk personal care composition comprises about 5.0 wt. % to about 10.0 wt. % of the humectant.

### H. Perfume and Essential Oil

In some embodiments, the silk personal care composition further comprises one or more natural fragrances. In some embodiments, the fragrances comprises natural scents are added to the silk personal care compositions to impart a pleasant, mild scent, and are formulated to avoid any negative impact on the skin such as drying, irritation or allergies. The natural scents may be obtained from plant materials in the form of essential oils.

In some embodiments, the fragrance is selected from the group consisting of essential oil of rose, ylang, lavender, chamomile, citronella, geranium, rosemary, anise seeds, elemi, orris, orange, galbanum, clary sage, clove, coriander, sandalwood, cinnamon, jasmine, spearmint, cedar woods, celery, tangerine, tonka beans, neroli, violet, patchouli, peach, vetiver, petitgrain, peppermint, Peru balsam, bergamot, eucalyptus, lilac, raspberry, lavender, lily-of-the-valley, lemon, lemon grass, lime, amber, castrium, civet, musk, and combinations thereof.

In some embodiments, the silk personal care composition may further comprises a synthetic fragrance selected from the group consisting of 2-methylundecanal, pinene, limonene, caryophyllene, longifolene, myrcene, cis-3-hexenol, levosandol, p-t-butyrocyclohexanol, citronellol, geraniol, nerol, linalol, dihydrolinalol, tetrahydrolinalol, dihydromyrcenol, tetrahydromyrcenol, menthol, terpineol, borneol, isoborneol, isocamphylcyclohexanol, farnesol, cedrol, benzylalcohol, α-phenylethylalcohol, β-phenylethylalcohol, phenoxyethylalcohol, cinnamic alcohol, amylcinnamic alcohol, thymol, eugenol, cineol, estragol, β-naphtholmethyether, β-naphtholethyether, diphenyloxide, cedrolmethyether, isoamylphenylethylether, ambroxan, rose oxide, dihydrorose oxide, limonene oxide, menthofuran, amber core, cis-jasmone, dihydrojasmin, methyldihydrojasmonate, cyclotene, damascenone, damascone, dynascone, ionone, methylionone, irone, cashmeran, carvon, menthone, acetylcedrene, isolongifolanone, raspberry ketone, acetophenone and benzophenone, γ-undecalactone, coumarin, linalyl formate, citronellyl formate, linalyl acetate, citronellyl acetate, geranyl acetate, terpinyl acetate, cedryl acetate, p-t-butylcyclohexyl acetate, benzyl acetate, phenylethyl acetate, styrallyl acetate, isoamyl acetate, rosephenone, dimethylbenzylcarbinyl acetate, jasmal, benzyl benzoate, benzyl salicylate, methyl atrarate, methyl anthranilate, dimethyl anthranilate, ethyl anthranilate, auranthiol, ethyl trimethylcycohexanoate, muscone, muscol, civetone, cyclopentadecanone, cyclohexadecanone, cyclohexadecenone, cyclopentadecanolide, 10-oxahexadecanolide, ethylene brassylate, ethylenedodecanedioate, celestolide, galaxolide, traseolide, phantolide, and combinations thereof.

In some embodiments, the silk personal care composition is a cream comprises about 0.01 wt. % to about 5.0% of the fragrance. In some embodiments, the silk personal care composition is a milky lotion comprises about 0.01 wt. % to about 4.0 wt. % of the fragrance. In some embodiments, the silk personal care composition is a skin cleansing composition comprises about 0.01 wt. % to about 1.0% of the fragrance.

In some embodiments, the fragrance and essential oils described above are encapsulated within a silk fibroin protein fragment particle, wherein the silk fibroin protein fragment forms the matrix or forms a particle shell, the fragrance or essential oils may be embedded with the particle matrix or enclosed inside particle shell as an oil phase or an aqueous phase.

In some embodiments, various emulsion based particle preparation methods reported in the art such as emulsion/evaporation method may be used to prepare silk fibroin protein fragment particles encapsulating essential oil. In some embodiments, the silk solution or the various silk fibroin protein fragments compositions as described above can be used to prepare the silk fibroin protein fragment particle encapsulated perfume/fragrance.

In some embodiments, the silk fibroin protein fragment particles may be nanoparticles or microparticles. In some embodiments, the particle has a median particle size less than 1000 nm. In some embodiments, the median particle size ranges from about 1 nm to about 1000 nm. In some embodiments, the median particle size ranges from about 1 nm to about 500 nm. In some embodiments, the median particle size ranges from about 1 nm to about 250 nm. In some embodiments, the median particle size ranges from about 1 nm to about 150 nm. In some embodiments, the median particle size ranges from about 1 nm to about 100 nm. In some embodiments, the median particle size ranges from about 1 nm to about 50 nm. In some embodiments, the median particle size ranges from about 1 nm to about 25 nm. In some embodiments, the median particle size ranges from about 1 nm to about 10 nm. In some embodiments, the particle has a median particle size of 500 nm. In some embodiments, the particle has a median particle size of 250 nm. In some embodiments, the particle has a median particle size of 750 nm.

In some embodiments, the particles are microparticles having a median particle size equal or greater than 1000 nm (1 micron). In order to achieve good deposition onto skin and a stable formulation, the particles have a median particle size ranging from about 1 μm to about 10.0 μm. In some embodiments, the particles have a median particle size ranging from about 2 to about 50 μm. In some embodiments, the particles have a median particle size ranging from about 2 μm to about 20 μm. In some embodiments, the particles have a median particle size ranging from about 4 μm to about 10 μm. In some embodiments, the particles have a median particle size selected from: about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.1 μm, about 2.2 μm, about 2.3 μm, about 2.4 μm, about 2.5 μm, about 2.6 μm, about 2.7 μm, about 2.8 μm, about 2.9 μm, about 3.0 μm, about 3.1 μm, about 3.2 μm, about 3.3 μm, about 3.4 μm, about 3.5 μm, about 3.6 μm, about 3.7 μm, about 3.8 μm, about 3.9 μm, about 4.0 μm, about 4.1 μm, about 4.2 μm, about 4.3 μm, about 4.4 μm, about 4.5 μm, about 4.6 μm, about 4.7 μm, about 4.8 μm, about 4.9 μm, about 5.0 μm, about 5.1 μm, about 5.2 μm, about 5.3 μm, about 5.4 μm, about 5.5 μm, about 5.6 μm, about 5.7 μm, about 5.8 μm, about 5.9 μm, about 6.0 μm, about 6.1 μm, about 6.2 μm, about 6.3 μm, about 6.4 μm, about 6.5 μm, about 6.6 μm, about 6.7 μm, about 6.8 μm, about 6.9 μm, about 7.0 μm, about 7.1 μm, about 7.2 μm, about 7.3 μm, about 7.4 μm, about 7.5 μm, about 7.6 μm, about 7.7 μm, about 7.8 μm, about 7.9 μm, about 8.0 μm, about 8.1 μm, about 8.2 μm, about 8.3 μm, about 8.4 μm, about 8.5 μm, about 8.6 μm, about 8.7 μm, about 8.8 μm, about 8.9 μm, about 9.0 μm, about 9.1 μm, about 9.2 μm, about 9.3 μm, about 9.4 μm, about 9.5 μm, about 9.6 μm, about 9.7 μm, about 9.8 μm, about 9.9 μm, and about 10.0 μm.

The encapsulated fragrance or essential oil imparts to the silk personal care product many advantageous properties including enhanced compatibility with formulation ingredients, long last scent, reduced toxicity, and increased affinity to skin surface.

### (3) Optional Additive

In some embodiments, the silk personal care composition may comprise an optional additive. In some embodiments, the optional additive is selected from the group consisting of oil absorbent (hydroxyapatite), abrasive, anti-acne agent, anticaking agent, antifoaming agent, antioxidant, thickener, binder, biological additive, buffering agent, bulking agent, chelating agent, chemical additive, colorant, cosmetic biocide, denaturant, cosmetic astringent, reducing agent, sequestrant, external analgesic, clove oil, menthol, camphor, eucalyptus oil, eugenol, menthyl lactate, witch hazel distillate, anti-acne agent, film former, fragrance, opacifying agent, pH adjuster, plasticizer, preservative, propellant, reducing agent, skin protectant, solvent, suspending agent (nonsurfactant), ultraviolet light absorber, and viscosity increasing agent (aqueous and nonaqueous), antimicrobial agent, antibiotic, antifungal, retinoid, insecticide, skin bleaching and lightening agent (e.g., hydroquinone, kojic acid, ascorbic acid, magnesium ascorbyl phosphate, ascorbyl glucosamine), allantoin, bisabolol, dipotassium glycyrrhizinate, and combinations thereof.

In some embodiments, the silk personal care composition may comprise an anti-wrinkle/anti-atrophy active agent selected from the group consisting of sulfur-containing D and L amino acid and derivatives and salts thereof; N-acetyl derivatives of sulfur-containing D and L amino acid; thiol; hydroxy acid (e.g., alpha-hydroxy acids such as lactic acid and glycolic acid and their derivatives and salts, beta-hydroxy acids such as salicylic acid and salicylic acid salts and derivatives), urea, hyaluronic acid, phytic acid, lipoic acid, lysophosphatidic acid, skin peel agent (e.g., phenol, resorcinol and the like), vitamin B3 compound (e.g., niacinamide, nicotinic acid and nicotinic acid salts and ester, including non-vasodilating ester of nicotinic acid (such as tocopheryl nicotinate), nicotinyl amino acid, nicotinyl alcohol ester of carboxylic acid, nicotinic acid N-oxide and niacinamide N-oxide), vitamin B5, retinoid (e.g., retinol, retinal, retinoic acid, retinyl acetate, retinyl palmitate, retinyl ascorbate), and combinations thereof.

In some embodiments, the silk personal care composition optionally comprise a particle, wherein the particle may include polymeric particle, mica, silica, mud, and clay.

In some embodiments, the silk personal care composition comprises lyophilize silk powders derived from the silk solutions described above. In some embodiments, the silk personal care composition comprises lyophilize silk powders derived from the silk solutions, the silk fibroin protein fragments described above, and silk fibroin peptides having 2-50 amino acids derived from the hydrolysis of the silk fibroin protein and/or silk amino acids derived from the hydrolysis of the silk fibroin protein.

In some embodiments, the silk personal care composition comprises a polymeric particle formed of a polymer selected from the group consisting of an anionic and/or nonionic and/or zwitterion polymer. In some embodiments, the silk personal care composition comprises a polymeric particle formed of a polymer selected from the group consisting of polystyrene, polyvinylacetate, polydivinylbenzene, polymethylmethacrylate, poly-n-butylacrylate, poly-n-butylmethacrylate, poly-2-ethylhexylmethyacrylate, 6,12-nylon, poyurethanes, epoxy resins, styrene/vinyl acetate copolymers, styrene/trimethylaminoethyl methacrylate chloride copolymers, and combinations thereof.

In some embodiments, the silk personal care composition comprises a cationically charged polymeric particle formed of a hydrophobic polymer selected from the group consisting of polyethylene homopolymers, ethylene-acrylic acid copolymer, polyamide polymer having a molecular weight in the range of from about 6,000 Da to about 12,000 Da, polyethylene-vinyl acetate copolymer, silicone-synthetic wax copolymer, silicone-natural wax copolymer, candelilla-silicone copolymer, ozokerite-silicone copolymer, synthetic paraffin wax-silicone copolymer, and combinations thereof.

In some embodiments, the silk personal care composition comprises swollen polymer particles. In some embodiments, the swollen polymer particles are selected from the group consisting of particulate silicone polymers and surface-alkylated spherical silicon particles. In some embodiments, the silicone polymers forming the swollen polymer particles are selected from the group consisting of polydiorganosiloxanes, polymonoorganosiloxanes, and cross-linked polydimethyl siloxanes, crosslinked polymonomethyl siloxanes optionally having end groups including hydroxyl or methyl, and crosslinked polydimethyl siloxane (DC 2-9040 silicone fluid by Dow Corning). The polydisorganosiloxanes are preferably derived from suitable combinations of R₃SiO_{0.5} repeating units and R₂SiO repeating units. The polymonoorganosiloxanes are derived from R₁SiO_{1.5}. Each R independently represents an alkyl, alkenyl (e.g. vinyl), alkaryl, aralkyl, or aryl (e.g. phenyl) group. In some embodiments, R is a methyl group.

In some embodiments, the polymeric particles are nanoparticles having a median particle size less than 1000 nm. In some embodiments, the polymeric particles have a median particle size of about 5 nm to about 600 nm. In some embodiments, the polymeric particles have a median particle size of about 10 nm to about 500 nm. In some embodiments, the polymeric particles have a median particle size of about 10 nm to about 400 nm. In some embodiments, the polymeric particles have a median particle size of about 20 nm to about 300 nm. In some embodiments, the polymeric particles have a median particle size of about 50 nm to about 600 nm.

In some embodiments, the silk personal care composition comprises clay particles forming a dispersion or a suspension in the dermatologically acceptable carrier as disclosed herein. Throughout this specification, the term “clay” is intended to mean fine-grained earthy materials that become plastic when mixed with water. The clay may be a natural, synthetic or chemically modified clay. Clays include hydrous aluminum silicates that contain impurities, e.g. potassium, sodium, magnesium, or iron in small amounts.

In one embodiment, the clay is a material containing from 38.8% to 98.2% of SiO₂ and from 0.3% to 38.0% of Al₂O₃, and further contains one or more of metal oxides selected from Fe₂O₃, CaO, MgO, TiO₂, ZrO₂, Na₂O and K₂O. In some embodiments, the clay has a layered structure comprising hydrous sheets of octahedrally coordinated aluminium, magnesium or iron, or of tetrahedrally coordinated silicon.

In one embodiment, the clay is selected from the group consisting of kaolin, talc, 2:1 phyllosilicates, 1:1 phyllosilicates, smectite, bentonite, montmorillonites (also known as bentonites), hectorites, volchonskoites, nontronites, saponites, beidelites, sauconites, and mixtures thereof. In one embodiment, the clay is kaolin or bentonite. In some embodiments, the clay is a synthetic hectorite. In another embodiment, the clay is a bentonite.

In some embodiments, the clays have a cation exchange capacity of from about 0.7 meq/100 g to about 150 meq/100 g. In some embodiments, the clays have a cation exchange capacity of from about 30 meq/100 g to about 100 meq/100 g.

In some embodiments, the silk personal care composition optionally comprise a composite particle having an anionically charged clay electrostatically complexed with the cationically charged skin conditioning agents as disclosed herein.

Commercially available synthetic hectorites include those products sold under the trade names Laponite® RD, Laponite® RDS, Laponite® XLG, Laponite® XLS, Laponite® D, Laponite® DF, Laponite® DS, Laponite® S, and Laponite® JS (Southern Clay products, Texas, USA). Commercially available bentonites include those products sold under the trade names Gelwhite® GP, Gelwhite® H, Gelwhite® L, Mineral Colloid® BP, Mineral Colloid® MO, Gelwhite® MAS 100 (sc), Gelwhite® MAS 101, Gelwhite® MAS 102, Gelwhite® MAS 103, Bentolite® WH, Bentolite® L10, Bentolite® H, Bentolite® L, Permont® SX10A, Permont® SC20, and Permont® HN24 (Southern Clay Products, Texas, USA); Bentone® EW and Bentone® MA (Dow Corning); and Bentonite® USP BL 670 and Bentolite® H4430 (Whitaker, Clarke & Daniels).

In order to achieve good deposition onto skin and a stable formulation, the particles have a median particle size ranging from about 1 μm to about 10.0 μm. In some embodiments, the particles have a median particle size ranging from about 4 μm to about 10 μm. In some embodiments, the particles have a median particle size selected from: about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.1 μm, about 2.2 μm, about 2.3 μm, about 2.4 μm, about 2.5 μm, about 2.6 μm, about 2.7 μm, about 2.8 μm, about 2.9 μm, about 3.0 μm, about 3.1 μm, about 3.2 μm, about 3.3 μm, about 3.4 μm, about 3.5 μm, about 3.6 μm, about 3.7 μm, about 3.8 μm, about 3.9 μm, about 4.0 μm, about 4.1 μm, about 4.2 μm, about 4.3 μm, about 4.4 μm, about 4.5 μm, about 4.6 μm, about 4.7 μm, about 4.8 μm, about 4.9 μm, about 5.0 μm, about 5.1 μm, about 5.2 μm, about 5.3 μm, about 5.4 μm, about 5.5 μm, about 5.6 μm, about 5.7 μm, about 5.8 μm, about 5.9 μm, about 6.0 μm, about 6.1 μm, about 6.2 μm, about 6.3 μm, about 6.4 μm, about 6.5 μm, about 6.6 μm, about 6.7 μm, about 6.8 μm, about 6.9 μm, about 7.0 μm, about 7.1 μm, about 7.2 μm, about 7.3 μm, about 7.4 μm, about 7.5 μm, about 7.6 μm, about 7.7 μm, about 7.8 μm, about 7.9 μm, about 8.0 μm, about 8.1 μm, about 8.2 μm, about 8.3 μm, about 8.4 μm, about 8.5 μm, about 8.6 μm, about 8.7 μm, about 8.8 μm, about 8.9 μm, about 9.0 μm, about 9.1 μm, about 9.2 μm, about 9.3 μm, about 9.4 μm, about 9.5 μm, about 9.6 μm, about 9.7 μm, about 9.8 μm, about 9.9 μm, and about 10.0 μm.

In some embodiments, the weight ratio of the cationically charged skin-conditioning agent to the clay is from 0.05:1 to 20:1. In some embodiments, the weight ratio of the cationically charged skin-conditioning agent to the clay is from 0.1:1 to 10:1. In some embodiments, the weight ratio of the cationically charged skin-conditioning agent to the clay is from 0.2:1 to 5:1. In some embodiments, the weight ratio of the cationically charged skin conditioning agent to the clay is selected from 0.05:1, 0.1:1, 0.2:1, 0.5:1, 0.75:1, 1:1, 1.5:1, 2:1, 2.5:1, 3:1, 3.5:1, 4.0:1, 4.5:1, 5.0:1, 5.5:1, 6.0:1, 6.5:1, 7.0:1, 7.5:1, 8.0:1, 8.5:1, 9.0:1, 9.5:1, 10.0:1, 10.5:1, 11.0:1, 11.5:1, 12.0:1, 12.5:1, 13.0:1, 13.5:1, 14.0:1, 14.5:1, 15.0:1, 15.5:1, 16.0:1, 16.5:1, 17.0:1, 17.5:1, 18.0:1, 18.5:1, 19.0:1, 19.5:1, and 20.0:1.

In some embodiments, the silk personal care composition comprises about 0.01 wt. to about 10.0 wt. % of the particles. In some embodiments, the silk personal care composition comprises about 0.1 wt. % to about 10.0 wt. % of the particles. In some embodiments, the silk personal care composition comprises about 0.1 wt. % to about 2.0 wt. % of the particles. In some embodiments, the silk personal care composition comprises about 1.0 wt. % to about 9.0 wt. % of the particles. In some embodiments, the silk personal care composition comprises about 1.0 wt. % to about 5.0 wt. % of the particles. In some embodiments, the amount of particle in the silk personal care composition is selected from the group consisting of about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7.0 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8.0 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9.0 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, and about 10.0 wt. % by the total weight of the silk personal care composition.

In some embodiments, the silk personal care composition optionally comprise a colloidal stabilizer to maintain particle dispersive stability, particularly of larger sized particles. Suitable colloidal stabilizer is selected from the group consisting of propylene oxide-ethylene oxide copolymers or ethyleneoxide-propylenoxide grafted polyethylenimines, polyoxyethylene (20-80 units POE) isooctylphenyl ether, fatty alcohol ethoxylates, polyethoxylated polyterephthalate block co-polymers containing polyvinylpyrrolidone, copolymers containing vinylpyrolidone repeating units, and combinations thereof.

In some embodiments, the silk personal care composition optionally comprises therapeutic agent selected from the group consisting of vasoconstrictor, anti-histamine, naphazoline hydrochloride, ephedrine hydrochloride, phenylephrine hydrochloride, tetrahydrozoline hydrochloride, pheniramine maleate, and combinations thereof.

In some embodiments, the silk personal care composition optionally comprises a coloring agent selected from the group consisting of natural pigments and dyes, synthetic pigments and dyes, lakes, and combination thereof.

In some embodiments, the silk personal care composition optionally comprises pigments and dyes selected from the group consisting of grape skin pigment, carmine dye, pigment orange rouge, anthocyanins, carminic acid, betacyanins, amaranthin, flavonoids, Verbena hybrida haematochrome, berberine-based pigment, hinokitiol, betel nut pigment, quercetin, rutin, logwood pigment, henna tannin and catechin, curcumin, cactus flavin, rosewood pigment, bixin or decreasing annatto, saffron extract, buckwheat extract, crocin, genipin, henna (Lawsonia alba), camomile (Matricaria chamomila or Anthemis nobilis), indigo, gardenia pigment, gardenia red, pigment, gardenia enzyme-treated pigment, lac pigment, cochineal pigment, brazilin pigment, annatto pigment, turmeric pigment, logwood pigment, and walnut hull extract, and combination thereof. In some embodiments, the silk personal care composition optionally comprises synthetic pigments and dyes, lakes selected from the group consisting of D&C pigment, FD&C pigment, HC Blue 2, HC Yellow 4, HC Red 3, Disperse Violet 4, Disperse Black 9, HC Blue 7, HC Yellow 2, Disperse Blue 3, Disperse violet 1, Citrus Red No. 2 (CAS No. 6358-53-8), FD&C Yellow No. 6 (CAS No. 2783-94-0), FD&C Yellow No. 6 Lakes (CAS No. 15790-07-5), FD&C Red No. 40 (CAS No. 25956-17-6), FD&C Red No. 40 Lakes (CAS No. 68583-95-9), FD&C Yellow No. 5 (CAS No. 1934-21-0), FD&C Yellow No. 5 Lakes (CAS No. 12225-21-7), Acid Red 18 (CAS No. 2611-82-7), Orange B (CAS No. 15139-76-1), FD&C Green No. 3 (CAS No. 2352-45-9), FD&C Blue No. 1 (CAS No. 3844-45-9), FD&C Blue No. 1 Lakes (CAS No. 68921-42-6), FD&C Red No. 3 (CAS No. 16423-68-0), FD&C Red No. 3 Lakes (CAS No. 12227-78-0), FD&C Blue No. 2 (CAS No. 860-22-0), FD&C Blue No. 2 Aluminum Lake (CAS No. 16521-38-3), Arianor dyes basic brown 17, C.I. (color index) no. 12251; basic red 76, CI.12245; basic brown 16, CI.12250; basic yellow 57, CI.12719 and basic blue 99, CI.56059 and further direct action dyes such as acid yellow 1, C.I.10316 (D&C yellow No. 7); acid yellow 9, C.I.13015; basic violet C.I.45170; disperse yellow 3, C.I.11855; basic yellow 57, CI.12719; disperse yellow 1, CI.10345; basic violet 1, CI.42535, basic violet 3, C.I. 42,555; greenish blue, C.I. 42090 (FD&C Blue No. 1); yellowish red, C.I. 14700 (FD&C red No. 4); yellow, CI.19140 (FD&C yellow No. 5); yellowish orange, CI.15985 (FD&C yellow No. 6); bluish green, C.I.42053 (FD&C green No. 3); yellowish red, CI.16035 (FD&C red No. 40); bluish green, CI.61570 (D&C green No. 3); orange, C.I.45370 (D&C orange No. 5); red, CI.15850 (D&C red No. 6); bluish red, CI.15850 (D&C red No. 7); slight bluish red, CI.45380 (D&C red No. 22); bluish red, CI.45410 (D&C red No. 28); bluish red, CI.73360 (D&C red No. 30); reddish purple, CI.17200 (D&C red No. 33); dirty blue red, CI.15880 (D&C red No. 34); bright yellow red, CI.12085 (D&C red No. 36); bright orange, CI.15510 (D&C orange No. 4); greenish yellow, CI.47005 (D&C yellow No. 10); bluish green, CI.59040 (D&C green No. 8); bluish violet, CI.60730 (Ext. D&C violet No. 2); greenish yellow, CI.10316 (Ext. D&C yellow No. 7), Acridine Orange C.I.46005, titanian oxide, iron oxide, zirconian oxide, carbon black, phthalocyanine pigment, quinacridone pigment, azo pigment, xanthene pigment, nitroaryl amine, aminoanthraquinone, anthraquinone dye, naphthoquinone dye, metal oxide coated mica, and combination thereof.

In some embodiments, the silk personal care composition contains a single coloring agent at about 0.001 wt. % to about 6.0 wt. % by the total weight of the silk personal care composition. In some embodiments, the silk personal care composition contains each coloring agent at about 0.01 wt. % to about 2.0 wt. % by the total weight of the silk personal care composition.

In some embodiments, the silk personal care composition contains a coloring agent blend at about 0.01 wt. % to about 15.0 wt. % by the total weight of the silk personal care composition. In some embodiments, the silk personal care composition contains a coloring agent blend at about 0.1 wt. % to about 10.0 wt. % by the total weight of the silk personal care composition. In some embodiments, the silk personal care composition contains a coloring agent blend at about 0.5 wt. % to about 5.0 wt. % by the total weight of the silk personal care composition.

In some embodiments, the silk personal care composition further comprises a preservative to protect against the growth of potentially harmful microorganisms. While it is in the aqueous phase that microorganisms tend to grow, microorganisms can also reside in the anhydrous or oil phase. As such, preservatives, which have solubility in both water and oil are employed. In some embodiments, the preservative is selected from the group consisting of alkyl esters of parahydroxybenzoic acid, hydantoin derivative, propionate salt, quaternary ammonium compound, and combinations thereof.

In some embodiments, the preservative is selected from the group consisting of methylparaben, imidazolidinyl urea, sodium dehydroacetate, propylparaben, trisodium ethylenediamine tetraacetate (EDTA), benzyl alcohol, and combinations thereof. The preservative can be selected to avoid possible incompatibilities between the preservative and other ingredients. In some embodiments, the silk personal care comprises about 0.01 wt. % to about 2.0 wt. % of the preservatives.

In some embodiments, the silk personal care composition further comprises a film-forming agent to form a protective film on the skin that protects the skin from damages caused by various environmental factors such as losing moisture, washing, cleansing application, and ultraviolet light.

In some embodiments, the silk fibroin protein fragments may be functioning as film forming agent for the personal care compositions. Silk fibroin protein fragments have protein structure similar to the skin. The silk fibroin protein fragments having a weight average molecular weight selected from between about 1 kDa to about 144 kDa easily form resilient and transparent film on the skin. The silk fibroin protein fragments are ideally suited for film-forming and coating applications due to their ability to self-assemble in solution. The self-assembly property of silk fibroin protein fragments is due to the formation of anti-parallel beta-pleated sheets via hydrogen bonding and electrostatic interactions.

In some embodiments, the silk fibroin protein fragment has a weight average molecular weight selected from between 1 kDa to 19 kDa, from between 1 kDa to 10 kDa, from between 1 kDa to 5 kDa, from between 5 kDa to 10 kDa, and from between 5 kDa to 20 kDa, and form a durable and wear-resistant silk film coating on the skin. In some embodiments, the silk fibroin protein fragment has a weight average molecular weight selected from between 10 kDa to 20 kDa, and form a durable and wear-resistant silk film coating on the skin.

In some embodiments, the silk fibroin protein fragment has a weight average molecular weight selected from between 20 kDa to 144 kDa, and form a durable and wear-resistant silk film coating on the skin. In some embodiments, the silk fibroin protein fragment has a weight average molecular weight selected from between 20 kDa to 90 kDa, from between 20 kDa to 39 kDa, and from between 39 kDa to 90 kDa, and form a durable and wear-resistant silk film coating on the skin.

In some embodiments, the silk fibroin protein fragments forms soft-holding strength films on the skin is used in an amount ranges from about 1.0 wt. % to about 3.0 wt. % by the total weight of the personal care composition. In some embodiments, the weight amount of the silk fibroin protein fragments in the silk personal care composition is selected from the group consisting of about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, and about 3.0% (w/w).

In some embodiments, additional film forming agent is added to the silk personal care composition. In some embodiments, the additional film forming agent is selected from the group consisting of polyacrylic acids, polyacrylates, polyacylamides, silicones, polyquaternium compounds, elastomeric materials, latexes, polyurethanes, polyethylenes, polystyrenes, nylon, polysaccharides, proteins, polysiloxanes (e.g. polyether modified silicone), long chain alkyl quaternary ammoniums, polyvinylpyrrolidone (PVP), PVPK30, quaternary ammonium derivatives of cellulose ethers, copolymers of hydroxyethylcellulose and dimethyldiallylammonium halide, quaternary ammonium derivatives of copolymers of vinylpyrrolidone and dimethylaminoethylmethacrylate, copolymers of acrylamide and dimethyldiallylammonium halide, quaternary ammonium derivatives of copolymers of acrylamide and dimethylaminoethylmethacrylate, shellac, polyvinylpyrrolidone-ethyl methacrylate-methacrylic acid terpolymer, vinyl acetate-crotonic acid copolymer, vinyl acetate-crotonic acid-vinyl neodeconate terpolymer, poly(vinylpyrrolidone)-ethyl methacrylate methacrylic acid copolymer, vinyl methyl ether-maleic anhydride copolymer, octylacrylamide-acrylate-butylaminoethyl-methacrylate copolymer, and poly(vinylpyrrolidone-dimethylaminoethylmethacrylate) copolymer and derivatives, polyquaternium-46, chitosan, microcrystalline chitosan, quaternary ammonium derivative of chitosan, quaternary cellulose derivatives; vinylpyrrolidone-vinyl acetate copolymers, polyvinylpyrrolidone-ethyl methacrylate-methacrylic acid terpolymer, vinyl acetate-crotonic acid copolymer, vinyl acetate-crotonic acid-vinyl neodeconate terpolymer, poly(vinylpyrrolidone)-ethyl methacrylate methacrylic acid copolymer, vinyl methyl ether-maleic anhydride copolymer, octylacrylamide-acrylate-butylaminoethyl-methacrylate copolymer, and poly(vinylpyrrolidone-dimethylaminoethyl-methacrylate) copolymer, shellac, collagen, keratin, and elastin.

In some embodiments, the additional film-forming agent is selected from the group consisting of silk fibroin protein fragments, PVP-PVP-VA, polyquaternium-46, sucrose acetate isobutyrate, high molecular weight polybutenes e.g. polybutene, and combination thereof. In some embodiments, the additional film-forming agent comprises silk fibroin protein fragments and polyquaternium-46. In some embodiments, the additional film-forming agent is selected from the group consisting of copolymers of acrylamide and dimethyldiallylammonium halide, copolymers of hydroxyethylcellulose and dimethyldiallylammonium halide, and combination thereof.

The silk personal care compositions containing filming-forming polymers having quaternary ammonium groups provide more effective and more durable films when applied to skin. In some embodiments, the additional film forming agent comprises amino-modified silicone resin selected from the group consisting of polydimethylsiloxane containing aminoethylaminopropyl, N-(aminoethylaminomethyl) phenyl, N-(2-aminoethyl)-3-aminopropyl, and bis(2-hydroxyethyl)-3-aminopropyl groups), DC 929 grade silicone fluid (Dow Corning), and combination thereof.

In some embodiments, the film-forming agent is selected from the group consisting of hydroxyethylcellulose, hydroxypropylcellulose, hydroxypropyl methylcellulose, ethylhydroxyethyl cellulose, and combinations thereof.

In some embodiments, the silk personal care composition comprises about 1.0 wt. % to about 30.0 wt. % of the additional film-forming agent. In some embodiments, the silk personal care composition comprises about 1.5 wt. % to about 10.0 wt. % of the additional film-forming agent.

### (4) Soap Bar

Personal cleansing compositions are available in various forms including soap bar, liquid soaps, creams and gels. Traditional soap bars are composed of the alkali metal salts of fatty acids made by saponification of natural fats with alkali metal bases. Soap bar compositions generally contain from about 40 wt. % to about 76 wt. % total fatty matter (TFM). Soaps having TFM in the range of 60% to 76% are called “toilet soaps”. Soap bars having TFM in the range of 40% to 60% are called “bathing bars”.

Due to the basic pH value, conventional soap bar often leaves the skin dry, with rough texture, defatted and irritated. Therefore, there is a need to provide a soap bars that not only effectively cleanse the skin, mild to skin, but also impart skin caring benefits, for example, the soap bars effectively deliver skin caring active agent such as moisturizer, humectant to the skin of the user during wash.

In some embodiments, the silk cleansing composition is formulated as a soap bar comprising (1) the soap base as described above, (2) about 1.0 wt. % to about 5.0 wt. % of lyophilized silk fibroin protein powder derived from the silk solution described above; (3) about 2.0 wt. % to about 6.0 wt. % of silk fibroin protein fragments as described above, (4) about 0.5 wt. % to about 6.0 wt. % of a mixture of the amino acids serine, alanine and glycine derived from the hydrolysis of silk fibroin protein.

Silk powder gives relief from sunburns due to its crystalline structure capable of deflecting UV radiation and as demulcent providing a buffer between human skin and the environment.

In some embodiments, the soap bar is substantially free of synthetic surfactant. The term “substantially free” as used herein refers to a level of synthetic surfactant in the soap bar is less than 0.8 wt. % by the total weight of the soap bar. In some embodiments, the level of synthetic surfactant in the soap bar is less than 0.5 wt. % by the total weight of the soap bar. In some embodiments, the level of synthetic surfactant in the soap bar is less than 0.3 wt. % by the total weight of the soap bar. In some embodiments, the level of synthetic surfactant in the soap bar is less than 0.1 wt. % by the total weight of the soap bar. In some embodiments, the level of synthetic surfactant in the soap bar is 0 wt. % by the total weight of the soap bar.

In some embodiments, the soap bar further comprises about 0.5 wt. % to about 3.0 wt. % of an anticracking agent selected from the group consisting of carboxymethylcellulose, polyacrylate polymer, and mixtures thereof.

In some embodiments, the soap bar comprises (1) 35 wt. % to 65 wt. % soap as described above, (2) about 1.0 wt. % to about 5.0 wt. % of lyophilized silk fibroin protein powder derived from the silk solutions as described above; (3) about 2.0 wt. % to about 6.0 wt. % of silk fibroin protein fragments as described above, (4) about 0.5 wt. % to about 6.0 wt. % of a mixture of the amino acids serine, alanine and glycine derived from the hydrolysis of silk fibroin protein, (5) 5.0 wt. % to 15.0 wt. % of water, (6) 0 wt. % to 2 wt. % of polyols, (7) 0.25 wt. % to 0.75 wt. % of chelating agent, (8) 0.5 wt. % to 3.0 wt. % of emollient, (9) 0.1 wt. % to 5.0 wt. % of skin care active agent, (10) 0.01 wt. % to 1.0 wt. % of colorant, and (11) 1.0 wt. % to 30 wt. % of wax.

In some embodiments, the soap bars can contain water. In some embodiments, the amount of water contained in the soap bar is selected from the group consisting of 0 wt. % to about 20.0 wt. %, about 15.0 wt. % or less, about 10.0 wt. % or less, about 5.0 wt. % to about 20 wt. %, about 5.0 wt. % to about 15.0 wt. %, about 10.0 wt. % to about 20.0 wt. %, and about 10.0 wt. % to about 15.0 wt. % by the total weight of the soap bar.

Optionally, the cleansing bar can contain foaming agent. In some embodiments, the foaming agent is selected from the group consisting of amphoteric surfactant, cocomonoethanolamide (CMEA), cocoamidopropylamine oxide, cetyl dimethylamine chloride, decylamine oxide, lauryl/myristyl amidopropyl amine oxide, lauramine oxide, alkyldimethyl amine n-oxide, myristamine oxide, and combinations thereof.

In some embodiments, the foaming agent is present in the soap bar at an amount ranging from about 2.0 wt. % to about 10.0 wt. % by the total weight of the soap bar.

### II. Silk Oral Care Products

To be able to sufficiently guarantee the capability of chewing, e.g. foods, during a whole lifetime it is necessary to keep the teeth in a good condition and to obtain a good oral hygiene.

To obtain and maintain a good dental and oral hygiene, the teeth must at least be brushed once every day using toothpaste or the like. Further, the mouth should regularly be rinsed with a mouthwash.

The brushing of the teeth primarily helps removing food particles from the teeth, and constitutes an important first step in preventing dental caries, which may cause dental holes, and further in preventing dental diseases.

A good dental hygiene is necessary to prevent the formation of plaque and tartar, outbreak of oral diseases, and adherence of stains from tea, coffee and tobacco smoking that removes the basis for obtaining a good appearance when e.g. smiling. Furthermore, teeth brushing is useful to eliminate the unpleasant incidences of bad breath, or e.g. an occasional garlic smelling breath.

In general, it is believed that dental caries arise when cariogenic microorganisms, such as Streptococcus mutans or Streptococcus sanguis, grow in oral cavities. Sucrose, derived from foods, can be converted into water-soluble and insoluble polysaccharides by glucosyltransferase (GTF) produced by cariogenic microorganisms. These polysaccharides coat the surface of the cariogenic microorganisms and other bacteria, and finally adhere onto the tooth surface to form a dental plaque. Bacteria contained in the dental plaque can degrade the polysaccharides to acids, such as lactic acid that can erode the tooth enamel leading to dental holes. As plaque continues to accumulate, rock hard white or yellowish deposits may arise. These deposits are called calcified plaque, calculus or tartar, and are formed in the saliva from plaque and minerals, such as calcium. Accumulation of tartar below the gum line may cause periodontal disease.

For an effective ingredient of an oral care composition to have a beneficial or therapeutic effect, whether for oral cleaning, treatment, or tooth whitening, the effective ingredient must reach and preferably maintain effective contact with the oral care feature long enough to provide its intended effect.

Oral care compositions such as mouthwashes and toothpastes are generally designed to inhibit or kill microorganisms that cause gum disease, retard or stop plaque formation, prevent caries and to provide teeth whitening. Teeth whitening is typically done through the use of abrasive agents or bleaching agents. Factors that cause teeth staining include the use of coffee, tea, red wine, cola, tobacco products, or other stain promoting oral products. The disadvantage of using highly abrasive toothpastes, typically used in whitening toothpaste formulations, is the potential for the destruction of tooth enamel. The conventional tooth bleaching agents, such as hydrogen peroxide, can be harsh to oral tissue and can often cause tooth sensitivity.

In some embodiments, this disclosure provides an oral care composition comprising the silk fibroin protein fragments and the orally acceptable carrier as described above. In some embodiments, the oral care composition further comprises an additive selected from the group consisting of a filler, a diluent, a remineralizing agent, an anti-calculus agent, an anti-plaque agent, a buffer, an abrasive, an alkali metal bicarbonate salt, a binder, a thickening agent, a humectant, a whitening agent, a bleaching agent, a stain removing agent, a surfactant, titanium dioxide, a flavoring agent, xylitol, a coloring agent, a foaming agent, a sweetener, an antibacterial agent, a preservative, a vitamin, a pH-adjusting agent, an anti-caries agent, a teeth whitening active agent, a desensitizing agent, a coolant, a salivating agent, a warming agent, a numbing agent, a chelating agent, and combinations thereof.

In some embodiments, the oral care composition is formulated as a product selected from the group consisting of a toothpaste, a dentifrice, a tooth powder, an oral gel, an aqueous gel, a non-aqueous gel, a mouth rinse, a mouth spray, a plaque removing liquid, a denture product, a dental solution, a lozenge, an oral tablet, a chewing gum, a candy, a fast-dissolving film, a strip, a dental floss, a tooth glossing product, a finishing product, and an impregnated dental implement.

In some embodiments, the oral care composition is formulated as a toothpaste comprising a tooth care active agent selected from the group consisting an abrasive, lyophilized silk powder, an anti-calculus agent, an anti-plaque agent, a humectant, a whitening agent, an anti-caries agent, a desensitizing agent, a coolant, a salivating agent, a warming agent, a numbing agent, and combinations thereof.

In some embodiments, the oral care composition is formulated as a tooth remineralization composition comprising a therapeutically effective amount of a remineralizing agent. In some embodiments, the remineralizing agent is selected from the group consisting of fluoride, calcium and/or phosphate, amorphous calcium phosphate (ACP), tricalcium phosphate, casein phosphoprotein-ACP, bioactive glass, calcium sodium phosphosilicate, arginine bicarbonate-calcium carbonate complex. In some embodiments, the tooth remineralization composition is formulated as a remineralizing gel, a remineralizing mouthwash, a remineralizing tooth powder, a remineralizing chewing gum, a remineralizing lozenge, or a remineralizing toothpaste.

In some embodiments, the oral care composition comprises one or more tooth care active agent selected from the group consisting of abrasives, anti-calculus agents, remineralizing agents, antiplaque agents, anti-caries agents, teeth whitening agents, tartar control agents, desensitizing agents, coolants, salivating agents, warming agents, and numbing agents, tooth remineralization agents, fluoride ion source compounds, calcium ion source compounds, phosphate ion source compounds, binders, thickening agents, humectants, bleaching agents, breath freshening agent, stain removing agents, surfactants, titanium dioxide, flavoring agents, coloring agents, foaming agents, sweeteners, antibacterial agents, a preservatives, vitamins, chelating agents, and combinations thereof.

In some embodiments, the abrasive is selected to be compatible within the oral care composition and does not excessively abrade dentin. In some embodiments, the abrasive is selected from the group consisting of silica, insoluble sodium polymetaphosphate, hydrated alumina, calcium carbonate, dicalcium orthophosphate dihydrate, calcium pyrophosphate, tricalcium phosphate, calcium polymetaphosphate, perlite, expanded perlite, bioglasses, resinous abrasive materials such as particulate condensation products of urea and formaldehyde, and combinations thereof. In some embodiments, the abrasives are thermo-setting polymerized resins selected from the group consisting of melamine, phenolic, urea, melamine-urea, melamine-formaldehyde, urea-formaldehyde, melamine-urea-formaldehydes, cross-linked epoxide, cross-linked polyester, and combinations thereof. Silica dental abrasives are preferred because of their unique benefits of exceptional dental cleaning and polishing performance without unduly abrading tooth enamel or dentine. The silica abrasive polishing materials have an average particle size ranging between about 0.1 to about 30 microns, and preferably from about 5 to about 15 microns. The abrasive can be precipitated silica or silica gels.

In some embodiments, the oral care composition comprises about 6.0 wt. % to about 70.0 wt. % of the abrasive. In some embodiments, the oral care composition comprises about 10 wt. % to about 50 wt. % of the abrasive.

In some embodiments, the oral care composition further comprises a humectant to prevent loss of water from the oral care composition. Humectant serves to keep oral care compositions from hardening upon exposure to air, to give oral care compositions a moist feel to the mouth, and, for particular humectants, to impart desirable sweetness of flavor to oral care compositions. In some embodiments, the humectant is selected from the group consisting of glycerol, polyol, sorbitol, polyethylene glycols (PEG), propylene glycol, 1,3-propanediol, 1,4-butanediol, hydrogenated partially hydrolyzed polysaccharides, butylene glycol, and combinations thereof. In some embodiments, the humectant is sorbitol and/or glycerin.

In some embodiments, the oral care composition comprises 0% to about 80.0 wt. % of the humectant. In some embodiments, the oral care composition comprises about 5.0 wt. % to about 70 wt. % of the humectant. In some embodiments, the oral care composition comprises 0% to about 70 wt. % of the humectant. In some embodiments, the oral care composition comprises about 20.0 wt. % to about 50.0 wt. % of the humectant.

In some embodiments, the oral care composition further comprises a binder/thickener selected from the group consisting of silica, starch, tragacanth gum, xanthan gum, extracts of Irish moss, alginates, pectin, cellulose derivatives, such as hydroxyethyl cellulose, sodium carboxymethyl cellulose and hydroxypropyl cellulose, polyacrylic acid and its salts, polyvinylpyrrolidone, and combinations thereof. The binder/thickener helps stabilizing the toothpaste.

In some embodiments, the oral care composition comprises about 0.1 wt. % to about 20.0 wt. % of the binder/thickener. In some embodiments, the oral care composition comprises about 0.1 wt. % to about 10.0 wt. % of the binder/thickener.

In some embodiments, the oral care composition further comprises a coolant. In some embodiments, the coolant is selected from the group consisting of carboxamide, menthol, ketal, diol, 3-1-menthoxypropane-1,2-diol (TK-10™ by Takasago), menthone glycerol acetal (MGA™ by Haarmann and Reimer), menthyl lactate (Frescolat™ by Haarmann and Reimer), and combinations thereof. In some embodiments, the coolant is N-ethyl-p-menthan-3-carboxamide (WS-3™), N-2,3-trimethyl-2-isopropylbutanamide (WS-23™), and combinations thereof. The terms menthol and menthyl as used herein include dextro- and levorotatory isomers of these compounds and racemic mixtures thereof.

In some embodiments, the oral care composition further comprises a desensitizing agents. In some embodiments, the desensitizing agent is selected from the group consisting of potassium salt, capsaicin, eugenol, a strontium salt, and combinations thereof.

In some embodiments, the oral care composition further comprises a teeth-whitening agent. In some embodiments, the teeth whitening agent is selected from the group consisting of peroxides, metal chlorites, perborates, percarbonates, peroxyacids, persulfates, hypochlorite, chlorine dioxide, and combinations thereof. In some embodiments, the peroxide compound is selected from the group consisting of hydrogen peroxide, urea peroxide, calcium peroxide, and combinations thereof. In some embodiments, the metal chlorites is selected from the group consisting of calcium chlorite, barium chlorite, magnesium chlorite, lithium chlorite, sodium chlorite, and potassium chlorite. In some embodiments, the metal chlorite is sodium chlorite. In some embodiments, the percarbonate is sodium percarbonate. In some embodiments, the whitening agent is selected from the group consisting of potassium, ammonium, sodium and lithium persulfates; potassium, ammonium, sodium and lithium perborate mono- and tetrahydrates; sodium pyrophosphate peroxyhydrate; and combinations thereof.

In some embodiments, the oral care composition further comprises an anticalculus agent. In some embodiments, the anticalculus agent is a pyrophosphate salt to provide pyrophosphate ions. In some embodiments, the pyrophosphate salt is selected from the group consisting of dialkali metal pyrophosphate salts, tetraalkali metal pyrophosphate salts, disodium dihydrogen pyrophosphate (Na₂H₂P₂O₇), tetrasodium pyrophosphate (Na₄P₂O₇), anhydrous tetrapotassium pyrophosphate (K₄P₂O₇), hydrated K₄P₂O₇, and combinations thereof.

In some embodiments, the oral care composition further comprises a chelating agent selected from the group consisting of tartaric acid, alkali metal tartarate, citric acid, alkali metal citrates, and combinations thereof. Chelating agents are able to complex calcium found in the cell walls of the bacteria. In some embodiments, the chelating agent is selected from the group consisting of disodium tartrate, dipotassium tartrate, sodium potassium tartrate, sodium hydrogen tartrate, potassium hydrogen tartrate, and combinations thereof. In some embodiments, the chelating agent is sodium or potassium citrate. In some embodiments, the chelating agent is sodium citrate. In some embodiments, the chelating agent is a citric acid/alkali metal citrate combination. In some embodiments, the chelating agent is alkali metal salts of tartaric acid. In some embodiments, the chelating agent is sodium tartrate or potassium tartrate.

In some embodiments, the oral care composition comprises about 0.1 wt. % to about 2.5 wt. % of the chelating agent. In some embodiments, the oral care composition comprises about 0.5 wt. % to about 2.5 wt. % of the chelating agent. In some embodiments, the oral care composition comprises about 1.0 wt. % to about 2.5 wt. % of the chelating agent.

In some embodiments, the oral care composition further comprises a water-soluble fluoride compound in an amount sufficient to give a fluoride ion ranging from about 0.0025 wt. % to about 5.0 wt. %, or from about 0.005 wt. % to about 2.0 wt. % at 25° C. to provide anticaries effectiveness. In some embodiments, the fluoride ion sources compound is selected from the group consisting of stannous fluoride, sodium fluoride, potassium fluoride, sodium monofluorophosphate, and combinations thereof. In some embodiments, the fluoride ion sources compound is selected from the group consisting of stannous fluoride, sodium fluoride and combinations thereof.

In some embodiments, the oral care composition further comprises an anionic foaming surfactant that foams at a wide pH range from 4.5 to 9.0. In some embodiments, the anionic foaming surfactant can be one or more of anionic, nonionic, amphoteric, zwitterion, or cationic surfactant. In some embodiments, suitable anionic foaming surfactant is selected from the group consisting of water-soluble salts of alkyl sulfates with alkyl chain having 8 to 20 carbon atoms (e.g., sodium alkyl sulfate), water-soluble salts of sulfonated monoglycerides of fatty acids with alkyl chain having 8 to 20 carbon atoms (e.g., sodium lauryl sulfate, or sodium coconut monoglyceride sulfonate), sarcosinate, sodium lauroyl sarcosinate, taurate, sodium lauryl sulfoacetate, sodium lauroyl isethionate, sodium lauryl carboxylate, and sodium dodecyl benzenesulfonate, and combinations thereof. In some embodiments, the oral care composition comprises the anionic foaming surfactant selected from the group consisting of sarcosinate surfactant, isethionate surfactant, taurate surfactant, sodium or potassium lauroyl sarcosinate, sodium or potassium myristoyl sarcosinate, sodium or potassium palmitoyl sarcosinate, sodium or potassium stearyl sarcosinate, sodium or potassium oleoyl sarcosinate, and combinations thereof.

In some embodiments, the oral care composition comprises about 0.025 wt. % to about 9.0 wt. % of the anionic foaming surfactant. In some embodiments, the oral care composition comprises about 0.05 wt. % to about 5.0 wt. % of the anionic foaming surfactant. In some embodiments, the oral care composition comprises about 0.1 wt. % to about 2.5 wt. % of the anionic foaming surfactant. In some embodiments, the oral care composition comprises about 0.2 wt. % to about 3.0 wt. % of the anionic foaming surfactant. In some embodiments, the oral care composition comprises about 0.3 wt. % to about 2.5 wt. % of the anionic foaming surfactant. In some embodiments, the oral care composition comprises about 0.5 wt. % to about 2.0 wt. % of the anionic foaming surfactant.

In some embodiments, the oral care composition further comprises a cationic foaming surfactant selected from aliphatic quaternary ammonium compounds with one long alkyl chain having 8 to 18 carbon atoms. In some embodiments, the cationic foaming surfactant is selected from the group consisting of lauryl trimethylammonium chloride, cetyl pyridinium chloride, cetyl trimethylammonium bromide, di-isobutylphenoxyethyl-dimethylbenzylammonium chloride, coconut alkyltrimethylammonium nitrite, cetyl pyridinium fluoride, and combinations thereof.

In some embodiments, the oral care composition further comprises alkyl dimethyl betaines as surfactant. In some embodiments, the alkyl dimethyl betaine is selected from the group consisting of decyl betaine, 2-(N-decyl-N,N-dimethylammonio) acetate, coco betaine, myristyl betaine, palmityl betaine, lauryl betaine, cetyl betaine, cetyl betaine, stearyl betaine, and combinations thereof. In some embodiments, the amido-betaine is selected from the group consisting of cocoamidoethyl betaine, cocoamidopropyl betaine, lauramidopropyl betaine, and combinations thereof. In some embodiments, the betaines is cocoamidopropyl betaine. In some embodiments, the betaines is lauramidopropyl betaine.

In some embodiments, the oral care composition further comprises thickening agents in toothpaste or gels. In some embodiments, the thickening agent is selected from the group consisting of sodium carboxymethylcellulose, sodium carboxymethyl hydroxyethyl cellulose and, hydroxyethyl cellulose, carboxyvinyl polymers, carrageenan, laponite, gum karaya, xanthan gum, guar gum, gum arabic, gum tragacanth, colloidal magnesium aluminum silicate, and fumed silica.

In some embodiments, the oral care composition further comprises flavoring and sweetening agents. In some embodiments, the flavoring agent is selected from the group consisting of wintergreen oil, peppermint oil, spearmint oil, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal, and combinations thereof. In some embodiments, the oral care composition comprises about 0.001 wt. % to about 5.0 wt. % of the flavoring agent. In some embodiments, the oral care composition comprises about 0.001 wt. % to about 1.0 wt. % of the flavoring agent. In some embodiments, the oral care composition comprises about 0.1 wt. % to about 2.0 wt. % of the flavoring agent.

In some embodiments, the sweetening agent is selected from the group consisting of sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame, sucralose and cyclamate salts, especially sodium cyclamate and sodium saccharin, and combinations thereof. In some embodiments, the oral care composition comprises about 0.1 wt. % to about 10 wt. % of the sweetening agent. In some embodiments, the oral care composition comprises about 0.1 wt. % to about 1.0 wt. % of the sweetening agent.

In an embodiments, this disclosure provides a method for using the silk fibroin fragments based oral care products comprising the steps of: a) administering the oral care product to the oral cavity, b) contacting the oral care composition with the teeth and/or gums for a period of time, c) removing the remaining oral care composition from the mouth, and d) optionally rinsing the oral cavity with a liquid.

In some embodiments, the silk fibroin fragment based oral care product is in solid to flowable form, a tooth brush or the like may advantageously be used for contacting the oral care product with the teeth and/or gums. In some embodiments, the silk fibroin fragment based oral care product is a liquid product, the contacting with the teeth and/or gum may take place by rinsing the oral cavity.

In some embodiments, the time of contact in step b) is about 1 minute to 5 minutes. In some embodiments, the time of contact in step b) is about 1 minute. In some embodiments, the time of contact in step b) is about 2 minutes. In some embodiments, the time of contact in step b) is about 3 minutes.

After use, the silk fibroin protein fragment based oral care product may be removed from the mouth in any suitable way, e.g. by spitting it out. Optionally the mouth may be rinsed with a liquid, such as tap water.

### (1) Toothpaste

In some embodiments, this disclosure provides a silk toothpaste/gel composition comprising the silk fibroin protein fragments and an orally acceptable carrier as described herein. In some embodiments, the orally acceptable carrier is an aqueous liquid carrier comprising water. In some embodiments, the silk toothpaste/gel composition further comprises an ingredient selected from the group consisting of abrasive polishing materials, foaming agents, flavoring agents, humectants, binders, thickeners, sweetening agents, whitening/bleaching/stain removing agents, rhamnolipid, and combinations thereof.

In some embodiments, the silk toothpaste/gel comprises one or more of oral care active agents selected from the group consisting of silk solution or silk fibroin protein fragment composition as described above (from about 0.5 wt. % to 10.0 wt. %), a dental abrasive (from about 6.0 wt. % to about 50.0 wt. %), a surfactant (from about 0.5 wt. % to about 10.0 wt. %), a thickening agent (from about 0.1 wt. % to about 5 wt. %), a humectant (from about 10.0 wt. % to about 55.0 wt. %), a flavoring agent (from about 0.04 wt. % to about 2.0 wt. %), a sweetening agent (from about 0.1 wt. % to about 3.0 wt. %), a solid particle (from about 0.01 wt. % to about 5.0 wt. %), and water (from about 2.0 wt. % to about 45.0 wt. %), wherein the % is a weight percent of the ingredient by the total weight of the silk toothpaste/gel composition. In some embodiments, the silk toothpaste/gel further comprises one or more of an anticaries agent (from about 0.05% to about 0.3% as fluoride ion), and an anticalculus agent (from about 0.1% to about 13%), wherein the % is a weight percent of the ingredient by the total weight of the silk toothpaste/gel composition.

In some embodiments, the silk toothpaste/gel comprises one or more of the following ingredients: about 0.5 wt. % to about 10.0 wt. % of silk fibroin protein fragments; about 10.0 wt. % to 70.0 wt. % of abrasive material (e.g. silica, expanded perlite, bioglasses); 0 to about 80.0 wt. % of humectant (e.g., glycerol); about 0.1 wt. % to about 20.0 wt. % of thickener (e.g., carboxymethylcellulose); about 0.01 wt. % to 10.0 wt. % of binder; about 0.1 wt. % to about 5.0 wt. % of sweetener (sorbitol); 0 to about 15.0 wt. % of foaming agent (e.g., lauryl sulfate); 0 to about 5.0 wt. % of whitening agent (e.g. chlorine dioxide, bleach, peroxide); about 20.0 wt. % to about 30.0 wt. % of anticarie agent (e.g., hydrogen phosphate dihydrate); and about 20.0 wt. % to about 40.0 wt. % purified water, wherein the % is a weight percent of the ingredient by the total weight of the silk toothpaste/gel composition.

In some embodiments, the abrasive polishing material is selected from the group consisting of alpha alumina trihydrate, magnesium trisilicate, magnesium carbonate, kaolin, aluminosilicates, such as calcined aluminum silicate and aluminum silicate, calcium carbonate, zirconium silicate, powdered plastics, powdered polyvinyl chloride, powdered polyamides, powdered polymethyl methacrylate, powdered polystyrene, powdered phenol-formaldehyde resins, powdered melamine-formaldehyde resins, powdered urea-formaldehyde resins, powdered epoxy resins, powdered polyethylene, silica xerogels, silica hydrogels, silica aerogels, expanded perlite, non-expanded perlite, bioglasses, and combinations thereof. In some embodiments, additional abrasive agent is selected from the group consisting of calcium pyrophosphate, water-insoluble alkali metaphosphates, dicalcium phosphate and/or its dihydrate, dicalcium orthophosphate, tricalcium phosphate, particulate hydroxyapatite, and combinations thereof.

In some embodiments, the silk toothpaste/gel comprises about 10.0 wt. % to about 70.0 wt. % of the abrasive agent.

In some embodiments, the silk toothpaste/gel further comprises lyophilized silk fibroin protein fragments powder prepared by freeze drying the silk solution as described above. In some embodiments, the silk toothpaste/gel comprises about 0.01 wt. % to about 5.0 wt. % of the lyophilize silk fibroin protein fragment powders. In some embodiments, the lyophilize silk fibroin protein fragment powders has a median particle size ranging from about 0.5 μm to about 10 μm. In some embodiments, the lyophilize silk fibroin protein fragment powders has a median particle size selected from the group consisting of about 0.5 μm, about 1.0 μm, about 1.5 μm, about 2.0 μm, about 2.5 μm, about 3.0 μm, about 3.5 μm, about 4.0 μm, about 4.5 μm, about 5.0 μm, about 5.5 μm, about 6.0 μm, about 6.5 μm, about 7.0 μm, about 7.5 μm, about 8.0 μm, about 8.5 μm, about 9.0 μm, about 9.5 μm, and about 10.0 μm.

### (2) Mouth Wash and Mouth Rinse

In some embodiments, this disclosure provides a silk mouthwash and/or mouth rinse composition comprising the silk fibroin protein fragments and an orally acceptable carrier as described above. In some embodiments, the orally acceptable carrier comprise a water/alcohol solution as plaque removing liquids. In some embodiments, silk mouthwash and/or mouth rinse composition further comprises an additive selected from the group consisting of flavorant, humectant, sweetener, foaming surfactant, colorant, and combinations thereof.

In some embodiments, the silk mouthwash and/or mouth rinse composition is formulated as a product selected from the group consisting of mouth sprays, mouthwash, and mouth rinse. In some embodiments, the silk mouthwash and/or mouth rinse composition comprises one or more of the components selected from the group consisting of about 0.6 wt. % to about 6.0 wt. % of silk fibroin protein fragments as described above; about 45.0 wt. % to about 95.0 wt. % of water; 0% to about 25.0 wt. % of ethanol; 0% to about 50.0 wt. % of a humectant; about 0.01 wt. % to about 7.0 wt. % of a surfactant; about 0.04 wt. % to about 2.0 wt. % of a flavoring agent; about 0.3 wt. % to about 3.0 wt. % of a sweetening agent; about 0.001 wt. % to about 0.5 wt. % of a coloring agent, wherein the % is a weight percent of the ingredient by the total weight of the silk toothpaste/gel composition. In some embodiments, the silk mouthwash and/or mouth rinse composition optionally comprises about 0.05 wt. % to about 0.3 wt. % of fluoride ion as an anticaries agent and/or about 0.1 wt. % to about 3.0 wt. % of an anticalculus agent, wherein the % is a weight percent of the ingredient by the total weight of the silk toothpaste/gel composition.

### (3) Remineralizing Toothpaste

Dental caries, also known as tooth decay or cavities, is a breakdown of teeth due to acids made by bacteria. Remineralization is encouraged to prevent and treat dental caries. Remineralization occurs when a mineral is added to the teeth to replace mineral components that have been depleted from the teeth. Fluoride has been the cornerstone for caries prevention. Fluoride toothpaste is the most widely used fluoride modality. However, the conventional fluoride toothpaste has a significant caries-preventive effect only at concentrations of 1,000 parts per million (ppm) or higher. However, this high concentration of fluoride ion in the toothpaste is associated with an increased risk of fluorosis when used by young children under the age of 6, particularly before two years old.

Non-fluoride topical remineralizing agents containing calcium and/or phosphate has been investigated and showed the potential as an alternative to fluoride or as an adjunct to fluoride to enhance its effectiveness at lower fluoride concentration. Casein phosphoprotein-amorphous calcium phosphate (CPP-ACP) is currently most commonly used in clinic. Calcium phosphate (Ca—P) compounds have been added to a variety of topical delivery vehicles and are commercially available in toothpaste, chewing gum, varnish, and mouth rinse. (Zero, D.T. Dentifrices, mouthwashes, and remineralization/caries arrestment strategies. BMC Oral Health 6 Suppl. 1, S9 (2006)). The significant problem with CPP-ACP is its low solubility in acidic microenvironment where tooth demineralization occurs. The problem of stabilizing calcium and phosphate ions so that bioavailable Ca—P can be delivered when needed is a major challenge that impedes a large scale, population-based utilization of Ca—P-based products for caries prevention and control. There is a continued need to for a better delivery system of soluble calcium and phosphate to the teeth.

In some embodiments, this disclosure provides a silk tooth remineralization composition comprising a therapeutically effective amount of a remineralizing agent, the silk fibroin protein fragments and an orally acceptable carrier as described herein.

In some embodiments, the remineralizing agent is selected from the group consisting of fluoride, calcium and/or phosphate, amorphous calcium phosphate (ACP), tricalcium phosphate, casein phosphoprotein-ACP, bioactive glass, calcium sodium phosphosilicate, arginine bicarbonate-calcium carbonate complex, calcium acetate, CaCl₂), calcium pantothenate, calcium ascorbate, calcium gluconate, calcium lactate, calcium acetylacetonate, calcium lactobionate, calcium citrate, calcium α-D-heptagluconate, calcium benzoate, saccharin calcium and/or ascorbic acid calcium, and combinations thereof. In some embodiments, the remineralizing agent is amorphous hydroxyapatite. In some embodiments, the remineralizing agent is amorphous calcium phosphate.

In some embodiments, the remineralizing agent comprises calcium and/or phosphate having a median particle size from 1 μM to about 250 μM. In some embodiments, the remineralizing agent comprises calcium and/or phosphate having a median particle size from 1 μM to about 100 μM. In some embodiments, the remineralizing agent is a mixture of a calcium ion source compound and a phosphate ion source compound having a molar ratio of between about 1:1 to about 1:2 of calcium to phosphate.

In some embodiments, the silk tooth remineralization composition comprises about 1.0 wt. % to about 10.0 wt. % of calcium phosphate. In some embodiments, the silk tooth remineralization composition comprises calcium phosphate at an amount selected from the group consisting of about 1.0 wt. %, 2.0 wt. %, 3.0 wt. %, 4.0 wt. %, 5.0 wt. %, 6.0 wt. %, 7.0 wt. %, 8.0 wt. %, 9.0 wt. %, and 10.0 wt. % by the total weight of the silk tooth remineralization composition.

In some embodiments, the silk tooth remineralization composition further comprises fluoride ion at a concentration of about 500 ppm to about 10,000 ppm. In some embodiments, the silk tooth remineralization composition further comprises fluoride ion at a concentration of about 1000 ppm to about 7,500 ppm. In some embodiments, the silk tooth remineralization composition further comprises fluoride ion at a concentration of about 1,000 ppm to about 5,000 ppm. In some embodiments, the silk tooth remineralization composition further comprises fluoride ion at a concentration of about 50 ppm to about 1,000 ppm. In some embodiments, the silk tooth remineralization composition further comprises fluoride ion at a concentration of about 100 pm to about 500 ppm. In some embodiments, the silk tooth remineralization composition further comprises fluoride ion at a concentration selected from the group consisting of about 50 ppm, about 60 ppm, about 70 ppm, about 80 ppm, about 90 ppm, about 100 ppm, about 110 ppm, about 120 ppm, about 130 ppm, about 140 ppm, about 150 ppm, about 160 ppm, about 170 ppm, about 180 ppm, about 190 ppm, about 200 pm, about 210 ppm, about 220 ppm, about 230 ppm, about 240 ppm, about 250 ppm, about 260 ppm, about 270 ppm, about 280 ppm, about 290 ppm, about 300 pm, about 310 ppm, about 320 ppm, about 330 ppm, about 340 ppm, about 350 ppm, about 360 ppm, about 370 ppm, about 380 ppm, about 390 ppm, about 400 pm, about 410 ppm, about 420 ppm, about 430 ppm, about 440 ppm, about 450 ppm, about 460 ppm, about 470 ppm, about 480 ppm, about 490 ppm, about 500 pm, about 510 ppm, about 520 ppm, about 530 ppm, about 540 ppm, about 550 ppm, about 560 ppm, about 570 ppm, about 580 ppm, about 590 ppm, about 600 pm, about 610 ppm, about 620 ppm, about 630 ppm, about 640 ppm, about 650 ppm, about 660 ppm, about 670 ppm, about 680 ppm, about 690 ppm, about 700 pm, about 710 ppm, about 720 ppm, about 730 ppm, about 740 ppm, about 750 ppm, about 760 ppm, about 770 ppm, about 780 ppm, about 790 ppm, about 800 pm, about 810 ppm, about 820 ppm, about 830 ppm, about 840 ppm, about 850 ppm, about 860 ppm, about 870 ppm, about 880 ppm, about 890 ppm, about 900 pm, about 910 ppm, about 920 ppm, about 930 ppm, about 940 ppm, about 950 ppm, about 960 ppm, about 970 ppm, about 980 ppm, about 990 ppm, and about 1000 pm.

In some embodiments, the fluoride ion forms complex with the silk fibroin protein fragments obtained by mixing the fluoride source compound in silk solution as described above at pH of 4.5 to 5.5 prior to the addition to the other components of the silk tooth remineralization composition.

In some embodiments, the remineralizing agent is encapsulated by silk fibroin protein hydrogel microparticles formed from the silk solution as described above. The silk fibroin protein hydrogel microparticle encapsulated remineralizing agent is prepared by the following steps: (1) providing the silk solution as described above, (2) sonicating the silk solution, optionally with vitamin C, or glycerin is added to the silk solution; (3) adding the remineralizing agent to the silk solution of step 2 just after sonication and the solution is mixed by inversion, in which the sol-gel transition is initiated, but the silk fibroin protein fragments are still in the solution state; (4) immediately after mixing, the remineralizing agent-silk solution mixture of step (3) is added dropwise to a sunflower oil bath in a petri dish and incubating at ambient condition overnight to allow complete sol-gel transition; (5) removing silk hydrogel microparticle encapsulated remineralizing agent from the oil. The emulsion of sunflower oil in silk solution is prepared with silk fibroin protein fragments having a concentration at about 0.6% (w/v), 1.2% (w/v), 2.4% (w/v), 4.0% (w/v), and 6.0% (w/v), and volume ratio of oil to the silk solution is at 4:1, 2:1, 3:2, 1:1, 2:3, 1:2, and 1:4. Additional oils suitable for making the silk hydrogel microparticle may include squalane, jojoba oil, and combinations thereof.

In some embodiments, the silk fibroin solution used to encapsulate the remineralizing agent comprises silk fibroin protein fragments having an average weight average molecular weight selected from between about 40 kDa to about 45 kDa, about 45 kDa to about 50 kDa, about 60 kDa to about 100 kDa, about 80 kDa to about 144 kDa, about 135 kDa to about 140 kDa, about 145 Da to about 150 Da, about 150 kDa to about 155 kDa, about 155 kDa to about 160 kDa, about 160 kDa to about 165 kDa, about 165 kDa to about 170 kDa, about 170 kDa to about 175 kDa, about 175 kDa to about 180 kDa, about 180 kDa to about 185 kDa, about 185 kDa to about 190 kDa, about 190 kDa to about 195 kDa, about 195 kDa to about 200 kDa, about 200 kDa to about 205 kDa, about 205 kDa to about 210 kDa, about 210 kDa to about 215 kDa, about 215 kDa to about 220 kDa, about 220 kDa to about 225 kDa, about 225 kDa to about 230 kDa, about 230 kDa to about 235 kDa, about 235 kDa to about 240 kDa, about 245 kDa to about 250 kDa, about 250 kDa to about 255 kDa, about 255 kDa to about 260 kDa, about 260 kDa to about 265 kDa, about 265 kDa to about 270 kDa, about 270 kDa to about 275 kDa, about 275 kDa to about 280 kDa, about 285 kDa to about 290 kDa, about 290 kDa to about 295 kDa, about 295 kDa to about 300 kDa, about 300 kDa to about 305 kDa, about 305 kDa to about 310 kDa, about 310 kDa to about 315 kDa, about 315 kDa to about 320 kDa, about 320 kDa to about 325 kDa, about 325 kDa to about 330 kDa, about 330 kDa to about 335 kDa, about 335 kDa to about 340 kDa, about 340 kDa to about 345 kDa, about 345 kDa to about 350 kDa, and a polydispersity of 1 to about 5.0, or about 1.5 to about 3.0.

In some embodiments, the silk hydrogel microparticle encapsulated remineralizing agent having a median particle size of about 1 μm to about 75 μm. In some embodiments, the silk hydrogel microparticle encapsulated remineralizing agent having a median particle size of 1 μm to about 10 μm. In some embodiments, the silk hydrogel microparticle encapsulated remineralizing agent having a median particle size selected from the group consisting of about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, and about 75 μm.

In some embodiments, the remineralizing agent protected by the silk hydrogel microparticle comprises about 1.0 wt. to about 50.0 wt. % encapsulated remineralizing agent, and about 10.0 wt. % to about 70.0 wt. % silk fibroin protein fragments by the total weight of the silk hydrogel microparticle.

In some embodiments, the silk tooth remineralization composition contains a therapeutically effective amount of the silk hydrogel microparticle encapsulated remineralizing agent ranging from about 0.01 wt. % to about 40.0 wt. % by the total weight of the tooth remineralization composition. In some embodiments, the silk tooth remineralization composition contains a therapeutically effective amount of the silk hydrogel microparticle encapsulated remineralizing agent ranging from about 1.0 wt. % to about 30.0 wt. % by the total weight of the tooth remineralization composition. In some embodiments, the silk tooth remineralization composition contains a therapeutically effective amount of the silk hydrogel microparticle encapsulated remineralizing agent ranging from about 5.0 wt. % to about 20.0 wt. % by the total weight of the tooth remineralization composition.

In some embodiments, the silk tooth remineralization composition is formulated as a remineralizing gel, a remineralizing mouthwash, a remineralizing tooth powder, a remineralizing chewing gums and/or whitening strip, a remineralizing varnish, a remineralizing cream, a remineralizing lozenge, or a remineralizing toothpaste.

### (4) Oral Hygiene Powder, Pellet, Tablet or Capsule

Travel is becoming increasingly difficult with restrictions being placed on luggage weight, checked luggage numbers, and liquids located in carry-ons. When traveling, large tubes and bottles can be quite cumbersome, taking up far too much space in one's luggage or carryon. There remains a need for an oral hygiene powder, capsule/tablet arranged to contain a dosage of mouthwash or toothpaste and/or active ingredients so that a user can compactly carry and store mouthwash, toothpaste and/or active ingredients.

In one embodiment, the disclosure provides a silk oral care composition comprising SPF as defined herein, including, without limitation, silk fibroin protein and silk fibroin fragments, having a polydispersity ranging from 1 to about 5; from 0 to 500 ppm lithium bromide; from 0 to 500 ppm sodium carbonate; a dental care active agent; and one or more dentally acceptable excipients. In some embodiments, the silk fibroin fragments have an average weight average molecular weight selected from between about 1 kDa to about 5 kDa, from between about 5 kDa to about 10 kDa, from between about 6 kDa to about 17 kDa, from between about 10 kDa to about 15 kDa, from between about 15 kDa to about 20 kDa, from between about 17 kDa to about 39 kDa, from between about 20 kDa to about 25 kDa, from between about 25 kDa to about 30 kDa, from between about 30 kDa to about 35 kDa, from between about 35 kDa to about 40 kDa, from between about 39 kDa to about 80 kDa, from between about 40 kDa to about 45 kDa, from between about 45 kDa to about 50 kDa, from between about 60 kDa to about 100 kDa, and from between about 80 kDa to about 144 kDa.

In some embodiments, this disclosure provides silk oral care articles comprising the silk powders as described above and one or more dentally acceptable excipients. The silk powder may function as whitening agent to modify the color of the teeth. The silk powder may function as binder to form flowable powder in the finished product. In some embodiments the silk oral care article is an oral care product selected from the group consisting of tooth powder, toothpaste tablet, toothpaste pellet, toothpaste pod, mouthwash tablet, mouthwash pellet, and mouthwash pod.

In some embodiments, this disclosure provides tooth powders comprising the silk powder as described above (See Example 8 below for various methods of preparation) and one or more dentally acceptable excipients selected from the group consisting of baking soda, calcium carbonate, calcium powder, sodium bicarbonate, activated charcoal, diatomaceous earth, magnesium carbonate, dicalcium phosphate, tartaric acid, antioxidant, fluoride (e.g., sodium monoflurophosphate), sweetener (e.g., sodium saccharin, xylitol), clay (e.g., bentonite, kaolin, montmorillonite), sea salt, essential oil (peppermint, eucalyptus, clove, wintergreen, spearmint, and oregano), surfactant (e.g., sodium lauryl sulfate, sodium cocoyl isethionate), whitening agent (e.g., activated charcoal, silk powder), and herbs (e.g., cloves, mint, sage, cinnamon).

In some embodiments, the amount of the silk powder in the tooth powder ranges from about 1.0 wt. % to about 20.0 wt. % by the total weight of the tooth powder. In some embodiments, the amount of the silk powder in the tooth powder ranges from about 1.0 wt. % to about 15.0 wt. % by the total weight of the tooth powder. In some embodiments, the amount of the silk powder in the tooth powder ranges from about 1.0 wt. % to about 10.0 wt. % by the total weight of the tooth powder. In some embodiments, the amount of the silk powder in the tooth powder ranges from about 1.0 wt. % to about 6.0 wt. % by the total weight of the tooth powder.

In some embodiments, this disclosure provides a method of making tooth powder comprising the step of blending the silk powder with the one or more dentally acceptable excipients in a mixing device. In some embodiments, the tooth powder may be in the form of flowable granules formed by wet granulation using the silk solution described above as binder solution. In some embodiments, the tooth powder may be in the form of flowable granules formed by melt granulation. In some embodiments, the tooth powder may be in the form of flowable granules formed by dry granulation.

The use of tooth powder is similar to brushing teeth with toothpaste, for example, first wetting the toothbrush and dipping it into the powder, or use a small squirt bottle to squirt the powder onto a wet toothbrush.

In an embodiment, this disclosure provides toothpaste tablets/pellets with mouthwash and/or toothpaste, oral/dental care active agent(s) for direct delivery and dissolution into the mouth. In an embodiment, this disclosure provides a method of cleaning teeth by placing the toothpaste tablets/pellets into the mouth, wherein the toothpaste tablet/pellet is substantially dissolved when contacted with saliva, water, or both, thereby changing the form into liquid.

In some embodiments, this disclosure provides a compressed toothpaste tablet or toothpaste pellet comprising the silk powder described above, plurality of abrasive particles, and one or more dentally acceptable excipients selected from the group consisting of disintegrant (e.g., microcrystalline cellulose), filler, glidant (e.g., silicon dioxide), lubricant (e.g. magnesium stearate), cleansing surfactant (sodium lauryl glutamate), sweetener (sodium saccharin, xylitol, stevioside), flavoring agent (e.g., mint), essential oil (peppermint, eucalyptus, clove, wintergreen, spearmint, and oregano), breath freshening agent (e.g., menthol), binder, pH adjusting agent (e.g., sodium bicarbonate), effervescent component (e.g., sodium bicarbonate and citric acid), fluoride (e.g. sodium fluoride, sodium monoflurophosphate), whitening agent, therapeutic agent, vitamin, cooling agent, mineral, antimicrobial agent, and combinations thereof.

In some embodiments, the filler is selected from the group consisting of lactose, glucose, maltodextrins, sucrose, sorbitol, xylitol, mannitol, and maltitol, dicalcium phosphate, and combinations thereof. In some embodiments, the filler is dicalcium phosphate. In some embodiments, the filler is lactose. In some embodiments, the filler is xylitol.

In some embodiments, the binder is selected from the group consisting of xanthan gum, carrageenan, pregelatinized starch, agars, locust bean gums, guar gums, tara gums, carrageenan, alginate, xanthan, dextran, sodium carboxymethyl cellulose, sodium carboxymethyl hydroxyethyl cellulose, gum karaya, gum arabic, gum tragacanth, and combinations thereof.

In some embodiments, the whitening agent is selected from the group consisting of activated charcoal, silk powder, peroxide, hydrogen peroxide, talc, mica, magnesium carbonate, calcium carbonate, calcium pyrophosphate, baking soda, Icelandic moss, bamboo, sodium hexametaphosphate, magnesium silicate, aluminum magnesium carbonate, silica, titanium dioxide, zinc oxide, red iron oxide, brown iron oxide, yellow iron oxide, black iron oxide, ferric ammonium ferrocyanide, manganese violet, ultramarine, nylon powder, polyethylene powder, methacrylate powder, polystyrene powder, crystalline cellulose, starch, titanated mica, iron oxide titanated mica, bismuth oxychloride, and combinations thereof.

In some embodiments, the therapeutic agent is selected from the group consisting of eugenol, anticaries agent, anticalculus agent, antimicrobial agent, anti-inflammatory agent, and combinations thereof.

In some embodiments, the vitamin is selected from the group consisting of vitamins B1, vitamin B2, vitamin B6, vitamin B12, vitamin C, and vitamin E, coenzyme (CQ10)), and combination thereof.

In some embodiments, the cooling agent is selected from the group consisting of N-ethyl-p-menthane-3-carboxamide, N,2,3-trimethyl-2-isopropylbutanamide, and combinations thereof.

In some embodiments, the mineral is selected from the group consisting of calcium, magnesium, chromium, zinc, selenium, iron, and combinations thereof.

In some embodiments, the antimicrobial agent suitable for treating gum diseases is selected from the group consisting of chlorhexadine, tetracycline, cetyl pyridinium chloride, benzalkonium chloride, cetyl pyridinium bromide, methylbenzoate, propylbenzoate, and combinations thereof.

In some embodiments, the anticalculus agents is selected from the group consisting of diphosphonate (e.g., 1-azocycloheptane-2,2-diphosphonate (AHP) and ethane-1-hydroxy-1,1-diphosphonate (EHDP)), sodium zinc citrate, phosphocitrate, tripolyphosphate, linear polycarboxylate (LPC), pyrophosphate, olyphosphate, disodium dihydrogen pyrophosphate (Na₂H₂P₂O₇), tetrasodium pyrophosphate (Na₄P₂O₇), anhydrous tetrapotassium pyrophosphate (K₄P₂O₇), hydrated K₄P₂O₇, and combinations thereof.

In some embodiments, the abrasive is selected from the group consisting of silica, calcium carbonate, dicalcium orthophosphate dihydrate, calcium pyrophosphate, tricalcium phosphate, calcium polymetaphosphate, insoluble sodium polymetaphosphate, hydrated alumina, particulate condensation products of urea and formaldehyde abrasive, natural glass, perlite, bioglass, and combinations thereof. The plurality of the abrasive particles clean the teeth, remove debris, and/or remove the adhering layers of bacterial film without excessively abrading dentine from the teeth. In some embodiments, the plurality of the abrasive particles comprise silica and silk fibroin fragment powder. In some embodiments, the plurality of abrasive particles have mean particle size ranging from about 0.1 μm to about 30 m μm. In some embodiments, the plurality of abrasive particles have mean particle size ranging from about 5.0 μm and about 15.0 μm.

In some embodiments, the amount of the plurality of abrasive particles in the compressed toothpaste tablet or toothpaste pellet ranges from about 20.0 wt. % to about 80.0 wt. by the total weight of the compressed toothpaste tablet or toothpaste pellet. In some embodiments, the amount of the silk powder in the compressed toothpaste tablet or toothpaste pellet ranges from about 35.0 wt. % to about 70.0 wt. % by the total weight of the compressed toothpaste tablet or toothpaste pellet.

In some embodiments, the amount of the disintegrant in the compressed toothpaste tablet ranges from about 20.0 wt. % to about 80.0 wt. % by the total weight of the compressed toothpaste tablet. In some embodiments, the amount of the disintegrant in the compressed toothpaste tablet ranges from about 35.0 wt. % to about 65.0 wt. % by the total weight of the compressed toothpaste tablet.

In some embodiments, the amount of the silk powder in the compressed toothpaste tablet or toothpaste pellet ranges from about 0.5 wt. % to about 20.0 wt. % by the total weight of the compressed toothpaste tablet or toothpaste pellet. In some embodiments, the amount of the silk powder in the compressed toothpaste tablet or toothpaste pellet ranges from about 1.0 wt. % to about 15.0 wt. % by the total weight of the compressed toothpaste tablet or toothpaste pellet. In some embodiments, the amount of the silk powder in the compressed toothpaste tablet or toothpaste pellet ranges from about 1.0 wt. % to about 10.0 wt. % by the total weight of the compressed toothpaste tablet or toothpaste pellet. In some embodiments, the amount of the silk powder in the compressed toothpaste tablet or toothpaste pellet ranges from about 1.0 wt. % to about 6.0 wt. % by the total weight of the compressed toothpaste tablet.

In some embodiments, the amount of the binder in the compressed toothpaste tablet or toothpaste pellet ranges from about 0.2 wt. % to about 6.0 wt. % by the total weight of the compressed toothpaste tablet or toothpaste pellet. In some embodiments, the amount of the binder in the compressed toothpaste tablet or toothpaste pellet ranges from about 1.0 wt. % to about 5.0 wt. % by the total weight of the compressed toothpaste tablet or toothpaste pellet.

The unit dose of the compressed toothpaste tablet or toothpaste pellet is for a single use at an amount recommended by the dentist. In contrast to the conventional toothpaste tube, the compressed toothpaste tablet or toothpaste pellet could be packaged in a peel away packet having discrete compartment, or stored in a sachet, a bottle, or any suitable container.

The compressed toothpaste tablet or toothpaste pellet described herein may be prepared by granulation followed by compression on any pharmaceutical tablet press machines.

The toothpaste pellets described herein may be prepared by extrusion and spheronization, powder layering, liquid layering, and pelletization by melt and wet granulation.

In some embodiments, this disclosure provides mouthwash/toothpaste pods comprising a silk gel as outer shell and an inner cavity with oral/dental care active agents housed therein for direct delivery into a user's mouth.

The outer shell is configured to dissolve or break down in saliva in a mouth of a user, and may be hastened by drinking water at the time of insertion, or by biting and chewing on the outer shell to cause penetration and breakdown of the outer shell into small particles. The oral/dental care active agents encapsulated within the inner cavity of the pods are released as the outer shell dissolves or is penetrated, and directly infuses the mouth and oral tissue to deliver the active ingredients within the oral cavity. In some embodiments, the oral/dental care active agents are selected from the group consisting of toothpaste, hydrogen peroxide, mouthwash, herbal ingredients for providing a soothing effect to the mouth, numbing agent, vitamin, mineral, therapeutic agent, nutrient supplement, and/or fluoride supplement. The outer shell of the pod may be composed of effervescent ingredients comprising a mixture of silk gel, citric acid and sodium bicarbonate, a silk-gelatin gel composition to microencapsulate the oral care active agents, a combination of gelatin and silk gel, a combination of silk gel and carrageenan, a combination of silk gel and modified forms of starch and cellulose, or a combination of silk gel and a gum compound.

The dissolution process of the pod can be accelerated by biting and breaking it into smaller particles and/or by drinking water when the pod is inserted into the mouth.

The discretely packed oral hygiene products described above are more hygienic than the conventional toothpaste tubes by eliminating brush-to-nozzle contact, and less mess is produced from toothpaste residue that builds up on nozzles and elsewhere.

In some embodiments, the compressed tooth tablet/pellet above is a mucoadhesive tablet/pellet for localized controlled delivery of dental care and/or oral care active agent. In some embodiments, the mucoadhesive tablets/pellets have flat or oval shape with a diameter of approximately 5-8 mm. Unlike the conventional tablets/pellets, mucoadhesive tablets/pellets allow for drinking and speaking without major discomfort. They soften, adhere to the mucosa, and are retained in position until dissolution and/or release is complete. Mucoadhesive tablets/pellets also offer efficient absorption and enhanced bioavailability of the oral/dental care active agent due to a high surface to volume ratio and facilitates a much more intimate contact with the mucus layer.

### (5) Oral Care Patches

In some embodiments, this disclosure provides silk oral care patches comprising a nonwoven sheet impregnated or coated with the oral care composition as described above, and a backing layer. In some embodiments, the silk oral care composition is a solution, a gel, a paste, a powder, an emulsion, or a suspension. In some embodiments, the silk oral care composition is a solution or a gel as described in Example 1 and Example 9 below.

In some embodiments, the nonwoven sheet comprises meltblown, spunbond, bonded carded, bicomponent, or crimped fibers. In some embodiments, the nonwoven sheet comprise meltblown microfibers have smaller than 10 microns in mean diameter, In some embodiments, the nonwoven sheet comprises spunbond fibers having mean diameter of about 7 μm to about 40 μm.

In some embodiments, the nonwoven sheet is made of a polymer selected from the group consisting of polypropylene (PP), thermoplastic polyurethane (TPU), polypropylene (PP), Nylone and combinations thereof. In some embodiments, the silk oral care patches are configured to fit the size and contour of the patient's teeth. In some embodiments, the silk oral care patches further comprise a mucoadhesive layer attached to one side of the nonwoven sheet, wherein the mucoadhesive layer comprises a mucoadhesive polymer selected from the group consisting of chitosan, hyaluronic acid, xanthan gum, and combinations thereof.

In some embodiments, the silk oral care composition comprises an oral care active agent selected from the group consisting of whitening agent, remineralization agent, antibiotic, antifungal agent, anesthetic agent, antiviral agent, anti-ulcerative agent, anti-analgesic agent, anti-inflammatory agent, anti-allergic agent, antimicrobial agent, silk fibroin derived amino acid, and combinations thereof.

In some embodiments, the backing layer comprises a polymer selected from the group consisting of polyurethane (PU), polyethylene (PE), polyesters, nylon, polyvinyl alcohol (PVA), polylactic acid (PLA), chitosan, polyvinyl methyl ether/maleic acid copolymer (PVM/MA copolymer), polyethylene oxide/polypropylene oxide copolymer (PEO/PPO copolymer), polyvinylpyrrolidone-vinyl acetate copolymer (PVP/VA copolymer), polyethylene oxide (Polyox), polyvinylpyrrolidone (PVP), γ-polyglycolic acid (γ-PGA), polyquaternium, carboxypolymethylene, carboxymethyl cellulose (CMC), hydroxypropyl methylcellulose, hydroxyethylcellulose, hydroxypropylcellulose, gelatin, alginate, regenerated silk fiber, or a mixture thereof.

The silk oral care patch described herein can be packed in series as a tape bundle or discrete packet. The silk oral care patch described herein can be used for treating disease by attaching the patch onto a target tissue suffering from oral disease. The backing layer is removed after the silk oral patch is affixed onto the target tissue. The target tissue can be one single tooth, a plurality of teeth, a mucosal tissue, and their combinations. The oral disease may be aphthous ulcers, or periodontitis.

The conventional oral patches have poor adhesion to tooth tissue, whereas the silk oral care patch described herein has high affinity to the target tissue due to the fact that the structure and content of amino acids in silk fibroin protein are very similar to the tissue of the human body. The enhanced adhesion of the silk oral patch to the dental tissue is resulted from the electrostatic interactions between the silk fibroin peptide chains and the tissue surface, for example, hydrogen bond via hydrophilic functional groups —NH₂ (arginine), —OH (serine), and —COOH (glutamic acid) of silk fibroin fragments, ionic interaction, hydrophobic van de Waals interactions.

### (6) Mucoadhesive Gel and Ointment

In some embodiments, this disclosure provides a silk oral care product comprising mucoadhesive gel or ointment formed from the oral care compositions described above and an oral/dental care active agent as described above, wherein the silk fibroin fragments exhibit mucoadhesive property. Semisolid dosage forms, such as gels and ointments, have the advantage of easy dispersion throughout the oral mucosa. However, the dosing for the oral/dental care active agent from semisolid dosage forms may not be as accurate as from tablets, patches, or films. Poor retention of the gel or ointment at the site of application has been overcome by using mucoadhesive formulations as described herein. In some embodiments, the mucoadhesive gel or ointment further comprises one or more mucoadhesive polymers selected from the group consisting of sodium carboxymethylcellulose, carbopol, hyaluronic acid, xanthan gum, and combinations thereof.

In some embodiments, the mucoadhesive gel or ointment comprises the silk hydrogels as described in the Example 9 below for buccal delivery of the oral/dental care active agent. The silk hydrogel physically entraps the therapeutic agent for subsequent slow release by diffusion. The application of mucoadhesive gel or ointment provides an extended retention time in the oral cavity, adequate oral/dental care active agent penetration, and high efficacy.

In some embodiments, the oral/dental care active agent is selected from the group consisting of fluoride salt (sodium fluoride, stannous fluoride, sodium monofluorophosphate, ammonium fluoride), strontium salt, potassium salt, stannous fluoride, phosphate fluoride, hydrogen peroxide, potassium chlorate, potassium permanganates, clove oil, wintergreen, pontacaine, hemostatic agent, zinc salt, antioxidant, antibiotic, antimicrobials, antiseptic agent, antifungal agent, anesthetic agent, antiviral agent, anti-ulcer active agent, anti-allergic agent, anti-analgesic agent, analgesic, hemostatic agent, anti-inflammatory agent (e.g., flubiprofen, naproxen, ketoprofen, aspirin), growth factor, anti-tumor agent, desensitizing agent, hormones, Vitamin, amino acid, vaccine, caffeine, monoclonal antibody, enzyme, and combinations thereof.

In some embodiments, the zinc salt is selected from the group consisting of the zinc salt is selected from the group consisting of zinc chloride, zinc acetate, zinc phenol, sulfonate, zinc borate, zinc bromide, zinc nitrate, zinc glycerophosphate, zinc benzoate, zinc carbonate, zinc citrate, zinc hexafluorosilicate, zinc diacetate trihydrate, zinc oxide, zinc peroxide, zinc salicylate, zinc silicate, zinc stannate, zinc tannate, zinc titanate, zinc tetrafluoroborate, zinc gluconate, zinc glycinate, and combinations thereof.

In some embodiments, the desensitizing agent is one or more strontium salts selected from the group consisting of strontium chloride, strontium bromide, strontium iodide, strontium acetate, strontium edetate, strontium nitrate, strontium salicylate, strontium lactate, potassium nitrate (KNO₃), citric acid, citrate salt, and combinations thereof. Since desensitizing agent must penetrate into the pores of a person's teeth in order to reach the nerves within the dental pulp, the mucoadhesive gel or ointment described herein provides prolonged contact between the desensitizing agent and the person's teeth.

In some embodiments, the antioxidant is selected from the group consisting of vitamin A, vitamin E, pyruvate B-carotene, selenium, N-acetylcysteine, vitamin C, superoxide dismutase (SOD), catalase, glutathione peroxidase, glutathione reductase, and combinations thereof.

In some embodiments, the oral/dental care agent is encapsulated within nanoparticles, microparticles, or microcapsules. In some embodiments, the oral/dental care agents described above are encapsulated within a silk fibroin protein fragment particle, wherein the silk fibroin protein fragment forms the matrix or forms a particle shell, and the oral/dental care agents may be embedded with the particle matrix or enclosed inside particle shell as an oil phase or an aqueous phase (microcapsules).

In some embodiments, various emulsion based particle preparation methods reported in the art such as emulsion/evaporation method may be used to prepare silk microparticles. In some embodiments, the silk solution, or the various silk fibroin protein fragments compositions as described above can be used to prepare the silk microparticle encapsulated oral/dental care agents.

In some embodiments, this disclosure provides a method of treating periodontitis in a subject suffering from the disorder comprising administering to the subject the mucoadhesive gel or ointment described above for the local delivery of oral/dental care agents to diseased tissue, wherein the periodontitis is an inflammatory and infectious disease that causes formation of pockets between the gum and the tooth, and can eventually cause loss of teeth. In some embodiments, the silk fibroin containing mucoadhesive gel or ointment is a highly viscous gel containing the silk fibroin fragments described above, carbopal and hydroxypropylcellulose for ointment dosage forms.

### (7) Dental Floss and Toothpick

In some embodiments, this disclosure provides silk oral care article comprising a uncoated dental floss impregnated or coated with the silk oral care compositions as described above. By coating the floss with the silk fibroin fragments described above, the lubricity of the floss is enhanced. The silk coated dental floss may optionally have flavoring agent to improve the organoleptic property of the floss.

In some embodiments, this disclosure provides a therapeutic floss or toothpick impregnated or coated with the therapeutic agent and the silk fibroin fragments compositions described above.

In some embodiments, this disclosure provides a method of treating oral and systemic diseases comprising providing the therapeutic dental floss or toothpick described above and rubbing the dental floss or toothpick against mouth tissue to release the therapeutic agent onto the tissue for penetration through tissue for systemic absorption. In some embodiments, the therapeutic dental floss or toothpick are applied for controlled or sustained therapeutic agent release.

In some embodiments, this disclosure provides a method of making therapeutic dental floss or toothpick comprising the steps of: (1) providing uncoated dental floss or toothpick; (2) preparing a coating preparation by dissolving or dispersing the therapeutic agent into the silk solution described above; (3) dipping or immersing the uncoated dental floss or toothpick into the coating preparation for a sufficient period of time; and (4) optionally drying the dental floss or toothpick.

In some embodiments, the toothpicks are usually tapered to a point at one or both ends and are made of wood, plastic, stiff paper, metal, ivory or other materials that provide sufficient rigidity to expel particles between the teeth, yet narrow enough to fit into the interdental spaces. Toothpicks may have various shapes: straight, bent, round, flat, curved, and various combinations. Toothpicks are dispensed singularly, individually wrapped in plastic, in matchbook dispensers, in rolls to be broken off and used, and some are stored in containers.

In some embodiments, the dental floss comprises plurality of threads selected from the group consisting of multiple braided threads of nylon, polyester, polyethylene, perfluoroalkoxy alkane (PFA), polytetrafluoroethylene (PTFE), polyethylene terephthalate (Dacron®), polylactic acid (PLA), polypropylene, and combinations thereof.

In some embodiments, the dental floss comprises plurality of silk doped polymer threads formed by hot melt extrusion from a base polymer and the silk powders as described above containing silk fibroin fragments having: (i) an average weight average molecular weight selected from between about 1 kDa and about 5 kDa, between about 5 kDa and about 10 kDa, between about 6 kDa and about 17 kDa, between about 10 kDa and about 15 kDa, between about 15 kDa and about 20 kDa, between about 17 kDa and about 39 kDa, between about 20 kDa and about 25 kDa, between about 25 kDa and about 30 kDa, between about 30 kDa and about 35 kDa, between about 35 kDa and about 40 kDa, between about 39 kDa and about 80 kDa, between about 40 kDa and about 45 kDa, between about 45 kDa and about 50 kDa, between about 60 kDa and about 100 kDa, and between about 80 kDa and about 144 kDa; and (ii) a polydispersity between 1.0 and about 5.0.

By doping the polymer fibers with the silk fibroin fragments described above, the strength and lubricity of the floss formed thereof may be enhanced.

In some embodiments, the polydispersity of the silk fibroin fragments is between 1 and about 1.5. In some embodiments, the polydispersity is between about 1.5 and about 3.0. In some embodiments, the polydispersity is between is between about 1.5 and about 2.0. In some embodiments, the polydispersity is between is between about 2.0 and about 2.5. In some embodiments, the polydispersity is between is between about 2.5 and about 3.0.

In some embodiments, the silk powder described herein has a glass transition temperature (T_{g}) of 217° C. (the glass transition takes place over a temperature range from 120° C. to 230° C.).

In some embodiments, the base polymer is selected from the group consisting of nylon, polyester, polyethylene, perfluoroalkoxy alkane (PFA), polytetrafluoroethylene (PTFE), polyethylene terephthalate (Dacron®), polypropylene, and combinations thereof.

In some embodiments, the silk doped polymer thread comprise monofilament. In some embodiments, the silk doped polymer thread comprise multifilament.

As sued herein, the term “silk doped polymer thread” refers to an article made of one or more continuous strands called filaments with each component filament running the whole length of the threads. The silk doped polymer threads may contain as few as 2 or 3 filaments and as many as 50.

In some embodiments, the base polymer content in the silk doped fiber is of about 10.0 wt. % by the total weight of the silk-doped fiber. In some embodiments, the base polymer content in the silk doped fiber ranges from about 10.0 wt. % to about 20.0 wt. %. In some embodiments, the base polymer content in the silk doped fiber ranges from about 20.0 wt. % to about 30.0 wt. %. In some embodiments, the base polymer content in the silk doped fiber ranges from about 30.0 wt. % to about 40.0 wt. %. In some embodiments, the base polymer content in the silk doped fiber ranges from about 40.0 wt. % to about 50.0 wt. %. In some embodiments, the base polymer content in the silk doped fiber ranges from about 50.0 wt. % to about 60.0 wt. %. In some embodiments, the base polymer content in the silk doped fiber ranges from about 60.0 wt. % to about 70.0 wt. %. In some embodiments, the base polymer content in the silk doped fiber ranges from about 70.0 wt. % to about 80.0 wt. %. In some embodiments, the base polymer content in the silk doped fiber ranges from about 80.0 wt. % to about 90.0 wt. %. In some embodiments, the base polymer content in the silk doped fiber ranges from about 90.0 wt. % to 98.0 wt. %.

In some embodiments, the silk containing therapeutic dental floss or the toothpick enhances the attachment of the therapeutic agent through physical interactions including hydrogen bonding (e.g. OH group from serine amino acid reside in the silk fibroin peptide chain forming hydrogen bond with the hydroxyl group of the saccharide repeating unit in cellulose wood fiber), ionic interactions, and hydrophobic interactions (e.g. the hydrophobic repeating peptide segments of the silk fibroin protein).

In some embodiments, the therapeutic agent is encapsulated with nanoparticles, microparticles, or microcapsules. In some embodiments, the therapeutic agents described above are encapsulated within a silk microparticles as described above, wherein the silk fibroin protein fragment forms the matrix or forms a particle shell, the therapeutic agents may be embedded with the particle matrix or enclosed inside particle shell as an oil phase or an aqueous phase (microcapsules).

### (8) Tooth Whitening Strip

In some embodiments, this disclosure provides a tooth whitening product comprising a silk film formed by casting to a substrate a tooth whitening composition having one or more dentally acceptable excipients and the silk solution or silk gel as prepared in the Example 1 and Example 9 below, wherein the tooth whitening agent applied as coating to one side of the silk film.

In some embodiments, this disclosure provides a tooth whitening product comprising a nonwoven sheet impregnated or coated with a silk tooth whitening composition having the oral care composition as described above, a tooth whitening agent, and one or more dentally acceptable excipients. In some embodiments, the silk oral care composition is a solution, a gel, a paste, a powder, an emulsion, or a suspension. In some embodiments, the silk oral care composition is a solution or a gel as described in Example 1 and Example 9 below.

In some embodiments, the application of the silk tooth whitening composition to the nonwoven sheet is by dip coating, spray coating, or immersing, or any known method for coating nonwoven fabrics.

In some embodiments, the nonwoven sheet comprises meltblown, spunbond, bonded carded, bicomponent, or crimped fibers. In some embodiments, the nonwoven sheet comprises meltblown microfibers have smaller than 10 microns in mean diameter, In some embodiments, the nonwoven sheet comprises spunbond fibers having mean diameter of about 7 μm to about 40 μm.

In some embodiments, the nonwoven sheet comprises a polymer selected from the group consisting of cellulose, rayon, nylon, polyester, polyethylene, perfluoroalkoxy alkane (PFA), polytetrafluoroethylene (PTFE), polyethylene terephthalate (Dacron®), polypropylene, polylactic acid (PLA), and combinations thereof.

In some embodiments, the one or more dentally acceptable excipients are selected from the group consisting of water, gelling agent, humectant, pH adjusting agent, desensitizing agent, stabilizing agent, bleach activator, flavoring agent, sweetener, opacifier, coloring agent, chelating agent (ethylenediaminetetraacetic acid, EDTA), and combinations thereof.

In some embodiments, the tooth whitening agent is selected from the group consisting of hydrogen peroxide, carbamide peroxide, calcium peroxide, sodium percarbonate, and combinations thereof. In some embodiments, the tooth whitening agent comprising a peroxide encapsulated within a microcapsule having a silk shell comprising the silk fibroin fragments as described above. The tooth whitening agent are highly reactive and may be difficult to keep stable for a long period of time. The silk microcapsule described herein improves the stability of the highly reactive peroxide tooth whitening agent. Further, the silk microcapsule encapsulated peroxide provides controlled release of the tooth whitening agent from the tooth whitening strip after being attached to the teeth.

In some embodiments, the silk microcapsules may be nanoparticles or microparticles. In some embodiments, the silk microcapsule has a median particle size less than 1000 nm. In some embodiments, the median particle size ranges from about 1 nm to about 1000 nm. In some embodiments, the median particle size ranges from about 1 nm to about 500 nm. In some embodiments, the median particle size ranges from about 1 nm to about 250 nm. In some embodiments, the median particle size ranges from about 1 nm to about 150 nm. In some embodiments, the median particle size ranges from about 1 nm to about 100 nm. In some embodiments, the median particle size ranges from about 1 nm to about 50 nm. In some embodiments, the median particle size ranges from about 1 nm to about 25 nm. In some embodiments, the median particle size ranges from about 1 nm to about 10 nm. In some embodiments, the silk microcapsule has a median particle size of 500 nm. In some embodiments, the silk microcapsule has a median particle size of 250 nm. In some embodiments, the silk microcapsule has a median particle size of 750 nm.

In some embodiments, the silk microcapsule has a median particle size equal or greater than 1000 nm (1 micron). In order to achieve good deposition onto skin and a stable formulation, the silk microcapsules have a median particle size ranging from about 1 μm to about 10.0 μm. In some embodiments, the silk microcapsules have a median particle size ranging from about 2 μm to about 50 μm. In some embodiments, the silk microcapsule have a median particle size ranging from about 2 μm to about 20 μm. In some embodiments, the silk microcapsules have a median particle size ranging from about 4 μm to about 10 μm. In some embodiments, the silk microcapsules have a median particle size selected from: about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.1 μm, about 2.2 μm, about 2.3 μm, about 2.4 μm, about 2.5 μm, about 2.6 μm, about 2.7 μm, about 2.8 μm, about 2.9 μm, about 3.0 μm, about 3.1 μm, about 3.2 μm, about 3.3 μm, about 3.4 μm, about 3.5 μm, about 3.6 μm, about 3.7 μm, about 3.8 μm, about 3.9 μm, about 4.0 μm, about 4.1 μm, about 4.2 μm, about 4.3 μm, about 4.4 μm, about 4.5 μm, about 4.6 μm, about 4.7 μm, about 4.8 μm, about 4.9 μm, about 5.0 μm, about 5.1 μm, about 5.2 μm, about 5.3 μm, about 5.4 μm, about 5.5 μm, about 5.6 μm, about 5.7 μm, about 5.8 μm, about 5.9 μm, about 6.0 μm, about 6.1 μm, about 6.2 μm, about 6.3 μm, about 6.4 μm, about 6.5 μm, about 6.6 μm, about 6.7 μm, about 6.8 μm, about 6.9 μm, about 7.0 μm, about 7.1 μm, about 7.2 μm, about 7.3 μm, about 7.4 μm, about 7.5 μm, about 7.6 μm, about 7.7 μm, about 7.8 μm, about 7.9 μm, about 8.0 μm, about 8.1 μm, about 8.2 μm, about 8.3 μm, about 8.4 μm, about 8.5 μm, about 8.6 μm, about 8.7 μm, about 8.8 μm, about 8.9 μm, about 9.0 μm, about 9.1 μm, about 9.2 μm, about 9.3 μm, about 9.4 μm, about 9.5 μm, about 9.6 μm, about 9.7 μm, about 9.8 μm, about 9.9 μm, and about 10.0 μm.

In some embodiments, various emulsion based particle preparation methods reported in the art such as emulsion/evaporation method may be used to prepare silk microcapsules encapsulating the tooth whitening agent. In some embodiments, the silk solution or the various silk fibroin protein fragments compositions as described above can be used to prepare the silk microcapsule encapsulated tooth whitening agent.

In some embodiments, the tooth whitening agent is present in the silk tooth whitening composition at an amount ranging from about 0.1 wt. % to about 20.0 wt. % by the total weight of the silk tooth whitening composition. In some embodiments, the tooth whitening agent is present in the silk tooth whitening composition at an amount ranging from about 0.1 wt. % to about 20.0 wt. % by the total weight of the silk tooth whitening composition. In some embodiments, the tooth whitening agent is present in the silk tooth whitening composition at an amount ranging from about 0.5 wt. % to about 15.0 wt. % by the total weight of the silk tooth whitening composition. In some embodiments, the tooth whitening agent is present in the silk tooth whitening composition at an amount ranging from about 1.0 wt. % to about 10.0 wt. % by the total weight of the silk tooth whitening composition. In some embodiments, the tooth whitening agent is present in the silk tooth whitening composition at an amount ranging from about 2.0 wt. % to about 10.0 wt. % by the total weight of the silk tooth whitening composition. In some embodiments, the tooth whitening agent is present in the silk tooth whitening composition at an amount selected from the group consisting of about 0.1 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. %, 7.0 wt. %, 8.0 wt. %, 9.0 wt. %, 10.0 wt. %, 11.0 wt. %, 12.0 wt. %, 13.0 wt. %, 14.0 wt. %, 15.0 wt. %, 16.0 wt. %, 17.0 wt. %, 18.0 wt. %, 19.0 wt. %, and 20.0 wt. % by the total weight of the silk tooth whitening composition.

The amount of tooth whitening composition provided with the tooth whitening product will vary depending upon the intended use, the size of the strip, concentration of the peroxide. In some embodiments, less about 1.0 gram of the tooth whitening composition is applied with the tooth whitening product for tooth whitening application. In some embodiments, about 0.05 gram to about 0.5 of the tooth whitening composition is applied with the tooth whitening product. In some embodiments, about 0.1 gram to about 0.4 of the tooth whitening composition is applied with the tooth whitening product.

In some embodiments, the silk whitening strip exhibits mucoadhesive and is flexibility and comfort. In addition, the silk whitening strip can circumvent the relatively short residence time of oral gels on the mucosa, which are easily washed away and removed by saliva. Moreover, in the case of local delivery for oral diseases, the silk whitening strip also help protect the wound surface, thus helping to reduce pain, and treat the disease more effectively. The silk whitening strip described here is flexible, elastic, and soft, yet adequately strong to withstand breakage due to stress from mouth movements. The silk whitening strip described here also possess good mucoadhesive strength to be retained in the mouth for the desired duration of action due to the presence of silk fibroin fragments described above.

### (9) Tooth Brushing Sheet (Tooth Wipe)

In some embodiments, this disclosure provides a disposable tooth brushing sheet comprising a nonwoven sheet impregnated or coated with the silk oral care composition described above. The brushing sheet is specially sized to comfortably fit in the user's hand to clean the mouth of an adult, a children, or an infant.

In some embodiments, the silk oral care composition is a solution, a gel, a paste, a powder, an emulsion, or a suspension. In some embodiments, the silk oral care composition is a solution or a gel. In some embodiments, the silk oral care composition comprising a fluoride. In some embodiments, the fluoride is selected from the group consisting of fluoride is selected from the group consisting of sodium fluoride, stannous fluoride, potassium fluoride, ammonium fluoride and combinations thereof.

In some embodiments, the tooth brushing sheet comprises meltblown, spunbond, bonded carded, bicomponent, or crimped fibers. In some embodiments, the tooth brushing sheet comprises meltblown microfibers have smaller than 10 microns in mean diameter, In some embodiments, the nonwoven sheet comprises spunbond fibers having mean diameter of about 7 μm to about 40 μm.

In some embodiments, the nonwoven sheet comprises a polymer selected from the group consisting of cellulose, rayon, nylon, polyester, polyethylene, perfluoroalkoxy alkane (PFA), polytetrafluoroethylene (PTFE), polyethylene terephthalate (Dacron®), polypropylene, polylactic acid (PLA), and combinations thereof. In some embodiments, the nonwoven sheet comprises an elastomeric material.

In some embodiments, the silk oral care composition comprises one or more tooth whitening agent. In some embodiments, the tooth whitening agent is selected from the group consisting of activated charcoal, silk powder and combinations thereof.

In some embodiments, the silk oral care composition comprises one or more abrasive. In some embodiments, the abrasive is silica, perlite, or bioglass.

In some embodiments, the tooth brushing sheet has a size for use in the oral cavities of human or an animals for cleaning their teeth, gums, and oral mucosa.

In some embodiments, the tooth brushing sheet may be a wet sheet. In some embodiments, the tooth brushing sheet may be a dry sheet. In some embodiments, the tooth brushing sheet may be packaged in discrete packets or stored in a bottles, or any suitable containers.

### (10) Other Oral Care Compositions

In some embodiments, this disclosure provides silk oral care compositions formulated as an irrigation fluid comprising the silk fibroin protein fragment and orally acceptable carrier as described above.

In some embodiments, this disclosure provides silk oral care compositions formulated as chewing gum compositions comprising one or more of about 50.0 wt. % to about 99.0% a gum base, about 0.4 wt. % to about 2.0 wt. % of a flavoring agent; about 0.5 wt. % to about 10.0 wt. % of silk fibroin protein fragments as described above; and about 0.01 wt. % to about 20.0 wt. % of a sweetening agent.

In some embodiments, this disclosure provides silk oral care compositions formulated as lozenges in the form of discoid-shaped solids comprising a therapeutic agent, silk fibroin protein fragments as described above in a flavored base. In some embodiments, the flavored base is selected from the group consisting of a hard sugar candy, glycerinated gelatin, a blend of sugar and mucilage, water gel of silk fibroin protein fragments derived from the silk solutions described above, hydrogel of silk fibroin protein fragments derived from the silk solutions described above, and combinations thereof.

In some embodiments, this disclosure provides silk oral care compositions formulated as dental implements impregnated with the silk oral care present composition as described above. The dental implement can be impregnated fibers including dental floss or tape, chips, or strips and polymer fibers.

### III. Silk Antiperspirant and Deodorant Products

In some embodiments, the silk personal care composition is a deodorant or antiperspirant composition and the carrier is a dermatologically acceptable medium (thereafter refers to silk deodorant or antiperspirant composition).

In some embodiments, this disclosure provides the silk antiperspirant composition comprising the silk fibroin protein fragments as disclosed above as emulsifier and a therapeutically effective amount of at least one antiperspirant active and/or at least one deodorant active. In some embodiments, the silk fibroin protein fragments in the deodorant or antiperspirant composition is a silk powder prepared by freeze-drying the silk solution as disclosed above. The silk deodorant or antiperspirant composition provides skin benefits including lubrication, conditioning by acting as emollient, miniaturization, and UV protection.

In some embodiments, the silk fibroin protein fragment in the silk deodorant or antiperspirant composition has an average weight average molecular weight selected from between about 1 kDa to about 5 kDa, about 5 kDa to about 10 kDa, about 6 kDa to about 17 kDa, about 10 kDa to about 15 kDa, about 15 kDa to about 20 kDa, b about 17 kDa to about 39 kDa, about 20 kDa to about 25 kDa, about 25 kDa to about 30 kDa, about 30 kDa to about 35 kDa, about 35 kDa to about 40 kDa, about 40 kDa to about 45 kDa, about 45 kDa to about 50 kDa, about 60 kDa to about 100 kDa, about 80 kDa to about 144 kDa, and a polydispersity of 1 to about 5.0, or about 1.5 to about 3.0.

In some embodiments, the silk deodorant or antiperspirant composition comprises about 0.5 wt. % to about 20.0 wt. % of the silk fibroin protein fragments. In some embodiments, the silk deodorant or antiperspirant composition comprises about 1.0 wt. % to about 10.0 wt. % of the silk fibroin protein fragments. In some embodiments, the silk deodorant or antiperspirant composition comprises about 1.0 wt. % to about 5.0 wt. % of the silk fibroin protein fragments. In some embodiments, the silk deodorant or antiperspirant composition comprises about 0.6 wt. % to about 2.4 wt. % of the silk fibroin protein fragments. In some embodiments, the amount of silk fibroin protein fragments is present in the silk deodorant or antiperspirant composition at an amount selected from the group consisting of about 0.5 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. %, 7.0 wt. %, 8.0 wt. %, 9.0 wt. %, 10.0 wt. %, 11.0 wt. %, 12.0 wt. %, 13.0 wt. %, 14.0 wt. %, 15.0 wt. %, 16.0 wt. %, 17.0 wt. %, 18.0 wt. %, 19.0 wt. %, and 20.0 wt. %.

The “antiperspirant active” and “deodorant active” contribute to the reduction of body malodor, for example, axillary malodor. Such reduction can be due to a masking of the malodor, absorption and/or chemical reaction of the malodorous material, reduction of the levels of the bacteria producing the malodorous materials, for example, from perspiration, reduction of perspiration, etc. The antiperspirant active materials primarily act to reduce malodor by reducing perspiration. The antiperspirant active materials can also have a deodorant function, for example, as an antimicrobial or bacteriostatic agent. The deodorant active materials do not substantially reduce perspiration, but reduce malodor in other ways. For example, as fragrances masking the malodor or reducing the malodor intensity; absorbents; antimicrobial (bacteriostatic) agents; or agents chemically reacting with malodorous materials.

In some embodiments, the deodorant active agent is selected from the group consisting of deodorant fragrances, and antimicrobial agents. In some embodiments, the antimicrobial agent is selected from the group consisting of 2-amino-2-methyl-1-propanol (AMP), cetyl-trimethylammonium bromide, cetyl pyridinium chloride, 2,4,4′-trichloro-2′-hydroxydiphenylether (triclosan), N-(4-chlorophenyl)-N′-(3,4-dichlorophenyl)urea (triclocarban), zinc ricinoleate, zinc lysine complex, ZnAlAg, ZnAlAg₃ hydrotalcites, MgAlAg₃ hydrotalcite, and combinations thereof.

In some embodiments, the antiperspirant active agent is selected from the group consisting of aluminum bromohydrate, aluminum chlorhydrates, aluminum chlorohydrex propylene glycol, aluminum dichlorohydrex propylene glycol, aluminum sesquichlorohydrex propylene glycol, aluminum chlorohydrex polyethylene glycol, aluminum dichlorohydrex polyethylene glycol, aluminum sesquichlorohydrex polyethylene glycol, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octachlorohydrate, aluminum zirconium trichlorohydrex gly, aluminum zirconium tetrachlorohydrex gly, aluminum zirconium pentachlorohydrex gly, aluminum zirconium octachlorohydrex gly, buffered aluminum sulfate, potassium alum, sodium aluminum chlorohydroxy lactate, aluminum sesquichlorohydrates, sodium aluminum lactate, or mixtures thereof.

In some embodiments, the silk antiperspirant composition may further comprise a solvent for the antiperspirant active. In some embodiments, the solvent is selected from the group consisting of water, propylene glycol, dipropylene glycol, tripropylene glycol butylene glycol, 1,2-hexanediol, dimethyl isosorbide, polyhydric alcohols having 3-9 carbons, polymeric ethers having 5-30 units of ethylene oxide or propylene oxide.

In some embodiments, the silk antiperspirant composition comprises about 0.1 wt. % to about 25.0 wt. % of the antiperspirant active. In some embodiments, the silk antiperspirant composition about 5.0 wt. % to about 25.0 wt. % of the antiperspirant active. In some embodiments, the silk antiperspirant composition about 15.0 wt. % to about 25.0 wt. % of the antiperspirant active. When the amount of the antiperspirant active is presented in an amount ranging from about 0.1 wt. % to about 10.0 wt. % by the total weight of the silk antiperspirant composition, the antiperspirant active material will not substantially reduce the flow of perspiration, but will reduce malodor, for example, by acting as an antimicrobial material.

In some embodiments, the silk antiperspirant composition comprises silk fibroin protein hydrogel microparticle encapsulated antiperspirant active agent. The silk fibroin protein hydrogel microparticle is prepared by the following steps: (1) providing the silk solution as described above, (2) sonicating the silk solution, optionally vitamin C is added to the silk solution; (3) adding the antiperspirant active agent to the silk solution of step 2 just after sonication and the solution is mixed by inversion, in which the sol-gel transition is initiated, but the silk fibroin protein fragments are still in the solution state; (4) immediately after mixing, the antiperspirant active in silk solution mixture of step (3) is added dropwise to a sunflower oil bath in a petri dish and then incubating at ambient condition overnight to allow complete sol-gel transition; (5) removing silk hydrogel microparticle encapsulated antiperspirant active agent from the oil. The emulsion of sunflower oil in silk is prepared with silk concentration at about 0.6% (w/v), 1.2% (w/v), 2.4% (w/v), 4.0% (w/v), and 6.0% (w/v), and volume ratio of oil to the silk solution is at 4:1, 2:1, 3:2, 1:1, 2:3, 1:2, and 1:4. The oils suitable for making the silk hydrogel microparticle may include squalane, jojoba oil, and combinations thereof.

In some embodiments, the silk fibroin solution used to encapsulate the antiperspirant active agent comprises silk fibroin protein fragments having an average weight average molecular weight selected from between about 40 kDa to about 45 kDa, about 45 kDa to about 50 kDa, about 60 kDa to about 100 kDa, about 80 kDa to about 144 kDa, about 135 kDa to about 140 kDa, about 145 Da to about 150 Da, about 150 kDa to about 155 kDa, about 155 kDa to about 160 kDa, about 160 kDa to about 165 kDa, about 165 kDa to about 170 kDa, about 170 kDa to about 175 kDa, about 175 kDa to about 180 kDa, about 180 kDa to about 185 kDa, about 185 kDa to about 190 kDa, about 190 kDa to about 195 kDa, about 195 kDa to about 200 kDa, about 200 kDa to about 205 kDa, about 205 kDa to about 210 kDa, about 210 kDa to about 215 kDa, about 215 kDa to about 220 kDa, about 220 kDa to about 225 kDa, about 225 kDa to about 230 kDa, about 230 kDa to about 235 kDa, about 235 kDa to about 240 kDa, about 245 kDa to about 250 kDa, about 250 kDa to about 255 kDa, about 255 kDa to about 260 kDa, about 260 kDa to about 265 kDa, about 265 kDa to about 270 kDa, about 270 kDa to about 275 kDa, about 275 kDa to about 280 kDa, about 285 kDa to about 290 kDa, about 290 kDa to about 295 kDa, about 295 kDa to about 300 kDa, about 300 kDa to about 305 kDa, about 305 kDa to about 310 kDa, about 310 kDa to about 315 kDa, about 315 kDa to about 320 kDa, about 320 kDa to about 325 kDa, about 325 kDa to about 330 kDa, about 330 kDa to about 335 kDa, about 335 kDa to about 340 kDa, about 340 kDa to about 345 kDa, about 345 kDa to about 350 kDa, and a polydispersity of 1 to about 5.0, or about 1.5 to about 3.0.

In some embodiments, the silk hydrogel microparticle encapsulated antiperspirant active having a median particle size of about 1 μm to about 75 μm. In some embodiments, the silk hydrogel microparticle encapsulated antiperspirant active having a median particle size of 1 μm to about 10 μm. In some embodiments, the silk hydrogel microparticle encapsulated antiperspirant active having a median particle size selected from the group consisting of about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, and about 75 μm.

In some embodiments, the antiperspirant active agent protected by the silk hydrogel microparticle comprises about 1.0 wt. to about 50.0 wt. % encapsulated antiperspirant active salt, and about 10.0 wt. % to about 70.0 wt. % silk fibroin protein fragments by the total weight of the silk hydrogel microparticle.

In some embodiments, the silk antiperspirant composition contains a cosmetically effective amount of the silk hydrogel microparticle encapsulated antiperspirant active ranging from about 0.01 wt. % to about 40.0 wt. % by the total weight of the silk antiperspirant composition. In some embodiments, the silk antiperspirant composition contains a cosmetically effective amount of the silk hydrogel microparticle encapsulated antiperspirant active ranging from about 1.0 wt. % to about 30.0 wt. % by the total weight of the silk antiperspirant composition. In some embodiments, the silk antiperspirant composition contains a cosmetically effective amount of the silk hydrogel microparticle encapsulated antiperspirant active ranging from about 5.0 wt. % to about 20.0 wt. % by the total weight of the silk antiperspirant composition.

The antiperspirant composition having the silk hydrogel microparticle encapsulated antiperspirant active agent as described herein mitigates the skin irritation issue caused by the antiperspirant salts in sensitive individuals. Since the internal constituents of silk hydrogel microparticles are insulated from the surrounding environment (i.e. skin), and released only when needed, skin irritation may be substantially alleviated in susceptible individuals. Microencapsulation of antiperspirant salts also provides for controlled release of the internal constituents, hence lengthening the effective period of the antiperspirant.

In some embodiments, the silk antiperspirant composition further comprises a gelling agent. In some embodiments, the gelling agent is selected from the group consisting of vitamin C, 12-hydroxystearic acid, esters of 12-hydroxystearic acid, amides of 12-hydroxystearic acid, N-lauroyl-L-glutamic acid dibutylamide, N-2-ethylhexanoyl-L-glutamic acid dibutylamide, and combinations thereof. In some embodiments, the gelling agent for the silk antiperspirant composition is vitamin C. In some embodiments, the gelling agent for the silk antiperspirant composition is selected from the group consisting of N-lauroyl-L-glutamic acid dibutylamide, N-2-ethylhexanoyl-L-glutamic acid dibutylamide, and combinations thereof.

In some embodiments, the silk antiperspirant composition comprises about 1.0 wt. % to about 15.0 wt. % of the gelling agent. In some embodiments, the silk antiperspirant composition comprises about 3.0 wt. % to about 12.0 wt. % of the gelling agent. In some embodiments, the silk antiperspirant composition comprises about 5.0 wt. % to about 10.0 wt. % of the gelling agent.

In some embodiments, the silk deodorant or antiperspirant compositions are prepared with the emulsifiable oily components as described above to form emulsion or suspension stick products. The emulsion stick antiperspirant is characteristically having an internal phase and an external phase. The external phase is defined as the continuous phase where liquids are interconnected. The internal phase is defined as the suspended phase where liquids exist in a droplet form stabilized by surfactants. In some embodiments, the emulsion stick antiperspirant forms gelled antiperspirant compositions having a gelled oil phase as the external phase and an internal phase containing the antiperspirant active agent. The external gelled oil phase contains silk fibroin protein fragment emulsifier or silk powder, oil, emollient, and gelling agent, as well as optional additives for the antiperspirant product such as surfactants, fragrances, additional emollients etc. The internal phase consists of a liquid solution containing silk hydrogel encapsulated antiperspirant active agent, or dissolved antiperspirant active agent without encapsulation, and solvents including water, propylene glycol, dipropylene glycol, tripropylene glycol, ethanol, and 1,2-hexanediol.

The oil used for the gelled oil phase in the silk antiperspirant composition described herein is not particularly limited so long as the oil sufficiently dissolves the gelling agent by heating and forms a gel when cooled to room temperature. In some embodiments, the oil is selected from the group consisting of silicone oil, cetyl alcohol, isostearyl alcohol, lauryl alcohol, hexadecyl alcohol, octyldodecanol, isostearic acid, undecylenic acid, oleic acid, myristyl myristate, hexyl laurate, decyl oleate, isopropyl myristate, hexyldecyl dimethyloctanoate, glyceryl monostearate, diethyl phthalate, ethylene glycol monostearate and octyl oxystearate, liquid paraffin, isoparaffin, vaseline, squalane, lanolin, reduced lanolin, carnauba wax, mink oil, cacao oil, coconut oil, palm seed oil, camellia oil, sesame oil, castor oil and olive oil, polyethylene/α-olefin wax, and combinations thereof.

In some embodiments, the silicone oil is selected from the group consisting of methylpolysiloxane, highly polymerized methylpolysiloxane, polyoxyethylene/methylpolysiloxane copolymer, polyoxypropylene/methylpolysiloxane copolymer and poly(oxyethylene or oxypropylene)/methylpolysiloxane copolymer, stearoxymethylpolysiloxane, stearoxytrimethylsilane, methyl hydrogen polysiloxane, octamethylpolysiloxane, decamethylpolysiloxane, decamethylcyclopentasiloxane, octamethylcyclotetrasiloxane, tetrahydrotetramethylcyclotetrasiloxane, methylcyclopolysiloxane, cyclopentasiloxane, dodecamethylcyclohexasiloxane, methylphenylpolysiloxane, trimethylsiloxy silicate, aminoethylaminopropylsiloxane/dimethylsiloxane copolymer, silanol-modified polysiloxanes, alkoxy-modified polysiloxanes, aliphatic acid-modified polysiloxanes, fluorine-modified polysiloxanes, epoxy-modified polysiloxanes, alkoxy-modified polysiloxane perfluoropolyethers, polyvinyl acetate dimethyl polysiloxane, and combinations thereof.

In some embodiments, the oil is presented in the gelled silk antiperspirant composition at an amount ranging from about 10.0 wt. % to about 99.9 wt. % by the total weight of the gelled antiperspirant composition. The amount of the oil is less than 10.0 wt. % or more than 99.9 wt. % by the total weight of the silk deodorant or antiperspirant composition.

In some embodiments, the silk deodorant or antiperspirant composition further comprises one or more additional additives selected from the group consisting of rhamnolipid, aloe vera, a humectant, a moisturizer, an astringent, an antiseptic agent, a gellant, a surfactant, a thickening agent, a cosmetic powder, a fragrance, a sunscreen, an antimicrobial, a preservative, a coloring agent, a pigment, a filler, a co-emulsifier, a hardener, a strengthener, a chelating agent, a thixotropic agent, an oil absorbing agent, an antioxidant, an amino acid, a polyhydric alcohol, a polyamino acid, a water-soluble polymer, a sugar alcohol, a lower alcohol, an animal extract, a plant extract, a nucleic acid, an organic fine particle, an inorganic fine particle, a pH adjusting agent, a pearling agent, a wetting agent, and combinations thereof.

In some embodiments, the deodorant or antiperspirant composition is formulated as a product selected from the group consisting of a stick, a roll-on, a powder, a gel, an aerosol, a paste, and a cream. In some embodiments, the deodorant or antiperspirant composition has clear, transparent, or translucent appearance.

The silk deodorant or antiperspirant products containing silk hydrogel encapsulated antiperspirant active agent may provide advantage of increased wear resistance due to the high affinity of silk protein to the skin resulted from the presence of hydrophilic amino acid residue in the silk fibroin protein, for example, —OH group from serine, guanidine group from arginine, free amine group from lysine, —COOH group from aspartic acid and glutamic acid of the silk fibroin protein.

### IV. Silk Nail Care Products

The conventional nitrocellulose containing nail polish formulations present a number of challenges. For example, nitrocellulose discolors with age, is prone to undergo sharp viscosity changes rendering nail care compositions difficult to apply, and it can be difficult to dry to a hard film. Furthermore, care must be taken to ensure that nitrocellulose used in formulating nail care composition is neutral, i.e., acid free, because the presence of free acid could cause damage to fingernails and the cuticle, as well as have a deleterious effect on colorants present in nail care compositions. Moreover, there is additional difficulty caused by potential sedimentation of pigments and/or pearlescent agents in the composition to maintain its homogeneity in the packaging container.

There is a need for substitutes of nitrocellulose as a film former for nail care compositions. Attempts to find substitutes for nitrocellulose have not been successful, because, despite its many drawbacks, nitrocellulose provides nail care compositions with an unusual combination of desirable properties such as toughness, durability and solvent release, and it produces waterproof and atmospherically stable films. Typical nitrocellulose containing nail lacquer compositions are described in U.S. Pat. Nos. 4,097,589 and 4,179,304.

There is, therefore, a long felt need in the art for a film-forming agent that can be substituted for nitrocellulose to give finished nail care compositions that possess the same use qualities provided by the nitrocellulose-based formulations.

In some embodiments, the silk personal care composition is a nail care composition and the carrier is a dermatologically acceptable medium (silk nail care composition). In some embodiments, the silk fibroin protein fragments as described herein are further modified to form alkyl ester derivatives of the silk fibroin protein fragments, for example, ethyl ester of the silk fibroin protein fragments. In some embodiments, the nail care composition comprises the ethyl ester of the silk fibroin protein fragments and an alcohol to give nail remover, or nail polish composition. The silk fibroin protein fragments and ethyl ester of the silk fibroin protein fragments have good film forming property.

In some embodiments, the nail care composition further comprises an additive selected from the group consisting of rhamnolipid, a film-forming agent, a suspending agent, a thixotropic agent, a coloring agent, a pigment, a glitter, a plasticizer, a thickening agent, a nail hydrating agent, a nail hardening agent, boric acid, a vitamin, a plant extract, an essential oil, a preservative, a mineral salt, a fruit extract, an algae extract, a fungus extract, a caviar extract, an aldehydes, a vegetable oil, an amino acid, a peptide, a protein, a ceramide, allantoin or an allantoin derivative, an organosilicon derivative, an antioxidant, a UV light absorber, a moisturizer, a stabilizer, a fragrance, a micronutrient, a dye, a pigment, and combinations thereof. In some embodiments, the nail care composition is formulated as a nail polish, or nail polish remover. The silk fibrin protein fragment film exhibits high gloss.

In some embodiments, additional film forming polymer suitable for the silk nail care composition is selected from the group consisting of aryl sulfonamide/formaldehyde, sucrose acetate isobutyrate, sucrose benzoate, diethylene/dipropylene glycol dibenzoate, and combinations thereof.

In some embodiments, the silk nail care compositions further comprises at least one co-film forming agent selected from the group consisting of tosylamide epoxy resins such as those sold under the Polytex name by Estron Chemical, Inc. (for example, E-75, E-100 and NX-55), polyvinyl butyral, acrylic (co)polymers, acrylic resins, styrene resins, acrylate-styrene resins, vinyl resins, vinyl copolymers, polyurethanes, polyesters, alkyd resins, cellulose polymers, nitrocellulose, cellulose esters, cellulose acetate, cellulose acetate propionate, cellulose acetate butyrate, resins resulting from the condensation of formaldehyde with an arylsulphonamide, starches and derivatives thereof, natural or synthetic gums and derivatives thereof, water soluble adhesives and combinations thereof. In some embodiments, the silk nail care compositions further comprises at least one co-film forming agent selected from the group consisting of polyester, acrylic resin, and combinations thereof.

In some embodiments, useful (meth)acrylic polymers or acrylic resins include, but are not limited to, copolymers of methyl methacrylate with butyl acrylate, butyl methacrylate, isobutyl methacrylate, or isobornyl methacrylate (e.g., PARALOID™ DM-55, PARALOID B48N, PARALOID™ B66, ELVACITE™ 2550), copolymers of isobutylmethacrylate and butyl methacrylate (e.g., ELVACITE™ 2046), and isobutyl methacrylate polymers (e.g., PARALOID™ B67).

In some embodiments, useful polyester resins include, but are not limited to, polyester resins formed by reacting a polyhydric alcohol with a polybasic acid (e.g., a polyester resin obtained by reacting trimellitic acid, neopentyl glycol, and adipic acid, sold under the trade name UNIPLEX™ 670-P, Unitex Chemical Corporation).

In some embodiments, the silk nail care composition comprises about 0.1 wt. % to about 50.0 wt. % of at least one epoxy resin co-film forming agent. In some embodiments, the silk nail care composition comprises about 1.0 wt. % to about 40.0 wt. % of at least one epoxy resin co-film forming agent. In some embodiments, the silk nail care composition comprises about 10.0 wt. % to about 30.0 wt. % of at least one epoxy resin co-film forming agent.

In an embodiment, this disclosure provides a silk nail polish composition, comprising (a) silk fibroin protein fragments having an average weight average molecular weight selected from between about 1 kDa and about 5 kDa, between about 5 kDa and about 10 kDa, between about 6 kDa and about 17 kDa, between about 10 kDa and about 15 kDa, between about 15 kDa and about 20 kDa, between about 17 kDa and about 39 kDa, between about 20 kDa and about 25 kDa, between about 25 kDa and about 30 kDa, between about 30 kDa and about 35 kDa, between about 35 kDa and about 40 kDa, between about 39 kDa and about 80 kDa, between about 40 kDa and about 45 kDa, between about 45 kDa and about 50 kDa, between about 60 kDa and about 100 kDa, and between about 80 kDa and about 144 kDa, and a polydispersity between 1 and about 5; 0 to 500 ppm lithium bromide; (b) at least one co-film forming agent chosen from an epoxy resin; (c) at least one solvent chosen from at least one volatile solvent and water; (d) optionally, at least one plasticizer; and (e) optionally, at least one colorant, wherein the nail care composition does not require the use of nitrocellulose.

In some embodiment, the silk nail care compositions provide a degree of gloss that is at least comparable, and oftentimes higher, than that of conventional nitrocellulose-containing composition.

In some embodiment, the silk nail care composition further comprises at least one plasticizer. Any plasticizing agent typically found in nail polish compositions can be used. In some embodiments, suitable plasticizing agent is selected from the group consisting of tributyl phosphate, tributoxyethyl phosphate, tricresyl phosphate, triphenyl phosphate, glycerol triacetate, butyl stearate, butyl glycolate, benzyl benzoate, butyl acetyltricinoleate, glyceryl acetyltricinoleate, dibutyl phthalate, diisobutyl phthalate, dioctyl phthalate, dimethoxyethyl phthalate, diamyl phthalate, triethyl citrate, tributyl citrate, tributyl acetylcitrate, tri(2-ethylhexyl) acetylcitrate, dibutyl tartrate, camphor, isopropyl alcohol fatty acid ester, C₈ alcohol fatty acid ester, organic succinate, organic phthalate, and combinations thereof.

In some embodiment, the silk nail care composition comprises about 0.01 wt. % to about 25.0 wt. % of the plasticizing agent. In some embodiment, the silk nail care composition comprises about 0.1 wt. % to about 22.0 wt. % of the plasticizing agent. In some embodiment, the silk nail care composition comprises about 1.0 wt. % to about 20.0 wt. % of the plasticizing agent.

In some embodiments, the silk nail care composition comprises a solvent. Any solvent typically found in nail polish compositions can be used. In some embodiments, suitable solvent is selected from the group consisting of ketones including methyl ethyl ketone, methyl isobutyl ketone, diisobutyl ketone, isophorone, cyclohexanone or acetone; alcohols including ethanol, isopropanol, diacetone alcohol, 2-butoxyethanol or cyclohexanol; polyhydric alcohol including ethylene glycol, propylene glycol, pentylene glycol or glycerol; propylene glycol ether including propylene glycol monomethyl ether, propylene glycol monomethyl ether acetate or dipropylene glycol mono(n-butyl)ether; short-chain ester (having a total of 2 to 7 carbon atoms) including ethyl acetate, methyl acetate, propyl acetate, n-butyl acetate or isopentyl acetate; hydrocarbon including decane, heptane, dodecane or cyclohexane; aldehyde including benzaldehyde, acetaldehyde; water, and combinations thereof. In some embodiments, suitable solvent comprises short-chain esters (having a total of from 2 to 8 carbon atoms).

In some embodiments, the silk nail care composition comprises about 1% to about 90% by weight, preferably from about 10.0 wt. % to about 80.0 wt. % of the solvent. The silk nail care composition comprises about 30.0 wt. % to about 75.0 wt. % of the solvent.

In some embodiments, the silk nail care composition further comprises a coloring agent as described above to impart color to the nails.

In some embodiments, the silk nail care composition further comprises at least one pearlescent pigment selected from the group consisting of white pearlescent pigment, mica coated with titanium oxide, mica coated with bismuth oxychloride, colored pearlescent pigment, titanium oxide-coated mica with iron oxides, titanium oxide-coated mica with ferric blue, titanium oxide-coated mica with chromium oxide, titanium oxide-coated mica with an organic pigment, and pearlescent pigments based on bismuth oxychloride, and combinations thereof.

In some embodiments, the silk nail care composition comprises about 0.01 wt. % to about 20.0 wt. % of the coloring agent. In some embodiments, the silk nail care composition comprises about 0.1 wt. % to about 15.0 wt. % of the coloring agent. In some embodiments, the silk nail care composition comprises about 0.5 wt. % to about 10.0 wt. % of the coloring agent.

In some embodiments, the silk nail care composition further comprises an additive selected from the group consisting of thickener, coalescent, preservative, fragrance, oil, wax, surfactant, antioxidant, free radical scavenger, spreading agent, wetting agent, dispersing agent, antifoaming agent, pH adjusting agent, stabilizing agent, essential oil, sunscreen, moisturizing agent, vitamin, proteins ceramide, plant extract, fiber, and combinations thereof.

In some embodiments, the nail care composition comprises about 99.0 wt. % or less of the additive. In some embodiments, the silk nail care composition comprises about 0.01 wt. % to about 90.0 wt. % of the additive. In some embodiments, the nail care composition comprises about 0.1 wt. % to about 50.0 wt. % of the additive.

### V. Silk Skin Care and Makeup Products

The structure and the contents of the amino acids in silk fibroin proteins are akin to human skin. Thus, silk fibroin fragment as described herein has high affinity to the skin that any other natural protein cannot have. Both the silk fibroin protein and skin can block ultraviolet rays of the sun, to prevent skin from burning by sun light. The silk powder has unique ability to trap oil, and has high affinity to the fatty components of the skin corneum.

In some embodiments, this disclosure provide a silk skin care composition comprising silk fibroin protein fragments as described above which causes no irritation to the skin, has excellent properties such as moisture retention, adhesion to the skin, durability of the makeup, fragrance retention and the like, and can give an agreeable feeling and a natural silk-like gloss to the skin.

In some embodiments, the silk skin care composition is formulated as a cream, an emulsion, a shaving or after-shave cream, a foam, a conditioner, a cologne, a shaving or after-shave lotion, a perfume, a cosmetic oil, a facial mask, a moisturizer, an anti-wrinkle, an eye treatment, a tanning cream, a tanning lotion, a tanning emulsion, a sunscreen cream, a sunscreen lotion, a sunscreen emulsion, a tanning oil, a sunscreen oil, a hand lotion, a body lotion, a color cosmetic, a mascara, a lipstick, a lip liner, an eye shadow, an eye-liner, a rouge, a face powder, a foundation, a blush, and perfume.

### A. Makeup Composition

Conventional makeup compositions in powder form (e.g., face powder and the like) consist mainly of titanium dioxide, zinc white, talc, metallic soaps and precipitated calcium carbonate. However, such compositions cannot be formulated easily because slight variations in composition may cause changes in covering power, lubricity, absorbing power, water resistance and the like. It is well known that some properties (such as adhesion to the skin, spreadability on the skin, feeling, finish of the makeup, and the like) of such makeup compositions in powder form can be improved by addition of a nonionic surface-active agent having low HLB, such as polyoxyethylene glycol diesters, esters and ethers of polyhydric alcohols to the base materials. However, the addition of such a surface-active agent is disadvantageous in that it causes irritation to the skin.

In some embodiments, this disclosure provides a makeup composition comprising the lyophilized silk powders derived from freeze-drying the silk solution as described above. The silk powders in the powder makeup composition impart silky touch, moisturizing touch on skin, moderate hydrophilicity, lipophilicity, good adhesion to skin. The fiber structure of the silk fibroin can prevent the powder makeup composition from aggregating, hardening or caking.

In some embodiments, the silk makeup composition further comprises a cosmetic ingredient selected from the group consisting of a skin conditioning agent, an oil control agent, an anti-acne agent, an astringent, a skin calming agent, a plant extract, an essential oil, a humectant, a moisturizer, a structurant, a gelling agent, an antioxidant, an anti-aging compound, a sunscreen, a skin lightening agent, a sequestering agent, a preserving agent, a filler, a fragrance, a thickener, a wetting agent, a dye, a pigment, a cosmetic powder, and combinations thereof.

In some embodiments, the silk makeup composition further comprises pigment particles coated with a film of silk fibroin protein fragments formed by coating pigment particles with the silk solution disclosed above.

In some embodiments, the amount of silk fibroin protein film coating is present in an amount of from 2.0 wt. % to 100 wt. % by the total weight of the uncoated pigment. In some embodiments, the amount of silk fibroin protein film coating is present in an amount of from 5.0 wt. % to 50 wt. % by the total weight of the uncoated pigment. If the amount is less than 2.0 wt. % by weight, it is difficult to impart the fibroin-coated pigment with properties such as adhesion to the skin, spreadability on the skin, dispersibility, covering power, skin-protecting ability, feeling and the like.

In some embodiments, the uncoated pigment particle is selected from the group consisting of white pigments, color pigments, extender pigments, pearlescent pigments, talc, kaolin, mica, calcium carbonate, titanium oxide, zinc oxide, micaceous titanium, magnesium carbonate, yellow iron oxide, red iron oxide, black iron oxide, ultramarine, zinc stearate, magnesium stearate, magnesium silicate, organic pigment, and combinations thereof.

In some embodiments, the uncoated pigment particle has a median diameter ranging from 0.05 μm to 100 μm. In some embodiments, the uncoated pigment particle has a median diameter ranging from 0.05 μm to 60 μm. In some embodiments, the uncoated pigment particle has a median diameter ranging from 0.1 μm to 30 μm.

In some embodiments, the thickness of the silk fibroin protein fragment film coating layer ranges from 0.01 μm to 50 μm. The coating film of silk fibroin protein fragments has an average weight average molecular weight of 40 kDa or greater. In some embodiments, the coating film of silk fibroin protein fragments has an average weight average molecular weight selected from between about 60 kDa and about 100 kDa, or from between about 80 kDa and about 144 kDa, and a polydispersity of 1 to about 5.0, or about 1.5 to about 3.0.

In some embodiments, the silk fibroin protein fragments-coated pigment is presented in the makeup composition at an amount ranging from about 10.0 wt. % to about 90.0 wt. % by the total weight of the makeup composition. In some embodiments, the silk fibroin protein fragments-coated pigment is presented in the makeup composition at an amount ranging from about 20.0 wt. % to about 80.0% by weight based on the total weight of the silk makeup composition.

In some embodiments, the silk makeup composition further comprises an additional powder component selected from the group consisting of clay mineral powders such as talc, mica, sericite, silica, magnesium silicate, synthetic fluorophlogopite, calcium silicate, aluminum silicate, bentonite, montmorillonite; pearl powders such as alumina, barium sulfate, calcium secondary phosphate, calcium carbonate, titanium oxide, zirconium oxide, zinc oxide, hydroxy apatite, iron oxide, iron titanate, ultramarine blue, Prussian blue, chromium oxide, chromium hydroxide, cobalt oxide, cobalt titanate, titanium oxide coated mica; organic powders such as polyester, polyethylene, polystyrene, methyl methacrylate resin, cellulose, 12-nylon, 6-nylon, styrene-acrylic acid copolymers, polypropylene, vinyl chloride polymer, tetrafluoroethylene polymer, boron nitride, fish scale guanine, laked tar color dyes, laked natural color dyes, spherical alumina, polyacrylates, silicates, sulfates, metal dioxides, carbonates, celluloses, polyalkylenes, vinyl acetates, polystyrenes, polyamides, acrylic acid ethers, silicones, and combinations thereof.

In some embodiments, the silk makeup composition further comprises an oil absorbing powder selected from the group consisting of silica, silicate salts, carbonate salts, metal oxides, and hydroxyapatite, methyl methacrylate copolymers, and combinations thereof.

In some embodiments, the silk makeup composition containing the silk fibroin protein fragments coated pigment particles are formed into cakes. In some embodiments, the makeup composition containing the silk fibroin protein fragments coated pigment particles is formulated as a product selected from the group consisting of a color cosmetic, a lip liner, an eye shadow, an eye-liner, a rouge, a face powder, a foundation, and a blush.

In some embodiments, the silk makeup composition has excellent use properties including moisture retention, fragrance retention, affinity for the skin, spreadability on the skin, feeling, silk-like gloss, durability of the makeup, and causing no irritation to the skin.

Mascara is a commonly used cosmetic applied to the eyelashes to enhance their appearance. An effective mascara composition must appear to thicken and lengthen the lashes, must be easy to apply and remove, must apply evenly, not smudge or flake, and be long lasting and water resistant after application. It also must be readily removable with conventional eye makeup removers, must not cause allergic reactions, and must be safe for contact lens wearers.

Although a great number of mascara compositions exist, there is a need for a mascara composition that is long lasting, easy to apply, water resistant, safe, and effective in thickening and lengthening the lashes and effective in imparting an attractive appearance to the lashes of the wearer. There is also a need for a mascara composition that provides care for and improves the actual condition of the eyelashes by making them softer, smoother, and more pliable.

In some embodiments, this disclosure provides a silk mascara comprising the silk fibroin protein fragments as described above. In some embodiments, the silk fibroin protein fragment has an average weight average molecular weight selected from between about 10 kDa to about 15 kDa, about 15 kDa to about 20 kDa, b about 17 kDa to about 39 kDa, about 20 kDa to about 25 kDa, about 25 kDa to about 30 kDa, about 30 kDa to about 35 kDa, about 35 kDa to about 40 kDa, about 40 kDa to about 45 kDa, about 45 kDa to about 50 kDa, and a polydispersity of 1 to about 5.0, or about 1.5 to about 3.0.

In some embodiments, the silk mascara composition further comprises a wax selected from the group consisting of rose wax and jasmine wax. In some embodiments, the wax is presented in the mascara composition at an amount ranging from about 0.1 wt. % to about 3.0 wt. % by the total weight of the silk mascara composition.

In some embodiments, the silk mascara further comprises a vitamin selected from the group consisting of ascorbyl palmitate, ascorbyl myristate, ascorbyl stearate, tocopheryl acetate, tocopheryl propionate, tocopheryl butyrate, panthenol, and combinations thereof. In some embodiments, the silk mascara further comprises a vitamin selected from the group consisting of ascorbyl palmitate, tocopheryl acetate, and panthenol. In some embodiments, the ascorbyl palmirate is presented in the mascara composition at an amount ranging from about 0.05 wt. % to about 0.25 wt. % by the total weight of the mascara composition. In some embodiments, the tocopheryl acetate is presented in the mascara composition at an amount ranging from about 0.1 wt. % to about 0.3 wt. % by the total weight of the mascara composition. In some embodiments, the panthenol is presented in the mascara composition at an amount ranging from 0.01 wt. % to about 0.25 wt. % by the total weight of the mascara composition.

In some embodiments, the silk fibroin protein fragment is presented in the silk mascara composition at an amount ranging from about 0.5 wt. % to about 5.0 wt. % by the total weight of the silk mascara composition. In some embodiments, the silk fibroin protein fragment is presented in the silk mascara composition at an amount ranging from about 0.6 wt. % to about 2.4 wt. % by the total weight of the silk mascara composition.

In some embodiments, the silk mascara composition further comprises a cosmetic additive selected from the group consisting of an antioxidant, a preservative, a synthetic emulsifier, a solvent, a thickener, a colorant, and combinations thereof.

In some embodiments, the silk mascara composition possesses the properties including longer lasting water resistant, easy to apply, and effective in lengthening and thickening the eyelashes of the wearer. It is safe for contact lens wearers and readily removable with conventional eye makeup.

In some embodiments, this disclosure provides a makeup composition formulated as a silk lipstick comprising the silk fibroin protein fragments as described above. In some embodiments, the silk fibroin protein fragments in the silk lipstick has an average weight average molecular weight selected from between about 10 kDa to about 15 kDa, about 15 kDa to about 20 kDa, b about 17 kDa to about 39 kDa, about 20 kDa to about 25 kDa, about 25 kDa to about 30 kDa, about 30 kDa to about 35 kDa, about 35 kDa to about 40 kDa, about 40 kDa to about 45 kDa, about 45 kDa to about 50 kDa, and a polydispersity of 1 to about 5.0, or about 1.5 to about 3.0. In some embodiments, the silk fibroin protein fragment is presented in the silk lipstick at an amount ranging from about 0.6 wt. % to about 3.0 wt. % by the total weight of the silk lipstick. In some embodiments, the silk fibroin protein fragment is presented in the lipstick at an amount ranging from about 0.6 wt. % to about 2.4 wt. % by the total weight of the silk lipstick.

In some embodiments, the silk lipstick further comprises an additional ingredient selected from the group consisting of Portulaca pilosa extract, sunflower oil, jojoba esters, mango butter, tocopherol, orange peel wax, Limnanthes alba seed oil, Butyrospermum parkii, ethyl macadamiate, sucrose acetate isobutyrate, sunflower wax, candelilla wax, beeswax, titanium dioxide, mica silk, castor oil, buriti oil, and combinations thereof.

### B. Skin Care Composition

In some embodiments, the silk personal care composition is a silk skin care composition and the carrier is a dermatologically acceptable medium. In some embodiments, the skin care composition further comprises a skin conditioning agent selected from the group consisting of amino acids derived from silk fibroin protein, silicone skin conditioning agent, mineral oil, emu oil, plant extract, cell extract, rice extract, rice flour, oat flour, aloe vera, protease, salmon zonase, microbial exopolysaccharide, algae derived polysaccharide, and combinations thereof.

In some embodiments, the silk skin care composition further comprises a cosmetic ingredient selected from the group consisting of a surfactant (e.g., sophorolipid), a skin conditioning agent, an oil control agent, an anti-acne agent, an astringent, a scrub particle or agent, an exfoliating particle or agent, a skin calming agent, a plant extract, an essential oil, a coolant, a humectant, a moisturizer, a structurant, a gelling agent, an antioxidant, an anti-aging compound, a sunscreen, a skin lightening agent, a sequestering agent, a preserving agent, a filler, a fragrance, a thickener, a wetting agent, a dye, a pigment, a glitter, and combinations thereof.

In some embodiments, the silk skin care composition is formulated as a product selected from the group consisting of a cream, an emulsion, a shaving or after-shave cream, a foam, a conditioner, a cologne, a shaving or after-shave lotion, a perfume, a cosmetic oil, a facial mask, a moisturizer, an anti-wrinkle, an eye treatment, a tanning cream, a tanning lotion, a tanning emulsion, a sunscreen cream, a sunscreen lotion, a sunscreen emulsion, a tanning oil, a sunscreen oil, a hand lotion, and a body lotion.

In some embodiments, this disclosure provides a skin care product comprising the silk skin care composition or the silk fibroin protein fragment composition described above and a substrate, wherein the substrate is impregnated with the skin care composition or the silk fibroin protein fragment composition. In some embodiments, the substrate comprising natural fibers derived from a plant selected from the group consisting of flax, hemp, jute, ramie, nettle, Spanish broom, kenaf plants, and combinations thereof. In some embodiments, additional synthetic fibrous material suitable for use as the substrate is selected from the group consisting of melt-blown polyethylene, polypropylene, copolymers of polyethylene and polypropylene, fibers formed of diblock copolymers of polyethylene and polypropylene, and combinations thereof. In some embodiments, the substrate also may be embossed.

In some embodiments, the substrate is selected from the group consisting of a web, a gauze, a cotton swab, a nonwoven, a wipe, a transdermal patch, a pad, flushable or nonflushable web of cellulosic fibers, a web of synthetic fibrous material, tissue, towel or napkin, and a hydrogel.

In some embodiments, the substrate comprises hydrogel. In some embodiments, the substrate is a non-woven fabric or tissue comprising natural fiber selected form cellulose fiber, regenerated cellulose fiber, wood pulp fibers. In some embodiments, the substrate is selected from the group consisting of a wet wipe, a dry wipe, impregnated wipe, a wet pad, and a dry pad. In some embodiments, the substrate is selected from the group consisting of a tissue, a facial tissue, a bath tissue, a baby wipe, a personal care wipe, a makeup removal wipe, a personal protective wipe, a nursing pad, a cosmetic wipe, a perinea wipe, a disposable washcloth, a bath wipe, a cleaning wipe, a hydrogel moisturizing mask, a facial mask, a hand mask, an eye mask, and a disinfecting wipe.

In some embodiments, at least one face of the substrate is treated with the silk personal care composition or the silk fibroin fragment composition in an amount ranging from about 0.1 wt. % to about 25.0 wt. % by the total weight of the dried substrate. In some embodiments, at least one face of the substrate is treated with the silk personal care composition or the silk fibroin fragment composition in an amount ranging from about 0.5 wt. % to about 20.0% by the total weight of the dried substrate. The hydrogel substrate is impregnated with the silk personal care composition or the silk fibroin fragment composition in an amount ranging from about 0.1 wt. % to about 25 wt. % by the total weight of the hydrogel. The hydrogel substrate is impregnated with the silk personal care composition or the silk fibroin fragment composition in an amount ranging from about 0.5 wt. % to about 20.0 wt. %, by the total weight of the hydrogel.

In some embodiments, the substrate can be prepared according to conventional processes known to those skilled in the art. The substrate may be creped or uncreped.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

The compositions of this disclosure may be made by various methods known in the art. Such methods include those of the following examples, as well as the methods specifically exemplified below. Modifications of such methods that involve techniques commonly practiced in the art of personal care products may also be used.

### Materials and General Methods

### Materials

Pure silk fibroin-based protein (6.09% and 6.3%) was prepared according to the methods set forth in the Examples below. All surface activity, surface tension or elasticity was carried out at the same concentration of protein. Caprylic/capric glucoside (ORAMIX™ CG 110) was acquired from Seppic (Fairfield, N.J.). Other surfactants SLES (Sulfochem™ ES-1 Surfactant. 25.5%) and CAPB (Chembe-taine™ ACB Surfactant, 36%) were obtained from Lubrizol (Cleveland, Ohio). Rhamnolipid (Rheance One, 48.7%) and sophorolipid solution (Rewoferm SL One, 40%) were obtained from Evonik Industries (Essen, Germany). Xanthan Gum was purchased from Tokyo Chemical Industry Co., LTD (Tokyo, Japan) and k-carrageenan was purchased from Sigma Life Sciences (Milwaukee, Wis.).

### Mechanical Rheology

TA instrument DHR-3 rheometer (Manhattan College, Bronx, N.Y.) was used to measure flow curves and storage modulus (G′) and loss modulus (G″) frequency response. A 25 mm parallel plate was used for each experiment with a peltier plate controlling the temperature at 25° C. Before the frequency sweep to extrapolate G′ and G″, an amplitude sweep was run to determine the appropriate strain percent from the linear viscoelastic region of each sample.

### Du Noüy Ring Method

Surface tension at the air-water interface was measured at 20° C. with the Du Noüy ring technique on the Attension Sigma 701 Tensiometer (Manhattan College, Bronx, N.Y.). A small vessel was used for each test with 20 mL of sample and wait time of three hours was used before the du Noüy ring began to measure the surface tension.

### Test Sample Preparation

All samples were kept at a total concentration of 6 wt. %, with varied amounts of silk protein decreasing starting at 6.0 wt. % and surfactant amounts increasing. Deionized water was used as the solvent for each sample. Various ratios of silk protein and surfactants with deionized water used as the solvent were prepared in the glass vials. All samples were shaken lightly after being made and were left standing still for twenty-four hours in the fridge at 4° C. to become homogeneous. The solution of surfactant blend of silk protein and glucoside combination having preferred concentration ratio (5.5 wt. % silk+0.5 wt. % glucoside) was then mixed with various amounts of xanthan gum or carrageenan (thickener). The samples were prepared following the addition to the glass vials in the order of silk protein, glucoside, deionized water and thickener last. Thickener was added to the solution of silk protein, glucoside and deionized water slowly while the sample was stirred and heated at 45° C. until it was uniform. The samples were then left stay still for twenty-four hours in the fridge at 4° C. before being tested. All samples were made at a total of 20 mL and there was no dilution.

### Example 1. Preparation of Aqueous Silk Solution

Silk solutions of various molecular weights and/or combinations of molecular weights can be optimized for specific applications. The following provides an example of this process but it not intended to be limiting in application or formulation.

Methods of making silk fibroin or silk fibroin fragments and their applications in various fields are known and are described for example in U.S. Pat. Nos. 9,187,538, 9,511,012, 9,517,191, 9,522,107, 9,522,108, 9,545,369, and 10,166,177, 10,287,728 and 10,301,768, all of which are incorporated herein in their entireties.

The raw silk cocoons from the silkworm Bombyx mori was cut into pieces. The pieces silk cocoons were processed in an aqueous solution of Na₂CO₃ at about 100° C. for about 60 minutes to remove sericin (degumming). The volume of the water used equals about 0.4× raw silk weight and the amount of Na₂CO₃ is about 0.848× the weight of the raw silk cocoon pieces. The resulting degummed silk cocoon pieces were rinsed with deionized water three times at about 60° C. (20 minutes per rinse). The volume of rinse water for each cycle was 0.2 L×the weight of the raw silk cocoon pieces. The excess water from the degummed silk cocoon pieces was removed. After the DI water washing step, the wet degummed silk cocoon pieces were dried at room temperature. The degummed silk cocoon pieces were mixed with a LiBr solution, and the mixture was heated to about 100° C. The warmed mixture was placed in a dry oven and was heated at about 100° C. for about 60 minutes to achieve complete dissolution of the native silk protein. The resulting silk fibroin solution was filtered and dialyzed using Tangential Flow Filtration (TFF) and a 10 kDa membrane against deionized water for 72 hours. The resulting silk fibroin aqueous solution has a concentration of about 8.5 wt. %. Then, 8.5% silk solution was diluted with water to result in a 1.0% w/v silk solution. TFF can then be used to further concentrate the pure silk solution to a concentration of 20.0% w/w silk to water.

Each process step from raw cocoons to dialysis is scalable to increase efficiency in manufacturing. Whole cocoons are currently purchased as the raw material, but pre-cleaned cocoons or non-heat treated cocoons, where worm removal leaves minimal debris, have also been used. Cutting and cleaning the cocoons is a manual process, however for scalability this process could be made less labor intensive by, for example, using an automated machine in combination with compressed air to remove the worm and any particulates, or using a cutting mill to cut the cocoons into smaller pieces.

The degumming step, currently performed in small batches, could be completed in a larger vessel, for example an industrial washing machine where temperatures at or in between 60° C. to 100° C. can be maintained. The rinsing step could also be completed in the industrial washing machine, eliminating the manual rinse cycles.

Dissolution of the silk in LiBr solution could occur in a vessel other than a convection oven, for example a stirred tank reactor.

Dialyzing the silk through a series of water changes is a manual and time intensive process, which could be accelerated by changing certain parameters, for example diluting the silk solution prior to dialysis. The dialysis process could be scaled for manufacturing by using semi-automated equipment, for example a tangential flow filtration system.

Varying degumming conditions (i.e., time and temperature), LiBr solution parameters (i.e., concentration) and dissolution parameters (i.e., duration and temperature) results in aqueous silk solutions with different viscosities, homogeneities, and colors. Increasing the temperature for degumming process, lengthening the degumming time, using a higher temperature LiBr solution at emersion and over time when dissolving the silk and increasing the time at temperature (e.g., in an oven as shown here, or an alternative heat source) all resulted in less viscous and more homogeneous solvent and silk solutions. While almost all parameters resulted in a viable silk solution, methods that allow complete dissolution to be achieved in fewer than 4 to 6 hours are preferred for process scalability.

In some embodiments, the silk solutions are prepared under various preparation condition parameters such as: 90° C. 30 min, 90° C. 60 min, 100° C. 30 min, and 100° C. 60 min. Briefly, 9.3 M LiBr was prepared and allowed to sit at room temperature for at least 30 minutes. 5 mL of LiBr solution was added to 1.25 g of silk and placed in the 60° C. oven. Samples from each set were removed at 4, 6, 8, 12, 24, 168 and 192 hours. The remaining sample was photographed.

In some embodiments, the silk solutions are prepared under various preparation condition parameters such as: 90° C. 30 min, 90° C. 60 min, 100° C. 30 min, and 100° C. 60 min. Briefly, 9.3 M LiBr solution was heated to one of four temperatures: 60° C., 80° C., 100° C. or boiling. 5 mL of hot LiBr solution was added to 1.25 g of silk and placed in the 60° C. oven. Samples from each set were removed at 1, 4 and 6 hours. The remaining sample was photographed.

In some embodiments, the silk solutions are prepared under various preparation condition parameters such as: Four different silk extraction combinations were used: 90° C. 30 min, 90° C. 60 min, 100° C. 30 min, and 100° C. 60 min. Briefly, 9.3 M LiBr solution was heated to one of four temperatures: 60° C., 80° C., 100° C. or boiling. 5 mL of hot LiBr solution was added to 1.25 g of silk and placed in the oven at the same temperature of the LiBr. Samples from each set were removed at 1, 4 and 6 hours. 1 mL of each sample was added to 7.5 mL of 9.3 M LiBr and refrigerated for viscosity testing. The remaining sample was photographed.

Molecular weight of the silk protein fragments may be controlled based upon the specific parameters utilized during the extraction step, including extraction time and temperature; specific parameters utilized during the dissolution step, including the LiBr temperature at the time of submersion of the silk in to the lithium bromide and time that the solution is maintained at specific temperatures; and specific parameters utilized during the filtration step. By controlling process parameters using the disclosed methods, it is possible to create SPF mixture solutions with polydispersity equal to or lower than 2.5 at a variety of different molecular weight ranging from 5 kDa to 200 kDa, more preferably between 10 kDa and 80 kDa. By altering process parameters to achieve silk solutions with different molecular weights, a range of fragment mixture end products, with desired polydispersity of equal to or less than 2.5 may be targeted based upon the desired performance requirements. For example, a lower molecular weight silk film containing a drug may have a faster release rate compared to a higher molecular weight film making it ideal for a daily delivery vehicle in consumer cosmetics. Additionally, SPF mixture solutions with a polydispersity of greater than 2.5 can be achieved. Further, two solutions with different average molecular weights and polydispersities can be mixed to create combination solutions. Alternatively, a liquid silk gland (100% sericin free silk protein) that has been removed directly from a worm could be used in combination with any of the SPF mixture solutions of the present disclosure. Molecular weight of the pure silk fibroin-based protein fragment composition was determined using High Pressure Liquid Chromatography (HPLC) with a Refractive Index Detector (RID). Polydispersity was calculated using Cirrus GPC Online GPC/SEC Software Version 3.3 (Agilent).

Parameters were varied during the processing of raw silk cocoons into silk solution. Varying these parameters affected the MW of the resulting silk solution. Parameters manipulated included (i) time and temperature of extraction, (ii) temperature of LiBr, (iii) temperature of dissolution oven, and (iv) dissolution time. Experiments were carried out to determine the effect of varying the extraction time. Tables 1-6 summarize the results. Below is a summary:
A sericin extraction time of 30 minutes resulted in larger molecular weight than a sericin extraction time of 60 minutes
Molecular weight decreases with time in the oven
140° C. LiBr and oven resulted in the low end of the confidence interval to be below a molecular weight of 9500 Da
30 min extraction at the 1 hour and 4 hour time points have undigested silk
30 min extraction at the 1 hour time point resulted in a significantly high molecular weight with the low end of the confidence interval being 35,000 Da
The range of molecular weight reached for the high end of the confidence interval was 18000 to 216000 Da (important for offering solutions with specified upper limit).

TABLE 1
The effect of extraction time (30 min vs 60 min) on molecular weight of silk processed
under the conditions of 100° C. Extraction Temperature, 100° C. Lithium Bromide (LiBr)
and 100° C. Oven Dissolution (Oven/Dissolution Time was varied).

| Boil Time | Oven Time | Average Mw | Std dev | Confidence Interval | | PD |
|---|---|---|---|---|---|---|
| 30 | 1 | 57247 | 12780 | 35093 | 93387 | 1.63 |
| 60 | 1 | 31520 | 1387 | 11633 | 85407 | 2.71 |
| 30 | 4 | 40973 | 2632 | 14268 | 117658 | 2.87 |
| 60 | 4 | 25082 | 1248 | 10520 | 59803 | 2.38 |
| 30 | 6 | 25604 | 1405 | 10252 | 63943 | 2.50 |
| 60 | 6 | 20980 | 1262 | 10073 | 43695 | 2.08 |

TABLE 2
The effect of extraction time (30 min vs 60 min) on molecular weight of silk processed
under the conditions of 100° C. Extraction Temperature, boiling Lithium Bromide
(LiBr) and 60° C. Oven Dissolution for 4 hr.

| | Boil | Average | Std | Confidence | | |
|---|---|---|---|---|---|---|
| Sample | Time | Mw | dev | Interval | | PD |
| 30 min, 4 hr | 30 | 49656 | 4580 | 17306 | 142478 | 2.87 |
| 60 min, 4 hr | 60 | 30042 | 1536 | 11183 | 80705 | 2.69 |

TABLE 3
The effect of extraction time (30 min vs 60 min) on molecular weight of silk processed
under the conditions of 100° C. Extraction Temperature, 60° C. Lithium Bromide (LiBr)
and 60° C. Oven Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 30 min, 1 hr | 30 | 1 | 58436 | | 22201 | 153809 | 2.63 |
| 60 min, 1 hr | 60 | 1 | 31700 | | 11931 | 84224 | 2.66 |
| 30 min, 4 hr | 30 | 4 | 61956.5 | 13337 | 21463 | 178847 | 2.89 |
| 60 min, 4 hr | 60 | 4 | 25578.5 | 2446 | 9979 | 65564 | 2.56 |

TABLE 4
The effect of extraction time (30 min vs 60 min) on
molecular weight of silk processed under the conditions
of 100° C. Extraction Temperature, 80° C. Lithium
Bromide (LiBr) and 80° C. Oven Dissolution for 6 hr.

| | | Average | Std | Confidence | | |
|---|---|---|---|---|---|---|
| Sample | Boil Time | Mw | dev | Interval | | PD |
| 30 min, 6 hr | 30 | 63510 | | 18693 | 215775 | 3.40 |
| 60 min, 6 hr | 60 | 25164 | 238 | 9637 | 65706 | 2.61 |

TABLE 5
The effect of extraction time (30 min vs 60 min) on
molecular weight of silk processed under the conditions
of 100° C. Extraction Temperature, 80° C. Lithium Bromide (LiBr)
and 60° C. Oven Dissolution (Oven/Dissolution Time was varied).

| | Boil | Oven | Average | | Confidence | | |
|---|---|---|---|---|---|---|---|
| Sample | Time | Time | Mw | Std dev | Interval | | PD |
| 30 min, 4 hr | 30 | 4 | 59202 | 14028 | 19073 | 183760 | 3.10 |
| 60 min, 4 hr | 60 | 4 | 26312.5 | 637 | 10266 | 67442 | 2.56 |
| 30 min, 6 hr | 30 | 6 | 46824 | | 18076 | 121293 | 2.59 |
| 60 min, 6 hr | 60 | 6 | 26353 | | 10168 | 68302 | 2.59 |

TABLE 6
The effect of extraction time (30 min vs 60 min) on
molecular weight of silk processed under the conditions of
100° C. Extraction Temperature, 140° C. Lithium Bromide (LiBr)
and 140° C. Oven Dissolution (Oven/Dissolution Time was varied).

| | Boil | Oven | Average | | Confidence | | |
|---|---|---|---|---|---|---|---|
| Sample | Time | Time | Mw | Std dev | Interval | | PD |
| 30 min, 4 hr | 30 | 4 | 9024.5 | 1102 | 4493 | 18127 | 2.00865 |
| 60 min, 4 hr | 60 | 4 | 15548 | | 6954 | 34762 | 2.2358 |
| 30 min, 6 hr | 30 | 6 | 13021 | | 5987 | 28319 | 2.1749 |
| 60 min, 6 hr | 60 | 6 | 10888 | | 5364 | 22100 | 2.0298 |

Experiments were carried out to determine the effect of varying the extraction temperature. Table 7 summarizes the results. Below is a summary:
Sericin extraction at 90° C. resulted in higher MW than sericin extraction at 100° C. extraction
Both 90° C. and 100° C. show decreasing MW over time in the oven

TABLE 7
The effect of extraction temperature (90° C. vs. 100° C.) on
molecular weight of silk processed under the conditions of
60 min. Extraction Temperature, 100° C. Lithium Bromide
(LiBr) and 100° C. Oven Dissolution
(Oven/Dissolution Time was varied).

| | Boil | Oven | Average | | Confidence | | |
|---|---|---|---|---|---|---|---|
| Sample | Time | Time | Mw | Std dev | Interval | | PD |
| 90° C., 4 hr | 60 | 4 | 37308 | 4204 | 13368 | 104119 | 2.79 |
| 100° C., 4 hr | 60 | 4 | 25082 | 1248 | 10520 | 59804 | 2.38 |
| 90° C., 6 hr | 60 | 6 | 34224 | 1135 | 12717 | 92100 | 2.69 |
| 100° C., 6 hr | 60 | 6 | 20980 | 1262 | 10073 | 43694 | 2.08 |

Experiments were carried out to determine the effect of varying the Lithium Bromide (LiBr) temperature when added to silk. Tables 8-9 summarize the results. Below is a summary:
No impact on molecular weight or confidence interval (all CI˜10500-6500 Da)
Studies illustrated that the temperature of LiBr-silk dissolution, as LiBr is added and begins dissolving, rapidly drops below the original LiBr temperature due to the majority of the mass being silk at room temperature

TABLE 8
The effect of Lithium Bromide (LiBr) temperature
on molecular weight of silk processed under the conditions
of 60 min. Extraction Time, 100° C. Extraction
Temperature and 60° C. Oven Dissolution
(Oven/Dissolution Time was varied).

| | LiBr | | | | | | |
|---|---|---|---|---|---|---|---|
| | Temp | Oven | Average | Std | Confidence | | |
| Sample | (° C.) | Time | Mw | dev | Interval | | PD |
| 60° C. LiBr, | 60 | 1 | 31700 | | 11931 | 84223 | 2.66 |
| 1 hr | | | | | | | |
| 100° C. LiBr, | 100 | 1 | 27907 | 200 | 10735 | 72552 | 2.60 |
| 1 hr | | | | | | | |
| RT LiBr, 4 hr | RT | 4 | 29217 | 1082 | 10789 | 79119 | 2.71 |
| 60° C. LiBr, | 60 | 4 | 25578 | 2445 | 9978 | 65564 | 2.56 |
| 4 hr | | | | | | | |
| 80° C. LiBr, | 80 | 4 | 26312 | 637 | 10265 | 67441 | 2.56 |
| 4 hr | | | | | | | |
| 100° C. LiBr, | 100 | 4 | 27681 | 1729 | 11279 | 67931 | 2.45 |
| 4 hr | | | | | | | |
| Boil LiBr, 4 hr | Boil | 4 | 30042 | 1535 | 11183 | 80704 | 2.69 |
| RT LiBr, 6 hr | RT | 6 | 26543 | 1893 | 10783 | 65332 | 2.46 |
| 80° C. LiBr, | 80 | 6 | 26353 | | 10167 | 68301 | 2.59 |
| 6 hr | | | | | | | |
| 100° C. LiBr, | 100 | 6 | 27150 | 916 | 11020 | 66889 | 2.46 |
| 6 hr | | | | | | | |

TABLE 9
The effect of Lithium Bromide (LiBr) temperature on
molecular weight of silk processed under the conditions
of 30 min. Extraction Time, 100° C. Extraction
Temperature and 60° C. Oven Dissolution
(Oven/Dissolution Time was varied).

| | LiBr | | | | | | |
|---|---|---|---|---|---|---|---|
| | Temp | Oven | Average | Std | Confidence | | |
| Sample | (° C.) | Time | Mw | dev | Interval | | PD |
| 60° C. LiBr, | 60 | 4 | 61956 | 13336 | 21463 | 178847 | 2.89 |
| 4 hr | | | | | | | |
| 80° C. LiBr, | 80 | 4 | 59202 | 14027 | 19073 | 183760 | 3.10 |
| 4 hr | | | | | | | |
| 100° C. LiBr, | 100 | 4 | 47853 | | 19757 | 115899 | 2.42 |
| 4 hr | | | | | | | |
| 80° C. LiBr, | 80 | 6 | 46824 | | 18075 | 121292 | 2.59 |
| 6 hr | | | | | | | |
| 100° C. LiBr, | 100 | 6 | 55421 | 8991 | 19152 | 160366 | 2.89 |
| 6 hr | | | | | | | |

Experiments were carried out to determine the effect of v oven/dissolution temperature. Tables 10-14 summarize the results. Below is a summary:
Oven temperature has less of an effect on 60 min extracted silk than 30 min extracted silk. Without wishing to be bound by theory, it is believed that the 30 min silk is less degraded during extraction and therefore the oven temperature has more of an effect on the larger MW, less degraded portion of the silk.
For 60° C. vs. 140° C. oven the 30 min extracted silk showed a very significant effect of lower MW at higher oven temp, while 60 min extracted silk had an effect but much less
The 140° C. oven resulted in a low end in the confidence interval at ˜6000 Da.

TABLE 10
The effect of oven/dissolution temperature
on molecular weight of silk processed under the conditions of
100° C. Extraction Temperature, 30 min. Extraction
Time, and 100° C. Lithium Bromide (LiBr)
(Oven/Dissolution Time was varied).

| Boil | Oven Temp | Oven | Average | | Confidence | | |
|---|---|---|---|---|---|---|---|
| Time | (° C.) | Time | Mw | Std dev | Interval | | PD |
| 30 | 60 | 4 | 47853 | | 19758 | 115900 | 2.42 |
| 30 | 100 | 4 | 40973 | 2632 | 14268 | 117658 | 2.87 |
| 30 | 60 | 6 | 55421 | 8992 | 19153 | 160366 | 2.89 |
| 30 | 100 | 6 | 25604 | 1405 | 10252 | 63943 | 2.50 |

TABLE 11
The effect of oven/dissolution temperature on
molecular weight of silk processed under the conditions
of 100° C. Extraction Temperature, 60 min. Extraction
Time, and 100° C. Lithium Bromide (LiBr)
(Oven/Dissolution Time was varied).

| Boil Time | Oven Temp | Oven | Average | | Confidence | | |
|---|---|---|---|---|---|---|---|
| (minutes) | (° C.) | Time | Mw | Std dev | Interval | | PD |
| 60 | 60 | 1 | 27908 | 200 | 10735 | 72552 | 2.60 |
| 60 | 100 | 1 | 31520 | 1387 | 11633 | 85407 | 2.71 |
| 60 | 60 | 4 | 27681 | 1730 | 11279 | 72552 | 2.62 |
| 60 | 100 | 4 | 25082 | 1248 | 10520 | 59803 | 2.38 |
| 60 | 60 | 6 | 27150 | 916 | 11020 | 66889 | 2.46 |
| 60 | 100 | 6 | 20980 | 1262 | 10073 | 43695 | 2.08 |

TABLE 12
The effect of oven/dissolution temperature on
molecular weight of silk processed under the conditions
of 100° C. Extraction Temperature, 60 min. Extraction
Time, and 140° C. Lithium Bromide (LiBr)
(Oven/Dissolution Time was varied).

| Boil | Oven | | | | | | |
|---|---|---|---|---|---|---|---|
| Time | Temp | Oven | Average | | Confidence | | |
| (minutes) | (° C.) | Time | Mw | Std dev | Interval | | PD |
| 60 | 60 | 4 | 30042 | 1536 | 11183 | 80705 | 2.69 |
| 60 | 140 | 4 | 15548 | | 7255 | 33322 | 2.14 |

TABLE 13
The effect of oven/dissolution temperature on
molecular weight of silk processed under the conditions
of 100° C. Extraction Temperature, 30 min. Extraction
Time, and 140° C. Lithium Bromide (LiBr)
(Oven/Dissolution Time was varied).

| | Oven | | | | | | |
|---|---|---|---|---|---|---|---|
| Boil Time | Temp | Oven | Average | | Confidence | | |
| (minutes) | (° C.) | Time | Mw | Std dev | Interval | | PD |
| 30 | 60 | 4 | 49656 | 4580 | 17306 | 142478 | 2.87 |
| 30 | 140 | 4 | 9025 | 1102 | 4493 | 18127 | 2.01 |
| 30 | 60 | 6 | 59383 | 11640 | 17641 | 199889 | 3.37 |
| 30 | 140 | 6 | 13021 | | 5987 | 28319 | 2.17 |

TABLE 14
The effect of oven/dissolution temperature on
molecular weight of silk processed under the conditions
of 100° C. Extraction Temperature, 60 min. Extraction
Time, and 80° C. Lithium Bromide (LiBr)
(Oven/Dissolution Time was varied).

| | Oven | | | | | | |
|---|---|---|---|---|---|---|---|
| Boil Time | Temp | Oven | Average | | Confidence | | |
| (minutes) | (° C.) | Time | Mw | Std dev | Interval | | PD |
| 60 | 60 | 4 | 26313 | 637 | 10266 | 67442 | 2.56 |
| 60 | 80 | 4 | 30308 | 4293 | 12279 | 74806 | 2.47 |
| 60 | 60 | 6 | 26353 | | 10168 | 68302 | 2.59 |
| 60 | 80 | 6 | 25164 | 238 | 9637 | 65706 | 2.61 |

The raw silk cocoons from the silkworm Bombyx mori was cut into pieces. The pieces of raw silk cocoons were boiled in an aqueous solution of Na₂CO₃ (about 100° C.) for a period of time between about 30 minutes to about 60 minutes to remove sericin (degumming). The volume of the water used equals about 0.4× raw silk weight and the amount of Na₂CO₃ is about 0.848× the weight of the raw silk cocoon pieces. The resulting degummed silk cocoon pieces were rinsed with deionized water three times at about 60° C. (20 minutes per rinse). The volume of rinse water for each cycle was 0.2 L×the weight of the raw silk cocoon pieces. The excess water from the degummed silk cocoon pieces was removed. After the DI water washing step, the wet degummed silk cocoon pieces were dried at room temperature. The degummed silk cocoon pieces were mixed with a LiBr solution, and the mixture was heated to about 100° C. The warmed mixture was placed in a dry oven and was heated at a temperature ranging from about 60° C. to about 140° C. for about 60 minutes to achieve complete dissolution of the native silk protein. The resulting solution was allowed to cool to room temperature and then was dialyzed to remove LiBr salts using a 3,500 Da MWCO membrane. Multiple exchanges were performed in Di water until Br⁻ ions were less than 1 ppm as determined in the hydrolyzed fibroin solution read on an Oakton Bromide (Br) double junction ion-selective electrode.

The resulting silk fibroin aqueous solution has a concentration of about 8.0% w/v containing pure silk fibroin-based protein fragments having an average weight average molecular weight selected from between about 6 kDa to about 16 kDa, about 17 kDa to about 39 kDa, and about 39 kDa to about 80 kDa and a polydispersity of between about 1.5 and about 3.0. The 8.0% w/v was diluted with DI water to provide a 1.0% w/v, 2.0% w/v, 3.0% w/v, 4.0% w/v, 5.0% w/v by the coating solution.

A variety of % silk concentrations have been produced through the use of Tangential Flow Filtration (TFF). In all cases a 1% silk solution was used as the input feed. A range of 750-18,000 mL of 1% silk solution was used as the starting volume. Solution is diafiltered in the TFF to remove lithium bromide. Once below a specified level of residual LiBr, solution undergoes ultrafiltration to increase the concentration through removal of water. See examples below.

Six (6) silk solutions were utilized in standard silk structures in accordance with standard methods in the literature with the following results:

Solution #1 is a silk concentration of 5.9 wt. %, average MW of 19.8 kDa and 2.2 PDI (made with a 60 min boil extraction, 100° C. LiBr dissolution for 1 hour).

Solution #2 is a silk concentration of 6.4 wt. % (made with a 30 min boil extraction, 60° C. LiBr dissolution for 4 hrs).

Solution #3 is a silk concentration of 6.17 wt. % (made with a 30 min boil extraction 100° C. LiBr dissolution for 1 hour).

Solution #4 is a silk concentration of 7.30 wt. %: A 7.30% silk solution was produced beginning with 30 minute extraction batches of 100 g silk cocoons per batch. Extracted silk fibers were then dissolved using 100° C. 9.3 M LiBr in a 100° C. oven for 1 hour. 100 g of silk fibers were dissolved per batch to create 20% silk in LiBr. Dissolved silk in LiBr was then diluted to 1% silk and filtered through a 5 μm filter to remove large debris. 15,500 mL of 1%, filtered silk solution was used as the starting volume/diafiltration volume for TFF. Once LiBr was removed, the solution was ultrafiltered to a volume around 1300 mL. 1262 mL of 7.30% silk was then collected. Water was added to the feed to help remove the remaining solution and 547 mL of 3.91% silk was then collected.

Solution #5 is a silk concentration of 6.44 wt. %: A 6.44 wt. % silk solution was produced beginning with 60 minute extraction batches of a mix of 25, 33, 50, 75 and 100 g silk cocoons per batch. Extracted silk fibers were then dissolved using 100° C. 9.3 M LiBr in a 100° C. oven for 1 hour. 35, 42, 50 and 71 g per batch of silk fibers were dissolved to create 20% silk in LiBr and combined. Dissolved silk in LiBr was then diluted to 1% silk and filtered through a 5 μm filter to remove large debris. 17,000 mL of 1%, filtered silk solution was used as the starting volume/diafiltration volume for TFF. Once LiBr was removed, the solution was ultrafiltered to a volume around 3000 mL. 1490 mL of 6.44% silk was then collected. Water was added to the feed to help remove the remaining solution and 1454 mL of 4.88% silk was then collected.

Solution #6 is a silk concentration of 2.70 wt. %: A 2.70% silk solution was produced beginning with 60-minute extraction batches of 25 g silk cocoons per batch. Extracted silk fibers were then dissolved using 100° C. 9.3 M LiBr in a 100° C. oven for 1 hour. 35.48 g of silk fibers were dissolved per batch to create 20% silk in LiBr. Dissolved silk in LiBr was then diluted to 1% silk and filtered through a 5 μm filter to remove large debris. 1000 mL of 1%, filtered silk solution was used as the starting volume/diafiltration volume for TFF. Once LiBr was removed, the solution was ultrafiltered to a volume around 300 mL. 312 mL of 2.7% silk was then collected.

The preparation of silk fibroin solutions with higher molecular weights is given in Table 15.

TABLE 15
Preparation and properties of silk fibroin solutions.

| | | | | | Average | |
|---|---|---|---|---|---|---|
| | | | | | weight | |
| | | | | | average | |
| | Extraction | Extraction | LiBr | | molecular | |
| Sample | Time | Temp | Temp | Oven/Sol'n | weight | Average |
| Name | (mins) | (° C.) | (° C.) | Temp | (kDa) | polydispersity |
| Group A | 60 | 100 | 100 | 100° C. | 34.7 | 2.94 |
| TFF | | | | oven | | |
| Group A | 60 | 100 | 100 | 100° C. | 44.7 | 3.17 |
| DIS | | | | oven | | |
| Group B | 60 | 100 | 100 | 100° C. | 41.6 | 3.07 |
| TFF | | | | sol'n | | |
| Group B DIS | 60 | 100 | 100 | 100° C. | 44.0 | 3.12 |
| | | | | sol'n | | |
| Group C | 60 | 100 | 140 | 140° C. | 10.9 | 3.19 |
| TFF | | | | sol'n | | |
| Group C DIS | 60 | 100 | 140 | 140° C. | | |
| | | | | sol'n | | |
| Group D | 30 | 90 | 60 | 60° C. sol'n | 129.7 | 2.56 |
| DIS | | | | | | |
| Group D FIL | 30 | 90 | 60 | 60° C. sol'n | 144.2 | 2.73 |
| Group E DIS | 15 | 100 | RT | 60° C. sol'n | 108.8 | 2.78 |
| Group E FIL | 15 | 100 | RT | 60° C. sol'n | 94.8 | 2.62 |

### Example 2. Emulsifying Capacity of Silk Protein Emulsifiers

The calculated HLB value for silk fibroin protein fragment is 6.2. The HLB value for sorbitan laurate is 8.6. Both the silk fibroin protein fragment and sorbitan laurate are in the same class of emulsifier for water-in-oil emulsions.

The emulsification efficiency of the silk fibroin protein fragment emulsifier was determined by adding 1% silk fibroin protein fragment solution into a 45 mL conical polypropylene centrifuge tube. Jojoba oil or squalene was added into the silk aqueous solution. The mixture was then homogenized using a homogenizer at a speed of 10,000 rpm for 2 minutes to produce water-in-oil emulsion. Creaming stability of the emulsion was determined.

The emulsification efficiency of the medium molecular weight silk fibroin protein fragments and sorbitan laurate (Span 20) on oils (e.g., jojoba oil) was measured based on the concentration of 1% (w/w) emulsifier. By adding various volumes of squalene or jojoba oil to that SPF water solution at room temperature (the volume ratio of SPF solution to the oil phase) (W/O) was altered from 1:4, 2:3, 3:2 to 4:1), after homogenization at 10,000 rpm for 2 min (emulsification), emulsions formed (FIGS. 1A-B). At a higher volume of SPF solution (W/O, 4:1), the milky emulsions appeared in the oil phases and the aqueous phases remained transparent, suggesting the formation of water-in-oil emulsions. The milky emulsions in the aqueous phases were expanded following an increased 0/W ratio, due to the lower density of emulsions dominated by the oil core and their smaller size.

Samples Sp. 3, Sp.4, Sp. 5 and Sp. 6 of FIG. 1A illustrated silk fibroin protein fragment emulsified jojoba oil at increasing volume ratio of jojoba oil to water from 0.2, 0.4, 0.6, 0.8 in a water/oil system. Samples Sp. 7, Sp. 8, Sp. 9 and Sp. 10 of FIG. 1B illustrated sorbitan laurate emulsified jojoba oil at increasing volume ratio of jojoba oil to water from 0.2, 0.4, 0.6, 0.8 in a water/oil system.

The aqueous layers below the emulsion layers were opaque rather than transparent, without wishing to be bound by any particular theory, may indicate the existence of smaller oil-filled microcapsules (serum). No serum was observed for an oil fraction of 0.8 with either silk fibroin or sorbitan laurate. There was no significant observed difference in the emulsification efficiency of the silk fibroin protein fragments and sorbitan laurate (FIGS. 1A-1B).

### Example 3. Creaming Stability

The process by which an emulsion completely breaks (coalescence), i.e., the system separates into bulk oil and water phases, is generally considered to be governed by four different droplet loss mechanisms, i.e., Brownian flocculation, creaming, Ostwald ripening and disproportionation.

Creaming is the principal process by which the disperse phase separates from an emulsion and is typically the precursor to coalescence. Creaming is not an actual breaking but a separation of the emulsion into two emulsions, one of which (the cream) is richer in the disperse phase than the other is.

The processes of creaming, flocculation and coalescence are well demonstrated by taking an emulsion of limited stability and centrifuging it at low speeds or various lengths of time. Initially, for oils with a density less than water, a “rising” of the cream is observed. Then, as larger droplets rise and concentrate, they begin to appear at the top. Finally, the drops coalesce to form a separate layer of oil on top.

As used herein, the “creaming index” refers to a method for direct measuring of creaming stability of the emulsion by visual observation. Creaming index (CI) is defined as percent of fraction of the serum volume (HL) by the total emulsion volume (HE) (See Eq. 1 below). The smaller CI numeric value indicates more stable emulsion.

Creaming index=Serum Volume/Total Emulsion Volume×100 Eq. 1

For creaming stability study and creaming index determination, various jojoba oil emulsions using 1% (w/w) silk fibroin protein fragment and sorbitan laurate emulsifier were prepared according the process described above. The testing results of creaming index for various jojoba oil emulsions stabilized by silk fibroin and sorbitan laurate were summarized in FIG. 2 and Table 15 below.

TABLE 15
Jojoba oil emulsions and their Creaming Index

| Entry | | Sp. 3 | Sp. 4 | Sp. 5 | Sp. 6 | Sp. 7 | Sp. 8 | Sp. 9 | Sp. 10 |
|---|---|---|---|---|---|---|---|---|---|
| Aqueous | Water | 79 | 59 | 39 | 19 | 79 | 50 | 39 | 19 |
| phase | (% v/v) | | | | | | | | |
| Oil phase | Jojoba oil | 20 | 40 | 60 | 80 | 20 | 40 | 60 | 80 |
| | (% v/v) | | | | | | | | |
| emulsifier | Mid MW | 1 | 1 | 1 | 1 | | | | |
| | silk | | | | | | | | |
| | (% w/v) | | | | | | | | |
| | Sorbitan | | | | | 1 | 1 | 1 | 1 |
| | laurate | | | | | | | | |
| | (% w/v) | | | | | | | | |
| Emulsion | Creaming | 58% | 38% | 13% | 0 | 70% | 45% | 20% | 0 |
| stability | index^{a} | | | | | | | | |

^{a}Creaming index (CI) = ((serum volume)/(total emulsion volume)) × 100%. Smaller CI indicates more stable emulsion. For jojoba/water (80/20) system, the amount of mid-MW silk 0.6% w/v to 2.4% w/v

### Example 4. Accelerated Emulsion Stability Studies

Silk fibroin fragments having different molecular weight ranges were used to emulsify oils having both high polarity index (squalane) and low polarity index (jojoba oil).

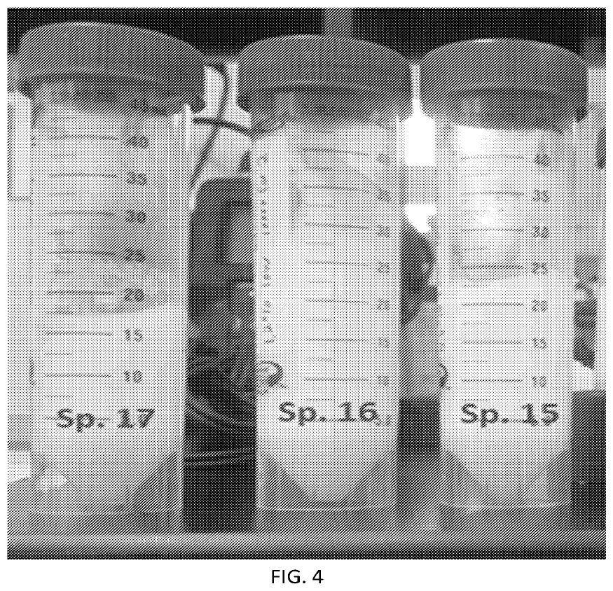

For accelerated emulsion stability determination, various jojoba oil and squalane emulsions by adding 20% v/v of a silk solution containing 0.6%, 1.2% and 2.4% (w/v) silk fibroin protein fragment emulsifier and 80% v/v of an oil phase to a 45 mL polypropylene centrifuge tube. The mixture was then homogenized using a homogenizer at a speed of 10,000 rpm for 2 minutes to produce water-in-oil emulsion (See FIGS. 3-4).

The above formed emulsions were transferred in to a 1.5 mL Eppendorf tube for the accelerated emulsion stability testing. The 1.5 mL Eppendorf tube was filled with the emulsion and then centrifuged. If at the end of the centrifugation there was no serum the centrifugation speed was increased and the experiment continued until serum separation was observed. The accelerated emulsion stability was determined by measuring oil separation expressed in unit “mm” after subjecting the emulsions to centrifugation at increasing speeds of 500 rpm, 900 rpm, 1000 rpm and 150 rpm. The smaller numeric value of oil separation indicates more stable emulsion.

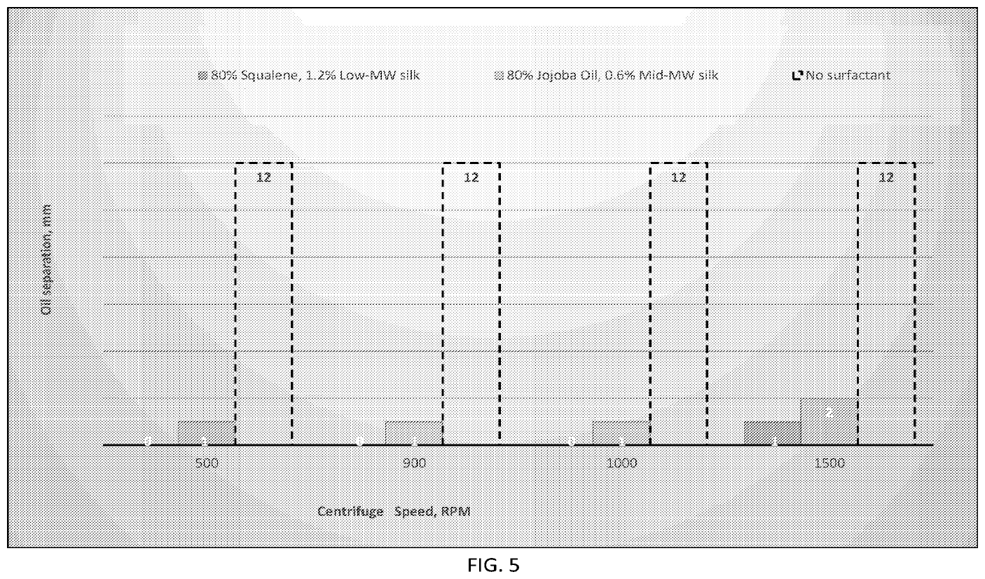

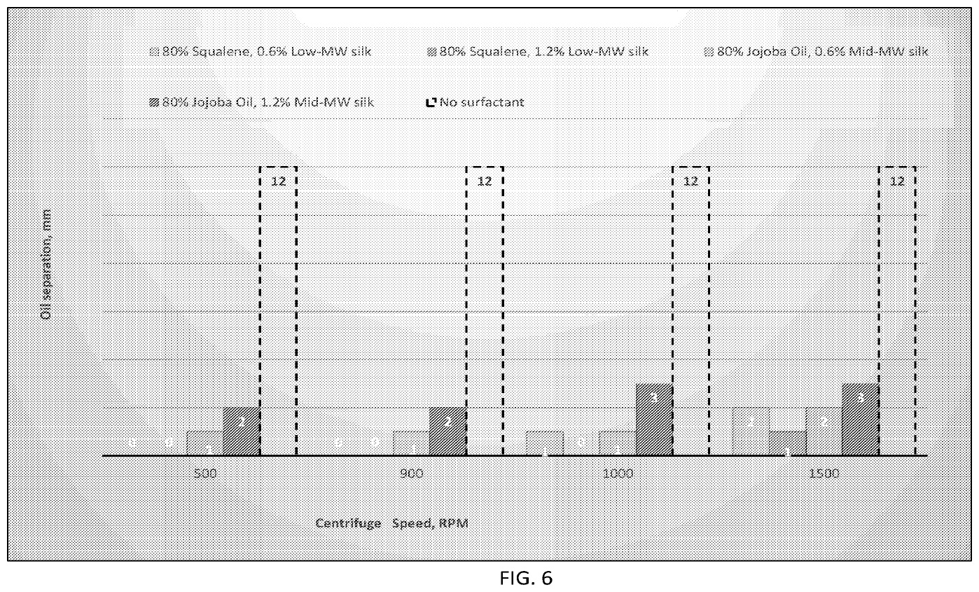

The testing results of oil separation for various jojoba oil and squalene emulsions stabilized by silk fibroin were summarized in FIGS. 5-6 and Table 16 below.

TABLE 16
Silk fibroin protein fragment emulsified water-in-oil emulsion

| Entry | | Sp. 12 | Sp. 13 | Sp. 6 | Sp. 14 | Sp. 15 | Sp. 10 | Sp. 16 | Sp.17 |
|---|---|---|---|---|---|---|---|---|---|
| Aqueous | Water | 19.2 | 19.0 | 18.8 | 17.6 | 19.4 | 19.0 | 18.8 | 17.6 |
| phase | (% v/v) | | | | | | | | |
| Oil phase | Jojoba oil | 80 | 80 | 80 | 80 | | | | |
| | (% v/v) | | | | | | | | |
| | Squalane | | | | | 80 | 80 | 80 | 80 |
| | (% v/v) | | | | | | | | |
| emulsifier | Low MW | | | | | 0.6 | 1.0 | 1.2 | 2.4 |
| | silk | | | | | | | | |
| | (% w/v) | | | | | | | | |
| | Mid MW | 0.6 | 1.0 | 1.2 | 2.4 | | | | |
| | silk | | | | | | | | |
| | (% w/v) | | | | | | | | |
| | Oil | 2 | | 3 | | 1 | | 2 | |
| | separation | | | | | | | | |
| | (mm) | | | | | | | | |

It was found that low molecular weight silk fibroin protein fragment is a better emulsifier for oil having high polarity index (i.e., squalene), and medium molecular weight silk fibroin protein fragment is a better emulsifier for oil having low polarity index (i.e., jojoba oil) (See FIG. 5). For squalane emulsions stabilized by the low molecular weight silk fibroin, emulsion stability increases with the increase of silk emulsifier concentration. For jojoba oil emulsions stabilized by the medium molecular weight silk fibroin, emulsion stability decreases with the increase of silk emulsifier concentration (See FIG. 6).

### Example 5. Silk Fibroin/Surfactant Blend Stabilized Foam

### Example 5A. Foaming Test

This example characterize the adoption of silk fibroin at the air-water interface. Surface tension measurement and some foaming studies were used to evaluate the interactions if silk fibroin protein with other conventional surfactant such as caprylyl/capryl glucoside. Foaming test was performed by incorporating air in a surfactant/water mixture, containing silk fibroin protein or natural surfactants for stabilization.

Various aqueous surfactant foams were prepared by adding 1 mL of 6% w/v of various surfactants solution in water for caprylyl/capryl glucoside (Sp. 16), rhamnolipid (Sp. 17), a blend of 1% w/v silk protein and 5% w/v caprylyl/capryl glucoside (Sp. 18), and sophorolipid (Sp. 19) to a 2 mL glass vial. Each sample vial was shaken uniformly for 10 seconds to produce foam (air bubbles stabilized by surfactant film). The vials containing the foams were allow to stand for 15 seconds before the foam volume was recorded as t=0 minute, this determined the foamability. The foam stability was then measured by monitoring the foam volume over a period of 45 minutes. The foam volume was recorded at time interval of 5, 15, 30 and 45 minutes (See FIGS. 7A-E).

As can be seen in FIGS. 7A-7B, the foam stability of 6% w/v sophorolipid solution is not very good, the maximum foam volume produced at t=0 minutes is about 1 mL (See FIG. 7A). The foam volume of 6% w/v sophorolipid solution decreased significantly within 5 minutes (See FIG. 7B) and disappeared completely by 30 minutes (See FIG. 7D).

The 5% w/v silk fibroin and 1% w/v caprylyl/capryl glucoside blend solution produced the highest amount foam volume as compared with 6% w/v caprylyl/capryl glucoside, 6% w/v rhamnolipid, and 6% w/v sophorolipid (See FIG. 7A).

### Example 5B. Surface Tension Measurement

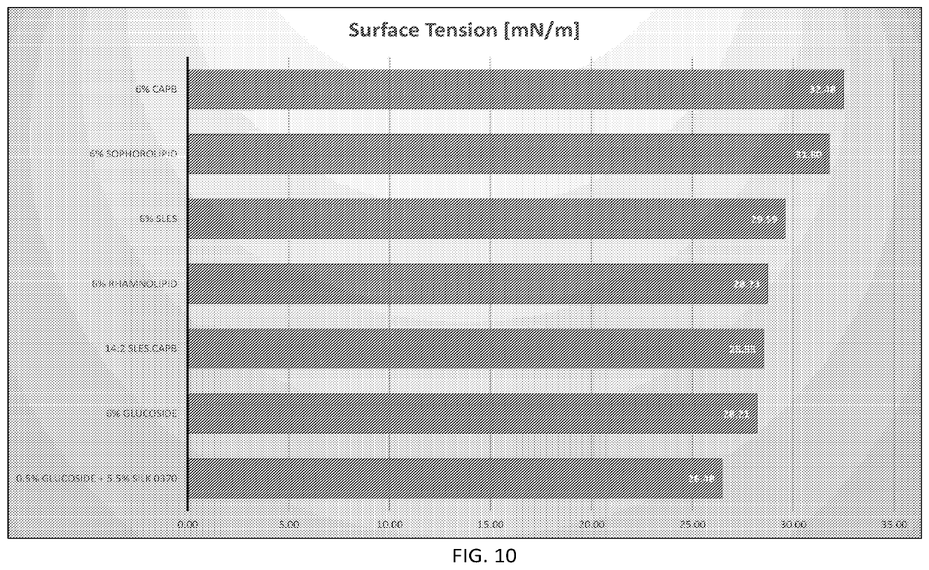

Surface tension measurements were conducted on silk fibroin solution at 6.0% w/v. The adoption of silk fibroin at the air-water interface caused a significant reduction in the surface tension. The surface tension of pure silk protein in aqueous solution is 48.127 mN/m, a reduction from the surface tension of pure water at 72 mN/m (See FIG. 8). Comparisons of surface tension reduction by various surfactant systems in 6% w/v surfactant aqueous solution is illustrated in FIG. 10.

Co-adsorption of silk fibroin with caprylyl/capryl glucoside can result in synergistic effects in term of surface activity that leads to more efficient use of the surfactant (e.g., less quantity will be needed to achieve the same surface tension).

It was found that the addition of a 1% w/v caprylyl/capryl glucoside emulsifier to 5% w/v silk fibroin solution, leads to synergistically decrease of the surface tensions at neutral pH from 48.127 mN/m of pure silk fibroin to 27.2 mN/m, even at very small amounts such as 0.3% v/w of caprylyl/capryl glucoside (26.5 mN/m) and 0.5% w/w caprylyl/capryl glucoside (26.5 mN/m) (See FIG. 8, FIG. 10) as compared with that of pure silk protein (48.127 mN/m) and 6% w/v pure caprylyl/capryl glucoside (29 mN/m). This suggested that the lowest surface tension resulted from the silk-glucoside blend might be the result of formation of surface active silk fibroin-glucoside complexes having higher surface activity as compared with either of the pure surfactants.

In contrast to the decreasing surface tension by the pure surfactant (See, FIG. 10), it was surprise to discover, for the silk-glucoside blend surfactant system here, that increasing the glucoside concentration in relative to silk fibroin protein fragments resulted in slight increase of surface tension from 26.2 mN/m at 0.5% w/v glucoside to 28.2 mN/m at 6% w/v glucoside (See FIG. 8). The surface tension increasement reached a plateau when the glucoside concentration reached 6% w/v and above. For example, adding 5% w/v caprylyl/capryl glucoside to the 5% w/v silk fibroin solution, the surface tension was increased from 26.4 mN/m to 28.2 mN/m. Lower concentration of glucoside (i.e. 0.3% w/v to 1.0 w/v) is preferred to form blend with silk fibroin fragments (See FIG. 8).

Lowering pH of the silk/glucoside 5%:1% blend aqueous solution from neutral pH 7.2 to 5.5 did not result in drastic reduction of the surface tension further (See FIG. 9). This result indicated that the adoption of silk fibroin at the air-water interface depending more on hydrophobic interactions rather than electrostatic interactions over the pH value studied.

This is a very attractive result in term of silk fibroin's potential use as a surfactant.

The pure silk fibroin peptide's ability to lower surface tension (48.127 mN/m for 6% w/b pure silk solution, FIG. 8) is not as good as the traditional surfactant, e.g., 6% CAPB (about 32.48 mN/m), 6% SLES (29.59 mN/m), 6% w/v sophorolipid (31.80 mN/m), 6% rhamnolipid (28.73 mN/m) (FIG. 10). The blend containing 0.5% w/v caprylyl/capryl glucoside and 5.5% silk fibroin protein fragment gave synergistic effects on reducing the surface tension (26.48 mN/m) (FIG. 10).

### Example 6. Thickening Agent/Silk Fibroin/Surfactant Blend Stabilized Foam

### Example 6A. Foaming Test

This example evaluated the foam stabilizing effects imparted by thickening agent carrageenan and xanthan gum. Foaming test was performed by incorporating air in a surfactant/water mixture, containing silk fibroin protein and thickening agent for stabilization.

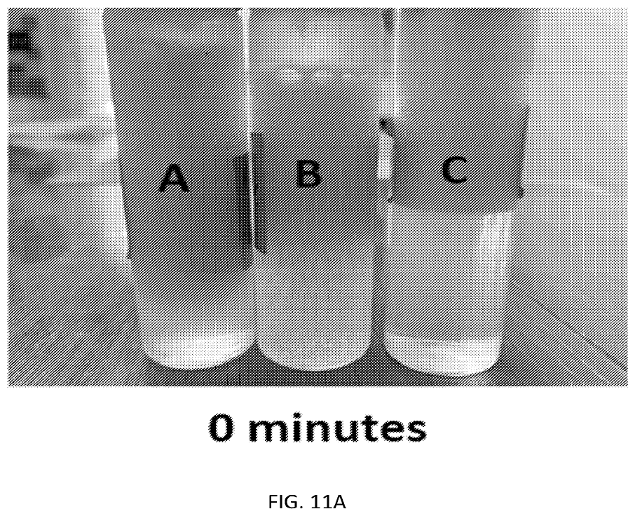

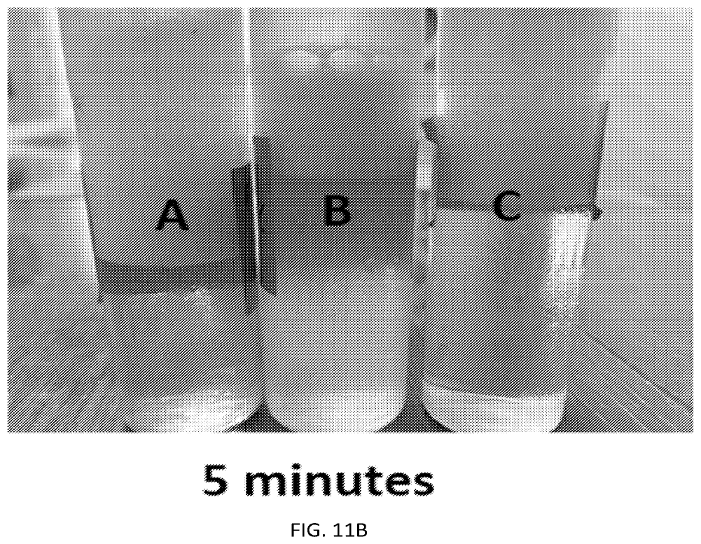

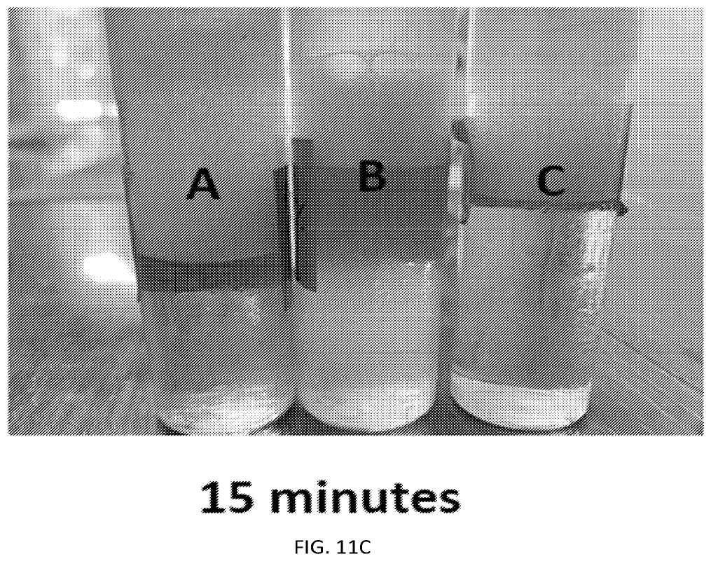

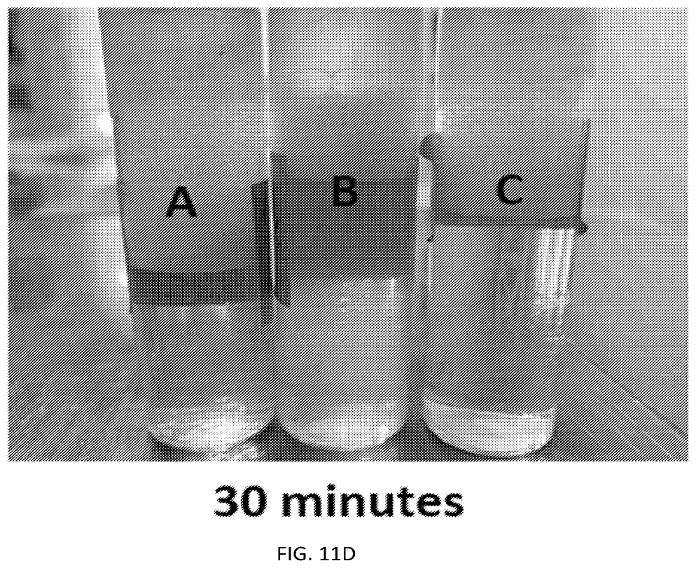

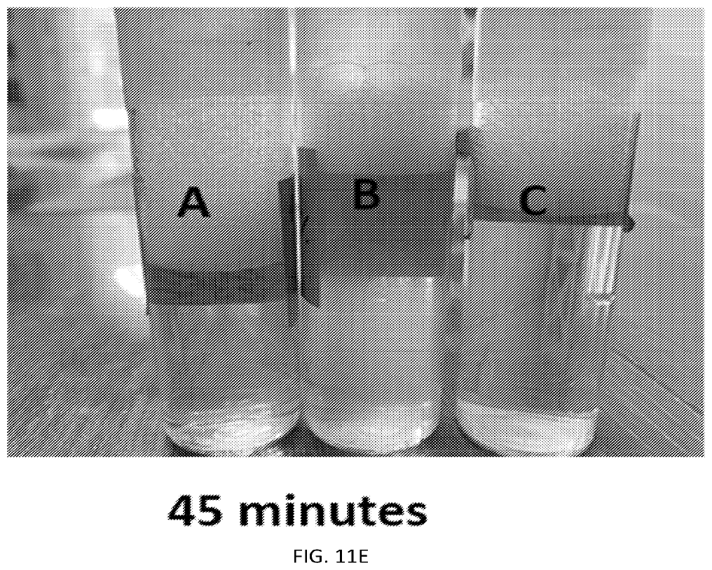

Various aqueous thickener stabilized surfactant foams and controlled foam without thickening agent were prepared by adding 0 g of thickener, 0.025 g of carrageenan (0.125% w/v), 0.025 g of xanthan gum (0.125% w/v) to 20 mL aqueous solution containing a blend of 5.5% w/v silk protein and 0.5% w/v glucoside to a glass vial. Each sample vial was shaken uniformly for 10 seconds to produce foam (air bubbles stabilized by surfactant film). The vials containing the foams were allow to stand for 15 seconds before the foam volume was recorded as t=0 minute, this determined the foamability. The foam stability was then measured by monitoring the foam volume over a period of 45 minutes. The foam volume was recorded at time interval of 5, 15, 30 and 45 minutes (See FIGS. 11A-E).

Noticeable densense and small bubbles are formed for all three test samples. As time increases, the stability of the bubbles decreases slightly with all samples.

### Example 6B. Surface Tension Measurement

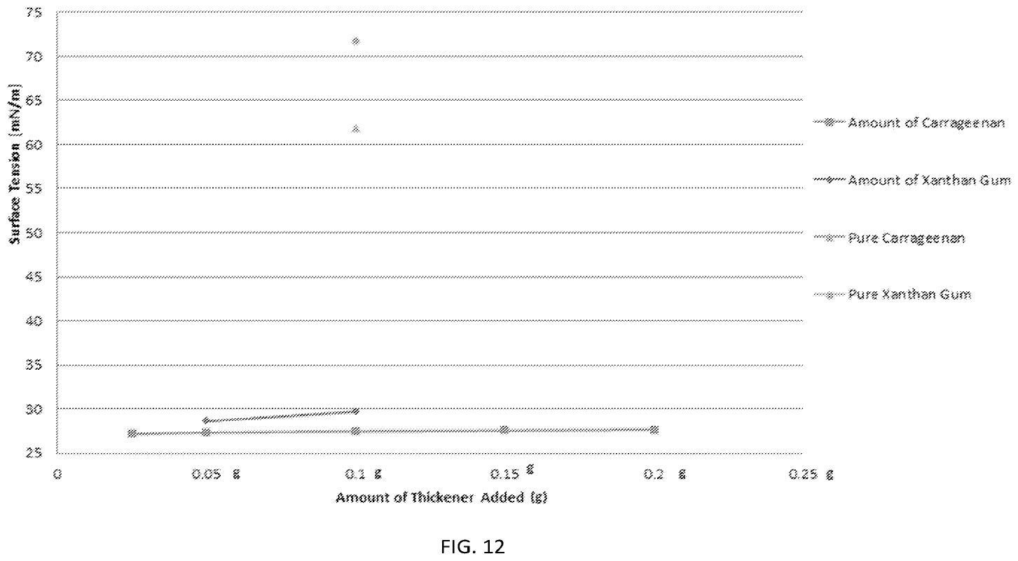

Surface tension measurements were conducted on foams formed from 20 mL of various aqueous solutions containing 5.5% w/v silk fibroin and 0.5% w/v glucoside in the presence of various amount of carrageenan or xanthan gum at 0 g, 0.05 g (0.25% w/v), 0.1 g (0.5% w/v), 0.15 g (0.75% w/v), 0.2 g (1.0% w/v), and 0.25 g (1.25% w/v), pure carrageenan solution, pure xanthan gum solution. The adoption of silk fibroin/glucoside at the air-water interface caused a significant reduction in the surface tension. The surface tension of silk fibroin/glucoside solution were increased slightly for both xanthan gum and carrageenan. More increasement of the surface tension was caused by xanthan gum than caused by carrageenan. Pure xanthan gum have no effects on reducing pure water surface tension. Pure carrageenan has a slight effect on reducing pure water surface tension from 72 mN/m to 61 mN/m (See FIG. 12). Despite the slight increasement in surface tension, no impact was observed to the foam quality and stability of the foam system stabilized by 5.5% w/v silk fibroin and 0.5% w/v glucoside.

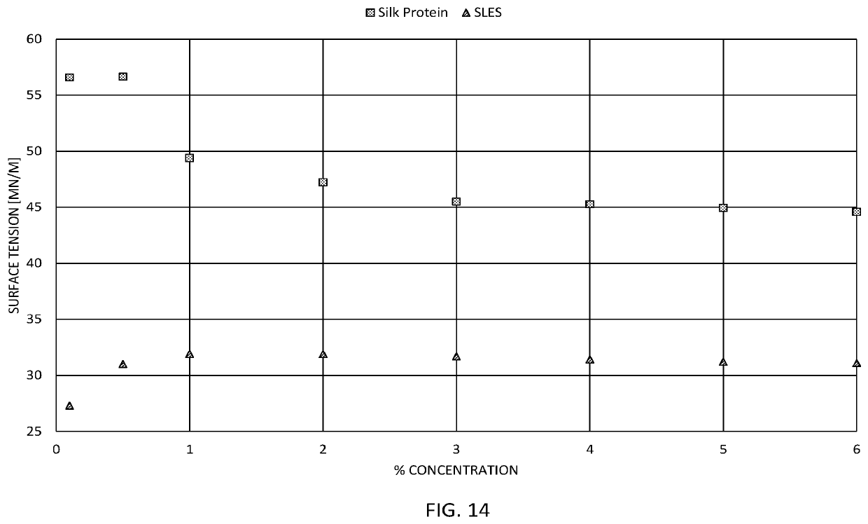

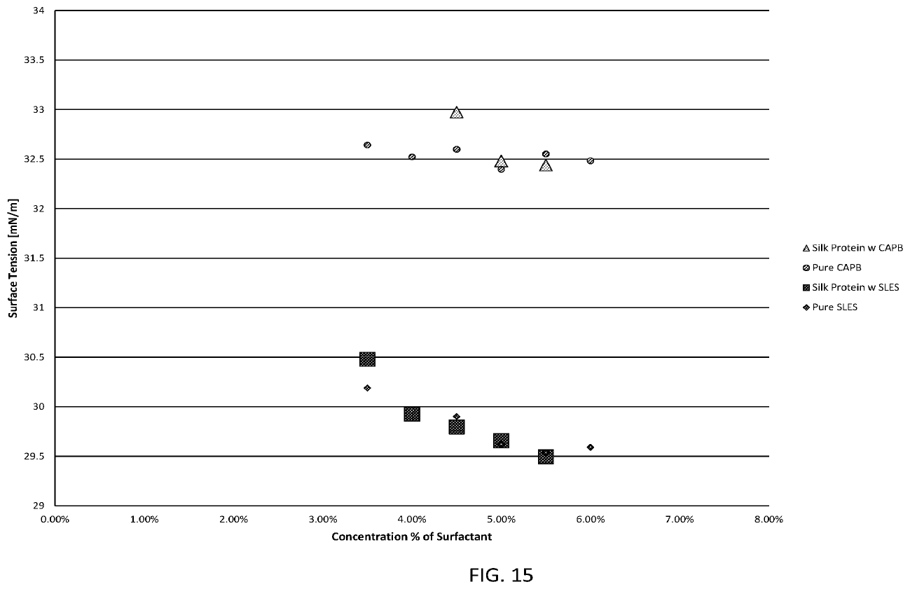

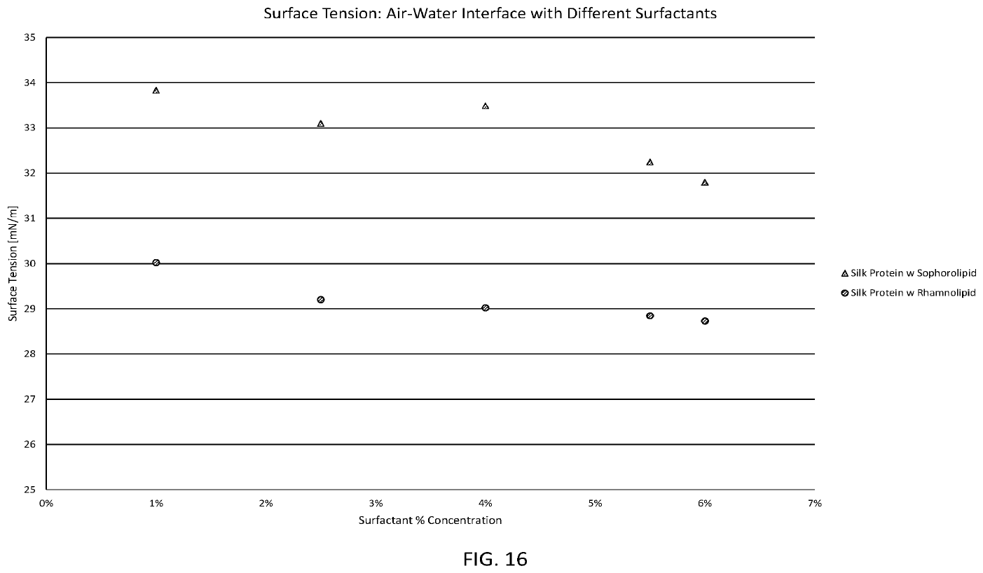

Surface tensions were measured for various combinations of the blend of varying decreasing amount of silk fibroin protein fragment at 6.0 wt. %, 5.0 wt. %, 4.0 wt. %, 3.0 wt. %, 2.0 wt. %, 1.0 wt. %, 0 wt. % with various surfactant with increasing amounts at 0 wt. %, 1.0 wt. %, 2.0 wt. %, 3.0 wt. %, 4.0 wt. %, 5.0 wt. %, 6.0 wt. % including system of silk protein and SLES, silk protein in combination with SLES and CAPB, and silk protein in combination with sophorolipid and rhamnolipid. The testing results are summarized in FIGS. 14-16.

### Example 6C. Bulk Rheology Measurement

### Impact of Thickeners on Surface Tension and Foaming

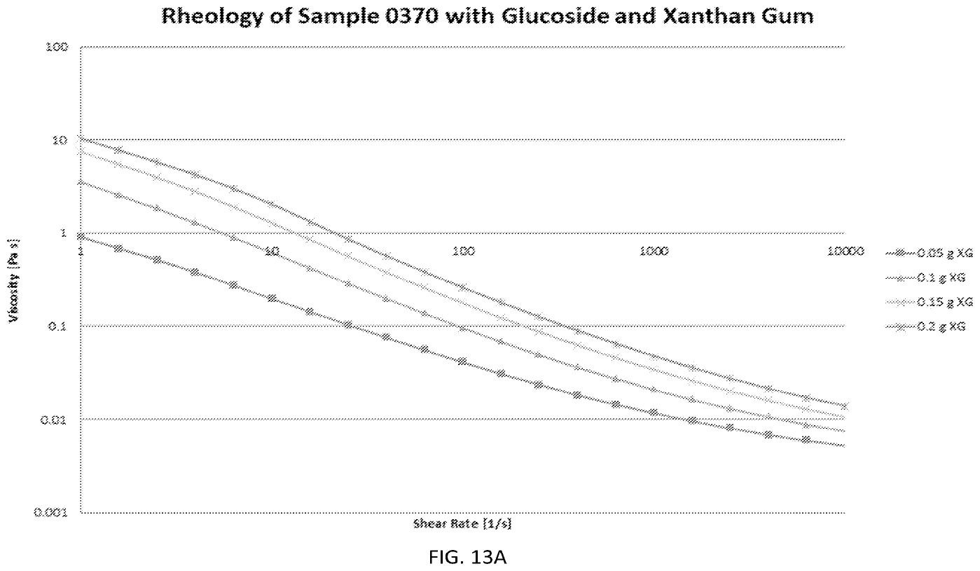

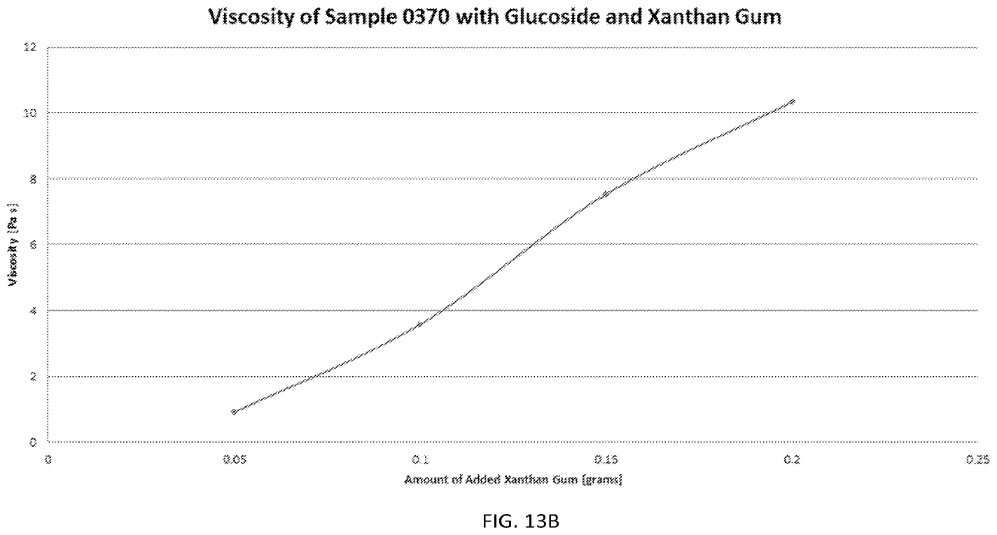

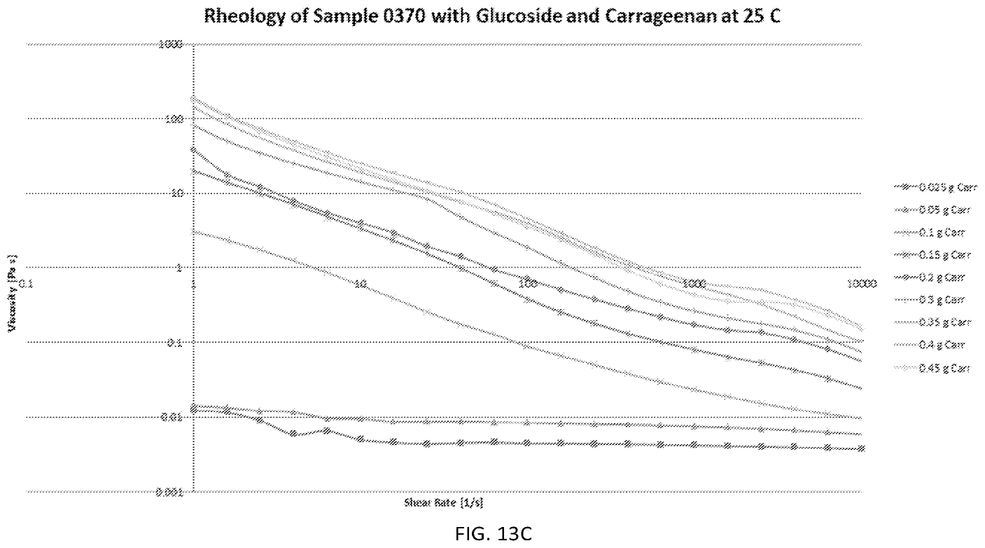

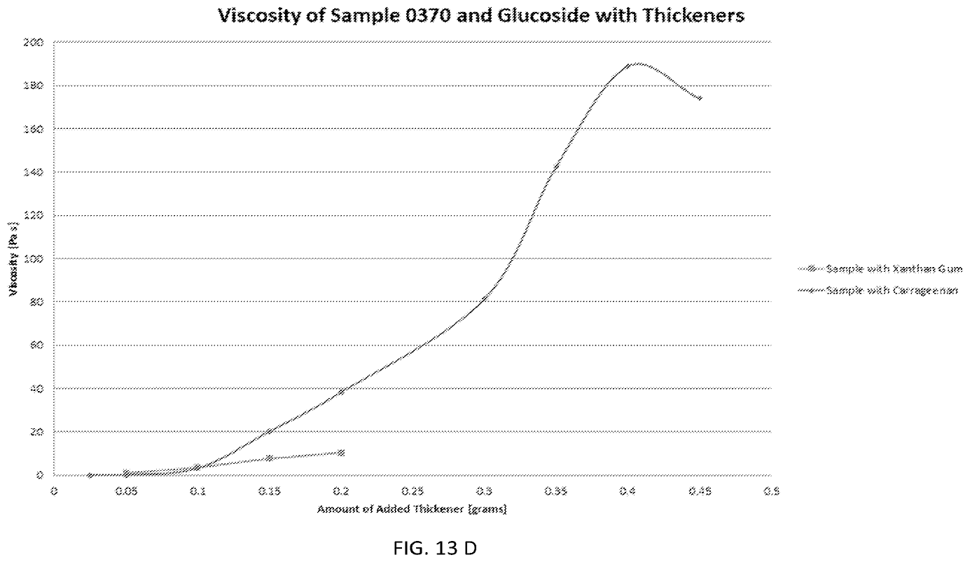

Two thickeners, xanthan gum and carrageenan were evaluated to enhance the viscosity of the silk fibroin protein fragments and caprylyl/capryl glucoside surfactant stabilized foam compositions. The vials containing the foams were allow to stand over a period of time of 45 minutes. The foam stability was then measured by monitoring the foam volume over a period of 45 minutes. The foam volume was recorded at time interval of 0, 5, 15, 30 and 45 minutes. The testing results on viscosities are summarized in FIGS. 13A-D, 17A-B and 19. Both xanthan gum and carrageenan enhance viscosity but carrageenan seems to build viscosity more effectively than xanthan gum. However, the flow behavior of the foam system having carrageenan thickener was not the same as xanthan gum.

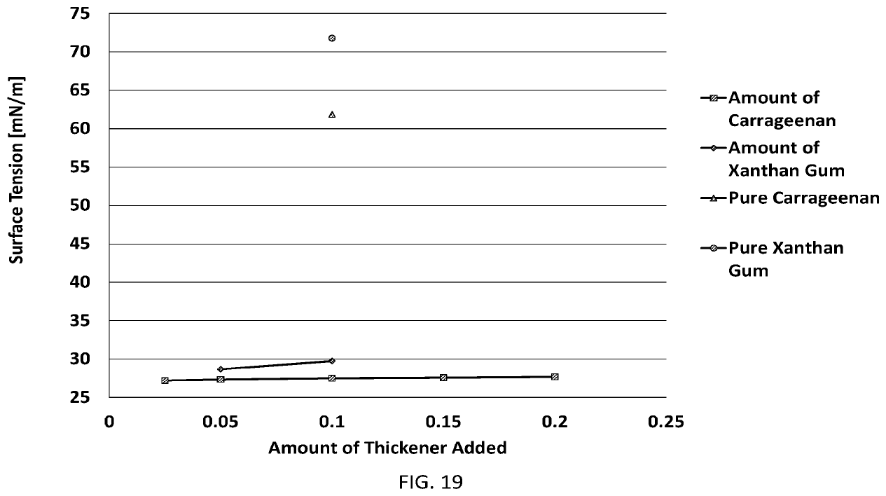

Personal care products developed from the silk fibroin protein fragments/glucoside and any of the biopolymers still need to lower surface tension at the air-water interface. In order to explore this surface tension, foaming test measurements were carried out for the silk fibroin protein fragments/glucoside blend in the presence of carrageenan or xanthan gum. The blend of 5.5 wt. % silk fibroin protein fragments with 0.5 wt. % glucoside without any thickener has a surface tension of 26.4816 mN/m. The addition of 1.0 wt. % of the both thickeners slightly increases surface tension. However, the xanthan gum increases the surface tension of the mixture more than carrageenan. (FIG. 19).

The foam test, shown in FIGS. 11A-E shows a comparison of the silk protein at 5.5% and glucoside at 0.5% without any thickener, and then the same combination with two different thickeners, carrageenan and xanthan gum. In spite of the slight enhancement of surface tension, the foam quality and stability are not impacted much and looks similar to the protein and glucoside sample. As time increases the stability of the bubbles slightly decrease with all the samples. All samples are relatively stable after forty five minutes which is noticeable in the denseness and smaller bubble size.

### G′, G″ Frequency Response

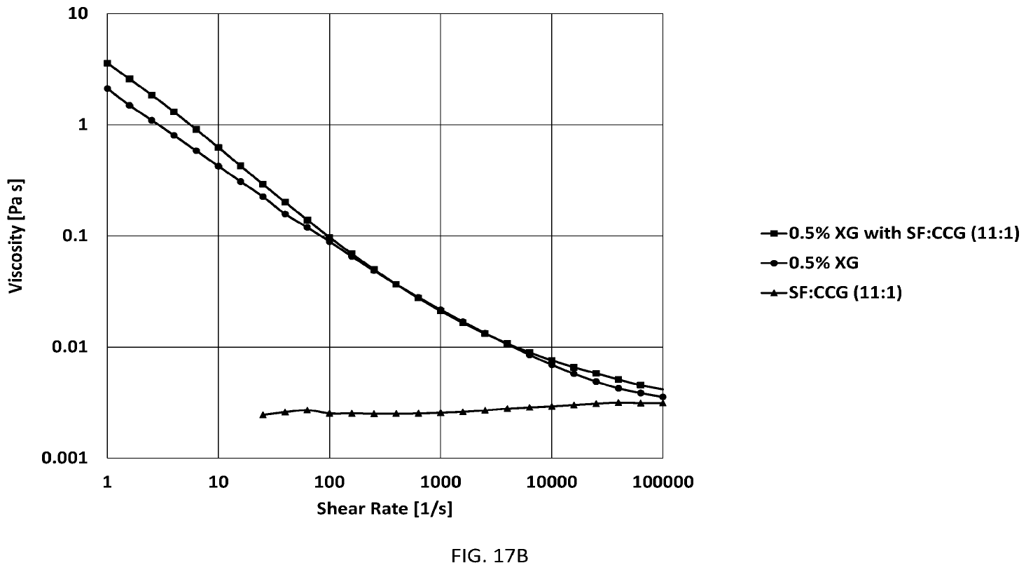

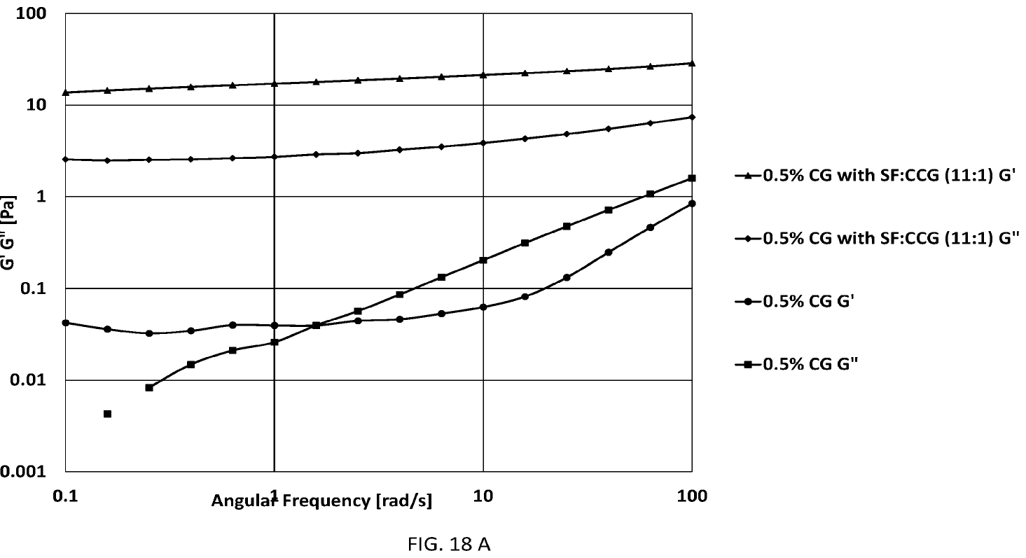

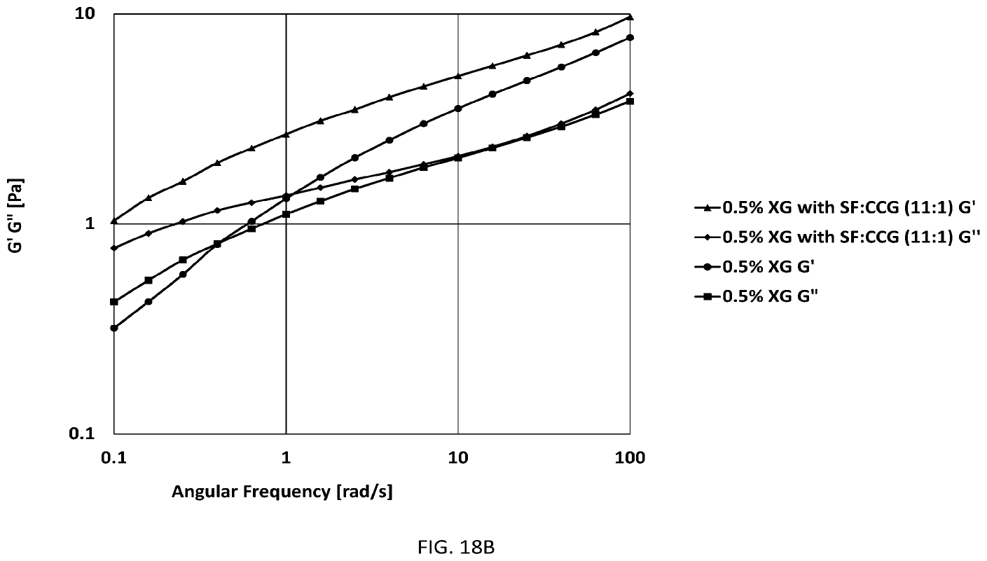

The frequency response of the silk protein and glucoside with 0.1 gram added carrageenan or xanthan gum is shown in FIG. 18a and FIG. 18b. The carrageenan in presence of silk protein and glucoside clearly shows an almost frequency independent response of G′ and G″ with the elastic modulus G′ dominating. This is a signature of an almost classical gel. In the absence of protein and glucoside carrageenan at the same concentration is mostly G″ dominated with a cross-over at low frequencies. With Xanthan gum, in presence of protein and glucoside, although G′ dominates over G″, they have a frequency dependence indicating a possible cross-over at long times (low frequencies). Additionally, the Xanthan gum in the absence of protein and glucoside exhibits a very similar frequency response with G′, still dominating over G″ and a cross-over at slightly higher frequency.

Carrageenan in combination with silk protein and glucoside almost resemble classical gel because of its frequency independent response of G′ and G″ with G′ dominating.

Xanthan gum in combination with silk protein and glucoside displays a frequency dependence despite G′ dominating G″ because of a possible cross-over and low frequencies.

The difference in rheological behavior of carrageenan and xanthan gum indicates the formation of different microstructures. The carrageenan potentially forms a common network with the protein/glucoside system giving rise to strong gel-like behaviour as indicated by the almost frequency independent response of G′ and G″. Xanthan gum however does not seem to vary much in terms of its response in the presence of protein/glucoside. This may indicate that the xanthan is actually building viscosity independently through self-assembly and H-bonding. The slight variation maybe due to some electrostatic interactions between the protein and xanthan. The difference in formed microstructures and rheology may have an impact on texture/sensory performance for a cleansing product or shampoo.

Both xanthan gum and carrageenan enhance viscosity. Both biopolymers in combination with blend of silk protein and glucoside exhibited strong Non-Newtonian behavior with strong shear thinning. The biopolymers having strong shear thinning is a benefit due to shear thinning being a highly desirable attribute in personal care products resulting in easy dispensing and spreading.

### Example 7. Combing Test

Dia-Stron MTT175 is a miniature Tensile Tester for measuring different properties of hair tresses including combing, volume, friction, curl compression, and bending. Combing test is the measurement of hair manageability by quantitative measuring combing force with unit “gmf”. Hair combing properties correlate well with subjective attributes including “ease of combing” and “detangling”. The compatibility of hair after treatment with hair care composition provides an indicator if the effectiveness of the treatment is sufficient. The smaller the numeric value of the combing force, the better the performance of the treatment.

The silk protein and glucoside formulations due to its synergistic surface tension reduction capability and corresponding effect on foam quality and stability offers a highly promising route to design cleansing products and shampoos. However, the products will require effective viscosity build. To keep with the requirement of fully sustainable/natural products, two biopolymers-xanthan gum and carrageenan were explored for their viscosity build capability.

Four testing samples were prepared including: (Test 1) 20 mL of aqueous solution containing 5.5 wt. % silk protein and 0.5 wt. % glucoside without thickeners; (Test 2) 20 mL of aqueous solution containing 5.5 wt. % silk protein and 0.5 wt. % glucoside and 0.2 g xanthan gum (1.0 wt. %); (Test 3) 20 mL of aqueous solution containing 5.5 wt. % silk protein and 0.5 wt. % glucoside and 0.2 g carrageenan (1.0 wt. %); (Test 4) 20 mL of aqueous solution containing 14 wt. % SLES with 2 wt. % CAPB solution. The combing performance of the testing samples were performed on Dia-Stron MTT175. The results are summarized in Table 17 below.

TABLE 17
Combing Testing Results

| Friction Force | Test 1 | Test 2 | Test 3 | Test 4 |
|---|---|---|---|---|
| measurement | (gmf) | (gmf) | (gmf) | (gmf) |
| Hair Treated | 176.03 | 212.77 | 384 | 67.53 |
| With Test | | | | |
| Samples | | | | |
| Hair Post Rinse | 716.6 | 557.13 | 583.4 | 439.83 |

Since the silk surfactant blend combined with xanthan gum has lower post-rinse combing force of 557.13 gmf as compared with 583.4 gmf for silk surfactant blend combined with carrageenan, the xanthan gum containing composition is more effective at detangling than the carrageenan modified composition. The addition of thickening agent xanthan gum and carrageenan to the silk surfactant blend reduced the combing force quite significantly as compared with silk surfactant blend compositions without carrageenan and xanthan gum, lower from 716.6 gmf (without) to 557.13 gmf (xanthan gum) and 583.4 gmf (carrageenan).

Therefore, treating hair with composition containing silk surfactant blend compositions combined with carrageenan or xanthan gum has significant positive effect for conditioning and a better management of the hair.

### Example 8. Preparation of Powder of Silk Fibroin Protein Fragments (SPF Powder)

### Example 8a. Freeze Drying Process

Each of the 650 mL of aqueous solution of low-MW and mid-MW silk fibroin protein fragments as prepared above was added to a 1 L round bottom glass bottle. The two bottles loaded with silk solutions were placed inside a freezer and were allowed stay inside the freezer overnight to provide fully frozen silk solutions. The two bottles containing frozen silk solutions were removed from the freezer. The bottles were left open and the openings were covered with Kimwipe paper tissues and were placed inside a lyophilizer. The pressure inside the lyophilizer is reduced to 0.02 mbar. The collector temperature was set at −65° C. After 24 hours of lyophilization, the two bottles were removed from the lyophilizer and were immediately cap to avoid the contacting the dried silk solid with moisture. The coarse powders immediately from the lyophilzation were grinded with a mortar and pestle to produce fine powders of silk fibroin protein fragments with even side distribution. The further grinding/processing may be performed to produce silk solid particle with desired particle size.

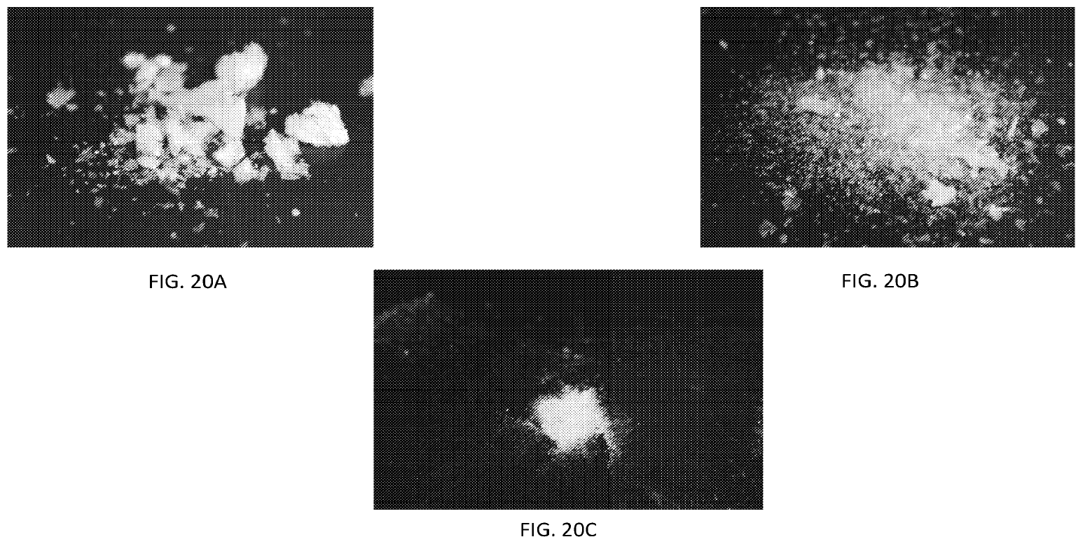

The coarse solids of low-MW silk was very easy to break down using the mortar and pestle, resulting in a very fine powder. As it became smaller, the lyophilized silk revealed a lamellar-looking appearance (approximately a couple of millimeters in length and width, but extremely thin, almost see-through). These small particles are somewhat similar to mica, in the sense that they are very thin sheets that shimmer in the light (See FIGS. 20A-C).

As the solid silk were ground more and the particle size was reduced, the powder lost its shimmer. Based on the appearance and the way it tends to fly at the slightest air movement, the particle size can be between a few microns and a few hundred microns.

The solids of mid-MW silk was much more difficult to break down with a mortar and pestle, as it did not crumble immediately upon grinding (as was the case for the low-MW solid silk). Instead, the fluffy microstructure collapses into a solid mass. A more powerful grinder may be needed (like a blender or a coffee grinder). It is possible that the mid-MW solid silk may need to be frozen below its T_{g} (glass transition temperature) in order to become more brittle and allow for grinding into very fine powders (FIG. 21).

Other silk drying methods that could be employed include, but are not limited to, spray drying, polar drying, and thin film evaporation.

### Example 8b. Thin Film Evaporation Process

Aqueous solution of Low-MW or mid-MW silk fibroin protein fragments as prepared in Example 1 was placed inside a thin film evaporator. Water was continuously removed from silk solutions inside the thin film evaporator under reduced pressure, using gentle heating, resulting in a solid of variable particle size.

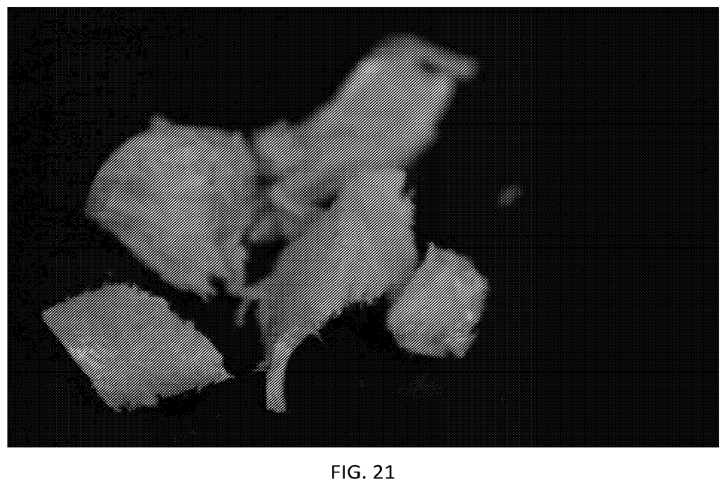

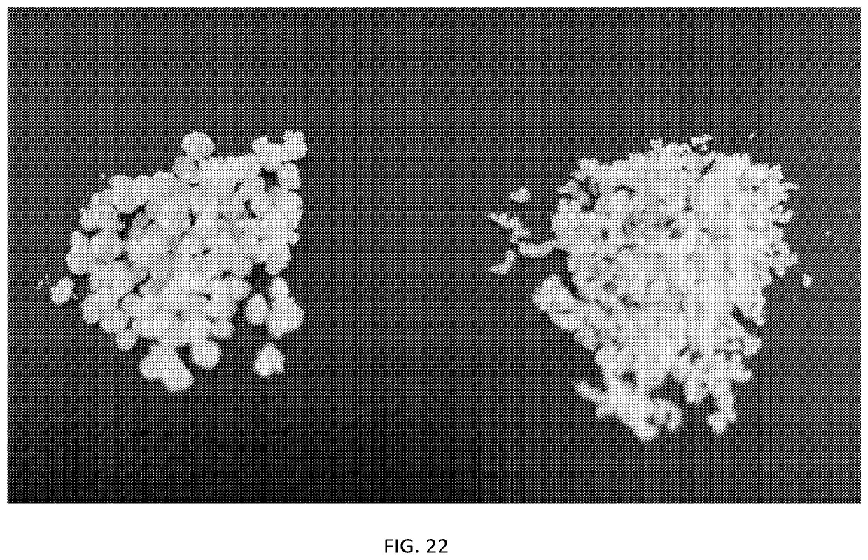

The particle size can be adjusted by varying the process parameters, such as, but not limited to pressure, temperature, rotational speed of the cylinder, thickness of the liquid film in the evaporator (FIG. 22).

### Example 8c. Microparticles Prepared by Aqueous Solution Precipitation Process

### Salt-Out Method

A 1.0 M phosphate buffer solution was prepared and the pH value was adjusted to 8. To a gently stirring silk solution of 5.0 mg/ml concentration, phosphate buffer was added in a 1:5 ratio (v/v). Samples were reacted for 5 minutes and then were placed inside a refrigerator to promote the precipitation of silk particles. The resulting silk solid suspension was then centrifuged to collect solid particles. The silk particles were washed three times with deionized water and dried to give solid particles of silk fibroin protein fragments (SPF powder).

### PVA-Assisted Method

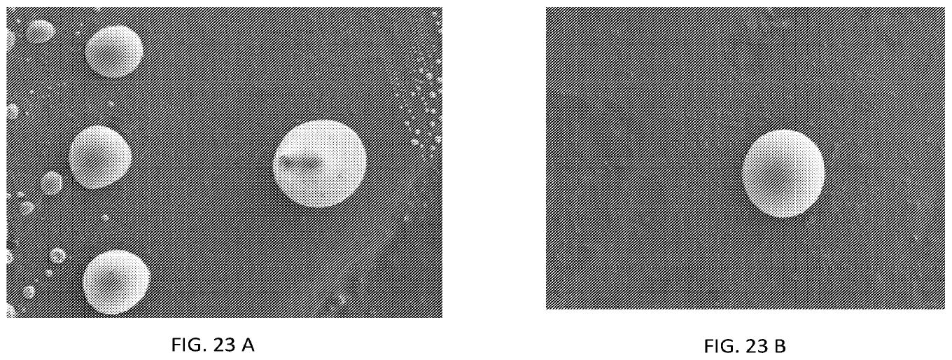

A 3.0 wt. % stock silk solution was mixed with a 5.0 wt. % solution of polyvinyl alcohol (PVA) in a 1:4 ratio (v/v). The resulting solution mixture was stirred gently for 2 hours. The solution mixture was then sonicated followed by casting to a substrate to allow formation of film. The film was reconstituted in minimal amount of D.I. water and centrifuged. The supernatant was removed and additional D.I. water was added. This process was repeated two times. After two washes, the liquid was removed from the flask to provide wet silk microparticles. Then a small volume of methanol was added to the wet microparticles in the flask (the methanol annealing). The particle suspension inside the flask was swirled. The particle suspension was then poured over a large cloth filter to isolate the microparticles (See FIG. 23A-B).

### Example 9. Application of Silk Fibroin Fragments to Fabric and Yarn Samples

### Example 9a. Dip Coating Cotton Fabric with Aqueous Silk Solution

The silk solution having a defined concentration (silk solution containing Low-MW silk fibroin fragments or silk solution containing Mid-MW silk fibroin fragments (Activated Silk™)) was added to a coating pan and was diluted with pure water to form a silk coating bath having a concentration of 0.05 wt. % fibroin protein fragments. The pH of diluted silk solution was adjusted to pH=7 with a pH adjusting agent. A piece of cotton fabric was dipped into the coating bath and immersed in the coating bath for 5 minute to allow the cotton fabric impregnated with the silk coating solution and then slowly removed and left for a few seconds on top of the coating pan to allow the excess coating solution to drip. The excess silk coating solution was then squeezed out by passing through a pad roller run at a rate of 3 meter per minute and a roller pressure setting at 50 psi. The wet dip coated fabric was dried/cured at 150° C. for 5 minute in an oven. The oven dried silk coated cotton fabric was then allowed to rest overnight at ambient conditions before testing absorbency. The silk fibroin protein fragments formed a thin layer of coating on the cotton fabric.

The pickup rate is recorded at the beginning of the experiments with an estimated rate of 51%±3%.

### Example 9b. Coating Cotton Fabric with Aqueous Silk Solution in the Presence of a Crosslinker

The silk solution having a defined concentration (silk solution containing Low-MW silk fibroin fragments or silk solution containing Mid-MW silk fibroin fragments (Activated Silk™)) was added to a coating pan and was diluted with pure water to form a silk coating bath having a concentration of 0.05 wt. % fibroin protein fragments. The pH of diluted silk solution was adjusted to pH=9 with a pH adjusting agent. To the slightly basic diluted silk coating solution, crosslinker caffeic acid was added to reach a concentration of 0.025 wt. %. After the addition of the crosslinker, the pH was readjusted to 9.

A piece of cotton fabric was dipped into the coating bath and immersed in the coating bath for 30 minute to allow the cotton fabric impregnated with the silk coating solution and then slowly removed and left for a few seconds on top of the coating pan to allow the excess coating solution to drip. The excess silk coating solution was then squeezed out by passing through a pad roller run at a rate of 3 meter per minute and a roller pressure setting at 50 psi. The wet dip coated fabric was dried/cured at 120° C. for 5 minute in an oven. The oven dried silk coated cotton fabric was then allowed to rest overnight at ambient conditions before testing absorbency

The pickup rate is recorded at the beginning of the experiments with an estimated rate of 51%±3%.

### Example 9c. Application of Silk Gel to Fabric and Yarn Samples

Essential oil fused silk gels were prepared according to Tables 18-22. Essential oil rosemary oil, lemongrass oil, lemon juice used in the table below serve as model dental/oral care active agents. Silk gels containing any of the dental/oral care agent described above may be prepared according to the formulations described in the Tables 18-22 below.

Aqueous silk fibroin-based fragment solution and essential oils are immiscible liquids. In an embodiment, to increase the fragrance of the silk fibroin-based fragment solution, without entrapping oils within the solution, the solution is mixed with the essential oil with the use of a stir bar. The stir bar is rotated at a speed such that some turbulence is observed in the mixture, thus causing contact between the fragrant essential oil and the molecules in solution, adding a scent to the solution. Before casting of product from the solution, mixing may be stopped and the oil allowed to separate to the top of the solution. Dispensing from the bottom fraction of the solution into the final product allows for fragrance without visible essential oil within the final product.

Alternatively, the silk fibroin-based solution and essential oil can be combined with or without additional ingredients and/or an emulsifier to create a composition containing both ingredients.

In an embodiment, mixing of the solution as described above can reduce gelation time if the solution is used to create a gel formulation.

Silk gels with essential oils were prepared by diluting a silk solution of the present disclosure to 2%. Vitamin C was added to the solution and allowed to dissolve. The essential oil was added, stirred and dissolved. The solution was aliquot into jars. Gels of the present disclosure can be made with ascorbyl glucoside at concentrations of about 0.67% to about 15% w/v.

Silk gel with Rosemary Essential Oil (water, silk, ascorbyl glucoside, rosemary essential oil) was collected on a tip and applied to half the length of 2 pieces of 400 μm tencel yarn. One sample was then wetted with about 0.3 mL alcohol.

Samples L1, L2, L3, L4, L5, Jar 2, R1, RO-1 and RO-2 were silk gels infused with various fragrance. Samples L1-5 contained a form of lemon juice. Samples L1 and L4 had juice directly from a lemon while samples L2, L3 and L5 contained lemon juice from a plastic lemon container. Samples L4 and L5 did not have vitamin C while all others did. All samples gelled showing that lemon juice can create gel on its own. Sample Jar 2 contained lemon grass oil which formed an albumen like substance when initially added. This sample also had vitamin C but gelation time was significantly quicker than with other vitamin C samples. Sample R1 contained rosemary oil, which seemed to be soluble, as well as vitamin C. Samples RO-1 and RO-2 contained rose oil while only RO-1 had vitamin C. In both cases the rose oil was immiscible and visible as yellow bubbles.

Both lemon juice types in the samples were able to cause gelation without the presence of vitamin C. This occurred in the same number of days as with vitamin C. The lemongrass oil was able to decrease the number of days to gelation to 2-3 days. All additives appeared soluble other than lemongrass oil and rose oil. Rose oil remained in yellow bubbles while the lemongrass oil was partially soluble and formed an albumen like chunk. In an embodiment, oils that are not fully soluble, can still be suspended within the gel as an additive.

TABLE 18
Gel Samples - Silk gel formulations including additives, concentration of silk
and additive, gelation conditions and gelation times.

| | mL 2% | Mass | Ratio | | Amount | | | |
|---|---|---|---|---|---|---|---|---|
| Sample | silk | Vit C | silk: | | of | | Temp/ | Days to |
| Name | solution | (g) | VitC | Additive | additive | | Treatment | Gelation |
| Ll | 10 | 0.04 | 5:01 | Lemon | 300 | uL | RT | 6 |
| L2 | 10 | 0.04 | 5:01 | Lemon Juice | 300 | uL | RT | 6 |
| L3 | 10 | 0.04 | 5:01 | Lemon Juice | 1000 | uL | RT | 5 |
| L4 | 10 | 0 | None | Lemon | 300 | uL | RT | 6 |
| L5 | 10 | 0 | None | Lemon Juice | 300 | uL | RT | 7 |
| Jar 1 | 20 | 0.08 | 5:01 | Lemon Juice | 2000 | uL | RT | 5-7 |
| Jar 2 | 5 | 0.02 | 5:01 | Lemongrass | 1 | drop | RT | 2-3 |
| | | | | Oil | | | | |
| R-1 | 10 | 0.04 | 5:01 | Rosemary | 1 | drop | RT | 7 |
| | | | | Oil | | | | |
| T-1 | 10 | 0.04 | 5:01 | None | None | | RT/Tube | 7 |
| RO-1 | 10 | 0.04 | 5:01 | Rose Oil | 1 | drop | RT | 6 |
| RO-2 | 10 | None | None | Rose Oil | 1 | drop | RT | None |

TABLE 19
Lemongrass Gel

| | |
|---|---|
| % Silk Solution | 2% |
| Quantity Vitamin C | 100 mg/15 mL solution |
| Quantity Lemongrass Oil | 20 μL/15 mL solution |

TABLE 20
Rosemary Gel

| | |
|---|---|
| % Silk Solution | 2% |
| Quantity Vitamin C | 100 mg/15 mL solution |
| Quantity Rosemary Oil | 20 μL/50 mL solution |

TABLE 21
Lemongrass Gel (50 mL)

| | |
|---|---|
| % Silk Solution (60 minute boil, 25 kDA) | 2% |
| Quantity Vitamin C (ascorbyl glucoside) | 12.82 mg/mL solution |
| | (641 mg total) |
| Quantity Lemongrass Oil | 1.33 μL/mL solution |
| pH | 4 |

TABLE 22
Rosemary Gel (50 mL)

| | |
|---|---|
| % Silk Solution (60 minute boil, 25 kDA) | 2% |
| Quantity Vitamin C (ascorbyl glucoside) | 12.82 mg/mL solution |
| | (641 mg total) |
| Quantity Rosemary Oil | 0.8 μL/mL solution |
| pH | 4 |

### Example 10: Synergistic Properties Between Soluble Silk Fibroin and Capryl Glucoside Lead to a Natural and Effective Co-Surfactant System

In response to consumer demand for more natural and sustainable personal care products, soluble silk fibroin (SF) was evaluated for its use as a natural co-surfactant. In combination with caprylyl/capryl glucoside (CCG), SF was found to outperform other commonly used commercial surfactants, demonstrating better surface tension properties, cleansing properties, and foamability than sodium laureth sulfate (SLES), cocamidopropyl betaine (CAPB), rhamnolipids and sophorolipids. The viscosity of the SF/CCG co-surfactant system was enhanced with polysaccharide rheological modifiers, resulting in an all-natural formulation with excellent performance. The co-surfactant system can be further used to formulate all-natural products such as cleansers or shampoos with similar or improved efficacy as formulations containing synthetic surfactants

In recent years the consumers had become increasingly concerned about the environmental and health consequences of utilizing synthetic, petroleum-based ingredients in products from a variety of industries such as food, fashion and personal care. This heightened awareness led to an increased demand for “clean” and “natural” ingredients and products. In the personal care industry, the consumer demand manifested into a renewed interest in natural, clean surfactants that are sustainable, biodegradable, and biocompatible, yet still provide excellent performance and efficacy. As such, an increasing number of new personal care products in the market include natural surfactants, either alone or in concert with traditional petrochemical-based surfactants.

Most biosurfactants are glycolipids synthesized by microorganisms and, similar to synthetic surfactants, they function by reducing interfacial and surface tensions. Besides their obvious advantages (e.g. higher biodegradability, superior environmental compatibility, and decreased toxicity), biosurfactants also tend to have better foaming abilities and lower critical micelle concentrations. For these reasons, biosurfactants have found use in the personal care industry, as well as health, chemical, petroleum, food and agricultural industries. Alkyl glucosides are another attractive type of surfactant because they are derived from sugars and fatty alcohols that are present in natural, renewable resources, and in addition are water soluble and display an increased adherence towards strong ionic surfactants. Proteins have also gained considerable interest as sustainable, natural surfactants for personal care products due to their structure, surface activity and charge interaction effects.

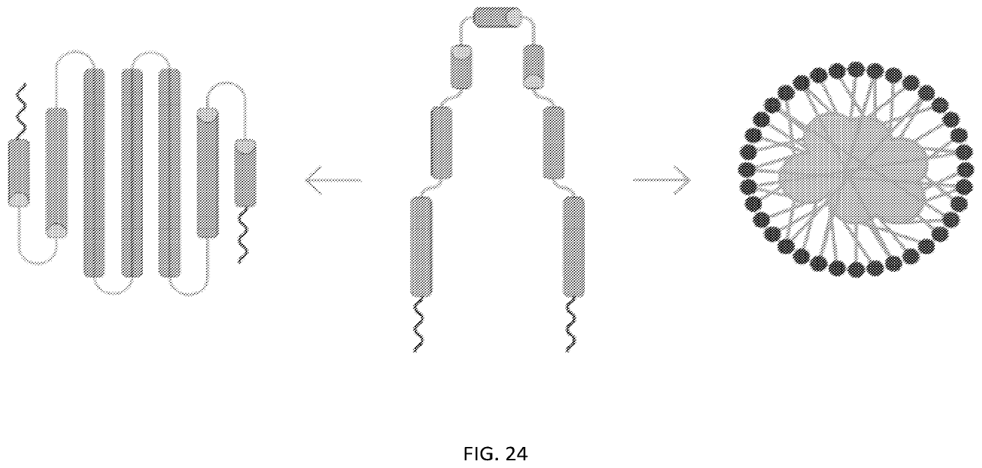

Silk fibroin protein is of particular interest for use as a surfactant as it displays structural features similar to surfactants already used in industry. Silk fibroin is made up of a heavy chain and a light chain connected by a disulfide linkage and an associated unit of P25 glycoprotein. The heavy chain consists of highly hydrophobic domains interspersed with amorphous regions containing negatively charged, hydrophilic residues. This unique structure, with alternating hydrophilic and hydrophobic domains, makes silk fibroin a natural amphiphilic multiblock copolymer, which drives the formation micellar structures exhibiting the typical core-shell architecture, as shown schematically in FIG. 24. The hydrophobic core contains mainly the crystalline and amorphous domains, while the hydrophilic shell consists of the terminal domains of silk fibroin. In solution, the micelles only interact loosely, but the amphiphilic structure allows the silk fibroin to self-assemble and form stable viscoelastic films at air-water or oil-water interfaces, preventing droplets or bubbles from coalescing while also preventing macroscopic phase separation and increasing the stability of foams and emulsions. For these reasons, silk fibroin has been explored of as a sustainable surfactant and biocompatible emulsion stabilizer in the personal care industry.

The amphipathic structure of silk fibroin can also facilitate the interaction with other amphiphilic molecules (like alkyl glucosides) to further enhance the surfactant properties of the binary complex. In this Example, the surfactant-associated properties of a soluble and stable form of silk fibroin (Activated Silk 1004-LS) were investigated alone and in combination with a model alkyl glucoside (caprylyl/capric glucoside, CCG). Specifically, the synergies of this binary system were evaluated on the water-air interface surface tension, foamability, foam stability and cleansing potential. The feasibility of formulating this mixture with rheological modifiers was also examined.

Materials and Methods

Materials

(Activated Silk 1004-LS), a stable form of soluble silk fibroin (SF) was used throughout this study (Evolved by Nature, Medford, Mass.). All surface activity, surface tension or elasticity measurements were carried out at the same total surfactant concentration. Caprylyl/capryl glucoside (ORAMIX™ CG 110) was acquired from Seppic (Fairfield, N.J.). Sodium laureth sulfate (SLES, Sulfochem™ ES-1 Surfactant 25.5%) and cocamidopropyl betaine (CAPB, Chembe-taine™ ACB Surfactant, 36%) were provided by Lubrizol (Cleveland, Ohio) as well as a rhamnolipid (Rheance One, 48.7%) and a sophorolipid solution (Rewoferm SL One, 40%) which were both provided by Evonik Industries (Essen, Germany). Two thickeners were also used in this study, Xanthan Gum manufactured from Tokyo Chemical Industry Co., LTD (Tokyo, Japan) and k-carrageenan from Sigma Life Sciences (Milwaukee, Wis.). The materials used for the detergency studies were virgin straight medium brown hair tresses (International Hair Importers, ca. 1.5 g each), Artificial Sebum (Pickering Laboratories, ASTM D4256, not stabilized), and n-Hexane (VWR).

Sample Preparation

All samples were prepared in DI water with a total surfactant concentration of 6 wt. %.

Various ratios of soluble silk fibroin and co-surfactants totaling 6 wt. % were formulated in glass vials, shaken lightly, and left for twenty-four hours at 4° C. to ensure homogeneity before testing.

Thickened formulations of SF:CCG co-surfactant solution were also prepared via addition of xanthan gum (XG) or carrageenan gum (CG). The samples were prepared by sequentially adding SF, CCG, DI water and either XG or CG. The thickener was added to the formulation at 45° C. and slowly mixed until completely homogeneous. Samples were then left for twenty-four hours at 4° C. before testing. All samples were prepared at a total volume of 20 mL and used without dilution.

Mechanical Rheology

TA instrument DHR-3 rheometer (TA Instruments, Delaware, USA) was used in this study to measure flow curves and storage modulus (G′) and loss modulus (G″) frequency response. A 25 mm parallel plate was used for each experiment with a Peltier plate controlling the temperature at 25° C. Before the frequency sweep to extrapolate G′ and G″, an amplitude sweep was done to determine the appropriate strain percent from the linear viscoelastic region of each sample.

Du Noüy Ring Method

Surface tension at the air-water interface was tested at 20° C. with the Du Noüy ring technique on the Attension Sigma 701 Tensiometer (Biolin Scientific, Gothenburg, Sweden). A small vessel was used for each test with 20 mL of sample and wait time of three hours was used before the du Noüy ring began to measure the surface tension.

Foam Test

The foam test was performed by taping samples together and shaking uniformly for ten seconds. The foam build-up was recorded at T=0, 5, 15, 30 and 45 minutes.

Sebum Removal Test

The initial weights of the virgin straight medium brown hair tresses (approximately 1.5 g each) were recorded. Artificial sebum solution (1 mL; 30% in hexane) was applied to each tress and combed through uniformly. The tresses were allowed to dry for an hour at RT to allow the hexane to evaporate and then reweighed for their soiled weight. In order to test the efficacy of each surfactant solution, each tress was rinsed with cold tap water for 10 seconds before applying 1 mL of surfactant solution and lathering for 30 seconds. After lathering, the tress was again rinsed for 10 seconds and hung to air dry overnight. After drying, the final weight of the washed hair tress was recorded, and the percentage of sebum removed from the tress was calculated. N=2 tresses were tested for each surfactant solution.

Results and Discussion

Surface Activity: Surface Tension at Air-Water Interface & Impact on Foaming

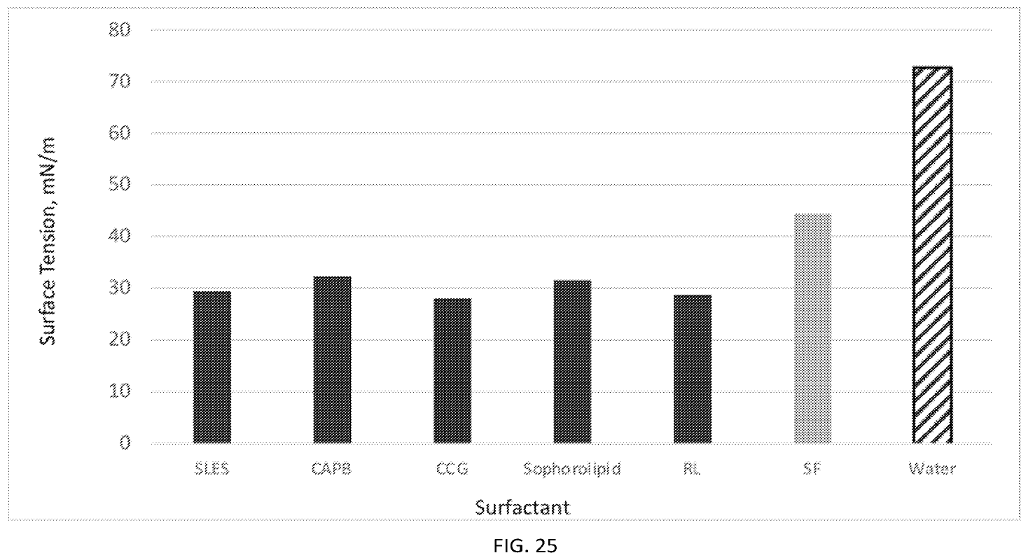

Surface activity of surfactants is a critical parameter in the performance of personal care products as the ability to lower the surface tension at the air-water interface allows for enhanced foam quality, foam stability and cleansing efficacy. To evaluate the surface activity of soluble silk fibroin (SF), the air-water interface tension of SF was compared to two traditional surfactants used in personal care, sodium laureth sulfate (SLES) and cocamidopropyl betaine (CAPB) and three bio-based surfactants—rhamnolipids, sophorolipids and caprylyl/capryl glucoside (CCG). SF displayed tensioactive properties and reduced the surface tension of water from 72.8 mN/m to 44.5 mN/m. While not wishing to be bound by any particular theory, this result may be due to its amphiphilic structure. However, the surface tension reduction with SF is not as significant compared with the other surfactants investigated (FIG. 25).

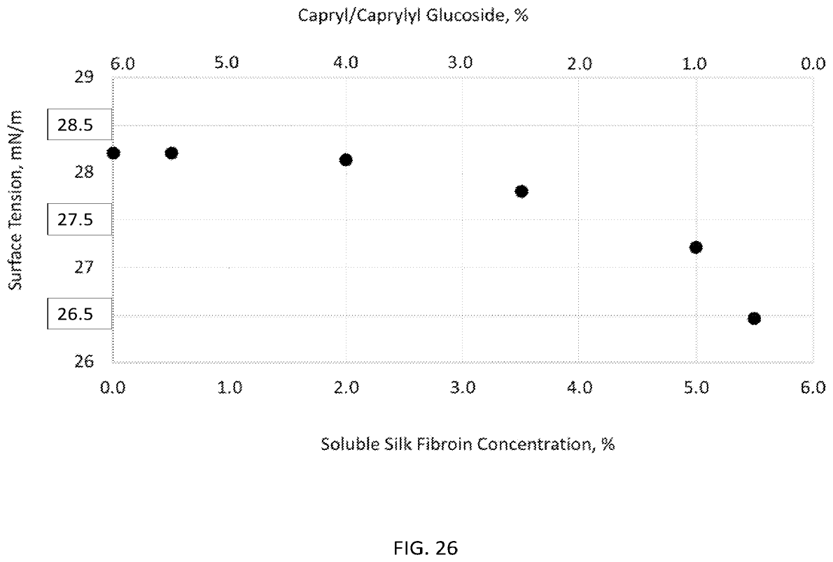

Silk fibroin was tested in mixtures with other co-surfactants. Due to its amphipathic structure, it was hypothesized that silk fibroin would have the ability to interact with other amphiphilic molecules (like alkyl glucosides) and give rise to interesting surface properties for the binary complex. Indeed, when combined with CCG, soluble silk fibroin exhibited a nonlinear, inverse dependence between the silk fraction and the surface tension. That is, as SF fraction increases, the water/air interface surface tension decreases. As shown in FIG. 26, the surface tension of the system starts at 28.20 mN/m (pure CCG) and gradually decreases as the fraction of SF increases, reaching a minimum of 26.5 mN/m at a 11:1 ratio of SF:CCG. This surface tension reduction is counter-intuitive to what one would expect based on the individual contributions of the two surfactants. While not wishing to be bound by any particular theory, this result strongly suggests a synergistic interaction at the air-water interface between SF and CCG.

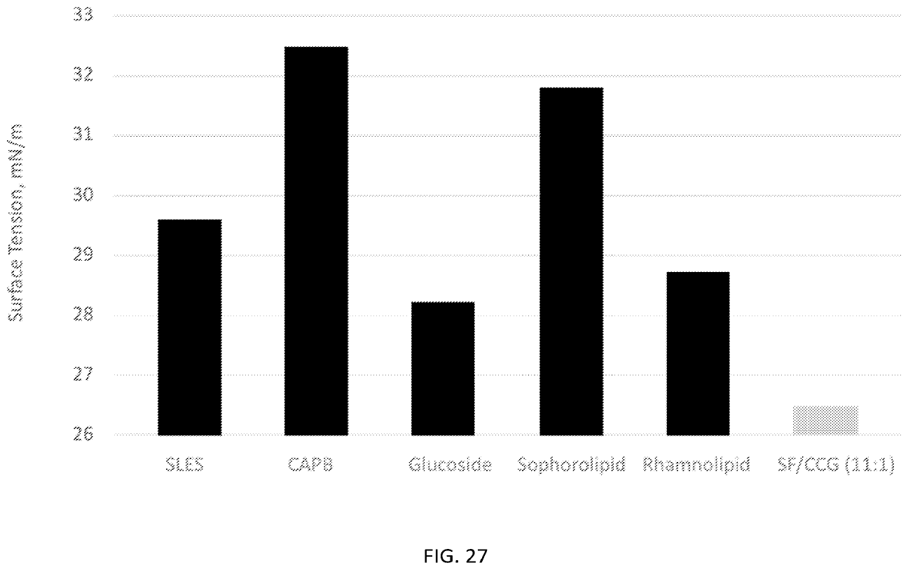

It is also worth noting that the surface tension of the 11:1 SF:CCG formulation is not only lower than the surface tension of each of the pure components, but also lower than any of the other surfactants or surfactant mixtures tested in this study, including SLES (FIG. 27). While not wishing to be bound by any particular theory, this result suggests that only a very small amount of CCG is required to bridge the silk protein structure and form a stable air-water interface, and may be accomplished via hydrophobic interactions of the CCG and SF hydrophobic domains. (FIG. 26).

Foaming Behavior

Foaming is a desirable property for surfactants in personal care applications such as shampoos and body washes as consumers usually associate it with good performance. As such, the foamability and foam stability of the green surfactants (CCG, rhamnolipids, and sophorolipids) were measured and compared to the SF/CCG (5:1) mixture.

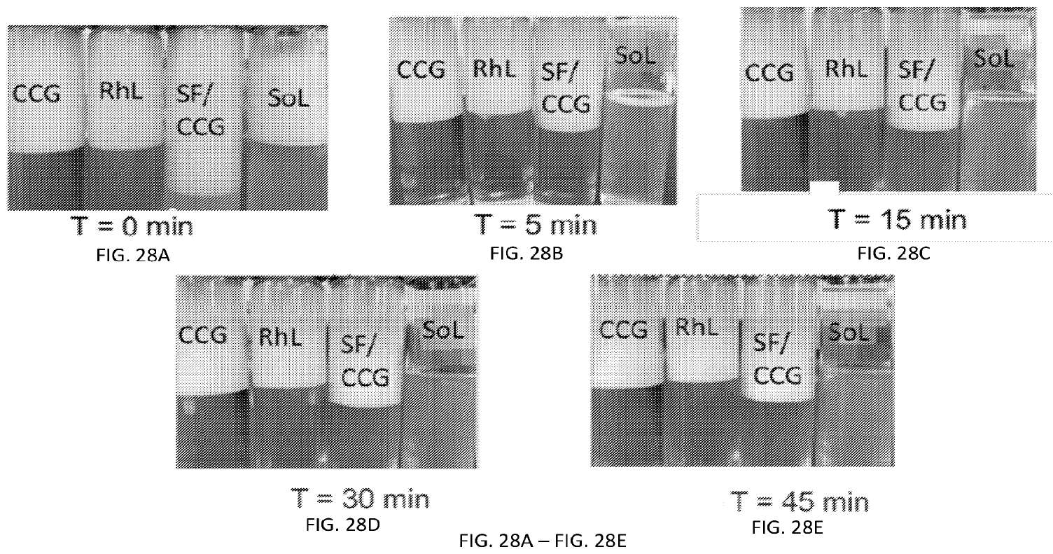

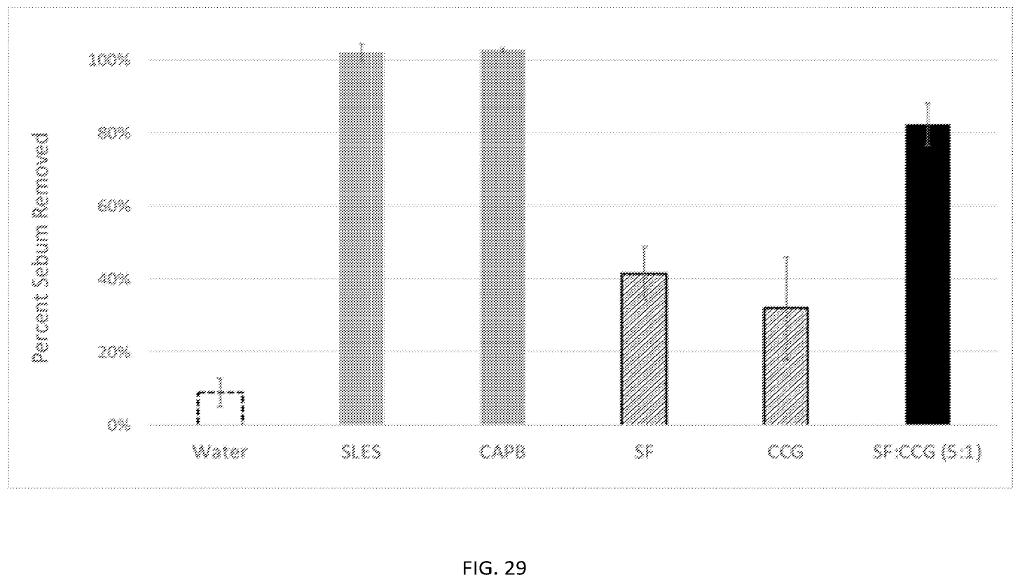

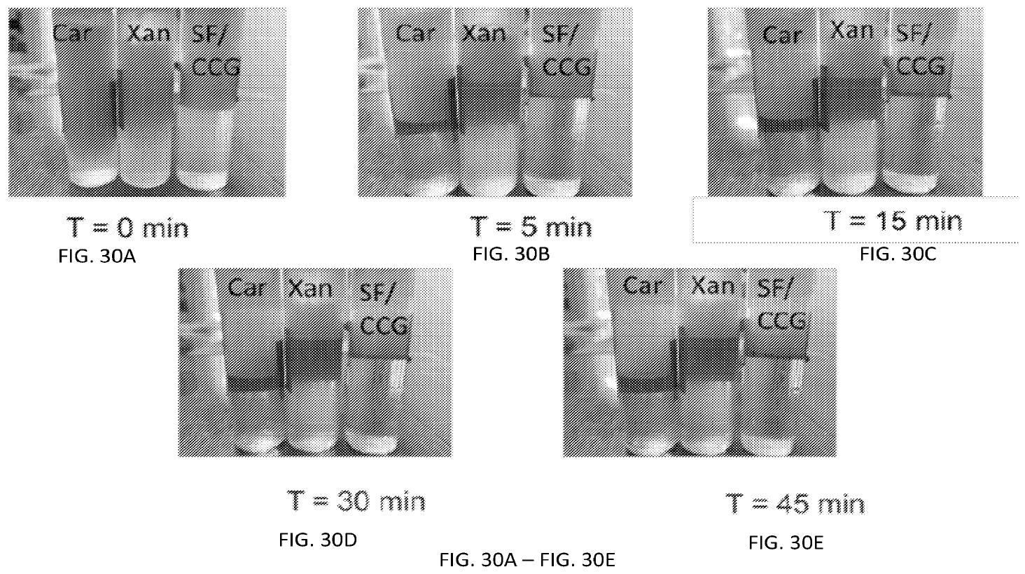

The results of the foaming test are shown in FIG. 28A-FIG. 28E. In agreement with the surface tension data, the SF/CCG produced the most foam (FIG. 28A). Over time the foam volume decreased for all samples, but the SF/CCG mixture displayed the best foam stability, retaining the largest foam volume after 45 minutes.

Sebum Removal Through Cleansing Properties

Although foamability and foam stability are important qualities contributing to customer satisfaction, cleansing performance of the surfactant is a critical attribute. As such, artificial sebum removal from hair tresses was employed as a model system to assess the efficacy of the SF:CCG system.

Results show that the lower surface tension and enhanced foamability of the SF/CCG system resulted in good cleansing performance, as evidenced by high percent removal of artificial sebum from hair tresses (FIG. 29). Furthermore, synergy between SF and CCG (resulting in lower surface tension) was also observed in the cleansing data. That is, the SF:CCG formulation removed significantly more sebum from the hair tresses than would be expected from the sum of the individual contributions of SF and CCG alone, and was shown to be an effective cleanser comparable to SLES and CAPB.

Rheology Build in Silk Protein+Glucoside Formulations

Alkyl sulphates and alkyl ether sulphates (like SLS and SLES) are commonly used in personal care, but they are sometimes linked to skin and eye irritation while there are also safety concerns associated with the possible presence of residual 1,4-dioxane in alkyl ether sulphates. Sulfate-free surfactants often have a poor foamability and they do not thicken well with common rheology modifiers. Compatibility with rheology modifiers is an important property for surfactants used in personal care given the wide use of modifiers to achieve the desired texture of the final product. As demonstrated in the sections above, SF:CCG has excellent foamability and cleansing properties. To evaluate the compatibility of the SF/CCG system with typical rheology modifiers, the effect of two biopolymers, carrageenan and xanthan gum, on the rheology of SF/CCG system was examined.

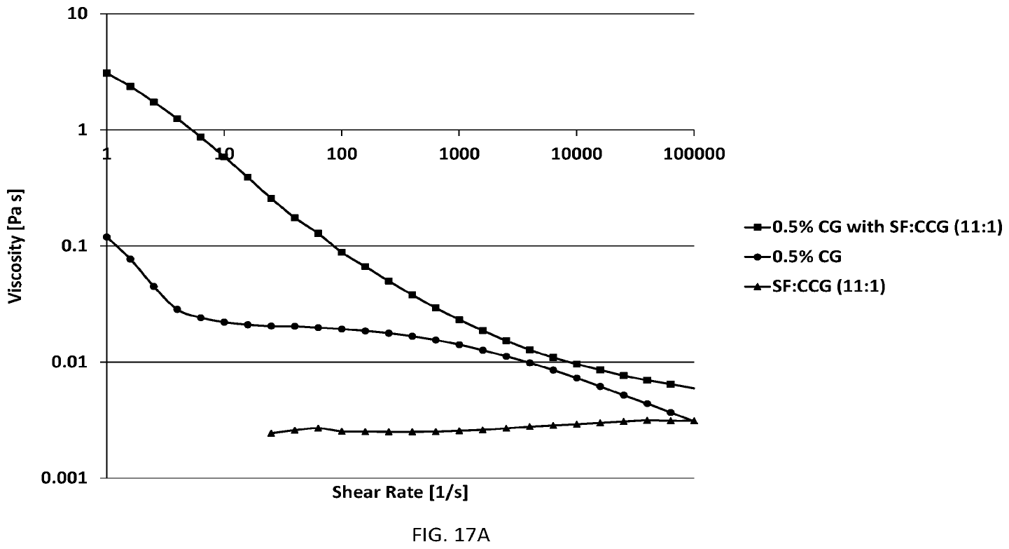

Rheology data for SF:CCG (11:1) thickened with either XG or CG is shown in FIG. 17A-FIG. 17B. FIG. 17A illustrates the flow sweep of SF:CCG (11:1) with added 0.5 wt % of CG FIG. 17A illustrates the flow sweep of SF:CCG (11:1) with added 0.5 wt % of XG.

Without thickeners, the viscosity of SF/CCG is mostly independent of the shear rate. However, addition of either XG or CG enhanced the viscosity of the solution and resulted in typical non-Newtonian behavior with a strong shear thinning character. This shear thinning behavior is a desirable attribute for personal care products because it results in easy dispensing and spreading of the formula.

The texture and sensory performance of a personal care product depends in part on the interplay of G′ and G″ frequency response. By measuring the two modulus, they can give insight on the sensorial characteristics of a product. The higher the elastic modulus, the more stiffer and elastic the perception of the product usually is. The G′ and G″ frequency response of the SF/CCG with 0.5 wt % added carrageenan or xanthan gum is shown in FIG. 18A and FIG. 18B. FIG. 18A illustrates the storage and loss modulus for SF:CCG (11:1) with added 0.5 wt % of CG. FIG. 18B illustrates the storage and loss modulus for SF:CCG (11:1) with added 0.5 wt % of XG. By itself, the behavior of carrageenan is mostly dominated by G″ with a cross-over at low frequencies. However, addition of carrageenan resulted in formation of an almost classical gel, as clearly shown by the almost frequency independent response of G′ and G″, with the elastic modulus G′ dominating.

Xanthan gum in the presence of SF/CCG shows a frequency dependence despite G′ dominating over G″. While not wishing to be bound by any particular theory, this result indicates a possible cross-over at longer times (low frequencies). Additionally, xanthan gum in the absence of SF/CCG exhibited a very similar frequency response with G′, still dominating over G″. Xanthan gum on its own exhibited a cross-over at a slightly higher frequency.

This difference in rheological behavior between carrageenan and xanthan in the presence of SF/CCG points to formation of very different underlying microstructures. The carrageenan potentially forms a common network with the protein/glucoside system giving rise to strong gel-like behavior as indicated by the almost frequency independent response of G′ and G″. This is similar to what is seen in carrageen interactions with ionic surfactants where a common structured system is formed due to interactions between carrageenan and surfactants. Xanthan gum however does not seem to vary much in terms of its response in the presence of protein/glucoside. While not wishing to be bound by any particular theory, this may indicate that the xanthan is actually building viscosity independently, through self-assembly and H-bonding. The slight variation may be due to some electrostatic interactions between SF and XG. These variations in microstructure and rheology may have implications in texture/sensory performance in a cleansing product or shampoo.

Finally, surface tension and foamability measurements were performed with SF/CCG in presence of carrageenan or xanthan. While addition of thickeners slightly increased surface tension, the foaming properties were not compromised and remained consistent with those obtain without thickeners.

The surface activity of soluble silk fibroin was found to be synergistically enhanced in the presence of small amounts of a natural alkyl glucoside surfactant (caprylyl/capryl glucoside) resulting in a surface tension at the air-water interface which is lower than that of either soluble silk fibroin or glucoside alone.

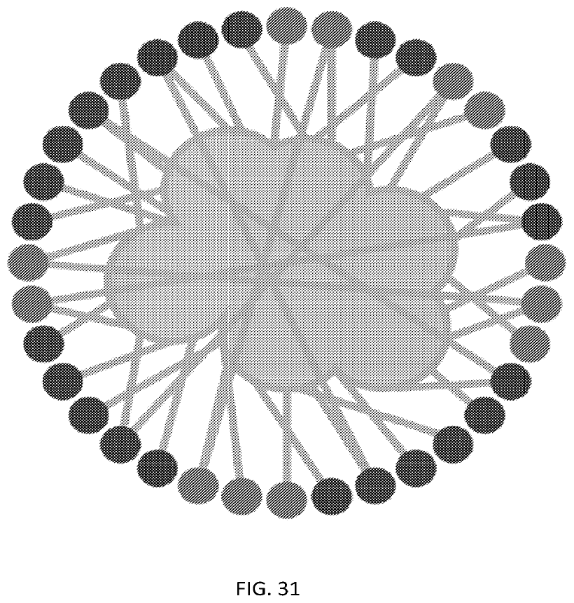

A combination of health and environmental awareness lead consumers to increasingly demand natural, renewable surfactant systems in personal care formulations. A novel combination of soluble silk fibroin and a natural alkyl glucoside (caprylyl/capryl glucoside) was compared in this study with commercially available examples of two classes of natural surfactants (rhamnolipids and sophorolipids). The study revealed the synergistic cooperativity between soluble silk fibroin and glucoside which manifested in significantly reduced air-water surface tension. This surface tension value is additionally lower than that obtained from currently utilized synthetic surfactants like SLES/CAPB. The low surface tension values obtained through combinations of silk proteins and glucoside was accompanied by formation of stable foams and enhanced sebum removal. While not wishing to be bound by any particular theory, the synergistic effect seen in this combination may be due to the hydrophobic regions of the glucoside interconnecting with the hydrophobic domains in the β-sheet structure of the silk protein which forms at the air-water interface. (FIG. 31) While a similar type of synergistic effect has been previously reported for BSA, lysozyme and biosurfactants, these proteins need to be used at high surfactant concentrations, unlike the SF/CCG co-surfactant system where only 0.5% of glucoside was needed for the optimal combination The enhanced surface activity of the silk-glucoside mixture translated into very good cleansing properties, as measured by the artificial sebum removal assay.

The rheological performance of the silk proteins was impacted through synergistic interactions with biopolymers like carrageenan. It was observed that both the flow curve and the absolute viscosity values were significantly impacted in the presence of carrageenan, with higher viscosity generation and significant non-Newtonian/shear thinning behavior evolution. This was not the case for xanthan gum where the rheology build-up was not as significant. Similar to interactions observed between carrageenan and ionic surfactants, there may potentially be an ordered composite structure being formed between the carrageen and protein possibly due to ionic and hydrophobic interactions.

These results indicate that soluble silk fibroin offers desirable properties that can be leveraged in the development of high-performance and natural personal care products. Specifically, this study demonstrates that soluble silk fibroin can significantly enhance the performance of natural/sustainable cosmetic formulations through building synergistic interactions with other natural ingredients such as sugar surfactants and biopolymer.

### Example 11: Soap and/or Shower Gel Formula

Soap Formula LHS-60 was formulated as follows:

LHS-60

| Ingredient | Wt % |
|---|---|
| Water | 86.88 |
| Caprylyl/capryl glucoside (about 60-70% active material) | 0.04 |
| Activated Silk (6% soln; low molecular weight) | 0.59 |
| Cocobetaine | 7.50 |
| Decyl glucoside | 1.80 |
| Aspen bark extract | 0.90 |
| Dermosoft anisate | 0.50 |
| Natrosol 250 HHR CS | 0.85 |
| 1,3-propanediol | 0.90 |
| 50/50 citric acid | 0.05 |

The caprylyl/capryl glucoside used in this non-limiting formulation was about 60-70% active material, which results in a ratio of silk fibroin fragments to caprylyl/capryl glucoside of about 3.5:2.5.

### Example 12: Hand Sanitizer Formula

A hand sanitizer was formulated as follows:

Hand sanitizer

| Ingredient | Wt % |
|---|---|
| Ethanol | 70.3% |
| Activated Silk (6% soln; low molecular weight) | 1.0% |
| Hydroxypropylcellulose | 0.5% |
| Water | 28.2% |

Antimicrobial efficacy against various test organisms was determined by an in vitro “time kill” test, and determined to be as follows:

| | ATCC | Log Reduction | | % Reduction | |
|---|---|---|---|---|---|
| Test Organism | Number | 15 seconds | 30 seconds | 15 seconds | 30 seconds |
| Haemophilus influenza | 33391 | >4.07 | >4.07 | >99.99 | >99.99 |
| Bacteroides fragilis | 25285 | >4.17 | >4.17 | >99.99 | >99.99 |
| Escherichia coli | 25922 | >5.14 | >5.14 | >99.999 | >99.999 |
| Klebsiella pneumonia | 13883 | >5.23 | >5.23 | >99.999 | >99.999 |
| Pseudomonas aeruginosa | 27853 | >5.17 | >5.17 | >99.999 | >99.999 |
| Serratia marcescens | 14756 | >5.25 | >5.25 | >99.999 | >99.999 |
| Proteus mirabilis | 25933 | >5.07 | >5.07 | >99.999 | >99.999 |
| Staphylococcus aureus | 6538 | >5.14 | >5.14 | >99.999 | >99.999 |
| Staphylococcus epidermidis | 12228 | >4.95 | >4.95 | >99.99 | >99.99 |
| Staphylococcus hominis | 27844 | >5.17 | >5.17 | >99.999 | >99.999 |
| Staphylococcus haemolyticus | 29970 | >5.07 | >5.07 | >99.999 | >99.999 |
| Micrococcus luteus | 7468 | >5.04 | >5.04 | >99.999 | >99.999 |
| Streptococcus pyogenes | 14289 | >4.20 | >4.20 | >99.99 | >99.99 |
| Enterococcus faecalis | 29212 | >5.17 | >5.17 | >99.999 | >99.999 |
| Enterococcus faecium | 35667 | >5.11 | >5.11 | >99.999 | >99.999 |
| Streptococcus pneumoniae | 6303 | >4.11 | >4.11 | >99.99 | >99.99 |
| Candida albicans | 10231 | >5.07 | >5.07 | >99.999 | >99.999 |

Reagents & Supplies

Dilution Fluid—D/E Neutralizing Broth or other appropriate neutralizing broth as required; Neutralizing Broth—D/E Neutralizing Broth or other appropriate neutralizing broth as required; Plating Medium—Aerobes: Tryptic Soy Agar (TSA), Sabouraud Dextrose Agar (SDA) Reinforced Clostridial Agar (RCA), Brain Heart Infusion Agar (BHIA), Blood agar, Schaedler Blood Agar with Vitamin K and Hemin or other agar as appropriate for the test organisms; Sterile Phosphate Buffered Saline (PBS) or sterile saline; Sterile bacteriological pipettes; Sterile Petri dishes; Sterile Test tubes; Test tube racks; Various other laboratory supplies including glassware, forceps, and pipettes; BD GasPak EZ Anaerobic Pouch System; BD GasPak Anaerobic Indicators

Equipment

Incubator (36-38° C.); Calibrated Timer displaying seconds; Colony Counter; Sterilizer; Vortex Mixer; BD GasPak EZ Large Containers.

Test Procedure

Neutralization Effectiveness

Inoculum Preparation for Neutralizer Effectiveness Studies Inoculum

Aerobic Organisms

The test organism were transferred twice (once every 18-24 hours) on Tryptic Soy Agar, Sabouraud Dextrose Agar or other appropriate agar as indicated herein, and incubated at approximately 36-38° C. for 24 hours minimum. The second transfer is made onto a TSA or SDA plate or slant and the inoculum prepared by washing the plate or slant with 5-10 mL of sterile PBS.

Anaerobic Organisms

The test organism were transferred twice (once every 2-3 days) on the appropriate anaerobic media as required by the organism and incubated at approximately 36-38° C. under anaerobic conditions in a BD GasPak Large Container with a BD GasPak EZ Anaerobic Pouch System and a BD GasPak Anaerobic Indicators. The second transfer is made onto the same media that was previously used for the organism and the inoculum prepared by washing the plate with 5-10 mL of sterile PBS or sterile saline.

The concentration of the test organism (aerobic or anaerobic) was adjusted spectrophotometrically in PBS or saline to a concentration of approximately 1×108 CFU/mL. The adjusted organism (10⁸) was serially diluted to approximately 103-104 CFU/mL; this is the inoculum that was utilized in the neutralizer effectiveness study.

Neutralizer Toxicity Test

Add 1 mL sterile saline to 9 mL neutralizer broth. Add the appropriate volume of the test organism to achieve <100 organisms and allow to stand for approximately 15 minutes. After 15 minutes, a 1.0 mL aliquot for aerobes or 0.1 mL aliquot for anaerobes was plated in duplicate using the media determined for each test organism. Plates were incubated at 36-38° C. for 24 hours minimum for aerobic organisms and for 2-3 days minimum under anaerobic conditions in a BD GasPak Large Container with a BD GasPak EZ Anaerobic Pouch System and a BD GasPak Anaerobic Indicators.

The neutralizer will be considered non-toxic up to and including the concentration at which organism recovery is within +/−50% of the sterile saline Viability Control Counts.

Neutralizer Effectiveness Test

The presence of active preservatives carried over from the challenged test article into the plating diluent and recovery medium during sampling may inhibit viable microorganisms and result in false-negative readings. Therefore, neutralizing agents should be incorporated into the plating diluent and/or recovery medium to inactivate preservatives and permit accurate enumeration of the microbial content. The choice of neutralizer will be based on the type of preservative system (e.g. lecithin for parabens or thiosulfate for halogens). The neutralizer should be evaluated prior to or concurrently with testing to determine if the preservative system is effectively neutralized. This can be done as follows:

Inoculate 1 gram of the test material into 9 mL of the appropriate neutralizing broth for each individual test organism to achieve a 1:10 and 1:100 dilution. Use sterile saline TS as a Viability Control without test material for each individual organism. Mix well.

Inoculate both the neutralized test material for each dilution (1:10 and 1:100) and the sterile saline Viability Control with test organisms to achieve a final concentration of test organisms in the range of <100 organisms. Testing shall be performed in duplicate.

Plate the appropriate dilutions in duplicate and add 20-25 mL of TSA or SDA agar tempered to 45° C. for plating unless specified to use a different agar medium. Allow to solidify and then incubate at 36-38° C. and microbial recovery incubation time for each test organism.

The spread plate technique shall be used for anaerobes or other fastidious organism using the media determined for that organism and incubated at the same incubation conditions. Plates for anaerobic organisms will be incubated 36-38° C. a minimum of 2-3 days under anaerobic conditions in a BD GasPak Large Container with a BD GasPak EZ Anaerobic Pouch System and a BD GasPak Anaerobic Indicators. A viability test blank for this control is run concurrently (see next section).

At the end of each incubation period, count the plates, take the average of the two plates. Express and record all findings as CFU/mL or g.

Calculate the % recovery of the neutralized test material versus the control. A suitable recovery is one that provides at least 50% of the sterile saline Viability Control. If there is at least 50% recovery for neutralized test material compared to the sterile saline Viability Control, then the neutralizer is considered effective. If there is less than 50%, a different neutralizing system should be evaluated and the test repeated.

Time Kill Assay

Test Sample Preparation

The test sample is tested neat. The testing is conducted at room temperature (20-25° C.) or as specified, and will be performed with 3 replicates with each replicate plated in duplicate. Testing is performed on 25 mL or gram aliquots of the test sample. Three containers of each test sample are tested for each test organism.

Inoculum Preparation for the Time Kill Tests

The test organisms are prepared as described herein for aerobic and anaerobic organisms. The concentration of the test organism is adjusted spectrophotometrically in PBS or saline to a concentration of approximately 1×108 CFU/ml.

Inoculum Enumeration

Plate the 10-6 and 10-7 aliquots in triplicate using the appropriate agar by either pour plate or spread plate. Plates are incubated at 36-38° C. for 24 hours minimum for aerobic organisms and for 2-3 days minimum under anaerobic conditions in a BD GasPak Large Container with a BD GasPak EZ Anaerobic Pouch System and a BD GasPak Anaerobic Indicators. Count colonies and record as CFU/mL.

If the test period runs longer than 60 minutes repeat the enumeration series of plates and identify each series of plates as either “pre-test” or “post-test” count. The beginning and end counts must be within one log 10 for test to be valid.

Time Kill Procedure

Inoculate 25 grams or mL of the test sample with 0.250 mL of test organism suspension (resulting in approximately 106 CFU/mL). If sufficient test material is not available a lower quantity can be used such as 10 or 15 grams. The inoculum volume is reduced to achieve the same concentration based on the amount of test material used.

At 15 and 30 seconds, transfer 1.0 mL of sample/bacteria mixture from beaker into a 9.0 mL D/E neutralizing broth or other appropriate neutralizing broth.

Serial dilute in D/E neutralizing broth or other appropriate neutralizing broth.

Plate the 10-1, 10-2, 10-3 and 10-4 dilutions into duplicate Petri plates using either pour plate or spread plate technique using the appropriate agar for the test organism.

Plates are incubated at 36-38° C. for 24 hours minimum for aerobic organisms and for 2-3 days minimum under anaerobic conditions in a BD GasPak Large Container with a BD GasPak EZ Anaerobic Pouch System and a BD GasPak Anaerobic Indicators

Repeat steps 1 through 7 on 2 additional aliquots of test material for a total of 2 replicates.

Blank: 25 gram or ml aliquots of sterile DI water are processed as described herein as a control. If a different volume of test material is used, the volume of sterile DI water and inoculum volume will also be adjusted.

Calculations/Data Analysis

Results are reported as the number of surviving organisms over time. Plates containing 30-300 colonies per plate are used for calculations where possible. The number of surviving organisms at a time will be determined by averaging the plate counts, correcting for dilution, and log transforming this corrected value. This log transformed value are expressed as the result. Reduction in counts after exposure to test material compared to the blank indicate efficacy of the test material.

All patents, patent applications, and published references cited herein are hereby incorporated by reference in their entirety. While the methods of the present disclosure have been described in connection with the specific embodiments thereof, it will be understood that it is capable of further modification. Further, this application is intended to cover any variations, uses, or adaptations of the methods of the present disclosure, including such departures from the present disclosure as come within known or customary practice in the art to which the methods of the present disclosure pertain.

### REFERENCES

1. J. Chin, B. C. Jiang, I. Mufidah, S. F. Persada, and B. A. Noer, The investigation of consumers' behavior intention in using green skincare products: A pro-environmental behavior model approach. Sustain. 10 (11) (2018).
2. M. C. Cervellon, and L. Carey, Consumers' perceptions of ‘green’: Why and how consumers use eco-fashion and green beauty products. Crit. Stud. Fash. Beauty. 2, 117-138 (2011).
3. J. D. Desai, and I. M. Banat, Microbial production of surfactants and their commercial potential. Microbiol. Mol. Biol. Rev. 61 (1), 47-64 (1997).
4. A. Varvaresou and K. Iakovou, Biosurfactants in cosmetics and biopharmaceuticals. Lett.

Appl. Microbiol. 61 (3), 214-223 (2015).
5. M. Pacwa-Plociniczak, G. A. Plaza, Z. Piotrowska-Seget, and S. S. Cameotra, Environmental Applications of Biosurfactants: Recent Advances. Int. J. Mol. Sci. 12(1), 633-654 (2011).
6. D. Santos, R. Rufino, J. Luna, V. Santos, and L. Sarubbo, Biosurfactants: Multifunctional Biomolecules of the 21st Century. Int. J. Mol. Sci. 17(3), 401 (2016).
7. S. Gatard, M. N. Nasir, M. Deleu et al. Bolaamphiphiles Derived from Alkenyl L-Rhamnosides and Alkenyl D-Xylosides: Importance of the Hydrophilic Head. Molecules. 18(5), 6101-6112 (2013).
8. S. Matsumura, K. Imai, S. Yoshikawa, K. Kawada, and T. Uchibor, Surface activities, biodegradability and antimicrobial properties of n-alkyl glucosides, mannosides and galactosides. J. Am. Oil Chem. Soc. 67(12), 996-1001 (1990).
9. M. A. Grolitzer, Glucoside surfactants. (1985).
10. M. Chvapli, and Z. Eckmayer, Role of proteins in cosmetics. Int. J. Cosmet. Sci. 7(2), 41-49 (1985).
11. S. Inoue, K. Tanaka, F. Arisaka et al. Silk fibroin of Bombyx mori is secreted, assembling a high molecular mass elementary unit consisting of H-chain, L-chain, and P25, with a 6:6:1 molar ratio. J. Biol. Chem. 275(51), 40517-40528 (2000).
12. Y. Suzuki, T. Yamazaki, A. Aoki, H. Shindo, and T. Asakura, NMR study of the structures of repeated sequences, GAGXGA (X═S, Y, V), in Bombyx mori liquid silk. Biomacromolecules. 15(1), 104-112 (2014).
13. J. Wen, J. Yao, X. Chen, and Z. Shao, Silk Fibroin Acts as a Self-Emulsifier to Prepare Hierarchically Porous Silk Fibroin Scaffolds through Emulsion—Ice Dual Templates. ACS Omega. 3(3), 3396-3405 (2018).
14. L. Wang, X. Hongen, X. Qiao et al. Interfacial rheology of natural silk fibroin at air/water and oil/water interfaces. Langmuir. 28(1), 459-467 (2012).
15. X. Qiao, L. Wang, Z. Shao, K. Sun, and R. Miller, Stability and rheological behaviors of different oil/water emulsions stabilized by natural silk fibroin. Colloids Surfaces A Physicochem. Eng. Asp. 475, 84-93 (2015).
16. F. P. Seib, Silk nanoparticles—an emerging anticancer nanomedicine. AIMS Bioeng. 42(2), 239-258 (2017).
17. H. J. Jin, and D. L. Kaplan, Mechanism of silk processing in insects and spiders. Nature. 424(6952), 1057-1061 (2003).
18. I. Greying, C. Dicko, A. Terry, P. Callow, and F. Vollrath, Small angle neutron scattering of native and reconstituted silk fibroin. Soft Matter. 6(18), 4389-4395 (2010).
19. J. Alamed, D. J. McClements, and E. A. Decker, Influence of heat processing and calcium ions on the ability of EDTA to inhibit lipid oxidation in oil-in-water emulsions containing omega-3 fatty acids. Food Chem. 95(4), 585-590 (2006).
20. Y. S. Gu, L. Regnier, and D. J. McClements, Influence of environmental stresses on stability of oil-in-water emulsions containing droplets stabilized by β-lactoglobulin-ι-carrageenan membranes. J. Colloid Interface Sci. 286(2), 551-558 (2005).
21. J. J. Rao, Z. M. Chen, and B. C. Chen, Modulation and stabilization of silk fibroin-coated oil-in-water emulsions. Food Technol. Biotechnol. 47(4), 413-420 (2009).
22. H. J. Jin, and D. L Kaplan, Mechanism of silk processing in insects and spiders. Nature.

424(6952), 1057-1061 (2003).
23. F. Vollrath, and D. P. Knight, Liquid crystalline spinning of spider silk. Nature 0.410(6828), 541-548 (2001).
24. R. E. Black, F. J. Hurley, and D. C. Havery, Occurrence of 1,4-Dioxane in Cosmetic Raw Materials and Finished Cosmetic Products. J. AOAC Int. 84(6), 666-670 (2001).
25. P. A. Cornwell, A review of shampoo surfactant technology: consumer benefits, raw materials and recent developments. Int. J. Cosmet. Sci. 40(1), 16-30 (2018).
26. D. A. Adejokun, and K. Dodou, Quantitative Sensory Interpretation of Rheological

Parameters of a Cream Formulation. Cosmetics. 7(1), 2 (2020).
27. G. Evmenenko, E. Theunissen, K. Mortensen, and H. Reynaers, SANS study of surfactant ordering in κ-carrageenan/cetylpyridinium chloride complexes. Polymer. 42(7), 2907-2913 (2001).
28. F. Begum, and S. Amin, Investigating the influence of polysorbate 20/80 and polaxomer P188 on the surface & interfacial properties of bovine serum albumin and lysozyme. Pharm. Res. 36(7), 107 (2019).
29. B. B. White, and J. C. T. Kwak, Swelling of hydrophobically modified poly(acrylamide) and poly(acrylamide)-co-(acrylic acid) gels in surfactant solutions. Colloid Polym. Sci. 277(8), 785-791 (1999).

1. A silk personal care composition comprising silk fibroin fragments having an average weight average molecular weight selected from between about 1 kDa to about 5 kDa, from between about 5 kDa to about 10 kDa, from between about 6 kDa to about 17 kDa, from between about 10 kDa to about 15 kDa, from between about 14 kDa to about 30 kDa, from between about 15 kDa to about 20 kDa, from between about 17 kDa to about 39 kDa, from between about 20 kDa to about 25 kDa, from between about 25 kDa to about 30 kDa, from between about 30 kDa to about 35 kDa, from between about 35 kDa to about 40 kDa, from between about 39 kDa to about 54 kDa, from between about 39 kDa to about 80 kDa, from between about 40 kDa to about 45 kDa, from between about 45 kDa to about 50 kDa, from between about 50 kDa to about 55 kDa, from between about 55 kDa to about 60 kDa, from between about 60 kDa to about 100 kDa, and from between about 80 kDa to about 144 kDa, and a polydispersity ranging from 1 to about 5;
from 0 to 500 ppm lithium bromide;
from 0 to 500 ppm sodium carbonate; and
a carrier.

2. The silk personal care composition of claim 1, wherein the silk fibroin fragments have a polydispersity ranging from 1.0 to about 1.5, from about 1.5 to about 2.0, from about 1.5 to about 3.0, from about 2.0 to about 2.5, or from about 2.5 to about 3.0.

3. The silk personal care composition of claim 1 or claim 2, wherein the silk fibroin fragments are present in the composition in an amount ranging from about 0.001 wt. % to about 10.0 wt. % by the total weight of the silk personal care composition.

4. The silk personal care composition of any one of claims 1 to 3, wherein the silk fibroin fragments are present in the composition in an amount ranging from about 0.001 wt. % to about 0.01 wt. %, from about 0.01 wt. % to about 0.1 wt. %, from about 0.1 wt. % to about 1.0 wt. %, from about 1.0 wt. % to about 2.0 wt. %, from about 2.0 wt. % to about 3.0 wt. %, from about 3.0 wt. % to about 4.0 wt. %, from about 4.0 wt. % to about 5.0 wt. %, or from about 5.0 wt. % to about 6.0 wt. % by the total weight of the silk personal care composition.

5. The silk personal care composition of any one of claims 1 to 3, wherein the silk fibroin fragments are present in the composition in an amount of about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, or about 0.1 wt. %.

6. The silk personal care composition of any one of claims 1 to 5, wherein the silk personal care composition further comprises about 0.001% (w/w) to about 10% (w/w) sericin by the total weight of the silk personal care composition.

7. The silk personal care composition of any one of claims 1 to 5, wherein the silk personal care composition further comprises about 0.001% (w/w) to about 10% (w/w) sericin by the total weight of the silk fibroin fragments.

8. The silk personal care composition of any one of claims 1 to 7, wherein the silk fibroin fragments in the silk personal care composition do not spontaneously or gradually gelate and do not visibly change in color or turbidity when in an aqueous solution for at least 10 days prior to being formulated into the silk personal care composition.

9. The silk personal care composition of any one of claims 1 to 8, wherein the composition comprises an oil phase.

10. The silk personal care composition of any one of claims 1 to 9, wherein the composition comprises an aqueous phase.

11. The silk personal care composition of any one of claims 1 to 9, wherein the silk personal care composition comprises an “oil-in-water” type emulsion or a “water-in-oil” type emulsion.

12. The silk personal care composition of any one of claims 1 to 9, wherein the silk personal care composition comprises a gel phase.

13. The silk personal care composition of any one of claims 1 to 12, wherein the silk personal care composition further comprises an emulsifier, a surfactant, or both.

14. The silk fibroin fragment composition of claim 13, wherein the surfactant is selected from the group consisting of C16-C24 fatty alcohol, soy lecithin, egg lecithin, sucrose ester, cetearyl glucoside, caprylyl/capryl glucoside, decyl glucoside, sucrose laurate, sucrose palmitate, sucrose stearate, sucrose cocoate, sorbitan monostearate, cocobetaine, and combinations thereof.

15. The silk personal care composition of any one of claims 1 to 14, further comprising an additive selected from butanediol, propanediol, ethanediol, glycerol, butantetraol, xylitol, D-sorbitol, inositol, polyethylene glycol, hydroxyethyl cellulose, hydroxypropyl methylcellulose, dextran, gelatin, carboxymethyl cellulose, propylene glycol, polysorbate 80, polyvinyl alcohol, povidone, saponin, sucrose, fructose, maltose, carrageenan, chitosan, alginate, hyaluronic acid, and combinations thereof.

16. The silk personal care composition of any one of claims 1 to 15, further comprising one or more solvents selected from methanol, ethanol, propanol, isopropanol, acetonitrile, and combinations thereof.

18. The silk personal care composition of any one of claims 1 to 16, further comprising a gelling and/or thickening agent.

19. The silk personal care composition of claim 18, wherein the gelling and/or thickening agent comprises a hydroxy alkyl cellulose.

20. The silk personal care composition of claim 18, wherein the gelling and/or thickening agent comprises one or more of hydroxy methyl cellulose, hydroxyethyl cellulose, hydroxypropyl cellulose, hydroxypropyl methylcellulose, dextran, gelatin, carboxymethyl cellulose, propylene glycol, polysorbate 80, polyvinyl alcohol, povidone, sucrose, fructose, maltose, carrageenan, chitosan, alginate, hyaluronic acid, gum arabic, galactomannans, xanthan gum, pectin, and combinations thereof.

21. The silk personal care composition of any one of claims 1 to 20, wherein the silk personal care composition further comprises an emulsifiable component.

22. The silk personal care composition of claim 21, wherein the emulsifiable component comprises a hydrophobic emulsifiable component, a hydrophilic emulsifiable component, an amphiphilic emulsifiable component, or a combination thereof.

23. The silk personal care composition of claim 21, wherein the emulsifiable component comprises one or more of an oil, a fat, a wax, a lipid, and combinations thereof.

24. The silk personal care composition of claim 23, wherein the oil is selected from hydrocarbon oil, mineral oil, silicon oil, fatty acid having 8 to 32 carbon atoms, fatty alcohol having 8 to 32 carbon atoms, synthetic ester oil derived from the esterification product of fatty acid having 8 to 32 carbon atoms and an alcohol, fatty acid glyceride, glyceryl trioctanoate, glyceryl triisopalmitate, cholesteryl isostearate, isopropyl palmitate, isopropyl myristate, neopentyl glycol dicaprate, isopropyl isostearate, octadecyl myristate, cetyl 2-ethylhexanoate, cetearyl isononanoate, cetearyl octanoate, isononyl isononanoate, isotridecyl isononanoate, glyceryl tri-2-ethylhexanoate, glyceryl tri(caprylatelcaprate), diethylene glycol monoethyl ether oleate, dicaprylyl ether, caprylic acid/capric acid propylene glycol diester, isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexylate, dipentaerythritol fatty acid ester, N-alkyl glycol monoisostearate, neopentyl glycol dicaprate, diisostearyl malate, glyceryl di-2-heptylundecanoate, trimethylolpropane tri-2-ethylhexylate, trimethylolpropane triisostearate, pentaneerythritol tetra-2-ethylhexylate, glyceryl tri-2-ethylhexylate, trimethylolpropane triisostearate, cetyl 2-ethylhexanoate, 2-ethylhexyl palmitate, glyceryl trimyristate, tri-2-heptylundecanoic glyceride, oleyl oleate, cetostearyl alcohol, 2-heptylundecyl palmitate, diisopropyl adipate, N-lauroyl-L-glutamic acid-2-octyldodecyl ester, di-2-heptylundecyl adipate, ethyl laurate, di-2-ethylhexyl cebatate. 2-hexyldecyl myristate, 2-hexyldecyl palmitate, 2-hexyldecyl adipate, diisopropyl cebatate, 2-ethylhexyl succinate, ethyl acetate, butyl acetate, amyl acetate and triethyl |citrate.

25. The silk personal care composition of claim 23, wherein the fat is selected from liquid fat, solid fat, avocado oil, tsubaki oil, turtle oil, macadamia nut oil, corn oil, mink oil, olive oil, rape seed oil, egg yolk oil, sesame seed oil, persic oil, wheat germ oil, sasanqua oil, castor oil, linseed oil, safflower oil, cotton seed oil, perilla oil, soybean oil, peanut oil, tea seed oil, kaya oil, rice bran oil, Chinese wood oil, Japanese wood oil, jojoba oil, germ oil, sweet almond oil, rosehip seed oil, calendula oil, grape seed oil, apricot kernel oil, flaxseed oil, hazelnut oil, walnut oil, pecan nut oil, sesame oil, emu oil, coconut oil, sunflower oil, canola oil, algae oil, cacao butter, horse tallow, hardened coconut oil, palm oil, beef tallow, sheep tallow, pork tallow, hardened beef tallow, palm kernel oil, Japanese core wax, hydrogenated castor oil, and combinations thereof.

26. The silk personal care composition of claim 23, wherein the wax is selected from butter, petrolatum, polyethylene wax, polypropylene wax, Japanese wax, beeswax, candelilla wax, paraffin wax, ozokerite, microcrystalline wax, carnauba wax, cotton wax, esparto wax, bayberry wax, tree wax, whale wax, montan wax, bran wax, lanolin wax, kapok wax, lanolin acetate, sugar cane wax, lanolin fatty acid isopropyl ester, hexyl laurate, reduced lanolin, jojoba wax, hard lanolin, shellac wax, POE lanolin alcohol ether, lanolin alcohols with 40 moles ethylene oxide, lanolin alcohols with 65-70 moles ethylene oxide, POE lanolin alcohol acetate, POE cholesterol ether, lanolin fatty acid, POE hydrogenated lanolin alcohol ether, and combinations thereof.

27. The silk personal care composition of claim 23, wherein the lipid is selected from phospholipid, polymer-lipid conjugate, carbohydrate-lipid conjugate, dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-dimyristoyl-sn-glycero-3-phosphorylglycerol (DMPG); 1,2-distearoyl-sn-glycero-3-phosphoethanolamine (DSPE); 1,2-dioleoyl-sn-glycero-3-phosphocholine (DOPC); 1,2-dioleoyl-sn-glycero-3-phosphoethanolamine (DOPE); 1,2-dipalmitoyl-sn-glycero-3-phospho-(1′-rac-glycerol) (DPPG); 1,2-distearoyl-sn-glycero-3-phosphocholine (DSPC), distearoylphosphoethanolamine conjugated with polyethylene glycol (DSPE-PEG); phosphatidylserine (PS), phosphatidylethanolamine (PE), phosphatidylglycerol (PG), phosphatidylcholine (PC), cholesterol, 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-diarachidyl-sn-glycero-3-phosphoethanolamine (20:0 PE), 1-stearoyl-2-linoleoyl-sn-glycero-3-phosphoethanolamine, 1,2-diheptadecanoyl-sn-glycero-3-phosphocholine (17:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

28. The silk personal care composition of claim 23, wherein the lipid is a phospholipid selected from soy lecithin and egg lecithin.

29. The silk personal care composition of any one of claims 1 to 28, further comprising a density matching agent or a weighting agent selected from ester gum (EG), damar gum (DG), sucrose acetate isobutyrate (SAIB), brominated vegetable oil (BVO), and combinations thereof.

30. The silk personal care composition of claim 29, wherein the weighting agent concentrations required to match the oil and aqueous phase densities is of about 10.0 wt. % to about 25.0 wt. % for BVO, about 35.0 wt. % to about 55.0 wt. % for EG, about 35.0 wt. % to about 55.0 wt. % for DG, and about 25.0 wt. % to about 45.0 wt. % for SAIB.

31. The silk personal care composition of any one of claims 1 to 30, wherein a portion of the silk fibroin fragments has a hydrophilic-lipophilic balance (HLB) value selected from the group consisting of from 0 to about 3, from about 3 to about 6, from about 6 to about 9, from about 9 to about 12, from about 12 to about 15, from about 15 to about 18, and greater than 18.

32. The silk personal care composition of any one of claims 1 to 30, wherein a portion of the silk fibroin fragments has a HLB value selected from the group consisting of 0, about 1, about 2, about 3, about 4, about 5, about 6, about 7, about 8, about 9, about 10, about 11, about 12, about 13, about 14, about 15, about 16, about 17, about 18, about 19, and about 20.

33. The silk personal care composition of any one of claims 1 to 30, wherein a portion of the silk fibroin fragments has a HLB value ranging from about 8 to about 18.

34. The silk personal care composition of any one of claims 1 to 30, wherein a portion of the silk fibroin fragments has a HLB value ranging from 0 to about 8.

35. The silk personal care composition of any one of claims 1 to 34, wherein a fraction of silk fibroin fragments is substantially solid.

36. The silk personal care composition of claim 35, wherein the substantially solid silk fibroin fragments are formulated into particles.

37. The silk personal care composition of any one of claims 1 to 36, wherein the silk personal care composition is formulated as an oral care composition, a skin care composition, a sanitizing composition, a hair care composition, a cosmetic composition, a makeup composition, a sun care composition, a deodorant, an antiperspirant composition, a nail cosmetic composition, a skin cleansing composition, an aromatic cosmetic, or a bath cosmetic composition.

38. The silk personal care composition of any one of claims 1 to 35, wherein the silk personal care composition is formulated into personal care product selected from a beauty soap, a soap bar, a soap solution, a soap gel, a facial wash, a hand wash, a body wash, a hand sanitizer, a cleansing wipe, a feminine hygiene product, a cleansing pad, a cleansing foam, a rinse, a cleansing lotion, a cleansing milk, a cleansing gel, a cleansing soap bar, an exfoliating product, a bath and shower soap in bar, a cream, an emulsion, a shaving or after-shave cream, a foam, a conditioner, a cologne, a shaving or after-shave lotion, a perfume, a cosmetic oil, a facial mask, a moisturizer, an anti-wrinkle, an eye treatment, a tanning cream, a tanning lotion, a tanning emulsion, a sunscreen cream, a sunscreen lotion, a sunscreen emulsion, a tanning oil, a sunscreen oil, a hand lotion, a body lotion, a color cosmetic, a mascara, a lipstick, a lip liner, an eye shadow, an eye-liner, a rouge, a face powder, a foundation, a blush, perfume, bath soap in bar, bath product, a toothpaste, a dentifrice, a tooth powder, an oral gel, an aqueous gel, a non-aqueous gel, a mouth rinse, a mouth spray, a plaque removing liquid, a denture product, a dental solution, a lozenge, oral tablet, a chewing gum, a candy, a fast-dissolving film, a strip, a dental floss, a tooth glossing product, a finishing product, an impregnated dental implement, a remineralizing gel, a remineralizing mouthwash, a remineralizing tooth powder, a remineralizing chewing gum, a remineralizing lozenge, a remineralizing toothpaste, a antiperspirant stick, a roll-on deodorant, a powder deodorant, a gel deodorant, an aerosol deodorant, a paste deodorant, and a cream nail polish, and a nail polish remover.

39. The silk personal care composition of claim 38, wherein the silk personal care product contains at most 13 different ingredients in total.

40. The silk personal care composition of claim 38, wherein the silk personal care product contains less than twelve different ingredients in total.

41. The silk personal care composition of claim 37, wherein the oral care composition further comprises an additive selected from a filler, a diluent, a remineralizing agent, an anti-calculus agent, an anti-plaque agent, a buffer, an abrasive, an alkali metal bicarbonate salt, a binder, a thickening agent, a humectant, a whitening agent, a bleaching agent, a stain removing agent, a surfactant, titanium dioxide, a flavoring agent, xylitol, a coloring agent, a foaming agent, a sweetener, an antibacterial agent, a preservative, a vitamin, a pH-adjusting agent, an anti-caries agent, a desensitizing agent, a coolant, a salivating agent, a warming agent, a numbing agent, a chelating agent, and combinations thereof.

42. The silk personal care composition of claim 37, wherein the oral care composition is formulated as a product selected from a toothpaste, a dentifrice, a tooth powder, an oral gel, an aqueous gel, a non-aqueous gel, a mouth rinse, a mouth spray, a plaque removing liquid, a denture product, a dental solution, a lozenge, an oral tablet, a chewing gum, a candy, a fast-dissolving film, a strip, a dental floss, a tooth glossing product, a finishing product, and an impregnated dental implement.

43. The silk personal care composition of claim 37, wherein the oral care composition is formulated as a toothpaste comprising a tooth care active agent selected from an abrasive, an anti-calculus agent, an anti-plaque agent, a humectant, a whitening agent, an anti-caries agent, a desensitizing agent, a coolant, a salivating agent, a warming agent, a numbing agent, and combinations thereof.

44. The silk personal care composition of claim 37, wherein the oral care composition is formulated as a tooth remineralization product comprising a therapeutically effective amount of a remineralizing agent.

45. The silk personal care composition of claim 44, wherein the remineralizing agent is selected from the group consisting of fluoride, calcium source compound, phosphate source compound, calcium carbonate, sodium hydrogen phosphate, sodium dihydrogen phosphate, potassium hydrogen phosphate, potassium dihydrogen phosphate, amorphous calcium phosphate (ACP), tricalcium phosphate, casein phosphoprotein-ACP, bioactive glass, calcium sodium phosphosilicate, arginine bicarbonate-calcium carbonate complex, and combinations thereof.

46. A silk oral care article comprising the silk personal care composition of any one of claims 42 to 45 and a support.

47. The silk oral care article of claim 46, wherein the support comprises a pellet, wood, metal, plastic, paper, yarn, thread, fiber, a fabric layer, a film, and/or a hydrogel.

48. The silk oral care article of claim 47, wherein the fabric layer comprises one or more of a natural fiber or yarn comprising one or more of cotton and wool, or a synthetic fiber or yarn comprising one or more of polyester, nylon, polyester-polyurethane copolymer, polyamide, polyaramid, polytetrafluoroethylene, polyethylene, polypropylene, polyurethane, silicone, polyurethane, polyethyleneglycol, polypropylene (PP), thermoplastic polyurethane (TPU), polyethylene (PE), Nylon and combinations thereof.

49. The silk personal care composition of claim 37, wherein the silk personal care composition is formulated as a skin cleansing composition.

50. The silk personal care composition of claim 49, wherein the skin cleansing composition further comprises an additive selected from a cleansing surfactant, a soap base, a detergent, a lathering surfactant, a skin conditioning agent, an oil control agent, an anti-acne agent, an astringent, an exfoliating particle or agent, a skin calming agent, a plant extract, an essential oil, a coolant, a humectant, a moisturizer, a structurant, a gelling agent, an antioxidant, an anti-aging compound, a skin lightening agent, a preservative, a filler, a fragrance, a thickener, a coloring agent, an antimicrobial agent, and combinations thereof.

51. The silk personal care composition of claim 49, wherein the skin cleansing composition is formulated as a product selected from a hand sanitizer, a hand wash, a wash gel, a cleansing lotion, a cleansing milk, a cleansing gel, a cleansing soap bar, an exfoliating product, a bath and shower soap in bar, a body wash, a hand wash, a cleansing wipe, a cleansing pad, and a bath product.
